(19) United States
(12) Patent Application Publication
(10) Pub. No.: US 2019/0389836 A1
(43) Pub. Date: Dec. 26, 2019

(54) NRF2 ACTIVATOR

(71) Applicant: BIOGEN MA INC., Cambridge, MA (US)

(72) Inventors: Andrew George Capacci, Cambridge, MA (US); Michael Dechantsreiter, Allston, MA (US); Istvan Enyedy, Milton, MA (US); John H. Jones, Framingham, MA (US); Edward Yin-Shiang Lin, Ashland, MA (US); Brian Stuart Lucas, Arlington, MA (US); Bin Ma, Arlington, MA (US)

(21) Appl. No.: 16/481,772

(22) PCT Filed: Jan. 29, 2018
(86) PCT No.: PCT/US2018/015738

§ 371 Date: Jul. 29, 2019

Related U.S. Application Data
(60) Provisional application No. 62/452,120, filed on Jan. 30, 2017.

(51) Int. Cl. C07D 401/06; C07D 401/14; C07D 413/14; C07D 417/14; C07D 215/14; C07D 405/14; C07D 471/04; C07D 451/02

(52) U.S. Cl. CPC C07D 401/06; C07D 401/14; C07D 413/14; C07D 451/02; C07D 215/14; C07D 405/14; C07D 471/04; C07D 417/14

(57) ABSTRACT

Provided are tetrahydroisoquinoline derivatives as Nrf2 activators.

### RELATED APPLICATION INFORMATION

This application claims priority to U.S. Provisional Application No. 62/452,120, filed Jan. 30, 2017. The contents of this application are incorporated herein by reference.

Nuclear factor erythroid 2 (NF-E2)-related factor 2 (Nrf2) belongs to the Cap ‘N’ Collar (CNC) family of transcription factors and contains a conserved basic leucine zipper (bZIP) structure. The main function of Nrf2 is to activate the cellular antioxidant response by inducing the production of proteins that are able to combat the harmful effects of oxidative stress.

Activation of the Nrf2 pathway to treat diseases caused by oxidative stress, such as a neurodegenerative disease, inflammation and/or an inflammatory disease, an autoimmune disease, an ischemic fibrotic disease, a cancer, premature aging, a cardiovascular disease, a liver disease, a hemoglobinopathy and a metabolic disorder, is being studied.

Moreover, Nrf2 activation has been shown to upregulate fetal hemoglobin which can ameliorates symptoms of hemoglobinopathy such as sickle cell disease and thalassemia (e.g. beta-thalassemia).

Therefore, a need exists for Nrf2 activators to treat these diseases.

### SUMMARY

Disclosed herein are potent activators of Nrf2 (see Example 163). These compounds can be used in the treatment of diseases treatable by activating Nrf2.

A first embodiment of the invention is a compound of Formula A:

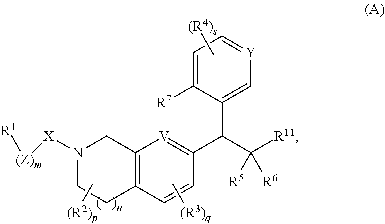

or a pharmaceutically acceptable salt thereof, wherein
V is CH or N;
R¹ is a 3 to 12-membered carbocyclyl, a 3 to 12-membered heterocyclyl, or —N(R^{1a})₂, wherein the 3 to 12-membered carbocyclyl and 3 to 12-membered heterocyclyl are each optionally substituted with one or more R¹⁵;
X is —C(O)— or —S(O)₂—;
R² is halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{2a}, —C(S)R^{2a}, —C(O)OR^{2a}, —C(S)SR^{2a}, —C(O)SR^{2a}, —C(S)OR^{2a}, —SC(O)R^{2a}, —OC(S)R^{2a}, —SC(S)R^{2a}, —C(O)N(R^{2a})₂, —OR^{2a}, —SR^{2a}, —N(R^{2a})₂, —N(R^{2a})OR^{2a}, —N(R^{2a})S(O)₂R^{2a}, —N(R^{2a})C(O)R^{2a}, —N(R^{2a})N(R^{2a})₂, —N(R^{2a})C(O)OR^{2a}, —N(R^{2a})C(O)N(R^{2a})₂, —S(O)₂R^{2a}, —S(O)R^{2a}, —S(O)N(R^{2a})₂, —S(O)₂N(R^{2a})₂, —N⁺(R^{2a})₃, —S⁺(R^{2a})₂, or —Si(R^{2a})₃; or two R² groups, attached to non-adjacent ring carbon atoms and taken together with the two non-adjacent ring carbon atoms, form a non-saturated heterocyclic, bridged bicyclyl, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R²⁵, and wherein the non-saturated heterocyclic, bridged bicyclyl is optionally substituted with one or more R⁹;
R³ is halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{3a}, —C(S)R^{3a}, —C(O)OR^{3a}, —C(S)SR^{3a}, —C(O)SR^{3a}, —C(S)OR^{3a}, —SC(O)R^{3a}, —OC(S)R^{3a}, —SC(S)R^{3a}, —C(O)N(R^{3a})₂, —OR^{3a}, —SR^{3a}, —N(R^{3a})₂, —N(R^{3a})OR^{3a}, —N(R^{3a})S(O)₂R^{3a}, —N(R^{3a})C(O)R^{3a}, —N(R^{3a})N(R^{3a})₂, —N(R^{3a})C(O)OR^{3a}, —N(R^{3a})C(O)N(R^{3a})₂, —S(O)₂R^{3a}, —S(O)R^{3a}, —S(O)N(R^{3a})₂, —S(O)₂N(R^{3a})₂, —N⁺(R^{3a})₃, —S⁺(R^{3a})₂, or —Si(R^{3a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R³⁵;
R⁴ is halo, —NO₂, —CN, —N₃, C₁₋₁₂alkyl, C₂₋₁₂alkenyl, C₂₋₁₂alkynyl, —C(O)R^{4a}, —C(S)R^{4a}, —C(O)OR^{4a}, —C(S)SR^{4a}, —C(O)SR^{4a}, —C(S)OR^{4a}, —SC(O)R^{4a}, —OC(S)R^{4a}, —SC(S)R^{4a}, —C(O)N(R^{4a})₂, —OR^{4a}, —SR^{4a}, —N(R^{4a})₂, —N(R^{4a})OR^{4a}, —N(R^{4a})S(O)₂R^{4a}, —N(R^{4a})C(O)R^{4a}, —N(R^{4a})N(R^{4a})₂, —N(R^{4a})C(O)OR^{4a}, —N(R^{4a})C(O)N(R^{4a})₂, —S(O)₂R^{4a}, —S(O)R^{4a}, —S(O)N(R^{4a})₂, —S(O)₂N(R^{4a})₂, —N⁺(R^{4a})₃, —S⁺(R^{4a})₂, or —Si(R^{4a})₃; or two R⁴ groups, attached to adjacent ring carbon atoms and taken together with the two adjacent ring carbon atoms, form triazolyl, 2,5-dihydrofuranyl, 2,3-dihydro-1,4-dioxinyl, 3,4-dihydro-2,4-pyranyl, 1,2,3,6-tetrahydropyridinyl, 1H-imidazolyl or pyrazinyl, wherein the C₁₋₁₂alkyl, C₂₋₁₂alkenyl, and C₂₋₁₂alkynyl are each optionally substituted with one or more R⁴⁵, and wherein the triazolyl, 2,5-dihydrofuranyl, 2,3-dihydro-1,4-dioxinyl, 3,4-dihydro-2,4-pyranyl, 1,2,3,6-tetrahydropyridinyl, 1H-imidazolyl and pyrazinyl are each optionally substituted with one or more R⁹;
R⁵ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{5a}, —C(S)R^{5a}, —C(O)OR^{5a}, —C(S)SR^{5a}, —C(O)SR^{5a}, —C(S)OR^{5a}, —SC(O)R^{5a}, —OC(S)R^{5a}, —SC(S)R^{5a}, —C(O)N(R^{5a})₂, —OR^{5a}, —SR^{5a}, —N(R^{5a})₂, —N(R^{5a})OR^{5a}, —N(R^{5a})S(O)₂R^{5a}, —N(R^{5a})C(O)R^{5a}, —N(R^{5a})N(R^{5a})₂, —N(R^{5a})C(O)OR^{5a}, —N(R^{5a})C(O)N(R^{5a})₂, —S(O)₂R^{5a}, —S(O)R^{5a}, —S(O)N(R^{5a})₂, —S(O)₂N(R^{5a})₂, —N⁺(R^{5a})₃, —S⁺(R^{5a})₂, or —Si(R^{5a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁵⁵;
R⁶, in each occurrence, is independently H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{6a}, —C(S)R^{6a}, —C(O)OR^{6a}, —C(S)SR^{6a}, —C(O)SR^{6a}, —C(S)OR^{6a}, —SC(O)R^{6a}, —OC(S)R^{6a}, —SC(S)R^{6a}, —C(O)N(R^{6a})₂, —OR^{6a}, —SR^{6a}, —N(R^{6a})₂, —N(R^{6a})OR^{6a}, —N(R^{6a})S (O)₂R^{6a}, —N(R^{6a})C(O)R^{6a}, —N(R^{6a})N(R^{6a})₂, —N(R^{6a})C(O)OR^{6a}, —N(R^{6a})C(O)N(R^{6a})₂, —S(O)₂R^{6a}, —S(O)R^{6a}, —S(O)N(R^{6a})₂, —S(O)₂N(R^{6a})₂, —N⁺(R^{6a})₃, —S⁺(R^{6a})_{2,} or —Si(R^{6a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁶⁵;
R⁷ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{7a}, —C(S)R^{7a}, —C(O)OR^{7a}, —C(S)SR^{7a}, —C(O)SR^{7a}, —C(S)OR^{7a}, —SC(O)R^{7a}, —OC(S)R^{7a}, —SC(S)R^{7a}, —C(O)N(R^{7a})₂, —OR^{7a}, —SR^{7a}, —N(R^{7a})₂, —N(R^{7a})OR^{7a}, —N(R^{7a})S(O)₂R^{7a}, —N(R^{7a})C(O)R^{7a}, —N(R^{7a})N(R^{7a})₂, —N(R^{7a})C(O)OR^{7a}, —N(R^{7a})C(O)N(R^{7a})₂, —S(O)₂R^{7a}, —S(O)R^{7a}, —S(O)N(R^{7a})₂, —S(O)₂N(R^{7a})₂, —N⁺(R^{7a})₃, —S⁺(R^{7a})_{2,} or —Si(R^{7a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁷⁵;
Y is N or CR⁸, wherein R⁸ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{8a}, —C(S)R^{8a}, —C(O)OR^{8a}, —C(S)SR^{8a}, —C(O)SR^{8a}, —C(S)OR^{8a}, —SC(O)R^{8a}, —OC(S)R^{8a}, —SC(S)R^{8a}, —C(O)N(R^{8a})₂, —OR^{8a}, —SR^{8a}, —N(R^{8a})₂, —N(R^{8a})OR^{8a}, —N(R^{8a})S(O)₂R^{8a}, —N(R^{8a})C(O)R^{8a}, —N(R^{8a})N(R^{8a})₂, —N(R^{8a})C(O)OR^{8a}, —N(R^{8a})C(O)N(R^{8a})₂, —S(O)₂R^{8a}, —S(O)R^{8a}, —S(O)N(R^{8a})₂, —S(O)₂N(R^{8a})₂, —N⁺(R^{8a})₃, —S⁺(R^{8a})_{2,} or —Si(R^{8a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁸⁵;
R⁹ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{9a}, —C(S)R^{9a}, —C(O)OR^{9a}, —C(S)SR^{9a}, —C(O)SR^{9a}, —C(S)OR^{9a}, —SC(O)R^{9a}, —OC(S)R^{9a}, —SC(S)R^{9a}, —C(O)N(R^{9a})₂, —OR^{9a}, —SR^{9a}, —N(R^{9a})₂, —N(R^{9a})OR^{9a}, —N(R^{9a})S(O)₂R^{9a}, —N(R^{9a})C(O)R^{9a}, —N(R^{9a})N(R^{9a})₂, —N(R^{9a})C(O)OR^{9a}, —N(R^{9a})C(O)N(R^{9a})₂, —S(O)₂R^{9a}, —S(O)R^{9a}, —S(O)N(R^{9a})₂, —S(O)₂N(R^{9a})₂, —N⁺(R^{9a})₃, —S⁺(R^{9a})_{2,} or —Si(R^{9a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁹⁵;
R¹¹ is —C(O)R^{11a}, —CN, —NHCOH and —NHS(O)₂CH₃, wherein R^{11a} is selected from the group consisting of —OR¹¹⁵, —N(OH)R¹¹⁵, —CH₂OH, —NHNH₂, —N(R¹¹⁵)OR¹¹⁵, —NHR¹¹⁵ and —ONHR¹¹⁵; and wherein R¹¹⁵, in each occurrence, is independently H or C₁₋₄alkyl;
Z is C(R¹⁰)₂, wherein R¹⁰ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{10a}, —C(S)R^{10a}, —C(O)OR^{10a}, —C(S)SR^{10a}, —C(O)SR^{10a}, —C(S)OR^{10a}, —SC(O)R^{10a}, —OC(S)R^{10a}, —SC(S)R^{10a}, —C(O)N(R^{10a})₂, —OR^{10a}, —SR^{10a}, —N(R^{10a})₂, —N(R^{10a})OR^{10a}, —N(R^{10a})S(O)₂R^{10a}, —N(R^{10a})C(O)R^{10a}, —N(R^{10a})N(R^{10a})₂, —N(R^{10a})C(O)OR^{10a}, —N(R^{10a})C(O)N(R^{10a})₂, —S(O)₂R^{10a}, —S(O)R^{10a}, —S(O)N(R^{10a})₂, —S(O)₂N(R^{10a})₂, —N⁺(R^{10a})₃, —S⁺(R^{10a})₂, or —Si(R^{10a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R¹⁰⁵
R^{1a}, R^{2a}, R^{3a}, R^{4a}, R^{5a}, R^{6a}, R^{7a}, R^{8a}, R^{9a}, and R^{10a}, in each occurrence, are independently H, C₁₋₁₂alkyl, C₂₋₁₂alkenyl, C₂₋₁₂alkynyl, C₁₋₁₂alkoxy, C₁₋₁₂acyl, —Si(C₁₋₁₂alkyl)₃, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, C₁₋₁₂acyl, 3 to 12-membered carbocyclyl, and 3 to 12-membered heterocyclyl are each optionally substituted with one or more R¹⁷;
R¹⁵, R²⁵, R³⁵, R⁴⁵, R⁵⁵, R⁶⁵, R⁷⁵, R⁸⁵, R⁹⁵, and R¹⁰⁵, in each occurrence, are independently halo, —OH, —CN, C₁₋₁₂alkyl, C₂₋₁₂alkenyl, C₂₋₁₂alkynyl, C₁₋₁₂alkoxy, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, 3 to 12-membered carbocyclyl, and 3 to 12-membered heterocyclyl are each optionally substituted with one or more R¹⁹; and
R¹⁷ and R¹⁹, in each occurrence, are independently halo, —OH, —CN, C₁₋₁₂alkyl, C₂₋₁₂alkenyl, C₂₋₁₂alkynyl, C₁₋₁₂alkoxy, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, 3 to 12-membered carbocyclyl and 3 to 12-membered heterocyclyl are each optionally substituted with one or more groups independently selected from halo, —OH, and C₁₋₄alkoxy;
m is 0 or 1,
n is 1 or 2;
p is 0 or an integer from 1 to 8;
q is 0 or an integer from 1 to 3; and
s is an integer from 1 to 3.

An alternative first embodiment of the invention is a compound of Formula I:

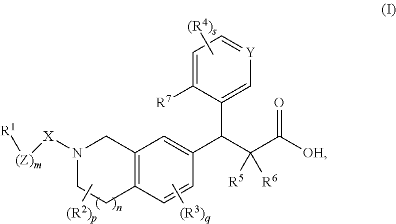

or a pharmaceutically acceptable salt thereof, wherein
R¹ is a 3 to 12-membered carbocyclyl, a 3 to 12-membered heterocyclyl, or —N(R^{1a})₂, wherein the 3 to 12-membered carbocyclyl and 3 to 12-membered heterocyclyl are each optionally substituted with one or more R¹⁵;
X is —C(O)— or —S(O)₂—;
R² is halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{2a}, —C(S)R^{2a}, —C(O)OR^{2a}, —C(S)SR^{2a}, —C(O)SR^{2a}, —C(S)OR^{2a}, —SC(O)R^{2a}, —OC(S)R^{2a}, —SC(S)R^{2a}, —C(O)N(R^{2a})₂, —OR^{2a}, —SR^{2a}, —N(R^{2a})₂, —N(R^{2a})OR^{2a}, —N(R^{2a})S(O)₂R^{2a}, —N(R^{2a})C(O)R^{2a}, —N(R^{2a})N(R^{2a})₂, —N(R^{2a})C(O)OR^{2a}, —N(R^{2a})C(O)N(R^{2a})₂, —S(O)₂R^{2a}, —S(O)R^{2a}, —S(O)N(R^{2a})₂, —S(O)₂N(R^{2a})₂, —N⁺(R^{2a})₃, —S⁺(R^{2a})₂, or —Si(R^{2a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R²⁵;
R³ is halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{3a}, —C(S)R^{3a}, —C(O)OR^{3a}, —C(S)SR^{3a}, —C(O)SR^{3a}, —C(S)OR^{3a}, —SC(O)R^{3a}, —OC(S)R^{3a}, —SC(S)R^{3a}, —C(O)N(R^{3a})₂, —OR^{3a}, —SR^{3a}, —N(R^{3a})₂, —N(R^{3a})OR^{3a}, —N(R^{3a})S(O)₂R^{3a}, —N(R^{3a})C(O)R^{3a}, —N(R^{3a})N(R^{3a})₂, —N(R^{3a})C(O)OR^{3a}, —N(R^{3a})C(O)N(R^{3a})₂, —S(O)₂R^{3a}, —S(O)R^{3a}, —S(O)N(R^{3a})₂, —S(O)₂N(R^{3a})₂, —N⁺(R^{3a})₃, —S⁺(R^{3a})₂, or —Si(R^{3a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R³⁵;
R⁴ is halo, —NO₂, —CN, —N₃, C₁₋₁₂alkyl, C₂₋₁₂alkenyl, C₂₋₁₂alkynyl, —C(O)R^{4a}, —C(S)R^{4a}, —C(O)OR^{4a}, —C(S)SR^{4a}, —C(O)SR^{4a}, —C(S)OR^{4a}, —SC(O)R^{4a}, —OC(S)R^{4a}, —SC(S)R^{4a}, —C(O)N(R^{4a})₂, —OR^{4a}, —SR^{4a}, —N(R^{4a})₂, —N(R^{4a})OR^{4a}, —N(R^{4a})S(O)₂R^{4a}, —N(R^{4a})C(O)R^{4a}, —N(R^{4a})N(R^{4a})₂, —N(R^{4a})C(O)OR^{4a}, —N(R^{4a})C(O)N(R^{4a})₂, —S(O)₂R^{4a}, —S(O)R^{4a}, —S(O)N(R^{4a})₂, —S(O)₂N(R^{4a})₂, —N⁺(R^{4a})₃, —S⁺(R^{4a})₂, or —Si(R^{4a})₃; or two R⁴ groups, attached to adjacent ring carbon atoms and taken together with the two adjacent ring carbon atoms, form triazolyl, wherein the C₁₋₁₂alkyl, C₂₋₁₂alkenyl, and C₂₋₁₂alkynyl are each optionally substituted with one or more R⁴⁵, and wherein the triazolyl is optionally substituted with one or more R⁹;
R⁵ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{5a}, —C(S)R^{5a}, —C(O)OR^{5a}, —C(S)SR^{5a}, —C(O)SR^{5a}, —C(S)OR^{5a}, —SC(O)R^{5a}, —OC(S)R^{5a}, —SC(S)R^{5a}, —C(O)N(R^{5a})₂, —OR^{5a}, —SR^{5a}, —N(R^{5a})₂, —N(R^{5a})OR^{5a}, —N(R^{5a})S(O)₂R^{5a}, —N(R^{5a})C(O)R^{5a}, —N(R^{5a})N(R^{5a})₂, —N(R^{5a})C(O)OR^{5a}, —N(R^{5a})C(O)N(R^{5a})₂, —S(O)₂R^{5a}, —S(O)R^{5a}, —S(O)N(R^{5a})₂, —S(O)₂N(R^{5a})₂, —N⁺(R^{5a})₃, —S⁺(R^{5a})₂, or —Si(R^{5a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁵⁵;
R⁶, in each occurrence, is independently H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{6a}, —C(S)R^{6a}, —C(O)OR^{6a}, —C(S)SR^{6a}, —C(O)SR^{6a}, —C(S)OR^{6a}, —SC(O)R^{6a}, —OC(S)R^{6a}, —SC(S)R^{6a}, —C(O)N(R^{6a})₂, —OR^{6a}, —SR^{6a}, —N(R^{6a})₂, —N(R^{6a})OR^{6a}, —N(R^{6a})S (O)₂R^{6a}, —N(R^{6a})C(O)R^{6a}, —N(R^{6a})N(R^{6a})₂, —N(R^{6a})C(O)OR^{6a}, —N(R^{6a})C(O)N(R^{6a})₂, —S(O)₂R^{6a}, —S(O)R^{6a}, —S(O)N(R^{6a})₂, —S(O)₂N(R^{6a})₂, —N⁺(R^{6a})₃, —S⁺(R^{6a})_{2,} or —Si(R^{6a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁶⁵;
R⁷ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{7a}, —C(S)R^{7a}, —C(O)OR^{7a}, —C(S)SR^{7a}, —C(O)SR^{7a}, —C(S)OR^{7a}, —SC(O)R^{7a}, —OC(S)R^{7a}, —SC(S)R^{7a}, —C(O)N(R^{7a})₂, —OR^{7a}, —SR^{7a}, —N(R^{7a})₂, —N(R^{7a})OR^{7a}, —N(R^{7a})S(O)₂R^{7a}, —N(R^{7a})C(O)R^{7a}, —N(R^{7a})N(R^{7a})₂, —N(R^{7a})C(O)OR^{7a}, —N(R^{7a})C(O)N(R^{7a})₂, —S(O)₂R^{7a}, —S(O)R^{7a}, —S(O)N(R^{7a})₂, —S(O)₂N(R^{7a})₂, —N⁺(R^{7a})₃, —S⁺(R^{7a})_{2,} or —Si(R^{7a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁷⁵;
Y is N or CR⁸, wherein R⁸ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{8a}, —C(S)R^{8a}, —C(O)OR^{8a}, —C(S)SR^{8a}, —C(O)SR^{8a}, —C(S)OR^{8a}, —SC(O)R^{8a}, —OC(S)R^{8a}, —SC(S)R^{8a}, —C(O)N(R^{8a})₂, —OR^{8a}, —SR^{8a}, —N(R^{8a})₂, —N(R^{8a})OR^{8a}, —N(R^{8a})S(O)₂R^{8a}, —N(R^{8a})C(O)R^{8a}, —N(R^{8a})N(R^{8a})₂, —N(R^{8a})C(O)OR^{8a}, —N(R^{8a})C(O)N(R^{8a})₂, —S(O)₂R^{8a}, —S(O)R^{8a}, —S(O)N(R^{8a})₂, —S(O)₂N(R^{8a})₂, —N⁺(R^{8a})₃, —S⁺(R^{8a})_{2,} or —Si(R^{8a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁸⁵;
R⁹ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{9a}, —C(S)R^{9a}, —C(O)OR^{9a}, —C(S)SR^{9a}, —C(O)SR^{9a}, —C(S)OR^{9a}, —SC(O)R^{9a}, —OC(S)R^{9a}, —SC(S)R^{9a}, —C(O)N(R^{9a})₂, —OR^{9a}, —SR^{9a}, —N(R^{9a})₂, —N(R^{9a})OR^{9a}, —N(R^{9a})S(O)₂R^{9a}, —N(R^{9a})C(O)R^{9a}, —N(R^{9a})N(R^{9a})₂, —N(R^{9a})C(O)OR^{9a}, —N(R^{9a})C(O)N(R^{9a})₂, —S(O)₂R^{9a}, —S(O)R^{9a}, —S(O)N(R^{9a})₂, —S(O)₂N(R^{9a})₂, —N⁺(R^{9a})₃, —S⁺(R^{9a})_{2,} or —Si(R^{9a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁹⁵;
Z is C(R¹⁰)₂, wherein R¹⁰ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{10a}, —C(S)R^{10a}, —C(O)OR^{10a}, —C(S)SR^{10a}, —C(O)SR^{10a}, —C(S)OR^{10a}, —SC(O)R^{10a}, —OC(S)R^{10a}, —SC(S)R^{10a}, —C(O)N(R^{10a})₂, —OR^{10a}, —SR^{10a}, —N(R^{10a})₂, —N(R^{10a})OR^{10a}, —N(R^{10a})S(O)₂R^{10a}, —N(R^{10a})C(O)R^{10a}, —N(R^{10a})N(R^{10a})₂, —N(R^{10a})C(O)OR^{10a}, —N(R^{10a})C(O)N(R^{10a})₂, —S(O)₂R^{10a}, —S(O)R^{10a}, —S(O)N(R^{10a})₂, —S(O)₂N(R^{10a})₂, —N⁺(R^{10a})₃, —S⁺(R^{10a})₂, or —Si(R^{10a})₃, wherein the C₂₋₁₂alkenyl, C₂₋₁₂alkynyl, 3 to 12-membered carbocyclyl, and 3 to 12-membered heterocyclyl are each optionally substituted with one or more R¹⁰⁵
R^{1a}, R^{2a}, R^{3a}, R^{4a}, R^{5a}, R^{6a}, R^{7a}, R^{8a}, R^{9a}, and R^{10a}, in each occurrence, are independently H, C₁₋₁₂alkyl, C₂₋₁₂alkenyl, C₂₋₁₂alkynyl, C₁₋₁₂alkoxy, C₁₋₁₂acyl, —Si(C₁₋₁₂alkyl)₃, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, C₁₋₁₂acyl, 3 to 12-membered carbocyclyl, and 3 to 12-membered heterocyclyl are each optionally substituted with one or more R¹⁷;
R¹⁵, R²⁵, R³⁵, R⁴⁵, R⁵⁵, R⁶⁵, R⁷⁵, R⁸⁵, R⁹⁵, and R¹⁰⁵, in each occurrence, are independently halo, —OH, —CN, C₁₋₁₂alkyl, C₂₋₁₂alkenyl, C₂₋₁₂alkynyl, C₁₋₁₂alkoxy, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, 3 to 12-membered carbocyclyl, and 3 to 12-membered heterocyclyl are each optionally substituted with one or more R¹⁹; and
R¹⁷ and R¹⁹, in each occurrence, are independently halo, —OH, —CN, C₁₋₁₂alkyl, C₂₋₁₂alkenyl, C₂₋₁₂alkynyl, C₁₋₁₂alkoxy, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, 3 to 12-membered carbocyclyl and 3 to 12-membered heterocyclyl are each optionally substituted with one or more groups independently selected from halo, —OH, and C₁₋₄alkoxy;
m is 0 or 1,
n is 1 or 2;
p is 0 or an integer from 1 to 8;
q is 0 or an integer from 1 to 3; and
s is an integer from 1 to 3.

Also provided is a pharmaceutical composition comprising at least one compound described herein, or a pharmaceutically acceptable salt thereof, and at least one pharmaceutically acceptable excipient.

Also provided is a method for activating Nrf2 in a subject in need thereof, the method comprising administering to the subject a therapeutically effective amount of a compound described herein, or a pharmaceutically acceptable salt thereof, thereby activating Nrf2 in the subject.

Also provided is a method for treating a disease caused by oxidative stress in a subject, the method comprising administering to the subject a therapeutically effective amount of a compound described herein, or a pharmaceutically acceptable salt thereof.

Also provided is a method for treating a disorder in a subject, wherein the disorder is selected from the group consisting of a neurodegenerative disease, inflammation/an inflammatory disease, an autoimmune disease, an ischemic fibrotic disease, a cancer, premature aging, a cardiovascular disease, a liver disease, a hemoglobinopathy, thalassemia (e.g., beta-thalassemia), and a metabolic disorder, the method comprising administering to the subject a therapeutically effective amount of a compound described herein, or a pharmaceutically acceptable salt thereof.

Other features or advantages will be apparent from the following detailed description of several embodiments, and also from the appended claims.

### BRIEF DESCRIPTION OF THE DRAWINGS

FIG. 1A shows transcription of GCLC and HMOX in human astrocytes treated with increasing concentrations of Compound 47-Ent1 for 20 hours. FIG. 1B shows transcription of OSGIN1 and NQO1 in human astrocytes treated with increasing concentrations of Compound 47-Ent1 for 20 hours. The x-axis represents log (molar concentrations of compound 47-Ent1.

FIG. 2 shows levels of intracellular glutathione Compound 47-Ent1 in human astrocytes treated with increasing concentrations of Compound 47-Ent1 for 20 hours. The x-axis represents log (molar concentrations of compound 47-Ent1). Values shown are means of triplicate determination in one experiment.

FIG. 3 shows levels of protection of astrocytes by increasing concentrations of Compound 47-Ent1 from oxidative stress-induced cell death caused by 25 μM sodium arsenite. The compound was added to human astrocytes 20 hrs prior to addition of arsenite and the astrocytes were further incubated for 22 hours after addition of arsenite. The x-axis represents log (molar concentrations of compound 47-Ent1). This figure shows mean and standard deviation of triplicate determination in one experiment.

FIGS. 4A to 4D show the expression of Cbr38 (FIG. 4A), Nqo1 (FIG. 4B), Hmox1 (FIG. 4C) and Osgin1 (FIG. 4D) in kidney, isolated from wild-type mice at 2 hours and 6 hours after being treated with Compound 47-Ent1 (10 or 50 mg/kg) or a vehicle.

FIGS. 5A and 5B show the expression of Osgin1 (FIG. 5A) and Nqo1 (FIG. 5B), in brain, isolated from wild-type mice at 2 hours and 6 hours after being treated with Compound 47-Ent1 (10 or 50 mg/kg) or a vehicle.

### DETAILED DESCRIPTION

The compounds or pharmaceutically acceptable salts thereof as described herein are Nrf2 activators.

In a second embodiment of the invention, the compound is represented by Formula A or I, or a pharmaceutically acceptable salt thereof, wherein p and q are each independently 0 or 1; and wherein the values of the other variables are as defined for the first or alternative first embodiment.

In a third embodiment of the invention, the compound is represented by Formula II:

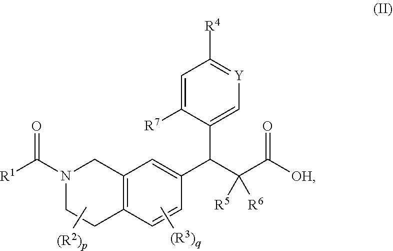

or a pharmaceutically acceptable salt thereof, wherein the values of the variables are as defined for the first, alternative first and/or second embodiments.

In a fourth embodiment of the invention, the compound is represented by Formula IIA or IIB:

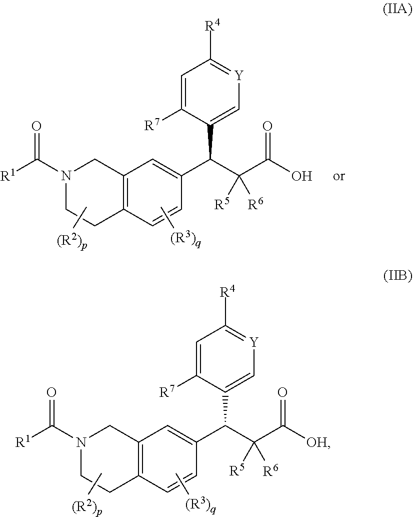

or a pharmaceutically acceptable salt thereof, wherein the values of the variables are as defined for the first, alternative first and/or second embodiments.

In a fifth embodiment of the invention, the compound is represented by Formula A, I, II, II(A), or II(B), or a pharmaceutically acceptable salt thereof, wherein R⁴ is —CN, —C(O)N(R^{4a})₂, or —OR^{4a}; and R^{4a}, in each occurrence, is independently H or C₁₋₆alkyl, wherein the C₁₋₆alkyl is optionally substituted with one to six groups independently selected from halo, —CN, —OH, C₁₋₄alkyl, and C₁₋₄alkoxy; and wherein the values of the other variables are as defined for the first, alternative first and/or second embodiments.

In a sixth embodiment of the invention, the compound is represented by Formula A, I, II, II(A), or II(B), or a pharmaceutically acceptable salt thereof, wherein R⁴ is —CN, —C(O)N(R^{4a})₂, or —OR^{4a}, wherein R^{4a}, in each occurrence, is independently H or C₁₋₄alkyl; and wherein the values of the other variables are as defined for the first, alternative first and/or second embodiments.

In a seventh embodiment of the invention, the compound is represented by Formula III:

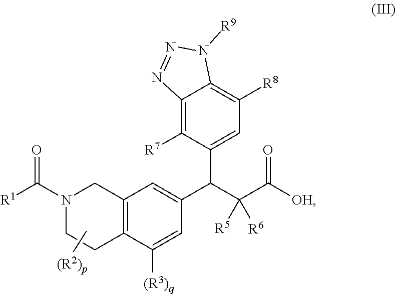

or a pharmaceutically acceptable salt thereof, wherein the values of the variables are as defined for the first, alternative first and/or second embodiments.

In an eighth embodiment of the invention, the compound is represented by Formula IIIA or IIIB:

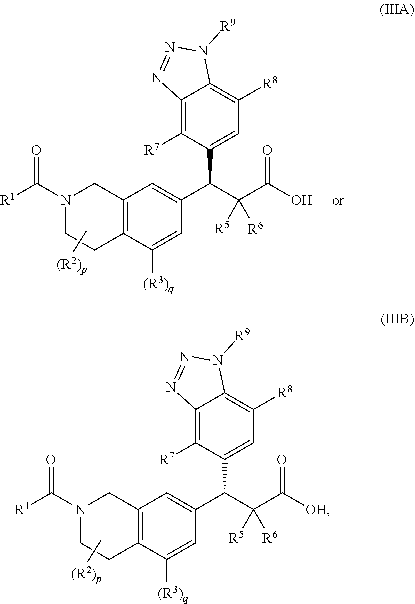

or a pharmaceutically acceptable salt thereof, wherein the values of the variables are as defined for the first, alternative first and/or second embodiments.

In a ninth embodiment of the invention, the compound is represented by Formula IV:

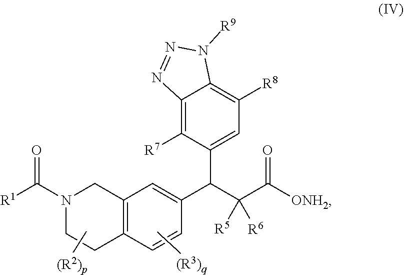

or a pharmaceutically acceptable salt thereof, wherein the values of the variables are as defined for the first, alternative first and/or second embodiments.

In a tenth embodiment of the invention, the compound is represented by Formula A, I, III, III(A), III(B) or IV, or a pharmaceutically acceptable salt thereof, wherein R⁹ is H, halo, —CN, —OR^{9a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R⁹⁵; R^{9a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷; R⁹⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to six groups independently selected from halo, —OH, and C₁₋₄alkoxy; and R¹⁷, in each occurrence, as an optional substituent of R^{9a}, is independently selected from halo, —CN, —OH, C₁₋₆alkyl, and C₁₋₆alkoxy, wherein the C₁₋₆alkyl and C₁₋₆alkoxy are each optionally substituted with one to six halo; and wherein the values of the other variables are as defined for the first, alternative first and/or second embodiments.

In a eleventh embodiment of the invention, the compound is represented by Formula A, I, III, III(A), III(B) or IV, or a pharmaceutically acceptable salt thereof, wherein R⁹ is H, halo, —OH, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy; and wherein the values of the other variables are as defined for the first, alternative first and/or second embodiments.

In an twelfth embodiment of the invention, the compound is represented by Formula A, I, III, III(A), III(B) or IV, or a pharmaceutically acceptable salt thereof, wherein R⁹ is H or C₁₋₄alkyl; and wherein the values of the other variables are as defined for the first, alternative first and/or second embodiments.

In a thirteenth embodiment of the invention, the compound is represented by Formula A, I, II, II(A), II(B), III, III(A), III(B) or IV, or a pharmaceutically acceptable salt thereof, wherein R¹ is a 6 to 11-membered carbocyclyl, a 5 to 10-membered heterocyclyl, or —N(R^{1a})₂, wherein the 6 to 11-membered carbocyclyl and 5 to 10-membered heterocyclyl are each optionally substituted with one to eight R¹⁵; R^{1a}, in each occurrence, is independently selected from H, C₁₋₁₂alkyl, C₂₋₁₂alkenyl, C₂₋₁₂alkynyl, and a 6 to 10-membered aromatic carbocyclyl, wherein the C₁₋₁₂alkyl, C₂₋₁₂alkenyl, C₂₋₁₂alkynyl, and 6 to 10-membered carbocyclyl are each optionally substituted with one to six R¹⁷; R¹⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to six groups independently selected from halo, —OH, and C₁₋₄alkoxy; and R¹⁷, in each occurrence, as an optional substituent of R^{1a}, is independently selected from halo, —CN, —OH, C₁₋₆alkyl, and C₁₋₆alkoxy, wherein the C₁₋₆alkyl and C₁₋₆alkoxy are each optionally substituted with one to six halo; and wherein the values of the other variables are as defined for the first, alternative first, second, fifth, sixth, tenth, eleventh and/or twelfth embodiments.

In a fourteenth embodiment of the invention, the compound is represented by Formula A, I, II, II(A), II(B), III, III(A), III(B) or IV, or a pharmaceutically acceptable salt thereof, wherein R¹ is benzofuran-2-yl, oxazolyl, pyrazolo [1,5-a]pyridine-2-yl, cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, thiazolyl or —N(R^{1a})₂, wherein the benzofuran-2-yl, oxazolyl, pyrazolo [1,5-a]pyridine-2-yl, cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, and thiazolyl are each optionally substituted with one to six groups independently selected from halo, —CN, —OH, C₁₋₄alkyl, and C₁₋₄alkoxy, wherein C₁₋₄alkyl and C₁₋₄alkoxy are optionally substituted with one to six halo; and wherein the values of the other variables are as defined for the first, alternative first, second, fifth, sixth, tenth, eleventh and/or twelfth embodiments.

In a fifteenth embodiment of the invention, the compound is represented by Formula A, I, II, II(A), II(B), III, III(A), III(B) or IV, or a pharmaceutically acceptable salt thereof, wherein R¹ is cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, thiazolyl or —N(R^{1a})₂, wherein the cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, and thiazolyl are each optionally substituted with one to six groups independently selected from halo, —CN, —OH, C₁₋₄alkyl, and C₁₋₄alkoxy, wherein C₁₋₄alkyl and C₁₋₄alkoxy are optionally substituted with one to six halo; and R^{1a}, in each occurrence, is independently C₁₋₄alkyl or phenyl; and wherein the values of the other variables are as defined for the first, alternative first, second, fifth, sixth, tenth, eleventh and/or twelfth embodiments.

In a sixteenth embodiment of the invention, the compound is represented by Formula A, I, II, II(A), II(B), III, III(A), III(B) or IV, or a pharmaceutically acceptable salt thereof, wherein R² is halo, —CN, —OR^{2a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R²⁵; R^{2a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷; R²⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to six groups independently selected from halo, —OH, and C₁₋₄alkoxy; and R¹⁷, in each occurrence, as an optional substituent of R^{2a}, is independently selected from halo, —CN, —OH, C₁₋₆alkyl, and C₁₋₆alkoxy, wherein the C₁₋₆alkyl and C₁₋₆alkoxy are each optionally substituted with one to six halo; and wherein the values of the other variables are as defined for the first, alternative first, second, fifth, sixth, tenth, eleventh, twelfth, thirteenth, fourteenth and/or fifteenth embodiments.

In a seventeenth embodiment of the invention, the compound is represented by Formula A, I, II, II(A), II(B), III, III(A), III(B) or IV, or a pharmaceutically acceptable salt thereof, wherein R² is halo, —OH, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy; and wherein the values of the other variables are as defined for the first, alternative first, second, fifth, sixth, tenth, eleventh, twelfth, thirteenth, fourteenth and/or fifteenth embodiments.

In a eighteenth embodiment of the invention, the compound is represented by Formula A, I, II, II(A), II(B), III, III(A), III(B) or IV, or a pharmaceutically acceptable salt thereof, wherein R² is C₁₋₄alkyl; and wherein the values of the other variables are as defined for the first, alternative first, second, fifth, sixth, tenth, eleventh, twelfth, thirteenth, fourteenth and/or fifteenth embodiments.

In a nineteenth embodiment of the invention, the compound is represented by Formula A, I, II, II(A), II(B), III, III(A), III(B) or IV, or a pharmaceutically acceptable salt thereof, wherein R³ is halo, —NO₂, —CN, —OR^{3a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R³⁵; R^{3a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷; R³⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to six groups independently selected from halo, —OH, and C₁₋₄alkoxy; and R¹⁷, in each occurrence, as an optional substituent of R^{3a}, is independently selected from halo, —CN, —OH, C₁₋₆alkyl, and C₁₋₆alkoxy, wherein the C₁₋₆alkyl and C₁₋₆alkoxy are each optionally substituted with one to six halo; and wherein the values of the other variables are as defined for the first, alternative first, second, fifth, sixth, tenth, eleventh, twelfth, thirteenth, fourteenth, fifteenth, sixteenth, seventeenth and/or eighteenth embodiments.

In an twentieth embodiment of the invention, the compound is represented by Formula A, I, II, II(A), II(B), III, III(A), III(B) or IV, or a pharmaceutically acceptable salt thereof, wherein R³ is halo, —OH, —NO₂, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy; and wherein the values of the other variables are as defined for the first, alternative first, second, fifth, sixth, tenth, eleventh, twelfth, thirteenth, fourteenth, fifteenth, sixteenth, seventeenth and/or eighteenth embodiments.

In a twenty-first embodiment of the invention, the compound is represented by Formula A, I, II, II(A), II(B), III, III(A), III(B) or IV, or a pharmaceutically acceptable salt thereof, wherein R³ is C₁₋₄alkyl or —NO₂; and wherein the values of the other variables are as defined for the first, alternative first, second, fifth, sixth, tenth, eleventh, twelfth, thirteenth, fourteenth, fifteenth, sixteenth, seventeenth and/or eighteenth embodiments.

In a twenty-second embodiment of the invention, the compound is represented by Formula A, I, II, II(A), II(B), III, III(A), III(B) or IV, or a pharmaceutically acceptable salt thereof, wherein R⁵ is H, halo, —CN, —OR^{5a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R⁵⁵; R^{5a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷; R⁵⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to eight groups independently selected from halo, —OH, and C₁₋₄alkoxy; and R¹⁷, in each occurrence, as an optional substituent of R^{5a}, is independently selected from halo, —CN, —OH, C₁₋₆alkyl, and C₁₋₆alkoxy, wherein the C₁₋₆alkyl and C₁₋₆alkoxy are each optionally substituted with one to six halo; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth and/or twenty-first embodiments.

In a twenty-third embodiment of the invention, the compound is represented by Formula A, I, II, II(A), II(B), III, III(A), III(B) or IV, or a pharmaceutically acceptable salt thereof, wherein R⁵ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth and/or twenty-first embodiments.

In a twenty-fourth embodiment of the invention, the compound is represented by Formula A, I, II, II(A), II(B), III, III(A), III(B) or IV, or a pharmaceutically acceptable salt thereof, wherein R⁵ is H or C₁₋₄alkyl; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth and/or twenty-first embodiments.

In a twenty-fifth embodiment of the invention, the compound is represented by Formula A, I, II, II(A), II(B), III, III(A), III(B) or IV, or a pharmaceutically acceptable salt thereof, wherein R⁶ is H, halo, —CN, —OR^{6a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R⁶⁵; R^{6a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷; R⁶⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to eight groups independently selected from halo, —OH, and C₁₋₄alkoxy; and R¹⁷, in each occurrence, as an optional substituent of R^{6a}, is independently selected from halo, —CN, —OH, C₁₋₆alkyl, and C₁₋₆alkoxy, wherein the C₁₋₆alkyl and C₁₋₆alkoxy are each optionally substituted with one to six halo; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth, twenty-first, twenty-second, twenty-third and/or twenty-fourth embodiments.

In a twenty-sixth embodiment of the invention, the compound is represented by Formula A, I, II, II(A), II(B), III, III(A), III(B) or IV, or a pharmaceutically acceptable salt thereof, wherein R⁶ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth, twenty-first, twenty-second, twenty-third and/or twenty-fourth embodiments.

In a twenty-seventh embodiment of the invention, the compound is represented by Formula A, I, II, II(A), II(B), III, III(A), III(B) or IV, or a pharmaceutically acceptable salt thereof, wherein R⁶ is H or C₁₋₄alkyl; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth, twenty-first, twenty-second, twenty-third and/or twenty-fourth embodiments.

In a twenty-eighth embodiment of the invention, the compound is represented by Formula A, I, II, II(A), II(B), III, III(A), III(B) or IV, or a pharmaceutically acceptable salt thereof, wherein R⁷ is H, halo, —CN, —OR^{7a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R⁷⁵; R^{7a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷; R⁷⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to eight groups independently selected from halo, —OH, and C₁₋₄alkoxy; and R¹⁷, in each occurrence, as an optional substituent of R^{7a}, is independently selected from halo, —CN, —OH, C₁₋₆alkyl, and C₁₋₆alkoxy, wherein the C₁₋₆alkyl and C₁₋₆alkoxy are each optionally substituted with one to six halo; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth and/or twenty-seventh embodiments.

In a twenty-nineth embodiment of the invention, the compound is represented by Formula A, I, II, II(A), II(B), III, III(A), III(B) or IV, or a pharmaceutically acceptable salt thereof, wherein R⁷ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth and/or twenty-seventh embodiments.

In a thirtieth embodiment of the invention, the compound is represented by Formula A, I, II, II(A), II(B), III, III(A), III(B) or IV, or a pharmaceutically acceptable salt thereof, wherein R⁷ is H or C₁₋₄alkyl; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth and/or twenty-seventh embodiments.

In a thirty-first embodiment of the invention, the compound is represented by Formula A, I, II, II(A), II(B), III, III(A), III(B) or IV, or a pharmaceutically acceptable salt thereof, wherein R⁸ is H, halo, —CN, —OR^{8a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R⁸⁵; R^{8a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷; R⁸⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to six groups independently selected from halo, —OH, and C₁₋₄alkoxy; and R¹⁷, in each occurrence, as an optional substituent of R^{8a}, is independently selected from halo, —CN, —OH, C₁₋₆alkyl, and C₁₋₆alkoxy, wherein the C₁₋₆alkyl and C₁₋₆alkoxy are each optionally substituted with one to six halo; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-nineth and/or thirtieth embodiments.

In a thirty-second embodiment of the invention, the compound is represented by Formula A, I, II, II(A), II(B), III, III(A), III(B) or IV, or a pharmaceutically acceptable salt thereof, wherein R⁸ is H, halo, —OH, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-nineth and/or thirtieth embodiments.

In a thirty-third embodiment of the invention, the compound is represented by Formula A, I, II, II(A), II(B), III, III(A), III(B) or IV, or a pharmaceutically acceptable salt thereof, wherein R⁸ is H or C₁₋₄alkyl; and wherein the values of the other variables are as defined for the first, alternative first, second, fifth, sixth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-nineth and/or thirtieth embodiments.

In a thirty-fourth embodiment of the invention, the compound is represented by Formula A, I, III, III(A), III(B) or IV, or a pharmaceutically acceptable salt thereof, wherein R¹ is a 6 to 11-membered carbocyclyl, a 5 to 10-membered heterocyclyl, or —N(R^{1a})₂, wherein the 6 to 11-membered carbocyclyl and 5 to 10-membered heterocyclyl are each optionally substituted with one to six groups selected from halo, —CN, —OH, C₁₋₆alkyl, and C₁₋₆alkoxy, wherein the C₁₋₆alkyl and C₁₋₆alkoxy, in each occurrence, are optionally substituted with one to six halo; and wherein R^{1a}, in each occurrence, is independently selected from C₁₋₆alkyl and a 6 to 10-membered aromatic carbocyclyl, wherein the C₁₋₆alkyl and 6 to 10-membered carbocyclyl are each optionally substituted with one to six groups selected from halo, —CN, —OH, C₁₋₄alkyl, and C₁₋₄alkoxy; X is —C(O)—; R² is halo, —OH, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy; R³ is halo, —OH, —NO₂, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy; two R⁴ groups, attached to adjacent ring carbon atoms and taken together with the two adjacent ring carbon atoms, form triazolyl, wherein the triazolyl is optionally substituted with R⁹; R⁵ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy; R⁶ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy; R⁷ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy; Y is CR⁸; R⁸ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy; R⁹ is H, halo, —OH, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy; m is 0; n is 1; p is 0 or 1; q is 0 or 1; and s is 2.

In a thirty-fifth embodiment of the invention, the compound is represented by Formula A, I, III, III(A), III(B) or IV, or a pharmaceutically acceptable salt thereof, wherein R¹ is cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, thiazolyl or —N(R^{1a})₂, wherein the cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, and thiazolyl are each optionally substituted with one to six groups independently selected from halo, —CN, —OH, C₁₋₄alkyl, and C₁₋₄alkoxy, wherein C₁₋₄alkyl, and C₁₋₄alkoxy are optionally substituted with one to six halo; and wherein R^{1a}, in each occurrence, is independently C₁₋₄alkyl or phenyl; X is —C(O)—; R² is C₁₋₄alkyl, wherein the C₁₋₄alkyl is optionally substituted with one to three groups independently selected from halo, —OH, and C₁₋₄alkoxy; R³ is —NO₂ or C₁₋₄alkyl, wherein the C₁₋₄alkyl is optionally substituted with one to three groups independently selected from halo, —OH, and C₁₋₄alkoxy; two R⁴ groups, attached to adjacent ring carbon atoms and taken together with the two adjacent ring carbon atoms, form triazolyl, wherein the triazolyl is optionally substituted with R⁹; R⁵ is H or C₁₋₄alkyl, wherein the C₁₋₄alkyl is optionally substituted by one to three groups independently selected from halo, —OH, and C₁₋₄alkoxy; R⁶ is H or C₁₋₄alkyl, wherein the C₁₋₄alkyl is optionally substituted by one to three groups independently selected from halo, —OH, and C₁₋₄alkoxy; R⁷ is H or C₁₋₄alkyl, wherein the C₁₋₄alkyl is optionally substituted by one to three groups independently selected from halo, —OH, and C₁₋₄alkoxy; Y is CR⁸; R⁸ is H or C₁₋₄alkyl, wherein the C₁₋₄alkyl is optionally substituted by one to three groups independently selected from halo, —OH, and C₁₋₄alkoxy; R⁹ is H or C₁₋₄alkyl; m is 0; n is 1; p is 0 or 1; q is 0 or 1; and s is 2.

In a thirty-sixth embodiment of the invention, the compound is represented by Formula A, I, III, III(A), III(B) or IV, or a pharmaceutically acceptable salt thereof, wherein R¹ is cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, thiazolyl or —N(R^{1a})₂, wherein the cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, and thiazolyl are each optionally substituted with one to six groups selected from halo, —CN, —OH, methyl, isopropyl, t-butyl, and methoxy, wherein the methyl, isopropyl, t-butyl, and methoxy, in each occurrence, are optionally substituted with one to three halo, and wherein one R^{1a} is C₁₋₄alkyl and the other is phenyl; X is —C(O)—; R² is C₁₋₄alkyl; R³ is C₁₋₄alkyl or —NO₂; two R⁴ groups, attached to adjacent ring carbon atoms and taken together with the two adjacent ring carbon atoms, form triazolyl, wherein the triazolyl is optionally substituted with R⁹; R⁵ is H or C₁₋₄alkyl; R⁶ is H or C₁₋₄alkyl; R⁷ is H or C₁₋₄alkyl; Y is CR⁸; R⁸ is H; R⁹ is C₁₋₄alkyl; m is 0; n is 1; p is 0 or 1; q is 0 or 1; and s is 2.

In a thirty-seventh embodiment of the invention, the compound is represented by Formula A, I, III, III(A), III(B) or IV, or a pharmaceutically acceptable salt thereof, wherein R¹ is phenyl, wherein the phenyl is optionally substituted with one to four groups selected from methyl and fluoro; X is —C(O)—; R² is methyl; R³ is methyl; two R⁴ groups, attached to adjacent ring carbon atoms and taken together with the two adjacent ring carbon atoms, form triazolyl, wherein the triazolyl is optionally substituted with methyl or ethyl; R⁵ is H or methyl; R⁶ is H or methyl; R⁷ is H or methyl; Y is CR⁸; R⁸ is H; m is 0; n is 1; p is 0 or 1; q is 0 or 1; and s is 2.

In a thirty-eighth embodiment of the invention, the compound is selected from the group consisting of:
3-(1-ethyl-4-methyl-benzotriazol-5-yl)-3-[2-(4-methylbenzoyl)-3,4-dihydro-1H-isoquinolin-7-yl]propanoic acid;
3-(1-ethyl-4-methyl-benzotriazol-5-yl)-3-[2-(4-methoxybenzoyl)-3,4-dihydro-1H-isoquinolin-7-yl]propanoic acid;
3-(1-ethyl-4-methyl-benzotriazol-5-yl)-3-[2-(4-hydroxybenzoyl)-3,4-dihydro-1H-isoquinolin-7-yl]propanoic acid;
3-[2-(4-chlorobenzoyl)-3,4-dihydro-1H-isoquinolin-7-yl]-3-(1-ethyl-4-methyl-benzotriazol-5-yl)propanoic acid;
3-[2-(4-tert-butylbenzoyl)-3,4-dihydro-1H-isoquinolin-7-yl]-3-(1-ethyl-4-methyl-benzotriazol-5-yl)propanoic acid;
3-[2-(cyclohexanecarbonyl)-3,4-dihydro-1H-isoquinolin-7-yl]-3-(1-ethyl-4-methyl-benzotriazol-5-yl)propanoic acid;
3-[2-(2-chlorobenzoyl)-3,4-dihydro-1H-isoquinolin-7-yl]-3-(1-ethyl-4-methyl-benzotriazol-5-yl)propanoic acid;
3-[2-(2,4-dichlorobenzoyl)-3,4-dihydro-1H-isoquinolin-7-yl]-3-(1-ethyl-4-methyl-benzotriazol-5-yl)propanoic acid;
3-[2-(2,5-dichlorobenzoyl)-3,4-dihydro-1H-isoquinolin-7-yl]-3-(1-ethyl-4-methyl-benzotriazol-5-yl)propanoic acid;
3-[2-(3-chlorobenzoyl)-3,4-dihydro-1H-isoquinolin-7-yl]-3-(1-ethyl-4-methyl-benzotriazol-5-yl)propanoic acid;
3-[2-(3,4-dichlorobenzoyl)-3,4-dihydro-1H-isoquinolin-7-yl]-3-(1-ethyl-4-methyl-benzotriazol-5-yl)propanoic acid;
(3S)-3-[2-(3,4-dichlorobenzoyl)-3,4-dihydro-1H-isoquinolin-7-yl]-3-(1-ethyl-4-methyl-benzotriazol-5-yl)propanoic acid;
(3R)-3-[2-(3,4-dichlorobenzoyl)-3,4-dihydro-1H-isoquinolin-7-yl]-3-(1-ethyl-4-methyl-benzotriazol-5-yl)propanoic acid;
3-[2-(3,5-dichlorobenzoyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-(pyrimidine-2-carbonyl)-3,4-dihydro-1H-isoquinolin-7-yl]propanoic acid;
3-(1-ethyl-4-methyl-benzotriazol-5-yl)-3-[2-(pyrazine-2-carbonyl)-3,4-dihydro-1H-isoquinolin-7-yl]propanoic acid;
3-(1-ethyl-4-methyl-benzotriazol-5-yl)-3-[2-(pyrimidine-4-carbonyl)-3,4-dihydro-1H-isoquinolin-7-yl]propanoic acid;
3-(1-ethyl-4-methyl-benzotriazol-5-yl)-3-[2-(pyridine-2-carbonyl)-3,4-dihydro-1H-isoquinolin-7-yl]propanoic acid;
3-(1-ethyl-4-methyl-benzotriazol-5-yl)-3-[2-(pyridine-3-carbonyl)-3,4-dihydro-1H-isoquinolin-7-yl]propanoic acid;
3-(1-ethyl-4-methyl-benzotriazol-5-yl)-3-[2-(pyridine-4-carbonyl)-3,4-dihydro-1H-isoquinolin-7-yl]propanoic acid;
3-(1-ethyl-4-methyl-benzotriazol-5-yl)-3-[2-(1-methylpyrazole-4-carbonyl)-3,4-dihydro-1H-isoquinolin-7-yl]propanoic acid;
3-(1-ethyl-4-methyl-benzotriazol-5-yl)-3-[2-(1-methylpyrazole-3-carbonyl)-3,4-dihydro-1H-isoquinolin-7-yl]propanoic acid;
3-(1-ethyl-4-methyl-benzotriazol-5-yl)-3-[2-(1-methylimidazole-4-carbonyl)-3,4-dihydro-1H-isoquinolin-7-yl]propanoic acid;
3-(1-ethyl-4-methyl-benzotriazol-5-yl)-3-[2-(isoxazole-3-carbonyl)-3,4-dihydro-1H-isoquinolin-7-yl]propanoic acid;
3-(1-ethyl-4-methyl-benzotriazol-5-yl)-3-[2-(thiazole-4-carbonyl)-3,4-dihydro-1H-isoquinolin-7-yl]propanoic acid;
3-(1-ethyl-4-methyl-benzotriazol-5-yl)-3-[2-(3-methylbenzoyl)-3,4-dihydro-1H-isoquinolin-7-yl]propanoic acid;
3-[2-(3,4-dimethylbenzoyl)-3,4-dihydro-1H-isoquinolin-7-yl]-3-(1-ethyl-4-methyl-benzotriazol-5-yl)propanoic acid;
3-[2-(2,3-dimethylbenzoyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-(2,3,5-trimethylbenzoyl)-3,4-dihydro-1H-isoquinolin-7-yl]propanoic acid;
3-[2-(3,5-diethylbenzoyl)-3,4-dihydro-1H-isoquinolin-7-yl]-3-(1-ethyl-4-methyl-benzotriazol-5-yl)propanoic acid;
3-[2-(3,5-dimethylbenzoyl)-3,4-dihydro-1H-isoquinolin-7-yl]-3-(1-ethyl-4-methyl-benzotriazol-5-yl)propanoic acid;
(3S)-3-[2-(3,5-dimethylbenzoyl)-3,4-dihydro-1H-isoquinolin-7-yl]-3-(1-ethyl-4-methyl-benzotriazol-5-yl)propanoic acid;
(3R)-3-[2-(3,5-dimethylbenzoyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-(2,3,5,6-tetramethylbenzoyl)-3,4-dihydro-1H-isoquinolin-7-yl]propanoic acid;
3-[2-(2,6-dimethylbenzoyl)-3,4-dihydro-1H-isoquinolin-7-yl]-3-(1-ethyl-4-methyl-benzotriazol-5-yl)propanoic acid;
3-[2-(2,6-diethylbenzoyl)-3,4-dihydro-1H-isoquinolin-7-yl]-3-(1-ethyl-4-methyl-benzotriazol-5-yl)propanoic acid;
3-[2-(2,6-difluorobenzoyl)-3,4-dihydro-1H-isoquinolin-7-yl]-3-(1-ethyl-4-methyl-benzotriazol-5-yl)propanoic acid;
3-[2-(adamantane-1-carbonyl)-3,4-dihydro-1H-isoquinolin-7-yl]-3-(1-ethyl-4-methyl-benzotriazol-5-yl)propanoic acid;
3-[2-(bicyclo[2.2.2]octane-4-carbonyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-[4-(trifluoromethyl)cyclohexanecarbonyl]-3,4-dihydro-1H-isoquinolin-7-yl]propanoic acid;
3-(1-ethyl-4-methyl-benzotriazol-5-yl)-3-[2-[4-(trifluoromethyl)cyclohexanecarbonyl]-3,4-dihydro-1H-isoquinolin-7-yl]propanoic acid;
3-(1-ethyl-4-methyl-benzotriazol-5-yl)-3-[2-(2-phenylacetyl)-3,4-dihydro-1H-isoquinolin-7-yl]propanoic acid;
(3S)-3-(1-ethyl-4-methyl-benzotriazol-5-yl)-3-[2-(2-phenylacetyl)-3,4-dihydro-1H-isoquinolin-7-yl]propanoic acid;
(3R)-3-(1-ethyl-4-methyl-benzotriazol-5-yl)-3-[2-(2-phenylacetyl)-3,4-dihydro-1H-isoquinolin-7-yl]propanoic acid;
3-(1-ethyl-4-methyl-benzotriazol-5-yl)-3-[2-[(2S)-2-methoxy-2-phenyl-acetyl]-3,4-dihydro-1H-isoquinolin-7-yl]propanoic acid;
3-(1-ethyl-4-methyl-benzotriazol-5-yl)-3-[2-(piperidine-1-carbonyl)-3,4-dihydro-1H-isoquinolin-7-yl]propanoic acid;
3-(1-ethyl-4-methyl-benzotriazol-5-yl)-3-[2-[4-(trifluoromethyl)piperidine-1-carbonyl]-3,4-dihydro-1H-isoquinolin-7-yl]propanoic acid;
3-(1-ethyl-4-methyl-benzotriazol-5-yl)-3-[2-[methyl(phenyl)carbamoyl]-3,4-dihydro-1H-isoquinolin-7-yl]propanoic acid;
3-(1-ethyl-4-methyl-benzotriazol-5-yl)-3-[2-(2-isopropylmorpholine-4-carbonyl)-3,4-dihydro-1H-isoquinolin-7-yl]propanoic acid;
(3S)-3-(1-ethyl-4-methyl-benzotriazol-5-yl)-3-[2-(2,3,5,6-tetramethylbenzoyl)-3,4-dihydro-1H-isoquinolin-7-yl]propanoic acid;
(3R)-3-(1-ethyl-4-methyl-benzotriazol-5-yl)-3-[2-(2,3,5,6-tetramethylbenzoyl)-3,4-dihydro-1H-isoquinolin-7-yl]propanoic acid;
(3S)-3-(1-ethyl-4-methyl-benzotriazol-5-yl)-3-[2-(2-methylbenzoyl)-3,4-dihydro-1H-isoquinolin-7-yl]propanoic acid;
(3R)-3-(1-ethyl-4-methyl-benzotriazol-5-yl)-3-[2-(2-methylbenzoyl)-3,4-dihydro-1H-isoquinolin-7-yl]propanoic acid;
(3S)-3-(2-benzoyl-3,4-dihydro-1H-isoquinolin-7-yl)-3-(1-ethyl-4-methyl-benzotriazol-5-yl)propanoic acid;
(3R)-3-(2-benzoyl-3,4-dihydro-1H-isoquinolin-7-yl)-3-(1-ethyl-4-methyl-benzotriazol-5-yl)propanoic acid;
3-[2-(benzenesulfonyl)-3,4-dihydro-1H-isoquinolin-7-yl]-3-(1-methylbenzotriazol-5-yl)propanoic acid;
3-(2-benzoyl-3,4-dihydro-1H-isoquinolin-7-yl)-3-(1-methylbenzotriazol-5-yl)propanoic acid;
(3S)-3-(2-benzoyl-3,4-dihydro-1H-isoquinolin-7-yl)-3-(1-methylbenzotriazol-5-yl)propanoic acid;
(3R)-3-(2-benzoyl-3,4-dihydro-1H-isoquinolin-7-yl)-3-(1-methylbenzotriazol-5-yl)propanoic acid;
(3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoic acid;
(3S)-3-(1-ethyl-4-methyl-benzotriazol-5-yl)-2,2-dimethyl-3-[2-(2,3,5,6-tetramethylbenzoyl)-3,4-dihydro-1H-isoquinolin-7-yl]propanoic acid;
(2S)-2-[(S)-(1-ethyl-4-methyl-benzotriazol-5-yl)-[2-(2,3,5,6-tetramethylbenzoyl)-3,4-dihydro-1H-isoquinolin-7-yl]methyl]butanoic acid;
(2R)-2-[(S)-(1-ethyl-4-methyl-benzotriazol-5-yl)-[2-(2,3,5,6-tetramethylbenzoyl)-3,4-dihydro-1H-isoquinolin-7-yl]methyl]butanoic acid;
3-(6-methoxy-4-methyl-3-pyridyl)-3-[2-(2,3,5,6-tetramethylbenzoyl)-3,4-dihydro-1H-isoquinolin-7-yl]propanoic acid;
(3S)-3-(6-methoxy-4-methyl-3-pyridyl)-3-[2-(2,3,5,6-tetramethylbenzoyl)-3,4-dihydro-1H-isoquinolin-7-yl]propanoic acid;
(3R)-3-(6-methoxy-4-methyl-3-pyridyl)-3-[2-(2,3,5,6-tetramethylbenzoyl)-3,4-dihydro-1H-isoquinolin-7-yl]propanoic acid;
3-(4-cyano-2-methyl-phenyl)-3-[2-(2,3,5,6-tetramethylbenzoyl)-3,4-dihydro-1H-isoquinolin-7-yl]propanoic acid;
3-(4-carbamoyl-2-methyl-phenyl)-3-[2-(2,3,5,6-tetramethylbenzoyl)-3,4-dihydro-1H-isoquinolin-7-yl]propanoic acid;
3-(2-benzoyl-3,4-dihydro-1H-isoquinolin-7-yl)-3-(4-cyano-2-methyl-phenyl)propanoic acid;
3-[(1S)-2-benzoyl-1-methyl-3,4-dihydro-1H-isoquinolin-7-yl]-3-(1-ethyl-4-methyl-benzotriazol-5-yl)propanoic acid;
3-(2-benzoyl-1,3,4,5-tetrahydro-2-benzazepin-8-yl)-3-(1-ethyl-4-methyl-benzotriazol-5-yl)propanoic acid;
(3S)-3-[(4S)-2-benzoyl-4-methyl-3,4-dihydro-1H-isoquinolin-7-yl]-3-(1-ethyl-4-methyl-benzotriazol-5-yl)propanoic acid; N-ethylethanamine
(3R)-3-[(4S)-2-benzoyl-4-methyl-3,4-dihydro-1H-isoquinolin-7-yl]-3-(1-ethyl-4-methyl-benzotriazol-5-yl)propanoic acid;
(3S)-3-[(4R)-2-benzoyl-4-methyl-3,4-dihydro-1H-isoquinolin-7-yl]-3-(1-ethyl-4-methyl-benzotriazol-5-yl)propanoic acid;
(3R)-3-[(4R)-2-benzoyl-4-methyl-3,4-dihydro-1H-isoquinolin-7-yl]-3-(1-ethyl-4-methyl-benzotriazol-5-yl)propanoic acid;
(3S)-3-(2-benzoyl-5-methyl-3,4-dihydro-1H-isoquinolin-7-yl)-3-(1-ethyl-4-methyl-benzotriazol-5-yl)propanoic acid; N-ethylethanamine
(3R)-3-(2-benzoyl-5-methyl-3,4-dihydro-1H-isoquinolin-7-yl)-3-(1-ethyl-4-methyl-benzotriazol-5-yl)propanoic acid; N-ethylethanamine
3-(2-benzoyl-5-nitro-3,4-dihydro-1H-isoquinolin-7-yl)-3-(1-ethyl-4-methyl-benzotriazol-5-yl)propanoic acid;
(3S)-3-(1-ethyl-4-methyl-benzotriazol-5-yl)-3-[5-methyl-2-(2,3,5,6-tetramethylbenzoyl)-3,4-dihydro-1H-isoquinolin-7-yl]propanoic acid;
(3R)-3-(1-ethyl-4-methyl-benzotriazol-5-yl)-3-[5-methyl-2-(2,3,5,6-tetramethylbenzoyl)-3,4-dihydro-1H-isoquinolin-7-yl]propanoic acid;
(3S)-3-(1-ethyl-4-methyl-benzotriazol-5-yl)-3-[2-(naphthalene-2-carbonyl)-3,4-dihydro-1H-isoquinolin-7-yl]propanoic acid;
(3R)-3-(1-ethyl-4-methyl-benzotriazol-5-yl)-3-[2-(naphthalene-2-carbonyl)-3,4-dihydro-1H-isoquinolin-7-yl]propanoic acid;
3-(2-benzoyl-3-methyl-3,4-dihydro-1H-isoquinolin-7-yl)-3-(1-ethyl-4-methyl-benzotriazol-5-yl)propanoic acid;
3-(2-benzoyl-3-methyl-1,2,3,4-tetrahydroisoquinolin-7-yl)-3-(1-ethyl-4-methyl-1H-benzo[d][1,2,3]triazol-5-yl)propanoic acid;
3-(1-ethyl-4-methyl-benzotriazol-5-yl)-3-[2-(isoquinoline-3-carbonyl)-3,4-dihydro-1H-isoquinolin-7-yl]propanoic acid;
3-(1-ethyl-4-methyl-benzotriazol-5-yl)-3-[2-(2-methylthiazole-4-carbonyl)-3,4-dihydro-1H-isoquinolin-7-yl]propanoic acid;
3-[2-(1H-benzimidazole-2-carbonyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-(6-methylpyridine-2-carbonyl)-3,4-dihydro-1H-isoquinolin-7-yl]propanoic acid;
3-(1-ethyl-4-methyl-benzotriazol-5-yl)-3-[2-(2-methylpyridine-3-carbonyl)-3,4-dihydro-1H-isoquinolin-7-yl]propanoic acid;
3-[2-(2,5-dimethylbenzoyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-[2-(trifluoromethyl)benzoyl]-3,4-dihydro-1H-isoquinolin-7-yl]propanoic acid;
3-(1-ethyl-4-methyl-benzotriazol-5-yl)-3-[2-(2-fluoro-5-methyl-benzoyl)-3,4-dihydro-1H-isoquinolin-7-yl]propanoic acid;
3-(1-ethyl-4-methyl-benzotriazol-5-yl)-3-[2-(1H-indole-5-carbonyl)-3,4-dihydro-1H-isoquinolin-7-yl]propanoic acid; and
3-[2-(3-cyanobenzoyl)-3,4-dihydro-1H-isoquinolin-7-yl]-3-(1-ethyl-4-methyl-benzotriazol-5-yl)propanoic acid,
   and a pharmaceutically acceptable salt thereof.

As used herein, the term “alkyl” refers to a fully saturated branched or unbranched hydrocarbon moiety. Unless otherwise specified, the alkyl comprises 1 to 12 carbon atoms, preferably 1 to 8 carbon atoms, more preferably 1 to 6 carbon atoms or most preferably 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl and n-hexyl.

As used herein, the term “alkenyl” refers to an unsaturated hydrocarbon group which may be linear or branched and has at least one carbon-carbon double bond. Alkenyl groups with 2-12 carbon atoms or 2-6 carbon atoms are preferred. The alkenyl group may contain 1, 2 or 3 carbon-carbon double bonds, or more. Preferably, alkenyl groups contain one or two double bonds, most preferably one double bond. Examples of alkenyl groups include ethenyl, n-propenyl, isopropenyl, n-but-2-enyl, n-hex-3-enyl and the like.

As used herein, the term “alkynyl” refers to an unsaturated hydrocarbon group which is linear or branched and has at least one carbon-carbon triple bond. Alkynyl groups with 2-12 carbon atoms or 2-6 carbon atoms can be preferred. The alkynyl group may contain 1, 2 or 3 carbon-carbon triple bonds, or more. Preferably, alkynyl groups contain one or two triple bonds, most preferably one triple bond. Examples of alkynyl groups include ethynyl, n-propynyl, n-but-2-ynyl, n-hex-3-ynyl and the like.

As used herein, the term “acyl” refers to a monovalent group with a carbon atom of a carbonyl group as the point of attachment, further having a linear or branched, cyclo, cyclic or acyclic structure, further having no additional atoms that are not carbon or hydrogen, beyond the oxygen atom of the carbonyl group. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, —COC₆H₃(CH₃)₂ and —C(O)CH₂C₆H₅, are non-limiting examples of acyl groups. The term “acyl” therefore encompasses, but is not limited to, groups sometimes referred to as “alkyl carbonyl” and “aryl carbonyl” groups.

As used herein, the term “alkoxy” refers to the group —OR, in which R is a C₁₋₁₂alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopropyl, —O-cyclobutyl, —O-cyclopentyl and —O-cyclohexyl.

The number of carbon atoms in a group is specified herein by the prefix “Cₓ₋ₓₓ”, wherein x and xx are integers. For example, “C₁₋₄alkyl” is an alkyl group which has from 1 to 4 carbon atoms.

As used herein, the term “halogen” or “halo” may be fluoro, chloro, bromo or iodo.

As used herein, the term “heterocyclyl” refers to a saturated or unsaturated, monocyclic or bicyclic (e.g., bridged, fused or spiro) ring system which has from 3- to 12-ring members, or in particular 3- to 6-ring members or 5- to 7-ring members, at least one of which is a heteroatom, and up to 4 (e.g., 1, 2, 3 or 4) of which may be heteroatoms, wherein the heteroatoms are independently selected from O, S and N, and wherein C can be oxidized (e.g., C(O)), N can be oxidized (e.g., N(O)) or quaternized, and S can be optionally oxidized to sulfoxide and sulfone. Unsaturated heterocyclic rings include heteroaryl rings and heterocyclic rings that is not aromatic (i.e., “non-aromatic heterocycles”). As used herein, the term “heteroaryl” refers to an aromatic 5 to 12 membered monocyclic or bicyclic ring system, having 1 to 4 heteroatoms independently selected from O, S and N, and wherein N can be oxidized (e.g., N(O)) or quaternized, and S can be optionally oxidized to sulfoxide and sulfone. A non-aromatic heterocyclyl is a 3- to 7-membered saturated monocyclic or a 3- to 6-membered saturated monocyclic or a 5- to 7-membered saturated monocyclic ring. A non-aromatic heterocyclyl is a 3- to 7-membered unsaturated monocyclic or a 3- to 6-membered unsaturated monocyclic or a 5- to 7-membered unsaturated monocyclic ring. In another embodiment, a heterocyclyl is a 6 or-7-membered bicyclic ring. The heterocyclyl group can be attached at a heteroatom or a carbon atom. Examples of non-aromatic heterocyclyls include aziridinyl, azetidinyl, oxetanyl, thietanyl, pyrrolidinyl, tetrahydrofuranyl, thiolanyl, imidazolidinyl, pyrazolidinyl, isoxazolidinyl, isothiazolidinyl, piperidinyl, tetrahydropyranyl, thianyl, piperazinyl, morpholinyl, thiomorpholinyl, dioxanyl, dithianyl, azepanyl, oxepanyl, thiepanyl, dihydrofuranyl, imidazolinyl, dihydropyranyl, dihydrodioxinyl, hydantoinyl, pyrrolidinonyl, tetrahydrothiopyranyl, tetrahydropyridinyl, and thiopyranyl, and examples of heteroaryls including pyrrolyl, furanyl, imidazolyl, pyrazolyl, oxazolyl, isoxazolyl, thiazolyl, isothiazolyl, oxadiazolyl, thiadiazolyl, triazolyl, tetrazolyl, pyridyl, pyranyl, pyrazinyl, pyrimidyl, pyridazinyl, oxazinyl, thiazinyl, dioxinyl, triazinyl, tetrazinyl, azepinyl, oxepinyl, thiepinyl, thiazepinyl, 1-oxo-pyridyl, thienyl, valerolactamyl, azaindolyl, benzimidazolyl, benzo[1,4]dioxinyl, benzofuranyl, benzoisoxazolyl, benzoisothiazolyl, benzothiadiazolyl, benzothiazolyl, benzothienyl, benzotriazolyl, benzoxadiazolyl, benzoxazolyl, cyclopentaimidazolyl, cyclopentatriazolyl, imidazo[1,2-a]pyridyl, indazolyl, indolizinyl, indolyl, isoquinolinyl, oxazolopyridinyl, purinyl, pyrazolo[3,4]pyrimidinyl, pyridopyazinyl, pyridopyrimidinyl, pyrrolo[2,3]pyrimidinyl, pyrrolopyrazolyl, pyrroloimidazolyl, pyrrolotriazolyl, quinazolinyl, quinolinyl, thiazolopyridinyl, and the like. Examples of bicyclic nonaromatic heterocyclic ring systems include benzo[1,3]dioxolyl, tetrahydroindolyl, and 2-azaspiro[3.3]heptanyl.

As used herein, the term “carbocyclyl” refers to saturated, partially unsaturated, or aromatic monocyclic or bicyclic hydrocarbon groups of 3-12 carbon atoms, 3-6 carbon atoms or 5-7 carbon atoms. The term “carbocyclyl” encompasses cycloalkyl groups and aromatic groups. The term “cycloalkyl” refers to completely saturated monocyclic or bicyclic (e.g., bridged, fused or spiro) hydrocarbon groups of 3-12 carbon atoms, 3-6 carbon atoms or 5-7 carbon atoms. “Aromatic group or “aryl” refers to an aromatic 6-12 membered monocyclic or bicyclic ring system. Exemplary monocyclic carbocyclyl groups include, but are not limited to, cyclopropyl, cyclobutyl, cyclopentyl, cyclohexyl, cycloheptyl, cyclopropenyl, cyclobutenyl, cyclopenentyl, cyclohexenyl, cycloheptenyl, cyclobutadienyl, cyclopentadienyl, cyclohexadienyl, cycloheptadienyl, phenyl and cycloheptatrienyl.

The term “bridged ring system,” as used herein, is a ring system that has a carbocyclyl or heterocyclyl ring wherein two non-adjacent atoms of the ring are connected (bridged) by one or more (preferably from one to three) atoms selected from C, N, O or S. A bridged ring system may have 6-12 ring members. Exemplary bridged carbocyclyl groups include decahydro-2,7-methanonaphthyl, bicyclo[2.2.1]heptyl, bicyclo[2.1.1]hexyl, bicyclo[2.2.1]heptenyl, tricyclo[2.2.1.0^{2,6}]heptanyl, 6,6-dimethylbicyclo[3.1.1]heptyl, and 2,6,6-trimethylbicyclo[3.1.1]heptyl. Exemplary bridged heterocyclyl groups include heterobicyclo[2.2.1]heptenyl and heterobicyclo[3.2.1]octenyl. The specific examples of the bridged heterocyclyl groups include (1S,4R)-2-azabicyclo[2.2.1]hept-5-enyl, (4S)-2-azabicyclo[2.2.1]hept-5-enyl, and (1R,5S)-8-azabicyclo[3.2.1]oct-2-enyl.

The term “fused ring system,” as used herein, is a ring system that has a carbocyclyl or heterocyclyl ring wherein two adjacent atoms of the ring are connected (bridged) by one or more (preferably from one to three) atoms selected from C, N, O or S. A fused ring system may have from 4-10 ring members.

The term “spiro ring system,” as used herein, is a ring system that has two rings each of which are independently selected from a carbocyclyl or a heterocyclyl, wherein the two ring structures having one ring atom in common. Spiro ring systems have from 5 to 7 ring members. Exemplary spiro ring carbocyclyl groups include spiro[2.2]pentanyl and spiro[3.3]heptanyl.

Pharmaceutical acceptable salts of the compounds disclosed herein are also included in the invention. In cases where a compound provided herein is sufficiently basic or acidic to form stable nontoxic acid; or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate or α-glycerophosphate. Inorganic salts may also be formed, including hydrochloride, sulfate, nitrate, bicarbonate and carbonate salts.

Pharmaceutically acceptable salts may be obtained using standard procedures well known in the art, for example by reacting a sufficiently basic compound such as an amine with a suitable acid; affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases can include, but are not limited to, sodium, potassium, lithium, ammonium, calcium and magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, heterocycloalkyl and the like. Also included are amines where the two or three substituents, together with the amino nitrogen, form a heterocycloalkyl and heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine and the like. Other carboxylic acid; derivatives can be useful, for example, carboxylic acid; amides, including carboxamides, lower alkyl carboxamides or dialkyl carboxamides and the like.

The disclosed compounds, or pharmaceutically acceptable salts thereof, can contain one or more asymmetric centers in the molecule. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis or chromatographic separation using a chiral stationary phase). The disclosed compounds may exist in tautomeric forms and mixtures and separate individual tautomers are contemplated. In addition, some compounds may exhibit polymorphism.

When a particular steroisomer (e.g., enantiomer, diasteromer, etc.) of a compound used in the disclosed methods is depicted by name or structure, the stereochemical purity of the compounds is at least 60%, 70%, 80%, 90%, 95%, 97%, 99%, 99.5% or 99.9%. “Stererochemical purity” means the weight percent of the desired stereoisomer relative to the combined weight of all stereoisomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

In one embodiment, any position occupied by hydrogen is meant to include enrichment by deuterium above the natural abundance of deuterium as well. For example, one or more hydrogen atoms are replaced with deuterium at an abundance that is at least 3340 times greater than the natural abundance of deuterium, which is 0.015% (i.e., at least 50.1% incorporation of deuterium), at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In one embodiment, hydrogen is present at all positions at its natural abundance. The compounds or pharmaceutically acceptable salts thereof as described herein, may exist in tautomeric forms and mixtures and separate individual tautomers are contemplated.

Another embodiment is a pharmaceutical composition comprising at least one compound described herein, or a pharmaceutically acceptable salt thereof, and at least one pharmaceutically acceptable carrier.

The compounds provided herein can be useful to activate the NRF2 pathway in a cell. In one embodiment, the method comprises contacting a cell with an effective amount of a compound provided herein, or a pharmaceutically acceptable salt thereof. In one embodiment, the cell is contacted in vitro or in vivo. In one embodiment, contacting the cell includes administering the compound to a subject.

One embodiment of the invention includes a method for activating Nrf2 in a subject in need thereof, the method comprising administering to the subject a therapeutically effective amount of a compound described herein, or a pharmaceutically acceptable salt thereof, thereby activating Nrf2 in the subject.

One embodiment of the invention includes a method for inhibiting a KEAP1 protein in a cell, the method comprising contacting a cell with an effective amount of a compound provided herein, or a pharmaceutically acceptable salt thereof, thereby inhibiting a KEAP1 protein in the cell.

One embodiment of the invention includes a method for increasing a cell's ability to resist a stress, the method comprising administering to the subject a therapeutically effective amount of a compound described herein, or a pharmaceutically acceptable salt thereof, thereby increasing the cell's ability to resist the stress. The stress is selected from the group consisting of heat shock, oxidative stress, osmotic stress, DNA damage, inadequate salt level, inadequate nitrogen level and inadequate nutrient level.

One embodiment of the invention includes a method for mimicking the effect of nutrient restriction on the cell, the method comprising administering to the subject a therapeutically effective amount of a compound described herein, or a pharmaceutically acceptable salt thereof, thereby mimicking the effect of the nutrient restriction on the cell.

One embodiment of the invention includes a method for promoting survival of a eukaryotic cell (e.g., a mammalian cell) or increasing the lifespan of the cell, the method comprising administering to the subject a therapeutically effective amount of a compound provided herein, or a pharmaceutically acceptable salt thereof, thereby promoting survival of the eukaryotic cell or increasing the lifespan of the cell.

One embodiment of the invention includes a method for treating a disease associated with cell death in a subject, the method comprising administering to the subject a therapeutically effective amount of a compound described herein, or a pharmaceutically acceptable salt thereof.

One embodiment of the invention includes a method for treating a disease caused by oxidative stress in a subject, the method comprising administering to the subject a therapeutically effective amount of a compound provided herein, or a pharmaceutically acceptable salt thereof.

One embodiment of the invention includes a method for treating a disorder in a subject, wherein the disorder is selected from the group consisting of a neurodegenerative disease, inflammation/an inflammatory disease, an autoimmune disease, an ischemic fibrotic disease, a cancer, premature aging, a cardiovascular disease, a liver disease, a hemoglobinopathy, thalassemia (e.g. beta-thalassemia) and a metabolic disorder, the method comprising administering to the subject a therapeutically effective amount of a compound provided herein, or a pharmaceutically acceptable salt thereof. Hemoglobinopathy includes sickle cell disease (SCD). In one embodiment, the disorder is sickle cell disease or thalassemia (e.g. beta-thalassemia). More specifically, the disorder is sickle cell disease.

The neurodegenerative disease can be selected from the group consisting of Alzheimer's disease (AD), Parkinson's disease (PD), Huntington disease (HD) and other CAG-triplet repeat (or polyglutamine) diseases, amyotrophic lateral sclerosis (ALS; Lou Gehrig's disease), diffuse Lewy body disease, chorea-acanthocytosis, primary lateral sclerosis, multiple sclerosis (MS), frontotemporal dementia, Friedreich's ataxia and epilepsy (repression of microglia activation). More preferably, the neurodegenerative disease is Parkinson's disease or amyotrophic lateral sclerosis.

The inflammatory disease can be selected from the group consisting of chronic cholecystitis, aortic valve stenosis, restenosis, a skin disease, a pulmonary diseases and a disease of the airway, inflammatory uveitis, atherosclerosis, arthritis, conjunctivitis, pancreatitis, a chronic kidney disease (CDK), an inflammatory condition associated with diabetes,an ischemia, a transplant rejection, a CD14 mediated sepsis, a non-CD14 mediated sepsis, Behcet's syndrome, ankylosing spondylitis, sarcoidosis and gout. In some embodiments, the skin disease is selected from the group consisting of rash, contact dermatitis and atopic dermatitis. In one embodiment, the pulmonary disease and disease of the airway is selected from the group consisting of Adult Respiratory Disease Syndrome (ARDS), Chronic Obstructive Pulmonary Disease (COPD), pulmonary fibrosis, an interstitial lung disease, asthma, chronic cough, allergic rhinitis, bronchiectasis and bronchitis. In one embodiment, the inflammatory condition associated with diabetes is selected from a diabetic retinopathy, a diabetic cardiomyopathy and a diabetes-induced aortic damage.

The autoimmune disease is selected from the group consisting of psoriasis, inflammatory bowel disease, rheumatoid arthritis, systemic lupus erythematosus, multiple sclerosis, type 1 diabetes, systemic sclerosis and Sjogren's syndrome. In one embodiment, the inflammatory bowel disease is Crohn's disease or ulcerative colitis. In one embodiment, the autoimmune disease is type 1 diabetes. Alternatively, the autoimmune disease is multiple sclerosis.

The ischemic fibrotic disease is selected from the group consisting of stroke, acute lung injury, acute kidney injury, ischemic cardiac injury, acute liver injury and ischemic skeletal muscle injury.

The cancer is selected from the group consisting of prostate cancer, bladder cancer, ovarian cancer, breast cancer (e.g., breast cancer with mutated BRCA1), head and neck cancer, chronic lymphocytic leukemia, thymus cancer, hepatocellular carcinoma, colorectal cancer, colon cancer, skin cancer, pancreatic cancer, leukemia, lung cancer, glioblastoma, cervical cancer, lymphoma, Waldenström's macroglobulinemia and multiple myeloma.

The cardiovascular disease can be selected from the group consisting of pulmonary arterial hypertension, systemic hypertension, coronary artery disease, peripheral artery disease and atherosclerosis.

The liver disease can be selected from the group consisting of non-alcoholic steatohepititis (NASH), alcoholic liver disease, primary biliary cirrhosis and primary sclerosing cholangitis.

The hemoglobinopathy is a condition that involves a mutation in human beta-globin or an expression control sequence thereof, such as sickle cell disease (SCD) or beta-thalassemia. SCD typically arises from a mutation substituting thymine for adenine in the sixth codon of the beta-chain gene of hemoglobin (i.e., GAG to GTG of the HBB gene). This mutation causes glutamate to valine substitution in position 6 of the Hb beta chain. The resulting Hb, referred to as HbS, has the physical properties of forming polymers under conditions of low oxygen tension. SCD is typically an autosomal recessive disorder. Beta-Thalassemias are a group of inherited blood disorders caused by a variety of mutational mechanisms that result in a reduction or absence of synthesis of β-globin and leading to accumulation of aggregates of unpaired, insoluble a-chains that cause ineffective erythropoiesis, accelerated red cell destruction, and severe anemia. Subjects with beta-thalassemia exhibit variable phenotypes ranging from severe anemia to clinically asymptomatic individuals. The genetic mutations present in β thalassemias are diverse, and can be caused by a number of different mutations. The mutations can involve a single base substitution or deletions or inserts within, near or upstream of the β globin gene. For example, mutations occur in the promoter regions preceding the beta-globin genes or cause production of abnormal splice variants. β⁰ is used to indicate a mutation or deletion which results in no functional β globin being produced. β⁺ is used to indicate a mutation in which the quantity or β globin is reduced or in which the β globin produced has a reduced functionality.

Examples of thalassemias include thalassemia minor, thalassemia intermedia, and thalassemia major.

Thalassemia minor refers to thalassemia where only one of beta-globin alleles bears a mutation. Individuals typically suffer from microcytic anemia. Detection usually involves lower than normal MCV value (<80 fL) plus an increase in fraction of Hemoglobin A2 (>3.5%) and a decrease in fraction of Hemoglobin A (<97.5%). Genotypes can be β⁺/β or β⁰/β.

Thalassemia intermedia refers to a thalassemia intermediate between the major and minor forms. Affected individuals can often manage a normal life but may need occasional transfusions, e.g., at times of illness or pregnancy, depending on the severity of their anemia. Genotypes can be β⁺/β⁺ or β⁰/β.

Thalassemia major refers to a thalassemia where both beta-globin alleles have thalassemia mutations. This is a severe microcytic, hypochromic anemia. If left untreated, it causes anemia, splenomegaly, and severe bone deformities and typically leads to death before age 20. Treatment consists of periodic blood transfusion; splenectomy if splenomegaly is present, and treatment of transfusion-caused iron overload. Cure is possible by bone marrow transplantation. Genotypes include β⁺/β⁰ or β⁰/β⁰ or β⁺/β⁺. Mediterranean anemia or Cooley's anemia has a genotype of β⁰/β⁰ so that no hemoglobin A is produced. It is the most severe form of β-thalasemia.

Although carriers of sickle cell trait do not suffer from SCD, individuals with one copy of HbS and one copy of a gene that codes for another abnormal variant of hemoglobin, such as HbC or Hb beta-thalassemia, typically will have a less severe form of sickle cell disease. For example, another specific defect in beta-globin causes another structural variant, hemoglobin C (HbC). Hemoglobin C (abbreviated as Hb C or HbC) is an abnormal hemoglobin in which substitution of a glutamic acid; residue with a lysine residue at the 6th position of the β-globin chain has occurred. A subject that is a double heterozygote for HbS and HbC (HbSC disease) is typically characterized by symptoms of moderate clinical severity.

Another common structural variant of beta-globin is hemoglobin E (HbE). HbE is an abnormal hemoglobin in which substitution of a glutamic acid; residue with a lysine residue at the 26th position of the β-globin chain has occurred. A subject that is a double heterozygote for HbS and HbE has HbS/HbE syndrome, which usually causes a phenotype similar to HbS/b+ thalassemia, discussed below.

A subject that is a double heterozygote for HbS and β⁰ thalassemia (i.e., HbS/β⁰ thalassemia) can suffer symptoms clinically indistinguishable from sickle cell anemia.

A subject that is a double heterozygote for HbS and β⁺ thalassemia (i.e., HbS/β⁺ thalassemia) can have mild-to-moderate severity of clinical symptoms with variability among different ethnicities.

Rare combinations of HbS with other abnormal hemoglobins include HbD Los Angeles, G-Philadelphia, HbO Arab, and others.

Nrf2 upregulates fetal hemoglobin which alleviates some of the symptoms of these disorders. Therefore, in some embodiments, the disclosed compositions are used to treated SCD or thalassemia (e.g. beta-thalassemia), including those that involve a mutation in human beta-globin or an expression control sequence thereof, as described above.

In some embodiments, the disclosed compositions and methods are used to treat a subject with an HbS/β⁰ genotype, an HbS/β⁺ genotype, an HBSC genotype, an HbS/HbE genotype, an HbD Los Angeles genotype, a G-Philadelphia genotype, or an abHbO Arab genotype.

In some embodiments, the compositions disclosed herein are administered to a subject in a therapeutically effective amount to treat one or more symptoms of sickle cell disease, a thalassemia (e.g. beta-thalassemia), or a related disorder. In subjects with sickle cell disease, or a related disorder, physiological changes in RBCs can result in a disease with the following signs: (1) hemolytic anemia; (2) vaso-occlusive crisis; and (3) multiple organ damage from microinfarcts, including heart, skeleton, spleen, and central nervous system. Thalassemia can include symptoms such as anemia, fatigue and weakness, pale skin or jaundice (yellowing of the skin), protruding abdomen with enlarged spleen and liver, dark urine, abnormal facial bones and poor growth, and poor appetite.

Retinopathy due to SCD can also be treated by administering a therapeutically effective amount of a compound according to any one of described herein. Sickle retinopathy occurs when the retinal blood vessels get occluded by sickle red blood cells and the retina becomes ischemic, angiogenic factors are made in retina. In sickle cell disease, this occurs mostly in the peripheral retina, which does not obscure vision at first. Eventually, the entire peripheral retina of the sickle cell patient becomes occluded and many neovascular formations occur. Administration of a compound according to any one of described herein can reduce or inhibit the formation of occlusions in the peripheral retina of a sickle cell patient.

As used herein, the term “subject” and “patient” may be used interchangeably, and means a mammal in need of treatment, e.g., companion animals (e.g., dogs, cats and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or ‘treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; and delaying, inhibiting or decreasing the likelihood of the progression of the disease, disorder or syndrome.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, orally, topically, enterally (e.g. orally), parenterally, transdermally, transmucosally, via inhalation, intracisternally, epidurally, intravaginally, intravenously, intramuscularly, subcutaneously, intradermally and intravitreally to the mammal. Administering a compound described herein, or a pharmaceutically acceptable salt thereof, to a mammal also includes administering topically, enterally (e.g. orally), parenterally, transdermally, transmucosally, via inhalation, intracisternally, epidurally, intravaginally, intravenously, intramuscularly, subcutaneously, intradermally and intravitreally to a mammal a compound that metabolizes within or on a surface of the body of the mammal to a compound described herein, or a pharmaceutically acceptable salt thereof.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups or wafers and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that a therapeutically effective dosage level will be obtained.

The tablets, troches, pills, capsules and the like can include the following: binders such as gum tragacanth, acacia, corn starch and gelatin; excipients such as dicalcium phosphate; a disintegrating agent such as corn starch, potato starch, alginic acid; and the like; a lubricant such as magnesium stearate; a sweetening agent such as sucrose, fructose, lactose and aspartame; and a flavoring agent.

The active compound may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions and sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols, glycols and water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity and in vivo activity in animal models. Methods for the extrapolation of effective dosages in mice and other animals, to humans are known to the art; for example, see U.S. Pat. No. 4,938,949, which is incorporated by reference in its entirety.

“A therapeutically effective amount” and “an effective amount” are interchangeable and refer to an amount that, when administered to a subject, achieves a desired effect for treating a disease treatable with a compound or pharmaceutically acceptable salt thereof as described herein. The therapeutically effective amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 μg to about 100 mg/kg of body weight per day.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

### EXAMPLE

The terms “Ent1” and Ent2” do not infer structural assignment as to one enantiomer or the other. The absolute configuration of final compounds was only determined in certain instances as described below:

### Key:

A: Absolute configuration determined by x-ray crystallography and/or circular dichroism
B: Absolute configuration assigned by comparison to a class A compound or derived from a common intermediate in the synthesis of a class A compound
C: Absolute configuration assigned based on literature precedent
D: Absolute configuration unknown

| | Stereochemical |
|---|---|
| Compound | Determination |
| 12-Ent1 | D |
| 12-Ent2 | D |
| 31-Ent1 | D |
| 31-Ent2 | D |
| 41-Ent1 | D |
| 41-Ent2 | D |
| 47-Ent1 | B |
| 47-Ent2 | B |
| 48-Ent1 | D |
| 48-Ent2 | D |
| 49-Ent1 | A |
| 49-Ent2 | B |
| 52-Ent1 | D |
| 52-Ent2 | D |
| 53 | B |
| 54 | B |
| 55-Isomer 1 | D |
| 55-Isomer 2 | D |
| 57-Ent1 | D |
| 57-Ent2 | D |
| 64-Ent1 | D |
| 64-Ent2 | D |
| 65-Ent1 | D |
| 65-Ent2 | D |
| 66-Ent1 | A |
| 66-Ent2 | B |
| 68 | D |
| 69 | D |
| 70-Ent1 | D |
| 70-Ent2 | D |
| 72-Isomer 3 | D |
| 92 | D |
| 93 | D |
| 94 | D |
| 95 | D |
| 100 | D |
| 101 | D |
| 102 | B |
| 103 | B |
| 104 | B |
| 105 | B |
| 107 | B |
| 108 | B |
| 109 | B |
| 110 | B |
| 111 | B |
| 114 | B |
| 115 | D |
| 116 | D |
| 117 | D |
| 118 | D |
| 119 | D |
| 120 | D |
| 121 | D |
| 122 | D |
| 123 | B |
| 124 | B |
| 125 | B |
| 126 | B |
| 127 | B |
| 128 | B |
| 129 | B |
| 130 | B |
| 131 | B |
| 132 | B |
| 133 | B |
| 134 | D |
| 135 | D |
| 139 | D |
| 140 | D |
| 142 | D |
| 144 | D |
| 145 | D |
| 149 | D |
| 151 | B |
| 152 | B |
| 153 | D |
| 154 | D |
| 155 | D |
| 156 | D |
| 157 | D |
| 158 | D |
| 159 | D |
| 160 | D |
| 161 | D |
| 162 | D |

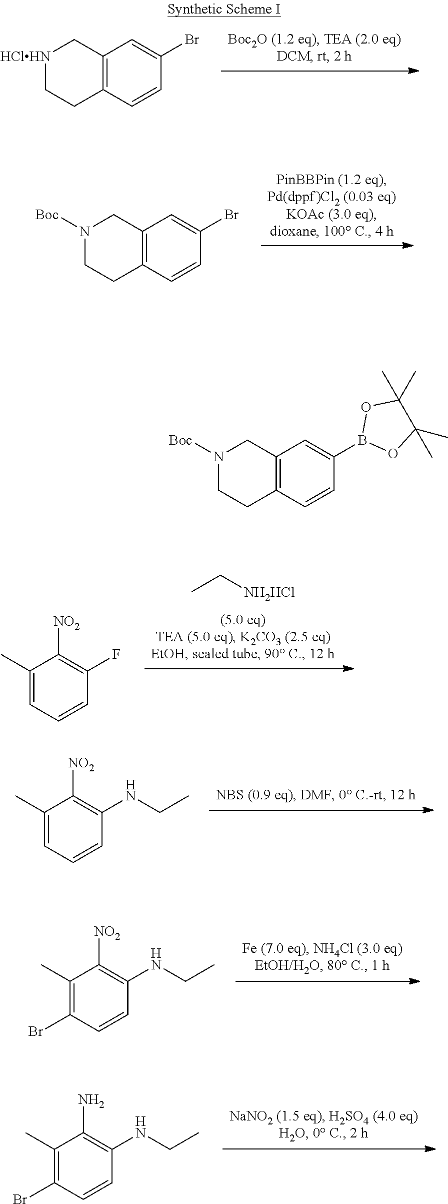

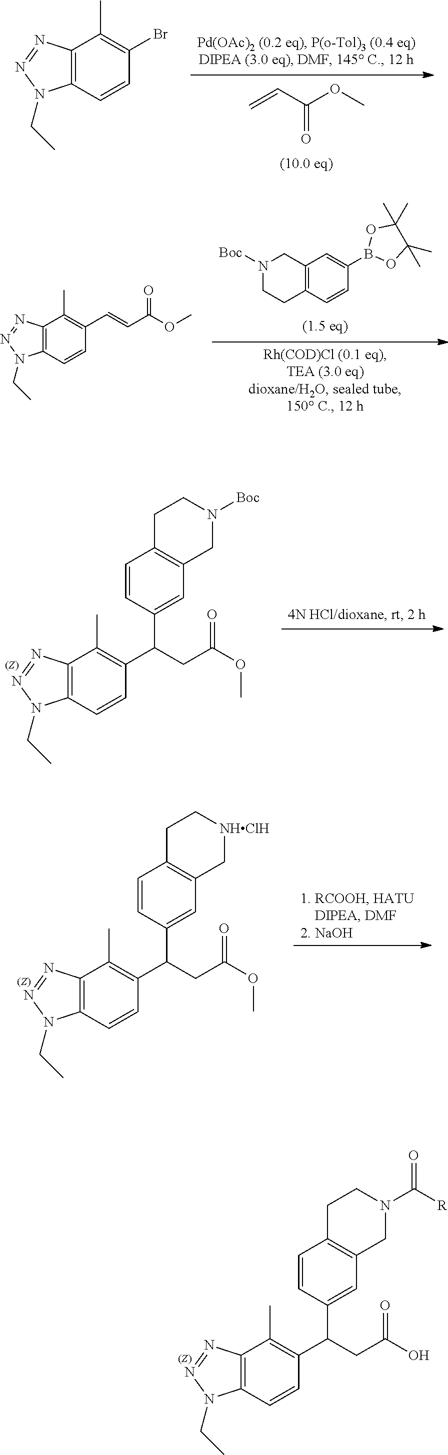

### Example 1

### 3-(1-ethyl-4-methyl-1H-benzo[d][1,2,3]triazol-5-yl)-3-(2-(4-methylbenzoyl)-1,2,3,4-tetrahydroisoquinolin-7-yl)propanoic acid;

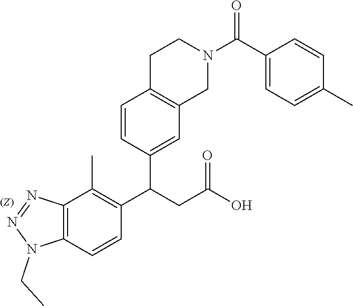 [SMILES: CCn1nnc2c(C)c(C(CC(=O)O)c3ccc4c(c3)CN(C(=O)c3ccc(C)cc3)CC4)ccc21]

### 1. Synthesis of tert-butyl 7-bromo-3,4-dihydroisoquinoline-2(1H)-carboxylate

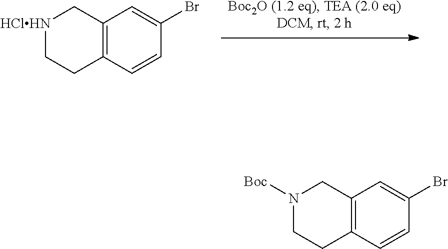

To a mixture of 7-bromo-1,2,3,4-tetrahydroisoquinoline HCl salt (37 g, 150 mmol) and TEA (30 g, 300 mmol) in DCM (400 mL), Boc₂O (41 g, 180 mmol) was added. The mixture was stirred at rt for 2 h, then diluted with DCM (500 mL). The organic layer was washed with water (300 mL), brine (300 mL), dried over sodium sulfate and concentrated under reduced pressure. The residue was purified by silica gel column (PE:EA=10:1) to give tert-butyl 7-bromo-3,4-dihydroisoquinoline-2(1H)-carboxylate (42 g, yield: 90%) as a white solid. ESI-MS (M−56+H)⁺: 256.1. ¹H NMR (400 MHz, CDCl₃) δ: 7.28-7.25 (m, 2H), 7.01 (d, J=8.0 Hz, 1H), 4.53 (s, 2H), 3.63 (t, J=5.2 Hz, 2H), 2.84 (t, J=5.2 Hz, 2H), 1.48 (s, 9H).

### 2. Synthesis of tert-butyl 7-(4,4,5,5-tetramethyl-1,3,2-dioxaborolan-2-yl)-3,4-dihydroisoquinoline-2(1H)-carboxylate

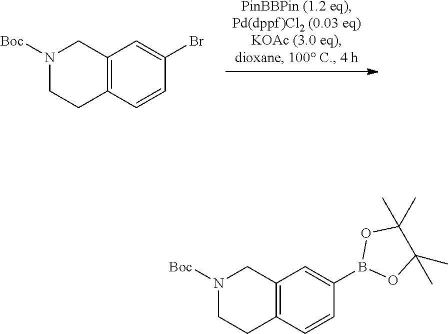 [SMILES: "PinBBPin (1.2 eq),Pd(dppf)Cl2 (0.03 eq)KOAc (3.0 eq),dioxane, 100° C., 4 h" ; CC(C)(C)OC(=O)N1CCc2ccc(Br)cc2C1 ; CC(C)(C)OC(=O)N1CCc2ccc(B3OC(C)(C)C(C)(C)O3)cc2C1]

To a mixture of tert-butyl 7-bromo-3,4-dihydroisoquinoline-2(1H)-carboxylate (42 g, 135 mmol) and PinB-BPin (36 g, 141 mmol) in dioxane (300 mL), KOAc (40 g, 405 mmol) was added. Then, Pd(dppf)Cl₂.DCM (3 g, 4 mmol) was added quickly under N₂ atmosphere. The mixture was stirred at 100° C. for 4 h under N₂ atmosphere. After cooling down, the salts were filtered out, the resulting filtrate was concentrated and purified by silica gel column (PE:EA=20:1) to give tert-butyl 7-(4,4,5,5-tetramethyl-1,3,2-dioxaborolan-2-yl)-3,4-dihydroisoquinoline-2(1H)-carboxylate (45 g, yield: 92%) as a white solid. ESI-MS (M+Na)⁺: 382.1. ¹H NMR (400 MHz, CDCl₃) δ: 7.59 (d, J=7.6 Hz, 1H), 7.56 (s, 1H), 7.14 (d, J=7.2 Hz, 1H), 4.58 (s, 2H), 3.62 (t, J=5.2 Hz, 2H), 2.77 (t, J=5.2 Hz, 2H), 1.48 (s, 9H), 1.34 (s, 12H).

### 3. Synthesis of N-ethyl-3-methyl-2-nitroaniline

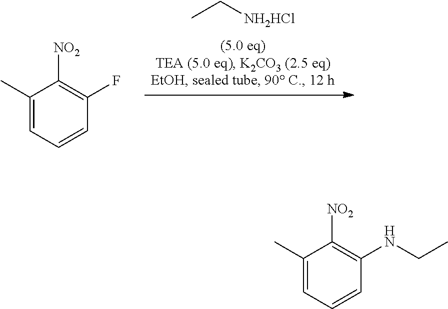

To a mixture of 1-fluoro-3-methyl-2-nitrobenzene (9.3 g, 60 mmol) and ethylamine HCl salt (24.3 g, 300 mmol) in EtOH (250 mL), TEA (15 g, 150 mmol) and K₂CO₃ (20.7 g, 150 mmol) were added. The mixture was sealed and stirred at 90° C. for 12 h. After cooling down, the reaction mixture was diluted with EA (300 mL). The organic layer was washed with water (100 mL), brine (100 mL), dried over sodium sulfate and concentrated under reduced pressure to give N-ethyl-3-methyl-2-nitroaniline (10.5 g, yield: 97%) as a yellow oil. ESI-MS (M+H)⁺: 181.1. ¹H NMR (400 MHz, CDCl₃) δ: 7.21 (t, J=8.0 Hz, 1H), 6.65 (d, J=8.4 Hz, 1H), 6.45 (brs, 1H), 7.51 (d, J=7.2 Hz, 1H), 3.27-3.20 (m, 2H), 2.46 (s, 3H), 1.31 (t, J=7.2 Hz, 3H).

### 4. Synthesis of 4-bromo-N-ethyl-3-methyl-2-nitroaniline

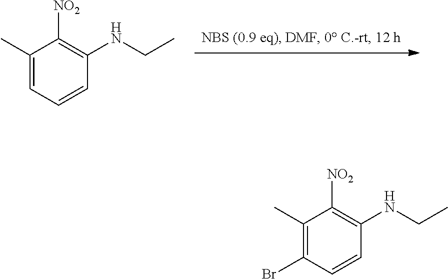

To a mixture of N-ethyl-3-methyl-2-nitroaniline (20 g, 110 mmol) in DMF (125 mL), NBS (17.5 g, 100 mmol) in DMF (125 mL) was added dropwise at 0° C., and then the mixture was warmed to rt. After stirred at rt for 12 h, the reaction mixture was diluted with EA (1000 mL). The organic layer was washed with brine (200 mL×5), dried over sodium sulfate and concentrated under reduced pressure. The residue was recrystallized from PE to give 4-bromo-N-ethyl-3-methyl-2-nitroaniline (21.2 g, yield: 75%) as a yellow solid. ESI-MS (M+H)⁺: 259.0. ¹H NMR (400 MHz, CDCl₃) δ: 7.46 (d, J=9.2 Hz, 1H), 6.55 (d, J=9.2 Hz, 1H), 5.60 (brs, 1H), 3.23-3.16 (m, 2H), 2.43 (s, 3H), 1.28 (t, J=7.2 Hz, 3H).

### 5. Synthesis of 4-bromo-N1-ethyl-3-methylbenzene-1,2-diamine

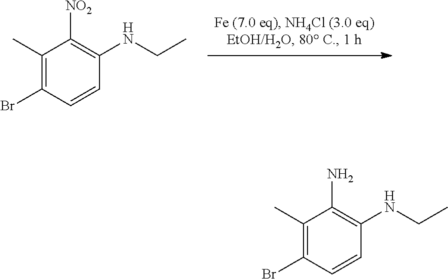

To a mixture of 4-bromo-N-ethyl-3-methyl-2-nitroaniline (28 g, 108 mmol) in EtOH (300 mL)/H₂O (100 mL), Fe (4.2 g, 760 mmol) and NH₄Cl (17 g, 324 mmol) were added. The mixture was stirred at 80° C. for 1 h and then concentrated under reduced pressure. The residue was purified by silica gel column (PE:EA=4:1) to give 4-bromo-N1-ethyl-3-methylbenzene-1,2-diamine (20 g, yield: 81%) as a yellow solid. ESI-MS (M+H)⁺: 229.1. ¹H NMR (400 MHz, CDCl₃) δ: 7.01 (d, J=8.8 Hz, 1H), 6.44 (d, J=8.4 Hz, 1H), 3.44 (br, 2H), 3.18-3.02 (m, 2H), 2.31 (s, 3H), 1.29 (t, J=7.2 Hz, 3H).

### 6. Synthesis of 5-bromo-1-ethyl-4-methyl-1H-benzo[d][1,2,3]triazole

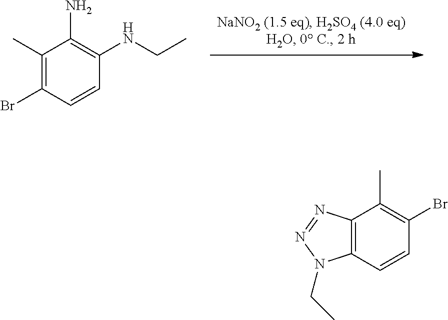

To a mixture of 4-bromo-N1-ethyl-3-methylbenzene-1,2-diamine (20 g, 87 mmol) in conc. H₂SO₄ (34 g), a solution of NaNO₂ (9 g, 130 mmol) in H₂O (350 mL) was added dropwise at 0° C. The mixture was stirred at 0° C. for 2 h and then diluted with H₂O (300 mL). The precipitate was collected by filtration. The crude residue was purified by silica gel column (PE:EA=4:1) to give 5-bromo-1-ethyl-4-methyl-1H-benzo[d][1,2,3]triazole (13.2 g, yield: 60%) as a brown solid. ESI-MS (M+H)⁺: 240.1. ¹H NMR (400 MHz, CDCl₃) δ: 7.59 (d, J=8.8 Hz, 1H), 7.23 (d, J=8.4 Hz, 1H), 4.66 (q, J=7.2 Hz, 2H), 2.83 (s, 3H), 1.61 (t, J=7.2 Hz, 3H).

### 7. Synthesis of methyl (E)-3-(1-ethyl-4-methyl-1H-benzo[d][1,2,3]triazol-5-yl)acrylate

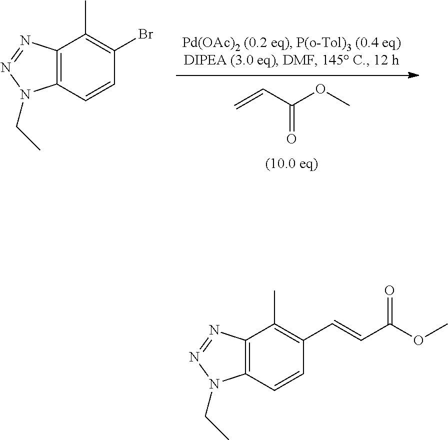

To a mixture of 5-bromo-1-ethyl-4-methyl-1H-benzo[d][1,2,3]triazole (13.6 g, 57 mmol) and methyl acrylate (49 g, 570 mmol) in DMF (300 mL), DIPEA (22 g, 171 mmol) was added. Then, Pd(OAc)₂ (2.5 g, 11 mmol) and P(o-tol)₃ (6.9 g, 22 mmol) were added quickly under N₂ atmosphere. The mixture was stirred at 145° C. for 12 h under N₂ atmosphere. After cooling down, the solvent was removed under reduced pressure. The residue was purified by silica gel column (PE:EA=4:1) to give methyl (E)-3-(1-ethyl-4-methyl-1H-benzo[d][1,2,3]triazol-5-yl)acrylate (11.1 g, yield: 83%) as a yellow solid. ESI-MS (M+Na)⁺: 246.1. ¹H NMR (400 MHz, CDCl₃) δ: 8.16-8.12 (m, 1H), 7.69 (d, J=9.2 Hz, 1H), 7.36 (d, J=8.0 Hz, 1H), 6.43-6.40 (m, 1H), 4.67 (q, J=7.2 Hz, 2H), 3.83 (s, 3H), 2.93 (s, 3H), 1.63 (t, J=7.2 Hz, 3H).

### 8. Synthesis of tert-butyl 7-(1-(1-ethyl-4-methyl-1H-benzo[d][1,2,3]triazol-5-yl)-3-methoxy-3-oxopropyl)-3,4-dihydroisoquinoline-2(1H)-carboxylate

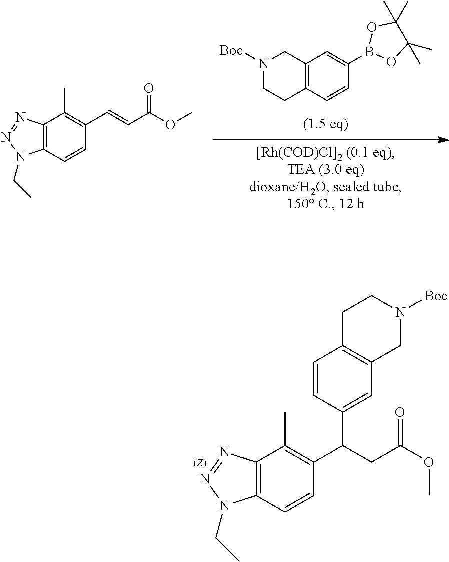

A mixture of methyl (E)-3-(1-ethyl-4-methyl-1H-benzo[d][1,2,3]triazol-5-yl)acrylate (5 g, 20 mmol), tert-butyl 7-(4,4,5,5-tetramethyl-1,3,2-dioxaborolan-2-yl)-3,4-dihydroisoquinoline-2(1H)-carboxylate (10.7 g, 30 mmol) and TEA (6.0 g, 60 mmol) in dioxane/H₂O (50 mL/10 mL) was degassed for 10 min before [Rh(COD)Cl]₂ (980 mg, 2 mmol) was added, and then the mixture was degassed for another 5 min. The mixture was stirred at 150° C. for 12 h in a sealed tube. After cooling down, the solvent was removed under reduced pressure. The residue was purified by silica gel column (PE:EA=2:1) to give tert-butyl 7-(1-(1-ethyl-4-methyl-1H-benzo[d][1,2,3]triazol-5-yl)-3-methoxy-3-oxopropyl)-3,4-dihydroisoquinoline-2(1H)-carboxylate (5 g, yield: 51%) as a yellow oil. ESI-MS (M+H)⁺: 479.2. ¹H NMR (400 MHz, CDCl₃) δ: 7.32-7.31 (m, 2H), 7.04-7.02 (m, 2H), 6.91 (s, 1H), 4.96 (t, J=8.0 Hz, 1H), 4.64 (q, J=7.2 Hz, 2H), 4.48 (s, 2H), 3.63-3.54 (m, 5H), 3.17-3.01 (m, 2H), 2.85 (s, 3H), 2.76 (t, J=4.8 Hz, 2H), 1.59 (t, J=7.2 Hz, 3H), 1.26 (s, 9H).

### 9. Synthesis of methyl 3-(1-ethyl-4-methyl-benzotriazol-5-yl)-3-(1,2,3,4-tetrahydroisoquinolin-7-yl)propanoate

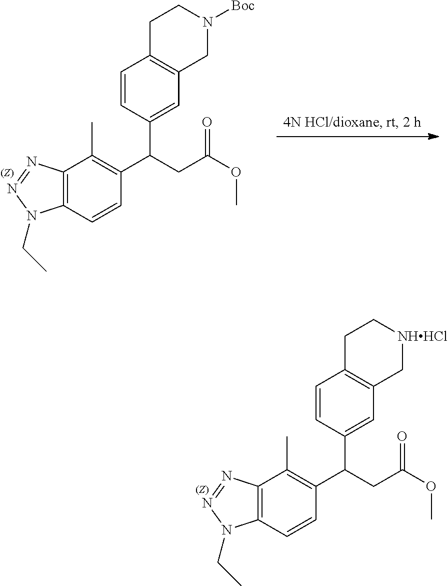

A solution of tert-butyl 7-(1-(1-ethyl-4-methyl-1H-benzo[d][1,2,3]triazol-5-yl)-3-methoxy-3-oxopropyl)-3,4-dihydroisoquinoline-2(1H)-carboxylate (13.8 g, 28 mmol) in HCl/dioxane (4 N, 100 mL) stirred at rt for 2 h. After the half of the solvent was removed under reduced pressure, the mixture was diluted with Et₂O (100 mL). The solvent was decanted and the resulting residue was dissolved in ultrapure H₂O (100 mL) and lyophilized to give methyl 3-(1-ethyl-4-methyl-benzotriazol-5-yl)-3-(1,2,3,4-tetrahydroisoquinolin-7-yl)propanoate (9.6 g, yield: 80%) as a white solid. ESI-MS (M+H)+: 379.2. ¹H NMR (400 MHz, CD₃OD) δ: 7.81-7.69 (m, 2H), 7.27-7.18 (m, 3H), 5.01 (t, J=7.6 Hz, 1H), 4.73 (q, J=7.2 Hz, 2H), 4.31 (s, 2H), 3.57 (s, 3H), 3.48 (t, J=6.4 Hz, 2H), 3.24-3.13 (m, 2H), 3.08 (t, J=6.0 Hz, 2H), 2.79 (s, 3H), 1.60 (t, J=7.2 Hz, 3H).

### 10. The Preparation of 3-(1-ethyl-4-methyl-1H-benzo[d][1,2,3]triazol-5-yl)-3-(2-(4-methylbenzoyl)-1,2,3,4-tetrahydroisoquinolin-7-yl)propanoic acid;

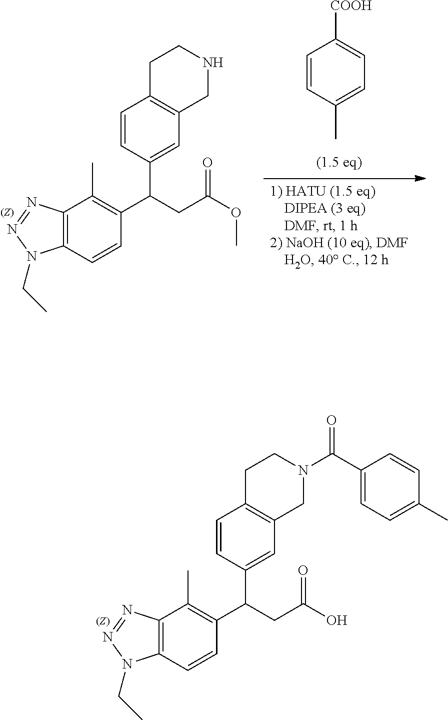

A mixture of 4-methylbenzoic acid; (42 mg, 0.31 mmol), HATU (120 mg, 0.31 mmol), DIPEA (80 mg, 0.62 mmol) in DMF (2 mL) was stirred at rt for 20 min. A solution of methyl 3-(1-ethyl-4-methyl-benzotriazol-5-yl)-3-(1,2,3,4-tetrahydroisoquinolin-7-yl)propanoate (80 mg, 0.21 mmol) in DMF (0.5 mL) was added to the mixture. The reaction was stirred at rt for 1 h. Then a solution of NaOH (84 mg, 2.1 mmol) in H₂O (0.5 mL) was added in the mixture. The reaction was stirred at 40° C. for another 12 h and then acid;ified to pH=1-3 with 6 M HCl, extracted by EA (10 mL×3). The combined organic phase was dried by Na₂SO₄, concentrated. The residue was purified by prep-HPLC (MeCN/water with 0.05% HCOOH as mobile phase) to give 3-(1-ethyl-4-methyl-1H-benzo[d][1,2,3]triazol-5-yl)-3-(2-(4-methylbenzoyl)-1,2,3,4-tetrahydroisoquinolin-7-yl)propanoic acid; (38 mg, yield: 20%) as white solid. ESI-MS (M+H)⁺: 483.2. ¹H NMR (400 MHz, CD₃OD) δ: 7.53-7.49 (m, 2H), 7.33-7.30 (m, 4H), 7.16-6.87 (m, 3H), 4.86-4.79 (m, 1H), 4.71-4.56 (m, 4H), 3.91-3.90 (m, 1H), 3.63-3.60 (m, 1H), 3.34-3.32 (m, 2H), 3.16-3.11 (m, 5H), 2.40 (s, 3H), 1.58 (t, J=7.2 Hz, 3H).

### Example 2

### 3-(1-ethyl-4-methyl-1H-benzo[d][1,2,3]triazol-5-yl)-3-(2-(4-methoxybenzoyl)-1,2,3,4-tetrahydroisoquinolin-7-yl)propanoic acid;

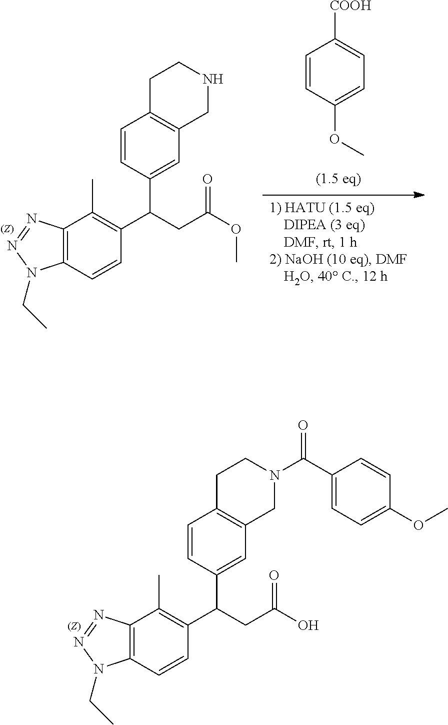

Synthesis of 3-(1-ethyl-4-methyl-1H-benzo[d][1,2,3]triazol-5-yl)-3-(2-(4-methoxybenzoyl)-1,2,3,4-tetrahydroisoquinolin-7-yl)propanoic acid; was similar to that of Example 1. The residue was purified by prep-HPLC (MeCN/water with 0.05% HCOOH as mobile phase) to give 3-(1-ethyl-4-methyl-1H-benzo[d][1,2,3]triazol-5-yl)-3-(2-(4-methoxybenzoyl)-1,2,3,4-tetrahydroisoquinolin-7-yl)propanoic acid; as yellow solid (36 mg, yield: 22%). ESI-MS (M+H)⁺: 499.2. ¹H NMR (400 MHz, CDCl₃) δ: 7.40 (d, J=8.8 Hz, 2H), 7.33 (brs, 3H), 7.06 (s, 2H), 6.91 (d, J=8.8 Hz, 2H), 4.95-4.93 (m, 1H), 4.68-4.61 (m, 4H), 3.83 (s, 3H), 3.72-3.66 (m, 2H), 3.15-3.06 (m, 2H), 2.82-2.80 (m, 5H), 1.60 (t, J=7.2 Hz, 3H).

### Example 3

### 3-(1-ethyl-4-methyl-1H-benzo[d][1,2,3]triazol-5-yl)-3-(2-(4-hydroxybenzoyl)-1,2,3,4-tetrahydroisoquinolin-7-yl)propanoic acid;

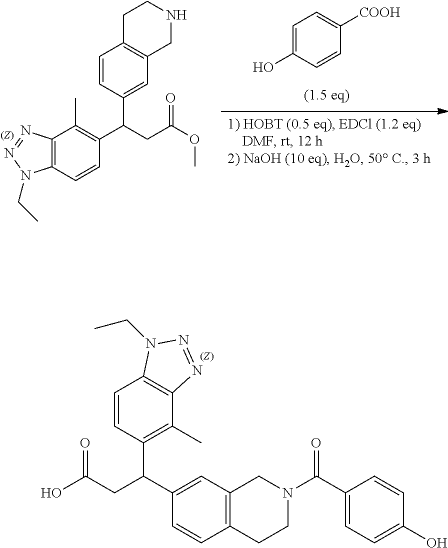

To a mixture of methyl 3-(1-ethyl-4-methyl-1H-benzo[d][1,2,3]triazol-5-yl)-3-(1,2,3,4-tetrahydroisoquinolin-7-yl)propanoate (80 mg, 0.21 mmol) in DMF (5 mL) was added 4-hydroxybenzoic acid; (23 mg, 0.17 mmol), HOBT (14 mg, 0.1 mmol), EDCI (40 mg, 0.21 mmol) and DIPEA (44 mg, 0.34 mmol). The mixture was stirred at rt for 12 h. NaOH (68 mg, 1.7 mmol) and H₂O (5 mL) were added thereto. The mixture was stirred at 50° C. for 3 h. After diluted with H₂O (10 mL), the mixture was acid;ified with 2N HCl to pH=2-3 and extracted with EA (20 mL×3). The combined organic layers were concentrated under reduced pressure. The crude product was purified by prep-HPLC (MeCN/water with 0.05% HCOOH as mobile phase) to give 3-(1-ethyl-4-methyl-1H-benzo[d][1,2,3]triazol-5-yl)-3-(2-(4-hydroxybenzoyl)-1,2,3,4-tetrahydroisoquinolin-7-yl)propanoic acid; as white solid (55 mg, yield: 54%). ESI-MS (M+H)⁺: 485.2. ¹H NMR (400 MHz, CD₃OD) δ: 7.56-7.46 (m, 2H), 7.33 (d, J=8.0 Hz, 2H), 7.13-7.09 (m, 3H), 6.85 (d, J=7.2 Hz, 2H), 4.97-4.87 (m, 1H), 4.70-4.65 (m, 4H), 3.95-3.62 (m, 2H), 3.22-3.00 (m, 2H), 2.92-2.72 (m, 5H), 1.58 (t, J=7.2 Hz, 3H).

### Example 4

### 3-(2-(4-chlorobenzoyl)-1,2,3,4-tetrahydroisoquinolin-7-yl)-3-(1-ethyl-4-methyl-1H-benzo[d][1,2,3]triazol-5-yl)propanoic acid;

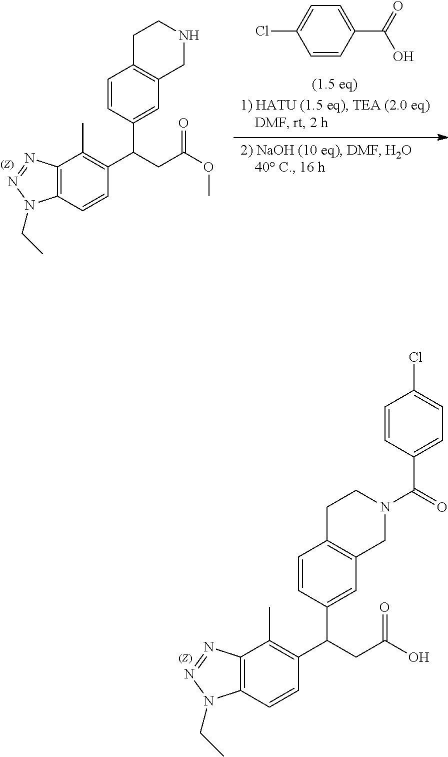

Synthesis of 3-(2-(4-chlorobenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 1. The crude product was purified by prep-HPLC (MeCN/water with 0.05% HCOOH as mobile phase) to give 3-(2-(4-chlorobenzoyl)-1,2,3,4-tetrahydroisoquinolin-7-yl)-3-(1-ethyl-4-methyl-1H-benzo[d][1,2,3]triazol-5-yl)propanoic acid; (17 mg, yield: 17%) as white solid. ESI-MS (M+H)⁺: 503.2. ¹H NMR (400 MHz, CD₃OD) δ: 7.54-7.47 (m, 6H), 7.17-7.13 (m, 3H), 5.01-5.00 (m, 1H), 4.97-4.55 (m, 4H), 3.93-3.92 (m, 1H), 3.62-3.61 (m, 1H), 3.15-2.77 (m, 7H), 1.60-1.56 (m, 3H).

### Example 5

### 3-(2-(4-(tert-butyl)benzoyl)-1,2,3,4-tetrahydroisoquinolin-7-yl)-3-(1-ethyl-4-methyl-1H-benzo[d][1,2,3]triazol-5-yl)propanoic acid;

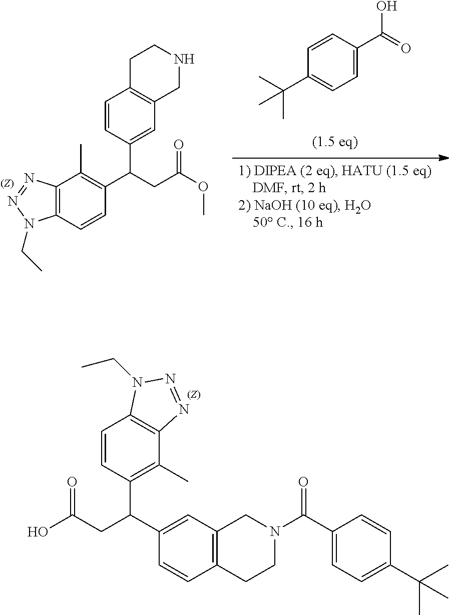

Synthesis of 3-(2-(4-(tert-butyl)benzoyl)-1,2,3,4-tetrahydroisoquinolin-7-yl)-3-(1-ethyl-4-methyl-1H-benzo[d][1,2,3]triazol-5-yl)propanoic acid; was similar to that of Example 1. The crude product was purified by prep-HPLC (MeCN/water with 0.05% HCOOH as mobile phase) to give 3-(2-(4-(tert-butyl)benzoyl)-1,2,3,4-tetrahydroisoquinolin-7-yl)-3-(1-ethyl-4-methyl-1H-benzo[d][1,2,3]triazol-5-yl)propanoic acid; as white solid (72 mg, yield: 65%). ESI-MS (M+H)⁺: 525.3. ¹H NMR (400 MHz, CD₃OD) δ: 7.56-7.47 (m, 4H), 7.42-7.34 (m, 2H), 7.17-6.90 (m, 3H), 5.01-4.96 (m, 1H), 4.80 (s, 1H), 4.74-4.65 (m, 2H), 4.60 (s, 1H), 3.92 (s, 1H), 3.64 (s, 1H), 3.16-3.04 (m, 2H), 2.92 (s, 1H), 2.82-2.77 (m, 4H), 1.63-1.57 (m, 3H), 1.36 (s, 9H).

### Example 6

### 3-(2-(cyclohexanecarbonyl)-1,2,3,4-tetrahydroisoquinolin-7-yl)-3-(1-ethyl-4-methyl-1H-benzo[d][1,2,3]triazol-5-yl)propanoic acid;

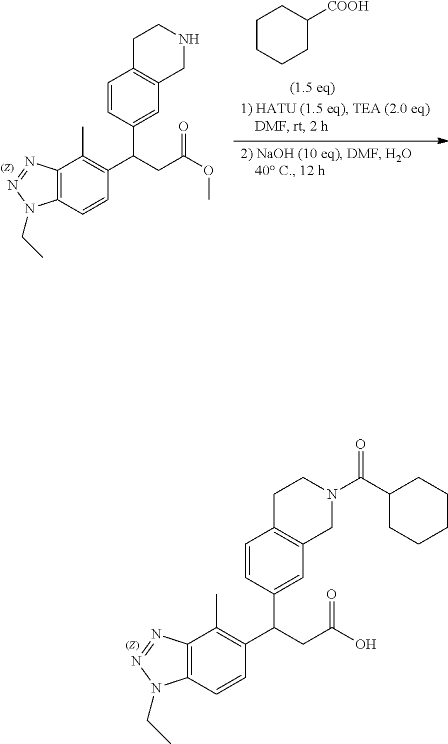

Synthesis of 3-(2-(cyclohexanecarbonyl)-1,2,3,4-tetrahydroisoquinolin-7-yl)-3-(1-ethyl-4-methyl-1H-benzo[d][1,2,3]triazol-5-yl)propanoic acid; was similar to that of Example 1. The residue was purified by prep-HPLC (MeCN/water with 0.05% HCOOH as mobile phase) to give 3-(2-(cyclohexanecarbonyl)-1,2,3,4-tetrahydroisoquinolin-7-yl)-3-(1-ethyl-4-methyl-1H-benzo[d][1,2,3]triazol-5-yl)propanoic acid; (35 mg, yield: 27%) as white solid. ESI-MS (M+H)⁺: 475.2. ¹H NMR (400 MHz, CDCl₃) δ: 7.33-7.32 (m, 2H), 7.07-6.93 (m, 3H), 4.99-4.93 (m, 1H), 4.66-4.57 (m, 4H), 3.77-3.65 (m, 2H), 3.20-3.01 (m, 2H), 2.85-2.82 (m, 4H), 2.76-2.74 (m, 1H), 2.54-2.49 (m, 1H), 1.79-1.73 (m, 4H), 1.59 (t, J=7.6 Hz, 3H), 1.54-1.48 (m, 2H), 1.29-1.24 (m, 4H).

### Example 7

### 3-(2-(2-chlorobenzoyl)-1,2,3,4-tetrahydroisoquinolin-7-yl)-3-(1-ethyl-4-methyl-1H-benzo[d][1,2,3]triazol-5-yl)propanoic acid;

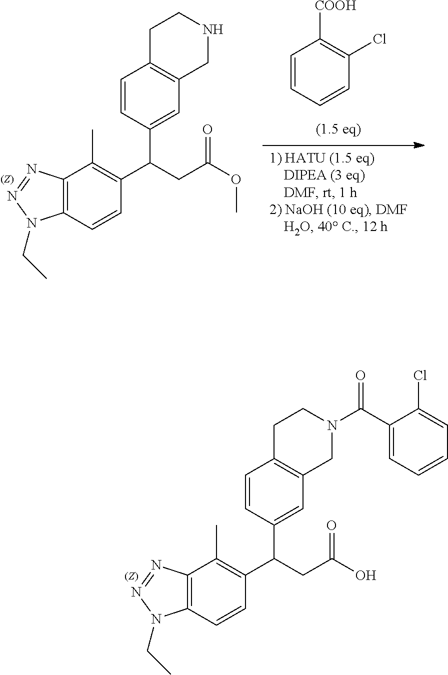

Synthesis of 3-(2-(2-chlorobenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 1. The residue was purified by prep-HPLC (MeCN/water with 0.05% HCOOH as mobile phase) to give 3-(2-(2-chlorobenzoyl)-1,2,3,4-tetrahydroisoquinolin-7-yl)-3-(1-ethyl-4-methyl-1H-benzo[d][1,2,3]triazol-5-yl)propanoic acid; as yellow solid (32 mg, yield: 22%). ESI-MS (M+H)⁺: 503.2. ¹H NMR (400 MHz, CD₃OD) δ: 7.58-7.39 (m, 6H), 7.14-6.88 (m, 3H), 4.91-4.89 (m, 1H), 4.73-4.69 (m, 2H), 4.38-4.36 (m, 1H), 3.98-3.95 (m, 1H), 3.46-3.48 (m, 1H), 3.16-2.95 (m, 3H), 2.83-2.75 (m, 5H), 1.62-1.56 (m, 3H).

### Example 8

### 3-(2-(2,4-dichlorobenzoyl)-1,2,3,4-tetrahydroisoquinolin-7-yl)-3-(1-ethyl-4-methyl-1H-benzo[d][1,2,3]triazol-5-yl)propanoic acid;

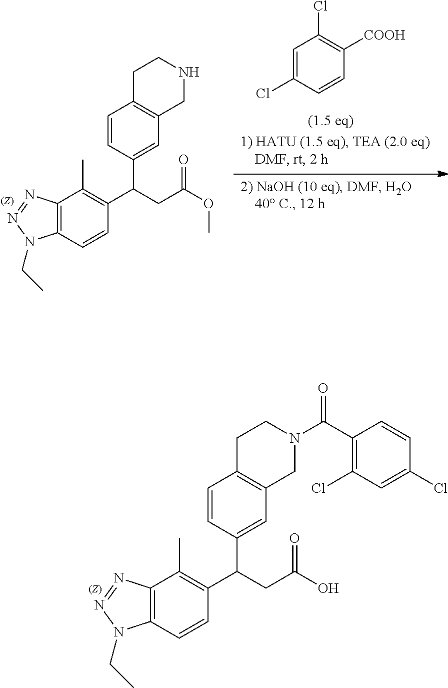

Synthesis of 3-(2-(2,4-dichlorobenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 1. The residue was purified by prep-HPLC (CH₃CN/water with 0.05% HCOOH as mobile phase) to give 3-(2-(2,4-dichlorobenzoyl)-1,2,3,4-tetrahydroisoquinolin-7-yl)-3-(1-ethyl-4-methyl-1H-benzo[d][1,2,3]triazol-5-yl)propanoic acid; (21 mg, yield: 14%) as white solid. ESI-MS (M+H)⁺: 537.1. ¹H NMR (400 MHz, CDCl₃) δ: 7.44-7.42 (m, 1H), 7.34-7.20 (m, 4H), 7.08-6.73 (m, 3H), 4.98-4.73 (m, 2H), 4.67-4.60 (m, 2H), 4.40-4.20 (m, 1H), 4.10-3.79 (m, 1H), 3.46-3.40 (m, 1H), 3.20-2.99 (m, 2H), 2.90 (t, J=5.6 Hz, 1H), 2.83-2.80 (m, 3H), 2.75-2.71 (m, 1H), 1.62-1.56 (m, 3H).

### Example 9

### 3-(2-(2,5-dichlorobenzoyl)-1,2,3,4-tetrahydroisoquinolin-7-yl)-3-(1-ethyl-4-methyl-1H-benzo[d][1,2,3]triazol-5-yl)propanoic acid;

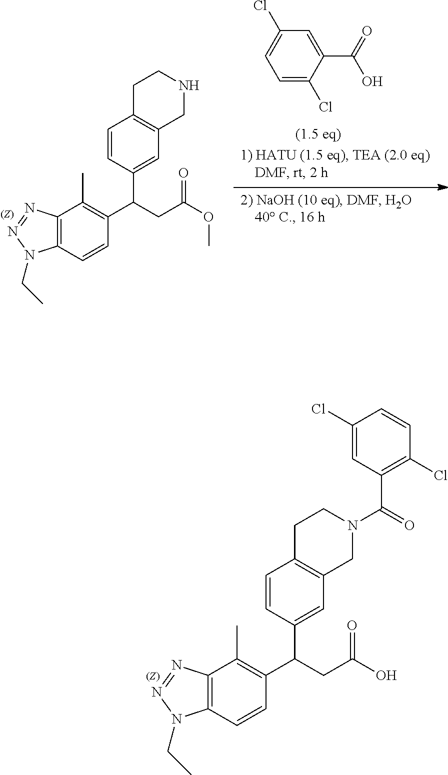

Synthesis of 3-(2-(2,5-dichlorobenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 1. The crude product was purified by prep-HPLC (CH₃CN/water with 0.05% HCOOH as mobile phase) to give 3-(2-(2,5-dichlorobenzoyl)-1,2,3,4-tetrahydroisoquinolin-7-yl)-3-(1-ethyl-4-methyl-1H-benzo[d][1,2,3]triazol-5-yl)propanoic acid; (27 mg, yield: 23%) as white solid. ESI-MS (M+H)⁺: 537.1. ¹H NMR (400 MHz, CD₃OD) δ: 7.56-7.38 (m, 5H), 7.18-6.92 (m, 3H), 5.02-4.98 (m, 1H), 4.86-4.65 (m, 3H), 4.39-4.38 (m, 1H), 4.03-3.84 (m, 1H), 3.49-3.45 (m, 1H), 3.20-3.03 (m, 2H), 2.95-2.92 (m, 1H), 2.81-2.76 (m, 4H), 1.60-1.54 (m, 3H).

### Example 10

### 3-(2-(3-chlorobenzoyl)-1,2,3,4-tetrahydroisoquinolin-7-yl)-3-(1-ethyl-4-methyl-1H-benzo[d][1,2,3]triazol-5-yl)propanoic acid;

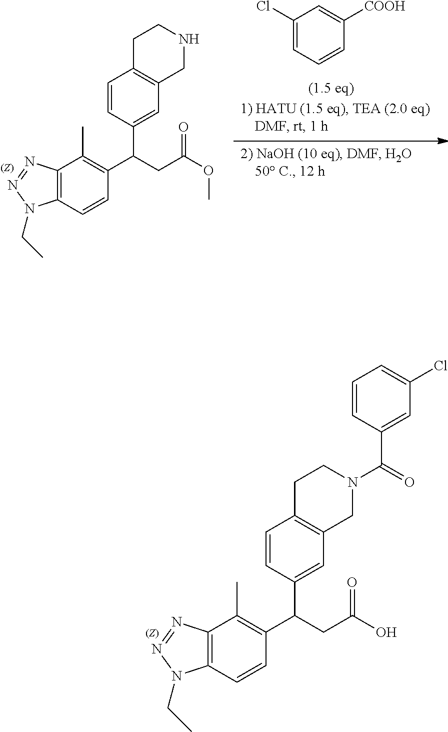

Synthesis of 3-(2-(3-chlorobenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 1. The crude product was purified by prep-HPLC (MeCN/water with 0.05% HCOOH as mobile phase) to give 3-(2-(3-chlorobenzoyl)-1,2,3,4-tetrahydroisoquinolin-7-yl)-3-(1-ethyl-4-methyl-1H-benzo[d][1,2,3]triazol-5-yl)propanoic acid; as white solid (26 mg, yield: 24%). ESI-MS (M+H)⁺: 503.2. ¹H NMR (400 MHz, CDCl₃) δ: 7.51-7.26 (m, 6H), 7.13-6.95 (m, 3H), 5.00-4.86 (m, 1H), 4.83-4.30 (m, 4H), 3.99-3.80 (m, 1H), 3.65-3.53 (m, 1H), 3.23-3.00 (m, 2H), 2.98-2.57 (m, 5H), 1.60 (t, J=6.4 Hz, 3H).

### Example 11

### 3-(2-(3,4-dichlorobenzoyl)-1,2,3,4-tetrahydroisoquinolin-7-yl)-3-(1-ethyl-4-methyl-1H-benzo[d][1,2,3]triazol-5-yl)propanoic acid;

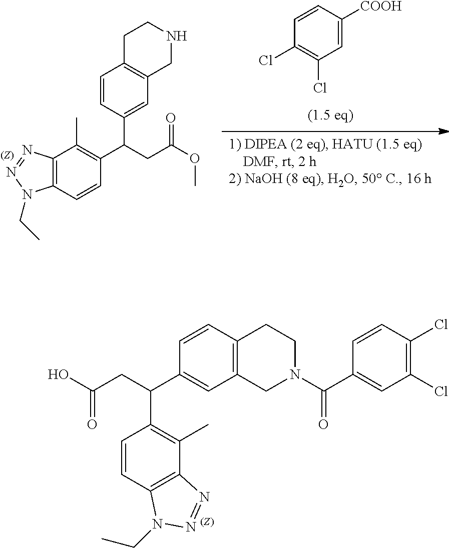

Synthesis of 3-(2-(3,4-dichlorobenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 1. The crude product was purified by prep-HPLC (MeCN/water with 0.05% HCOOH as mobile phase) to give 3-(2-(3,4-dichlorobenzoyl)-1,2,3,4-tetrahydroisoquinolin-7-yl)-3-(1-ethyl-4-methyl-1H-benzo[d][1,2,3]triazol-5-yl)propanoic acid; as white solid (84 mg, yield: 59%). ESI-MS (M+H)⁺: 537.1. ¹H NMR (400 MHz, CD₃OD) δ: 7.65-7.61 (m, 2H), 7.56-7.7.47 (m, 2H), 7.40-7.35 (m, 1H), 7.16-6.93 (m, 3H), 5.02-4.99 (m, 1H), 4.79 (s, 1H), 4.72-4.69 (m, 2H), 4.55 (s, 1H), 3.91 (s, 1H), 3.61 (s, 1H), 3.15-3.05 (m, 2H), 2.92 (s, 1H), 2.84-2.76 (m, 4H), 1.59-1.55 (m, 3H).

### Example 12

### (S)-3-(2-(3,4-dichlorobenzoyl)-1,2,3,4-tetrahydroisoquinolin-7-yl)-3-(1-ethyl-4-methyl-1H-benzo[d][1,2,3]triazol-5-yl)propanoic acid; and (R)-3-(2-(3,4-dichlorobenzoyl)-1,2,3,4-tetrahydroisoquinolin-7-yl)-3-(1-ethyl-4-methyl-1H-benzo[d][1,2,3]triazol-5-yl)propanoic acid;

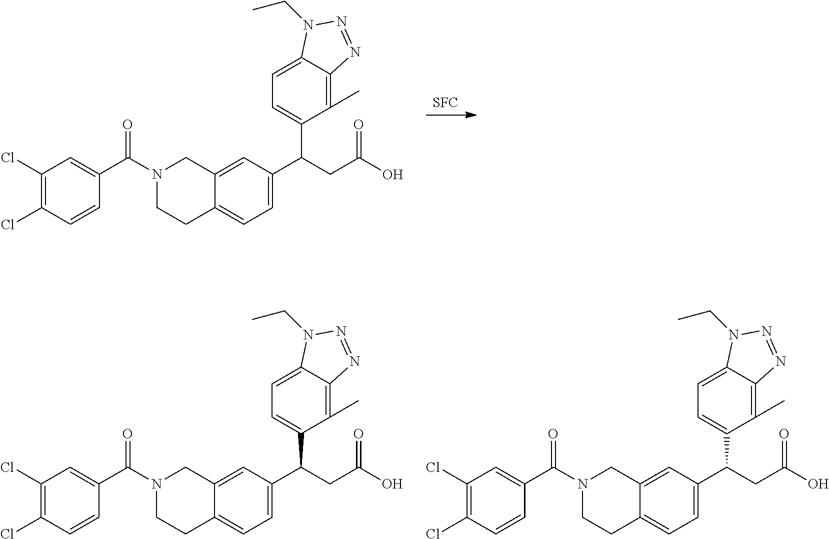 [SMILES: CCn1nnc2c(C)c(C(CC(=O)O)c3ccc4c(c3)CN(C(=O)c3ccc(Cl)c(Cl)c3)CC4)ccc21 ; "SFC" ; CCn1nnc2c(C)c([C@@H](CC(=O)O)c3ccc4c(c3)CN(C(=O)c3ccc(Cl)c(Cl)c3)CC4)ccc21 ; CCn1nnc2c(C)c([C@H](CC(=O)O)c3ccc4c(c3)CN(C(=O)c3ccc(Cl)c(Cl)c3)CC4)ccc21]

3-[2-(3,4-dichlorobenzoyl)-3,4-dihydro-1H-isoquinolin-7-yl]-3-(1-ethyl-4-methyl-benzotriazol-5-yl)propanoic acid; (164.00 mg, 305.15 umol) was separated with the following SFC conditions: (Column: CHIRALPAK AS-H 30×250 mm, 5 um; Co-solvent: 30% Methanol w/0.1% TFA in CO2 (flow rate: 100 mL/min), ABPR 120 bar, MBPR 40 psi) to give the first eluate as Ent-1 (67.00 mg, 118.43 umol, 38.81% yield, 95% purity) LCMS: Rt=1.59 min, m/z=537.1, and the second eluate as Ent-2 (78.00 mg, 137.88 umol, 45.18% yield, 95% purity). LCMS: Rt=1.59 min, m/z=537.1.

### Example 13

### 3-(2-(3,5-dichlorobenzoyl)-1,2,3,4-tetrahydroisoquinolin-7-yl)-3-(1-ethyl-4-methyl-1H-benzo[d][1,2,3]triazol-5-yl)propanoic acid;

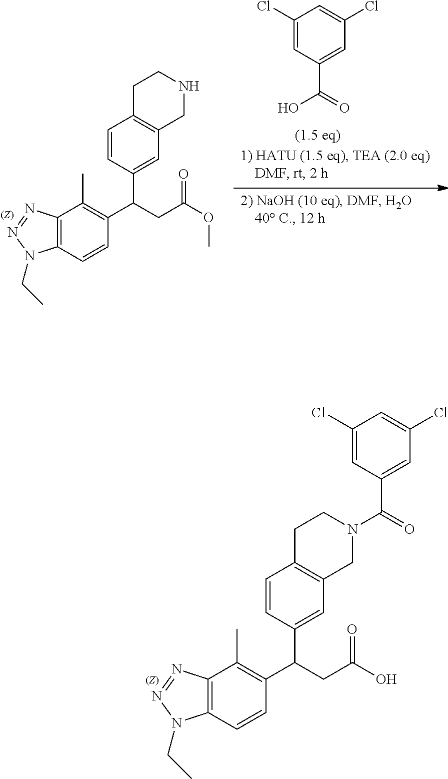

Synthesis of 3-(2-(3,5-dichlorobenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 1. The crude product was purified by prep-HPLC (MeCN/water with 0.05% HCOOH as mobile phase) to give 3-(2-(3,5-dichlorobenzoyl)-1,2,3,4-tetrahydroisoquinolin-7-yl)-3-(1-ethyl-4-methyl-1H-benzo[d][1,2,3]triazol-5-yl)propanoic acid; as white solid (35 mg, yield: 29%). ESI-MS (M+H)⁺: 537.1. ¹H NMR (400 MHz, CDCl₃) δ: 7.46-7.25 (m, 4H), 7.20-6.88 (m, 3H), 5.01-4.83 (m, 1H), 4.84-4.33 (m, 4H), 4.04-3.76 (m, 1H), 3.71-3.27 (m, 1H), 3.28-2.49 (m, 7H), 1.59 (s, 3H).

### Example 14

### 3-(1-ethyl-4-methyl-1H-benzo[d][1,2,3]triazol-5-yl)-3-(2-(pyrimidine-2-carbonyl)-1,2,3,4-tetrahydroisoquinolin-7-yl)propanoic acid;

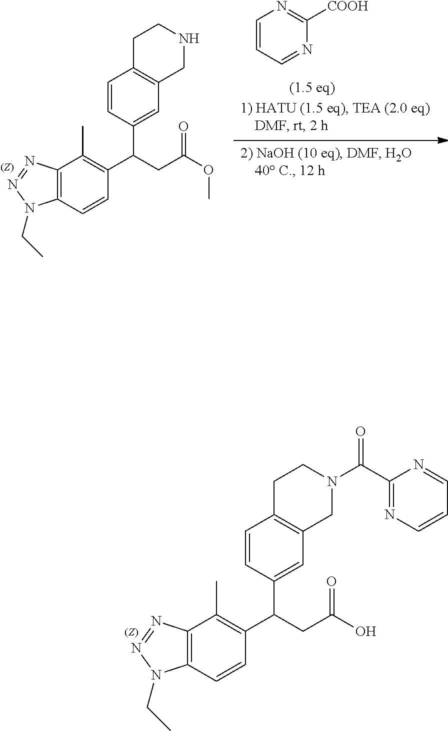

Synthesis of 3-(1-ethyl-4-methyl-1H-benzo[d][1,2,3]triazol-5-yl)-3-(2-(pyrimidine-2-carbonyl)-1,2,3,4-tetrahydroisoquinolin-7-yl)propanoic acid; was similar to that of Example 1. The residue was purified by prep-HPLC (CH₃CN/water with 0.05% HCOOH as mobile phase) to give 3-(1-ethyl-4-methyl-1H-benzo[d][1,2,3]triazol-5-yl)-3-(2-(pyrimidine-2-carbonyl)-1,2,3,4-tetrahydroisoquinolin-7-yl)propanoic acid; (30 mg, yield: 30%) as white solid. ESI-MS (M+H)⁺: 471.2. ¹H NMR (400 MHz, CD₃OD) δ: 8.82 (brs, 2H), 7.60-7.42 (m, 3H), 7.19-6.90 (m, 3H), 5.03-4.91 (m, 2H), 4.73-4.48 (m, 3H), 3.98-3.52 (m, 2H), 3.21-2.94 (m, 2H), 2.88-2.86 (m, 2H), 2.82-2.76 (m, 3H), 1.61-1.55 (m, 3H).

### Example 15

### 3-(1-ethyl-4-methyl-1H-benzo[d][1,2,3]triazol-5-yl)-3-(2-(pyrazine-2-carbonyl)-1,2,3,4-tetrahydroisoquinolin-7-yl)propanoic acid;

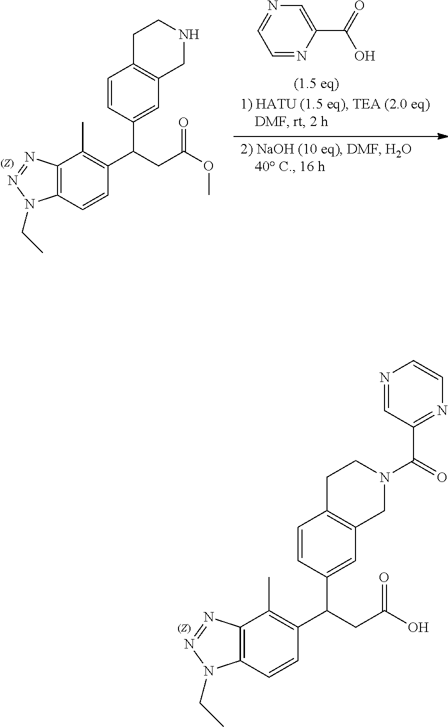

Synthesis of 3-(1-ethyl-4-methyl-1H-benzo[d][1,2,3]triazol-5-yl)-3-(2-(pyrazine-2-carbonyl)-1,2,3,4-tetrahydroisoquinolin-7-yl)propanoic acid; was similar to that of Example 1. The crude product was purified by prep-HPLC (CH3CN/water with 0.05% HCOOH as mobile phase) to give 3-(1-ethyl-4-methyl-1H-benzo[d][1,2,3]triazol-5-yl)-3-(2-(pyrazine-2-carbonyl)-1,2,3,4-tetrahydroisoquinolin-7-yl)propanoic acid; (26 mg, yield: 26%) as white solid. ESI-MS (M+H)+: 471.2. 1H NMR (400 MHz, CD3OD) δ: 8.89-8.86 (m, 1H), 8.72-8.68 (m, 2H), 7.58-7.44 (m, 2H), 7.18-6.94 (m, 3H), 5.03-4.85 (m, 2H), 4.73-4.66 (m, 3H), 3.98-3.73 (m, 2H), 3.17-2.96 (m, 4H), 2.82-2.76 (m, 3H), 1.61-1.55 (m, 3H).

### Example 16

### 3-(1-ethyl-4-methyl-1H-benzo[d][1,2,3]triazol-5-yl)-3-(2-(pyrimidine-4-carbonyl)-1,2,3,4-tetrahydroisoquinolin-7-yl)propanoic acid;

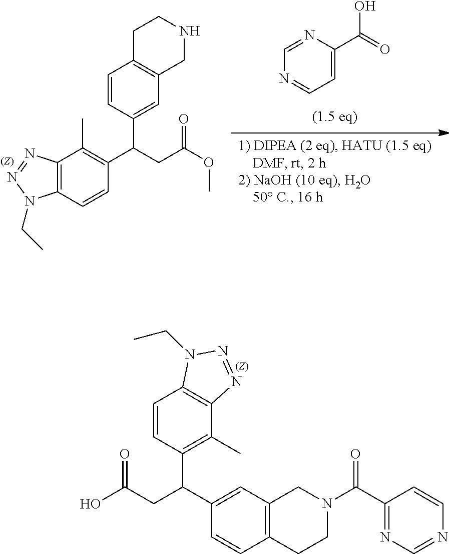

Synthesis of 3-(1-ethyl-4-methyl-1H-benzo[d][1,2,3]triazol-5-yl)-3-(2-(pyrimidine-4-carbonyl)-1,2,3,4-tetrahydroisoquinolin-7-yl)propanoic acid; was similar to that of Example 1. The crude product was purified by prep-HPLC (MeCN/water with 0.05% HCOOH as mobile phase) to give 3-(1-ethyl-4-methyl-1H-benzo[d][1,2,3]triazol-5-yl)-3-(2-(pyrimidine-4-carbonyl)-1,2,3,4-tetrahydroisoquinolin-7-yl)propanoic acid; as white solid (55 mg, yield: 55%). ESI-MS (M+H)⁺: 471.2. ¹H NMR (400 MHz, CD₃OD) δ: 9.25-9.24 (m, 1H), 8.97-8.93 (m, 1H), 7.71-7.64 (m, 3H), 7.17-6.34 (m, 3H), 5.04-4.93 (m, 1H), 4.92 (s, 1H), 4.74-4.66 (m, 2H), 4.63 (s, 1H), 3.96-3.93 (m, 1H), 3.69-3.66 (m, 1H), 3.18-3.00 (m, 2H), 2.96-2.88 (m, 2H), 2.82 (s, 2H), 2.76 (s, 1H), 1.61-1.55 (m, 3H).

### Example 17

### 3-(1-ethyl-4-methyl-1H-benzo[d][1,2,3]triazol-5-yl)-3-(2-picolinoyl-1,2,3,4-tetrahydroisoquinolin-7-yl)propanoic acid;

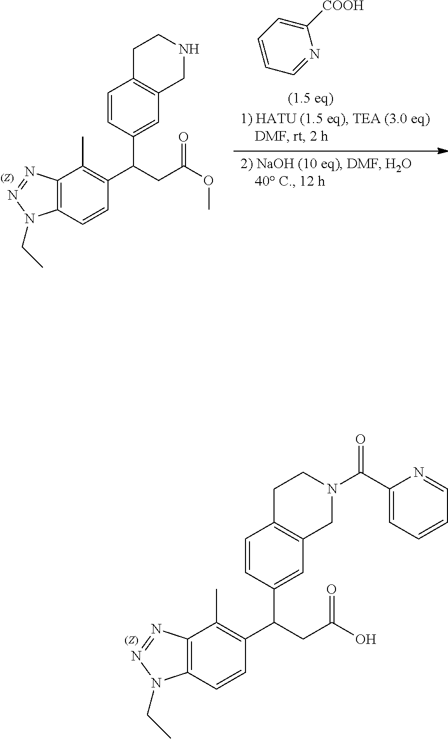

Synthesis of 3-(1-ethyl-4-methyl-1H-benzo[d][1,2,3]triazol-5-yl)-3-(2-picolinoyl-1,2,3,4-tetrahydroisoquinolin-7-yl)propanoic acid; was similar to that of Example 1. The crude product was purified by prep-HPLC (CH₃CN/H₂O with 0.05% HCOOH as mobile phase) to give 3-(1-ethyl-4-methyl-1H-benzo[d][1,2,3]triazol-5-yl)-3-(2-picolinoyl-1,2,3,4-tetrahydroisoquinolin-7-yl)propanoic acid; as yellow solid (35 mg, yield: 40%). ESI-MS (M+H)⁺: 470.2. ¹H NMR (400 MHz, CD₃OD) δ: 8.62 (s, 1H), 8.00-7.94 (m, 1H), 7.65-7.44 (m, 4H), 7.18-6.89 (m, 3H), 5.05-4.99 (m, 1H), 4.91-4.85 (m, 1H), 4.75-4.60 (m, 3H), 3.99-3.64 (m, 2H), 3.20-2.76 (m, 7H), 1.62-1.56 (m, 3H).

### Example 18

### 3-(1-ethyl-4-methyl-1H-benzo[d][1,2,3]triazol-5-yl)-3-(2-nicotinoyl-1,2,3,4-tetrahydroisoquinolin-7-yl)propanoic acid;

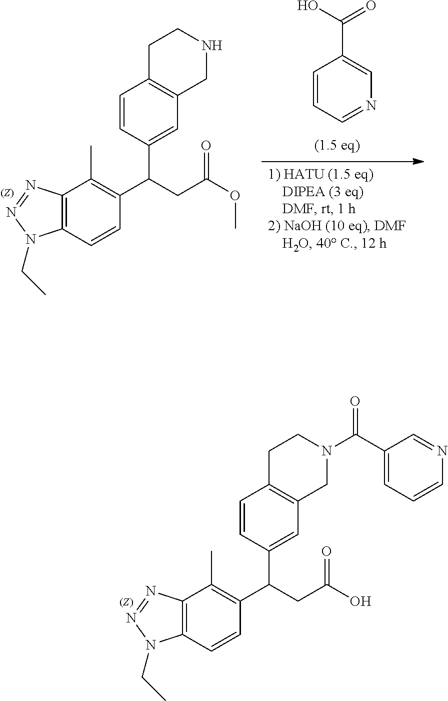

Synthesis of 3-(1-ethyl-4-methyl-1H-benzo[d][1,2,3]triazol-5-yl)-3-(2-nicotinoyl-1,2,3,4-tetrahydroisoquinolin-7-yl)propanoic acid; was similar to that of Example 1. The residue was purified by prep-HPLC (CH₃CN/H₂O with 0.05% HCOOH as mobile phase) to give 3-(1-ethyl-4-methyl-1H-benzo[d][1,2,3]triazol-5-yl)-3-(2-nicotinoyl-1,2,3,4-tetrahydroisoquinolin-7-yl)propanoic acid; as yellow solid (66 mg, yield: 42%). ESI-MS (M+H)⁺: 470.2. ¹H NMR (400 MHz, CD₃OD) δ: 8.67 (s, 2H), 7.94 (s, 1H), 7.57-7.54 (m, 3H), 7.19-7.14 (m, 3H), 4.89-4.83 (m, 1H), 4.73-4.59 (m, 4H), 3.96-3.95 (m, 1H), 3.65-3.64(m, 1H), 2.96-2.87 (m, 2H), 2.83-2.76 (m, 5H), 1.60 (m, 3H).

### Example 19

### 3-(1-ethyl-4-methyl-1H-benzo[d][1,2,3]triazol-5-yl)-3-(2-isonicotinoyl-1,2,3,4-tetrahydroisoquinolin-7-yl)propanoic acid;

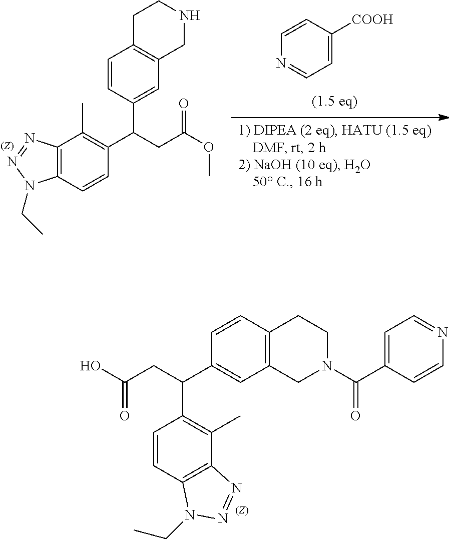

Synthesis of 3-(1-ethyl-4-methyl-1H-benzo[d][1,2,3]triazol-5-yl)-3-(2-isonicotinoyl-1,2,3,4-tetrahydroisoquinolin-7-yl)propanoic acid; was similar to that of Example 1. The residue was purified by prep-HPLC (MeCN/water with 0.05% HCOOH as mobile phase) to give 3-(1-ethyl-4-methyl-1H-benzo[d][1,2,3]triazol-5-yl)-3-(2-isonicotinoyl-1,2,3,4-tetrahydroisoquinolin-7-yl)propanoic acid; as white solid (88 mg, yield: 71%). ESI-MS (M+H)⁺: 470.1. ¹H NMR (400 MHz, CD₃OD) δ: 8.70-8.67 (m, 2H), 7.58-7.46 (m, 4H), 7.17-6.92 (m, 3H), 5.02-4.92 (m, 1H), 4.82 (s, 1H), 4.72-4.68 (m, 2H), 4.51 (s, 1H), 3.96-3.93 (m, 1H), 3.58-3.55 (m, 1H), 3.17-3.03 (m, 2H), 2.95-2.92 (m, 1H), 2.85-2.81 (m, 3H), 2.76 (s, 1H), 1.61-1.55 (m, 3H).

### Example 20

### 3-(1-ethyl-4-methyl-1H-benzo[d][1,2,3]triazol-5-yl)-3-(2-(1-methyl-1H-pyrazole-4-carbonyl)-1,2,3,4-tetrahydroisoquinolin-7-yl)propanoic acid;

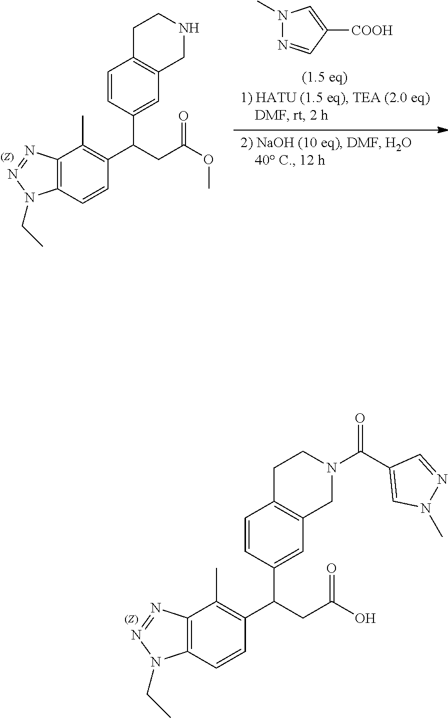

Synthesis of 3-(1-ethyl-4-methyl-1H-benzo[d][1,2,3]triazol-5-yl)-3-(2-(1-methyl-1H-pyrazole-4-carbonyl)-1,2,3,4-tetrahydroisoquinolin-7-yl)propanoic acid; was similar to that of Example 1. The residue was purified by prep-HPLC (CH₃CN/water with 0.05% HCOOH as mobile phase) to give 3-(1-ethyl-4-methyl-1H-benzo[d][1,2,3]triazol-5-yl)-3-(2-(1-methyl-1H-pyrazole-4-carbonyl)-1,2,3,4-tetrahydroisoquinolin-7-yl)propanoic acid; (30 mg, yield: 30%) as white solid. ESI-MS (M+H)⁺: 473.2. ¹H NMR (400 MHz, CD₃OD) δ: 8.01 (s, 1H), 7.79 (s, 1H), 7.56-7.49 (m, 2H), 7.13-7.08 (m, 3H), 4.98 (t, J=7.2 Hz, 1H), 4.87-4.76 (m, 2H), 4.71 (q, J=7.2 Hz, 2H), 3.94 (s, 3H), 3.88 (t, J=5.6 Hz, 2H), 3.15-3.06 (m, 2H), 2.93-2.89 (m, 2H), 2.80 (s, 3H), 1.58 (t, J=7.2 Hz, 3H).

### Example 21

### 3-(1-ethyl-4-methyl-1H-benzo[d][1,2,3]triazol-5-yl)-3-(2-(1-methyl-1H-pyrazole-3-carbonyl)-1,2,3,4-tetrahydroisoquinolin-7-yl)propanoic acid;

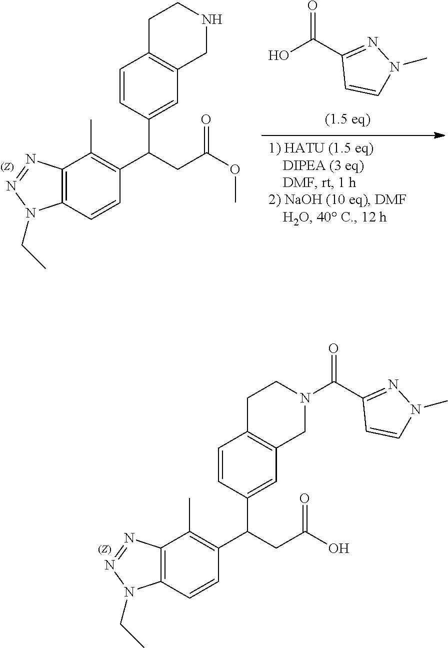

Synthesis of 3-(1-ethyl-4-methyl-1H-benzo[d][1,2,3]triazol-5-yl)-3-(2-(1-methyl-1H-pyrazole-3-carbonyl)-1,2,3,4-tetrahydroisoquinolin-7-yl)propanoic acid; was similar to that of Example 1. The residue was purified by prep-HPLC to give 3-(1-ethyl-4-methyl-1H-benzo[d][1,2,3]triazol-5-yl)-3-(2-(1-methyl-1H-pyrazole-3-carbonyl)-1,2,3,4-tetrahydroisoquinolin-7-yl)propanoic acid; as yellow solid (38 mg, yield: 25%). ESI-MS (M+H)⁺: 473.2. ¹H NMR (400 MHz, CD₃OD) δ: 7.65-7.5 (m, 3H), 7.14-7.12(m, 3H), 6.63 (s, 1H), 5.04-5.00 (m, 2H), 4.90-4.71 (m, 3H), 4.12-4.10 (m, 1H), 3.96-3.91 (m, 4H), 3.17-3.10 (m, 2H), 2.91-2.79 (m, 5H), 1.60 (t, J=7.2 Hz, 3H).

### Example 22

### 3-(1-ethyl-4-methyl-1H-benzo[d][1,2,3]triazol-5-yl)-3-(2-(1-methyl-1H-imidazole-4-carbonyl)-1,2,3,4-tetrahydroisoquinolin-7-yl)propanoic acid;

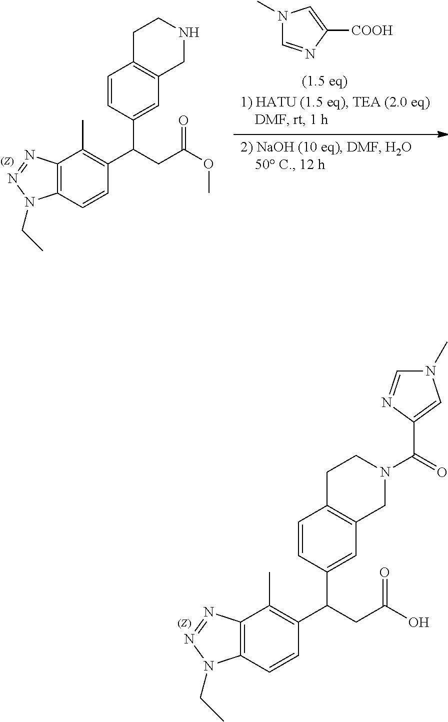

Synthesis of 3-(1-ethyl-4-methyl-1H-benzo[d][1,2,3]triazol-5-yl)-3-(2-(1-methyl-1H-imidazole-4-carbonyl)-1,2,3,4-tetrahydroisoquinolin-7-yl)propanoic acid; was similar to that of Example 1. The residue was purified by prep-HPLC to give 3-(1-ethyl-4-methyl-1H-benzo[d][1,2,3]triazol-5-yl)-3-(2-(1-methyl-1H-imidazole-4-carbonyl)-1,2,3,4-tetrahydroisoquinolin-7-yl)propanoic acid; as white solid (32 mg, yield: 32%). ESI-MS (M+H)⁺: 473.2. ¹H NMR (400 MHz, CD₃OD) δ: 8.45 (s, 1H), 7.95-7.34 (m, 4H), 7.22-7.01 (m, 2H), 5.15-4.93 (m, 2H), 4.84-4.60 (m, 3H), 4.21-3.86 (m, 2H), 3.79 (s, 3H), 3.17-2.84 (m, 4H), 2.79 (s, 3H), 1.58 (t, J=7.2 Hz, 3H).

### Example 23

### 3-(1-ethyl-4-methyl-1H-benzo[d][1,2,3]triazol-5-yl)-3-(2-(isoxazole-3-carbonyl)-1,2,3,4-tetrahydroisoquinolin-7-yl)propanoic acid;

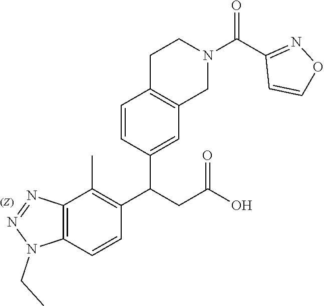 [SMILES: CCn1nnc2c(C)c(C(CC(=O)O)c3ccc4c(c3)CN(C(=O)c3ccon3)CC4)ccc21]

### 1. Preparation of methyl 3-(1-ethyl-4-methyl-1H-benzo[d][1,2,3]triazol-5-yl)-3-(2-(isoxazole-3-carbonyl)-1,2,3,4-tetrahydroisoquinolin-7-yl)propanoate

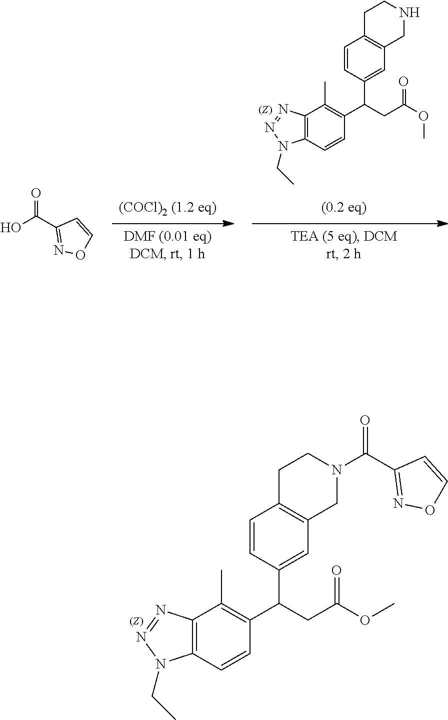 [SMILES: CCn1nnc2c(C)c(C(CC(=O)OC)c3ccc4c(c3)CNCC4)ccc21 ; O=C(O)c1ccon1 ; "DMF (0.01 eq)DCM, rt, 1 h" ; "TEA (5 eq), DCMrt, 2 h" ; CCn1nnc2c(C)c(C(CC(=O)OC)c3ccc4c(c3)CN(C(=O)c3ccon3)CC4)ccc21]

To a solution of isoxazole-3-carboxylic acid; (119 mg, 1.05 mmol) in DCM (3 mL) was added (COCl)₂ (190 mg, 1.5 mmol). Then DMF (cat) was added in the mixture. The reaction was stirred at rt for 1 h. A solution of methyl 3-(1-ethyl-4-methyl-1H-benzo[d][1,2,3]triazol-5-yl)-3-(1,2,3,4-tetrahydroisoquinolin-7-yl)propanoate (80 mg, 0.21 mmol) and TEA (530 mg, 5.25 mmol) in DCM (2 mL) was added to the mixture. The reaction was stirred at rt for another 2 h. The residue was concentrated to give methyl 3-(1-ethyl-4-methyl-1H-benzo[d][1,2,3]triazol-5-yl)-3-(2-(isoxazole-3-carbonyl)-1,2,3,4-tetrahydroisoquinolin-7-yl)propanoate (90 mg, yield: 90%) as white solid. ESI-MS (M+H)⁺: 474.2.

### 2. The Preparation of 3-(1-ethyl-4-methyl-1H-benzo[d][1,2,3]triazol-5-yl)-3-(2-(isoxazole-3-carbonyl)-1,2,3,4-tetrahydroisoquinolin-7-yl)propanoic acid;

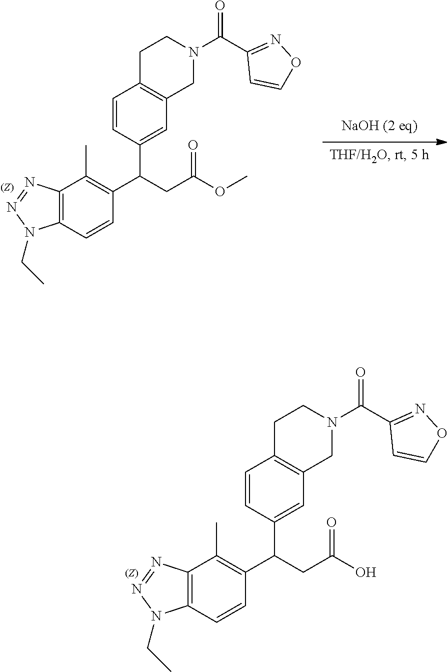 [SMILES: CCn1nnc2c(C)c(C(CC(=O)OC)c3ccc4c(c3)CN(C(=O)c3ccon3)CC4)ccc21 ; "THF/H2O, rt, 5 h" ; CCn1nnc2c(C)c(C(CC(=O)O)c3ccc4c(c3)CN(C(=O)c3ccon3)CC4)ccc21]

To a solution of methyl 3-(1-ethyl-4-methyl-1H-benzo[d][1,2,3]triazol-5-yl)-3-(2-(isoxazole-3-carbonyl)-1,2,3,4-tetrahydroisoquinolin-7-yl)propanoate (90 mg, 0.19 mmol) in THF/H₂O (4:1, 2 mL) was added NaOH (15 mg, 3.8 mmol). The reaction was stirred at rt for 5 h, concentrated. The residue was purified by prep-HPLC (CH₃CN/H₂O with 0.05% HCOOH as mobile phase) to give 3-(1-ethyl-4-methyl-1H-benzo[d][1,2,3]triazol-5-yl)-3-(2-(isoxazole-3-carbonyl)-1,2,3,4-tetrahydroisoquinolin-7-yl)propanoic acid; as yellow solid (25 mg, yield: 32%). ESI-MS (M+H)⁺: 460.2. ¹H NMR (400 MHz, CD₃OD) δ: 8.83-8.81 (m, 1H), 7.57-7.51 (m, 2H), 7.17-7.12 (m, 3H), 6.76-6.72 (m, 1H), 5.01-4.99 (m, 1H), 4.90-4.84 (m, 4H), 3.96-3.89 (m, 2H), 3.14-3.09 (m, 2H), 2.93-2.78 (m, 5H), 1.60 (m, 3H).

### Example 24

### 3-(1-ethyl-4-methyl-1H-benzo[d][1,2,3]triazol-5-yl)-3-(2-(thiazole-4-carbonyl)-1,2,3,4-tetrahydroisoquinolin-7-yl)propanoic acid;

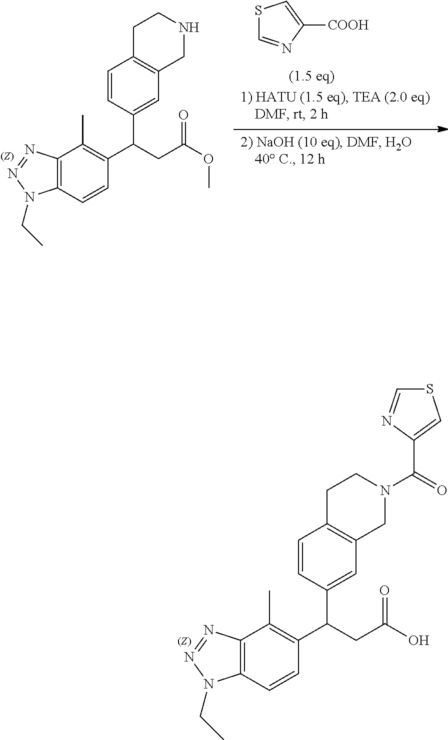

Synthesis of 3-(1-ethyl-4-methyl-1H-benzo[d][1,2,3]triazol-5-yl)-3-(2-(thiazole-4-carbonyl)-1,2,3,4-tetrahydroisoquinolin-7-yl)propanoic acid; was similar to that of Example 1. The residue was purified by prep-HPLC (CH₃CN/water with 0.05% HCOOH as mobile phase) to give 3-(1-ethyl-4-methyl-1H-benzo[d][1,2,3]triazol-5-yl)-3-(2-(thiazole-4-carbonyl)-1,2,3,4-tetrahydroisoquinolin-7-yl)propanoic acid; (26 mg, yield: 25%) as a white solid. ESI-MS (M+H)⁺: 476.2. ¹H NMR (400 MHz, CD₃OD) δ: 9.06 (s, 1H), 8.09 (s, 1H), 7.53-7.47 (m, 2H), 7.16-6.97 (m, 3H), 5.01-4.96 (m, 1H), 4.82-4.73 (m, 2H), 4.71-4.62 (m, 2H), 3.93-3.91 (m, 2H), 3.15-3.07 (m, 2H), 2.93-2.91 (m, 2H), 2.81-2.77 (m, 3H), 1.59-1.56 (m, 3H).

### Example 25

### 3-(1-ethyl-4-methyl-1H-benzo[d][1,2,3]triazol-5-yl)-3-(2-(3-methylbenzoyl)-1,2,3,4-tetrahydroisoquinolin-7-yl)propanoic acid;

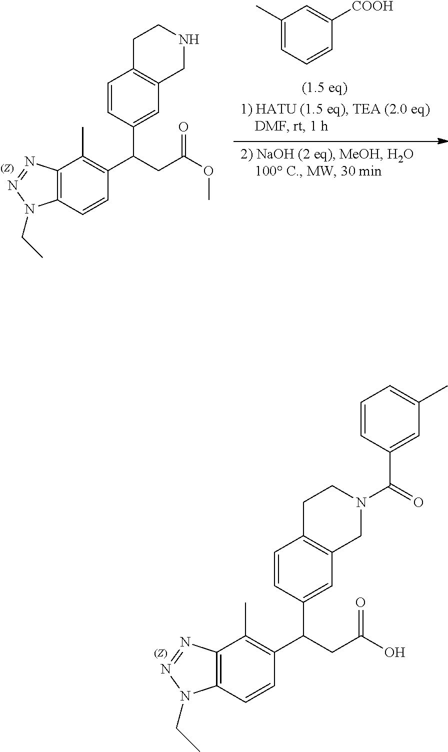

Synthesis of 3-(1-ethyl-4-methyl-1H-benzo[d][1,2,3]triazol-5-yl)-3-(2-(3-methylbenzoyl)-1,2,3,4-tetrahydroisoquinolin-7-yl)propanoic acid; was similar to that of Example 1 except hydrolyzed under microwave at 100 oC for 30 min. The residue was purified by prep-HPLC (CH₃CN/water with 0.1% CF₃COOH as mobile phase) to give 3-(1-ethyl-4-methyl-1H-benzo[d][1,2,3]triazol-5-yl)-3-(2-(3-methylbenzoyl)-1,2,3,4-tetrahydroisoquinolin-7-yl)propanoic acid; (110 mg, yield: 57%) as a white solid. ESI-MS (M+H)⁺: 483.2. ¹H NMR (400 MHz, CDCl₃) δ: 7.37 (br. s., 4H), 6.87-7.24 (m, 5H), 2.92-5.34 (m, 11H), 2.83 (br. s., 3H), 2.37 (s, 3H), 1.62 (br. s., 3H).

### Example 26

### 3-(2-(3,4-dimethylbenzoyl)-1,2,3,4-tetrahydroisoquinolin-7-yl)-3-(1-ethyl-4-methyl-1H-benzo[d][1,2,3]triazol-5-yl)propanoic acid;

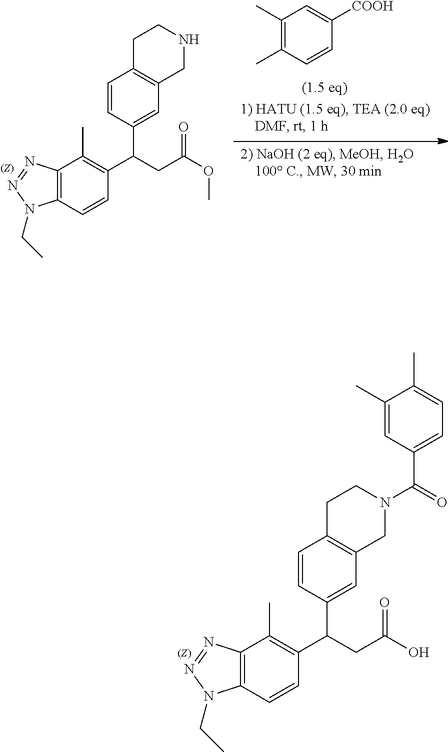

Synthesis of 3-(2-(3,4-dimethylbenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 25. The residue was purified by prep-HPLC (CH₃CN/water with 0.1% CF₃COOH as mobile phase) to give 3-(2-(3,4-dimethylbenzoyl)-1,2,3,4-tetrahydroisoquinolin-7-yl)-3-(1-ethyl-4-methyl-1H-benzo[d][1,2,3]triazol-5-yl)propanoic acid; (113 mg, yield: 57%) as a white solid. ESI-MS (M+H)⁺: 497.2. ¹H NMR (400 MHz, CDCl₃) δ: 6.77-7.60 (m, 8H), 2.98-5.87 (m, 12H), 2.82 (br. s., 3H), 2.12-2.39 (m, 6H), 1.62 (t, J=6.90 Hz, 3H).

### Example 27

### 3-(2-(2,3-dimethylbenzoyl)-1,2,3,4-tetrahydroisoquinolin-7-yl)-3-(1-ethyl-4-methyl-1H-benzo[d][1,2,3]triazol-5-yl)propanoic acid;

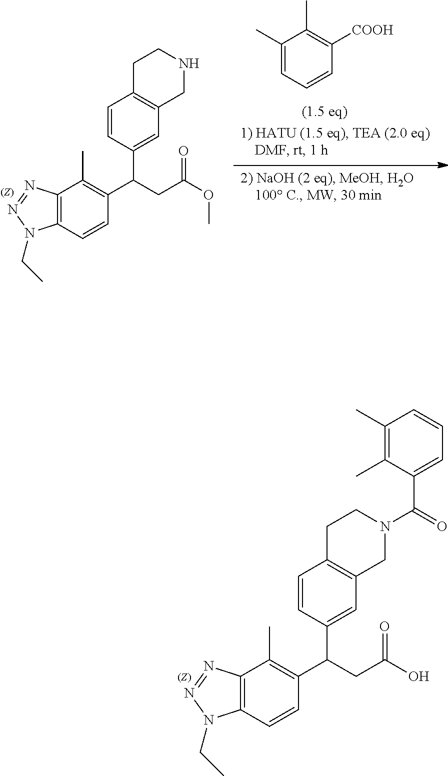

Synthesis of 3-(2-(2,3-dimethylbenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 25. The residue was purified by prep-HPLC (CH₃CN/water with 0.1% CF₃COOH as mobile phase) to give 3-(2-(2,3-dimethylbenzoyl)-1,2,3,4-tetrahydroisoquinolin-7-yl)-3-(1-ethyl-4-methyl-1H-benzo[d][1,2,3]triazol-5-yl)propanoic acid; (118 mg, yield: 65%) as a white solid. ESI-MS (M+H)⁺: 497.3. ¹H NMR (400 MHz, CDCl₃) δ: 7.29-7.49 (m, 2H), 6.48-7.24 (m, 6H), 2.51-5.11 (m, 14H), 2.00-2.39 (m, 6H), 1.47-1.73 (m, 3H).

### Example 28

### 3-(1-ethyl-4-methyl-1H-benzo[d][1,2,3]triazol-5-yl)-3-(2-(2,3,5-trimethylbenzoyl)-1,2,3,4-tetrahydroisoquinolin-7-yl)propanoic acid;

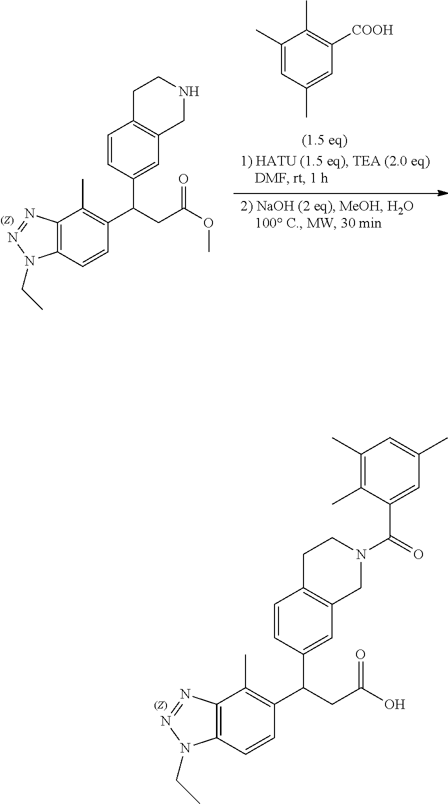

Synthesis of 3-(1-ethyl-4-methyl-1H-benzo[d][1,2,3]triazol-5-yl)-3-(2-(2,3,5-trimethylbenzoyl)-1,2,3,4-tetrahydroisoquinolin-7-yl)propanoic acid; was similar to that of Example 25. The residue was purified by prep-HPLC (CH₃CN/water with 0.1% CF₃COOH as mobile phase) to give 3-(1-ethyl-4-methyl-1H-benzo[d][1,2,3]triazol-5-yl)-3-(2-(2,3,5-trimethylbenzoyl)-1,2,3,4-tetrahydroisoquinolin-7-yl)propanoic acid; (118 mg, yield: 65%) as a white solid. ESI-MS (M+H)⁺: 511.2. ¹H NMR (400 MHz, CDCl₃) δ: 7.29-7.62 (m, 2H), 6.56-7.23 (m, 5H), 2.88-5.09 (m, 11H), 2.79 (s, 3H), 2.73 (s, 1H), 1.92-2.38 (m, 9H), 1.64 (t, J=7.28 Hz, 3H).

### Example 29

### 3-(2-(3,5-diethylbenzoyl)-1,2,3,4-tetrahydroisoquinolin-7-yl)-3-(1-ethyl-4-methyl-1H-benzo[d][1,2,3]triazol-5-yl)propanoic acid;

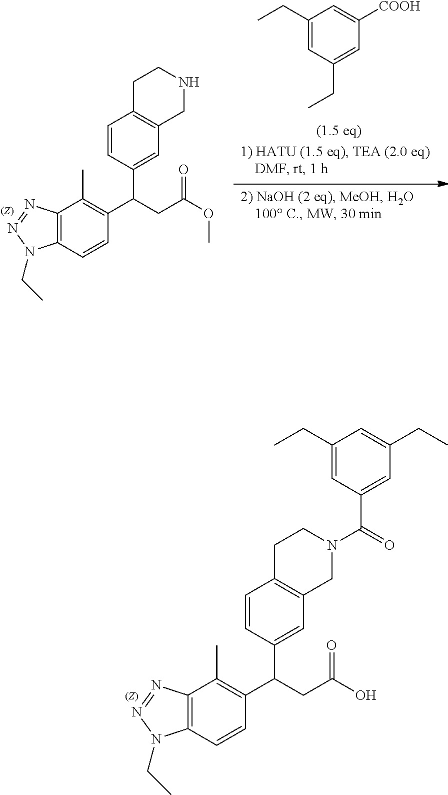

Synthesis of 3-(2-(3,5-diethylbenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 25. The residue was purified by prep-HPLC (CH₃CN/water with 0.1% CF₃COOH as mobile phase) to give 3-(2-(3,5-diethylbenzoyl)-1,2,3,4-tetrahydroisoquinolin-7-yl)-3-(1-ethyl-4-methyl-1H-benzo[d][1,2,3]triazol-5-yl)propanoic acid; (129 mg, yield: 66%) as a white solid. ESI-MS (M+H)⁺: 525.3. ¹H NMR (400 MHz, CDCl₃) δ: 7.30-7.64 (m, 2H), 6.67-7.19 (m, 6H), 2.95-5.46 (m, 12H), 2.83 (br. s., 3H), 2.64 (d, J=7.03 Hz, 4H), 1.63 (br. s., 3H), 1.23 (br. s., 6H).

### Example 30

### 3-(2-(3,5-dimethylbenzoyl)-1,2,3,4-tetrahydroisoquinolin-7-yl)-3-(1-ethyl-4-methyl-1H-benzo[d][1,2,3]triazol-5-yl)propanoic acid;

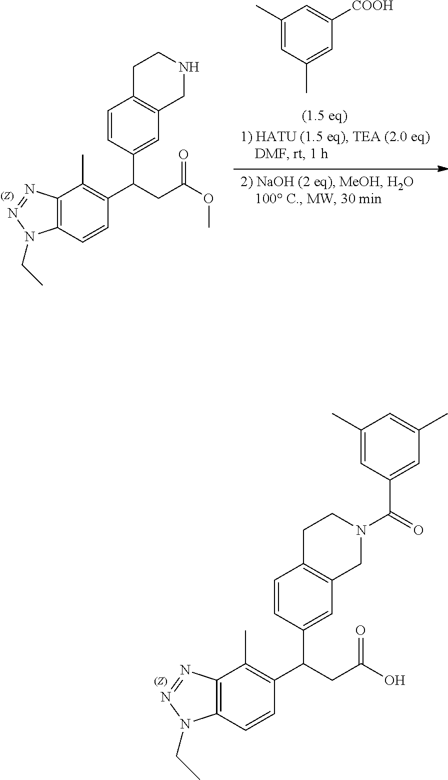

Synthesis of 3-(2-(3,5-dimethylbenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 25. The residue was purified by prep-HPLC (CH₃CN/water with 0.1% CF₃COOH as mobile phase) to give 3-(2-(3,5-dimethylbenzoyl)-1,2,3,4-tetrahydroisoquinolin-7-yl)-3-(1-ethyl-4-methyl-1H-benzo[d][1,2,3]triazol-5-yl)propanoic acid; (150 mg, yield: 61%) as a white solid. ESI-MS (M+H)⁺: 497.2. ¹H NMR (400 MHz, CDCl₃) δ: 7.38 (br. s., 2H), 6.87-7.21 (m, 6H), 2.88-5.21 (m, 11H), 2.83 (br. s., 3H), 2.33 (s, 6H), 1.62 (br. s., 3H).

### Example 31

### (S)-3-(2-(3,5-dimethylbenzoyl)-1,2,3,4-tetrahydroisoquinolin-7-yl)-3-(1-ethyl-4-methyl-1H-benzo[d][1,2,3]triazol-5-yl)propanoic acid; and (R)-3-(2-(3,5-dimethylbenzoyl)-1,2,3,4-tetrahydroisoquinolin-7-yl)-3-(1-ethyl-4-methyl-1H-benzo[d][1,2,3]triazol-5-yl)propanoic acid;

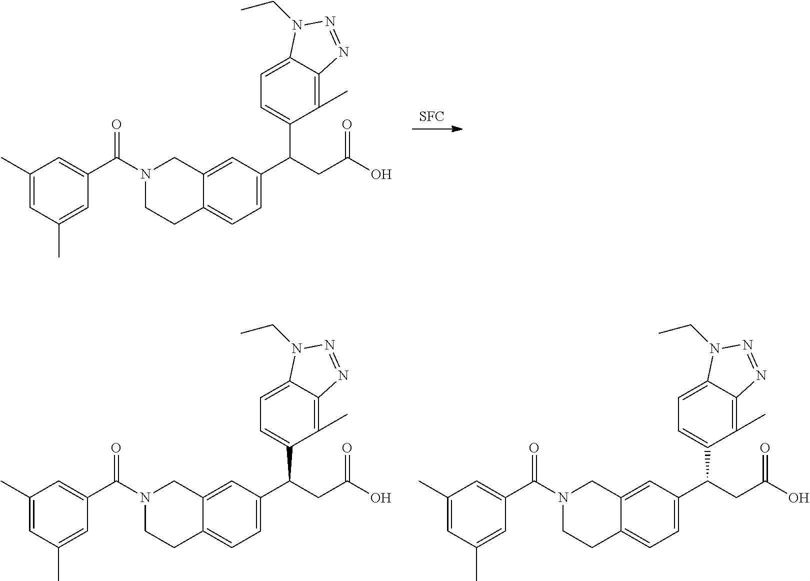

3-[2-(3,5-Dimethylbenzoyl)-3,4-dihydro-1H-isoquinolin-7-yl]-3-(1-ethyl-4-methyl-benzotriazol-5-yl)propanoic acid; (150.00 mg, 302.05 umol) was separated under the following SFC condition: (Column: 2.1×25.0 cm Chiralpak AD-H from Chiral Technologies (West Chester, Pa.); Solvent: CO₂, Co-solvent (Solvent B) Ethanol with 0.25% Isopropylamine; Isocratic Method: 54% Co-solvent at 74 g/min; System Pressure: 110 bar; Column Temperature: 25 oC. Sample Diluent: EtOH.) to give the first eluate as Ent-1 (62.40 mg, 123.77 umol, 40.98% yield, 98.5% purity). ESI-MS (M+H)⁺: 497.3. ¹H NMR (400 MHz, CDCl₃) δ: 7.38 (br. s., 2H), 6.87-7.21 (m, 6H), 2.88-5.21 (m, 11H), 2.83 (br. s., 3H), 2.33 (s, 6H), 1.62 (br. s., 3H), and the second eluate as Ent-2 (63.60 mg, 126.79 umol, 41.98% yield, 99.0% purity) LCMS: Rt=1.52 min, m/z=497.3. ¹H NMR (400 MHz, CDCl₃) δ: 7.38 (br. s., 2H), 6.87-7.21 (m, 6H), 2.88-5.21 (m, 11H), 2.83 (br. s., 3H), 2.33 (s, 6H), 1.62 (br. s., 3H).

### Example 32

### 3-(1-ethyl-4-methyl-1H-benzo[d][1,2,3]triazol-5-yl)-3-(2-(2,3,5,6-tetramethylbenzoyl)-1,2,3,4-tetrahydroisoquinolin-7-yl)propanoic acid;

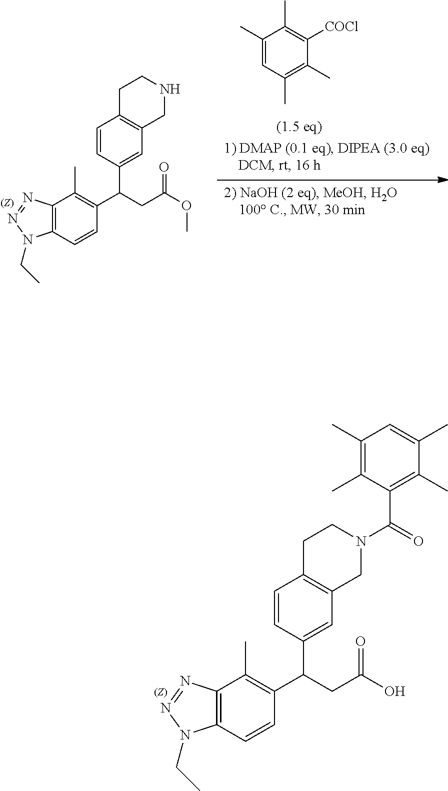

Methyl 3-(1-ethyl-4-methyl-benzotriazol-5-yl)-3-(1,2,3,4-tetrahydroisoquinolin-7-yl)propanoate (200.00 mg, 482.01 umol, Hydrochloride), 2,3,5,6-tetramethylbenzoyl chloride (94.80 mg, 482.01 umol), DMAP (11.78 mg, 96.40 umol) and DIPEA (186.88 mg, 1.45 mmol, 252.55 uL) in DCM (2.00 mL) was stirred at rt for overnight. After washing with aq NaHCO₃ and brine, the dried concentrated residue was chromatographed on Si gel (HE/EA 0-100%) to give methyl 3-(1-ethyl-4-methyl-benzotriazol-5-yl)-3-[2-(2,3,5,6-tetramethylbenzoyl)-3,4-dihydro-1H-isoquinolin-7-yl]propanoate (260.00 mg, 482.66 umol, 100% yield). ESI-MS (M+H)⁺: 539.3.

Methyl 3-(1-ethyl-4-methyl-benzotriazol-5-yl)-3-[2-(2,3,5,6-tetramethylbenzoyl)-3,4-dihydro-1H-isoquinolin-7-yl]propanoate (260.00 mg, 482.66 umol) in methanol (2.00 mL) was added sodium hydroxide (2 M, 482.66 uL) and microwaved at 100° C. for 30 min. After neutralized with 2N HCl, the crude was purified with prep HPLC (CH₃CN/water with 0.1% CF₃COOH as mobile phase) to give 13-(1-ethyl-4-methyl-benzotriazol-5-yl)-3-[2-(2,3,5,6-tetramethylbenzoyl)-3,4-dihydro-1H-isoquinolin-7-yl]propanoic acid; (107.00 mg, 178.45 umol, 36.97% yield). ESI-MS (M+H)⁺: 525.2. ¹H NMR (400 MHz, CDCl₃) δ: 7.30 (s, 2H), 6.61-7.17 (m, 4H), 4.19-5.17 (m, 5H), 2.66-4.14 (m, 9H), 1.89-2.30 (m, 12H), 1.51-1.74 (m, 3H).

### Example 33

### 3-(2-(2,6-dimethylbenzoyl)-1,2,3,4-tetrahydroisoquinolin-7-yl)-3-(1-ethyl-4-methyl-1H-benzo[d][1,2,3]triazol-5-yl)propanoic acid;

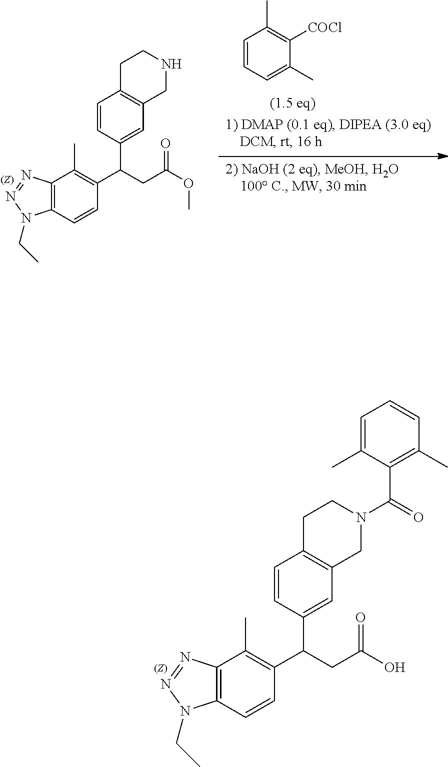

Synthesis of 3-(2-(2,6-dimethylbenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 32. The residue was purified by prep-HPLC (CH₃CN/water with 0.1% CF₃COOH as mobile phase) to give 3-(2-(2,6-dimethylbenzoyl)-1,2,3,4-tetrahydroisoquinolin-7-yl)-3-(1-ethyl-4-methyl-1H-benzo[d][1,2,3]triazol-5-yl)propanoic acid; (98.2 mg, yield: 33%) as a white solid. ESI-MS (M+H)⁺: 497.2. ¹H NMR (400 MHz, CDCl₃) δ: 7.29-7.56 (m, 2H), 6.92-7.24 (m, 5.5H), 6.69 (s, 0.5H), 4.82-5.08 (m, 2H), 4.52-4.76 (m, 2H), 3.90-4.34 (m, 2H), 2.90-3.54 (m, 4H), 2.60-2.87 (m, 4H), 1.97-2.32 (m, 6H), 1.50-1.76 (m, 3H).

### Example 34

### 3-(2-(2,6-diethylbenzoyl)-1,2,3,4-tetrahydroisoquinolin-7-yl)-3-(1-ethyl-4-methyl-1H-benzo[d][1,2,3]triazol-5-yl)propanoic acid;

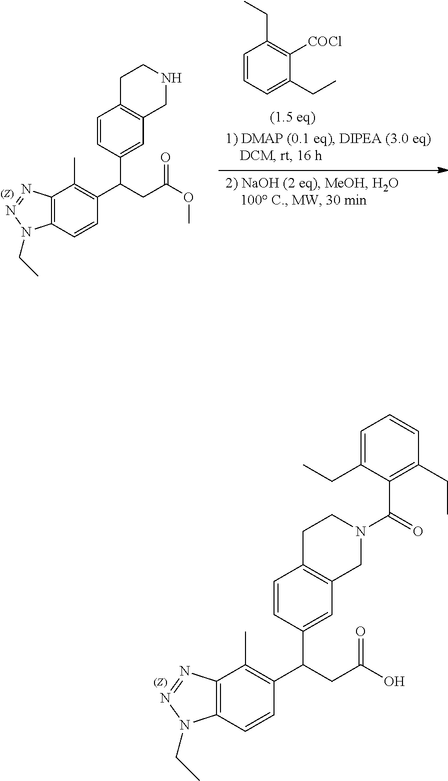

Synthesis of 3-(2-(2,6-diethylbenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 32. The residue was purified by prep-HPLC (CH₃CN/water with 0.1% CF₃COOH as mobile phase) to give 3-(2-(2,6-diethylbenzoyl)-1,2,3,4-tetrahydroisoquinolin-7-yl)-3-(1-ethyl-4-methyl-1H-benzo[d][1,2,3]triazol-5-yl)propanoic acid; (85.4 mg, yield: 35%) as a white solid. ESI-MS (M+H)⁺: 525.2. ¹H NMR (400 MHz, CDCl₃) δ: 7.27-7.64 (m, 3H), 6.52-7.23 (m, 5H), 3.90-5.42 (m, 6H), 2.22-3.52 (m, 12H), 1.61 (t, J=7.28 Hz, 3H), 0.96-1.32 (m, 6H).

### Example 35

### 3-(2-(2,6-difluorobenzoyl)-1,2,3,4-tetrahydroisoquinolin-7-yl)-3-(1-ethyl-4-methyl-1H-benzo[d][1,2,3]triazol-5-yl)propanoic acid;

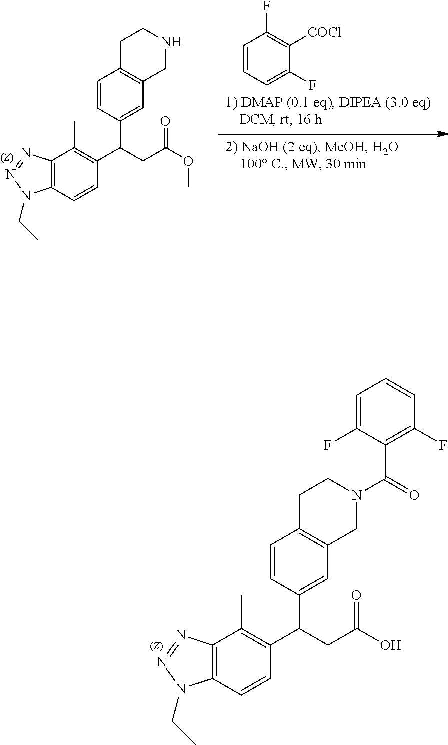

Synthesis of 3-(2-(2,6-difluorobenzoyl)-1,2,3,4-tetrahydroisoquinolin-7-yl)-3-(1-ethyl-4-methyl-1H-benzo[d][1,2,3]triazol-5-yl)propanoic acid; was similar to that of Example 32. The residue was purified by prep-HPLC (CH₃CN/water with 0.1% CF₃COOH as mobile phase) to give 3-(2-(2,6-difluorobenzoyl)-1,2,3,4-tetrahydroisoquinolin-7-yl)-3-(1-ethyl-4-methyl-1H-benzo[d][1,2,3]triazol-5-yl)propanoic acid; (135.7 mg, yield: 55%) as a white solid. ESI-MS (M+H)⁺: 505.2. ¹H NMR (400 MHz, CDCl₃) δ: 7.33-7.47 (m, 2H), 6.64-7.21 (m, 7H), 4.79-5.06 (m, 2H), 4.51-4.75 (m, 2H), 3.39-4.48 (m, 3H), 2.97-3.29 (m, 2H), 2.63-2.95 (m, 5H), 1.34-1.77 (m, 3H).

### Example 36

### 3-(2-((3r,5r,7r)-adamantane-1-carbonyl)-1,2,3,4-tetrahydroisoquinolin-7-yl)-3-(1-ethyl-4-methyl-1H-benzo[d][1,2,3]triazol-5-yl)propanoic acid;

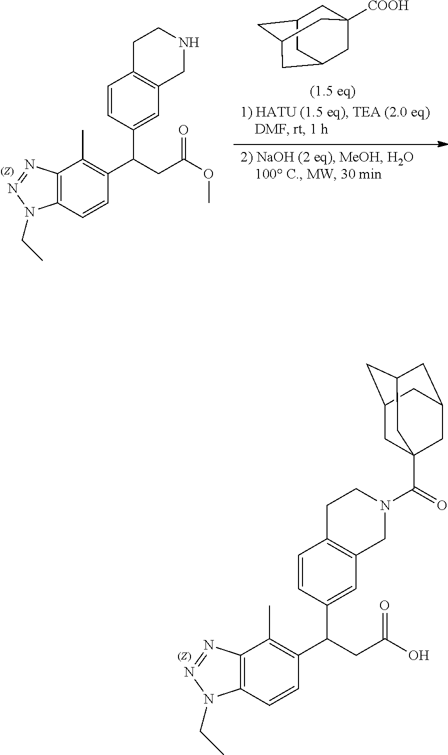

Synthesis of 3-(2-((3r,5r,7r)-adamantane-1-carbonyl)-1,2,3,4-tetrahydroisoquinolin-7-yl)-3-(1-ethyl-4-methyl-1H-benzo[d][1,2,3]triazol-5-yl)propanoic acid; was similar to that of Example 25. The residue was purified by prep-HPLC (CH₃CN/water with 0.1% CF₃COOH as mobile phase) to give 3-(2-((3r,5r,7r)-adamantane-1-carbonyl)-1,2,3,4-tetrahydroisoquinolin-7-yl)-3-(1-ethyl-4-methyl-1H-benzo[d][1,2,3]triazol-5-yl)propanoic acid; (146.6 mg, yield: 59%) as a white solid. ESI-MS (M+H)⁺: 527.7. ¹H NMR (400 MHz, CDCl₃) δ: 7.32-7.51 (m, 2H), 6.76-7.17 (m, 3H), 4.96 (t, J=7.91 Hz, 1H), 4.36-4.84 (m, 4H), 3.92 (d, J=5.77 Hz, 2H), 3.01-3.36 (m, 2H), 2.48-2.95 (m, 5H), 1.90-2.26 (m, 9H), 1.74 (br. s., 6H), 1.63 (t, J=7.28 Hz, 3H).

### Example 37

### 3-(2-(bicyclo[2.2.2]octane-1-carbonyl)-1,2,3,4-tetrahydroisoquinolin-7-yl)-3-(1-ethyl-4-methyl-1H-benzo[d][1,2,3]triazol-5-yl)propanoic acid;

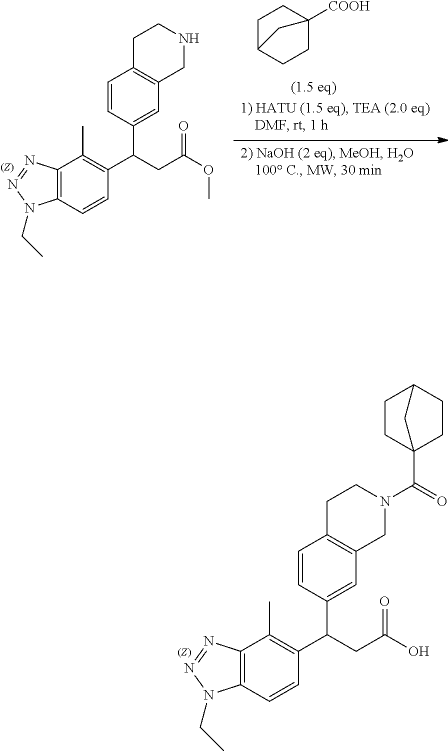

Synthesis of 3-(2-(bicyclo[2.2.2]octane-1-carbonyl)-1,2,3,4-tetrahydroisoquinolin-7-yl)-3-(1-ethyl-4-methyl-1H-benzo[d][1,2,3]triazol-5-yl)propanoic acid; was similar to that of Example 25. The residue was purified by prep-HPLC (CH₃CN/water with 0.1% CF₃COOH as mobile phase) to give 3-(2-(bicyclo[2.2.2]octane-1-carbonyl)-1,2,3,4-tetrahydroisoquinolin-7-yl)-3-(1-ethyl-4-methyl-1H-benzo[d][1,2,3]triazol-5-yl)propanoic acid; (101 mg, yield: 68%) as a white solid. ESI-MS (M+H)⁺: 501.3. ¹H NMR (400 MHz, CDCl₃) δ: 7.37 (s, 2H), 7.04 (s, 2H), 6.95 (s, 1H), 4.97 (t, J=7.91 Hz, 1H), 4.54-4.79 (m, 4H), 3.86 (br. s., 2H), 3.00-3.29 (m, 2H), 2.73-2.92 (m, 5H), 1.80-1.94 (m, 6H), 1.49-1.75 (m, 10H).

### Example 38

### 3-(1-ethyl-4-methyl-1H-benzo[d][1,2,3]triazol-5-yl)-3-(2-(cis-4-(trifluoromethyl)cyclohexane-1-carbonyl)-1,2,3,4-tetrahydroisoquinolin-7-yl)propanoic acid;

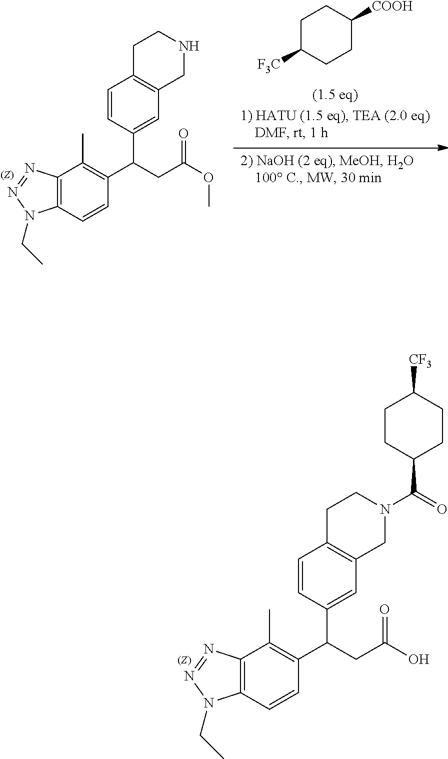

Synthesis of 3-(1-ethyl-4-methyl-1H-benzo[d][1,2,3]triazol-5-yl)-3-(2-(cis-4-(trifluoromethyl)cyclohexane-1-carbonyl)-1,2,3,4-tetrahydroisoquinolin-7-yl)propanoic acid; was similar to that of Example 25. The residue was purified by prep-HPLC (CH₃CN/water with 0.1% CF₃COOH as mobile phase) to give 3-(1-ethyl-4-methyl-1H-benzo[d][1,2,3]triazol-5-yl)-3-(2-(cis-4-(trifluoromethyl)cyclohexane-1-carbonyl)-1,2,3,4-tetrahydroisoquinolin-7-yl)propanoic acid; (147.8 mg, yield: 58%) as a white solid. ESI-MS (M+H)⁺: 543.2. ¹H NMR (400 MHz, CDCl₃) δ: 7.36 (d, J=7.03 Hz, 2H), 6.82-7.19 (m, 3H), 4.89-5.05 (m, 1H), 4.47-4.77 (m, 4H), 3.59-3.93 (m, 2H), 3.00-3.31 (m, 2H), 2.66-2.93 (m, 6H), 1.66-2.26 (m, 7H), 1.62 (d, J=14.56 Hz, 5H).

### Example 39

### 3-(1-ethyl-4-methyl-1H-benzo[d][1,2,3]triazol-5-yl)-3-(2-(trans-4-(trifluoromethyl)cyclohexane-1-carbonyl)-1,2,3,4-tetrahydroisoquinolin-7-yl)propanoic acid;

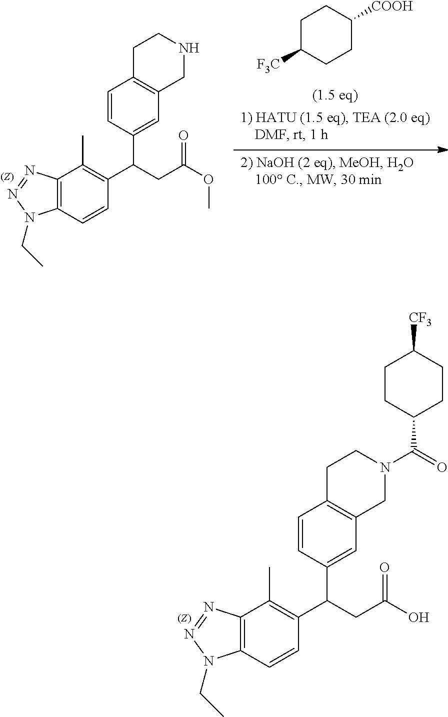

Synthesis of 3-(1-ethyl-4-methyl-1H-benzo[d][1,2,3]triazol-5-yl)-3-(2-(trans-4-(trifluoromethyl)cyclohexane-1-carbonyl)-1,2,3,4-tetrahydroisoquinolin-7-yl)propanoic acid; was similar to that of Example 25. The residue was purified by prep-HPLC (CH₃CN/water with 0.1% CF₃COOH as mobile phase) to give 3-(1-ethyl-4-methyl-1H-benzo[d][1,2,3]triazol-5-yl)-3-(2-(trans-4-(trifluoromethyl)cyclohexane-1-carbonyl)-1,2,3,4-tetrahydroisoquinolin-7-yl)propanoic acid; (162.5 mg, yield: 63%) as a white solid. ESI-MS (M+H)⁺: 543.2. ¹H NMR (400 MHz, CDCl₃) δ: 7.36 (br. s., 2H), 6.86-7.19 (m, 3H), 4.91-5.09 (m, 1H), 4.45-4.77 (m, 4H), 3.60-3.92 (m, 2H), 3.01-3.32 (m, 2H), 2.71-2.95 (m, 5H), 2.56 (t, J=11.55 Hz, 1H), 2.05 (d, J=12.05 Hz, 2H), 1.86 (br. s., 2H), 1.62 (t, J=7.28 Hz, 5H), 1.11-1.47 (m, 2H).

### Example 40

### 3-(1-ethyl-4-methyl-1H-benzo[d][1,2,3]triazol-5-yl)-3-(2-(2-phenylacetyl)-1,2,3,4-tetrahydroisoquinolin-7-yl)propanoic acid;

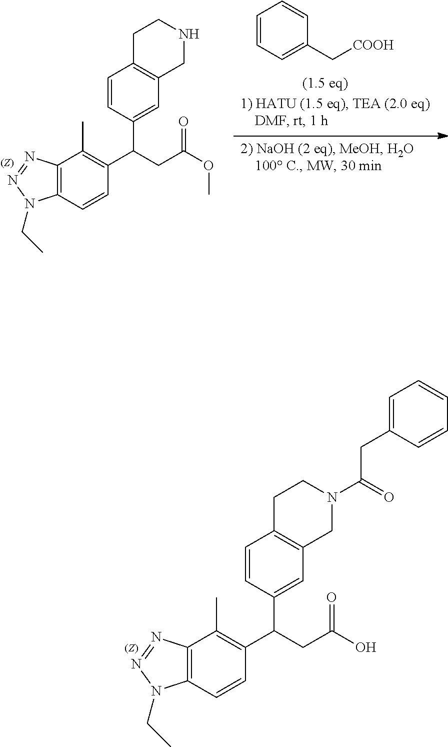

Synthesis of 3-(1-ethyl-4-methyl-1H-benzo[d][1,2,3]triazol-5-yl)-3-(2-(2-phenylacetyl)-1,2,3,4-tetrahydroisoquinolin-7-yl)propanoic acid; was similar to that of Example 25. The residue was purified by prep-HPLC (CH₃CN/water with 0.1% CF₃COOH as mobile phase) to give 3-(1-ethyl-4-methyl-1H-benzo[d][1,2,3]triazol-5-yl)-3-(2-(2-phenylacetyl)-1,2,3,4-tetrahydroisoquinolin-7-yl)propanoic acid; (57 mg, yield: 61%) as a white solid. ESI-MS (M+H)⁺: 483.2. ¹H NMR (400 MHz, CDCl₃) δ: 7.29-7.66 (m, 4H), 6.64-7.25 (m, 6H), 4.86-5.07 (m, 1H), 4.71 (s, 4H), 4.53 (s, 1H), 3.84 (s, 3H), 3.65 (t, J=5.77 Hz, 1H), 2.94-3.41 (m, 2H), 2.82 (s, 4H), 2.61 (br. s., 1H), 1.63 (t, J=7.28 Hz, 3H).

### Example 41

### (S)-3-(1-ethyl-4-methyl-1H-benzo[d][1,2,3]triazol-5-yl)-3-(2-(2-phenylacetyl)-1,2,3,4-tetrahydroisoquinolin-7-yl)propanoic acid; and (R)-3-(1-ethyl-4-methyl-1H-benzo[d][1,2,3]triazol-5-yl)-3-(2-(2-phenylacetyl)-1,2,3,4-tetrahydroisoquinolin-7-yl)propanoic acid;

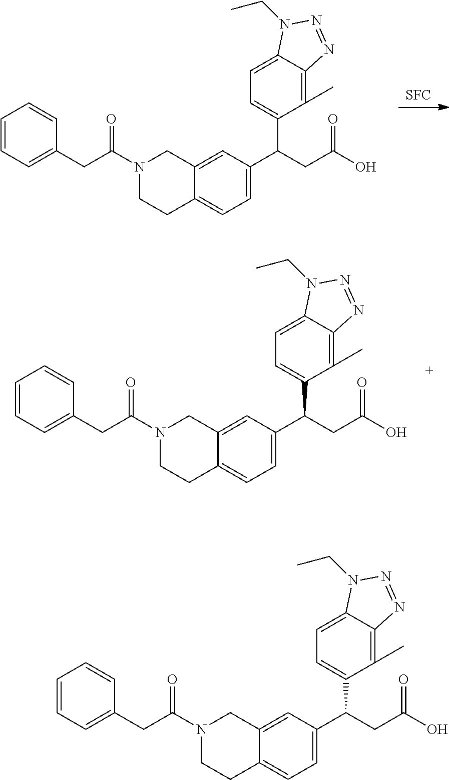 [SMILES: CCn1nnc2c(C)c(C(CC(=O)O)c3ccc4c(c3)CN(C(=O)Cc3ccccc3)CC4)ccc21 ; "SFC" ; CCn1nnc2c(C)c([C@@H](CC(=O)O)c3ccc4c(c3)CN(C(=O)Cc3ccccc3)CC4)ccc21 ; CCn1nnc2c(C)c([C@H](CC(=O)O)c3ccc4c(c3)CN(C(=O)Cc3ccccc3)CC4)ccc21]

3-(1-Ethyl-4-methyl-1H-benzo[d][1,2,3]triazol-5-yl)-3-(2-(2-phenylacetyl)-1,2,3,4-tetrahydroisoquinolin-7-yl)propanoic acid; (45.00 mg, 93.25 umol) was separated with the following condition: (Column: CHIRALPAK AS-H 30×250 mm, 5 um; Co-solvent: 35% Methanol w/0.1% DEA in CO2 (flow rate: 100 mL/min), ABPR 120 bar, MBPR 40 psi) to give peak 1 (9.4 mg, 100% ee, 100% purify) and peak 2 (8.3 mg, 100% ee). Both was further purified with prep HPLC (ACN/water 0.1% TFA) to give peak 1 (Ent-1) (7.50 mg, 14.76 umol, 15.83% yield, 95% purity). LCMS: RT=1.36 min, m/z=483.1; and peak 2 (Ent-2) (7.00 mg, 13.78 umol, 14.78% yield, 95% purity). LCMS: RT=1.36 min, m/z=483.1.

### Example 42

### 3-(1-ethyl-4-methyl-1H-benzo[d][1,2,3]triazol-5-yl)-3-(2-((S)-2-methoxy-2-phenylacetyl)-1,2,3,4-tetrahydroisoquinolin-7-yl)propanoic acid;

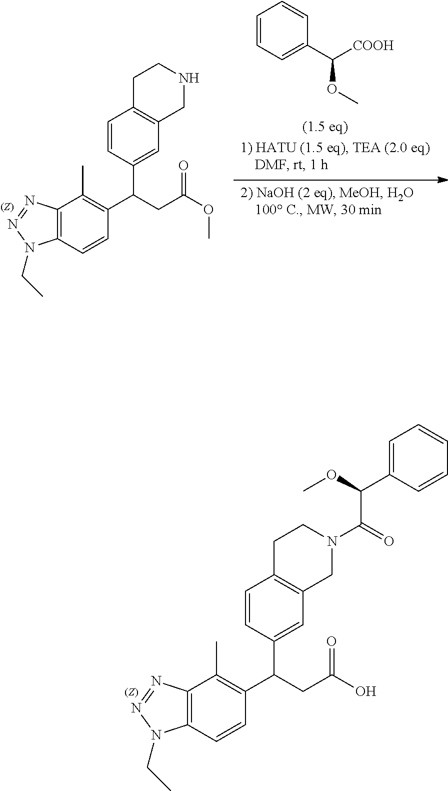

Synthesis of 3-(1-ethyl-4-methyl-1H-benzo[d][1,2,3]triazol-5-yl)-3-(2-((S)-2-methoxy-2-phenylacetyl)-1,2,3,4-tetrahydroisoquinolin-7-yl)propanoic acid; was similar to that of Example 25. The residue was purified by prep-HPLC (CH₃CN/water with 0.1% CF₃COOH as mobile phase) to give 3-(1-ethyl-4-methyl-1H-benzo[d][1,2,3]triazol-5-yl)-3-(2-((S)-2-methoxy-2-phenylacetyl)-1,2,3,4-tetrahydroisoquinolin-7-yl)propanoic acid; (131.6 mg, yield: 62%) as a white solid. ESI-MS (M+H)⁺: 513.2. ¹H NMR (400 MHz, CDCl₃) δ: 7.18-7.51 (m, 8H), 6.40-7.06 (m, 3H), 5.09 (s, 1H), 4.83-4.98 (m, 1H), 4.39-4.81 (m, 4H), 3.51-3.80 (m, 1H), 3.42 (s, 3H), 3.12 (br. s., 4H), 2.80 (s, 3H), 2.47 (br. s., 1H), 1.61 (t, J=7.34 Hz, 3H).

### Example 43

### 3-(1-ethyl-4-methyl-benzotriazol-5-yl)-3-[2-(piperidine-1-carbonyl)-3,4-dihydro-1H-isoquinolin-7-yl]propanoic acid;

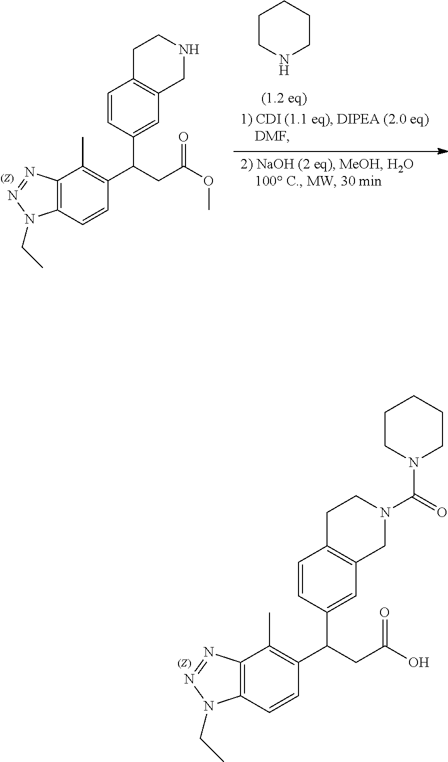

CDI (85.97 mg, 530.21 umol) in DMF (2.00 mL) was added DIPEA (74.75 mg, 578.41 umol, 101.02 uL) followed by methyl 3-(1-ethyl-4-methyl-benzotriazol-5-yl)-3-(1,2,3,4-tetrahydroisoquinolin-7-yl)propanoate (200.00 mg, 482.01 umol, Hydrochloride) and stirred at rt for 1 h. Then Piperidine (49.25 mg, 578.41 umol, 57.27 uL) and DIPEA (124.59 mg, 964.02 umol, 168.36 uL) was added and stirred overnight, then warmed up to 80° C. and stirred overnight. After quenching with MeOH, the crude was purified with prep HPLC to give methyl 3-(1-ethyl-4-methyl-benzotriazol-5-yl)-3-[2-(piperidine-1-carbonyl)-3,4-dihydro-1H-isoquinolin-7-yl]propanoate (113.00 mg, 230.80 umol, 47.88% yield). LCMS: Rt=1.61 min, m/z=490.2.

Methyl 3-(1-ethyl-4-methyl-benzotriazol-5-yl)-3-[2-(piperidine-1-carbonyl)-3,4-dihydro-1H-isoquinolin-7-yl]propanoate (113.00 mg, 230.80 umol) in methanol (2.00 mL) was added sodium hydroxide (2 M, 230.80 uL) and microwaved at 100° C. for 30 min. After neutralization with 2N HCl, the crude was purified with prep HPLC to give 3-(1-ethyl-4-methyl-benzotriazol-5-yl)-3-[2-(piperidine-1-carbonyl)-3,4-dihydro-1H-isoquinolin-7-yl]propanoic acid; (77.50 mg, 154.81 umol, 67.08% yield). ESI-MS (M+H)⁺: 476.2. ¹H NMR (400 MHz, CDCl₃) δ: 7.32-7.46 (m, 2H), 6.97-7.13 (m, 2H), 6.90 (s, 1H), 4.96 (t, J=7.78 Hz, 1H), 4.66 (q, J=7.28 Hz, 2H), 4.25-4.45 (m, 2H), 3.47 (t, J=5.65 Hz, 2H), 3.24 (br. s., 4H), 2.94-3.21 (m, 2H), 2.74-2.91 (m, 5H), 1.43-1.78 (m, 9H).

### Example 44

### 3-(1-ethyl-4-methyl-1H-benzo[d][1,2,3]triazol-5-yl)-3-(2-(4-(trifluoromethyl)piperidine-1-carbonyl)-1,2,3,4-tetrahydroisoquinolin-7-yl)propanoic acid;

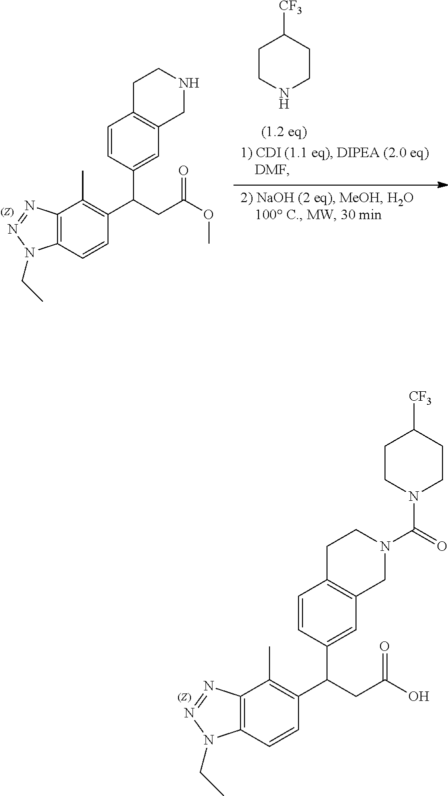

Synthesis of 3-(1-ethyl-4-methyl-1H-benzo[d][1,2,3]triazol-5-yl)-3-(2-(4-(trifluoromethyl)piperidine-1-carbonyl)-1,2,3,4-tetrahydroisoquinolin-7-yl)propanoic acid; was similar to that of Example 43. The residue was purified by prep-HPLC (CH₃CN/water with 0.1% CF₃COOH as mobile phase) to give 3-(1-ethyl-4-methyl-1H-benzo[d][1,2,3]triazol-5-yl)-3-(2-(4-(trifluoromethyl)piperidine-1-carbonyl)-1,2,3,4-tetrahydroisoquinolin-7-yl)propanoic acid; (20 mg, yield: 48%) as a white solid. ESI-MS (M+H)⁺: 544.3. ¹H NMR (400 MHz, CDCl₃) δ: 7.39 (s, 2H), 7.06 (s, 2H), 6.91 (s, 1H), 4.97 (t, J=7.91 Hz, 1H), 4.68 (q, J=7.28 Hz, 2H), 4.39 (s, 2H), 3.80 (d, J=13.05 Hz, 2H), 3.51 (t, J=5.77 Hz, 2H), 3.02-3.28 (m, 2H), 2.84-2.95 (m, 4H), 2.82 (s, 3H), 2.09-2.37 (m, 1H), 1.90 (d, J=12.55 Hz, 2H), 1.63 (t, J=7.28 Hz, 5H).

### Example 45

### 3-(1-ethyl-4-methyl-1H-benzo[d][1,2,3]triazol-5-yl)-3-(2-(methyl(phenyl)carbamoyl)-1,2,3,4-tetrahydroisoquinolin-7-yl)propanoic acid;

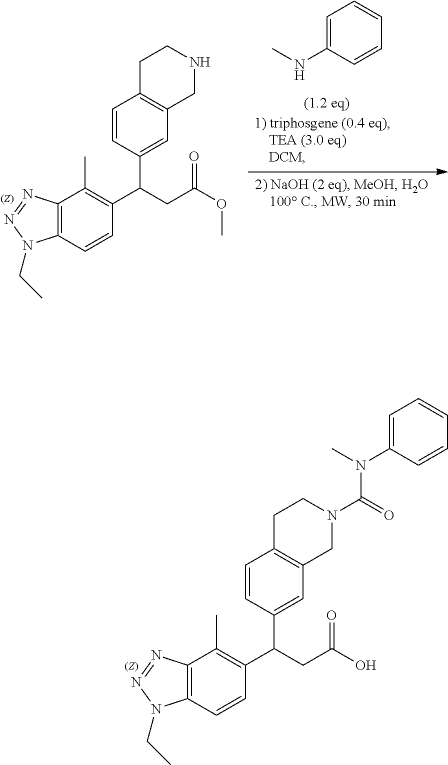

Synthesis of 3-(1-ethyl-4-methyl-1H-benzo[d][1,2,3]triazol-5-yl)-3-(2-(methyl(phenyl)carbamoyl-1,2,3,4-tetrahydroisoquinolin-7-yl)propanoic acid; was similar to that of Example 43. The residue was purified by prep-HPLC (CH₃CN/water with 0.1% CF₃COOH as mobile phase) to give 3-(1-ethyl-4-methyl-1H-benzo[d][1,2,3]triazol-5-yl)-3-(2-(4-(trifluoromethyl)piperidine-1-carbonyl)-1,2,3,4-tetrahydroisoquinolin-7-yl)propanoic acid; (149 mg, yield: 59%) as a white solid. ESI-MS (M+H)⁺: 498.3. ¹H NMR (400 MHz, CDCl₃) δ: 7.28-7.42 (m, 4H), 6.86-7.20 (m, 6H), 6.76 (s, 1H), 4.90 (t, J=7.78 Hz, 1H), 4.65 (q, J=7.28 Hz, 2H), 4.24 (s, 2H), 3.39 (t, J=5.77 Hz, 2H), 3.23 (s, 3H), 2.97-3.18 (m, 2H), 2.79 (s, 3H), 2.52 (t, J=5.52 Hz, 2H), 1.60 (t, J=7.40 Hz, 3H).

### Example 46

### 3-(1-ethyl-4-methyl-1H-benzo[d][1,2,3]triazol-5-yl)-3-(2-(2-isopropylmorpholine-4-carbonyl)-1,2,3,4-tetrahydroisoquinolin-7-yl)propanoic acid;

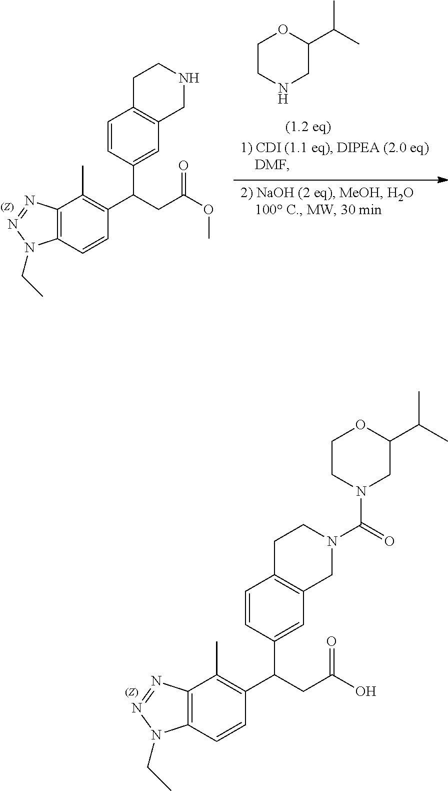

Synthesis of 3-(1-ethyl-4-methyl-1H-benzo[d][1,2,3]triazol-5-yl)-3-(2-(2-isopropylmorpholine-4-carbonyl)-1,2,3,4-tetrahydroisoquinolin-7-yl)propanoic acid; was similar to that of Example 43. The residue was purified by prep-HPLC (CH₃CN/water with 0.1% CF₃COOH as mobile phase) to give 3-(1-ethyl-4-methyl-1H-benzo[d][1,2,3]triazol-5-yl)-3-(2-(2-isopropylmorpholine-4-carbonyl)-1,2,3,4-tetrahydroisoquinolin-7-yl)propanoic acid; (12.5 mg, yield: 41%) as a white solid. ESI-MS (M+H)⁺: 520.3. ¹H NMR (400 MHz, CDCl₃) δ: 7.32-7.58 (m, 2H), 6.75-7.18 (m, 3H), 4.97 (t, J=7.91 Hz, 1H), 4.57-4.78 (m, 2H), 4.38 (s, 2H), 2.95-4.10 (m, 10H), 2.85 (s, 5H), 2.63-2.78 (m, 1H), 1.45-1.84 (m, 4H), 0.97 (d, J=6.78 Hz, 3H), 0.91 (d, J=6.78 Hz, 3H).

### Example 47

### (S)-3-(1-ethyl-4-methyl-1H-benzo[d][1,2,3]triazol-5-yl)-3-(2-(2,3,5,6-tetramethylbenzoyl)-1,2,3,4-tetrahydroisoquinolin-7-yl)propanoic acid; and (R)-3-(1-ethyl-4-methyl-1H-benzo[d][1,2,3]triazol-5-yl)-3-(2-(2,3,5,6-tetramethylbenzoyl)-1,2,3,4-tetrahydroisoquinolin-7-yl)propanoic acid;

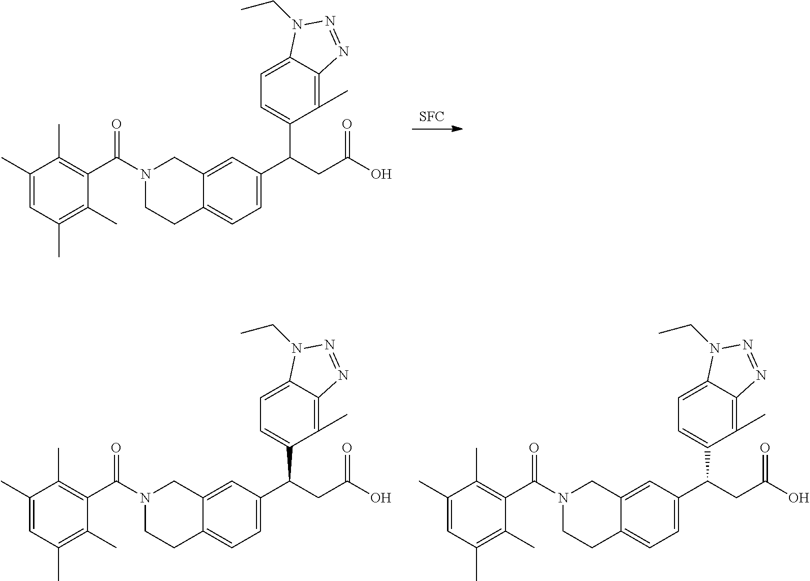

3-(1-Ethyl-4-methyl-1H-benzo[d][1,2,3]triazol-5-yl)-3-(2-(2,3,5,6-tetramethylbenzoyl)-1,2,3,4-tetrahydroisoquinolin-7-yl)propanoic acid; (195.00 mg, 371.68 umol) was separated under SFC condition: (Column: 2.1×25.0 cm Chiralcel OX-H from Chiral Tech; Solvent: CO₂/Ethanol with 025% Isopropylamine; Isocratic method: 53% Co-solvent at 75 g/min; system pressure: 110 bar; Column temperature 25° C.; sample diluent: ethanol.) to give peak D1 and D4 (atropisomers that interconvert upon standing) combined as (S)-3-(1-ethyl-4-methyl-1H-benzo[d][1,2,3]triazol-5-yl)-3-(2-(2,3,5,6-tetramethylbenzoyl)-1,2,3,4-tetrahydroisoquinolin-7-yl)propanoic acid; (38.60 mg, 72.25 umol, 19.44% yield, 98.2% purity), Ent-1. LCMS: Rt=1.48, 1.66 min, m/z=525.2. ¹H NMR (400 MHz, CDCl₃) δ: 7.30-7.50 (m, 2H), 6.62-7.19 (m, 4H), 3.85-5.16 (m, 7H), 2.60-3.51 (m, 7H), 2.13-2.30 (m, 6H), 1.91-2.10 (m, 6H), 1.49-1.72 (m, 3H). And peak D2 and D3 (atropisomers that interconvert upon standing) combined as (R)-3-(1-ethyl-4-methyl-1H-benzo[d][1,2,3]triazol-5-yl)-3-(2-(2,3,5,6-tetramethylbenzoyl)-1,2,3,4-tetrahydroisoquinolin-7-yl)propanoic acid; (47.00 mg, 86.00 umol, 23.14% yield, 96.0% purity), Ent-2. LCMS: Rt=1.48, 1.66 min, m/z=525.2. ¹H NMR (400 MHz, CDCl₃) δ: 7.30-7.50 (m, 2H), 6.62-7.19 (m, 4H), 3.85-5.16 (m, 7H), 2.60-3.51 (m, 7H), 2.13-2.30 (m, 6H), 1.91-2.10 (m, 6H), 1.49-1.72 (m, 3H).

### Example 48

### (S)-3-(1-ethyl-4-methyl-1H-benzo[d][1,2,3]triazol-5-yl)-3-(2-(2-methylbenzoyl)-1,2,3,4-tetrahydroisoquinolin-7-yl)propanoic acid; and (R)-3-(1-ethyl-4-methyl-1H-benzo[d][1,2,3]triazol-5-yl)-3-(2-(2-methylbenzoyl)-1,2,3,4-tetrahydroisoquinolin-7-yl)propanoic acid;

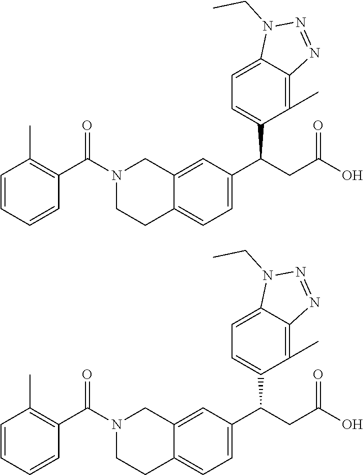

### 1. Preparation of 3-(1-ethyl-4-methyl-1H-benzo[d][1,2,3]triazol-5-yl)-3-(2-(2-methylbenzoyl)-1,2,3,4-tetrahydroisoquinolin-7-yl)propanoic acid;

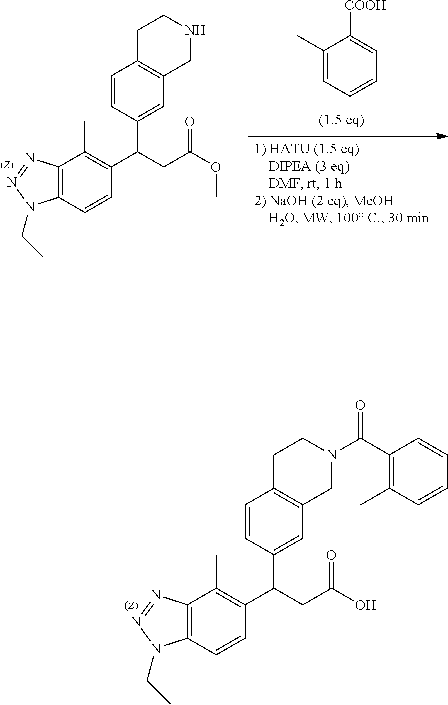

Synthesis of 3-(1-ethyl-4-methyl-1H-benzo[d][1,2,3]triazol-5-yl)-3-(2-(2-methylbenzoyl)-1,2,3,4-tetrahydroisoquinolin-7-yl)propanoic acid; was similar to that of Example 25. The residue was purified by prep-HPLC (MeCN/water with 0.05% HCOOH as mobile phase) to give 3-(1-ethyl-4-methyl-1H-benzo[d][1,2,3]triazol-5-yl)-3-(2-(2-methylbenzoyl)-1,2,3,4-tetrahydroisoquinolin-7-yl)propanoic acid; as white solid (240 mg, yield: 98%). ESI-MS (M+H)⁺: 483.2.

### 2. Preparation of (3S)-3-(1-ethyl-4-methyl-benzotriazol-5-yl)-3-[2-(2-methylbenzoyl)-3,4-dihydro-1H-isoquinolin-7-yl]propanoic acid; and (3R)-3-(1-ethyl-4-methyl-benzotriazol-5-yl)-3-[2-(2-methylbenzoyl)-3,4-dihydro-1H-isoquinolin-7-yl]propanoic acid;

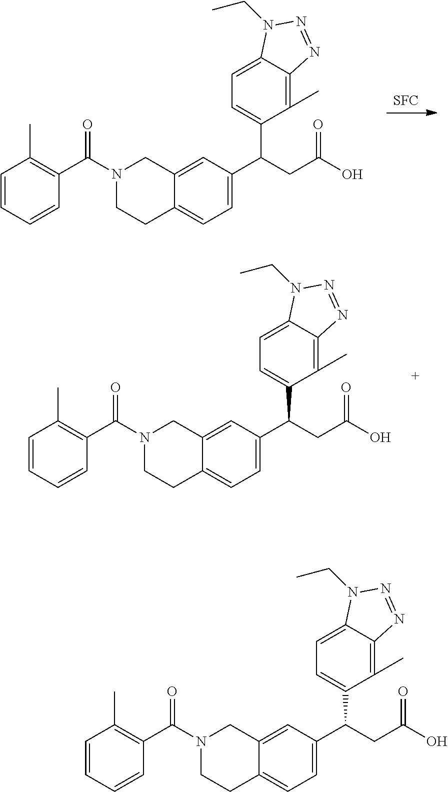 [SMILES: CCn1nnc2c(C)c(C(CC(=O)O)c3ccc4c(c3)CN(C(=O)c3ccccc3C)CC4)ccc21 ; "SFC" ; CCn1nnc2c(C)c([C@@H](CC(=O)O)c3ccc4c(c3)CN(C(=O)c3ccccc3C)CC4)ccc21 ; CCn1nnc2c(C)c([C@H](CC(=O)O)c3ccc4c(c3)CN(C(=O)c3ccccc3C)CC4)ccc21]

3-(1-Ethyl-4-methyl-benzotriazol-5-yl)-3-[2-(2-methylbenzoyl)-3,4-dihydro-1H-isoquinolin-7-yl]propanoic acid; (240.00 mg, 497.34 umol) was separated with the following SFC condition (Column: CHIRALPAK AS-H 30×250 mm, 5 um; Co-solvent: 25% Methanol w/0.1% DEA in CO2 (flow rate: 100 mL/min), ABPR 120 bar, MBPR 40 psi) to give the first eluate as Ent-1 (66.20 mg, 137.18 umol, 27.58% yield) and the second eluate as Ent-2 (69.20 mg, 143.40 umol, 28.83% yield). Ent 1: LCMS: rt=1.38 min, m/z=483.2. ¹H NMR (400 MHz, CDCl₃) δ: 6.59-7.61 (m, 9H), 2.90-5.18 (m, 10H), 2.79 (d, J=17.82 Hz, 4H), 2.09-2.35 (m, 3H), 1.48-1.75 (m, 3H). Ent 2: LCMS: rt=1.38 min, m/z=483.2. ¹H NMR (400 MHz, CDCl₃) δ: 6.59-7.61 (m, 9H), 2.90-5.18 (m, 10H), 2.79 (d, J=17.82 Hz, 4H), 2.09-2.35 (m, 3H), 1.48-1.75 (m, 3H).

### Example 49

### (S)-3-(2-benzoyl-1,2,3,4-tetrahydroisoquinolin-7-yl)-3-(1-ethyl-4-methyl-1H-benzo[d][1,2,3]triazol-5-yl)propanoic acid; and (R)-3-(2-benzoyl-1,2,3,4-tetrahydroisoquinolin-7-yl)-3-(1-ethyl-4-methyl-1H-benzo[d][1,2,3]triazol-5-yl)propanoic acid;

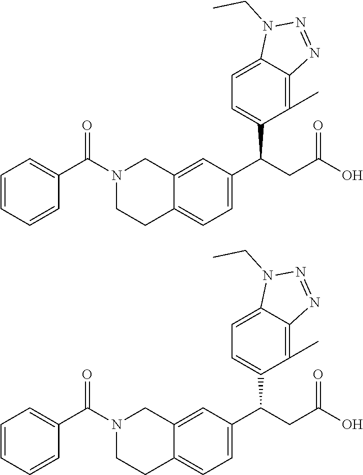

### 1. Preparation of 3-(2-benzoyl-1,2,3,4-tetrahydroisoquinolin-7-yl)-3-(1-ethyl-4-methyl-1H-benzo[d][1,2,3]triazol-5-yl)propanoic acid;

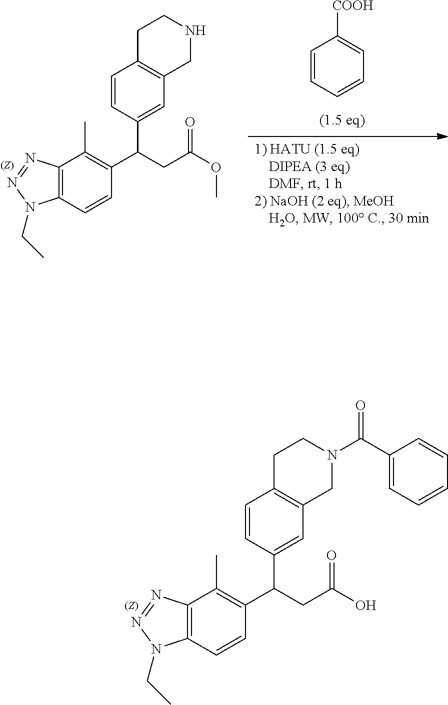

Synthesis of 3-(2-benzoyl-1,2,3,4-tetrahydroisoquinolin-7-yl)-3-(1-ethyl-4-methyl-1H-benzo[d][1,2,3]triazol-5-yl)propanoic acid; was similar to that of Example 25. The residue was purified by prep-HPLC (MeCN/water with 0.05% HCOOH as mobile phase) to give 3-(2-benzoyl-1,2,3,4-tetrahydroisoquinolin-7-yl)-3-(1-ethyl-4-methyl-1H-benzo[d][1,2,3]triazol-5-yl)propanoic acid; as white solid (320 mg, yield: 98%). ESI-MS (M+H)⁺: 469.2.

### 2. Preparation of (S)-3-(2-benzoyl-1,2,3,4-tetrahydroisoquinolin-7-yl)-3-(1-ethyl-4-methyl-1H-benzo[d][1,2,3]triazol-5-yl)propanoic acid; and (R)-3-(2-benzoyl-1,2,3,4-tetrahydroisoquinolin-7-yl)-3-(1-ethyl-4-methyl-1H-benzo[d][1,2,3]triazol-5-yl)propanoic acid;

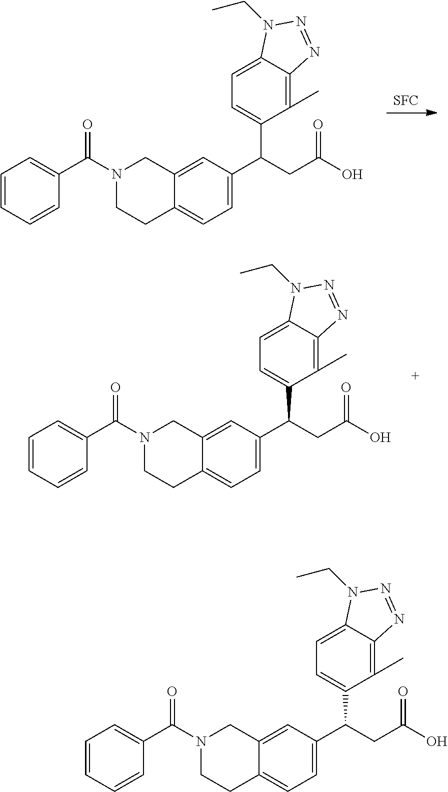 [SMILES: CCn1nnc2c(C)c(C(CC(=O)O)c3ccc4c(c3)CN(C(=O)c3ccccc3)CC4)ccc21 ; "SFC" ; CCn1nnc2c(C)c([C@@H](CC(=O)O)c3ccc4c(c3)CN(C(=O)c3ccccc3)CC4)ccc21 ; CCn1nnc2c(C)c([C@H](CC(=O)O)c3ccc4c(c3)CN(C(=O)c3ccccc3)CC4)ccc21]

3-(2-Benzoyl-3,4-dihydro-1H-isoquinolin-7-yl)-3-(1-ethyl-4-methyl-benzotriazol-5-yl)propanoic acid; (500.00 mg, 1.07 mmol) was purified with the following condition: (Column: CHIRALPAK AS-H 30×250 mm, 5 um; Co-solvent: 25% Methanol w/0.1% DEA in CO2 (flow rate: 100 mL/min), ABPR 120 bar, MBPR 40 psi) to give (3S)-3-(2-benzoyl-3,4-dihydro-1H-isoquinolin-7-yl)-3-(1-ethyl-4-methyl-benzotriazol-5-yl)propanoic acid; (75.00 mg, 160.07 umol, 15.00% yield) as peak 1 (Ent-1): LCMS: RT=1.34 min, m/z=469.2. ¹H NMR (400 MHz, CDCl₃) δ:7.42 (s, 7H), 7.07 (s, 3H), 4.24-5.26 (m, 4H), 2.35-4.09 (m, 10H), 1.60 (br. s., 3H). And (3R)-3-(2-benzoyl-3,4-dihydro-1H-isoquinolin-7-yl)-3-(1-ethyl-4-methyl-benzotriazol-5-yl)propanoic acid; (124.00 mg, 264.65 umol, 24.80% yield) as peak 2 (Ent-2). LCMS: RT=1.34 min, m/z=469.2. ¹H NMR (400 MHz, CDCl₃) δ:7.42 (s, 7H), 7.07 (s, 3H), 4.24-5.26 (m, 4H), 2.35-4.09 (m, 10H), 1.60 (br. s., 3H). The absolute configuration of peak1 was determined by an X-ray co-crystal structure with the KELCH domain of KEAP1.

### Example 50

### 3-[2-(benzenesulfonyl)-3,4-dihydro-1H-isoquinolin-7-yl]-3-(1-methylbenzotriazol-5-yl)propanoic acid;

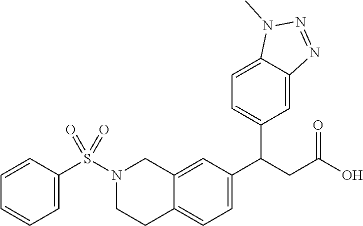 [SMILES: Cn1nnc2cc(C(CC(=O)O)c3ccc4c(c3)CN(S(=O)(=O)c3ccccc3)CC4)ccc21]

### 1. Preparation of (E)-3-(1-methylbenzotriazol-5-yl)prop-2-enoate

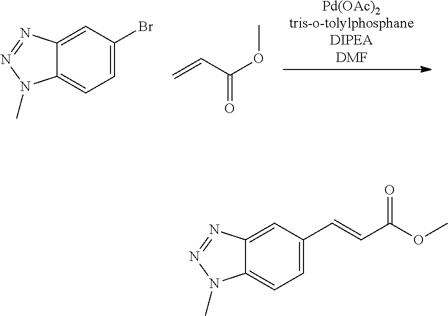

5-Bromo-1-methyl-benzotriazole (500.00 mg, 2.36 mmol), Pd(OAc)₂ (52.98 mg, 236.00 umol), tris-o-tolylphosphane (143.66 mg, 472.00 umol), DIPEA (915.02 mg,) in DMF (8.00 mL) was added methyl prop-2-enoate (2.03 g, 23.60 mmol, 2.12 mL), DIPEA (915.02 mg, 7.08 mmol, 1.24 mL) and degassed and microwaved at 130° C. for 2 h. After filtration through celite and diluted with EtOAc, the solution was washed with water and brine and dried over Na₂SO₄. Si gel chromatography gave methyl (E)-3-(1-methylbenzotriazol-5-yl)prop-2-enoate (240 mg, 47% yield). LCMS: Rt=1.04 min, m/z=218.

### 2. Preparation of tert-butyl 7-[3-methoxy-1-(1-methylbenzotriazol-5-yl)-3-oxo-propyl]-3,4-dihydro-1H-isoquinoline-2-carboxylate

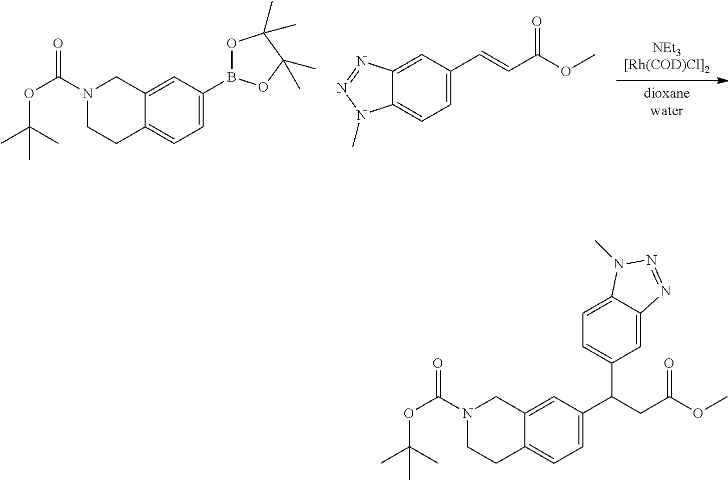

Tert-butyl 7-(4,4,5,5-tetramethyl-1,3,2-dioxaborolan-2-yl)-3,4-dihydro-1H-isoquinoline-2-carboxylate (148.86 mg, 414.33 umol), methyl (E)-3-(1-methylbenzotriazol-5-yl)prop-2-enoate (60.00 mg, 276.22 umol), N,N-diethylethanamine (41.93 mg, 414.33 umol, 57.43 uL) and [Rh(COD)Cl]₂ (6.81 mg, 13.81 umol) in dioxane (1.50 mL) and water (500.00 uL) was heated at 95° C. for overnight (19 h). After filtration, purification on prep HPLC gave tert-butyl 7-[3-methoxy-1-(1-methylbenzotriazol-5-yl)-3-oxo-propyl]-3,4-dihydro-1H-isoquinoline-2-carboxylate (16.50 mg, 36.62 umol, 13.26% yield). LCMS: Rt=1.66 min, m/z=451.2.

### 3. Preparation of methyl 3-(1-methyl-1H-benzo[d][1,2,3]triazol-5-yl)-3-(1,2,3,4-tetrahydroisoquinolin-7-yl)propanoate

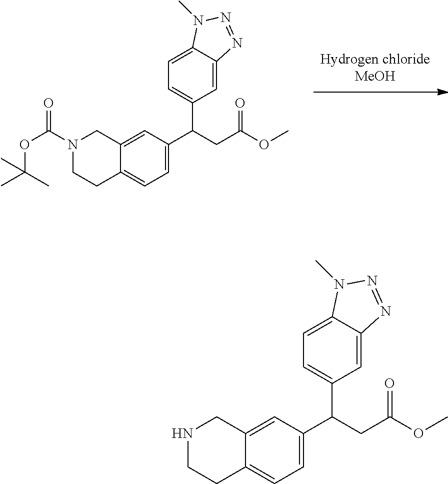 [SMILES: COC(=O)CC(c1ccc2c(c1)CN(C(=O)OC(C)(C)C)CC2)c1ccc2c(c1)nnn2C ; COC(=O)CC(c1ccc2c(c1)CNCC2)c1ccc2c(c1)nnn2C]

Tert-butyl 7-[3-methoxy-1-(1-methylbenzotriazol-5-yl)-3-oxo-propyl]-3,4-dihydro-1H-isoquinoline-2-carboxylate (342.00 mg, 759.11 umol) in MeOH (2.00 mL) was added Hydrogen chloride (4 M, 569.33 uL) in dioxane and stirred at rt for overnight. LCMS: Rt=0.80 min, m/z=351.1. After concentration, the crude was used for the next step directly.

### 4. Preparation of methyl 3-(1-methyl-1H-benzo[d][1,2,3]triazol-5-yl)-3-(2-(phenylsulfonyl)-1,2,3,4-tetrahydroisoquinolin-7-yl)propanoate

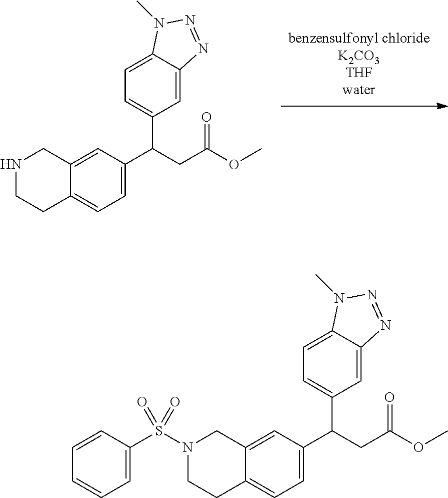 [SMILES: COC(=O)CC(c1ccc2c(c1)CNCC2)c1ccc2c(c1)nnn2C ; COC(=O)CC(c1ccc2c(c1)CN(S(=O)(=O)c1ccccc1)CC2)c1ccc2c(c1)nnn2C]

Methyl 3-(1-methylbenzotriazol-5-yl)-3-(1,2,3,4-tetrahydroisoquinolin-7-yl)propanoate (11.00 mg, 31.39 umol), K₂CO₃ (4.34 mg, 31.39 umol) in THF (2.00 mL) water (200.00 uL) was added benzenesulfonyl chloride (5.54 mg, 31.39 umol, 4.01 uL) and stirred at rt for overnight. After dilution with EtOAc and washing with water, brine and drying over Na₂SO₄, the concentrated residue was chromatographed on Si gel (HE/EA 0-100%) to give methyl 3-(1-methyl-1H-benzo[d][1,2,3]triazol-5-yl)-3-(2-(phenylsulfonyl)-1,2,3,4-tetrahydroisoquinolin-7-yl)propanoate (10.7 mg). LCMS: Rt=1.58 min, m/z=491.20. ¹H NMR (400 MHz, CHLOROFORM-d) δ 7.93 (s, 1H), 7.82 (d, J=7.28 Hz, 2H), 7.50-7.71 (m, 3H), 7.40-7.47 (m, 1H), 7.34 (d, J=8.53 Hz, 1H), 7.02 (d, J=13.05 Hz, 2H), 6.87 (s, 1H), 4.67 (t, J=7.78 Hz, 1H), 4.29 (s, 3H), 4.18 (s, 2H), 3.60 (s, 3H), 3.34 (s, 2H), 3.08 (s, 2H), 2.89 (t, J=5.77 Hz, 2H).

### 5. Preparation of 3-[2-(benzenesulfonyl)-3,4-dihydro-1H-isoquinolin-7-yl]-3-(1-methylbenzotriazol-5-yl)propanoic acid;

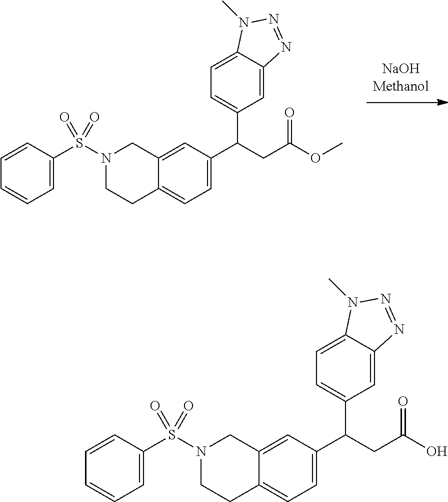 [SMILES: COC(=O)CC(c1ccc2c(c1)CN(S(=O)(=O)c1ccccc1)CC2)c1ccc2c(c1)nnn2C ; Cn1nnc2cc(C(CC(=O)O)c3ccc4c(c3)CN(S(=O)(=O)c3ccccc3)CC4)ccc21]

Methyl 3-[2-(benzenesulfonyl)-3,4-dihydro-1H-isoquinolin-7-yl]-3-(1-methylbenzotriazol-5-yl)propanoate (10.70 mg, 21.81 umol) in methanol was added sodium hydroxide (2M, 500.00 uL) and was microwaved at 100° C. for 30 min. After quenching with 1M HCl, the mixture was purified with prep HPLC to give 3-[2-(benzenesulfonyl)-3,4-dihydro-1H-isoquinolin-7-yl]-3-(1-methylbenzotriazol-5-yl)propanoic acid; (2.50 mg, 5.25 umol, 24.05% yield). LCMS: Rt=1.38 min, m/z=477.20.

### Example 51

### 3-(2-benzoyl-1,2,3,4-tetrahydroisoquinolin-7-yl)-3-(1-methyl-1H-benzo[d][1,2,3]triazol-5-yl)propanoic acid;

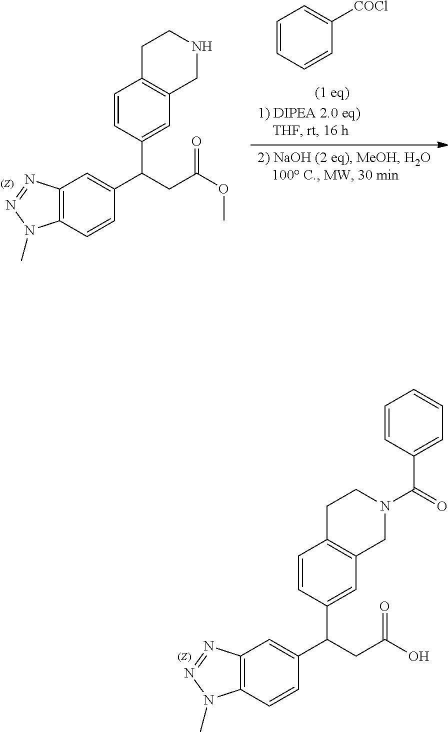

Preparation of methyl 3-(2-benzoyl-3,4-dihydro-1H-isoquinolin-7-yl)-3-(1-methylbenzotriazol-5-yl)propanoate was similar to that of Example 32. The residue was purified by prep-HPLC (CH₃CN/water with 0.1% CF₃COOH as mobile phase) to give 3-(2-benzoyl-3,4-dihydro-1H-isoquinolin-7-yl)-3-(1-methylbenzotriazol-5-yl)propanoic acid; (260.00 mg, 590.25 umol, 97.20% yield) LCMS: Rt=1.22 min, m/z=441.10.

### Example 52

### (S)-3-(2-benzoyl-1,2,3,4-tetrahydroisoquinolin-7-yl)-3-(1-methyl-1H-benzo[d][1,2,3]triazol-5-yl)propanoic acid; and (R)-3-(2-benzoyl-1,2,3,4-tetrahydroisoquinolin-7-yl)-3-(1-methyl-1H-benzo[d][1,2,3]triazol-5-yl)propanoic acid;

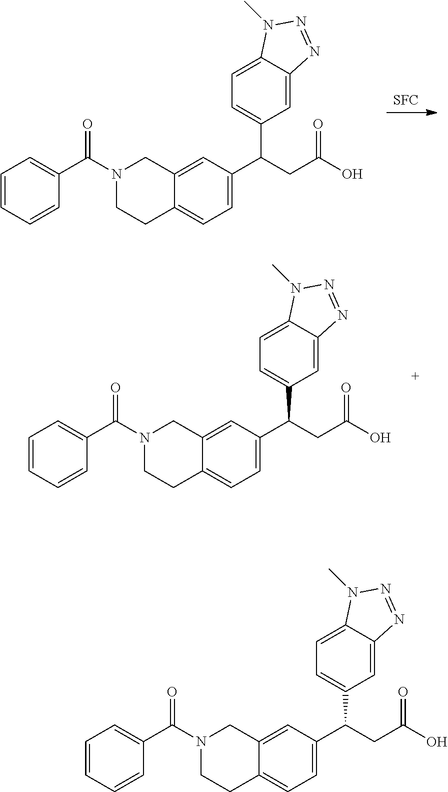 [SMILES: Cn1nnc2cc(C(CC(=O)O)c3ccc4c(c3)CN(C(=O)c3ccccc3)CC4)ccc21 ; "SFC" ; Cn1nnc2cc([C@@H](CC(=O)O)c3ccc4c(c3)CN(C(=O)c3ccccc3)CC4)ccc21 ; Cn1nnc2cc([C@H](CC(=O)O)c3ccc4c(c3)CN(C(=O)c3ccccc3)CC4)ccc21]

3-(2-Benzoyl-1,2,3,4-tetrahydroisoquinolin-7-yl)-3-(1-methyl-1H-benzo[d][1,2,3]triazol-5-yl)propanoic acid; (260.00 mg, 590.25 umol) was separated by SFC condition (Column: CHIRALPAK AS-H 30×250 mm, 5 um; Co-solvent: 30% Methanol w/0.1% DEA in CO2 (flow rate: 100 mL/min), ABPR 120 bar, MBPR 40 psi) to give the first eluate as Ent-1 (48.40 mg, 109.88 umol, 18.62% yield, 100% purity), and the second eluate as Ent-2 (53.20 mg, 120.77 umol, 20.60% yield, 100% purity). Peak1 (Ent-1): LCMS: Rt=1.22 min, m/z=441.1. ¹H NMR (400 MHz, CHLOROFORM-d) δ 7.94 (br. s., 1H), 7.42 (br. s., 7H), 6.63-7.18 (m, 3H), 4.41-5.12 (m, 4H), 4.24 (br. s., 3H), 3.93 (br. s., 1H), 3.59 (br. s., 1H), 2.71-3.17 (m, 4H). Peak 2 (Ent-2): LCMS: Rt=1.22 min, m/z=441.21. ¹H NMR (400 MHz, CHLOROFORM-d) δ 7.94 (br. s., 1H), 7.31-7.64 (m, 7H), 6.62-7.20 (m, 3H), 4.41-4.98 (m, 4H), 4.24 (br. s., 3H), 3.60 (br. s., 1H), 3.48 (br. s., 2H), 2.66-3.15 (m, 3H).

### Example 53

### (3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoic acid;

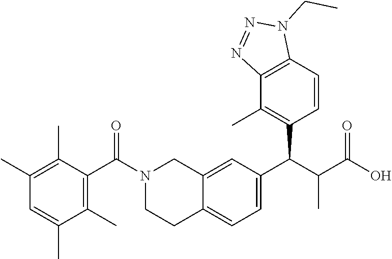 [SMILES: CCn1nnc2c(C)c([C@H](c3ccc4c(c3)CN(C(=O)c3c(C)c(C)cc(C)c3C)CC4)C(C)C(=O)O)ccc21]

### 1. Preparation of methyl (3S)-3-(1-ethyl-4-methyl-benzotriazol-5-yl)-3-(1,2,3,4-tetrahydroisoquinolin-7-yl)propanoate and methyl (3R)-3-(1-ethyl-4-methyl-benzotriazol-5-yl)-3-(1,2,3,4-tetrahydroisoquinolin-7-yl)propanoate

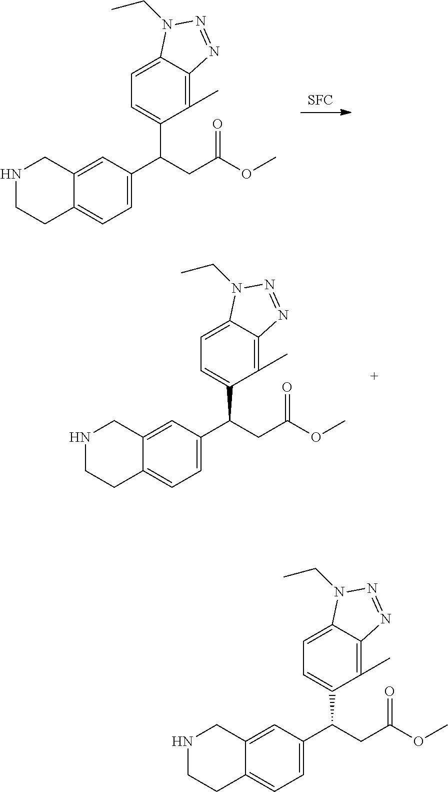 [SMILES: CCn1nnc2c(C)c(C(CC(=O)OC)c3ccc4c(c3)CNCC4)ccc21 ; "SFC" ; CCn1nnc2c(C)c([C@@H](CC(=O)OC)c3ccc4c(c3)CNCC4)ccc21 ; CCn1nnc2c(C)c([C@H](CC(=O)OC)c3ccc4c(c3)CNCC4)ccc21]

Methyl 3-(1-ethyl-4-methyl-benzotriazol-5-yl)-3-(1,2,3,4-tetrahydroisoquinolin-7-yl)propanoate (3.00 g, 7.23 mmol, Hydrochloride) was separated using the purification method (Column: CHIRALPAK AD-H 30×250 mm, 5 um; Co-solvent: 40% 2-Propanol in 0.1% DEA in CO₂ (flow rate: 100 mL/min), ABPR 120 bar, MBPR 60 psi.) to give methyl (3S)-3-(1-ethyl-4-methyl-benzotriazol-5-yl)-3-(1,2,3,4-tetrahydroisoquinolin-7-yl)propanoate (868.00 mg, 2.29 mmol, 31.72% yield), LCMS: Rt=0.91 min, m/z=379.2. And methyl (3R)-3-(1-ethyl-4-methyl-benzotriazol-5-yl)-3-(1,2,3,4-tetrahydroisoquinolin-7-yl)propanoate (843.00 mg, 2.23 mmol, 30.81% yield). LCMS: Rt=0.91 min, m/z=379.2.

### 2. Preparation of methyl (3S)-3-(1-ethyl-4-methyl-benzotriazol-5-yl)-3-[2-(2,3,5,6-tetramethylbenzoyl)-3,4-dihydro-1H-isoquinolin-7-yl]propanoate

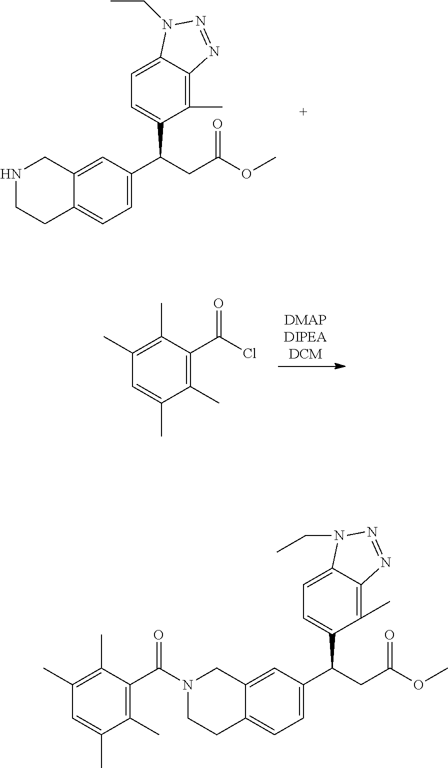

Methyl (3S)-3-(1-ethyl-4-methyl-benzotriazol-5-yl)-3-(1,2,3,4-tetrahydroisoquinolin-7-yl)propanoate (141.00 mg, 339.82 umol, Hydrochloride), 2,3,5,6-tetramethylbenzoyl chloride (73.52 mg, 373.80 umol), DMAP (4.15 mg, 33.98 umol) in DCM (2.00 mL) was added DIPEA (131.76 mg, 1.02 mmol, 178.05 uL) and stirred at rt for overnight. The concentrated residue was chromatographed on Si gel (HE/EA 0-100%) to give methyl (3S)-3-(1-ethyl-4-methyl-benzotriazol-5-yl)-3-[2-(2,3,5,6-tetramethylbenzoyl)-3,4-dihydro-1H-isoquinolin-7-yl]propanoate (130.00 mg, 241.33 umol, 71.02% yield). LCMS: Rt=1.71, 1.85 min, m/z=539.2.

### 3. Preparation of methyl (3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoate

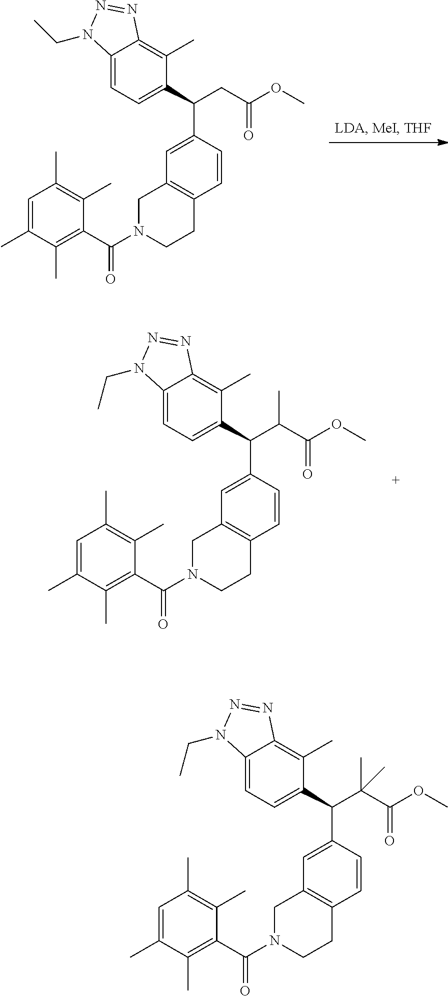

Methyl (3S)-3-(1-ethyl-4-methyl-benzotriazol-5-yl)-3-[2-(2,3,5,6-tetramethylbenzoyl)-3,4-dihydro-1H-isoquinolin-7-yl]propanoate (85.00 mg, 157.79 umol) in THF (2.00 mL) was added MeI (28.75 mg, 205.13 umol, 28.19 uL) and cooled to −78° C., then LDA (1 M, 394.48 uL) was added and warmed up to rt over 2 h. After quenching with water and extraction with EtOAc, the dried concentrated residue was chromatographed on Si gel (HE/EA 0-100%) to give methyl (3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoate (34.60 mg, 62.60 umol, 39.67% yield). LCMS: Rt=1.77, 1.93 min, m/z=553.3. And dimethyl side product methyl (S)-3-(1-ethyl-4-methyl-1H-benzo[d][1,2,3]triazol-5-yl)-2,2-dimethyl-3-(2-(2,3,5,6-tetramethylbenzoyl)-1,2,3,4-tetrahydroisoquinolin-7-yl)propanoate (37 mg, yield 40%). LCMS: Rt=1.98 min, m/z=567.3.

### 4. Preparation of (3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoic acid;

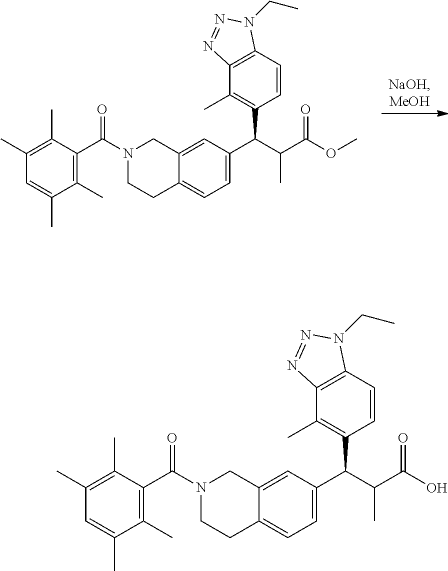

Methyl (3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoate (34.60 mg, 62.60 umol) in methanol (2.00 mL) was added sodium hydroxide (2 M, 62.60 uL) and microwaved at 100° C. for 30 min. After neutralizing with 2M HCl, the crude was purified with prep HPLC to give (3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoic acid; (10.00 mg, 17.64 umol, 28.17% yield, 95% purity). LCMS: Rt=1.52 min, m/z=539.3. ¹H NMR (400 MHz, CHLOROFORM-d) δ 7.29-7.69 (m, 2H), 6.63-7.24 (m, 4H), 3.77-5.00 (m, 6H), 2.90-3.57 (m, 2H), 2.62-2.89 (m, 3H), 1.87-2.33 (m, 13H), 1.48-1.74 (m, 3H), 0.99-1.39 (m, 3H). NMR shows a 2:1 mixture.

### Example 54

### (3S)-3-(1-ethyl-4-methyl-benzotriazol-5-yl)-2,2-dimethyl-3-[2-(2,3,5,6-tetramethylbenzoyl)-3,4-dihydro-1H-isoquinolin-7-yl]propanoic acid;

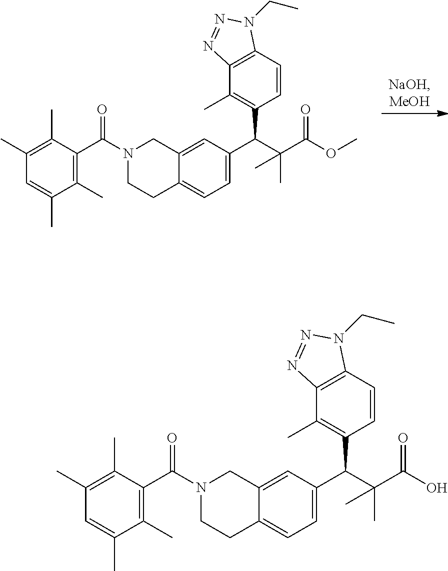

Methyl (3S)-3-(1-ethyl-4-methyl-benzotriazol-5-yl)-2,2-dimethyl-3-[2-(2,3,5,6-tetramethylbenzoyl)-3,4-dihydro-1H-isoquinolin-7-yl]propanoate (37.00 mg, 65.29 umol) in methanol (2.00 mL) was added sodium hydroxide (2 M, 65.29 uL) and microwaved at 100° C. for 30 min. After neutralization with 2M HCl, the crude was purified with prep HPLC to give (3S)-3-(1-ethyl-4-methyl-benzotriazol-5-yl)-2,2-dimethyl-3-[2-(2,3,5,6-tetramethylbenzoyl)-3,4-dihydro-1H-isoquinolin-7-yl]propanoic acid; (3.80 mg, 6.53 umol, 20.01% yield, 95% purity). LCMS: Rt=1.69 min, m/z=553.2. ¹H NMR (400 MHz, CHLOROFORM-d) δ 7.34-7.66 (m, 2H), 6.77-7.25 (m, 4H), 3.22-6.19 (m, 6H), 2.96 (s, 3H), 2.47-2.89 (m, 2H), 1.94-2.37 (m, 13H), 1.47-1.76 (m, 6H), 1.18 (d, J=6.78 Hz, 3H). And its rotamer (3S)-3-(1-ethyl-4-methyl-benzotriazol-5-yl)-2,2-dimethyl-3-[2-(2,3,5,6-tetramethylbenzoyl)-3,4-dihydro-1H-isoquinolin-7-yl]propanoic acid; (5.30 mg, 9.11 umol, 27.91% yield, 95% purity) LCMS: Rt=1.78 min, m/z=553.2. ¹H NMR (400 MHz, CHLOROFORM-d) δ 7.30-7.82 (m, 2H), 6.77-7.21 (m, 4H), 3.13-6.22 (m, 6H), 2.84 (d, J=8.53 Hz, 3H), 2.46-2.78 (m, 2H), 1.96-2.33 (m, 13H), 1.48-1.83 (m, 6H), 1.29 (d, J=6.78 Hz, 3H).

### Example 55

### (R)-2-((S)-(1-ethyl-4-methyl-1H-benzo[d][1,2,3]triazol-5-yl)(2-(2,3,5,6-tetramethylbenzoyl)-1,2,3,4-tetrahydroisoquinolin-7-yl)methyl)butanoic acid; and (S)-2-((S)-(1-ethyl-4-methyl-1H-benzo[d][1,2,3]triazol-5-yl)(2-(2,3,5,6-tetramethylbenzoyl)-1,2,3,4-tetrahydroisoquinolin-7-yl)methyl)butanoic acid;

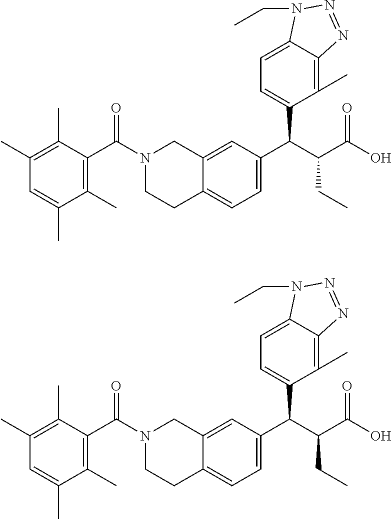 [SMILES: CC[C@@H](C(=O)O)[C@@H](c1ccc2c(c1)CN(C(=O)c1c(C)c(C)cc(C)c1C)CC2)c1ccc2c(nnn2CC)c1C ; CC[C@H](C(=O)O)[C@@H](c1ccc2c(c1)CN(C(=O)c1c(C)c(C)cc(C)c1C)CC2)c1ccc2c(nnn2CC)c1C]

### 1. Preparation of tert-butyl 7-[(1S)-1-(1-ethyl-4-methyl-benzotriazol-5-yl)-3-methoxy-3-oxo-propyl]-3,4-dihydro-1H-isoquinoline-2-carboxylate

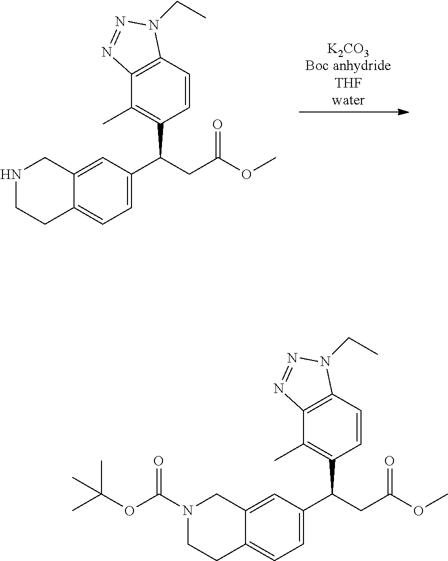

Methyl (3S)-3-(1-ethyl-4-methyl-benzotriazol-5-yl)-3-(1,2,3,4-tetrahydroisoquinolin-7-yl)propanoate (600.00 mg, 1.45 mmol, Hydrochloride), K₂CO₃ (599.57 mg, 4.34 mmol), Boc anhydride (473.39 mg, 2.17 mmol, 498.31 uL) in THF (2.00 mL) and water (2.00 mL) was stirred at rt for overnight. After dilution with EtOAc, the organic layer was washed with brine and died and chromatographed on Si gel (HE/EA 0-100%) to give tert-butyl 7-[(1S)-1-(1-ethyl-4-methyl-benzotriazol-5-yl)-3-methoxy-3-oxo-propyl]-3,4-dihydro-1H-isoquinoline-2-carboxylate (593.00 mg, 1.24 mmol, 85.75% yield). LCMS: Rt=1.80 min, m/z=479.2.

### 2. Preparation of tert-butyl 7-[(1S)-1-(1-ethyl-4-methyl-benzotriazol-5-yl)-2-methoxycarbonyl-butyl]-3,4-dihydro-1H-isoquinoline-2-carboxylate Isomer 1 and tert-butyl 7-[(1S)-1-(1-ethyl-4-methyl-benzotriazol-5-yl)-2-methoxycarbonyl-butyl]-3,4-dihydro-1H-isoquinoline-2-carboxylate Isomer 2

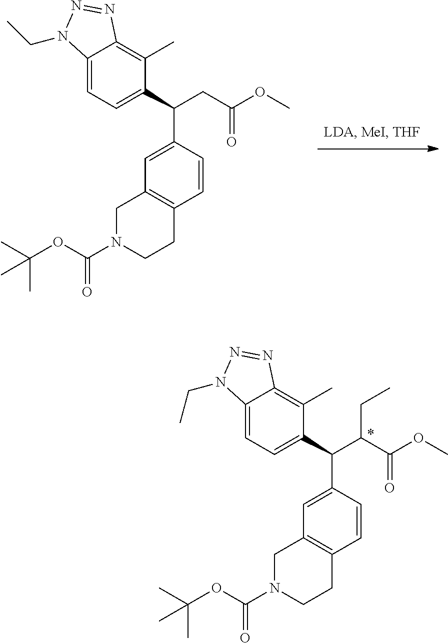

LDA (1 M, 31.23 uL) in THF (2.00 mL) was cooled to −78° C. and tert-butyl 7-[(1S)-1-(1-ethyl-4-methyl-benzotriazol-5-yl)-3-methoxy-3-oxo-propyl]-3,4-dihydro-1H-isoquinoline-2-carboxylate (100.00 mg, 208.95 umol) in THF (2.00 mL) was added and stirred for 2 h and warmed to −20° C., and Ethyl iodide (48.88 mg, 313.43 umol, 25.20 uL) was added and stirred overnight. After quenching with water, the mixture was extracted with EtOAc and dried and concentrated, the residue was chromatographed with Si gel (HE-EA 0-65%) to give peak 1 tert-butyl 7-[(1S)-1-(1-ethyl-4-methyl-benzotriazol-5-yl)-2-methoxycarbonyl-butyl]-3,4-dihydro-1H-isoquinoline-2-carboxylate Isomer 1 (30.10 mg, 59.41 umol, 28.43% yield). LCMS: Rt=1.90 min, m/z=507.3. And peak 2 tert-butyl 7-[(1S)-1-(1-ethyl-4-methyl-benzotriazol-5-yl)-2-methoxycarbonyl-butyl]-3,4-dihydro-1H-isoquinoline-2-carboxylate Isomer 2 (37.00 mg, 73.03 umol, 34.95% yield). LCMS: Rt=1.94 min, m/z=507.3.

### 3. Preparation of methyl 2-((S)-(1-ethyl-4-methyl-1H-benzo[d][1,2,3]triazol-5-yl)(1,2,3,4-tetrahydroisoquinolin-7-yl)methyl)butanoate Isomer 1

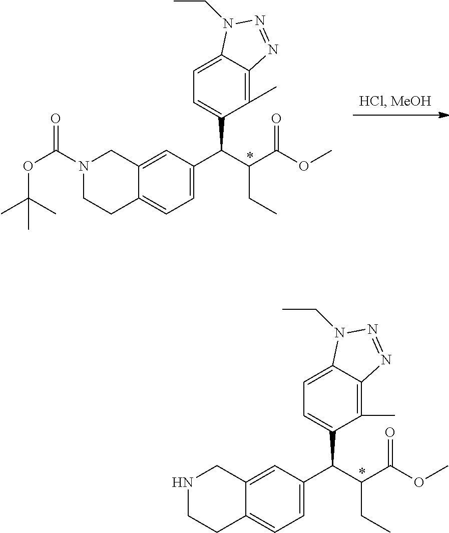

tert-butyl 7-((1S)-1-(1-ethyl-4-methyl-1H-benzo[d][1,2,3]triazol-5-yl)-2-(methoxycarbonyl)butyl)-3,4-dihydroisoquinoline-2(1H)-carboxylate Isomer 1 (30.60 mg, 60.40 umol) in MeOH (2.00 mL) was added HCl (4 M, 30.20 uL) and stirred at rt for overnight. The crude methyl 2-((S)-(1-ethyl-4-methyl-1H-benzo[d][1,2,3]triazol-5-yl)(1,2,3,4-tetrahydroisoquinolin-7-yl)methyl)butanoate Isomer 1 (27.00 mg, 60.95 umol, 100.91% yield, Hydrochloride) was concentrated down and used for the next step directly. LCMS: Rt=1.07 min, m/z=407.2.

### 4. Preparation methyl 2-((S)-(1-ethyl-4-methyl-1H-benzo[d][1,2,3]triazol-5-yl)(2-(2,3,5,6-tetramethylbenzoyl)-1,2,3,4-tetrahydroisoquinolin-7-yl)methyl)butanoate Isomer 1

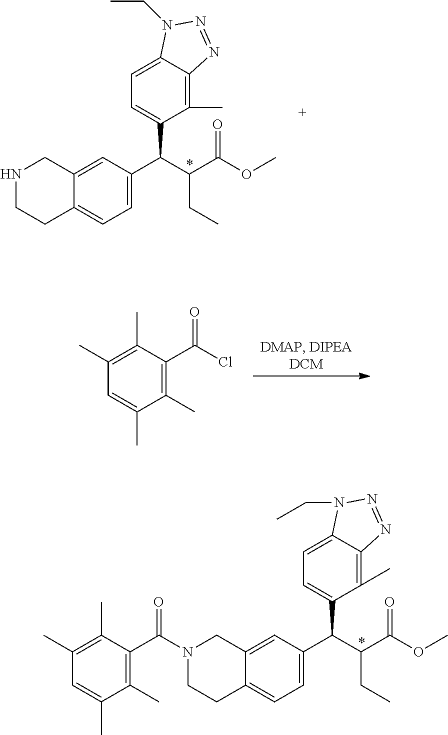

methyl 2-((S)-(1-ethyl-4-methyl-1H-benzo[d][1,2,3]triazol-5-yl)(1,2,3,4-tetrahydroisoquinolin-7-yl)methyl)butanoate Isomer 1 (27.00 mg, 60.95 umol, Hydrochloride), 2,3,5,6-tetramethylbenzoyl chloride (11.99 mg, 60.95 umol), DMAP (7.45 mg, 60.95 umol), DIPEA (23.63 mg, 182.85 umol, 31.93 uL) in DCM (2.00 mL) was heated to 40° C. for overnight. After cooling down, the concentrated residue was chromatographed on Si gel (HE/EA 0-100%) to give methyl 2-((S)-(1-ethyl-4-methyl-1H-benzo[d][1,2,3]triazol-5-yl)(2-(2,3,5,6-tetramethylbenzoyl)-1,2,3,4-tetrahydroisoquinolin-7-yl)methyl)butanoate Isomer 1 (20.40 mg, 36.00 umol, 59.06% yield). LCMS: Rt=1.87, 1.97 min, m/z=567.3.

### 5. Preparation of 2-((S)-(1-ethyl-4-methyl-1H-benzo[d][1,2,3]triazol-5-yl)(2-(2,3,5,6-tetramethylbenzoyl)-1,2,3,4-tetrahydroisoquinolin-7-yl)methyl)butanoic acid; Isomer 1

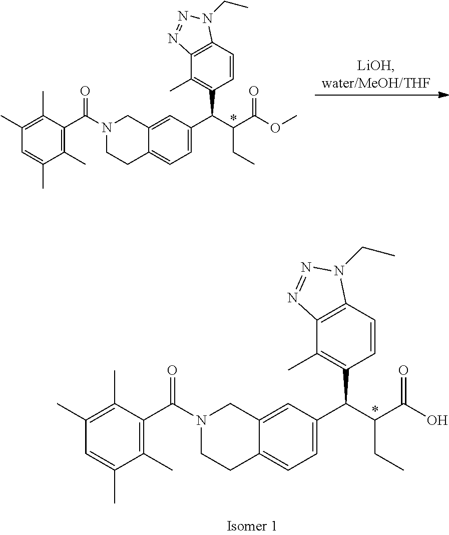

methyl 2-((S)-(1-ethyl-4-methyl-1H-benzo[d][1,2,3]triazol-5-yl)(2-(2,3,5,6-tetramethylbenzoyl)-1,2,3,4-tetrahydroisoquinolin-7-yl)methyl)butanoate Isomer 1 (20.40 mg, 36.00 umol) in Methanol (1.00 mL), THF (500.00 uL) and water (500.00 uL) was added Lithium hydroxide (2.59 mg, 108.00 umol) and microwaved at 100° C. for 1 h. After neutralization with 2M HCl, the crude was purified with prep HPLC to give Ent-1, 2-((S)-(1-ethyl-4-methyl-1H-benzo[d][1,2,3]triazol-5-yl)(2-(2,3,5,6-tetramethylbenzoyl)-1,2,3,4-tetrahydroisoquinolin-7-yl)methyl)butanoic acid; Isomer 1 (3.20 mg, 5.50 umol, 15.28% yield, 95% purity). LCMS: Rt=1.58, 1.70 min, m/z=553.2. ¹H NMR (400 MHz, CHLOROFORM-d) δ 7.31-7.75 (m, 2H), 6.65-7.23 (m, 4H), 2.40-5.00 (m, 13H), 1.86-2.29 (m, 12H), 1.61 (t, J=7.28 Hz, 5H), 0.72-1.12 (m, 3H).

### 6. Preparation of 2-((S)-(1-ethyl-4-methyl-1H-benzo[d][1,2,3]triazol-5-yl)(2-(2,3,5,6-tetramethylbenzoyl)-1,2,3,4-tetrahydroisoquinolin-7-yl)methyl)butanoic acid; Isomer 2

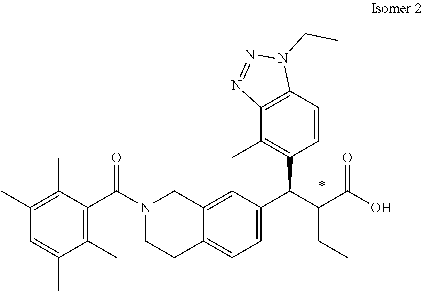 [SMILES: CCC(C(=O)O)[C@@H](c1ccc2c(c1)CN(C(=O)c1c(C)c(C)cc(C)c1C)CC2)c1ccc2c(nnn2CC)c1C]

2-((S)-(1-ethyl-4-methyl-1H-benzo[d][1,2,3]triazol-5-yl)(2-(2,3,5,6-tetramethylbenzoyl)-1,2,3,4-tetrahydroisoquinolin-7-yl)methyl)butanoic acid; Isomer 2 was made from tert-butyl 7-[(1S)-1-(1-ethyl-4-methyl-benzotriazol-5-yl)-2-methoxycarbonyl-butyl]-3,4-dihydro-1H-isoquinoline-2-carboxylate Isomer 2 following the procedures of 2-((S)-(1-ethyl-4-methyl-1H-benzo[d][1,2,3]triazol-5-yl)(2-(2,3,5,6-tetramethylbenzoyl)-1,2,3,4-tetrahydroisoquinolin-7-yl)methyl)butanoic acid; Isomer 1. After neutralization with 2M HCl, the crude was purified with prep. HPLC to give Ent-2, 2-((S)-(1-ethyl-4-methyl-1H-benzo[d][1,2,3]triazol-5-yl)(2-(2,3,5,6-tetramethylbenzoyl)-1,2,3,4-tetrahydroisoquinolin-7-yl)methyl)butanoic acid; Isomer 2 (6 mg, 19% yield). LCMS: Rt=1.58, 1.69 min, m/z=553.3. ¹H NMR (400 MHz, CHLOROFORM-d) δ 7.60 (d, J=8.53 Hz, 1H), 7.33 (d, J=8.78 Hz, 1H), 7.14 (d, J=7.28 Hz, 2H), 7.00 (s, 1H), 6.81 (s, 1H), 2.55-4.96 (m, 13H), 1.87-2.28 (m, 12H), 1.59 (t, J=7.28 Hz, 5H), 0.68-1.12 (m, 3H).

### Example 56

### 3-(6-methoxy-4-methyl-3-pyridyl)-3-[2-(2,3,5,6-tetramethylbenzoyl)-3,4-dihydro-1H-isoquinolin-7-yl]propanoic acid;

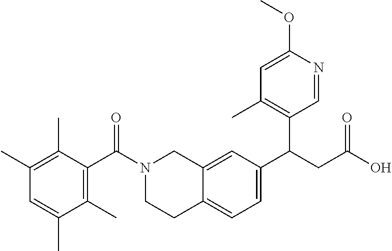 [SMILES: COc1cc(C)c(C(CC(=O)O)c2ccc3c(c2)CN(C(=O)c2c(C)c(C)cc(C)c2C)CC3)cn1]

### 1. Preparation of methyl (E)-3-(6-methoxy-4-methyl-3-pyridyl)prop-2-enoate

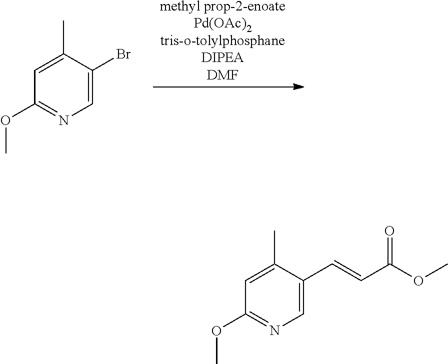

5-Bromo-2-methoxy-4-methyl-pyridine (500.00 mg, 2.47 mmol), methyl prop-2-enoate (1.06 g, 12.35 mmol, 1.11 mL), Pd(OAc)₂ (55.45 mg, 247.00 umol), tris-o-tolylphosphane (150.36 mg, 494.00 umol) and DIPEA (957.67 mg, 7.41 mmol, 1.29 mL) in DMF (5.00 mL) was microwaved at 120° C. for 2 h. After dilution with EtOAc and filtration, the organic layer was washed with water, brine and dried over Na₂SO₄. Chromatography on Si gel (DCM/MeOH 0-100%) gave methyl (E)-3-(6-methoxy-4-methyl-3-pyridyl)prop-2-enoate (504.00 mg, 2.19 mmol, 88.6% yield, 90% purity). LCMS: Rt=1.07 min, m/z=208.1.

### 2. Preparation of 7-bromo-3,4-dihydroisoquinolin-2(1H)-yl)(2,3,5,6-tetramethylphenyl)methanone

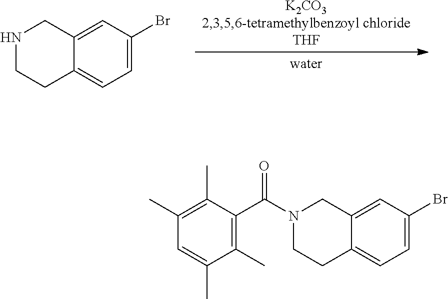 [SMILES: Brc1ccc2c(c1)CNCC2 ; "K2CO32,3,5,6-tetramethylbenzoyl chlorideTHF" ; "water" ; Cc1cc(C)c(C)c(C(=O)N2CCc3ccc(Br)cc3C2)c1C]

7-Bromo-1,2,3,4-tetrahydroisoquinoline (500.00 mg, 2.36 mmol), K₂CO₃ (978.53 mg, 7.08 mmol), 2,3,5,6-tetramethylbenzoyl chloride (464.14 mg, 2.36 mmol) in THF (5.00 mL) and water (499.95 uL) was stirred at rt for overnight. After dilution with EtOAc and washing with water and brine, the dried residue was purified by chromatograph on Si gel (HE/EA 0-100%) to give (7-bromo-3,4-dihydroisoquinolin-2(1H)-yl)(2,3,5,6-tetramethylphenyl)methanone (693.30 mg, 1.86 mmol, 78.91% yield). LCMS: RT=1.94 min, m/z=372.1.

### 3. Preparation of (7-(4,4,5,5-tetramethyl-1,3,2-dioxaborolan-2-yl)-3,4-dihydroisoquinolin-2(1H)-yl)(2,3,5,6-tetramethylphenyl)methanone

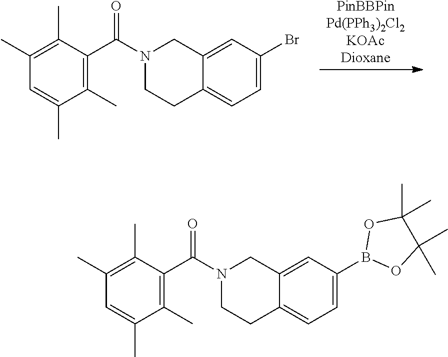

(7-Bromo-3,4-dihydroisoquinolin-2(1H)-yl)(2,3,5,6-tetramethylphenyl)methanone (693.30 mg, 1.86 mmol), 4,4,5,5-tetramethyl-2-(4,4,5,5-tetramethyl-1,3,2-dioxaborolan-2-yl)-1,3,2-dioxaborolane (708.49 mg, 2.79 mmol), dichloropalladium;triphenylphosphane (130.55 mg, 186.00 umol), potassium acetate (547.63 mg, 5.58 mmol) in dioxane (8.00 mL) was degassed and stirred at reflux for overnight. After dilution with EtOAc and filtration through celite, the concentrated crude was chromatographed on Si gel (HE/EA 0-100% Et/HE) to give (7-(4,4,5,5-tetramethyl-1,3,2-dioxaborolan-2-yl)-3,4-dihydroisoquinolin-2(1H)-yl)(2,3,5,6-tetramethylphenyl)methanone (770.00 mg, 1.74 mmol, 93.78% yield, 95% purity). LCMS: Rt=2.10 min, m/z=420.20.

### 4. Preparation of methyl 3-(6-methoxy-4-methyl-3-pyridyl)-3-[2-(2,3,5,6-tetramethylbenzoyl)-3,4-dihydro-1H-isoquinolin-7-yl]propanoate

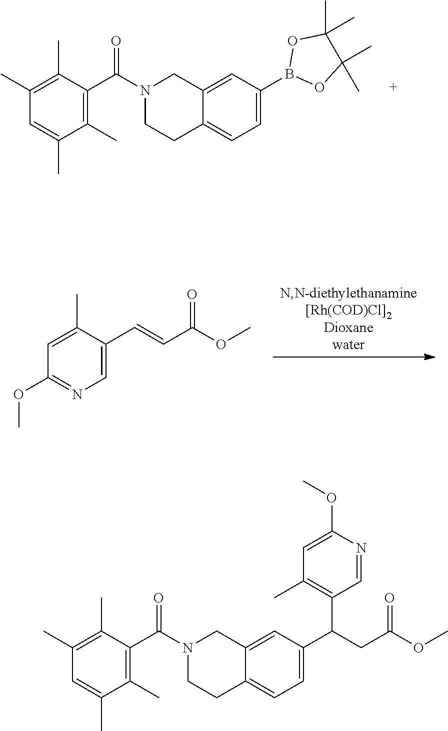

[7-(4,4,5,5-Tetramethyl-1,3,2-dioxaborolan-2-yl)-3,4-dihydro-1H-isoquinolin-2-yl]-(2,3,5,6-tetramethylphenyl)methanone (283.00 mg, 674.84 umol), methyl (E)-3-(6-methoxy-4-methyl-3-pyridyl)prop-2-enoate (93.23 mg, 449.89 umol), N,N-diethylethanamine (136.57 mg, 1.35 mmol, 187.09 uL) and [Rh(COD)Cl]₂ (12.92 mg, 44.99 umol) in Dioxane (1.50 mL) and water (500.00 uL) was microwaved at 120° C. for 50 min. After filtration through celite and washing with EtOAc, the concentrated organic was purified with prep HPLC to give methyl 3-(6-methoxy-4-methyl-3-pyridyl)-3-[2-(2,3,5,6-tetramethylbenzoyl)-3,4-dihydro-1H-isoquinolin-7-yl]propanoate (63.80 mg, 127.44 umol, 28.33% yield) LCMS: Rt=1.46, 1.54 min, m/z=501.3.

### 5. Preparation of 3-(6-methoxy-4-methyl-3-pyridyl)-3-[2-(2,3,5,6-tetramethylbenzoyl)-3,4-dihydro-1H-isoquinolin-7-yl]propanoic acid;

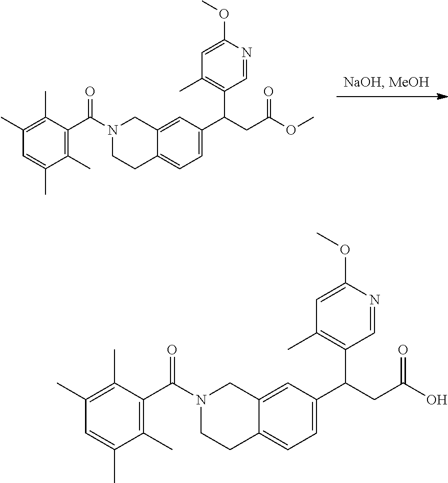

Methyl 3-(6-methoxy-4-methyl-3-pyridyl)-3-[2-(2,3,5,6-tetramethylbenzoyl)-3,4-dihydro-1H-isoquinolin-7-yl]propanoate (63.80 mg, 127.44 umol) in methanol (2.00 mL) was added sodium hydroxide (2 M, 191.16 uL) and microwaved at 100° C. for 30 min. After neutralized with 2M HCl, the crude was purified with prep HPLC to give 3-(6-methoxy-4-methyl-3-pyridyl)-3-[2-(2,3,5,6-tetramethylbenzoyl)-3,4-dihydro-1H-isoquinolin-7-yl]propanoic acid; (41.30 mg, 80.63 umol, 63.27% yield, 95% purity). LCMS: Rt=1.24, 1.36 min, m/z=487.3. ¹H NMR (400 MHz, CHLOROFORM-d) δ 8.21-8.47 (m, 1H), 6.50-7.19 (m, 5H), 3.92-5.10 (m, 9H), 2.56-3.67 (m, 4H), 2.40 (s, 3H), 1.91-2.27 (m, 12H).

### Example 57

### (3S)-3-(6-methoxy-4-methyl-3-pyridyl)-3-[2-(2,3,5,6-tetramethylbenzoyl)-3,4-dihydro-1H-isoquinolin-7-yl]propanoic acid; and (3R)-3-(6-methoxy-4-methyl-3-pyridyl)-3-[2-(2,3,5,6-tetramethylbenzoyl)-3,4-dihydro-1H-isoquinolin-7-yl]propanoic acid;

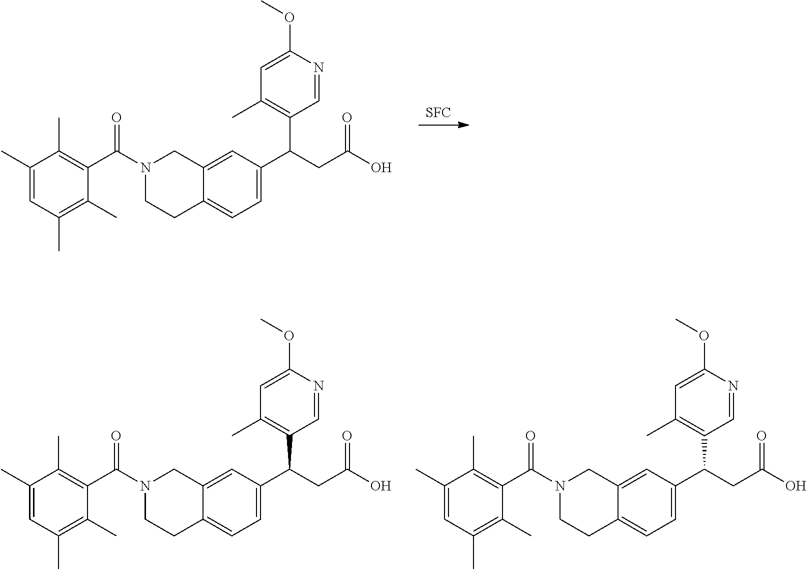

3-(6-Methoxy-4-methyl-3-pyridyl)-3-[2-(2,3,5,6-tetramethylbenzoyl)-3,4-dihydro-1H-isoquinolin-7-yl]propanoic acid; (37.00 mg, 76.04 umol) was separated under the following SFC condition: (Column: CHIRALPAK OX-H 30×250 mm, 5 um; Co-solvent: 40% Methanol in 0.1% DEA in CO2 (flow rate: 100 mL/min), ABPR 120 bar, MBPR 40 psi) to give the first eluate as Ent-1 (7.30 mg, 15.00 umol, 19.73% yield, 100% purity). LCMS: Rt=1.24, 1.36 min, m/z=487.3. ¹H NMR (400 MHz, CHLOROFORM-d) δ 7.42-8.32 (m, 1H), 6.27-7.20 (m, 5H), 2.50-5.26 (m, 15H), 1.88-2.36 (m, 12H); and the second eluate as Ent-2 (7.10 mg, 14.59 umol, 19.19% yield, 100% purity). LCMS: Rt=1.24, 1.36 min, m/z=487.3. ¹H NMR (400 MHz, CHLOROFORM-d) δ 7.42-8.32 (m, 1H), 6.27-7.20 (m, 5H), 2.50-5.26 (m, 15H), 1.88-2.36 (m, 12H).

### Example 58

### 3-(4-cyano-2-methyl-phenyl)-3-[2-(2,3,5,6-tetramethylbenzoyl)-3,4-dihydro-1H-isoquinolin-7-yl]propanoic acid; and 3-(4-carbamoyl-2-methyl-phenyl)-3-[2-(2,3,5,6-tetramethylbenzoyl)-3,4-dihydro-1H-isoquinolin-7-yl]propanoic acid;

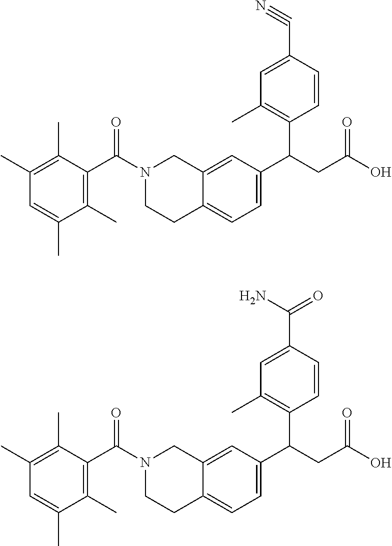 [SMILES: Cc1cc(C#N)ccc1C(CC(=O)O)c1ccc2c(c1)CN(C(=O)c1c(C)c(C)cc(C)c1C)CC2 ; Cc1cc(C(N)=O)ccc1C(CC(=O)O)c1ccc2c(c1)CN(C(=O)c1c(C)c(C)cc(C)c1C)CC2]

### 1. Preparation of methyl (E)-3-(4-cyano-2-methyl-phenyl)prop-2-enoate

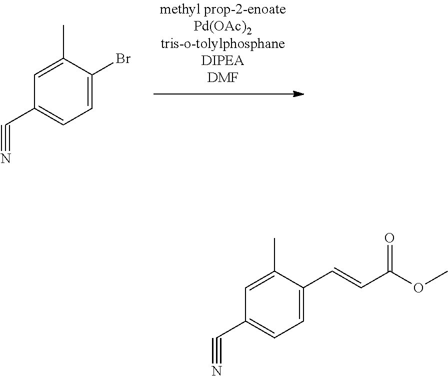

4-Bromo-3-methyl-benzonitrile (500.00 mg, 2.55 mmol), methyl prop-2-enoate (1.10 g, 12.75 mmol, 1.14 mL), Pd(OAc)₂ (57.25 mg, 255.00 umol), tris-o-tolylphosphane (155.23 mg, 510.00 umol) and DIPEA (988.69 mg, 7.65 mmol, 1.34 mL) in DMF (6.00 mL) was microwaved at 120° C. for 2 h. After dilution with EtOAc and washing with water, drying over Na₂SO₄, the crude was chromatographed on Si gel (HE/EA 0-100%) to give methyl (E)-3-(4-cyano-2-methyl-phenyl)prop-2-enoate (496.00 mg, 2.46 mmol, 96.67% yield). LCMS: Rt=1.42 min, m/z=202.1.

### 2. Preparation of methyl 3-(4-cyano-2-methyl-phenyl)-3-[2-(2,3,5,6-tetramethylbenzoyl)-3,4-dihydro-1H-isoquinolin-7-yl]propanoate

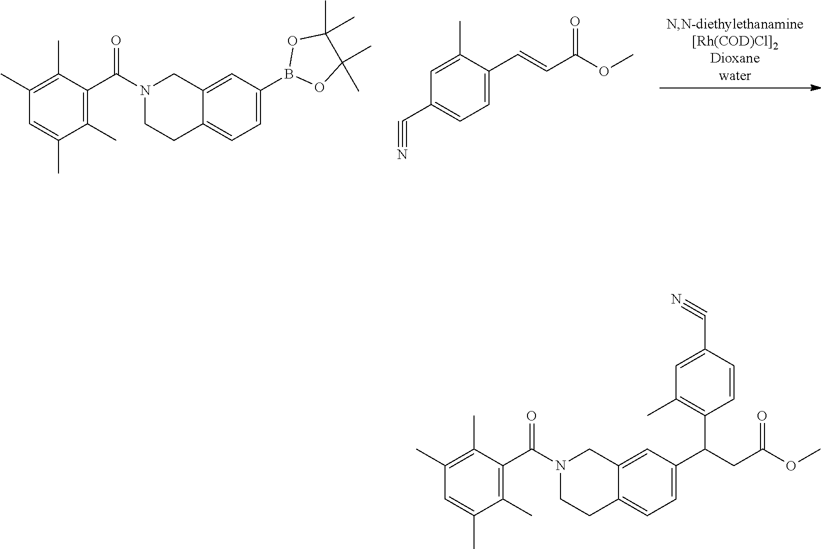

[7-(4,4,5,5-Tetramethyl-1,3,2-dioxaborolan-2-yl)-3,4-dihydro-1H-isoquinolin-2-yl]-(2,3,5,6-tetramethylphenyl)methanone (312.61 mg, 745.46 umol), methyl (E)-3-(4-cyano-2-methyl-phenyl)prop-2-enoate (100.00 mg, 496.97 umol), N,N-diethylethanamine (150.87 mg, 1.49 mmol, 206.67 uL) and [Rh(COD)Cl]₂ (14.28 mg, 49.70 umol) in Dioxane (1.50 mL) and water (500.00 uL) was degassed and microwaved at120° C. for 50 min. After dilution with EtOAc and filtration through Celite, the dried concentrated residue was chromatographed on Si gel (HE/EA 0-100%) to give methyl 3-(4-cyano-2-methyl-phenyl)-3-[2-(2,3,5,6-tetramethylbenzoyl)-3,4-dihydro-1H-isoquinolin-7-yl]propanoate (238.00 mg, 481.18 umol, 96.82% yield). LCMS: Rt=1.84, 1.96 min, m/z=495.3.

### 3. Preparation of 3-(4-carbamoyl-2-methyl-phenyl)-3-[2-(2,3,5,6-tetramethylbenzoyl)-3,4-dihydro-1H-isoquinolin-7-yl]propanoic acid; and 3-(4-cyano-2-methyl-phenyl)-3-[2-(2,3,5,6-tetramethylbenzoyl)-3,4-dihydro-1H-isoquinolin-7-yl]propanoic acid;

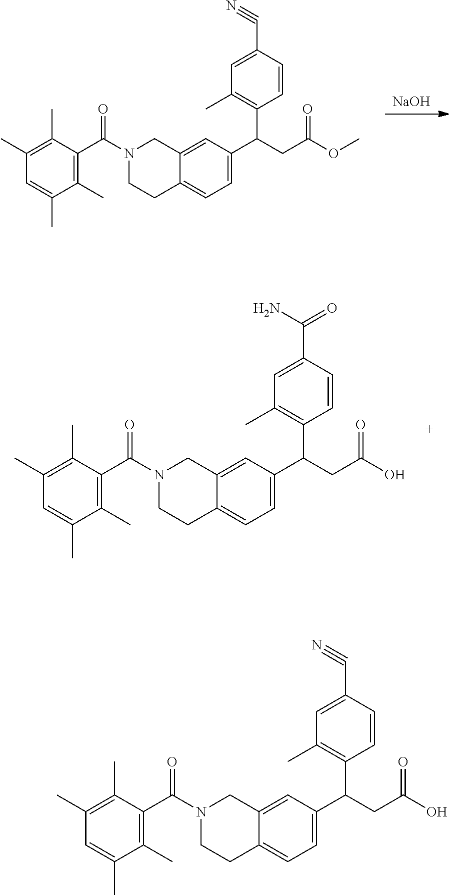

Methyl 3-(4-cyano-2-methyl-phenyl)-3-[2-(2,3,5,6-tetramethylbenzoyl)-3,4-dihydro-1H-isoquinolin-7-yl]propanoate (238.00 mg, 481.18 umol) in methanol (2.00 mL) was added sodium hydroxide (2 M, 240.59 uL) and microwaved at 100° C. for 30 min. After neutralized with 2M HCl, the crude was purified on prep HPLC to give compound 58-1, 3-(4-cyano-2-methyl-phenyl)-3-[2-(2,3,5,6-tetramethylbenzoyl)-3,4-dihydro-1H-isoquinolin-7-yl]propanoic acid; as separable atropisomers (18.80 mg, 7% yield, 95% purity). LCMS: Rt=1.77, 1.60 min, m/z=481.2. Peak3: ¹H NMR (400 MHz, CHLOROFORM-d) δ 7.35-7.64 (m, 2H), 7.13 (d, J=8.03 Hz, 1H), 6.92-7.08 (m, 3H), 6.64 (s, 1H), 4.66 (t, J=7.78 Hz, 1H), 4.23 (s, 2H), 3.93-4.16 (m, 2H), 2.86-3.06 (m, 4H), 2.30 (s, 3H), 2.21 (s, 6H), 1.99 (s, 6H). Peak 4: ¹H NMR (400 MHz, CHLOROFORM-d) δ 7.52-7.67 (m, 1H), 7.47 (s, 1H), 7.38 (d, J=8.03 Hz, 1H), 6.91-7.12 (m, 4H), 4.94 (s, 2H), 4.75 (t, J=7.78 Hz, 1H), 3.42 (t, J=5.90 Hz, 2H), 2.97-3.13 (m, 2H), 2.72 (t, J=5.65 Hz, 2H), 2.37 (s, 3H), 2.22 (s, 6H), 2.08 (s, 6H). And side product 58-2, 3-(4-carbamoyl-2-methyl-phenyl)-3-[2-(2,3,5,6-tetramethylbenzoyl)-3,4-dihydro-1H-isoquinolin-7-yl]propanoic acid; (16.60 mg, 7% yield, 95% purity) as separable atropisomers. LCMS: Rt=1.27, 1.45 min, m/z=499.2. Peak 1: ¹H NMR (400 MHz, CHLOROFORM-d) δ 7.51-7.76 (m, 2H), 7.30 (d, J=7.53 Hz, 1H), 7.08-7.17 (m, 1H), 6.90-7.06 (m, 2H), 6.65 (s, 1H), 4.67 (t, J=7.91 Hz, 1H), 4.22 (s, 2H), 3.90-4.15 (m, 2H), 2.76-3.14 (m, 4H), 2.31 (s, 3H), 2.20 (s, 6H), 1.98 (br. s., 6H). Peak2: 1H NMR (400 MHz, CHLOROFORM-d) δ 7.54-7.76 (m, 2H), 7.39 (d, J=8.03 Hz, 1H), 6.87-7.12 (m, 4H), 4.93 (s, 2H), 4.77 (t, J=7.91 Hz, 1H), 3.41 (t, J=5.77 Hz, 2H), 2.96-3.24 (m, 2H), 2.73 (br. s., 2H), 2.38 (s, 3H), 2.22 (s, 6H), 2.08 (d, J=2.01 Hz, 6H).

### Example 59

### 3-(2-benzoyl-3,4-dihydro-1H-isoquinolin-7-yl)-3-(4-cyano-2-methyl-phenyl)propanoic acid;

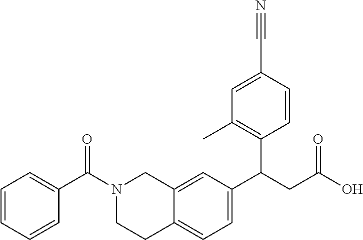 [SMILES: Cc1cc(C#N)ccc1C(CC(=O)O)c1ccc2c(c1)CN(C(=O)c1ccccc1)CC2]

### 1. Preparation of methyl 3-(2-benzoyl-3,4-dihydro-1H-isoquinolin-7-yl)-3-(4-cyano-2-methyl-phenyl)propanoate

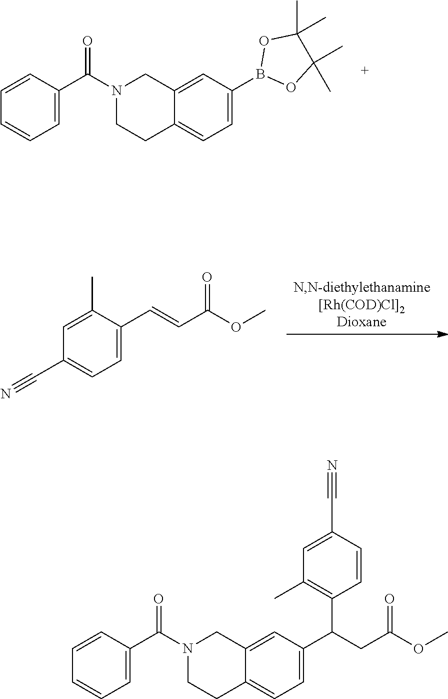

Phenyl-[7-(4,4,5,5-tetramethyl-1,3,2-dioxaborolan-2-yl)-3,4-dihydro-1H-isoquinolin-2-yl]methanone (128.00 mg, 352.36 umol), methyl (E)-3-(4-cyano-2-methyl-phenyl)prop-2-enoate (47.27 mg, 234.91 umol), N,N-diethylethanamine (71.31 mg, 704.72 umol, 97.68 uL) and [Rh(COD)Cl]₂ (6.75 mg, 23.49 umol) in Dioxane (1 mL) and water (300 uL) was microwaved at 120° C. for 50 min. After filtration through celite, the crude was purified through prep HPLC to give methyl 3-(2-benzoyl-3,4-dihydro-1H-isoquinolin-7-yl)-3-(4-cyano-2-methyl-phenyl)propanoate (53.50 mg, 122.00 umol, 51.94% yield). LCMS: Rt=1.67 min, m/z=439.1.

### 2. Preparation of 3-(2-benzoyl-3,4-dihydro-1H-isoquinolin-7-yl)-3-(4-cyano-2-methyl-phenyl)propanoic acid;

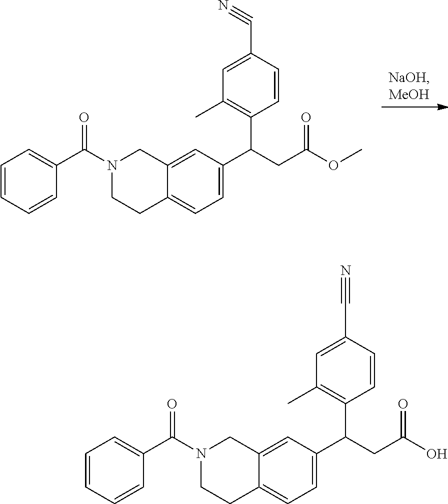

Methyl 3-(2-benzoyl-3,4-dihydro-1H-isoquinolin-7-yl)-3-(4-cyano-2-methyl-phenyl)propanoate (53.50 mg, 118.22 umol) in methanol (2.00 mL) was added sodium hydroxide (2 M, 118.22 uL) and microwaved at 100° C. for 30 min. After neutralized with 2M HCl, the crude was purified with prep HPLC to give 3-(2-benzoyl-3,4-dihydro-1H-isoquinolin-7-yl)-3-(4-cyano-2-methyl-phenyl)propanoic acid; (35.70 mg, 84.10 umol, 71.14% yield). LCMS: Rt=1.25 min, m/z=425.1. ¹H NMR (400 MHz, CHLOROFORM-d) δ 7.31-7.74 (m, 8H), 6.58-7.21 (m, 3H), 2.58-6.03 (m, 9H), 2.36 (br. s., 3H).

### Example 60

### 3-[(1R)-2-benzoyl-1-methyl-3,4-dihydro-1H-isoquinolin-7-yl]-3-(1-ethyl-4-methyl-benzotriazol-5-yl)propanoic acid;

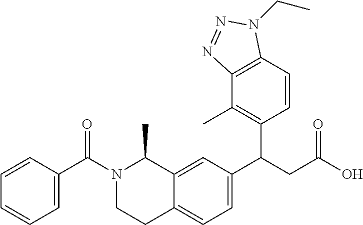 [SMILES: CCn1nnc2c(C)c(C(CC(=O)O)c3ccc4c(c3)[C@H](C)N(C(=O)c3ccccc3)CC4)ccc21]

### 1. Preparation of [(1S)-7-bromo-1-methyl-3,4-dihydro-1H-isoquinolin-2-yl]-phenyl-methanon

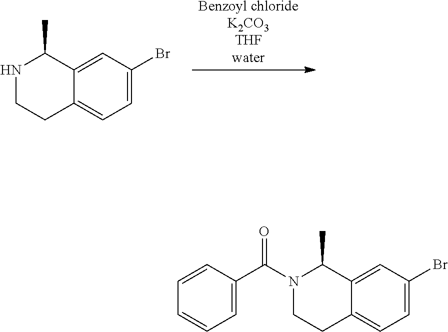 [SMILES: C[C@@H]1NCCc2ccc(Br)cc21 ; "Benzoyl chlorideK2CO3THFwater" ; C[C@H]1c2cc(Br)ccc2CCN1C(=O)c1ccccc1]

(1S)-7-bromo-1-methyl-1,2,3,4-tetrahydroisoquinoline (500.00 mg, 1.90 mmol, Hydrochloride), benzoyl chloride (267.68 mg, 1.90 mmol, 221.22 uL), K₂CO₃ (789.56 mg, 5.71 mmol) in THF (5.00 mL) and water (499.95 uL) was stirred at rt for overnight. After dilution with EtOAc and washing with water, the dried crude was purified on Si gel (HE/EA 0-100%) to give [(1S)-7-bromo-1-methyl-3,4-dihydro-1H-isoquinolin-2-yl]-phenyl-methanone (628.00 mg, 1.90 mmol, 100.09% yield). LCMS: Rt=1.68 min, m/z=330.0.

### 2. Preparation of (S)-(1-methyl-7-(4,4,5,5-tetramethyl-1,3,2-dioxaborolan-2-yl)-3,4-dihydroisoquinolin-2(1H)-yl)(phenyl)methanone

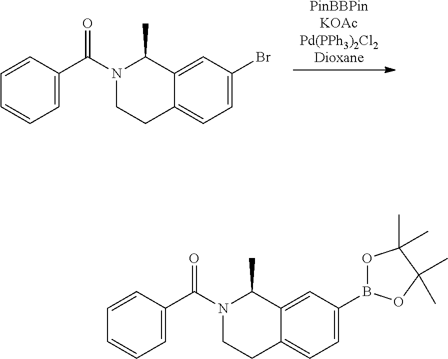 [SMILES: C[C@H]1c2cc(Br)ccc2CCN1C(=O)c1ccccc1 ; "PinBBPinKOAcPd(PPh3)2Cl2Dioxane" ; C[C@H]1c2cc(B3OC(C)(C)C(C)(C)O3)ccc2CCN1C(=O)c1ccccc1]

[(1S)-7-bromo-1-methyl-3,4-dihydro-1H-isoquinolin-2-yl]-phenyl-methanone (729.00 mg, 2.21 mmol), 4,4,5,5-tetramethyl-2-(4,4,5,5-tetramethyl-1,3,2-dioxaborolan-2-yl)-1,3,2-dioxaborolane (560.60 mg, 2.21 mmol), potassium acetate (649.97 mg, 6.62 mmol) and dichloropalladium;triphenylphosphane (154.95 mg, 220.76 umol) in Dioxane (4.00 mL) was refluxed for overnight. After dilution with EtOAc and filtration through celite, the crude was chromatographed on Si gel (HE/EA 0-100%) to give (S)-(1-methyl-7-(4,4,5,5-tetramethyl-1,3,2-dioxaborolan-2-yl)-3,4-dihydroisoquinolin-2(1H)-yl)(phenyl)methanone (891.00 mg, 2.36 mmol, 106.86% yield). (LCMS: Rt=1.85 min, m/z=378.20.

### 3. Preparation of methyl 3-[(1S)-2-benzoyl-1-methyl-3,4-dihydro-1H-isoquinolin-7-yl]-3-(1-ethyl-4-methyl-benzotriazol-5-yl)propanoate

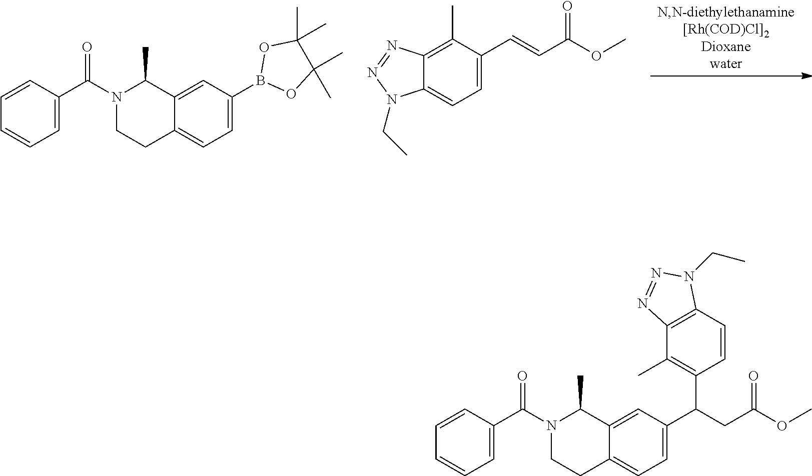

[(1S)-1-methyl-7-(4,4,5,5-tetramethyl-1,3,2-dioxaborolan-2-yl)-3,4-dihydro-1H-isoquinolin-2-yl]-phenyl-methanone (281.48 mg, 746.09 umol), methyl (E)-3-(1-ethyl-4-methyl-benzotriazol-5-yl)prop-2-enoate (122.00 mg, 497.39 umol), N,N-diethylethanamine (150.99 mg, 1.49 mmol, 206.84 uL), [Rh(COD)Cl]₂ (14.29 mg, 49.74 umol) in Dioxane (1.50 mL) and water (500.00 uL) was microwaved at 150° C. for 50 min. After dilution with EtOAc and filtration through celite, the dried concentrated crude was purified with prep HPLC to give methyl 3-[(1S)-2-benzoyl-1-methyl-3,4-dihydro-1H-isoquinolin-7-yl]-3-(1-ethyl-4-methyl-benzotriazol-5-yl)propanoate (58.00 mg, 110.95 umol, 22.31% yield, 95% purity). LCMS: Rt=1.61 min, m/z=497.2.

### 4. Preparation of 3-[(1R)-2-benzoyl-1-methyl-3,4-dihydro-1H-isoquinolin-7-yl]-3-(1-ethyl-4-methyl-benzotriazol-5-yl)propanoic acid;

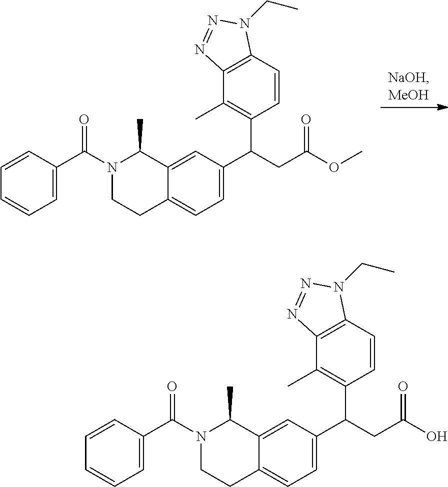

Methyl 3-[(1R)-2-benzoyl-1-methyl-3,4-dihydro-1H-isoquinolin-7-yl]-3-(1-ethyl-4-methyl-benzotriazol-5-yl)propanoate (58.00 mg, 116.79 umol) in methanol (2.00 mL) was added sodium hydroxide (2 M, 116.79 uL) and microwaved at 100° C. for 30 min. After neutralization with 2M HCl, the crude was purified with prep HPLC to give 3-[(1R)-2-benzoyl-1-methyl-3,4-dihydro-1H-isoquinolin-7-yl]-3-(1-ethyl-4-methyl-benzotriazol-5-yl)propanoic acid; (47.00 mg, 92.53 umol, 79.22% yield, 95% purity). LCMS: Rt=1.41 min, m/z=483.2. ¹H NMR (400 MHz, CHLOROFORM-d) δ 7.30-7.62 (m, 7H), 6.57-7.16 (m, 3H), 4.45-5.90 (m, 4H), 2.53-4.04 (m, 9H), 1.21-1.82 (m, 6H).

### Example 61

### 3-(2-benzoyl-1,3,4,5-tetrahydro-2-benzazepin-8-yl)-3-(1-ethyl-4-methyl-benzotriazol-5-yl)propanoic acid;

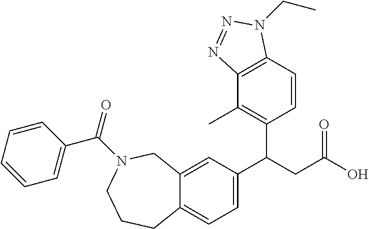 [SMILES: CCn1nnc2c(C)c(C(CC(=O)O)c3ccc4c(c3)CN(C(=O)c3ccccc3)CCC4)ccc21]

### 1. Preparation of (8-bromo-1,3,4,5-tetrahydro-2-benzazepin-2-yl)-phenyl-methanone

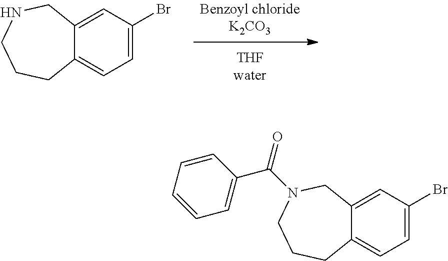 [SMILES: Brc1ccc2c(c1)CNCCC2 ; O=C(c1ccccc1)N1CCCc2ccc(Br)cc2C1]

8-Bromo-2,3,4,5-tetrahydro-1H-2-benzazepine (500.00 mg, 2.21 mmol), K₂CO₃ (916.33 mg, 6.63 mmol) in THF (5.00 mL) and water (500 uL) was added Benzoyl chloride (341.85 mg, 2.43 mmol, 282.52 uL) and stirred at rt for overnight. After dilution with EtOAc and washing with water, brine, the concentrated residue was chromatographed on Si gel (HE/EA 0-100%) to give (8-bromo-1,3,4,5-tetrahydro-2-benzazepin-2-yl)-phenyl-methanone (717.00 mg, 2.17 mmol, 98.25% yield).

### 2. Preparation of phenyl(8-(4,4,5,5-tetramethyl-1,3,2-dioxaborolan-2-yl)-1,3,4,5-tetrahydro-2H-benzo[c]azepin-2-yl)methanone

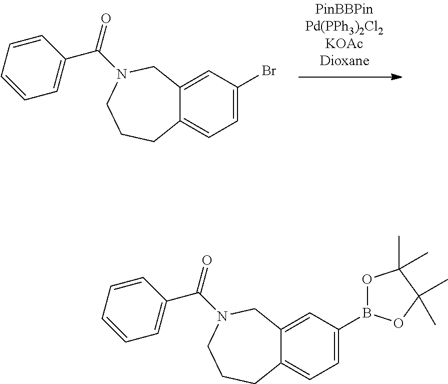 [SMILES: O=C(c1ccccc1)N1CCCc2ccc(Br)cc2C1 ; CC1(C)OB(c2ccc3c(c2)CN(C(=O)c2ccccc2)CCC3)OC1(C)C]

(8-Bromo-1,3,4,5-tetrahydro-2H-benzo[c]azepin-2-yl)(phenyl)methanone (717.00 mg, 2.17 mmol), 4,4,5,5-tetramethyl-2-(4,4,5,5-tetramethyl-1,3,2-dioxaborolan-2-yl)-1,3,2-dioxaborolane (826.57 mg, 3.26 mmol), dichloropalladium;triphenylphosphane (152.31 mg, 217.00 umol), Potassium acetate (638.91 mg, 6.51 mmol) in Dioxane (8.00 mL) was heated at 100° C. for overnight. After dilution with EtOAc and filtration through celite, the concentrated residue was purified through chromatograph on Si gel (HE/EA 0-100%) to give phenyl(8-(4,4,5,5-tetramethyl-1,3,2-dioxaborolan-2-yl)-1,3,4,5-tetrahydro-2H-benzo[c]azepin-2-yl)methanone (800.00 mg, 2.12 mmol, 97.72% yield). LCMS: rt=1.82 min, m/z=378.20.

### 3. Preparation of methyl 3-(2-benzoyl-1,3,4,5-tetrahydro-2-benzazepin-8-yl)-3-(1-ethyl-4-methyl-benzotriazol-5-yl)propanoate

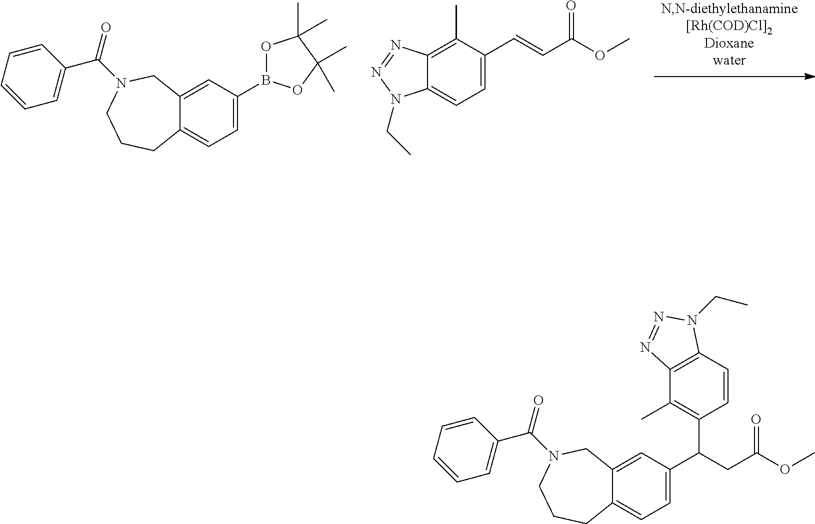

Phenyl-[8-(4,4,5,5-tetramethyl-1,3,2-dioxaborolan-2-yl)-1,3,4,5-tetrahydro-2-benzazepin-2-yl]methanone (294.00 mg, 779.26 umol), methyl (E)-3-(1-ethyl-4-methyl-benzotriazol-5-yl)prop-2-enoate (127.42 mg, 519.51 umol), N,N-diethylethanamine (157.71 mg, 1.56 mmol, 216.04 uL) and [Rh(COD)Cl]₂ (25.62 mg, 51.95 umol) in Dioxane (3.00 mL) and water (1.00 mL) was microwaved at 150° C. for 50 min. After dilution with EtOAc and filtration through celite, the brine washed dried residue was purified with prep HPLC to give methyl 3-(2-benzoyl-1,3,4,5-tetrahydro-2-benzazepin-8-yl)-3-(1-ethyl-4-methyl-benzotriazol-5-yl)propanoate (30.70 mg, 61.82 umol, 11.90% yield). LCMS: rt=1.56 min, m/z=497.2.

### 4. Preparation of 3-(2-benzoyl-1,3,4,5-tetrahydro-2-benzazepin-8-yl)-3-(1-ethyl-4-methyl-benzotriazol-5-yl)propanoic acid;

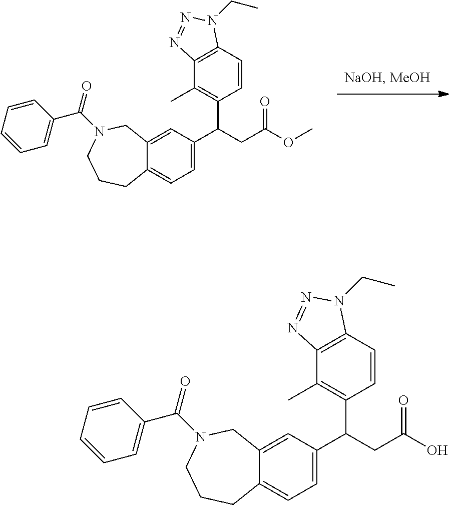 [SMILES: CCn1nnc2c(C)c(C(CC(=O)OC)c3ccc4c(c3)CN(C(=O)c3ccccc3)CCC4)ccc21 ; CCn1nnc2c(C)c(C(CC(=O)O)c3ccc4c(c3)CN(C(=O)c3ccccc3)CCC4)ccc21]

Methyl 3-(2-benzoyl-1,3,4,5-tetrahydro-2-benzazepin-8-yl)-3-(1-ethyl-4-methyl-benzotriazol-5-yl)propanoate (30.70 mg, 61.82 umol) in methanol (2.00 mL) was added sodium hydroxide (2 M, 61.82 uL) and was microwaved at 100° C. for 30 min. After neutralization with 2M HCl, the crude was purified with prep HPLC to give 3-(2-benzoyl-1,3,4,5-tetrahydro-2-benzazepin-8-yl)-3-(1-ethyl-4-methyl-benzotriazol-5-yl)propanoic acid; (25.50 mg, 50.20 umol, 81.20% yield, 95% purity). LCMS: Rt=1.36 min, m/z=483.2. ¹H NMR (400 MHz, CHLOROFORM-d) δ 7.29-7.60 (m, 6H), 6.72-7.25 (m, 4H), 2.90-5.38 (m, 11H), 2.64-2.88 (m, 3H), 1.70-2.35 (m, 2H), 1.48-1.68 (m, 3H).

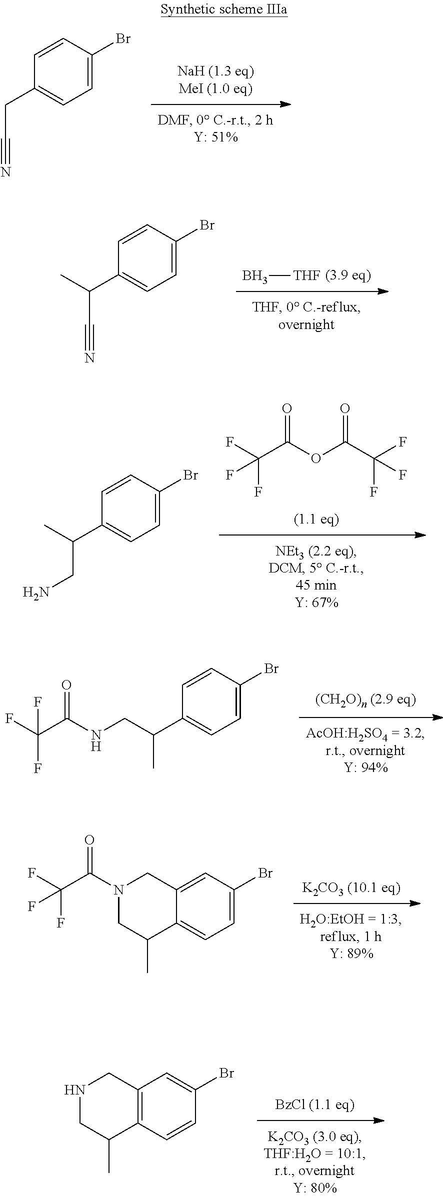

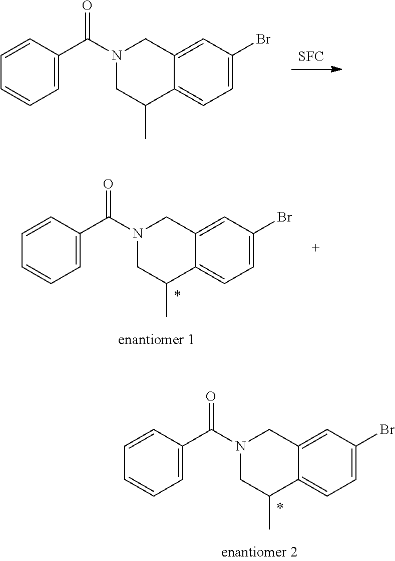

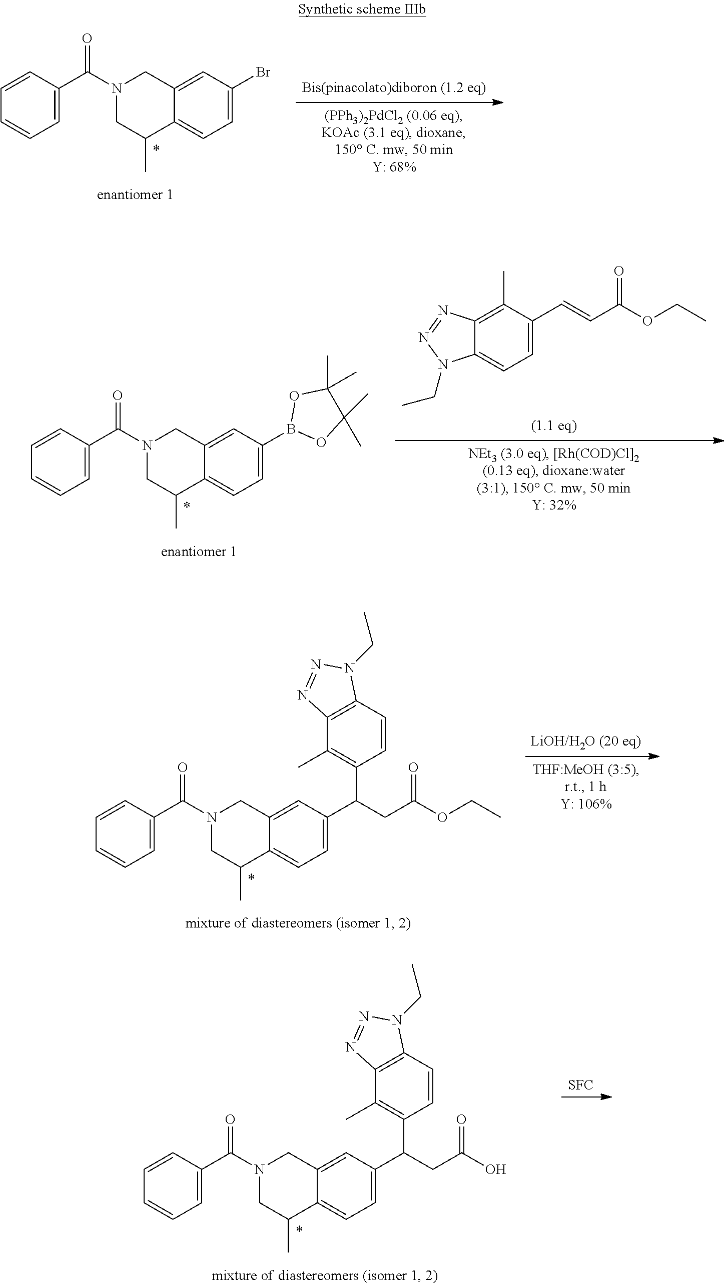

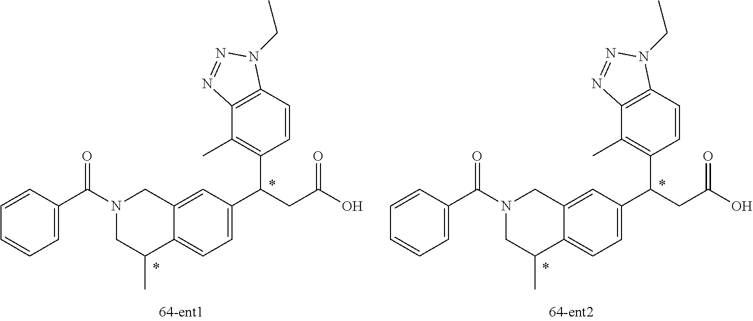

### Example 64

### 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 1 [64-ent1] mono DEA salt and 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 2 [64-ent2] mono DEA salt

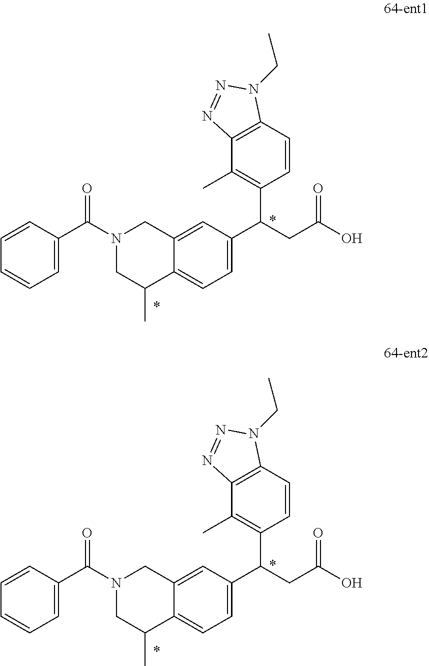 [SMILES: CCn1nnc2c(C)c(C(CC(=O)O)c3ccc4c(c3)CN(C(=O)c3ccccc3)CC4C)ccc21 ; CCn1nnc2c(C)c(C(CC(=O)O)c3ccc4c(c3)CN(C(=O)c3ccccc3)CC4C)ccc21]

### 1. The Preparation of 2-(4-bromophenyl)propanenitrile

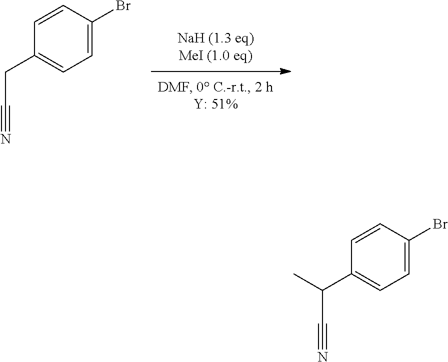

In a round bottom flask, 2-(4-bromophenyl)acetonitrile (1.01 g, 5.15 mmol) was dissolved in DMF (5.15 mL). At 0° C. was added 60% sodium hydride (261 mg, 6.53 mmol, 1.27 eq) and methyl iodide (321 uL, 5.15 mmol, 1.0 eq) was added over 30 min. After 2 h stirring at RT, reaction was quenched by addition to cold water. Mixture was extracted with ethyl acetate, washed with saturated sodium chloride, dried over magnesium sulfate, filtered, evaporated. Purification by silica gel chromatography (0-50% ethyl acetate in heptanes as eluent) yielded 2-(4-bromophenyl)propanenitrile (553.5 mg, 51.2% yield). ¹H NMR (400 MHz, CHLOROFORM-d) δ 7.53 (d, J=8.53 Hz, 2H), 7.25 (d, J=8.28 Hz, 2H), 3.88 (q, J=7.28 Hz, 1H), 1.64 (d, J=7.28 Hz, 3H).

### 2. The Preparation of 2-(4-bromophenyl)propan-1-amine

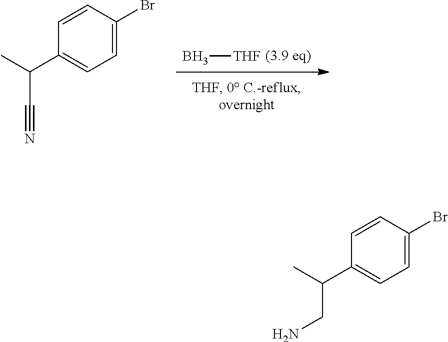

To 2-(4-bromophenyl)propanenitrile (1.39 g, 6.62 mmol) in THF (17 mL) was added borane-THF (1 M in THF, 26.00 mL, 26.0 mmol, 3.9 eq) at 0° C. The reaction was heated to reflux overnight. After cooling, 4M HCl in dioxane was added to pH 2. Evaporation was followed by dissolution in methanol and evaporation (twice). The crude 2-(4-bromophenyl)propan-1-amine was carried on to the next step without further treatment. ESI-MS (M+H)⁺: 214.0/216.0. ¹H NMR (400 MHz, METHANOL-d4) δ 7.53 (d, J=8.28 Hz, 2H), 7.24 (d, J=8.28 Hz, 2H), 3.10-3.15 (m, 2H), 2.99-3.09 (m, 1H), 1.33 (d, J=6.78 Hz, 3H).

### 3. The Preparation of N-[2-(4-bromophenyl)propyl]-2,2,2-trifluoro-acetamide

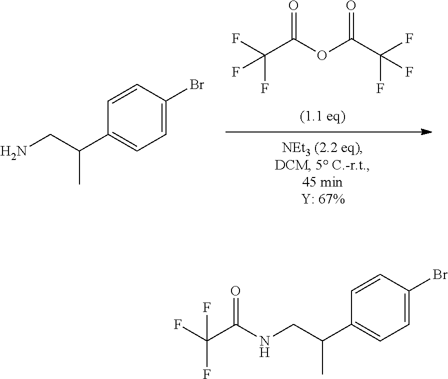

To a solution of 2-(4-bromophenyl)propan-1-amine (6.62 mmol) and triethylamine (2.01 mL, 14.50 mmol, 2.2 eq) in DCM (25 mL) was added trifluoroacetic acid; anhydride (1.00 mL, 7.22 mmol, 1.1 eq) at 5° C. dropwise and with vigorous stirring. After 45 min at RT, the reaction was poured into ice-water and extracted with DCM. The organic layer was washed with water, dried over anhydrous magnesium sulfate, filtered and concentrated in vacuo. Purification by preparative HPLC gave N-[2-(4-bromophenyl)propyl]-2,2,2-trifluoro-acetamide (1.37 g, 66.73% yield). ESI-MS (M+H)⁺: 310.0. ¹H NMR (400 MHz, CHLOROFORM-d) δ 7.49 (d, J=8.28 Hz, 2H), 7.09 (d, J=8.53 Hz, 2H), 6.09 (br. s., 1H), 3.67 (td, J=6.59, 13.43 Hz, 1H), 3.35 (ddd, J=5.27, 8.47, 13.62 Hz, 1H), 2.95-3.06 (m, 1H), 1.31 (d, J=7.03 Hz, 3H).

### 4. The Preparation of 1-(7-bromo-4-methyl-3,4-dihydro-1H-isoquinolin-2-yl)-2,2,2-trifluoro-ethanone

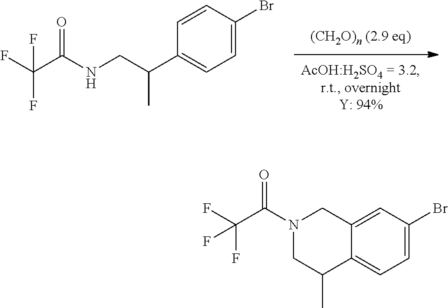 [SMILES: CC(CNC(=O)C(F)(F)F)c1ccc(Br)cc1 ; CC1CN(C(=O)C(F)(F)F)Cc2cc(Br)ccc21]

N-[2-(4-bromophenyl)propyl]-2,2,2-trifluoro-acetamide (677.2 mg, 2.18 mmol) and paraformaldehyde (191.0 mg, 6.36 mmol, 2.9 eq) were dissolved in premixed solution of acetic acid; (3.6 mL) and sulfuric acid; (2.4 mL). The reaction was stirred overnight, then poured into cold water, which was then extracted with ethyl acetate. The organic layer was washed with saturated sodium bicarbonate, then with water, then with saturated sodium chloride. The solution was dried over magnesium sulfate, filtered and evaporated to yield 1-(7-bromo-4-methyl-3,4-dihydro-1H-isoquinolin-2-yl)-2,2,2-trifluoro-ethanone (658.4 mg, 93.8% yield). ESI-MS(M+H)⁺: 322.0. ¹H NMR (400 MHz, CHLOROFORM-d) δ 7.36-7.42 (m, 1H), 7.28-7.33 (m, 1H), 7.12 (t, J=7.80 Hz, 1H), 4.77-4.90 (m, 1H), 4.62-4.72 (m, 1H), 3.70-4.13 (m, 2H), 3.37-3.67 (m, 1H), 1.30 (d, J=7.03 Hz, 3H).

### 5. The Preparation of 7-bromo-4-methyl-1,2,3,4-tetrahydroisoquinoline

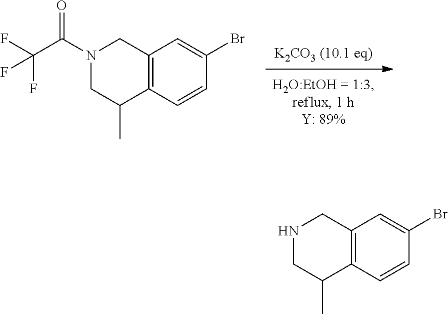 [SMILES: CC1CN(C(=O)C(F)(F)F)Cc2cc(Br)ccc21 ; CC1CNCc2cc(Br)ccc21]

1-(7-Bromo-4-methyl-3,4-dihydro-1H-isoquinolin-2-yl)-2,2,2-trifluoro-ethanone (658.4 mg, 2.04 mmol) was dissolved in ethyl alcohol (21.3 mL) and a solution of potassium carbonate (2.84 g, 20.54 mmol, 10.1 eq) in water (7.1 mL) was added. The mixture was heated to reflux for 1 hour then cooled and evaporated in vacuo. Water was added to the residue and extracted three times with DCM. The combined DCM extracts were washed with water, dried over MgSO4, filtered and concentrated in vacuo to give the crude product 7-bromo-4-methyl-1,2,3,4-tetrahydroisoquinoline (410.0 mg, 88.9% yield). ESI-MS(M+H)⁺: 226.0/228.0. Sample used as-is in subsequent reaction.

### 6. The Preparation of (7-bromo-4-methyl-3,4-dihydro-1H-isoquinolin-2-yl)-phenyl-methanone

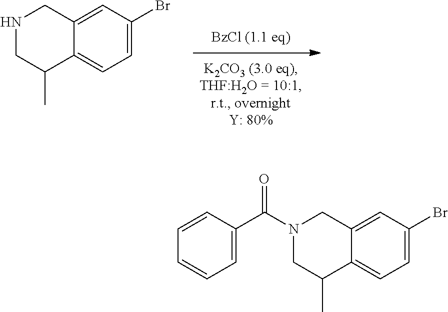 [SMILES: CC1CNCc2cc(Br)ccc21 ; CC1CN(C(=O)c2ccccc2)Cc2cc(Br)ccc21]

7-Bromo-4-methyl-1,2,3,4-tetrahydroisoquinoline (410.0 mg, 1.81 mmol) and potassium carbonate (751.8 mg, 5.44 mmol, 3.0 eq) were slurried in THF (5.01 mL) and water (501 uL) and to this was added benzoyl chloride (232 uL, 1.99 mmol, 1.1 eq) and the reaction was stirred at RT overnight. After dilution with ethyl acetate, the reaction was washed with water, then with brine, then dried with MgSO4, filtered and evaporated. The residue was purified with silica gel chromatography (using 0-50% ethyl acetate in heptanes as eluent) to give (7-bromo-4-methyl-3,4-dihydro-1H-isoquinolin-2-yl)-phenyl-methanone (479.6 mg, 80.2% yield). ESI-MS(M+H)⁺: 330.0.

### 7. The Preparation of (7-bromo-4-methyl-3,4-dihydro-1H-isoquinolin-2-yl)-phenyl-methanone enantiomer 1 and (7-bromo-4-methyl-3,4-dihydro-1H-isoquinolin-2-yl)-phenyl-methanone enantiomer 2

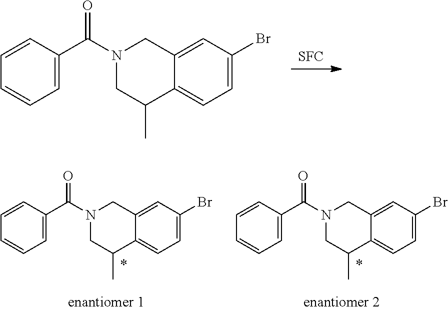

(7-Bromo-4-methyl-3,4-dihydro-1H-isoquinolin-2-yl)-phenyl-methanone (1.48 g, 4.48 mmol) was separated by preparative SFC chromatography (Column: CHIRALPAK AD-H 30×250 mm, 5 um; Co-solvent: 45% Methanol w/0.1% DEA in CO2 (flow rate: 100 mL/min), ABPR 120 bar, MBPR 40 psi) to yield: PEAK 1: (7-bromo-4-methyl-3,4-dihydro-1H-isoquinolin-2-yl)-phenyl-methanone enantiomer 1 (479.0 mg, 1.45 mmol, 32.4% yield, 100% ee purity), ESI-MS(M+H)⁺: 330.1). PEAK 2: (7-bromo-4-methyl-3,4-dihydro-1H-isoquinolin-2-yl)-phenyl-methanone enantiomer 2 (514.3 mg, 1.56 mmol, 34.8% yield, 98.96% ee purity), ESI-MS(M+H)⁺: 330.1.

### 8. The Preparation of (4-methyl-7-(4,4,5,5-tetramethyl-1,3,2-dioxaborolan-2-yl)-3,4-dihydroisoquinolin-2(1H)-yl)(phenyl)methanone enantiomer 1

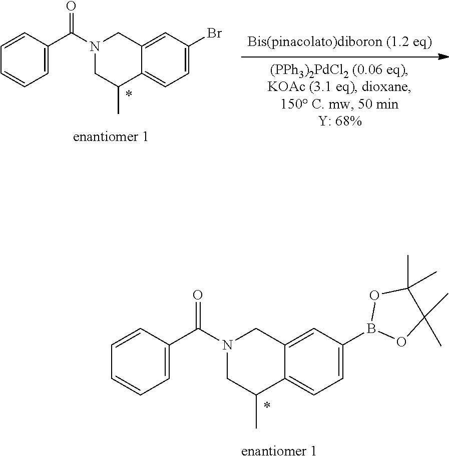 [SMILES: CC1CN(C(=O)c2ccccc2)Cc2cc(Br)ccc21 ; "(PPh3)2PdCl2 (0.06 eq),KOAc (3.1 eq), dioxane,150° C. mw, 50 minY: 68%" ; "Bis(pinacolato)diboron (1.2 eq)" ; CC1CN(C(=O)c2ccccc2)Cc2cc(B3OC(C)(C)C(C)(C)O3)ccc21]

(7-Bromo-4-methyl-3,4-dihydro-1H-isoquinolin-2-yl)-phenyl-methanone enantiomer 1 (291.9 mg, 884.0 umol) and bis(pinacolato)diboron (276.8 mg, 1.09 mmol, 1.2 eq) and bis(triphenylphosphine)palladium(II) dichloride (38.3 mg, 54.5 umol, 0.06 eq) and potassium acetate (267.5 mg, 2.73 mmol, 3.1 eq) were dissolved in dioxane (3.17 mL). After degassing, the reaction was sealed and microwaved at 150° C. for 50 min. The reaction was diluted with ethyl acetate, washed with water, washed with saturated sodium chloride, dried over magnesium sulfate, filtered. After evaporation, the residue was chromatographed on silica gel (0-100% ethyl acetate in heptanes as eluent) to give (4-methyl-7-(4,4,5,5-tetramethyl-1,3,2-dioxaborolan-2-yl)-3,4-dihydroisoquinolin-2(1H)-yl)(phenyl)methanone enantiomer 1 (228.1 mg, 68.4% yield). ESI-MS(M+H)⁺: 378.2.

### 9. The Preparation of ethyl (E)-3-(1-ethyl-4-methyl-benzotriazol-5-yl)prop-2-enoate

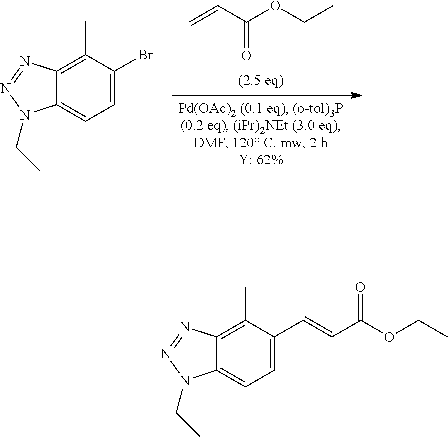

5-Bromo-1-ethyl-4-methyl-benzotriazole (5.00 g, 20.82 mmol) and palladium(II) acetate (467.5 mg, 2.08 mmol, 0.1 eq) and tri(o-tolyl)phosphine (1.27 g, 4.16 mmol, 0.2 eq) and diisopropylethylamine (10.91 mL, 62.46 mmol, 3.0 eq) and ethyl acrylate (5.66 mL, 52.05 mmol, 2.5 eq) were dissolved in DMF (4 vessels, 50 mL total) which was then degassed and microwaved at 120° C. for 2 h. Reactions were combined, diluted with ethyl acetate, washed with water, washed with saturated sodium chloride, dried over magnesium sulfate, filtered and concentrated to give a residue which was chromatographed (0-50% ethyl acetate in heptanes as eluent) to give ethyl (E)-3-(1-ethyl-4-methyl-benzotriazol-5-yl)prop-2-enoate (3.34 g, 61.9% yield). ESI-MS(M+H)⁺: 260.1. ¹H NMR (400 MHz, CHLOROFORM-d) δ 8.15 (d, J=16.06 Hz, 1H), 7.71 (d, J=8.78 Hz, 1H), 7.36 (d, J=8.53 Hz, 1H), 6.42 (d, J=16.06 Hz, 1H), 4.68 (q, J=7.28 Hz, 2H), 4.30 (q, J=7.11 Hz, 2H), 2.93 (s, 3H), 1.64 (t, J=7.28 Hz, 3H), 1.37 (t, J=7.15 Hz, 3H).

### 10. The Preparation of ethyl 3-(2-benzoyl-4-methyl-1,2,3,4-tetrahydroisoquinolin-7-yl)-3-(1-ethyl-4-methyl-1H-benzo[d][1,2,3]triazol-5-yl)propanoate mixture of diastereomers (isomer 1, 2)

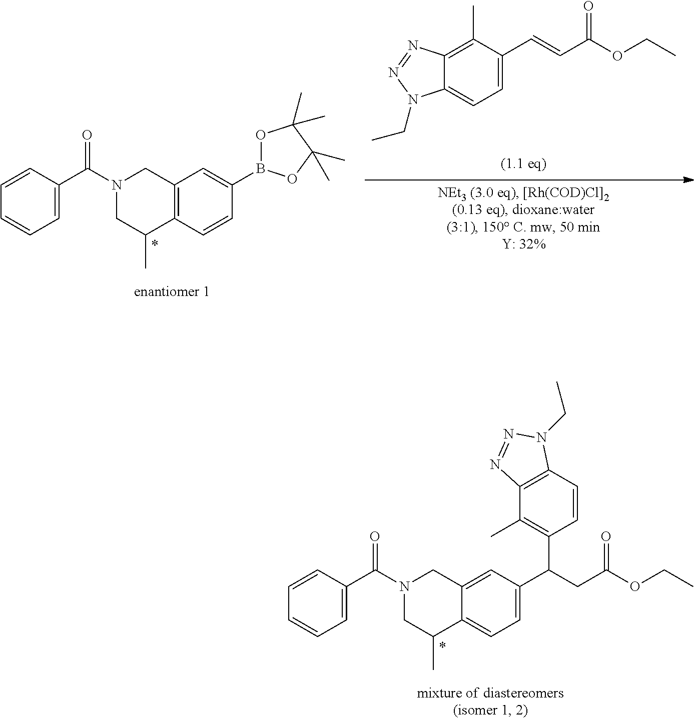

(4-Methyl-7-(4,4,5,5-tetramethyl-1,3,2-dioxaborolan-2-yl)-3,4-dihydroisoquinolin-2(1H)-yl)(phenyl)methanone enantiomer 1 (228.1 mg, 604.6 umol) and ethyl (E)-3-(1-ethyl-4-methyl-1H-benzo[d][1,2,3]triazol-5-yl)acrylate (177.2 mg, 683.2 umol, 1.1 eq) and triethylamine (251 uL, 1.81 mmol, 3.0 eq) and chloro(1,5-cyclooctadiene)rhodium(I) dimer (38.8 mg, 78.6 umol, 0.13 eq) were dissolved in dioxane (2.17 mL) and water (724 uL). After degassing, the mixture was microwaved at 150° C. for 50 min. The reaction was diluted with ethyl acetate, washed with water, washed with saturated sodium chloride, dried over magnesium sulfate, filtered, evaporated. The concentrated residue was chromatographed on silica gel (0-100% ethyl acetate in heptanes as eluent) to give ethyl 3-(2-benzoyl-4-methyl-1,2,3,4-tetrahydroisoquinolin-7-yl)-3-(1-ethyl-4-methyl-1H-benzo[d][1,2,3]triazol-5-yl)propanoate mixture of diastereomers (isomer 1,2) (99.7 mg, 32.3% yield). ESI-MS(M+H)⁺: 511.2. ¹H NMR (400 MHz, CHLOROFORM-d) d 7.29-7.46 (m, 8H), 6.97-7.20 (m, 2H), 4.83-5.04 (m, 2H), 4.47-4.81 (m, 3H), 4.03 (br. s., 2H), 3.08 (br. s., 2H), 2.86 (br. s., 3H), 1.62 (t, J=6.53 Hz, 4H), 1.23-1.39 (m, 2H), 1.03-1.23 (m, 6H).

### 11. The Preparation of 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; mixture of diastereomers (isomer 1, 2)

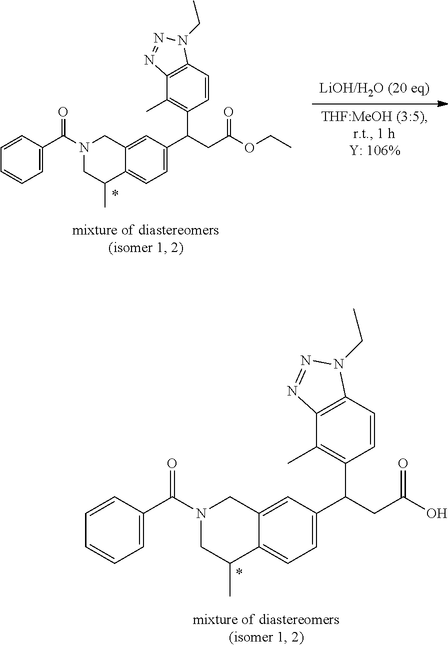 [SMILES: CCOC(=O)CC(c1ccc2c(c1)CN(C(=O)c1ccccc1)CC2C)c1ccc2c(nnn2CC)c1C ; "LiOH/H2O (20 eq)" ; "THF:MeOH (3:5),r.t., 1 hY: 106%" ; CCn1nnc2c(C)c(C(CC(=O)O)c3ccc4c(c3)CN(C(=O)c3ccccc3)CC4C)ccc21]

Ethyl 3-(2-benzoyl-4-methyl-1,2,3,4-tetrahydroisoquinolin-7-yl)-3-(1-ethyl-4-methyl-1H-benzo[d][1,2,3]triazol-5-yl)propanoate mixture of diastereomers (isomer 1,2) (99.7 mg, 195.2 umol) was dissolved in THF (1.50 mL). To this was added lithium hydroxide in water (2.6 M, 1.50 mL, 3.9 mmol, 20 eq) with stirring, followed by methanol (2.50 mL). After 1 h at RT, reaction was evaporated to dryness, partitioned between 1N HCl and ethyl acetate, washed with saturated sodium chloride, dried over magnesium sulfate, filtered and evaporated to give 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; mixture of diastereomers (isomer 1,2) (99.6 mg, 105.7% yield) ESI-MS(M+H)⁺: 483.2.

### 12. The Preparation of 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 1 [64-ent1] and 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 2 [64-ent2]

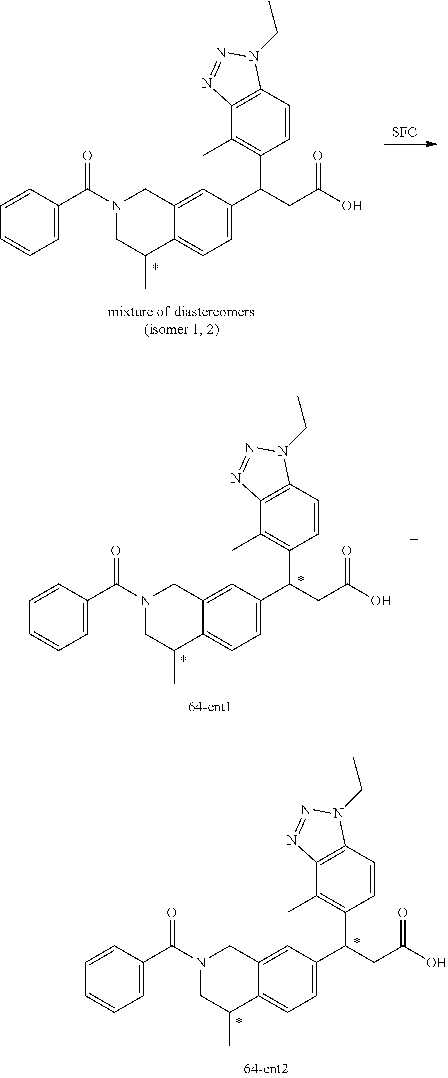 [SMILES: CCn1nnc2c(C)c(C(CC(=O)O)c3ccc4c(c3)CN(C(=O)c3ccccc3)CC4C)ccc21 ; "SFC" ; CCn1nnc2c(C)c(C(CC(=O)O)c3ccc4c(c3)CN(C(=O)c3ccccc3)CC4C)ccc21 ; CCn1nnc2c(C)c(C(CC(=O)O)c3ccc4c(c3)CN(C(=O)c3ccccc3)CC4C)ccc21]

3-(2-Benzoyl-4-methyl-1,2,3,4-tetrahydroisoquinolin-7-yl)-3-(1-ethyl-4-methyl-1H-benzo[d][1,2,3]triazol-5-yl)propanoic acid; mixture of diastereomers (isomer 1,2) (99.6 mg, 206.4 umol) was separated by chiral SFC chromatography (Column: CHIRALPAK AD-H 30×250 mm, 5 um; Co-solvent: 50% Ethanol w/0.1% DEA in CO2 (flow rate: 100 mL/min), ABPR 120 bar, MBPR 40 psi) to give the individual diastereomers. PEAK 1: 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 1 [64-ent1] mono DEA salt (45.1 mg, 38.9% yield, 100% de purity). ESI-MS(M+H)+: 483.2. 1H NMR (400 MHz, DMSO-d6) δ 7.56 (br. s., 2H), 7.37-7.50 (m, 7H), 6.90-7.23 (m, 3H), 4.43-4.89 (m, 3H), 3.21-3.77 (m, 4H), 2.90 (br. s., 3H), 2.65-2.80 (m, 3H), 1.45 (t, J=7.15 Hz, 3H), 1.01-1.24 (m, 3H). PEAK 2: 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 2 [64-ent2] mono DEA salt (46.3 mg, 40.4% yield, 100% de purity). ESI-MS(M+H)+: 483.2. 1H NMR (400 MHz, DMSO-d6) δ 7.57 (br. s., 2H), 7.37-7.50 (m, 7H), 6.91-7.23 (m, 3H), 4.45-4.88 (m, 4H), 3.19-3.80 (m, 5H), 2.91 (br. s., 2H), 2.68-2.81 (m, 3H), 1.45 (t, J=7.15 Hz, 3H), 1.01-1.26 (m, 3H).

### Example 65

### 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 3 [65-ent1] mono DEA salt and 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 4 [65-ent2] mono DEA salt

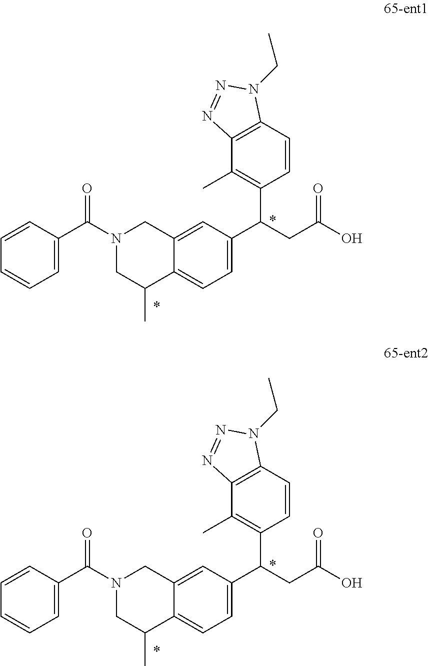 [SMILES: CCn1nnc2c(C)c(C(CC(=O)O)c3ccc4c(c3)CN(C(=O)c3ccccc3)CC4C)ccc21 ; CCn1nnc2c(C)c(C(CC(=O)O)c3ccc4c(c3)CN(C(=O)c3ccccc3)CC4C)ccc21]

3-(2-Benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 3 [65-ent1] mono DEA salt and 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 4 [65-ent2] mono DEA salt were synthesized as per Example 64 (Scheme IIIb) but utilizing the second enantiomer (PEAK 2) of the benzamide (7-bromo-4-methyl-3,4-dihydro-1H-isoquinolin-2-yl)-phenyl-methanone enantiomer 2.

Peak 1: 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 3 [65-ent1] mono DEA salt (33.0 mg, 27.8% yield, 99.7% de purity). ESI-MS(M+H)⁺: 483.2. ¹H NMR (400 MHz, DMSO-d6) δ 7.57 (br. s., 2H), 7.37-7.51 (m, 7H), 6.94-7.24 (m, 3H), 4.46-4.87 (m, 3H), 3.20-3.79 (m, 4H), 2.96 (br. s., 3H), 2.65-2.80 (m, 3H), 1.45 (t, J=7.03 Hz, 3H), 1.02-1.25 (m, 3H).

Peak 2: 3-(2-benzoyl-4-methyl-1,2,3,4-tetrahydroisoquinolin-7-yl)-3-(1-ethyl-4-methyl-1H-benzo[d][1,2,3]triazol-5-yl)propanoic acid; isomer 4 [65-ent2] mono DEA salt (34.7 mg, 28.6% yield, 99.0% de purity). ESI-MS(M+H)⁺: 483.2. ¹H NMR (400 MHz, DMSO-d6) δ 7.57 (br. s., 2H), 7.38-7.50 (m, 7H), 6.91-7.23 (m, 3H), 4.43-4.88 (m, 3H), 3.19-3.80 (m, 4H), 2.93 (br. s., 3H), 2.68-2.81 (m, 3H), 1.45 (t, J=7.15 Hz, 3H), 1.02-1.25 (m, 3H).

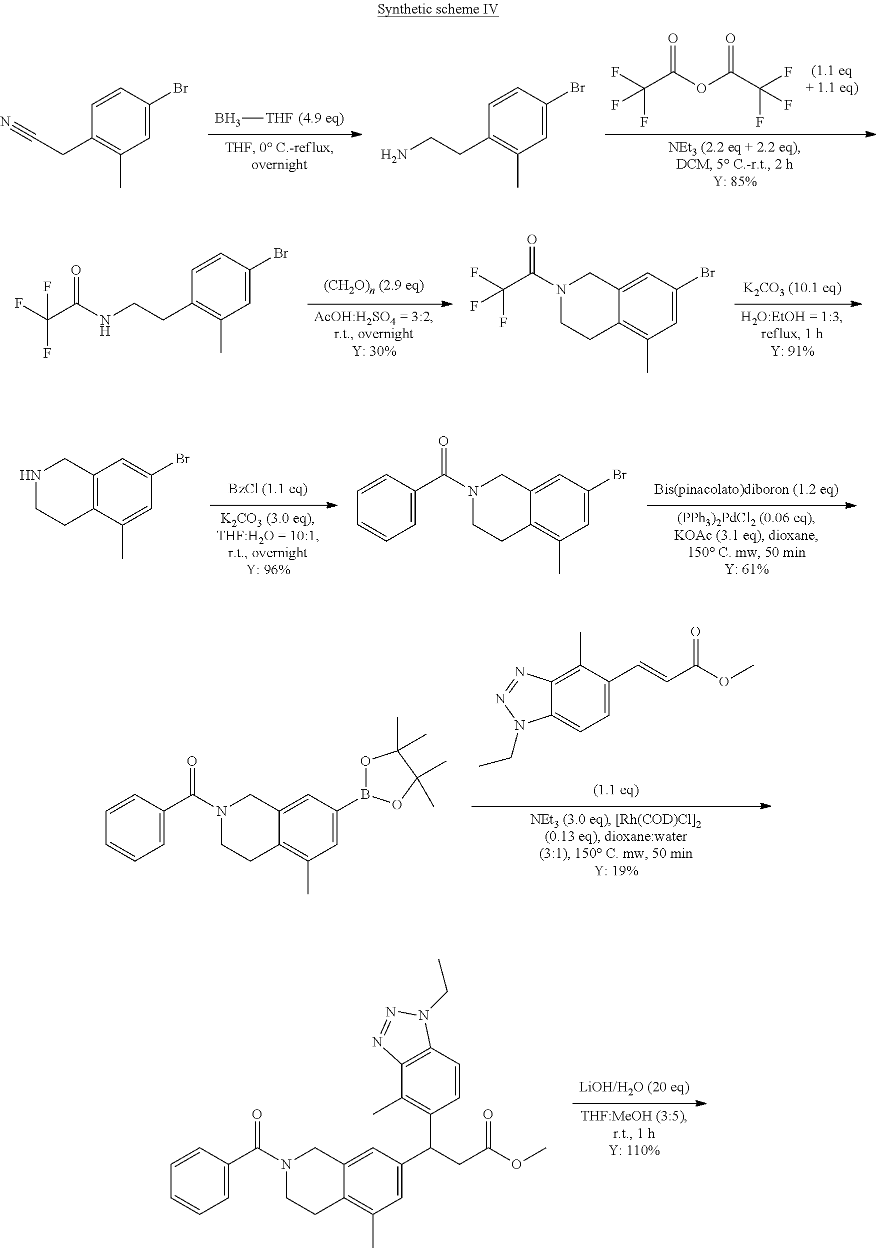

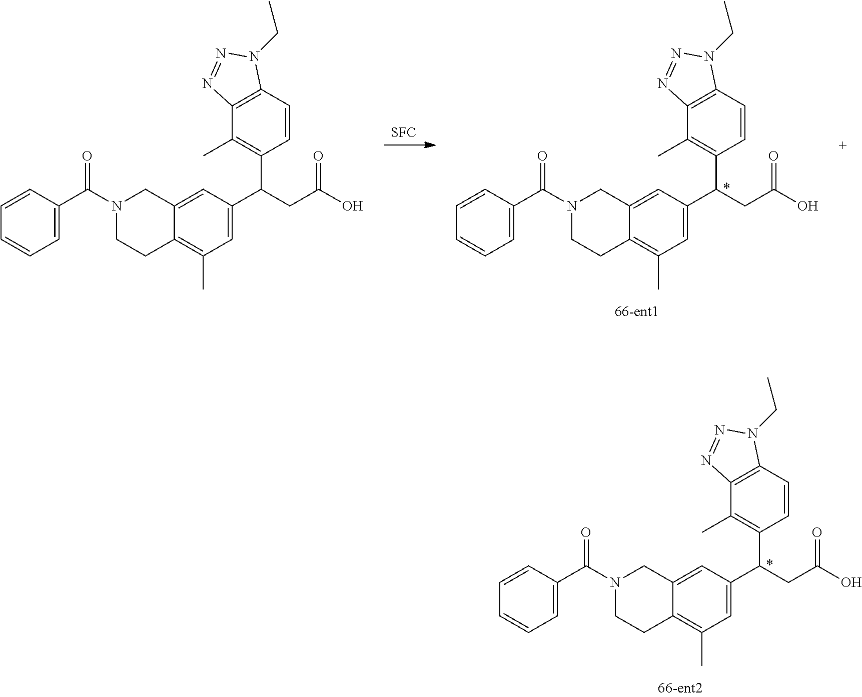 [SMILES: CCn1nnc2c(C)c(C(CC(=O)O)c3cc(C)c4c(c3)CN(C(=O)c3ccccc3)CC4)ccc21 ; "SFC" ; CCn1nnc2c(C)c(C(CC(=O)O)c3cc(C)c4c(c3)CN(C(=O)c3ccccc3)CC4)ccc21 ; CCn1nnc2c(C)c(C(CC(=O)O)c3cc(C)c4c(c3)CN(C(=O)c3ccccc3)CC4)ccc21]

### Example 66

### (S)-3-(2-benzoyl-5-methyl-1,2,3,4-tetrahydroisoquinolin-7-yl)-3-(1-ethyl-4-methyl-1H-benzo[d][1,2,3]triazol-5-yl)propanoic acid [66-ent1] and (R)-3-(2-benzoyl-5-methyl-1,2,3,4-tetrahydroisoquinolin-7-yl)-3-(1-ethyl-4-methyl-1H-benzo[d][1,2,3]triazol-5-yl)propanoic acid [66-ent2]

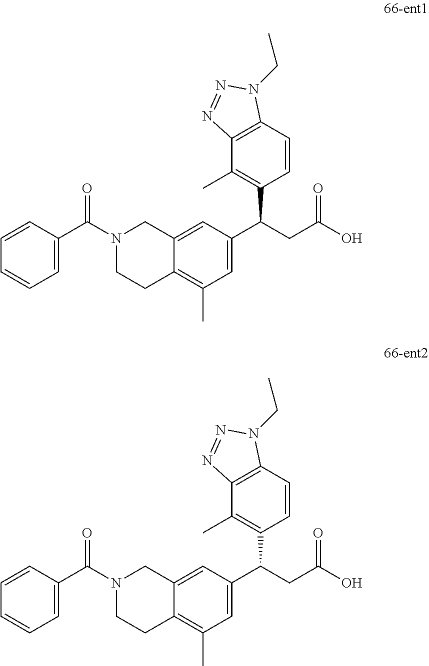 [SMILES: CCn1nnc2c(C)c([C@@H](CC(=O)O)c3cc(C)c4c(c3)CN(C(=O)c3ccccc3)CC4)ccc21 ; CCn1nnc2c(C)c([C@H](CC(=O)O)c3cc(C)c4c(c3)CN(C(=O)c3ccccc3)CC4)ccc21]

### 1. The Preparation of 2-(4-bromo-2-methyl-phenyl)ethanamine

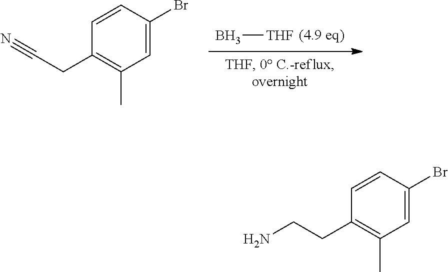

To 2-(4-bromo-2-methyl-phenyl)acetonitrile (1.00 g, 4.76 mmol) in THF (12.7 mL) was added borane-THF (1 M in THF, 23.5 mL, 23.5 mmol, 4.9 eq) at 0° C. The reaction was heated to reflux overnight. 4M HCl in dioxane was added to pH 2. Evaporation was followed by dissolution in methanol and evaporation (twice) to give 2-(4-bromo-2-methyl-phenyl)ethanamine. ESI-MS(M+H)⁺: 214.0/216.0. ¹H NMR (400 MHz, METHANOL-d4) δ 7.23-7.41 (m, 2H), 7.10 (d, J=8.28 Hz, 1H), 2.88-3.15 (m, 4H), 2.34 (br. s., 3H)

### 2. The Preparation of N-[2-(4-bromo-2-methyl-phenyl)ethyl]-2,2,2-trifluoro-acetamide

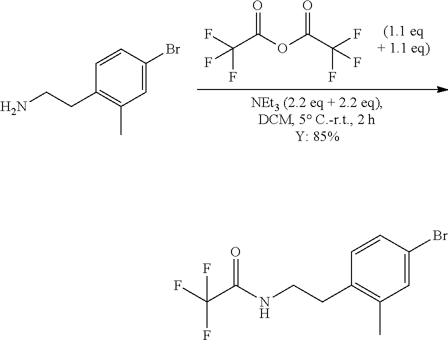

To a solution of 2-(4-bromo-2-methyl-phenyl)ethanamine (1.02 g, 4.76 mmol) and triethylamine (1.44 mL, 10.42 mmol, 2.2 eq) in DCM (18.1 mL) was added trifluoroacetic acid; anhydride (722 uL, 5.19 mmol, 1.1 eq) at 5° C. dropwise and with vigorous stirring. After 90 min, additional trifluoroacetic acid; anhydride (722 uL, 5.19 mmol, 1.1 eq) and triethylamine (1.44 mL, 10.42 mmol, 2.2 eq) were added. After additional 30 min, the reaction was poured into ice-water and extracted with DCM. The organic layer was washed with water, dried over anhydrous magnesium sulfate, filtered and concentrated in vacuo. Purification by preparative HPLC gave N-[2-(4-bromo-2-methyl-phenyl)ethyl]-2,2,2-trifluoro-acetamide (1.26 g, 85.4% yield). ESI-MS(M+H)⁺: 310.0. ¹H NMR (400 MHz, CHLOROFORM-d) δ 7.35 (s, 1H), 7.30 (dd, J=1.76, 8.28 Hz, 1H), 6.98 (d, J=8.28 Hz, 1H), 6.30 (br. s., 1H), 3.57 (q, J=6.78 Hz, 2H), 2.87 (t, J=7.28 Hz, 2H), 2.33 (s, 3H).

### 3. The Preparation of 1-(7-bromo-5-methyl-3,4-dihydro-1H-isoquinolin-2-yl)-2,2,2-trifluoro-ethanone

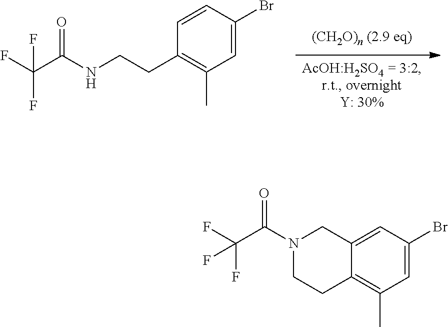

N-[2-(4-bromo-2-methyl-phenyl)ethyl]-2,2,2-trifluoro-acetamide (1.26 g, 4.06 mmol) and paraformaldehyde (366.04 mg, 12.19 mmol, 3.0 eq) were dissolved in premixed solution of acetic acid; (6.71 mL) and sulfuric acid; (4.47 mL). The reaction was stirred overnight. The reaction was poured into cold water, which was extracted with ethyl acetate. The organic layer was washed with saturated sodium bicarbonate, then with water, then with saturated sodium chloride. The solution was dried over magnesium sulfate, filtered and evaporated. Sample was purified by silica gel chromatography using 0-100% ethyl acetate in heptanes as eluent to yield 1-(7-bromo-5-methyl-3,4-dihydro-1H-isoquinolin-2-yl)-2,2,2-trifluoro-ethanone (398.7 mg, 30.5% yield). ESI-MS(M+H)⁺: 322.0. ¹H NMR (400 MHz, CHLOROFORM-d) δ 7.24 (s, 1H), 7.11-7.18 (m, 1H), 4.68-4.78 (m, 2H), 3.83-3.95 (m, 2H), 2.78 (q, J=5.94 Hz, 2H), 2.21-2.26 (m, 3H).

### 4. The Preparation of 7-bromo-5-methyl-1,2,3,4-tetrahydroisoquinoline

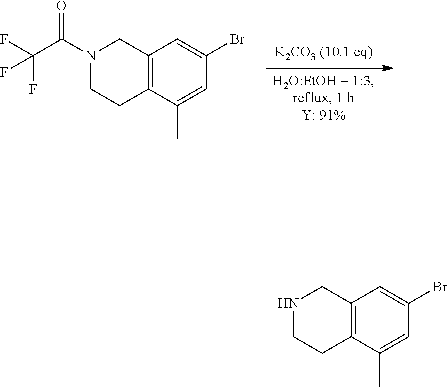

1-(7-Bromo-5-methyl-3,4-dihydro-1H-isoquinolin-2-yl)-2,2,2-trifluoro-ethanone (398.7 mg, 1.24 mmol) was dissolved in ethyl alcohol (12.9 mL) and a solution of potassium carbonate (1.72 g, 12.44 mmol, 10 eq) in water (4.30 mL) was added. The mixture was heated to reflux for 1 hour. The reaction was cooled and evaporated in vacuo. Water was added to the residue and the mixture was extracted with DCM (3×). The combined DCM layers were washed with water, dried over MgSO4, filtered and concentrated in vacuo to give the product 7-bromo-5-methyl-1,2,3,4-tetrahydroisoquinoline (254.2 mg, 90.7% yield). ESI-MS(M+H)⁺: 226.0.

### 5. The Preparation of (7-bromo-5-methyl-3,4-dihydro-1H-isoquinolin-2-yl)-phenyl-methanone

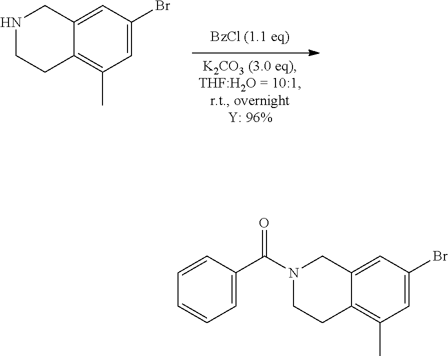

7-Bromo-5-methyl-1,2,3,4-tetrahydroisoquinoline (254.2 mg, 1.12 mmol) and potassium carbonate (466.1 mg, 3.37 mmol, 3.0 eq) were slurried in THF (3.11 mL) and water (311 uL) and to this was added benzoyl chloride (144 uL, 1.24 mmol, 1.1 eq) and the reaction was stirred at RT for 30 min. The reaction was diluted with ethyl acetate and washed with water, then with brine. The organics were dried with MgSO₄, filtered and evaporated. The residue was purified with silica gel chromatography using 0-50% ethyl acetate in heptanes as eluent to give (7-bromo-5-methyl-3,4-dihydro-1H-isoquinolin-2-yl)-phenyl-methanone (353.8 mg, 95.7% yield). ESI-MS(M+H)⁺: 330.1.

### 6. The Preparation of (5-methyl-7-(4,4,5,5-tetramethyl-1,3,2-dioxaborolan-2-yl)-3,4-dihydroisoquinolin-2(1H)-yl)(phenyl)methanone

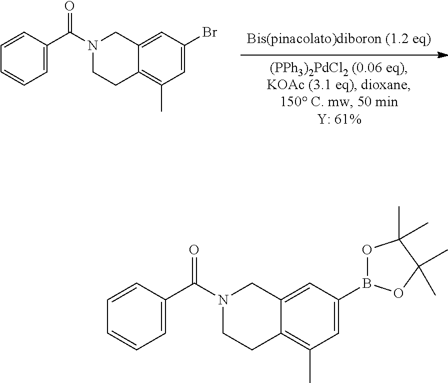 [SMILES: Cc1cc(Br)cc2c1CCN(C(=O)c1ccccc1)C2 ; "(PPh3)2PdCl2 (0.06 eq),KOAc (3.1 eq), dioxane,150° C. mw, 50 minY: 61%" ; Cc1cc(B2OC(C)(C)C(C)(C)O2)cc2c1CCN(C(=O)c1ccccc1)C2]

(7-Bromo-5-methyl-3,4-dihydro-1H-isoquinolin-2-yl)-phenyl-methanone (353.8 mg, 1.07 mmol) and bis(pinacolato)diboron (334.6 mg, 1.32 mmol, 1.2 eq) and bis(triphenylphosphine)palladium(II) dichloride (45.1 mg, 64.3 umol, 0.06 eq) and potassium acetate (323.9 mg, 3.30 mmol, 3.1 eq) were dissolved in dioxane (3.85 mL). After degassing, the reaction was sealed and microwaved at 150° C. for 50 min. The reaction was diluted with ethyl acetate, washed with water, washed with saturated sodium chloride, dried over magnesium sulfate, filtered. After evaporation, the residue was chromatographed on silica gel (0-100% ethyl acetate in heptanes as eluent) to give (5-methyl-7-(4,4,5,5-tetramethyl-1,3,2-dioxaborolan-2-yl)-3,4-dihydroisoquinolin-2(1H)-yl)(phenyl)methanone (247.4 mg, 61.3% yield). ESI-MS(M+H)⁺: 378.2. ¹H NMR (400 MHz, CHLOROFORM-d) δ 7.39-7.54 (m, 7H), 4.92 (br. s., 1H), 4.62 (br. s., 1H), 4.03 (br. s., 1H), 3.67 (br. s., 1H), 2.70-2.94 (m, 2H), 2.27 (s, 3H), 1.23-1.41 (m, 12H).

### 7. The Preparation of methyl 3-(2-benzoyl-5-methyl-1,2,3,4-tetrahydroisoquinolin-7-yl)-3-(1-ethyl-4-methyl-1H-benzo[d][1,2,3]triazol-5-yl)propanoate

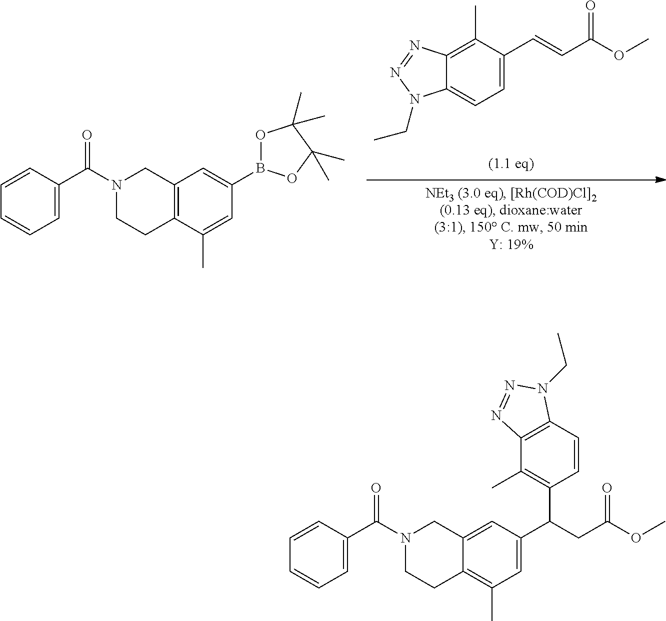

(5-Methyl-7-(4,4,5,5-tetramethyl-1,3,2-dioxaborolan-2-yl)-3,4-dihydroisoquinolin-2(1H)-yl)(phenyl)methanone (247.4 mg, 655.8 umol) and methyl (E)-3-(1-ethyl-4-methyl-1H-benzo[d][1,2,3]triazol-5-yl)acrylate (181.8 mg, 741.0 umol, 1.1 eq) and triethylamine (273 uL, 1.97 mmol, 3.0 eq) and chlororhodium;(1Z,5Z)-cycloocta-1,5-diene (42.0 mg, 85.2 umol, 0.13 eq) were dissolved in dioxane (2.36 mL) and water (785 uL). After degassing, the mixture was microwaved at 150° C. for 50 min. The reaction was diluted with ethyl acetate, washed with water, washed with saturated sodium chloride, dried over magnesium sulfate, filtered, evaporated. The residue was chromatographed on silica gel (0-100% ethyl acetate in heptanes as eluent) to give methyl 3-(2-benzoyl-5-methyl-1,2,3,4-tetrahydroisoquinolin-7-yl)-3-(1-ethyl-4-methyl-1H-benzo[d][1,2,3]triazol-5-yl)propanoate (61.0 mg, 18.7% yield). ESI-MS(M+H)⁺: 497.3. ¹H NMR (400 MHz, CHLOROFORM-d) δ 7.29-7.49 (m, 8H), 6.83-7.02 (m, 1H), 4.50-4.98 (m, 3H), 3.52-3.65 (m, 3H), 2.88 (br. s., 3H), 2.20 (s, 3H), 1.63 (t, J=7.15 Hz, 3H).

### 8. The Preparation of 3-(2-benzoyl-5-methyl-3,4-dihydro-1H-isoquinolin-7-yl)-3-(1-ethyl-4-methyl-benzotriazol-5-yl)propanoic acid;

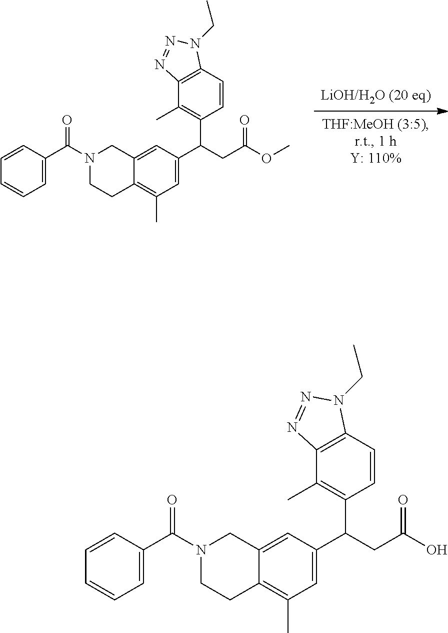 [SMILES: CCn1nnc2c(C)c(C(CC(=O)OC)c3cc(C)c4c(c3)CN(C(=O)c3ccccc3)CC4)ccc21 ; "THF:MeOH (3:5),r.t., 1 hY: 110%" ; CCn1nnc2c(C)c(C(CC(=O)O)c3cc(C)c4c(c3)CN(C(=O)c3ccccc3)CC4)ccc21]

Methyl 3-(2-benzoyl-5-methyl-3,4-dihydro-1H-isoquinolin-7-yl)-3-(1-ethyl-4-methyl-benzotriazol-5-yl)propanoate (61.00 mg, 122.84 umol) was dissolved in THF (944 uL). To this was added lithium hydroxide in water (2.6 M, 945 uL, 2.46 mmol, 20 eq) with stirring, followed by methanol (1.57 mL). After 1 h at RT, reaction was evaporated to dryness, partitioned between 1N HCl, ethyl acetate. Organics were washed with saturated sodium chloride, dried over magnesium sulfate, filtered and evaporated to give the product 3-(2-benzoyl-5-methyl-3,4-dihydro-1H-isoquinolin-7-yl)-3-(1-ethyl-4-methyl-benzotriazol-5-yl)propanoic acid; (65.2 mg, 110% yield) as mixture of enantiomers. ESI-MS(M+H)⁺: 483.2.

### 9. The Preparation of 3-(2-benzoyl-5-methyl-3,4-dihydro-1H-isoquinolin-7-yl)-3-(1-ethyl-4-methyl-benzotriazol-5-yl)propanoic acid; enantiomer 1 [66-ent1] mono DEA salt and 3-(2-benzoyl-5-methyl-3,4-dihydro-1H-isoquinolin-7-yl)-3-(1-ethyl-4-methyl-benzotriazol-5-yl)propanoic acid; enantiomer 2 [66-ent2] mono DEA salt

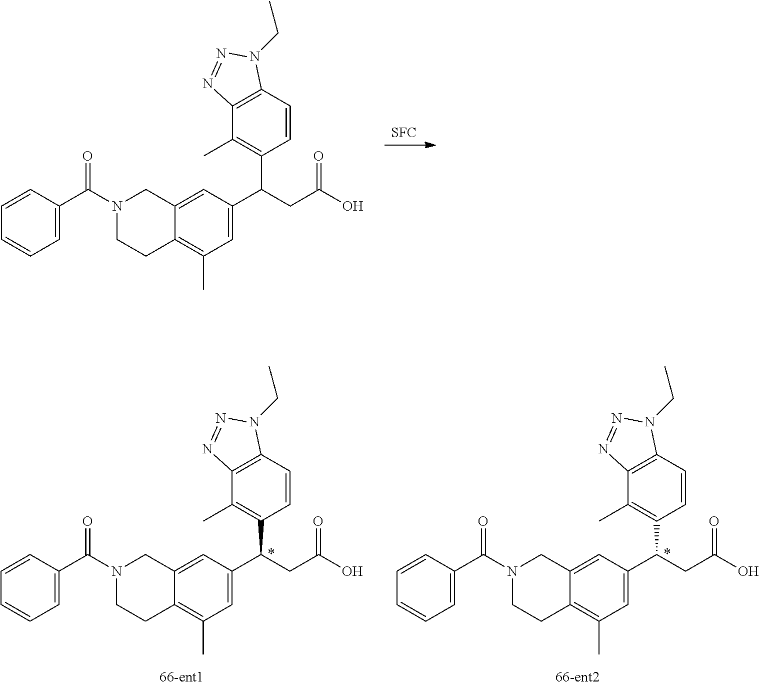 [SMILES: CCn1nnc2c(C)c(C(CC(=O)O)c3cc(C)c4c(c3)CN(C(=O)c3ccccc3)CC4)ccc21 ; "SFC" ; CCn1nnc2c(C)c([C@@H](CC(=O)O)c3cc(C)c4c(c3)CN(C(=O)c3ccccc3)CC4)ccc21 ; CCn1nnc2c(C)c([C@H](CC(=O)O)c3cc(C)c4c(c3)CN(C(=O)c3ccccc3)CC4)ccc21]

3-(2-Benzoyl-5-methyl-3,4-dihydro-1H-isoquinolin-7-yl)-3-(1-ethyl-4-methyl-benzotriazol-5-yl)propanoic acid; (65.20 mg, 135.11 umol) was separated by chiral SFC (Column: CHIRALPAK AD-H 30×250 mm, 5 um; Co-solvent: 50% Ethanol w/0.1% DEA in CO₂ (flow rate: 100 mL/min), ABPR 120 bar, MBPR 40 psi) to give the two individual enantiomers. PEAK 1: (S)-3-(2-benzoyl-5-methyl-1,2,3,4-tetrahydroisoquinolin-7-yl)-3-(1-ethyl-4-methyl-1H-benzo[d][1,2,3]triazol-5-yl)propanoic acid; enantiomer 1 [66-ent1] mono DEA salt: (23.2 mg, 30.9% yield, 100% ee purity). ESI-MS(M+H)⁺: 483.2. ¹H NMR (400 MHz, DMSO-d6) δ 7.38-7.62 (m, 7H), 7.00 (s, 2H), 4.45-4.83 (m, 3H), 3.83 (br. s., 1H), 3.54 (br. s., 3H), 2.96 (br. s., 1H), 2.62-2.80 (m, 6H), 2.13 (s, 3H), 1.46 (t, J=7.03 Hz, 3H). PEAK 2: (R)-3-(2-benzoyl-5-methyl-1,2,3,4-tetrahydroisoquinolin-7-yl)-3-(1-ethyl-4-methyl-1H-benzo[d][1,2,3]triazol-5-yl)propanoic acid; enantiomer 2 [66-ent2] mono DEA salt (24.8 mg, 33.0% yield, 100% ee purity). ESI-MS(M+H)⁺: 483.2. ¹H NMR (400 MHz, DMSO-d6) δ 7.38-7.63 (m, 7H), 6.96-7.07 (m, 2H), 4.45-4.82 (m, 3H), 3.84 (br. s., 1H), 3.53 (br. s., 3H), 2.95 (br. s., 1H), 2.62-2.79 (m, 6H), 2.13 (s, 3H), 1.46 (t, J=7.15 Hz, 3H). The absolute configuration of 66-Ent1 was determined by an X-ray co-crystal structure with the KELCH domain of KEAP1.

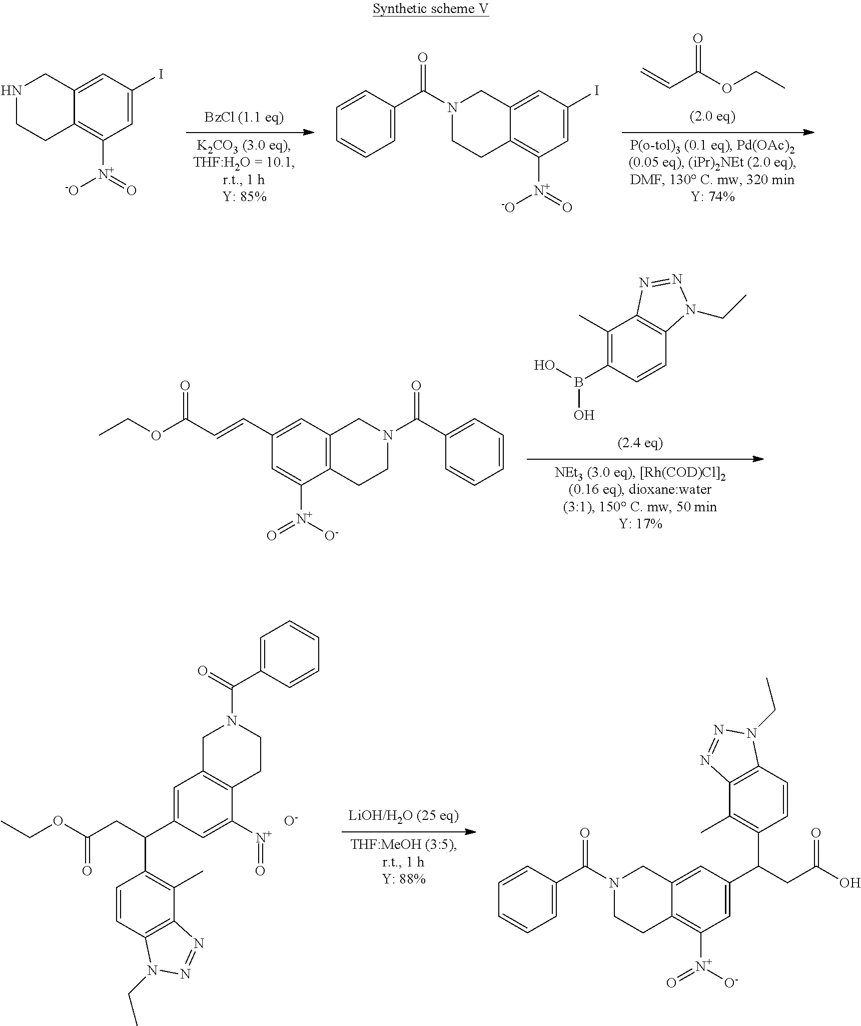

### Example 67

### 3-(2-benzoyl-5-nitro-3,4-dihydro-1H-isoquinolin-7-yl)-3-(1-ethyl-4-methyl-benzotriazol-5-yl)propanoic acid; [67]

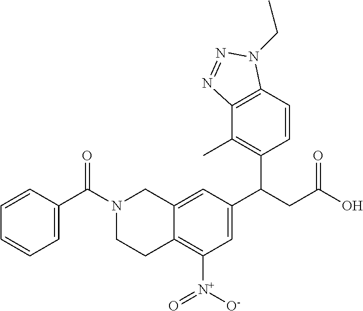 [SMILES: CCn1nnc2c(C)c(C(CC(=O)O)c3cc4c(c([N+](=O)[O-])c3)CCN(C(=O)c3ccccc3)C4)ccc21]

### 1. The Preparation of (7-iodo-5-nitro-3,4-dihydro-1H-isoquinolin-2-yl)-phenyl-methanone

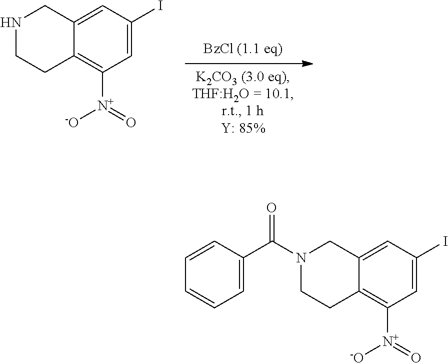

7-Iodo-5-nitro-1,2,3,4-tetrahydroisoquinoline (352.2 mg, 1.16 mmol) and potassium carbonate (480.2 mg, 3.47 mmol, 3.0 eq) were slurried in THF (3.20 mL) and water (320 uL) and to this was added benzoyl chloride (148 uL, 1.27 mmol, 1.1 eq) and the reaction was stirred at RT for one hour. The reaction was diluted with ethyl acetate and washed with water, then with brine. The organics were dried with MgSO₄, filtered and evaporated. The residue was purified with silica gel chromatography (0-50% ethyl acetate in heptanes as eluent) to give (7-iodo-5-nitro-3,4-dihydro-1H-isoquinolin-2-yl)-phenyl-methanone (404.3 mg, 85.4% yield). ESI-MS(M+H)⁺: 409.0. ¹H NMR (400 MHz, CHLOROFORM-d) δ 8.18 (s, 1H), 7.36-7.88 (m, 6H), 4.54-5.00 (m, 2H), 3.57-4.16 (m, 2H), 2.99-3.27 (m, 2H).

### 2. The Preparation of (E)-3-(2-benzoyl-5-nitro-3,4-dihydro-1H-isoquinolin-7-yl)prop-2-enoate

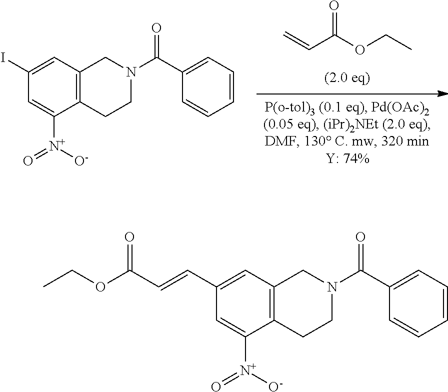

To a mixture of (7-iodo-5-nitro-3,4-dihydro-1H-isoquinolin-2-yl)-phenyl-methanone (101.7 mg, 249.2 umol) and ethyl acrylate (54 uL, 496 umol, 2.0 eq) and tri(o-tolyl)phosphine (15.2 mg, 49.8 umol, 0.1 eq) and palladium(II) acetate (5.6 mg, 24.9 umol, 0.05 eq) in DMF (1.00 mL) was added diisopropylethylamine (87 uL, 496 umol, 2.0 eq). The mixture was heated with microwave irritation at 130° C. for 320 min. The reaction was diluted with water, extracted with ethyl acetate, washed with saturated sodium chloride, dried over magnesium sulfate, filtered and evaporated. Sample was purified by silica gel chromatography using 0-30% ethyl acetate in heptanes as eluent to give ethyl (E)-3-(2-benzoyl-5-nitro-3,4-dihydro-1H-isoquinolin-7-yl)prop-2-enoate (70.0 mg, 73.9% yield). ESI-MS(M+H)⁺: 381.1. ¹H NMR (400 MHz, CHLOROFORM-d) δ 8.04 (s, 1H), 7.56-7.69 (m, 2H), 7.43-7.53 (m, 5H), 6.45-6.56 (m, 1H), 4.98 (br. s., 2H), 4.29 (q, J=7.03 Hz, 2H), 3.70 (br. s., 2H), 3.21 (br. s., 2H), 1.36 (t, J=7.03 Hz, 3H).

### 3. The Preparation of (1-ethyl-4-methyl-benzotriazol-5-yl)boronic acid

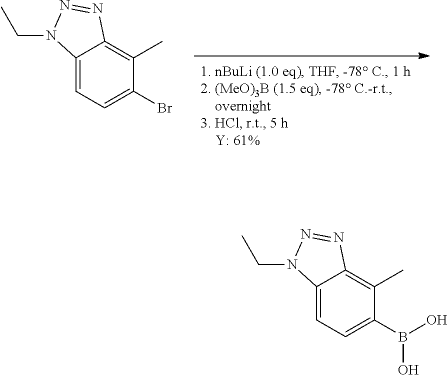

To a solution of 5-bromo-1-ethyl-4-methyl-benzotriazole (5.00 g, 20.8 mmol) in THF (100 mL) at −78° C. was added butyllithium (2.5 M in hexanes, 8.33 mL, 20.8 mmol, 1.0 eq) dropwise. After 1 h, trimethyl borate (3.49 mL, 31.23 mmol, 1.5 eq) was added dropwise. The solution was allowed to warm to RT and stir overnight. The reaction was quenched with 1 N HCl and allowed to stir for 5 hours. The reaction pH was adjusted to 3 using saturated sodium bicarbonate solution and then the mixture was diluted with brine and extracted with ethyl acetate. The combined organic layers were washed with brine, dried over MgSO₄, filtered, and concentrated. Trituration with diethyl ether afforded (1-ethyl-4-methyl-benzotriazol-5-yl)boronic acid; (2.60 g, 60.9% yield). ESI-MS(M+H)⁺: 206.1. ¹H NMR (400 MHz, CHLOROFORM-d) δ 8.36 (d, J=8.53 Hz, 1H), 7.51 (d, J=8.53 Hz, 1H), 4.75 (q, J=7.45 Hz, 2H), 3.39 (s, 3H), 1.69 (t, J=7.28 Hz, 3H).

### 4. The Preparation of ethyl 3-(2-benzoyl-5-nitro-3,4-dihydro-1H-isoquinolin-7-yl)-3-(1-ethyl-4-methyl-benzotriazol-5-yl)propanoate

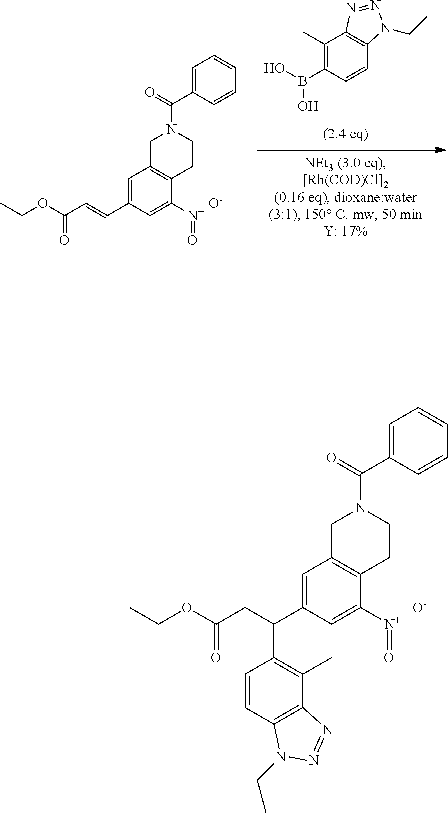

To a solution of ethyl (E)-3-(2-benzoyl-5-nitro-3,4-dihydro-1H-isoquinolin-7-yl)prop-2-enoate (70.0 mg, 184.0 umol) in dioxane (2.00 mL) and water (667 uL) was added (1-ethyl-4-methyl-benzotriazol-5-yl)boronic acid; (89.0 mg, 434 umol, 2.4 eq) and triethylamine (77 uL, 552 umol, 3.0 eq) and then chloro(1,5-cyclooctadiene)rhodium(I) dimer (14.5 mg, 29.4 umol, 0.16 eq). The resulting mixture was degassed and then was microwaved at 150° C. for 50 min. The reaction mixture was diluted with water, extracted with ethyl acetate (3×), washed with saturated sodium chloride, dried over magnesium sulfate, filtered, evaporated. Purification by silica gel chromatography (0-100% ethyl acetate in heptanes as eluent) gave the product ethyl 3-(2-benzoyl-5-nitro-3,4-dihydro-1H-isoquinolin-7-yl)-3-(1-ethyl-4-methyl-benzotriazol-5-yl)propanoate (16.7 mg, 16.8% yield). ESI-MS(M+H)⁺: 542.2. ¹H NMR (400 MHz, CHLOROFORM-d) δ 7.78 (s, 1H), 7.45 (s, 6H), 7.29-7.41 (m, 2H), 4.84-5.09 (m, 1H), 4.67 (q, J=7.11 Hz, 2H), 4.03-4.11 (m, 2H), 3.13 (br. s., 4H), 2.85 (s, 3H), 1.63 (t, J=7.28 Hz, 3H), 1.16 (t, J=6.78 Hz, 3H).

### 5. The Preparation of 3-(2-benzoyl-5-nitro-3,4-dihydro-1H-isoquinolin-7-yl)-3-(1-ethyl-4-methyl-benzotriazol-5-yl)propanoic acid; [67]

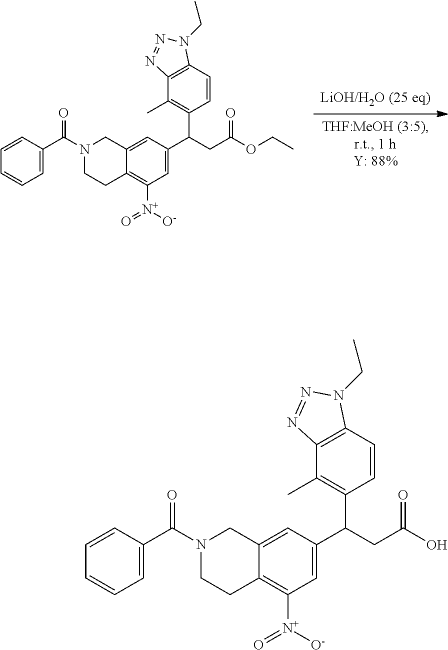 [SMILES: CCOC(=O)CC(c1cc2c(c([N+](=O)[O-])c1)CCN(C(=O)c1ccccc1)C2)c1ccc2c(nnn2CC)c1C ; "THF:MeOH (3:5),r.t., 1 hY: 88%" ; CCn1nnc2c(C)c(C(CC(=O)O)c3cc4c(c([N+](=O)[O-])c3)CCN(C(=O)c3ccccc3)C4)ccc21]

Ethyl 3-(2-benzoyl-5-nitro-3,4-dihydro-1H-isoquinolin-7-yl)-3-(1-ethyl-4-methyl-benzotriazol-5-yl)propanoate (16.7 mg, 30.8 umol) was dissolved in THF (300 uL). To this was added lithium hydroxide in water (2.6 M, 300.00 uL, 780 umol, 25 eq) with stirring, followed by methanol (500 uL). After 1 h at RT, reaction was evaporated to dryness, partitioned between 1N HCl, ethyl acetate. The organics were washed with saturated sodium chloride, dried over sodium sulfate, filtered, evaporated to give the product 3-(2-benzoyl-5-nitro-3,4-dihydro-1H-isoquinolin-7-yl)-3-(1-ethyl-4-methyl-benzotriazol-5-yl)propanoic acid; [67] (14.0 mg, 88.4% yield, 100% purity) as mixture of enantiomers. ESI-MS(M+H)⁺: 514.2. ¹H NMR (400 MHz, DMSO-d6) δ 11.99-12.36 (m, 1H), 7.69-7.86 (m, 2H), 7.46 (d, J=1.00 Hz, 7H), 4.75-4.99 (m, 3H), 4.59-4.73 (m, 2H), 3.44-3.59 (m, 2H), 3.01-3.30 (m, 2H), 2.92-3.01 (m, 2H), 2.71-2.86 (m, 3H), 1.40-1.52 (m, 3H).

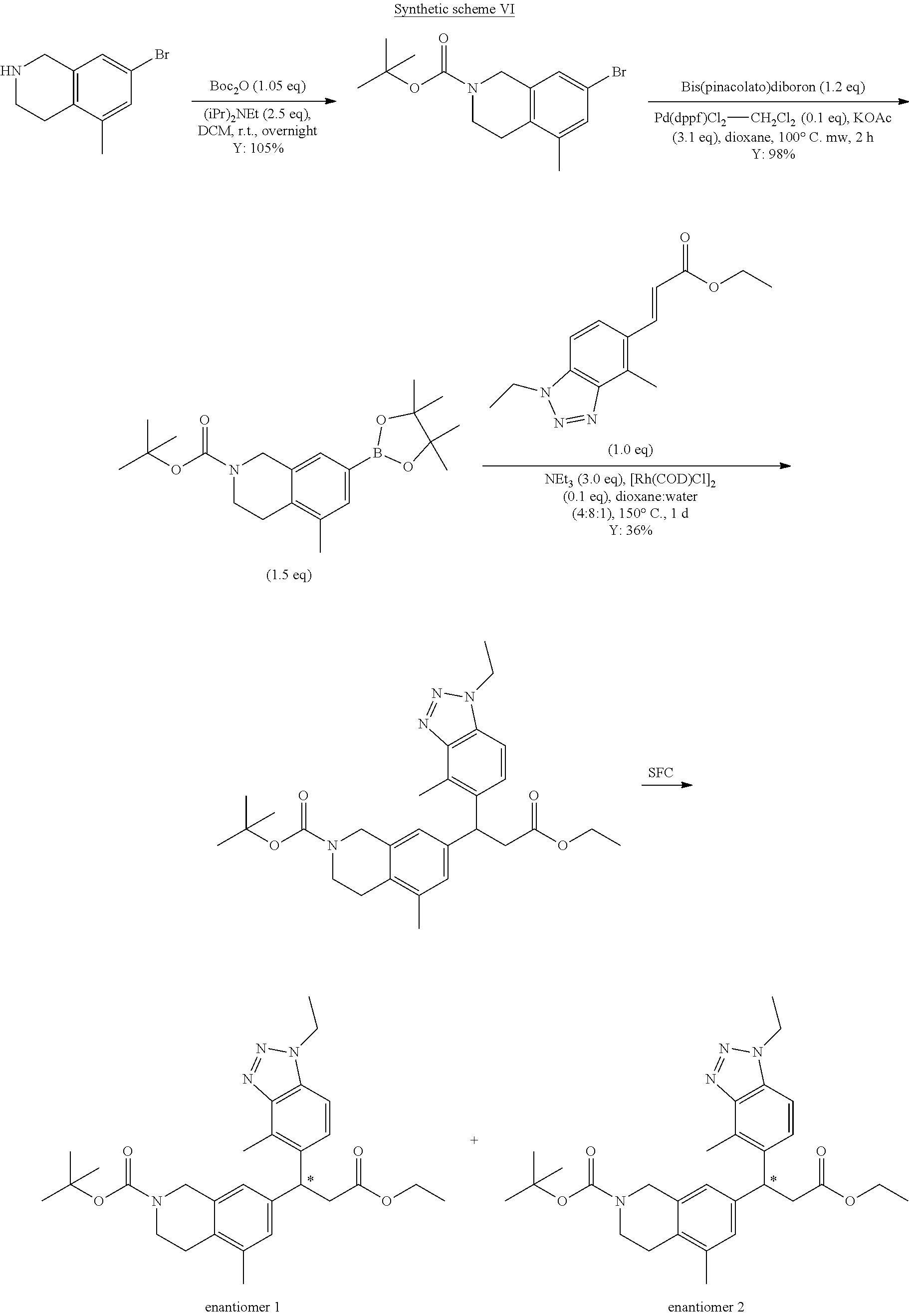

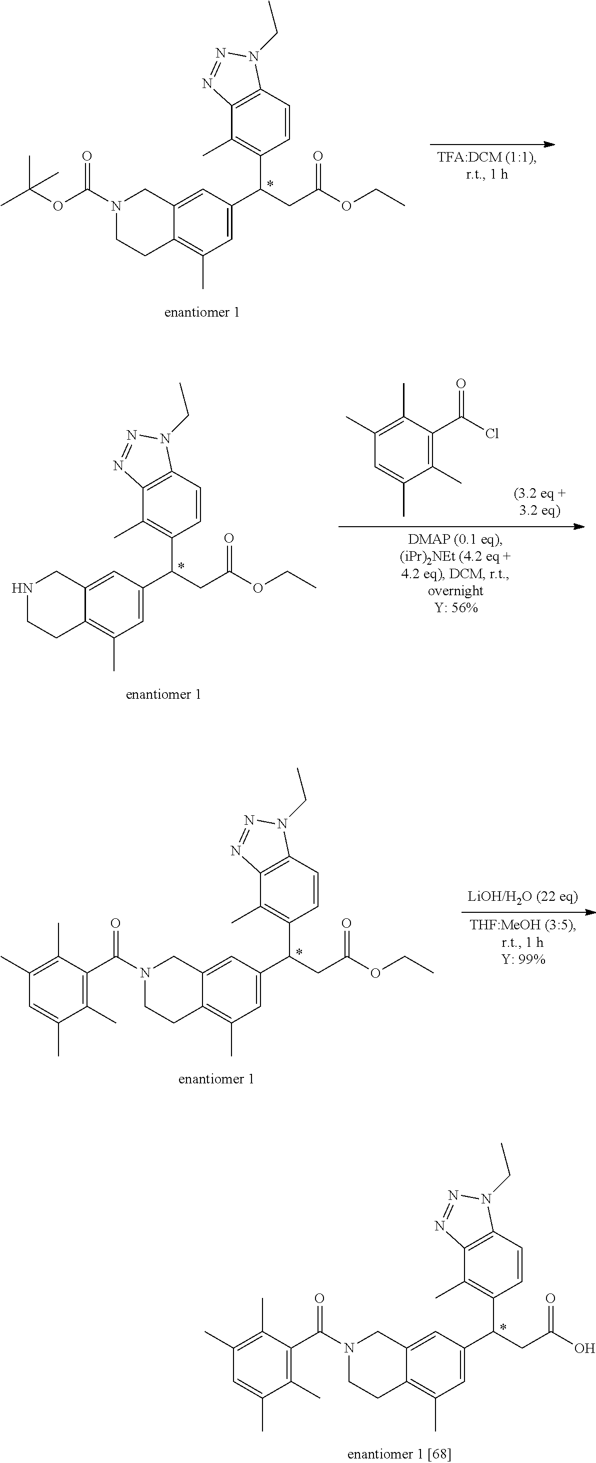

### Example 68

### 3-(1-ethyl-4-methyl-benzotriazol-5-yl)-3-[5-methyl-2-(2,3,5,6-tetramethylbenzoyl)-3,4-dihydro-1H-isoquinolin-7-yl]propanoic acid; enantiomer 1 [68]

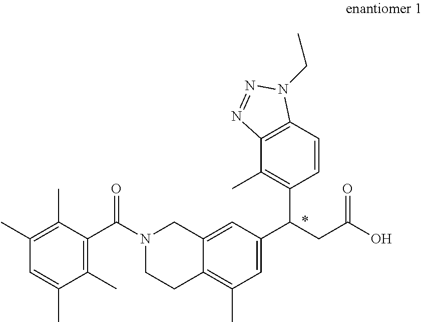 [SMILES: CCn1nnc2c(C)c(C(CC(=O)O)c3cc(C)c4c(c3)CN(C(=O)c3c(C)c(C)cc(C)c3C)CC4)ccc21]

### 1. Preparation of tert-butyl 7-bromo-5-methyl-3,4-dihydro-1H-isoquinoline-2-carboxylate

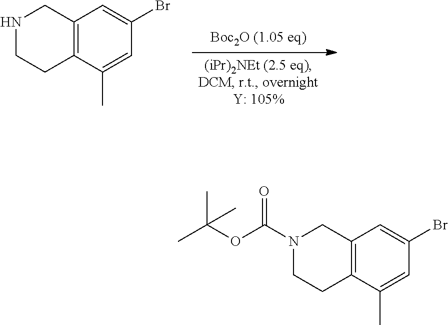 [SMILES: Cc1cc(Br)cc2c1CCNC2 ; "(iPr)2NEt (2.5 eq),DCM, r.t., overnightY: 105%" ; "Boc2O (1.05 eq)" ; Cc1cc(Br)cc2c1CCN(C(=O)OC(C)(C)C)C2]

7-Bromo-5-methyl-1,2,3,4-tetrahydroisoquinoline (2.11 g, 9.33 mmol) and di-tert-butyl-dicarbonate (2.14 g, 9.80 mmol, 1.05 eq) were dissolved in DCM (100 mL). To this was added diisopropylethylamine (4.07 mL, 23.33 mmol, 2.5 eq) dropwise and the reaction was stirred at room temperature overnight. The reaction was diluted with water, extracted with DCM, washed with water, dried over magnesium sulfate, filtered, evaporated to give the product tert-butyl 7-bromo-5-methyl-3,4-dihydro-1H-isoquinoline-2-carboxylate (3.21 g, 105.5% yield). ESI-MS(2M+Na)⁺: 673.0/675.1. ¹H NMR (400 MHz, CHLOROFORM-d) δ 7.19 (s, 1H), 7.11 (s, 1H), 4.54 (s, 2H), 3.66 (t, J=5.77 Hz, 2H), 2.66 (t, J=5.77 Hz, 2H), 2.22 (s, 3H), 1.49 (s, 9H).

### 2. The Preparation of tert-butyl 5-methyl-7-(4,4,5,5-tetramethyl-1,3,2-dioxaborolan-2-yl)-3,4-dihydro-1H-isoquinoline-2-carboxylate

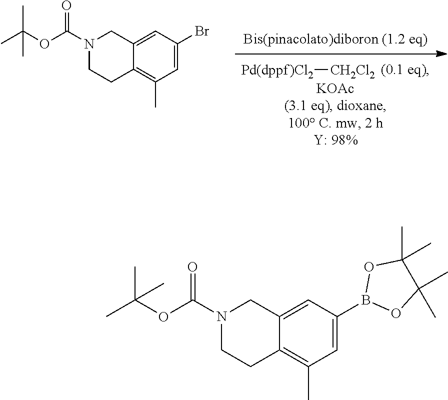

Tert-butyl 7-bromo-5-methyl-3,4-dihydro-1H-isoquinoline-2-carboxylate (1.10 g, 3.38 mmol) and bis(pinacolato)diboron (1053 mg, 4.15 mmol, 1.2 eq) and bis(diphenylphosphino)ferrocene]palladium(II) dichloride complex with dichloromethane (275 mg, 337 umol, 0.1 eq) and potassium acetate (1019 mg, 10.38 mmol, 3.1 eq) were dissolved in dioxane (12.1 mL). After degassing, the reaction was sealed (in two vials) and heated at 100° C. for 2 hours. Reactions combined, diluted with aqueous sodium chloride, extracted with ethyl acetate. Organic layers washed with saturated sodium chloride, dried over magnesium sulfate, filtered, evaporated. Residue purified by silica gel chromatography using 0-100% ethyl acetate in heptanes as eluent to yield the product tert-butyl 5-methyl-7-(4,4,5,5-tetramethyl-1,3,2-dioxaborolan-2-yl)-3,4-dihydro-1H-isoquinoline-2-carboxylate (1.72 g, 98% yield). Note that sample contains 28% solvent by mass. ESI-MS(M+Na)⁺: 396.2. ¹H NMR (400 MHz, CHLOROFORM-d) δ 7.49 (s, 1H), 7.42 (s, 1H), 4.59 (s, 2H), 3.68 (t, J=6.02 Hz, 2H), 2.75 (t, J=5.90 Hz, 2H), 2.26 (s, 3H), 1.48 (s, 9H), 1.35 (s, 12H).

### 3. The Preparation of tert-butyl 7-[3-ethoxy-1-(1-ethyl-4-methyl-benzotriazol-5-yl)-3-oxo-propyl]-5-methyl-3,4-dihydro-1H-isoquinoline-2-carboxylate

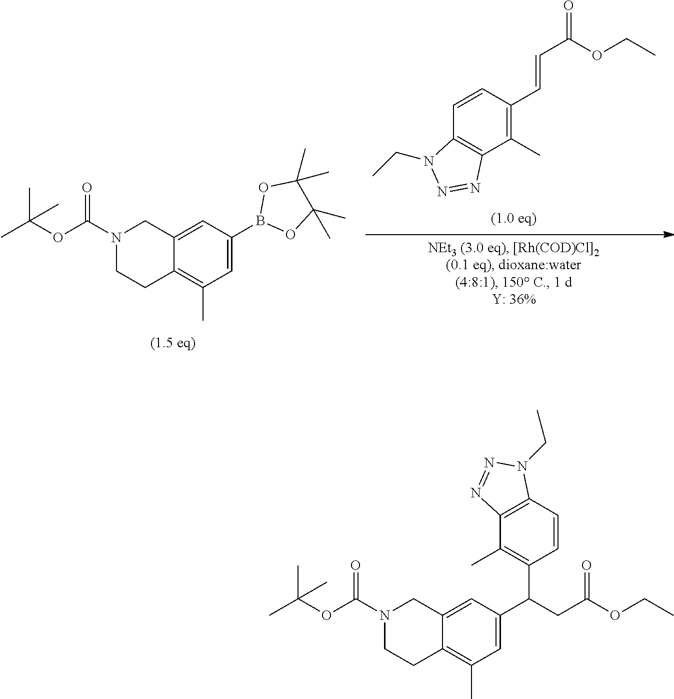

A mixture of ethyl (E)-3-(1-ethyl-4-methyl-benzotriazol-5-yl)prop-2-enoate (52.9 mg, 204 umol) and tert-butyl 5-methyl-7-(4,4,5,5-tetramethyl-1,3,2-dioxaborolan-2-yl)-3,4-dihydro-1H-isoquinoline-2-carboxylate (158 mg, 72% by mass purity, 114.1 mg, 306 umol, 1.5 eq) and triethylamine (85 uL, 612 umol, 3 eq) in dioxane (600 uL) and water (125 uL) was degassed for 10 min before chlororhodium;(1Z,5Z)-cycloocta-1,5-diene (10.0 mg, 20.4 umol, 0.1 eq) was added, and then the mixture was degassed for another 5 min. The mixture was stirred at 150° C. for 24 h in a sealed tube. The cooled reaction was diluted with water, extracted with ethyl acetate, washed with saturated sodium chloride, dried over magnesium sulfate, filtered, evaporated. The residue was purified by silica gel column (0-100% ethyl acetate in heptanes as eluent) to give the product tert-butyl 7-[3-ethoxy-1-(1-ethyl-4-methyl-benzotriazol-5-yl)-3-oxo-propyl]-5-methyl-3,4-dihydro-1H-isoquinoline-2-carboxylate (36.8 mg, 35.6% yield). ESI-MS(M+H)⁺: 507.2. ¹H NMR (400 MHz, CHLOROFORM-d) δ 7.34-7.38 (m, 1H), 7.29-7.33 (m, 1H), 6.89 (s, 1H), 6.79 (s, 1H), 4.94 (t, J=7.91 Hz, 1H), 4.65 (q, J=7.28 Hz, 2H), 4.50 (s, 2H), 4.03 (q, J=7.11 Hz, 2H), 3.63 (t, J=5.52 Hz, 2H), 3.08-3.1 (m, 1H), 2.97-3.08 (m, 1H), 2.87 (s, 3H), 2.65 (t, J=5.40 Hz, 2H), 2.18 (s, 3H), 1.59-1.64 (m, 3H), 1.43-1.53 (m, 9H), 1.11 (t, J=7.03 Hz, 3H).

### 4. The Preparation of tert-butyl 7-[3-ethoxy-1-(1-ethyl-4-methyl-benzotriazol-5-yl)-3-oxo-propyl]-5-methyl-3,4-dihydro-1H-isoquinoline-2-carboxylate enantiomer 1 and tert-butyl 7-[3-ethoxy-1-(1-ethyl-4-methyl-benzotriazol-5-yl)-3-oxo-propyl]-5-methyl-3,4-dihydro-1H-isoquinoline-2-carboxylate enantiomer 2

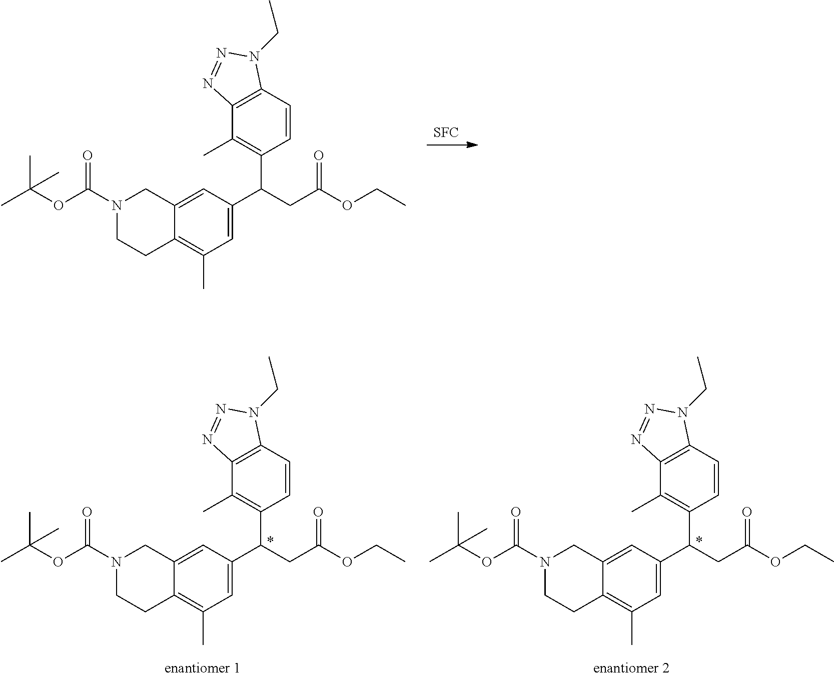

Tert-butyl 7-[3-ethoxy-1-(1-ethyl-4-methyl-benzotriazol-5-yl)-3-oxo-propyl]-5-methyl-3,4-dihydro-1H-isoquinoline-2-carboxylate (395.8 mg, 781 umol) was separated by chiral SFC chromatography (Column: CHIRALPAK AD-H 30×250 mm, 5 um; Co-solvent: 30% 2-Propanol in 0.1% DEA in CO2 (flow rate: 100 mL/min), ABPR 120 bar, MBPR 60 psi) to give the two enantiomers. Peak 1: tert-butyl 7-[3-ethoxy-1-(1-ethyl-4-methyl-benzotriazol-5-yl)-3-oxo-propyl]-5-methyl-3,4-dihydro-1H-isoquinoline-2-carboxylate enantiomer 1 (94.0 mg dried, 23.8% yield, 100% ee). ESI-MS(M+H)⁺: 507.3. Peak 2: tert-butyl 7-[3-ethoxy-1-(1-ethyl-4-methyl-benzotriazol-5-yl)-3-oxo-propyl]-5-methyl-3,4-dihydro-1H-isoquinoline-2-carboxylate enantiomer 2 (94.6 mg dried, 23.9% yield, 100% ee). ESI-MS(M+H)⁺: 507.3.

### 5. The Preparation of ethyl 3-(1-ethyl-4-methyl-benzotriazol-5-yl)-3-(5-methyl-1,2,3,4-tetrahydroisoquinolin-7-yl)propanoate enantiomer 1 trifluoroacetate salt

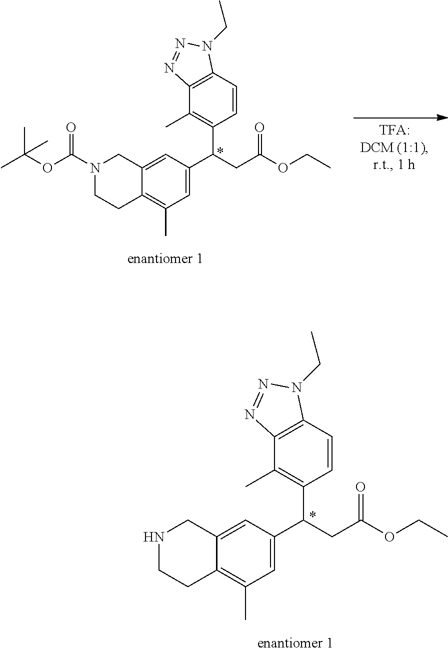 [SMILES: CCOC(=O)CC(c1cc(C)c2c(c1)CN(C(=O)OC(C)(C)C)CC2)c1ccc2c(nnn2CC)c1C ; "TFA:DCM (1:1),r.t., 1 h" ; CCOC(=O)CC(c1cc(C)c2c(c1)CNCC2)c1ccc2c(nnn2CC)c1C]

Tert-butyl 7-[(1S)-3-ethoxy-1-(1-ethyl-4-methyl-benzotriazol-5-yl)-3-oxo-propyl]-5-methyl-3,4-dihydro-1H-isoquinoline-2-carboxylate enantiomer 1 (94.0 mg, 185.5 umol) was dissolved in DCM (1.00 mL) and then trifluoroacetic acid; (1.00 mL) was added slowly. The reaction was stirred at room temperature for 1 h. The reaction was evaporated and azeotroped with DCM to give ethyl 3-(1-ethyl-4-methyl-benzotriazol-5-yl)-3-(5-methyl-1,2,3,4-tetrahydroisoquinolin-7-yl)propanoate enantiomer 1 trifluoroacetate salt. ESI-MS(M+H)⁺: 407.2.

### 6. The Preparation of ethyl 3-(1-ethyl-4-methyl-benzotriazol-5-yl)-3-[5-methyl-2-(2,3,5,6-tetramethylbenzoyl)-3,4-dihydro-1H-isoquinolin-7-yl]propanoate enantiomer 1

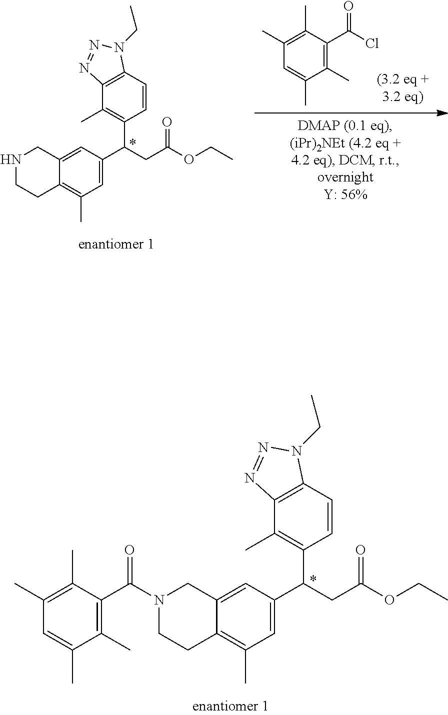 [SMILES: CCOC(=O)CC(c1cc(C)c2c(c1)CNCC2)c1ccc2c(nnn2CC)c1C ; Cc1cc(C)c(C)c(C(=O)Cl)c1C ; "DMAP (0.1 eq),(iPr)2NEt (4.2 eq +4.2 eq), DCM, r.t.,overnightY: 56%" ; CCOC(=O)CC(c1cc(C)c2c(c1)CN(C(=O)c1c(C)c(C)cc(C)c1C)CC2)c1ccc2c(nnn2CC)c1C]

3-(1-Ethyl-4-methyl-benzotriazol-5-yl)-3-(5-methyl-1,2,3,4-tetrahydroisoquinolin-7-yl)propanoate enantiomer 1 trifluoroacetate salt (92.8 umol), 2,3,5,6-tetramethylbenzoyl chloride (57.6 mg, 293 umol, 3.2 eq), DMAP (1.19 mg, 9.77 umol, 0.1 eq), diisopropylethylamine (68 uL, 391 umol, 4.2 eq) were dissolved in DCM (1.00 mL) and were stirred at RT overnight. Additional 2,3,5,6-tetramethylbenzoyl chloride (57.6 mg, 293 umol, 3.2 eq) and diisopropylethylamine (68 uL, 391 umol, 4.2 eq) were added. After 2 h, the reaction was evaporated to dryness. The residue was chromatographed on silica gel (0-100% ethyl acetate in heptanes as eluent) to give ethyl 3-(1-ethyl-4-methyl-benzotriazol-5-yl)-3-[5-methyl-2-(2,3,5,6-tetramethylbenzoyl)-3,4-dihydro-1H-isoquinolin-7-yl]propanoate enantiomer 1 (29.5 mg, 52.05 umol, 56.0% yield). ESI-MS(M+H)⁺: 567.3. ¹H NMR (400 MHz, CHLOROFORM-d) δ 7.32-7.41 (m, 1H), 7.29 (s, 1H), 6.95 (s, 1H), 6.54-6.93 (m, 2H), 4.82-5.00 (m, 2H), 4.57-4.74 (m, 2H), 3.93-4.25 (m, 3H), 3.41 (t, J=6.02 Hz, 1H), 2.92-3.20 (m, 2H), 2.77-2.90 (m, 4H), 2.56 (t, J=5.90 Hz, 1H), 2.13-2.25 (m, 9H), 2.09 (d, J=4.77 Hz, 4H), 2.00 (d, J=9.54 Hz, 3H), 1.61 (td, J=7.40, 9.54 Hz, 3H), 1.04-1.17 (m, 3H).

### 7. The Preparation of 3-(1-ethyl-4-methyl-benzotriazol-5-yl)-3-[5-methyl-2-(2,3,5,6-tetramethylbenzoyl)-3,4-dihydro-1H-isoquinolin-7-yl]propanoic acid; enantiomer 1 [68]

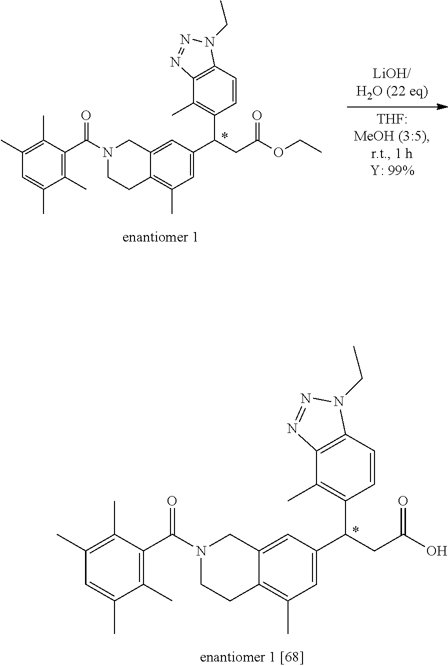

Ethyl 3-(1-ethyl-4-methyl-benzotriazol-5-yl)-3-[5-methyl-2-(2,3,5,6-tetramethylbenzoyl)-3,4-dihydro-1H-isoquinolin-7-yl]propanoate enantiomer 1 (33.9 mg, 59.8 umol) was dissolved in THF (500 uL). To this was added lithium hydroxide in water (2.6 M, 500.00 uL, 1.3 mmol, 22 eq) with stirring, followed by methanol (750 uL). After 1 h at RT, reaction was evaporated to dryness, partitioned between 1N HCl, ethyl acetate. Organics were washed with saturated sodium chloride, dried over magnesium sulfate, filtered, evaporated to give the product 3-(1-ethyl-4-methyl-benzotriazol-5-yl)-3-[5-methyl-2-(2,3,5,6-tetramethylbenzoyl)-3,4-dihydro-1H-isoquinolin-7-yl]propanoic acid; enantiomer 1 [68] (32.0 mg, 99.31% yield). ESI-MS(M+H)⁺: 539.3. ¹H NMR (400 MHz, DMSO-d6) δ 12.08 (br. s., 1H), 7.53-7.65 (m, 1H), 7.38-7.53 (m, 1H), 6.71-7.12 (m, 3H), 4.59-4.83 (m, 5H), 3.83-4.21 (m, 2H), 3.27-3.29 (m, 1H), 2.91-3.14 (m, 2H), 2.65-2.83 (m, 4H), 2.11-2.19 (m, 6H), 2.10 (s, 3H), 1.97 (s, 3H), 1.79-1.92 (m, 3H), 1.46 (q, J=7.03 Hz, 3H).

### Example 69

### 3-(1-ethyl-4-methyl-benzotriazol-5-yl)-3-[5-methyl-2-(2,3,5,6-tetramethylbenzoyl)-3,4-dihydro-1H-isoquinolin-7-yl]propanoic acid; enantiomer 2 [69]

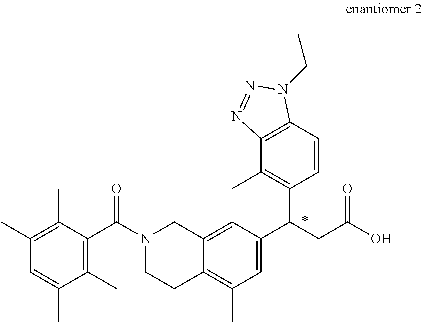 [SMILES: CCn1nnc2c(C)c(C(CC(=O)O)c3cc(C)c4c(c3)CN(C(=O)c3c(C)c(C)cc(C)c3C)CC4)ccc21]

3-(1-Ethyl-4-methyl-benzotriazol-5-yl)-3-[5-methyl-2-(2,3,5,6-tetramethylbenzoyl)-3,4-dihydro-1H-isoquinolin-7-yl]propanoic acid; enantiomer 2 [69] was synthesized as per Example 68 (Scheme VI), but using the second enantiomer of the ester tert-butyl 7-[3-ethoxy-1-(1-ethyl-4-methyl-benzotriazol-5-yl)-3-oxo-propyl]-5-methyl-3,4-dihydro-1H-isoquinoline-2-carboxylate enantiomer 2.

3-(1-Ethyl-4-methyl-benzotriazol-5-yl)-3-[5-methyl-2-(2,3,5,6-tetramethylbenzoyl)-3,4-dihydro-1H-isoquinolin-7-yl]propanoic acid; enantiomer 2 [69] (34.2 mg, 93.7%). ESI-MS(M+H)⁺: 539.2/539.3 (two peaks). ¹H NMR (400 MHz, DMSO-d6) δ 12.09 (br. s., 1H), 7.53-7.65 (m, 1H), 7.37-7.53 (m, 1H), 6.71-7.12 (m, 3H), 4.59-4.84 (m, 5H), 3.82-4.20 (m, 2H), 3.26-3.30 (m, 1H), 2.90-3.15 (m, 2H), 2.63-2.83 (m, 4H), 2.11-2.20 (m, 6H), 2.10 (s, 3H), 1.97 (s, 3H), 1.79-1.92 (m, 3H), 1.46 (q, J=7.11 Hz, 3H).

### Example 70

### (S)-3-(2-(2-naphthoyl)-1,2,3,4-tetrahydroisoquinolin-7-yl)-3-(1-ethyl-4-methyl-1H-benzo[d][1,2,3]triazol-5-yl)propanoic acid; and (R)-3-(2-(2-naphthoyl)-1,2,3,4-tetrahydroisoquinolin-7-yl)-3-(1-ethyl-4-methyl-1H-benzo[d][1,2,3]triazol-5-yl)propanoic acid;

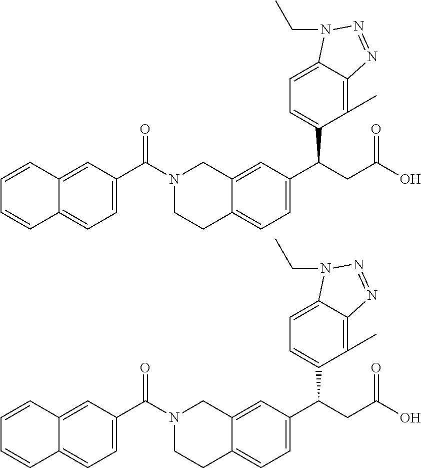

### 1. Preparation of methyl 3-(1-ethyl-4-methyl-benzotriazol-5-yl)-3-[2-(naphthalene-2-carbonyl)-3,4-dihydro-1H-isoquinolin-7-yl]propanoate

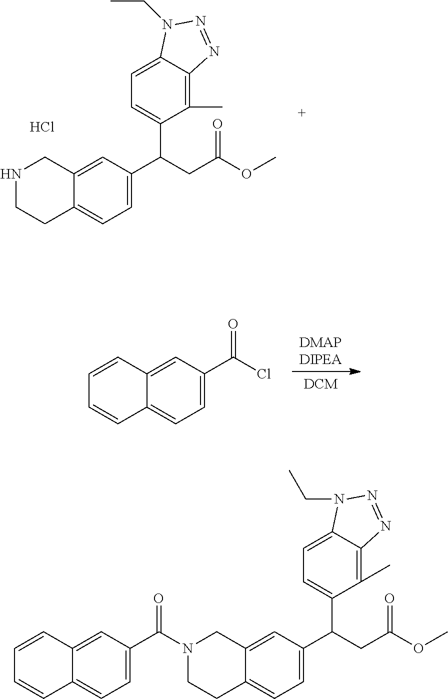

A mixture of methyl 3-(1-ethyl-4-methyl-benzotriazol-5-yl)-3-(1,2,3,4-tetrahydroisoquinolin-7-yl)propanoate (141.00 mg, 339.82 umol, Hydrochloride), naphthalene-2-carbonyl chloride (71.26 mg, 373.80 umol), and DMAP (4.15 mg, 33.98 umol) was dissolved in dichloromethane (2.00 mL) and treated with DIPEA (131.76 mg, 1.02 mmol, 178.05 uL). The reaction was stirred at rt overnight. The concentrated residue was chromatographed on silica gel (HE/EA 20-100%) to give methyl 3-(1-ethyl-4-methyl-benzotriazol-5-yl)-3-[2-(naphthalene-2-carbonyl)-3,4-dihydro-1H-isoquinolin-7-yl]propanoate (156.00 mg, 292.89 umol, 78.62% yield). LCMS: RT=1.73 min, M+H=533.

### 2. Preparation of (S)-3-(2-(2-naphthoyl)-1,2,3,4-tetrahydroisoquinolin-7-yl)-3-(1-ethyl-4-methyl-1H-benzo[d][1,2,3]triazol-5-yl)propanoic acid; and (R)-3-(2-(2-naphthoyl)-1,2,3,4-tetrahydroisoquinolin-7-yl)-3-(1-ethyl-4-methyl-1H-benzo[d][1,2,3]triazol-5-yl)propanoic acid;

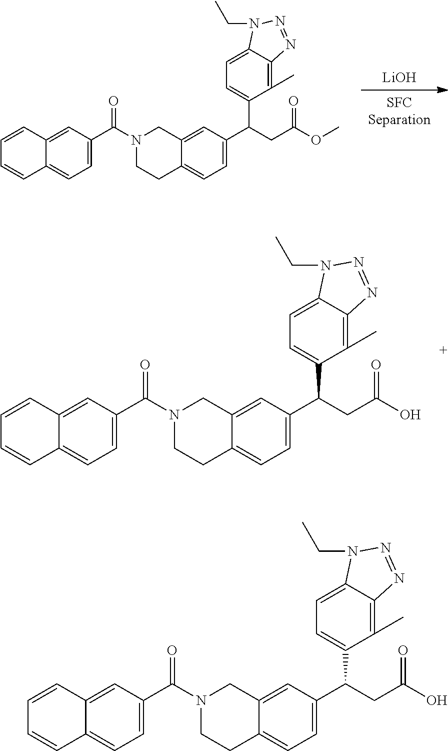 [SMILES: CCn1nnc2c(C)c(C(CC(=O)OC)c3ccc4c(c3)CN(C(=O)c3ccc5ccccc5c3)CC4)ccc21 ; "LiOH" ; "SFCSeparation" ; CCn1nnc2c(C)c([C@@H](CC(=O)O)c3ccc4c(c3)CN(C(=O)c3ccc5ccccc5c3)CC4)ccc21 ; CCn1nnc2c(C)c([C@H](CC(=O)O)c3ccc4c(c3)CN(C(=O)c3ccc5ccccc5c3)CC4)ccc21]

Methyl 3-(1-ethyl-4-methyl-benzotriazol-5-yl)-3-[2-(naphthalene-2-carbonyl)-3,4-dihydro-1H-isoquinolin-7-yl]propanoate (142.00 mg, 273.81 umol) was dissolved in 6 mL THF, 2 mL methanol and treated with aqueous lithium hydroxide (2.6 M, 2.11 mL). The reaction was allowed to stir at RT for 5 h, after which point, 1 N HCl solution was added to adjust the pH to ˜3. The aqueous phase was extracted with ethyl acetate and the combined organic extracts were dried over MgSO₄, filtered, and concentrated. Purification by chiral SFC (CHIRALPAK AS-H 30×250 mm, 5 um; Co-solvent: 35% Methanol in 0.1% DEA in CO₂ (flow rate: 100 mL/min), ABPR 120 bar, MBPR 40 psi) afforded 45.4 mg of the first eluting enantiomer as Ent-1 (ee=100%), ESI-MS (M+H)⁺: 519.1, and 45.3 mg of the second eluting enantiomer as Ent-2 (ee=97.3%). ESI-MS (M+H)⁺: 519.1. The absolute configuration was not determined.

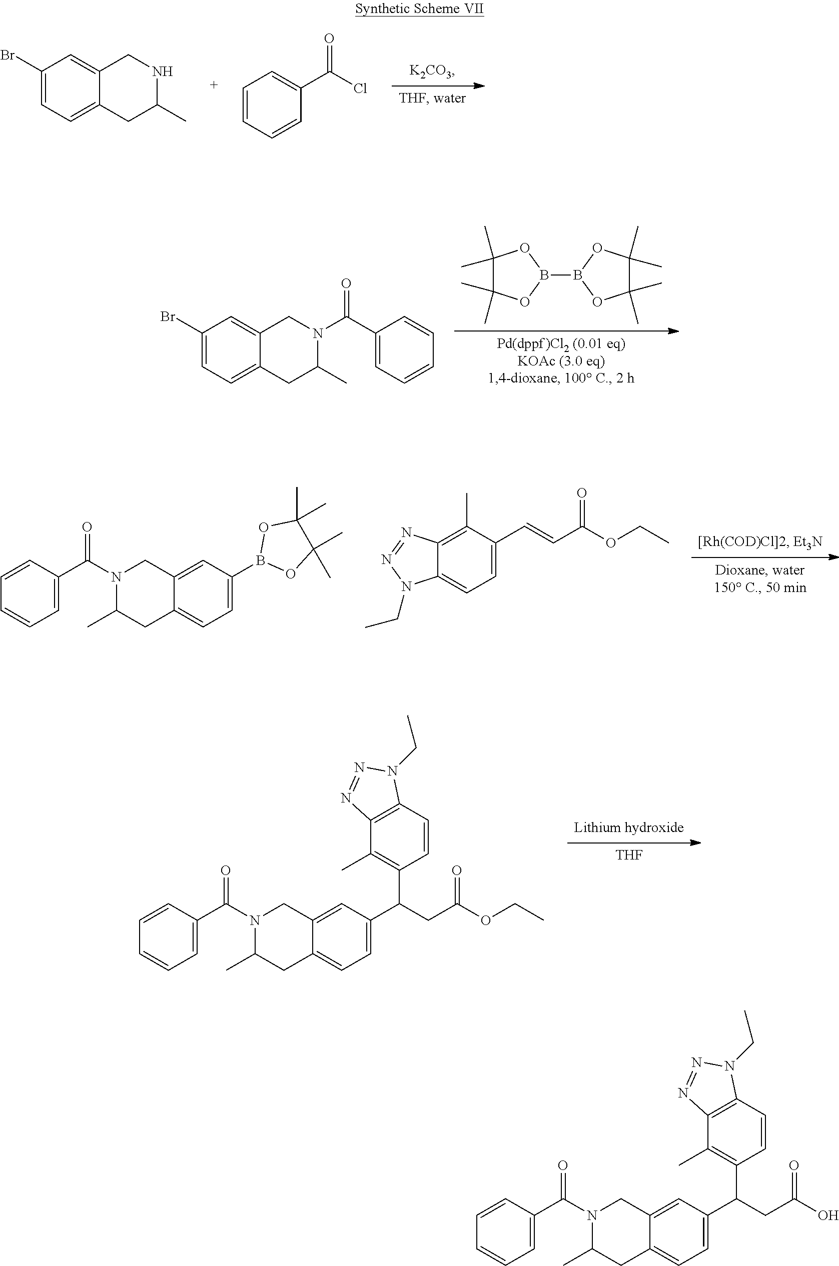

### Example 71

### 3-(2-benzoyl-3-methyl-1,2,3,4-tetrahydroisoquinolin-7-yl)-3-(1-ethyl-4-methyl-1H-benzo[d][1,2,3]triazol-5-yl)propanoic acid;

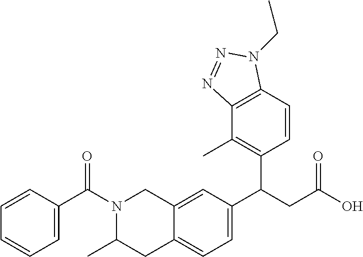 [SMILES: CCn1nnc2c(C)c(C(CC(=O)O)c3ccc4c(c3)CN(C(=O)c3ccccc3)C(C)C4)ccc21]

### 1. The Preparation of (7-bromo-3-methyl-3,4-dihydro-1H-isoquinolin-2-yl)-phenyl-methanone

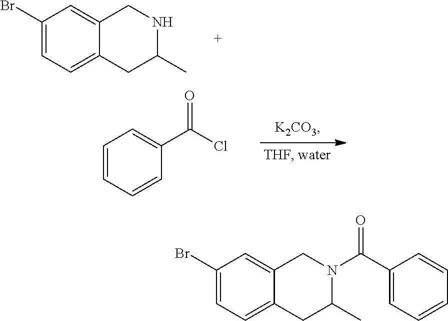

7-Bromo-3-methyl-1,2,3,4-tetrahydroisoquinoline (500.00 mg, 2.21 mmol) and Potassium carbonate (751.39 mg, 5.44 mmol) were slurried in THF (5.00 mL) and water (500.00 uL). Benzoyl chloride (310.65 mg, 2.21 mmol, 256.74 uL) was added and the reaction stirred at room temperature overnight. T reaction was diluted with EtOAc and washed with water, brine, and dried over MgSO₄. The concentrated crude product was purified with Si gel chromatography (HE/EA 0-50%) to give the desired product (621.60 mg, 1.81 mmol, 99.76% yield). ESI-MS (M+H)⁺: 301.0. ¹H NMR (400 MHz, METHANOL-d₄) δ 7.45-7.54 (m, 3H), 7.42 (br d, J=3.51 Hz, 3H), 7.34 (dd, J=2.01, 8.28 Hz, 1H), 7.09 (br d, J=7.78 Hz, 1H), 4.93-5.43 (m, 1H), 4.21-4.64 (m, 2H), 3.10 (br dd, J=4.89, 15.94 Hz, 1H), 2.51-2.78 (m, 1H), 1.01-1.41 (m, 3H)

### 2. The Preparation of [3-methyl-7-(4,4,5,5-tetramethyl-1,3,2-dioxaborolan-2-yl)-3,4-dihydro-1H-isoquinolin-2-yl]-phenyl-methanone

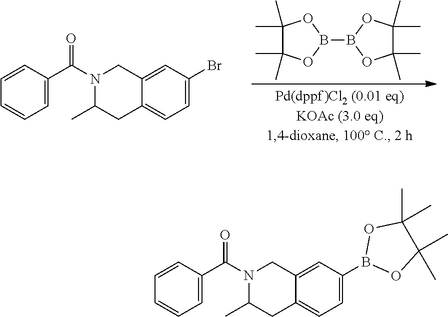

(7-Bromo-3-methyl-3,4-dihydro-1H-isoquinolin-2-yl)-phenyl-methanone (1.50 g, 4.54 mmol) and 4,4,5,5-tetramethyl-2-(4,4,5,5-tetramethyl-1,3,2-dioxaborolan-2-yl)-1,3,2-dioxaborolane (1.38 g, 5.45 mmol), dichloropalladium;triphenylphosphane (318.66 mg, 454.00 umol) and Potassium acetate (1.34 g, 13.63 mmol) were dissolved in Dioxane (16.33 g, 185.29 mmol, 15.85 mL). After degassing, the reaction was sealed and microwaved at 150° C. for 60 min. LCMS shows complete conversion to a peak consistent with the product. The reaction was diluted with ethyl acetate, washed with water, washed with saturated sodium chloride and dried over magnesium sulfate. After evaporation, the residue was chromatographed on silica gel (0-100% ethyl acetate in heptanes) to give [3-methyl-7-(4,4,5,5-tetramethyl-1,3,2-dioxaborolan-2-yl)-3,4-dihydro-1H-isoquinolin-2-yl]-phenyl-methanone. ESI-MS (M+H)⁺: 378.2.

### 3. The Preparation of ethyl 3-(2-benzoyl-3-methyl-3,4-dihydro-1H-isoquinolin-7-yl)-3-(1-ethyl-4-methyl-benzotriazol-5-yl)propanoate

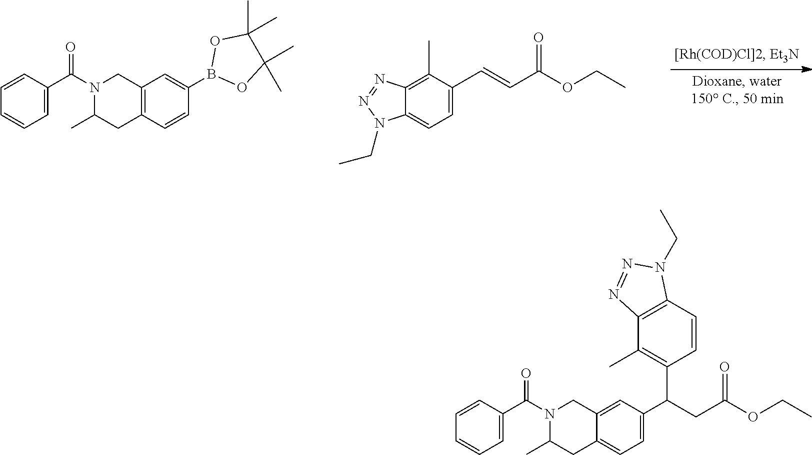 [SMILES: CC1Cc2ccc(B3OC(C)(C)C(C)(C)O3)cc2CN1C(=O)c1ccccc1 ; CCOC(=O)/C=C/c1ccc2c(nnn2CC)c1C ; CCOC(=O)CC(c1ccc2c(c1)CN(C(=O)c1ccccc1)C(C)C2)c1ccc2c(nnn2CC)c1C]

[3-Methyl-7-(4,4,5,5-tetramethyl-1,3,2-dioxaborolan-2-yl)-3,4-dihydro-1H-isoquinolin-2-yl]-phenyl-methanone (300.00 mg, 795.17 umol), ethyl (E)-3-(1-ethyl-4-methyl-benzotriazol-5-yl)prop-2-enoate (232.99 mg, 898.54 umol), N,N-diethylethanamine (241.39 mg, 2.39 mmol, 330.67 uL) and [Rh(COD)Cl]₂ (50.97 mg, 103.37 umol) were added to a solution of Dioxane (3.00 mL) and water (1.00 mL). After degassing with nitrogen, the mixture was microwaved at 150° C. for 50 min. The crude reaction was diluted with EtOAc, washed with water, saturated sodium chloride, dried over magnesium sulfate, filtered and evaporated. The concentrated residue was chromatographed on silica gel (0-100% ethyl acetate in heptanes) to give ethyl 3-(2-benzoyl-3-methyl-3,4-dihydro-1H-isoquinolin-7-yl)-3-(1-ethyl-4-methyl-benzotriazol-5-yl)propanoate. ESI-MS (M+H)⁺: 511.2.

### 4. The Preparation of 3-(2-benzoyl-3-methyl-1,2,3,4-tetrahydroisoquinolin-7-yl)-3-(1-ethyl-4-methyl-1H-benzo[d][1,2,3]triazol-5-yl)propanoic acid;

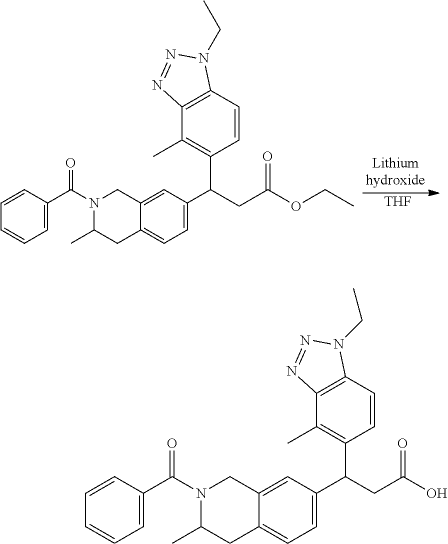 [SMILES: CCOC(=O)CC(c1ccc2c(c1)CN(C(=O)c1ccccc1)C(C)C2)c1ccc2c(nnn2CC)c1C ; CCn1nnc2c(C)c(C(CC(=O)O)c3ccc4c(c3)CN(C(=O)c3ccccc3)C(C)C4)ccc21 ; "THF"]

ethyl 3-(2-benzoyl-3-methyl-3,4-dihydro-1H-isoquinolin-7-yl)-3-(1-ethyl-4-methyl-benzotriazol-5-yl)propanoate (75.00 mg, 146.88 umol) was dissolved in THF (10.00 mL) and stirred. Lithium hydroxide 1M aq (734.40 uL, 734.40 umol) was added and the reaction stirred for 4 hrs. Organics and some of the aqueous were removed in vacuo and the crude reaction was dissolved in EtOAc and acid;ified with 1N HCl (pH 4). Solvent was removed in vacuo and crude material purified by reverse phase HPLC to afford 3-(2-benzoyl-3-methyl-1,2,3,4-tetrahydroisoquinolin-7-yl)-3-(1-ethyl-4-methyl-1H-benzo[d][1,2,3]triazol-5-yl)propanoic acid; as a white solid. ESI-MS (M+H)⁺: 483.3. ¹H NMR (400 MHz, METHANOL-d₄) δ 7.30-7.63 (m, 7H), 6.75-7.25 (m, 3H), 4.94-5.31 (m, 2H), 4.67 (q, J=7.19 Hz, 2H), 4.12-4.50 (m, 2H), 2.96-3.25 (m, 3H), 2.60-2.89 (m, 3H), 1.45-1.71 (m, 3H), 1.07-1.35 (m, 3H).

### Example 72

### 3-(2-benzoyl-3-methyl-1,2,3,4-tetrahydroisoquinolin-7-yl)-3-(1-ethyl-4-methyl-1H-benzo[d][1,2,3]triazol-5-yl)propanoic acid; Isomer 3

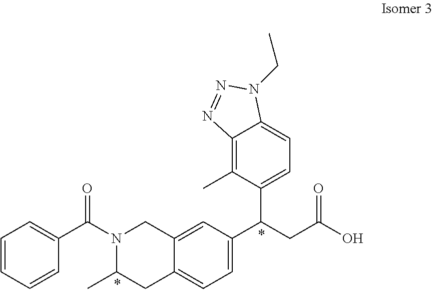 [SMILES: CCn1nnc2c(C)c(C(CC(=O)O)c3ccc4c(c3)CN(C(=O)c3ccccc3)C(C)C4)ccc21]

3-(2-Benzoyl-3-methyl-3,4-dihydro-1H-isoquinolin-7-yl)-3-(1-ethyl-4-methyl-benzotriazol-5-yl)propanoic acid; (110.00 mg, 227.95 umol) was separated into its four diastereomers by chiral SFC column. Column: CHIRALPAK AS-H 30×250 mm, 5 um. Co-solvent: 35% 2-propanol w/0.1% DEA in CO2 (flow rate: 100 mL/min), ABPR 120 bar, MBPR 60 psi. The third peak off the column was assigned as 3-(2-benzoyl-3-methyl-1,2,3,4-tetrahydroisoquinolin-7-yl)-3-(1-ethyl-4-methyl-1H-benzo[d][1,2,3]triazol-5-yl)propanoic acid; Isomer 3. ESI-MS (M+H)⁺: 483.3. ¹H NMR (400 MHz, METHANOL-d₄) δ 7.30-7.63 (m, 7H), 6.75-7.25 (m, 3H), 4.94-5.31 (m, 2H), 4.67 (q, J=7.19 Hz, 2H), 4.12-4.50 (m, 2H), 2.96-3.25 (m, 3H), 2.60-2.89 (m, 3H), 1.45-1.71 (m, 3H), 1.07-1.35 (m, 3H).

### Examples 73-82

### General Preparation of carbamide derivatives of 3-(1-ethyl-4-methyl-1H-benzo[d][1,2,3]triazol-5-yl)-3-(1,2,3,4-tetrahydroisoquinolin-7-yl)propanoic acid; through Parallel Synthesis

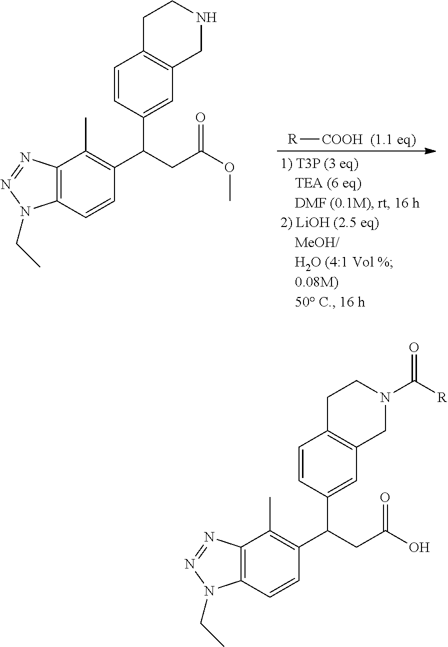

1.0 mL aliquots of a 0.1 M stock solution of the hydrochloride salt of methyl 3-(1-ethyl-4-methyl-1H-benzo[d][1,2,3]triazol-5-yl)-3-(1,2,3,4-tetrahydroisoquinolin-7-yl)propanoate (41.5 mg, 0.1 mmol) in DMF were added to the carboxylic acids (0.11 mmol), followed by the addition of triethylamine (0.6 mmol) and a 50 Vol % solution of T₃P in EtOAc (0.3 mmol). The reaction mixtures were stirred overnight at rt. Successful reactions were diluted with 2 mL EtOAc and extracted with 2 mL of a saturated NaHCO₃ solution. The aqueous layers were extracted two more times with 2 mL EtOAc. The combined organic layers were evaporated to dryness. The crude materials were then taken up in 1.0 mL methanol and 250 uL of a 1 M lithium hydroxide solution (0.25 mmol) were added. The reaction mixtures were heated to 50° C. for 16 hours.

The reaction mixtures were evaporated to dryness. The residues were taken up in 2.0 mL of a mixture of DMSO and methanol (1:1 Vol %) and were purified by prep-HPLC (MeCN/water with 0.1 Vol % ammonium hydroxide as mobile phase) to give the desired products in their free base form.

| | | | ESI-MS |
|---|---|---|---|
| Example | Product name | Mol Weight | (M + H)+ |
| 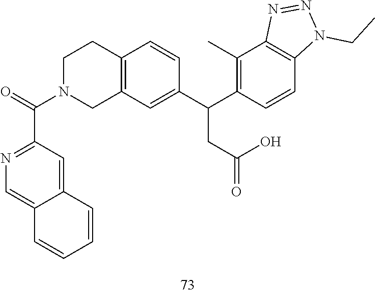 [SMILES: CCn1nnc2c(C)c(C(CC(=O)O)c3ccc4c(c3)CN(C(=O)c3cc5ccccc5cn3)CC4)ccc21] | 3-(1-ethyl-4-methyl- benzotriazol-5-y)-3-[2- (isoquinoline-3-carbonyl)- 3,4-dihydro-1H-isoquinolin- 7-yl]propanoic acid; | 519.59 | 520.2 |
| 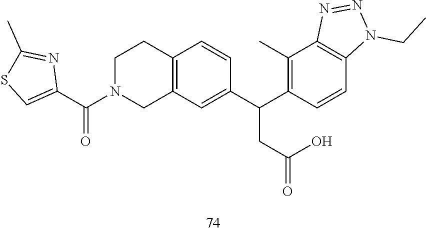 [SMILES: CCn1nnc2c(C)c(C(CC(=O)O)c3ccc4c(c3)CN(C(=O)c3csc(C)n3)CC4)ccc21] | 3-(1-ethyl-4-methyl- benzotriazol-5-y)-3-[2-(2- methylthiazole-4-carbonyl)- 3,4-dihydro-1H-isoquinolin- 7-yl]propanoic acid; | 489.59 | 490.2 |
| 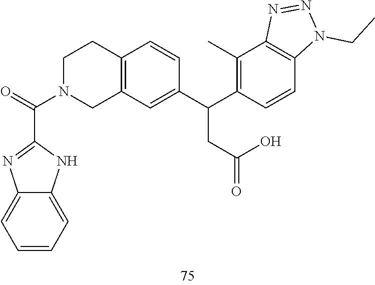 [SMILES: CCn1nnc2c(C)c(C(CC(=O)O)c3ccc4c(c3)CN(C(=O)c3nc5ccccc5[nH]3)CC4)ccc21] | 3-[2-(1H-benzimidazole-2- carbonyl)-3,4-dihydro-1H- isoquinolin-7-yl]-3-(1ethyl- 4-methyl-benzotriazol-5- yl)propanoic acid; | 508.57 | 509.3 |
| 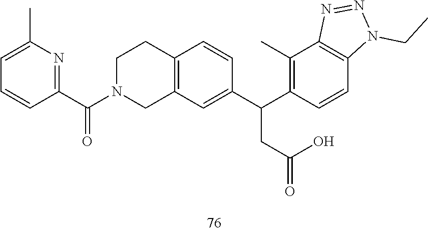 [SMILES: CCn1nnc2c(C)c(C(CC(=O)O)c3ccc4c(c3)CN(C(=O)c3cccc(C)n3)CC4)ccc21] | 3-(1-ethyl-4-methyl- benzotriazol-5-yl)-3-[2-(6- methylpyridine-2-carbonyl)- 3,4-dihydro-1H-isoquinolin- 7-yl]propanoic acid; | 483.56 | 484.2 |
| 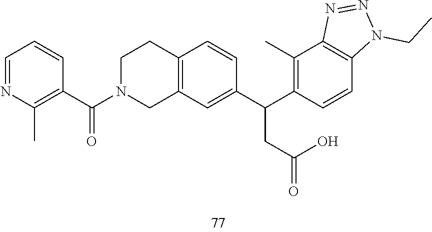 [SMILES: CCn1nnc2c(C)c(C(CC(=O)O)c3ccc4c(c3)CN(C(=O)c3cccnc3C)CC4)ccc21] | 3-(1-ethyl-4-methyl- benzotriazol-5-y)-3-[2-(2- methylpyridine-3-carbonyl)- 3,4-dihydro-1H-isoquinolin- 7-yl]propanoic acid; | 483.56 | 484.2 |
| 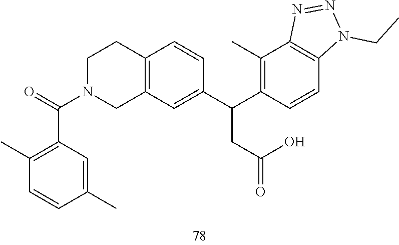 [SMILES: CCn1nnc2c(C)c(C(CC(=O)O)c3ccc4c(c3)CN(C(=O)c3cc(C)ccc3C)CC4)ccc21] | 3-[2-(2,5-dimethylbenzoyl)- 3,4-dihydro-1H- isoquinolin-7-yl]-3-(1-ethyl- 4-methyl-benzotriazol-5- yl)propanoic acid; | 496.6 | 497.3 |
| 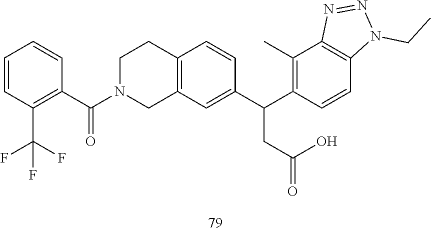 [SMILES: CCn1nnc2c(C)c(C(CC(=O)O)c3ccc4c(c3)CN(C(=O)c3ccccc3C(F)(F)F)CC4)ccc21] | 3-(1-ethyl-4-methyl- benzotriazol-5-yl)-3-[2-(2- (trifluoromethyl)benzoyl]- 3,4-dihydro-1H-isoquinolin- 7-yl]propanoic acid; | 536.54 | 537.2 |
| 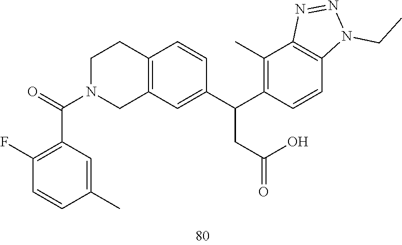 [SMILES: CCn1nnc2c(C)c(C(CC(=O)O)c3ccc4c(c3)CN(C(=O)c3cc(C)ccc3F)CC4)ccc21] | 3-(1-ethyl-4-methyl- benzotriazol-5-yl)-3-[2-(2- fluoro-5-methyl-benzoyl)- 3,4-dihydro-1H-isoquinolin- 7-yl]propanoic acid; | 500.56 | 501.2 |
| 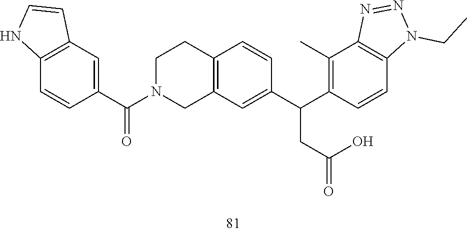 [SMILES: CCn1nnc2c(C)c(C(CC(=O)O)c3ccc4c(c3)CN(C(=O)c3ccc5[nH]ccc5c3)CC4)ccc21] | 3-(1-ethyl-4-methyl- benzotriazol-5-yl)-3-[2-(1H- indole-5-carbonyl)-3-4- dihydro-1H-isoquinolin- 7-yl]propanoic acid; | 507.58 | 508.2 |
| 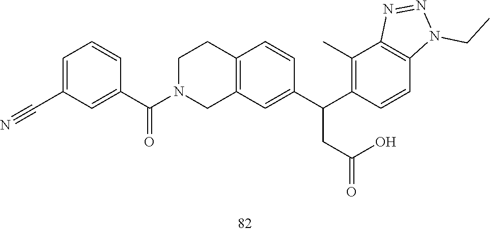 [SMILES: CCn1nnc2c(C)c(C(CC(=O)O)c3ccc4c(c3)CN(C(=O)c3cccc(C#N)c3)CC4)ccc21] | 3-[2-(3-cyanobenzoyl)-3,4- dihydro-1H-isoquinolin- 7-yl]-3-(1-ethyl-4-methyl- benzotriazol-5-yl) propanoic acid; | 493.56 | 494.2 |

### Examples 83-91

### Synthesized Following the General Procedure of Example 73

| | | | ESI-MS |
|---|---|---|---|
| Example | Product name | Mol Weight | (M + H)+ |
| 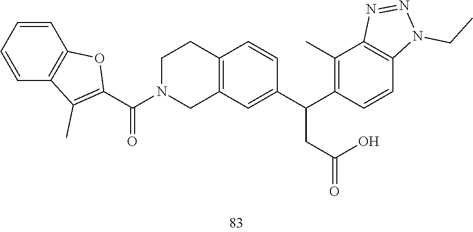 [SMILES: CCn1nnc2c(C)c(C(CC(=O)O)c3ccc4c(c3)CN(C(=O)c3oc5ccccc5c3C)CC4)ccc21] | 3-(1-ethyl-4-methyl-1H- benzo[d][1,2,3]triazol-5-yl)-3- (2-(3-methylbenzofuran-2- carbonyl)-1,2,3,4- tetrahydroisoquinolin-7- yl)propanoic acid; | 522.23 | 523.2 |
| 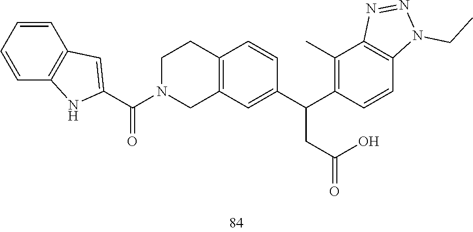 [SMILES: CCn1nnc2c(C)c(C(CC(=O)O)c3ccc4c(c3)CN(C(=O)c3cc5ccccc5[nH]3)CC4)ccc21] | 3-(2-(1H-indole-2-carbonyl)- 1,2,3,4-tetrahydroisoquinolin-7- yl)-3-(1-ethyl-4-methyl-1H- benzo[d][1,2,3]triazol-5- yl)propanoic acid; | 507.23 | 508.2 |
| 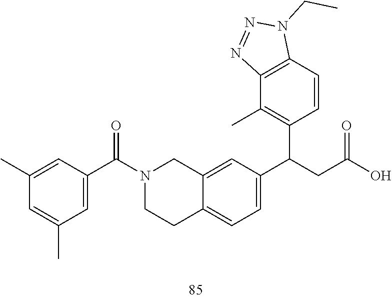 [SMILES: CCn1nnc2c(C)c(C(CC(=O)O)c3ccc4c(c3)CN(C(=O)c3cc(C)cc(C)c3)CC4)ccc21] | 3-(2-(3,5-dimethylbenzoyl)- 1,2,3,4-tetrahydroisoquinolin-7- yl)-3-(1-ethyl-4-methyl-1H- benzo[d][1,2,3]triazol-5- yl)propanoic acid; | 496.25 | 497.3 |
| 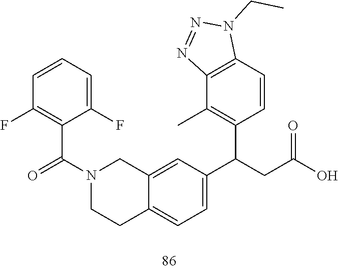 [SMILES: CCn1nnc2c(C)c(C(CC(=O)O)c3ccc4c(c3)CN(C(=O)c3c(F)cccc3F)CC4)ccc21] | 3-(2-(2,6-difluorobenzoyl)- 1,2,3,4-tetrahydroisoquinolin-7- yl)-3-(1-ethyl-4-methyl-1H- benzo[d][1,2,3]triazol-5- yl)propanoic acid; | 504.20 | 505.2 |
| 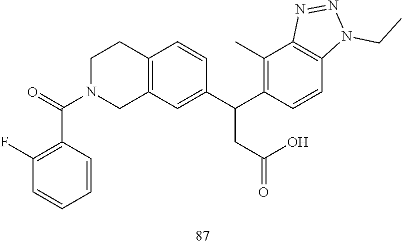 [SMILES: CCn1nnc2c(C)c(C(CC(=O)O)c3ccc4c(c3)CN(C(=O)c3ccccc3F)CC4)ccc21] | 3-(1-ethyl-4-methyl-1H- benzo[d][1,2,3]triazol-5-yl)-3- (2-(2-fluorobenzoyl)-1,2,3,4- tetrahydroisoquinolin-7- yl)propanoic acid; | 486.21 | 487.1 |
| 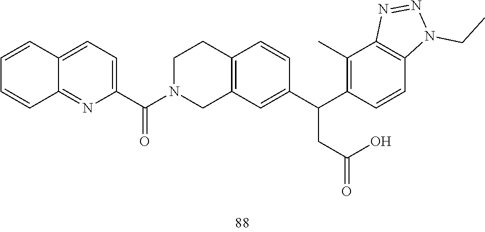 [SMILES: CCn1nnc2c(C)c(C(CC(=O)O)c3ccc4c(c3)CN(C(=O)c3ccc5ccccc5n3)CC4)ccc21] | 3-(1-ethyl-4-methyl-1H- benzo[d][1,2,3]triazol-5-yl)-3- (2-(quinoline-2- carbonyl)-1,2,3,4- tetrahydroisoquinolin-7- yl)propanoic acid; | 519.23 | 520.2 |
| 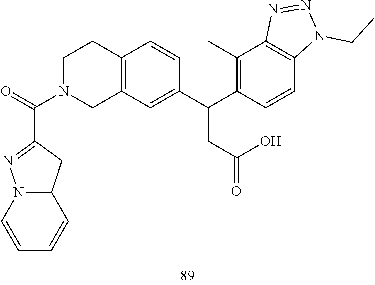 [SMILES: CCn1nnc2c(C)c(C(CC(=O)O)c3ccc4c(c3)CN(C(=O)C3=NN5C=CC=CC5C3)CC4)ccc21] | 3-(1-ethyl-4-methyl-1H- benzo[d][1,2,3]triazol-5-yl)-3- (2-(pyrazolo[1,5-a]pyridine-2- carbonyl)-1,2,3,4- tetrahydroisoquinolin-7- yl)propanoic acid; | 508.22 | 509.2 |
| 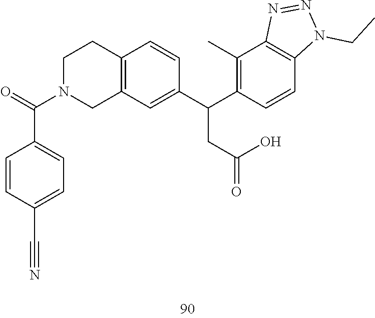 [SMILES: CCn1nnc2c(C)c(C(CC(=O)O)c3ccc4c(c3)CN(C(=O)c3ccc(C#N)cc3)CC4)ccc21] | 3-(2-(4-cyanobenzoyl)-1,2,3,4- tetrahydroisoquinolin-7-yl)-3- (1-ethyl-4-methyl-1H- benzo[d][1,2,3]triazol-5- yl)propanoic acid | 493.21 | 494.1 |
| 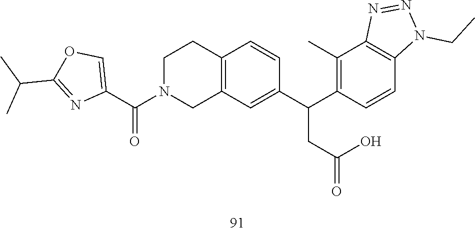 [SMILES: CCn1nnc2c(C)c(C(CC(=O)O)c3ccc4c(c3)CN(C(=O)c3coc(C(C)C)n3)CC4)ccc21] | 3-(1-ethyl-4-methyl-1H- benzo[d][1,2,3]triazol-5-yl)-3- (2-(2-isopropyloxazole-4- carbonyl)-1,2,3,4- tetrahydroisoquinolin-7- yl)propanoic acid; | 501.24 | 502.2 |

### Examples 92 and 93

### (S)-3-(2-(2,5-dimethylbenzoyl)-1,2,3,4-tetrahydroisoquinolin-7-yl)-3-(6-methoxy-4-methylpyridin-3-yl)propanoic acid and (R)-3-(2-(2,5-dimethylbenzoyl)-1,2,3,4-tetrahydroisoquinolin-7-yl)-3-(6-methoxy-4-methylpyridin-3-yl)propanoic acid

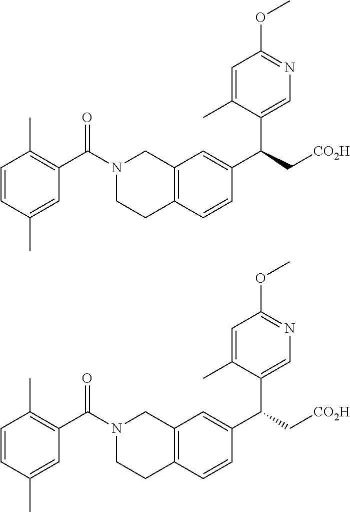 [SMILES: COc1cc(C)c([C@@H](CC(=O)O)c2ccc3c(c2)CN(C(=O)c2cc(C)ccc2C)CC3)cn1 ; COc1cc(C)c([C@H](CC(=O)O)c2ccc3c(c2)CN(C(=O)c2cc(C)ccc2C)CC3)cn1]

### 1. Preparation of tert-butyl 7-(4,4,5,5-tetramethyl-1,3,2-dioxaborolan-2-yl)-3,4-dihydroisoquinoline-2(1H)-carboxylate

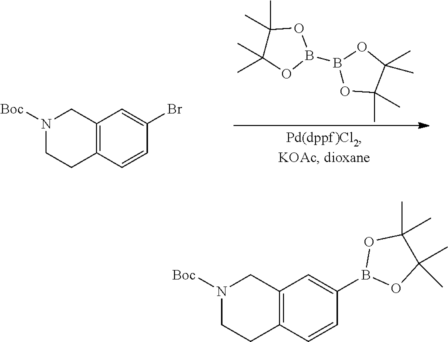

To a solution of compound tert-butyl 7-bromo-3,4-dihydroisoquinoline-2(1H)-carboxylate (4.0 g, 0.0128 mol, 1.0 eq.) and compound PinBBPin (4.87 g, 0.0192 mol, 1.5 eq.) in dioxane (100 mL) was added KOAc (2.5 g, 0.0256 mol, 2.0 eq.) and Pd(dppf)Cl₂ (0.94 g, 0.00128 mol, 0.1 eq.). The mixture solution was stirred at 100° C. for 5 h under N₂ atmosphere. LCMS showed the starting material was almost consumed and a new spot was observed. The mixture was concentrated to give the residue, which was diluted with H₂O (20 mL) and extracted with DCM (25 mL×3). The combined organic layer was dried over Na₂SO₄ and filtered. The filtrate was concentrated to give the residue, which was purified by column chromatography on silica gel (PE:EA=100:1 to 8:1) to give the compound tert-butyl 7-(4,4,5,5-tetramethyl-1,3,2-dioxaborolan-2-yl)-3,4-dihydroisoquinoline-2(1H)-carboxylate (4.0 g, yield: 87%) as a solid. ¹HNMR: (400 MHz, CDCl₃) δ=7.51-7.67 (m, 2H), 7.15 (d, J=7.4 Hz, 1H), 4.58 (s, 2H), 3.64 (s, 2H), 2.85 (s, 2H), 1.48 (s, 10H), 1.30-1.38 (m, 12H).

### 2. Preparation of ethyl (E)-3-(6-methoxy-4-methylpyridin-3-yl)acrylate

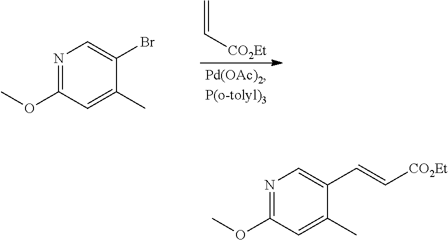

To a solution of compound 5-bromo-2-methoxy-4-methylpyridine (3.5 g, 0.017 mol, 1.0 eq.) and compound ethyl acrylate (8.5 g, 0.085 mol, 5.0 eq.) in DMF (40 mL) was added DIEA (6.6 g, 0.051 mol, 3.0 eq.) and P(o-tolyl)₃ (2.06 g, 0.0068 mol, 0.4 eq.) and Pd(OAc)₂ (760 mg, 0.0034 mol, 0.2 eq.). The mixture solution was stirred at 110° C. for 18 h under N₂ atmosphere. LCMS showed the starting material was almost consumed and the desired product was observed. The mixture was concentrated to give the residue, which was diluted with H₂O (15 mL) and extracted with EA (20 mL×3). The combined organic layer was dried over Na₂SO₄ and filtered. The filtrate was concentrated to give the residue, which was purified by column chromatography on silica gel (PE:EA=100:1 to 20:1) to give the compound ethyl (E)-3-(6-methoxy-4-methylpyridin-3-yl)acrylate (3.0 g, yield: 80%) as a yellow solid. ¹HNMR: (400 MHz, CDCl₃) δ=8.33 (s, 1H), 7.83-7.88 (d, J=20.0 Hz, 2H), 6.85 (s, 1H), 6.30-6.34 (d, J=16.0 Hz, 2H), 4.24-4.30 (m, 2H), 3.94 (s, 3H), 2.38 (s, 3H),1.33-1.36 (m, 3H).

### 3. Preparation of tert-butyl 7-(3-ethoxy-1-(6-methoxy-4-methylpyridin-3-yl)-3-oxopropyl)-3,4-dihydroisoquinoline-2(1H)-carboxylate

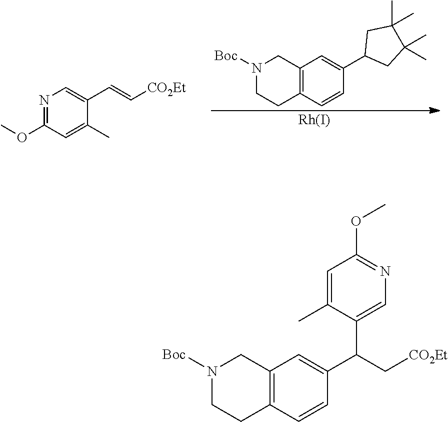

To a solution of ethyl (E)-3-(6-methoxy-4-methylpyridin-3-yl)acrylate (200 mg, 0.9 mmol, 2.0 eq.) and tert-butyl 7-(4,4,5,5-tetramethyl-1,3,2-dioxaborolan-2-yl)-3,4-dihydroisoquinoline-2(1H)-carboxylate (161 mg, 0.45 mmol, 1.0 eq.) in dioxane (3 mL) and H₂O (1 mL) was added TEA (136 mg, 1.35 mmol, 3.0 eq.) and [RhCl(cod)]₂ (11 mg, 0.0225 mmol, 0.05 eq.). The mixture was stirred at 110° C. for 18 h under N₂ atmosphere. LCMS showed the starting material was almost consumed and the desired product was observed. The mixture was diluted with H₂O (5 mL) and extracted with EA (8 mL×3). The combined organic layer was dried over Na₂SO₄ and filtered. The filtrate was concentrated to give the residue, which was purified by prep-TLC (PE:EA=8:1) to give the compound tert-butyl 7-(3-ethoxy-1-(6-methoxy-4-methylpyridin-3-yl)-3-oxopropyl)-3,4-dihydroisoquinoline-2(1H)-carboxylate (100 mg, yield: 25%) as an oil. MS: (M+H: 545.2). ¹HNMR: (400 MHz, CDCl₃) δ=8.05 (s, 1H), 6.94-7.08 (m, 2H), 6.88 (s, 1H), 6.52 (s, 1H), 4.57 (t, J=8.3 Hz, 1H), 4.48 (s, 2H), 4.06 (m, 2H), 3.90 (s, 3H), 3.60 (s, 2H), 3.00 (m, 2H), 2.76 (s, 2H), 2.20 (s, 3H), 1.57 (s, 10H), 1.15 (t, J=7.0 Hz, 3H).

### 4. Preparation of ethyl 3-(6-methoxy-4-methylpyridin-3-yl)-3-(1,2,3,4-tetrahydroisoquinolin-7-yl)propanoate

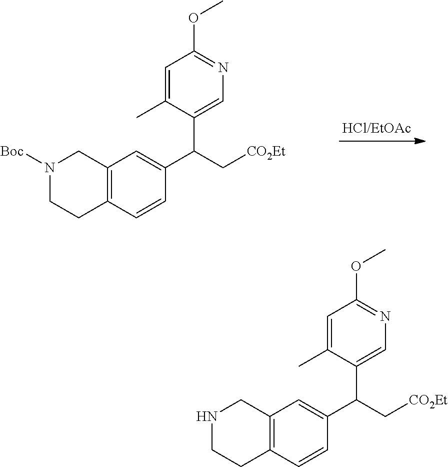 [SMILES: CCOC(=O)CC(c1ccc2c(c1)CN(C(=O)OC(C)(C)C)CC2)c1cnc(OC)cc1C ; CCOC(=O)CC(c1ccc2c(c1)CNCC2)c1cnc(OC)cc1C]

To a solution of compound tert-butyl 7-(3-ethoxy-1-(6-methoxy-4-methylpyridin-3-yl)-3-oxopropyl)-3,4-dihydroisoquinoline-2(1H)-carboxylate (450 mg, 0.99 mmol, 1.0 eq.) in EA (2.5 mL) was added HCl/EA (2.5 mL). The mixture was stirred at 10° C.-15° C. for 3 h. TLC (PE:EA=2:1) showed the starting material was almost consumed and the desired product was observed. The mixture was concentrated to give the compound ethyl 3-(6-methoxy-4-methylpyridin-3-yl)-3-(1,2,3,4-tetrahydroisoquinolin-7-yl)propanoate (350 mg, yield: 100%) as a solid. MS: (M+H: 355.1).

### 5. Preparation of ethyl (S)-3-(2-(2,5-dimethylbenzoyl)-1,2,3,4-tetrahydroisoquinolin-7-yl)-3-(6-methoxy-4-methylpyridin-3-yl)propanoate and ethyl (R)-3-(2-(2,5-dimethylbenzoyl)-1,2,3,4-tetrahydroisoquinolin-7-yl)-3-(6-methoxy-4-methylpyridin-3-yl)propanoate

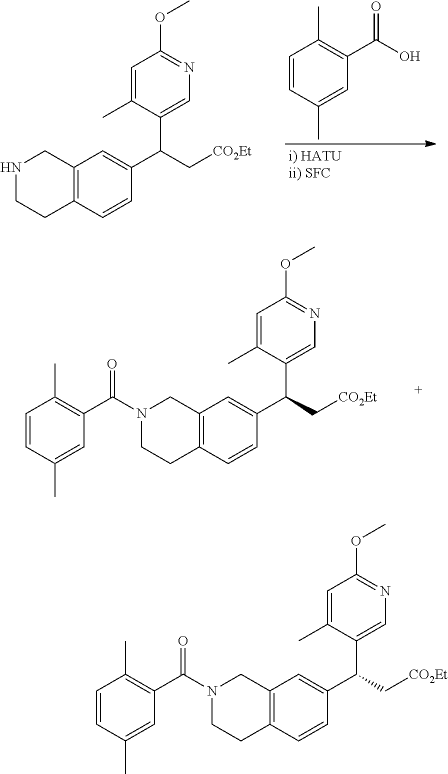 [SMILES: CCOC(=O)CC(c1ccc2c(c1)CNCC2)c1cnc(OC)cc1C ; Cc1ccc(C)c(C(=O)O)c1 ; CCOC(=O)C[C@@H](c1ccc2c(c1)CN(C(=O)c1cc(C)ccc1C)CC2)c1cnc(OC)cc1C ; CCOC(=O)C[C@H](c1ccc2c(c1)CN(C(=O)c1cc(C)ccc1C)CC2)c1cnc(OC)cc1C]

To a solution of compound ethyl 3-(6-methoxy-4-methylpyridin-3-yl)-3-(1,2,3,4-tetrahydroisoquinolin-7-yl)propanoate (170 mg, 0.565 mmol, 1.0 eq.) and 2,5-dimethylbenzoic acid (127 mg, 0.85 mmol, 1.5 eq.) in DCM (5 mL) was added HATU (323 mg, 0.85 mmol, 1.5 eq.) and TEA (228 mg, 2.26 mmol, 4.0 eq.). The mixture was stirred at 10° C.-15° C. for 3 h under N₂ atmosphere. TLC (PE:EA=2:1) showed the starting material was almost consumed and the desired product was observed. The mixture was diluted with H₂O (10 mL) and extracted with EA (15 mL×3). The combined organic layer was dried over Na₂SO₄ and filtered. The filtrate was concentrated to give the residue, which was purified by prep-TLC (PE:EA=2:1) to give ethyl 3-(2-(2,5-dimethylbenzoyl)-1,2,3,4-tetrahydroisoquinolin-7-yl)-3-(6-methoxy-4-methylpyridin-3-yl)propanoate (200 mg, yield: 72.7%) as a solid. The racemic ester (200 mg) was separated by SFC (Column: AS (250 mm*30 mm, 5 um); Mobile phase: Neu-MeOH; Flow Rate: 55 mL/min) to supply ethyl (S)-3-(2-(2,5-dimethylbenzoyl)-1,2,3,4-tetrahydroisoquinolin-7-yl)-3-(6-methoxy-4-methylpyridin-3-yl)propanoate (50 mg, yield: 25%) and ethyl (R)-3-(2-(2,5-dimethylbenzoyl)-1,2,3,4-tetrahydroisoquinolin-7-yl)-3-(6-methoxy-4-methylpyridin-3-yl)propanoate (50 mg, yield: 25%) as a white solid. MS: (M+H: 487.2).

### 6. Preparation of (S)-3-(2-(2,5-dimethylbenzoyl)-1,2,3,4-tetrahydroisoquinolin-7-yl)-3-(6-methoxy-4-methylpyridin-3-yl)propanoic acid

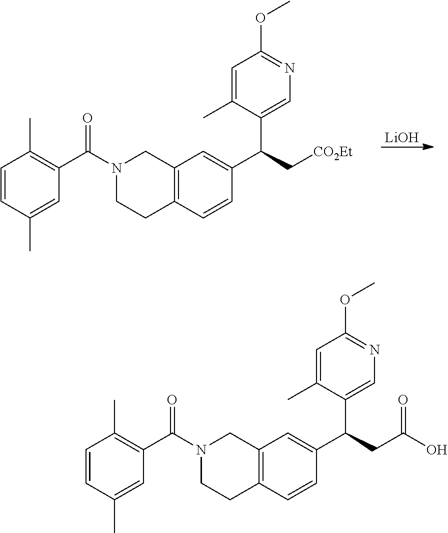 [SMILES: CCOC(=O)C[C@@H](c1ccc2c(c1)CN(C(=O)c1cc(C)ccc1C)CC2)c1cnc(OC)cc1C ; "LiOH" ; COc1cc(C)c([C@@H](CC(=O)O)c2ccc3c(c2)CN(C(=O)c2cc(C)ccc2C)CC3)cn1]

To a solution of compound ethyl (S)-3-(2-(2,5-dimethylbenzoyl)-1,2,3,4-tetrahydroisoquinolin-7-yl)-3-(6-methoxy-4-methylpyridin-3-yl)propanoate (50 mg, 0.1 mmol, 1.0 eq.) in EtOH (3 mL) and H₂O (1 mL) was added LiOH (21 mg, 0.5 mmol, 5.0 eq.). The reaction mixture was stirred at 10° C.-15° C. for 15 h. LCMS showed the starting material was almost consumed and the desired product was observed. The mixture was diluted with water (10 mL) and acidified with 1 N HCl until pH=4 and extracted with EtOAc (15 mL×3). The combined organic layer was dried over Na₂SO₄ and filtered. The filtrate was concentrated to give (S)-3-(2-(2,5-dimethylbenzoyl)-1,2,3,4-tetrahydroisoquinolin-7-yl)-3-(6-methoxy-4-methylpyridin-3-yl)propanoic acid (Rt=3.933 min, 10 mg, 18% yield, purity: 98.68%, ee value=98.24%) as a white solid. MS: (M+H: 459.2). ¹HNMR: (400 MHz, MeOD) δ=7.87-8.25 (m, 1H), 6.51-7.34 (m, 7H), 4.75-5.06 (m, 1H), 4.52-4.70 (m, 1H), 4.26-4.44 (m, 1H), 3.87 (d, J=11.8 Hz, 4H), 3.48 (d, J=3.8 Hz, 1H), 2.73-3.12 (m, 4H), 2.28-2.36 (m, 3H), 2.24 (d, J=3.9 Hz, 3H), 2.03-2.21 (m, 3H).

### 7. Preparation of (R)-3-(2-(2,5-dimethylbenzoyl)-1,2,3,4-tetrahydroisoquinolin-7-yl)-3-(6-methoxy-4-methylpyridin-3-yl)propanoic acid

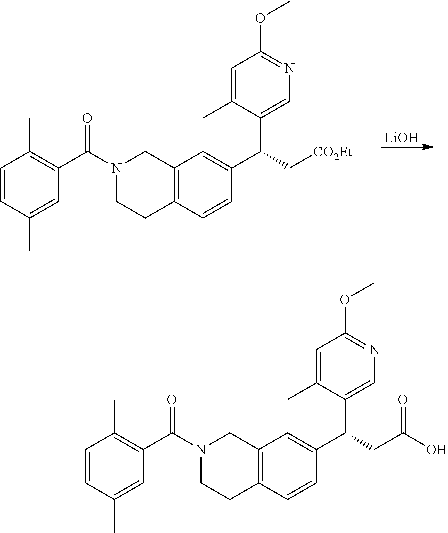 [SMILES: CCOC(=O)C[C@H](c1ccc2c(c1)CN(C(=O)c1cc(C)ccc1C)CC2)c1cnc(OC)cc1C ; "LiOH" ; COc1cc(C)c([C@H](CC(=O)O)c2ccc3c(c2)CN(C(=O)c2cc(C)ccc2C)CC3)cn1]

To a solution of compound ethyl (R)-3-(2-(2,5-dimethylbenzoyl)-1,2,3,4-tetrahydroisoquinolin-7-yl)-3-(6-methoxy-4-methylpyridin-3-yl)propanoate (50 mg, 0.1 mmol, 1.0 eq.) in EtOH (3 mL) and H₂O (1 mL) was added LiOH (21 mg, 0.5 mmol, 5.0 eq.). The reaction mixture was stirred at 10° C.-15° C. for 15 h. LCMS showed the starting material was almost consumed and the desired product was observed. The mixture was diluted with water (10 mL) and acidified with 1 N HCl until pH=4 and extracted with EtOAc (15 mL×3). The combined organic layer was dried over Na₂SO₄ and filtered. The filtrate was concentrated to give (R)-3-(2-(2,5-dimethylbenzoyl)-1,2,3,4-tetrahydroisoquinolin-7-yl)-3-(6-methoxy-4-methylpyridin-3-yl)propanoic acid (Rt=4.658 min, 7 mg, 15% yield, purity: 100%, ee value=98.72%) as a white solid. MS: (M+H : 459.2). ¹HNMR: (400 MHz, MeOD) δ=7.91-8.21 (m, 1H), 6.52-7.28 (m, 7H), 4.76-5.05 (m, 1H), 4.52-4.69 (m, 1H), 4.25-4.44 (m, 1H), 3.87 (d, J=11.2 Hz, 4H), 3.48 (d, J=3.8 Hz, 1H), 2.71-3.15 (m, 4H), 2.29-2.36 (m, 3H), 2.25 (d, J=4.4 Hz, 3H), 2.02-2.21 (m, 3H).

### Examples 94 and 95

### (S)-3-(2-(2-fluoro-5-methylbenzoyl)-1,2,3,4-tetrahydroisoquinolin-7-yl)-3-(6-methoxy-4-methylpyridin-3-yl)propanoic acid and (R)-3-(2-(2-fluoro-5-methylbenzoyl)-1,2,3,4-tetrahydroisoquinolin-7-yl)-3-(6-methoxy-4-methylpyridin-3-yl)propanoic acid

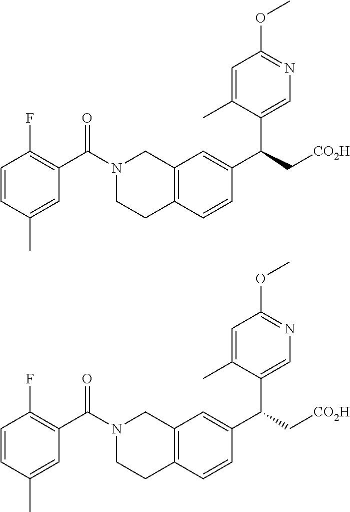 [SMILES: COc1cc(C)c([C@@H](CC(=O)O)c2ccc3c(c2)CN(C(=O)c2cc(C)ccc2F)CC3)cn1 ; COc1cc(C)c([C@H](CC(=O)O)c2ccc3c(c2)CN(C(=O)c2cc(C)ccc2F)CC3)cn1]

### 1. Preparation of Ethyl (S)-2-(2-(2-fluoro-5-methylbenzoyl)-1,2,3,4-tetrahydroisoquinolin-7-yl)-2-(6-methoxy-4-methylpyridin-3-yl)propionate and Ethyl (R)-2-(2-(2-fluoro-5-methylbenzoyl)-1,2,3,4-tetrahydroisoquinolin-7-yl)-2-(6-methoxy-4-methylpyridin-3-yl) propionate

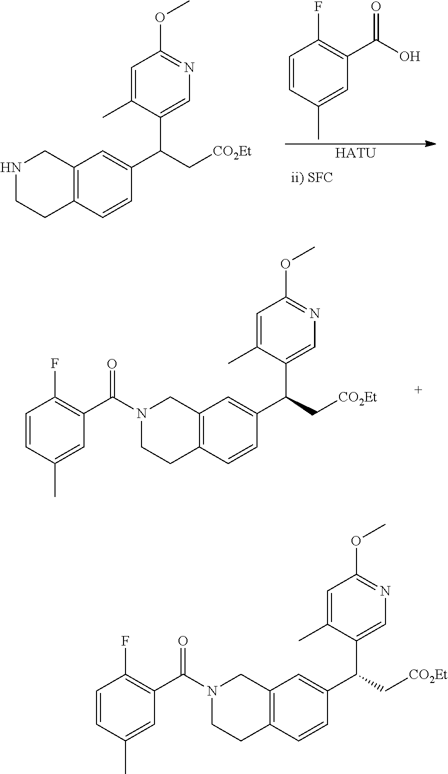

To a solution of ethyl 3-(6-methoxy-4-methylpyridin-3-yl)-3-(1,2,3,4-tetrahydroisoquinolin-7-yl)propanoate (150 mg, 0.42 mmol, 1.0 eq.) and 2-fluoro-5-methylbenzoic acid (97 mg, 0.63 mmol, 1.5 eq.) in DCM (5 mL) was added HATU (239.4 mg, 0.63 mmol, 1.5 eq.) and TEA (170 mg, 1.68 mmol, 4.0 eq.). The mixture was stirred at 10° C.-15° C. for 3 h. TLC (PE:EA=2:1) showed the starting material was almost consumed and the desired product was observed. The mixture was diluted with H₂O (10 mL) and extracted with DCM (10 mL×3). The combined organic layer was dried over Na₂SO₄ and filtered. The filtrate was concentrated to give the residue, which was purified by prep-TLC (PE:EA=2:1) to give the Ethyl 2-(2-(2-fluoro-5-methylbenzoyl)-1,2,3,4-tetrahydroisoquinolin-7-yl)-2-(6-methoxy-4-methylpyridin-3-yl) propionate (150 mg, yield: 73%) as a solid. The compound Ethyl 2-(2-(2-fluoro-5-methylbenzoyl)-1,2,3,4-tetrahydroisoquinolin-7-yl)-2-(6-methoxy-4-methylpyridin-3-yl) propionate (150 mg) was separated by SFC (Column: AS (250 mm*30 mm, 5 um); Mobile phase: 0.1% NH₃H₂O ETOH; Flow Rate: 55 mL/min) to supply Ethyl (S)-2-(2-(2-fluoro-5-methylbenzoyl)-1,2,3,4-tetrahydroisoquinolin-7-yl)-2-(6-methoxy-4-methylpyridin-3-yl) propionate (50 mg, yield: 33%) and Ethyl (R)-2-(2-(2-fluoro-5-methylbenzoyl)-1,2,3,4-tetrahydroisoquinolin-7-yl)-2-(6-methoxy-4-methylpyridin-3-yl) propionate (50 mg, yield: 33%) as a white solid. LCMS: (M+H: 491.2).

### 2. Preparation of (S)-3-(2-(2-fluoro-5-methylbenzoyl)-1,2,3,4-tetrahydroisoquinolin-7-yl)-3-(6-methoxy-4-methylpyridin-3-yl)propanoic acid

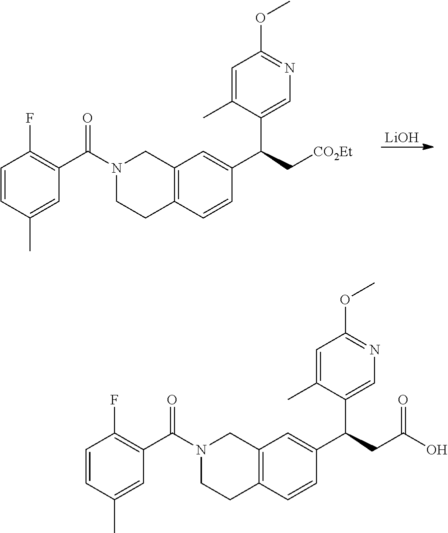 [SMILES: CCOC(=O)C[C@@H](c1ccc2c(c1)CN(C(=O)c1cc(C)ccc1F)CC2)c1cnc(OC)cc1C ; "LiOH" ; COc1cc(C)c([C@@H](CC(=O)O)c2ccc3c(c2)CN(C(=O)c2cc(C)ccc2F)CC3)cn1]

To a solution of compound Ethyl (S)-2-(2-(2-fluoro-5-methylbenzoyl)-1,2,3,4-tetrahydroisoquinolin-7-yl)-2-(6-methoxy-4-methylpyridin-3-yl) propionate (50 mg, 0.1 mmol, 1.0 eq.) in EtOH (3mL) and H₂O (1 mL) was added LiOH (21 mg, 0.5 mmol, 5.0 eq.). The reaction mixture was stirred at 10° C.-15° C. for 15 h. LCMS showed the starting material was almost consumed and the desired product was observed. The mixture was diluted with water (10 mL) and acidified with 1 N FA until pH=5 and extracted with EtOAc (15 mL×3). The combined organic layer was dried over Na₂SO₄ and filtered. The filtrate was concentrated to give (S)-3-(2-(2-fluoro-5-methylbenzoyl)-1,2,3,4-tetrahydroisoquinolin-7-yl)-3-(6-methoxy-4-methylpyridin-3-yl)propanoic acid (Rt=4.005 min, 10 mg, 18% yield, purity: 100%, ee=99.5%) as a white solid. LCMS: (M+H: 463.2). ¹HNMR: (400 MHz, MeOD) δ 7.92-8.14 (m, 1H), 6.51-7.38 (m, 7H), 4.84 (m, 1H), 4.54-4.69 (m, 1H), 4.47 (s, 1H), 3.85-4.06 (m, 4H), 3.56 (t, J=5.92 Hz, 1H), 2.77-3.13 (m, 4H), 2.35 (d, J=12.2 Hz, 3H), 2.16-2.26 (m, 3H).

### 3. Preparation of (R)-3-(2-(2-fluoro-5-methylbenzoyl)-1,2,3,4-tetrahydroisoquinolin-7-yl)-3-(6-methoxy-4-methylpyridin-3-yl)propanoic acid

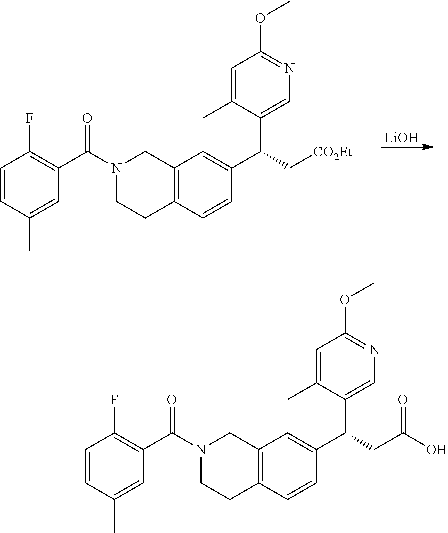 [SMILES: CCOC(=O)C[C@H](c1ccc2c(c1)CN(C(=O)c1cc(C)ccc1F)CC2)c1cnc(OC)cc1C ; "LiOH" ; COc1cc(C)c([C@H](CC(=O)O)c2ccc3c(c2)CN(C(=O)c2cc(C)ccc2F)CC3)cn1]

To a solution of Ethyl (R)-2-(2-(2-fluoro-5-methylbenzoyl)-1,2,3,4-tetrahydroisoquinolin-7-yl)-2-(6-methoxy-4-methylpyridin-3-yl) propionate (50 mg, 0.1 mmol, 1.0 eq.) in EtOH (3 mL) and H₂O (1 mL) was added LiOH (21 mg, 0.5 mmol, 5.0 eq.). The reaction mixture was stirred at 10° C.-15° C. for 15 h. LCMS showed the starting material was almost consumed and the desired product was observed. The mixture was diluted with water (10 mL) and acidified by formic acid until pH=5 and extracted with EtOAc (15 mL×3). The combined organic layer was dried over Na₂SO₄ and filtered. The filtrate was concentrated to give (R)-3-(2-(2-fluoro-5-methylbenzoyl)-1,2,3,4-tetrahydroisoquinolin-7-yl)-3-(6-methoxy-4-methylpyridin-3-yl)propanoic acid (Rt=4.622 min, 9 mg, 18% yield, purity: 100%, ee=95.98%) as a white solid. LCMS: (M+1: 463.2). ¹HNMR: (400 MHz, MeOD) δ=7.95-8.15 (m, 1H), 6.50-7.40 (m, 7H), 4.84 (m, 1H), 4.54-4.69 (m, 1H), 4.47 (s, 1H), 3.87 (m, 4H), 3.57 (t, J=5.92 Hz, 1H), 2.88-3.13 (m, 3H), 2.82 (m 1H), 2.36 (d, J=11.2 Hz, 3H), 2.27-2.13 (m, 3H).

### Example 96

### 3-[2-(3,4-dimethylbenzoyl)-3,4-dihydro-1H-isoquinolin-7-yl]-3-(6-methoxy-4-methyl-3-pyridyl)propanoic acid

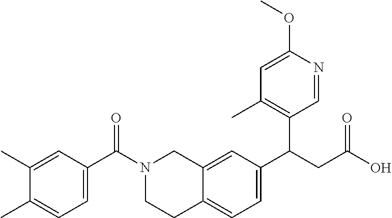 [SMILES: COc1cc(C)c(C(CC(=O)O)c2ccc3c(c2)CN(C(=O)c2ccc(C)c(C)c2)CC3)cn1]

### 1. Preparation of methyl 3-[2-(3,4-dimethylbenzoyl)-3,4-dihydro-1H-isoquinolin-7-yl]-3-(6-methoxy-4-methyl-3-pyridyl)propanoate

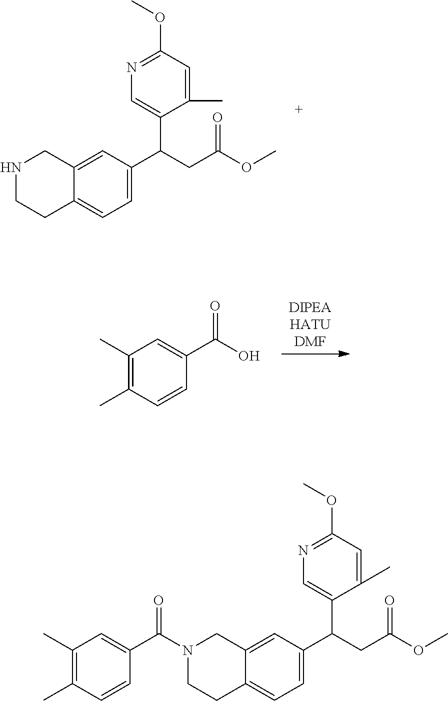

methyl 3-(6-methoxy-4-methyl-3-pyridyl)-3-(1,2,3,4-tetrahydroisoquinolin-7-yl)propanoate (24.00 mg, 63.68 umol, Hydrochloride), 3,4-dimethylbenzoic acid (14.35 mg, 95.52 umol), DIPEA (24.69 mg, 191.04 umol, 33.36 uL), HATU (36.42 mg, 95.52 umol) in DMF (1.00 mL) was stirred at rt for overnight. The crude was purified with prep HPLC to give methyl 3-[2-(3,4-dimethylbenzoyl)-3,4-dihydro-1H-isoquinolin-7-yl]-3-(6-methoxy-4-methyl-3-pyridyl)propanoate (10.00 mg, 21.16 umol, 33.23% yield). LCMS: Rt=1.40 min, m/z=473.2.

### 2. Preparation of 3-[2-(3,4-dimethylbenzoyl)-3,4-dihydro-1H-isoquinolin-7-yl]-3-(6-methoxy-4-methyl-3-pyridyl)propanoic acid

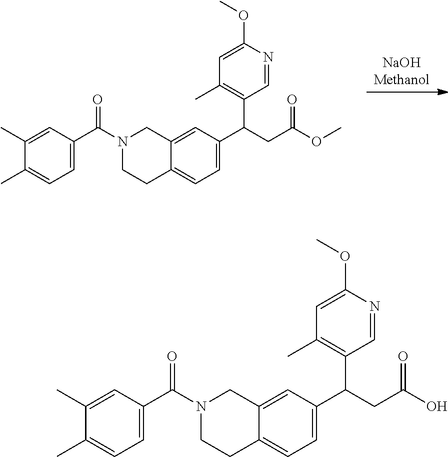

methyl 3-[2-(3,4-dimethylbenzoyl)-3,4-dihydro-1H-isoquinolin-7-yl]-3-(6-methoxy-4-methyl-3-pyridyl)propanoate (10.00 mg, 21.16 umol) in methanol (1.00 mL) was added NaOH (2 M, 21.16 uL) and microwaved at 100° C. for 30 min. After neutralization with 2M HCl, the crude was purified with prep HPLC to give 3-[2-(3,4-dimethylbenzoyl)-3,4-dihydro-1H-isoquinolin-7-yl]-3-(6-methoxy-4-methyl-3-pyridyl)-propanoic acid (3.20 mg, 6.63 umol, 31.33% yield, 95% purity). LCMS: Rt=1.23 min, m/z=459.2. ¹H NMR (400 MHz, CHLOROFORM-d) δ 8.36 (br. s., 1H), 6.54-7.25 (m, 7H), 2.62-5.24 (m, 12H), 2.13-2.48 (m, 9H).

### Example 97

### 3-[2-(3,4-dimethylbenzoyl)-3,4-dihydro-1H-isoquinolin-7-yl]-3-(6-methoxy-4-methyl-3-pyridyl)-2-methyl-propanoic acid

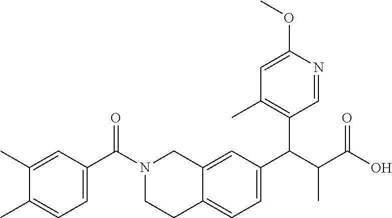 [SMILES: COc1cc(C)c(C(c2ccc3c(c2)CN(C(=O)c2ccc(C)c(C)c2)CC3)C(C)C(=O)O)cn1]

### 1. Preparation of tert-butyl 7-[3-methoxy-1-(6-methoxy-4-methyl-3-pyridyl)-2-methyl-3-oxo-propyl]-3,4-dihydro-1H-isoquinoline-2-carboxylate

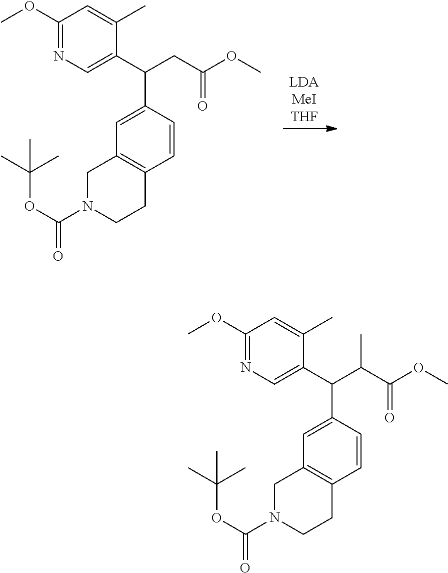

LDA (1 M, 517.56 uL) in THF (0.5 ml) was cooled to −78° C. and tert-butyl 7-[3-methoxy-1-(6-methoxy-4-methyl-3-pyridyl)-3-oxo-propyl]-3,4-dihydro-1H-isoquinoline-2-carboxylate (190.00 mg, 431.30 umol) in THF (2.00 mL) was added and stirred for 2 h. After warmed to −20° C., MeI (90.69 mg, 646.95 umol, 88.91 uL) was added and warmed to rt overnight. After quenching with water, the EtOAc extract was washed with brine and dried over Na₂SO₄, the concentrated residue was chromatographed on Si gel (HE/EA 0-60%) to give tert-butyl 7-[3-methoxy-1-(6-methoxy-4-methyl-3-pyridyl)-2-methyl-3-oxo-propyl]-3,4-dihydro-1H-isoquinoline-2-carboxylate (20.40 mg, 44.88 umol, 10.41% yield). LCMS: Rt=1.58 min, m/z=454.2.

### 2. Preparation of methyl 3-(6-methoxy-4-methyl-3-pyridyl)-2-methyl-3-(1,2,3,4-tetrahydroisoquinolin-7-yl)propanoate

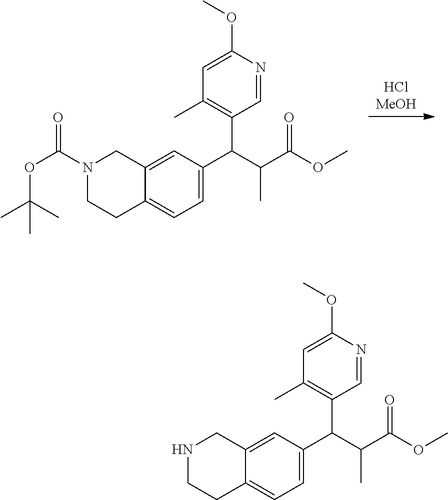 [SMILES: COC(=O)C(C)C(c1ccc2c(c1)CN(C(=O)OC(C)(C)C)CC2)c1cnc(OC)cc1C ; COC(=O)C(C)C(c1ccc2c(c1)CNCC2)c1cnc(OC)cc1C ; "HClMeOH"]

tert-butyl 7-[3-methoxy-1-(6-methoxy-4-methyl-3-pyridyl)-2-methyl-3-oxo-propyl]-3,4-dihydro-1H-isoquinoline-2-carboxylate (34.00 mg, 74.80 umol) in MeOH (2.00 mL) was added HCl (4 M, 37.40 uL) and stirred overnight. LCMS: Rt=0.74 min, m/z=355.3. The crude was used as is.

### 3. Preparation of methyl 3-[2-(3,4-dimethylbenzoyl)-3,4-dihydro-1H-isoquinolin-7-yl]-3-(6-methoxy-4-methyl-3-pyridyl)-2-methyl-propanoate

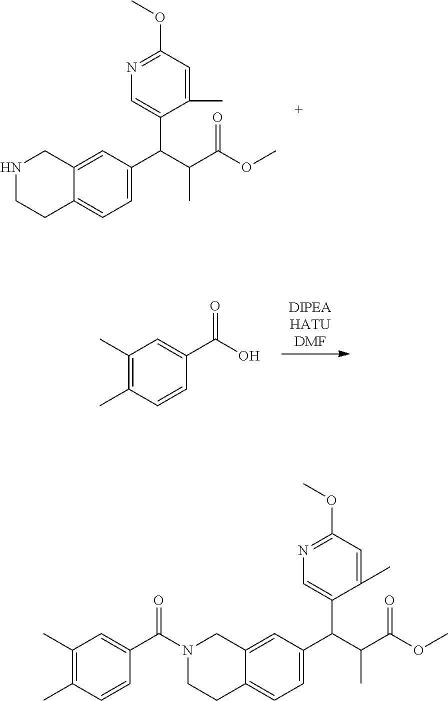

methyl 3-(6-methoxy-4-methyl-3-pyridyl)-2-methyl-3-(1,2,3,4-tetrahydroisoquinolin-7-yl)-propanoate (20.50 mg, 57.84 umol), 3,4-dimethylbenzoic acid (13.03 mg, 86.76 umol), DIPEA (22.42 mg, 173.52 umol, 30.30 uL), and HATU (33.08 mg, 86.76 umol) in DMF (1.50 mL) was stirred at rt for overnight. LCMS: Rt=1.51 min, m/z=487.2. After quenching with MeOH, the crude was purified with prep HPLC to give methyl 3-[2-(3,4-dimethylbenzoyl)-3,4-dihydro-1H-isoquinolin-7-yl]-3-(6-methoxy-4-methyl-3-pyridyl)-2-methyl-propanoate (22.80 mg, 46.86 umol, 81.01% yield).

### 4. Preparation of 3-[2-(3,4-dimethylbenzoyl)-3,4-dihydro-1H-isoquinolin-7-yl]-3-(6-methoxy-4-methyl-3-pyridyl)-2-methyl-propanoic acid

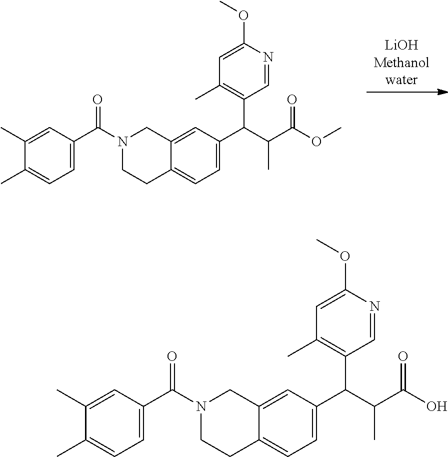

methyl 3-[2-(3,4-dimethylbenzoyl)-3,4-dihydro-1H-isoquinolin-7-yl]-3-(6-methoxy-4-methyl-3-pyridyl)-2-methyl-propanoate (22.80 mg, 46.86 umol) in Methanol (750.01 uL) was added LiOH (2.24 mg, 93.72 umol) and microwaved at 100° C. for 30 min. After neutralized with 2 M HCl, the crude was purified with prep HPLC to give 3-[2-(3,4-dimethylbenzoyl)-3,4-dihydro-1H-isoquinolin-7-yl]-3-(6-methoxy-4-methyl-3-pyridyl)-2-methyl-propanoic acid (6.60 mg, 13.27 umol, 28.31% yield, 95% purity). LCMS: Rt=1.30 min, m/z=473.2. ¹H NMR (400 MHz, CHLOROFORM-d) δ 8.13-8.73 (m, 1H), 6.47-7.24 (m, 7H), 2.66-5.10 (m, 11H), 2.44 (br. s., 3H), 2.29 (s, 6H), 0.89-1.39 (m, 3H).

### Example 98

### 3-(2-(3,4-dimethylbenzoyl)-1,2,3,4-tetrahydroisoquinolin-7-yl)-3-(4-methoxy-2-methylphenyl)propanoic acid

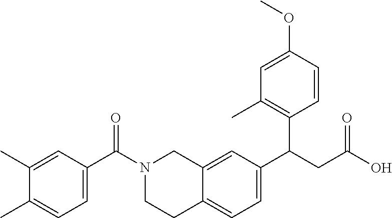 [SMILES: COc1ccc(C(CC(=O)O)c2ccc3c(c2)CN(C(=O)c2ccc(C)c(C)c2)CC3)c(C)c1]

### 1. Preparation of (7-bromo-3,4-dihydroisoquinolin-2(1H)-yl)(3,4-dimethylphenyl)methanone

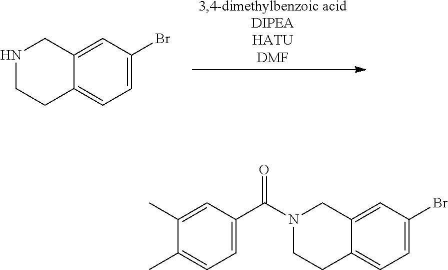 [SMILES: "3,4-dimethylbenzoic acidDIPEAHATUDMF" ; Brc1ccc2c(c1)CNCC2 ; Cc1ccc(C(=O)N2CCc3ccc(Br)cc3C2)cc1C]

7-bromo-1,2,3,4-tetrahydroisoquinoline (3.00 g, 12.07 mmol, Hydrochloride), 3,4-dimethylbenzoic acid (2.18 g, 14.48 mmol), N-ethyl-N-isopropyl-propan-2-amine (4.68 g, 36.21 mmol, 6.32 mL), HATU (5.52 g, 14.48 mmol) in DMF (20.00 mL) was stirred at rt for overnight. After dilution with EtOAc and filtration through Celite, the concentrated residue was chromatographed to give (7-bromo-3,4-dihydro-1H-isoquinolin-2-yl)-(3,4-dimethylphenyl)methanone (1.96 g, 5.69 mmol, 47.17% yield). LCMS: Rt=1.82 min, m/z=344.1.

### 2. Preparation of (3,4-dimethylphenyl)-[7-(4,4,5,5-tetramethyl-1,3,2-dioxaborolan-2-yl)-3,4-dihydro-1H-isoquinolin-2-yl]methanone

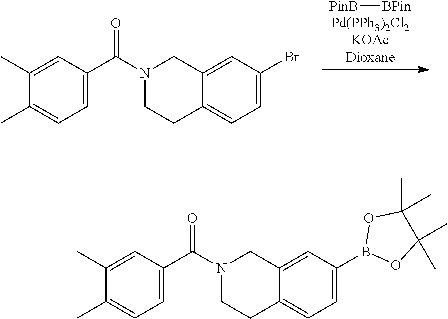

(7-bromo-3,4-dihydro-1H-isoquinolin-2-yl)-(3,4-dimethylphenyl)methanone (655.00 mg, 1.90 mmol), 4,4,5,5-tetramethyl-2-(4,4,5,5-tetramethyl-1,3,2-dioxaborolan-2-yl)-1,3,2-dioxaborolane (724.77 mg, 2.85 mmol), dichloropalladium;triphenylphosphane (133.55 mg, 190.27 umol), Potassium acetate (560.21 mg, 5.71 mmol) in Dioxane (8.00 mL) was refluxed for overnight. After dilution with EtOAc and filtration through Celite, the concentrated residue was chromatographed on Si gel (HE/EA 0-100%) to give (3,4-dimethylphenyl)-[7-(4,4,5,5-tetramethyl-1,3,2-dioxaborolan-2-yl)-3,4-dihydro-1H-isoquinolin-2-yl]methanone (736.00 mg, 1.79 mmol, 94.04% yield, 95% purity). LCMS: Rt=2.00 min, m/z=392.3.

### 3. Preparation of methyl (E)-3-(4-methoxy-2-methyl-phenyl)prop-2-enoate

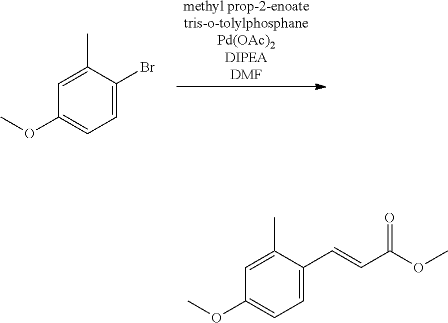

1-bromo-4-methoxy-2-methyl-benzene (500.00 mg, 2.49 mmol, 352.11 uL), methyl prop-2-enoate (2.14 g, 24.90 mmol, 2.23 mL), tris-o-tolylphosphane (151.58 mg, 498.00 umol), Pd(OAc)₂ (55.90 mg, 249.00 umol), DIPEA (965.42 mg, 7.47 mmol, 1.30 mL) in DMF (6.00 mL) was microwaved at 120° C. for 2 h. After dilution with EtOAc and filtration, the solution was washed with water and brine and dried. The concentrated residue was chromatographed on Si gel (HE/EA 0-100%) to give methyl (E)-3-(4-methoxy-2-methyl-phenyl)prop-2-enoate (311.00 mg, 1.51 mmol, 60.56% yield). ¹H NMR (400 MHz, CHLOROFORM-d) δ 7.94 (d, J=16.06 Hz, 1H), 7.54 (d, J=8.53 Hz, 1H), 6.64-6.88 (m, 2H), 6.28 (d, J=15.81 Hz, 1H), 3.82 (d, J=9.04 Hz, 6H), 2.44 (s, 3H).

### 4. Preparation of methyl 3-[2-(3,4-dimethylbenzoyl)-3,4-dihydro-1H-isoquinolin-7-yl]-3-(4-methoxy-2-methyl-phenyl)propanoate

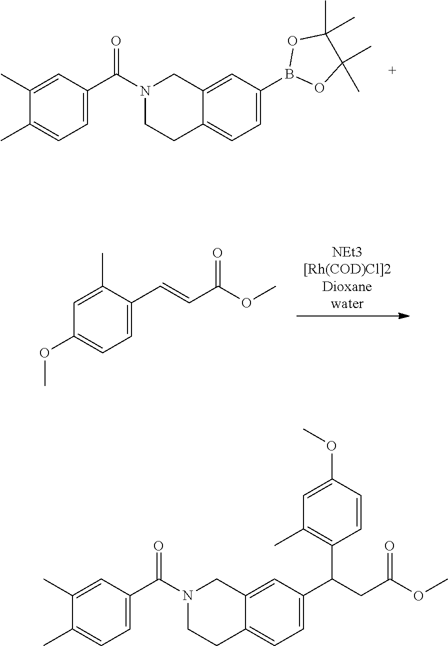

(3,4-dimethylphenyl)-[7-(4,4,5,5-tetramethyl-1,3,2-dioxaborolan-2-yl)-3,4-dihydro-1H-isoquinolin-2-yl]methanone (284.60 mg, 727.30 umol), methyl (E)-3-(4-methoxy-2-methyl-phenyl)prop-2-enoate (100.00 mg, 484.87 umol), N,N-diethylethanamine (147.19 mg, 1.45 mmol, 201.63 uL), chlororhodium;(1Z,5Z)-cycloocta-1,5-diene (13.93 mg, 48.49 umol) in Dioxane (1.50 mL) and water (500.00 uL) was microwaved at 150° C. for 50 min. The crude was diluted with EtOAc and washed with brine and dried over Na₂SO₄. The concentrated residue was chromatographed on Si gel (HE/EA 0-100%) to give methyl 3-[2-(3,4-dimethylbenzoyl)-3,4-dihydro-1H-isoquinolin-7-yl]-3-(4-methoxy-2-methyl-phenyl)propanoate (78.70 mg, 158.54 umol, 32.70% yield, 95% purity). LCMS: Rt=1.90 min, m/z=472.2.

### 5. Preparation of 3-(2-(3,4-dimethylbenzoyl)-1,2,3,4-tetrahydroisoquinolin-7-yl)-3-(4-methoxy-2-methylphenyl)propanoic acid

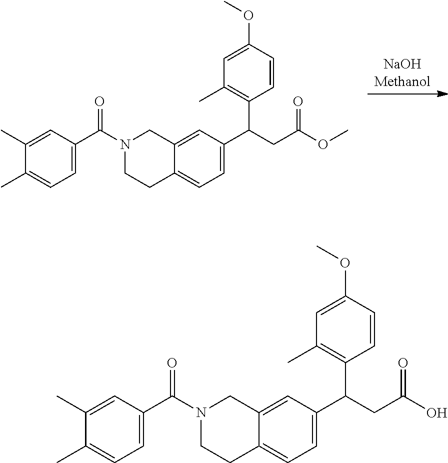

methyl 3-(2-(3,4-dimethylbenzoyl)-1,2,3,4-tetrahydroisoquinolin-7-yl)-3-(4-methoxy-2-methylphenyl)propanoate (78.70 mg, 166.88 umol) in Methanol (2.00 mL) was added NaOH (2 M, 166.88 uL) and microwaved at 100° C. for 30 min. After neutralized with 2M HCl, the crude was purified with prep HPLC to give 3-(2-(3,4-dimethylbenzoyl)-1,2,3,4-tetrahydroisoquinolin-7-yl)-3-(4-methoxy-2-methylphenyl)propanoic acid (20.00 mg, 41.52 umol, 24.88% yield, 95% purity). LCMS: Rt=1.67 min, m/z=458.2. ¹H NMR (400 MHz, CHLOROFORM-d) δ 6.41-7.26 (m, 9H), 2.69-5.19 (m, 12H), 2.30 (d, J=10.04 Hz, 9H).

### Example 99

### 3-(1,4-dimethylbenzotriazol-5-yl)-3-[2-(3,4-dimethylbenzoyl)-3,4-dihydro-1H-isoquinolin-7-yl]propanoic acid

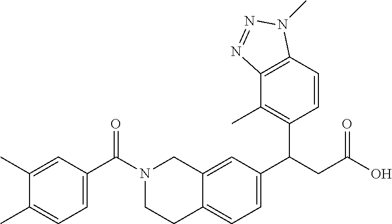 [SMILES: Cc1ccc(C(=O)N2CCc3ccc(C(CC(=O)O)c4ccc5c(nnn5C)c4C)cc3C2)cc1C]

### 1. Preparation of methyl (E)-3-(1,4-dimethylbenzotriazol-5-yl)prop-2-enoate

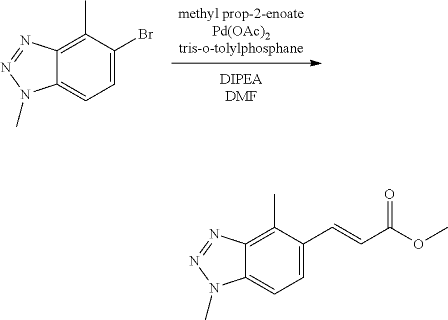 [SMILES: Cc1c(Br)ccc2c1nnn2C ; "methyl prop-2-enoatePd(OAc)2tris-o-tolylphosphane" ; COC(=O)/C=C/c1ccc2c(nnn2C)c1C]

5-bromo-1,4-dimethyl-benzotriazole (500.00 mg, 2.21 mmol), methyl prop-2-enoate (1.90 g, 22.10 mmol, 1.98 mL), Pd(OAc)₂ (49.62 mg, 221.00 umol), tris-o-tolylphosphane (134.53 mg, 442.00 umol), DIPEA (856.86 mg, 6.63 mmol, 1.16 mL) in DMF (5.00 mL) was microwaved at 120° C. for 2 h. After dilution with EtOAc and washing with water, the Na₂SO₄ dried residue was concentrated and chromatographed on Si gel (HE/EA 0-100%) to give methyl (E)-3-(1,4-dimethylbenzotriazol-5-yl)prop-2-enoate (147.20 mg, 636.54 umol, 28.80% yield). LCMS: Rt=1.16 min, m/z=232.1.

### 2. Preparation of methyl 3-(1,4-dimethylbenzotriazol-5-yl)-3-[2-(3,4-dimethylbenzoyl)-3,4-dihydro-1H-isoquinolin-7-yl]propanoate

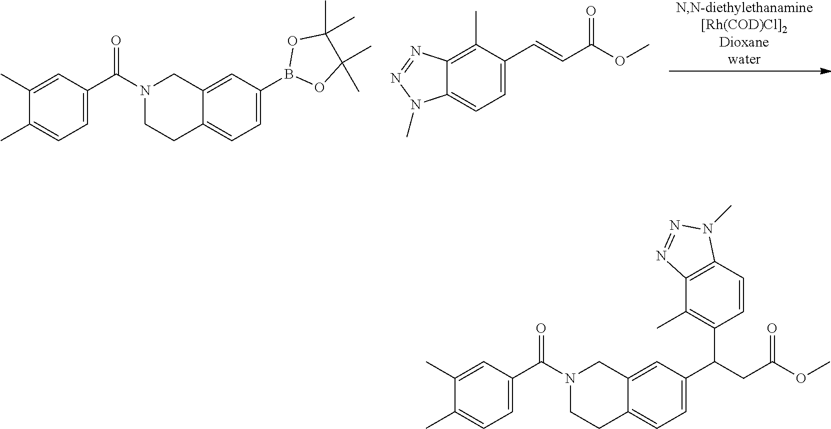

(3,4-dimethylphenyl)-[7-(4,4,5,5-tetramethyl-1,3,2-dioxaborolan-2-yl)-3,4-dihydro-1H-isoquinolin-2-yl]methanone (350.28 mg, 895.14 umol), methyl (E)-3-(1,4-dimethylbenzotriazol-5-yl)prop-2-enoate (138.00 mg, 596.76 umol), N,N-diethylethanamine (181.16 mg, 1.79 mmol, 248.16 uL), chlororhodium;(1Z,5Z)-cycloocta-1,5-diene (17.14 mg, 59.68 umol) in Dioxane (1.50 mL) and water (500.00 uL) was microwaved at 150° C. for 50 min. After dilution with EtOAc and filtration through celite, the dried concentrated residue was chromatographed on Si gel to give methyl 3-(1,4-dimethylbenzotriazol-5-yl)-3-[2-(3,4-dimethylbenzoyl)-3,4-dihydro-1H-isoquinolin-7-yl]propanoate (114.00 mg, 218.08 umol, 36.54% yield, 95% purity). LCMS: Rt=1.62 min, m/z 497.3.

### 3. Preparation of 3-(1,4-dimethylbenzotriazol-5-yl)-3-[2-(3,4-dimethylbenzoyl)-3,4-dihydro-1H-isoquinolin-7-yl]propanoic acid

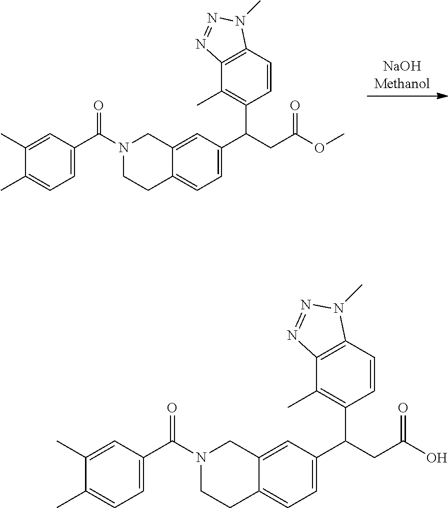

methyl 3-(1,4-dimethylbenzotriazol-5-yl)-3-[2-(3,4-dimethylbenzoyl)-3,4-dihydro-1H-isoquinolin-7-yl]propanoate (114.00 mg, 229.56 umol) in methanol (2.00 mL) was added NaOH (2 M, 229.56 uL) and microwaved at 100° C. for 30 min. After neutralization with 2M HCl, the crude was purified with prep HPLC to give 3-(1,4-dimethylbenzotriazol-5-yl)-3-[2-(3,4-dimethylbenzoyl)-3,4-dihydro-1H-isoquinolin-7-yl]propanoic acid (74.00 mg, 145.68 umol, 63.46% yield, 95% purity). LCMS: Rt=1.43 min, m/z=483.2. ¹H NMR (400 MHz, CHLOROFORM-d) δ 7.30-7.56 (m, 3H), 6.67-7.24 (m, 6H), 4.67-5.10 (m, 3H), 4.56 (br. s., 1H), 4.28 (br. s., 3H), 3.96 (br. s., 1H), 3.65 (br. s., 1H), 3.18 (d, J=12.55 Hz, 3H), 2.82 (br. s., 3H), 2.13-2.41 (m, 6H).

### Examples 100 and 101

### (3S)-3-(1,4-dimethylbenzotriazol-5-yl)-3-[2-(3,4-dimethylbenzoyl)-3,4-dihydro-1H-isoquinolin-7-yl]propanoic acid and (3R)-3-(1,4-dimethylbenzotriazol-5-yl)-3-[2-(3,4-dimethylbenzoyl)-3,4-dihydro-1H-isoquinolin-7-yl]propanoic acid

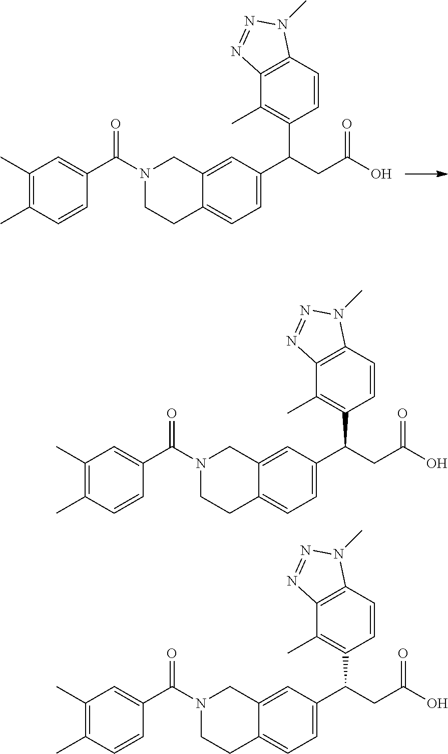

3-(1,4-dimethylbenzotriazol-5-yl)-3-[2-(3,4-dimethylbenzoyl)-3,4-dihydro-1H-isoquinolin-7-yl]propanoic acid (70.00 mg, 145.06 umol) was separated under SFC condition (Column: CHIRALCEL OD-H 30×250 mm, 5 um; Co-solvent: 25% Methanol in 0.1% DEA in CO₂ (flow rate: 100 mL/min), ABPR 120 bar, MBPR 40 psi) to give (3S)-3-(1,4-dimethylbenzotriazol-5-yl)-3-[2-(3,4-dimethylbenzoyl)-3,4-dihydro-1H-isoquinolin-7-yl]propanoic acid (20.60 mg, 35.22 umol, 12.14% yield, 95% purity, N-ethylethanamine). LCMS: Rt=1.42 min, m/z=483.2. ¹H NMR (400 MHz, CHLOROFORM-d) δ 7.45 (br. s., 1H), 6.83-7.26 (m, 7H), 2.68-5.31 (m, 15H), 2.55 (q, J=7.03 Hz, 8H), 2.11-2.38 (m, 6H), 1.01 (t, J=7.28 Hz, 12H).

and (3R)-3-(1,4-dimethylbenzotriazol-5-yl)-3-[2-(3,4-dimethylbenzoyl)-3,4-dihydro-1H-isoquinolin-7-yl]propanoic acid (22.20 mg, 37.95 umol, 13.08% yield, 95% purity, N-ethylethanamine). LCMS: Rt=1.42 min, m/z=483.2. ¹H NMR (400 MHz, CHLOROFORM-d) δ 7.45 (br. s., 1H), 6.75-7.23 (m, 7H), 2.69-5.20 (m, 15H), 2.46-2.65 (m, 8H), 2.05-2.35 (m, 6H), 1.02 (s, 12H).

### Examples 102 and 103

### (2R,3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoic acid and (2S,3S)-3-(1-ethyl-4-methyl-1H-benzo[d][1,2,3]triazol-5-yl)-2-methyl-3-(2-(2,3,5,6-tetramethylbenzoyl)-1,2,3,4-tetrahydroisoquinolin-7-yl)propanoic acid

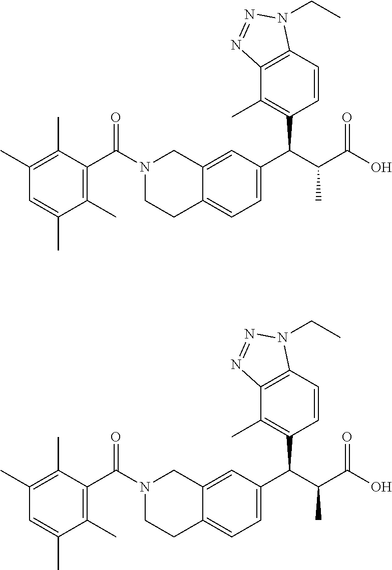 [SMILES: CCn1nnc2c(C)c([C@H](c3ccc4c(c3)CN(C(=O)c3c(C)c(C)cc(C)c3C)CC4)[C@@H](C)C(=O)O)ccc21 ; CCn1nnc2c(C)c([C@H](c3ccc4c(c3)CN(C(=O)c3c(C)c(C)cc(C)c3C)CC4)[C@H](C)C(=O)O)ccc21]

### 1. Preparation of tert-butyl 7-[(1S)-1-(1-ethyl-4-methyl-benzotriazol-5-yl)-3-methoxy-2-methyl-3-oxo-propyl]-3,4-dihydro-1H-isoquinoline-2-carboxylate

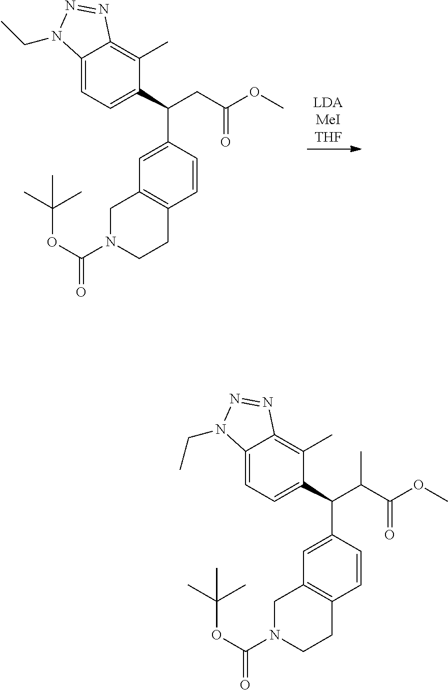

LDA (1 M, 250.74 uL) in THF (0.5 ml) was cooled to −78° C., and tert-butyl 7-[(1S)-1-(1-ethyl-4-methyl-benzotriazol-5-yl)-3-methoxy-3-oxo-propyl]-3,4-dihydro-1H-isoquinoline-2-carboxylate (100.00 mg, 208.95 umol) in THF (2.00 mL) was added and stirred for 2 h. then MeI (43.94 mg, 313.43 umol, 43.08 uL) was added and warmed to rt in 2 h. After quenched with water, the crude was extracted with EtOAc and dried and concentrated. The residue was purified with Si gel chromatography (HE/EA 0-100%) to give tert-butyl 7-[(1S)-1-(1-ethyl-4-methyl-benzotriazol-5-yl)-3-methoxy-2-methyl-3-oxo-propyl]-3,4-dihydro-1H-isoquinoline-2-carboxylate (77.00 mg, 148.49 umol, 71.07% yield, 95% purity). LCMS: Rt=1.87 min, m/z=493.2.

### 2. Preparation of tert-butyl 7-[(1S,2R)-1-(1-ethyl-4-methyl-benzotriazol-5-yl)-3-methoxy-2-methyl-3-oxo-propyl]-3,4-dihydro-1H-isoquinoline-2-carboxylate and tert-butyl 7-[(1S,2S)-1-(1-ethyl-4-methyl-benzotriazol-5-yl)-3-methoxy-2-methyl-3-oxo-propyl]-3,4-dihydro-1H-isoquinoline-2-carboxylate

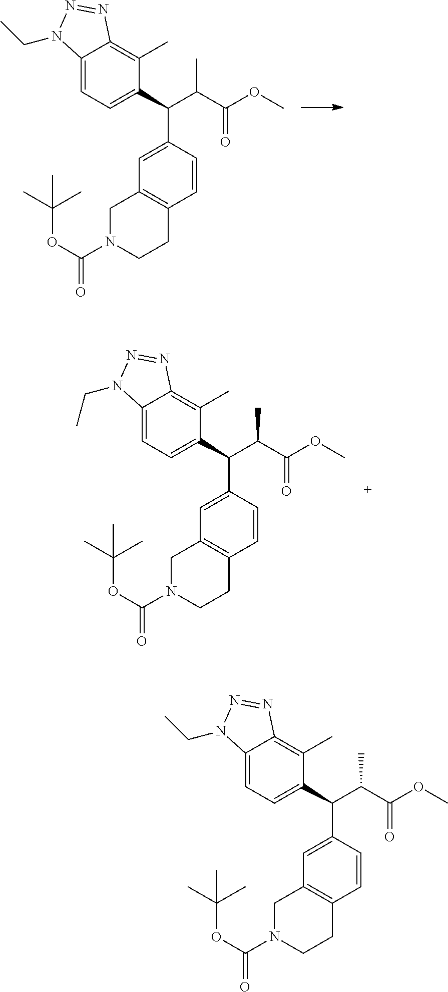

tert-butyl 7-[(1S)-1-(1-ethyl-4-methyl-benzotriazol-5-yl)-3-methoxy-2-methyl-3-oxo-propyl]-3,4-dihydro-1H-isoquinoline-2-carboxylate (366.00 mg, 742.98 umol) was separated with SFC condition: (Column: CHIRALPAK IC 30×250 mm, 5 um; Co-solvent: 30% Ethanol in 0.1% DEA in CO₂ (flow rate: 100 mL/min), ABPR 120 bar, MBPR 40 psi) to give tert-butyl 7-[(1S,2R)-1-(1-ethyl-4-methyl-benzotriazol-5-yl)-3-methoxy-2-methyl-3-oxo-propyl]-3,4-dihydro-1H-isoquinoline-2-carboxylate (68.80 mg, 132.68 umol, 17.86% yield, 95% purity). LCMS: Rt=1.84 min, m/z=493.3. And tert-butyl 7-[(1S,2S)-1-(1-ethyl-4-methyl-benzotriazol-5-yl)-3-methoxy-2-methyl-3-oxo-propyl]-3,4-dihydro-1H-isoquinoline-2-carboxylate (73.00 mg, 140.78 umol, 18.95% yield, 95% purity). LCMS: Rt=1.87 min, m/z=493.3.

### 3. Preparation of methyl (2R,3S)-3-(1-ethyl-4-methyl-1H-benzo[d][1,2,3]triazol-5-yl)-2-methyl-3-(1,2,3,4-tetrahydroisoquinolin-7-yl)propanoate

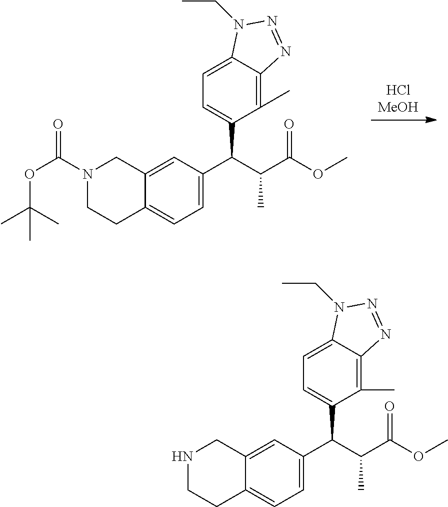 [SMILES: CCn1nnc2c(C)c([C@H](c3ccc4c(c3)CN(C(=O)OC(C)(C)C)CC4)[C@@H](C)C(=O)OC)ccc21 ; CCn1nnc2c(C)c([C@H](c3ccc4c(c3)CNCC4)[C@@H](C)C(=O)OC)ccc21 ; "HClMeOH"]

tert-butyl 7-[(1S,2R)-1-(1-ethyl-4-methyl-benzotriazol-5-yl)-3-methoxy-2-methyl-3-oxo-propyl]-3,4-dihydro-1H-isoquinoline-2-carboxylate (68.80 mg, 139.66 umol) in MeOH (2.00 mL) was added HCl (4 M, 69.83 uL) and stirred for overnight. LCMS: Rt=0.99 min, m/z=393.2. The crude was concentrated and used as is.

### 4. Preparation of methyl (2R,3S)-3-(1-ethyl-4-methyl-1H-benzo[d][1,2,3]triazol-5-yl)-2-methyl-3-(2-(2,3,5,6-tetramethylbenzoyl)-1,2,3,4-tetrahydroisoquinolin-7-yl)propanoate

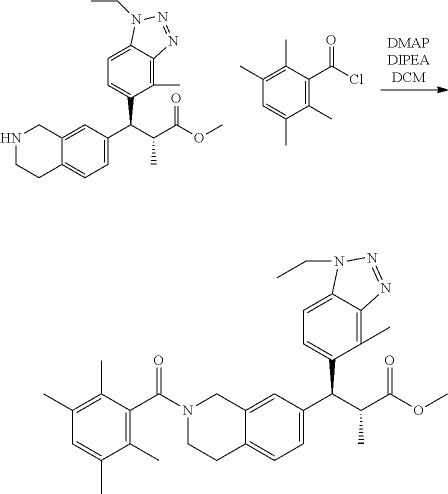

methyl (2R,3S)-3-(1-ethyl-4-methyl-1H-benzo[d][1,2,3]triazol-5-yl)-2-methyl-3-(1,2,3,4-tetrahydroisoquinolin-7-yl)propanoate (27.50 mg, 64.11 umol, Hydrochloride), 2,3,5,6-tetramethylbenzoyl chloride (18.91 mg, 96.17 umol), DMAP (783.21 ug, 6.41 umol), DIPEA (24.86 mg, 192.33 umol, 34.24 uL) in DCM (2.00 mL) was stirred at rt for overnight. The crude was chromatographed on Si gel (HE/EA 0-100%) to give methyl (2R,3S)-3-(1-ethyl-4-methyl-1H-benzo[d][1,2,3]triazol-5-yl)-2-methyl-3-(2-(2,3,5,6-tetramethylbenzoyl)-1,2,3,4-tetrahydroisoquinolin-7-yl)propanoate (32.20 mg, 58.26 umol, 90.87% yield). LCMS: Rt=1.71, 1.86 min. m/z=553.2.

### 5. Preparation of (2R,3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoic acid

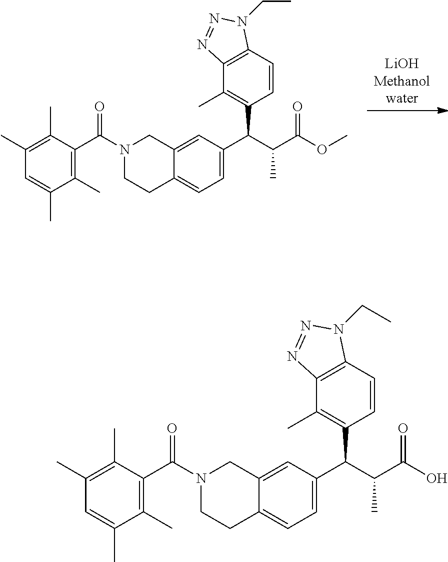

methyl (2R,3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoate (32.20 mg, 58.26 umol) and LiOH (2.79 mg, 116.52 umol) in methanol (1.00 mL), water (500.00 uL) and THF (1.00 mL) was microwaved at 100° C. for 30 min. After neutralization with 2M HCl, the crude was purified with prep HPLC to give (2R,3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoic acid (12.90 mg, 23.95 umol, 41.10% yield). LCMS: Rt=1.51, 1.64 min, m/z=539.3. ¹H NMR (400 MHz, CHLOROFORM-d) δ 7.30-7.71 (m, 2H), 6.65-7.25 (m, 4H), 2.55-5.32 (m, 13H), 1.86-2.29 (m, 12H), 1.44-1.73 (m, 3H), 0.89-1.30 (m, 3H).

### 6. Preparation of methyl (2S,3S)-3-(1-ethyl-4-methyl-benzotriazol-5-yl)-2-methyl-3-(1,2,3,4-tetrahydroisoquinolin-7-yl)propanoate

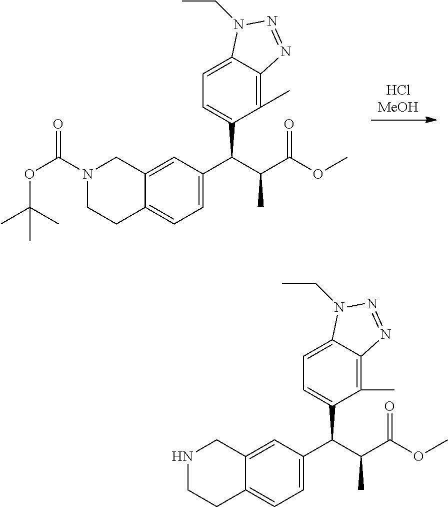 [SMILES: CCn1nnc2c(C)c([C@H](c3ccc4c(c3)CN(C(=O)OC(C)(C)C)CC4)[C@H](C)C(=O)OC)ccc21 ; CCn1nnc2c(C)c([C@H](c3ccc4c(c3)CNCC4)[C@H](C)C(=O)OC)ccc21 ; "HClMeOH"]

tert-butyl 7-[(1S,2S)-1-(1-ethyl-4-methyl-benzotriazol-5-yl)-3-methoxy-2-methyl-3-oxo-propyl]-3,4-dihydro-1H-isoquinoline-2-carboxylate (73.00 mg, 148.19 umol) in MeOH (2.00 mL) was added HCl (4 M, 74.10 uL) and stirred at rt for overnight. LCMS: Rt=0.93 min, m/z=393.2. The crude was used as is.

### 7. Preparation of methyl (2S,3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoate

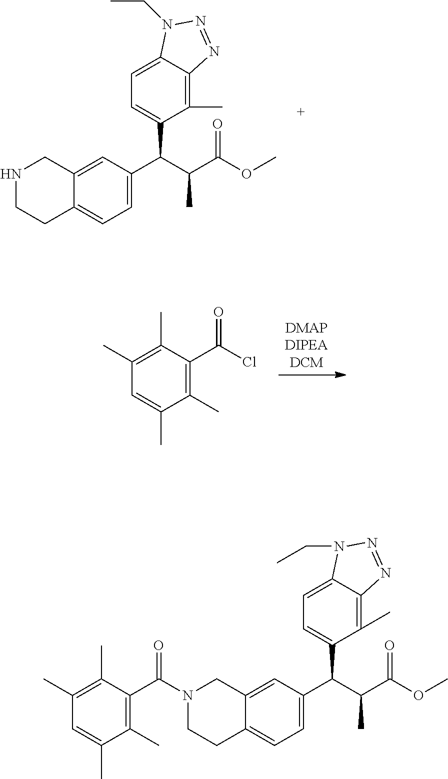

methyl (2S,3S)-3-(1-ethyl-4-methyl-benzotriazol-5-yl)-2-methyl-3-(1,2,3,4-tetrahydroisoquinolin-7-yl)propanoate (30.00 mg, 69.94 umol, Hydrochloride), 2,3,5,6-tetramethylbenzoyl chloride (20.63 mg, 104.91 umol), DMAP (854.46 ug, 6.99 umol), DIPEA (27.12 mg, 209.82 umol, 36.65 uL) in DCM (2.00 mL) was stirred at rt for overnight. The crude was purified on Si gel (HE/EA 0-100%) to give methyl (2S,3S)-3-(1-ethyl-4-methyl-benzotriazol-5-yl)-2-methyl-3-[2-(2,3,5,6-tetramethylbenzoyl)-3,4-dihydro -1H-isoquinolin-7-yl]propanoate (39.00 mg, 70.56 umol, 100.89% yield). LCMS: Rt=1.76, 1.92 min, m/z=553.3.

### 8. Preparation of (2S,3S)-3-(1-ethyl-4-methyl-1H-benzo[d][1,2,3]triazol-5-yl)-2-methyl-3-(2-(2,3,5,6-tetramethylbenzoyl)-1,2,3,4-tetrahydroisoquinolin-7-yl)propanoic acid

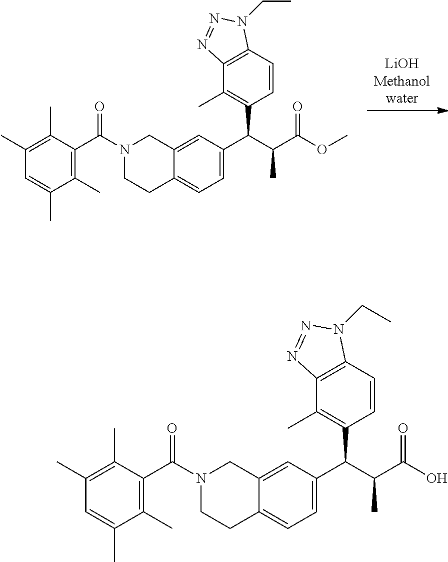

methyl (2S,3S)-3-(1-ethyl-4-methyl-1H-benzo[d][1,2,3]triazol-5-yl)-2-methyl-3-(2-(2,3,5,6-tetramethylbenzoyl)-1,2,3,4-tetrahydroisoquinolin-7-yl)propanoate (39.00 mg, 70.56 umol) and LiOH (3.38 mg, 141.12 umol) in methanol (1.00 mL), water (500.00 uL) and THF (1.00 mL) was microwaved at 100° C. for 50 min. After neutralization with 2M HCl, the crude was purified with prep HPLC to give (2S,3S)-3-(1-ethyl-4-methyl-1H-benzo[d][1,2,3]triazol-5-yl)-2-methyl-3-(2-(2,3,5,6-tetramethylbenzoyl)-1,2,3,4-tetrahydroisoquinolin-7-yl)propanoic acid (26.40 mg, 46.56 umol, 65.98% yield, 95% purity). LCMS: Rt=1.52, 1.73 min, m/z=539.3. ¹H NMR (400 MHz, CHLOROFORM-d) δ 7.30-7.76 (m, 2H), 6.67-7.22 (m, 4H), 2.63-5.28 (m, 13H), 1.82-2.37 (m, 12H), 1.59 (t, J=7.28 Hz, 3H), 1.11 (d, J=7.03 Hz, 3H).

### Examples 104 and 105

### (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoic acid and (2R,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoic acid

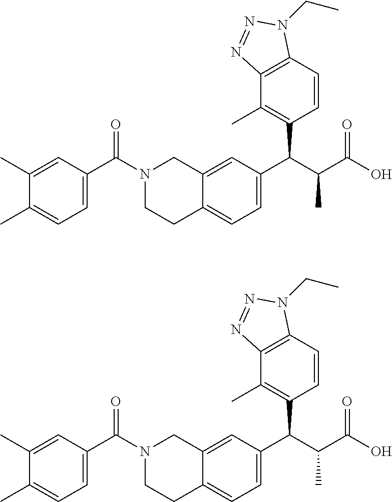 [SMILES: CCn1nnc2c(C)c([C@H](c3ccc4c(c3)CN(C(=O)c3ccc(C)c(C)c3)CC4)[C@H](C)C(=O)O)ccc21 ; CCn1nnc2c(C)c([C@H](c3ccc4c(c3)CN(C(=O)c3ccc(C)c(C)c3)CC4)[C@@H](C)C(=O)O)ccc21]

### 1. Preparation of methyl (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate

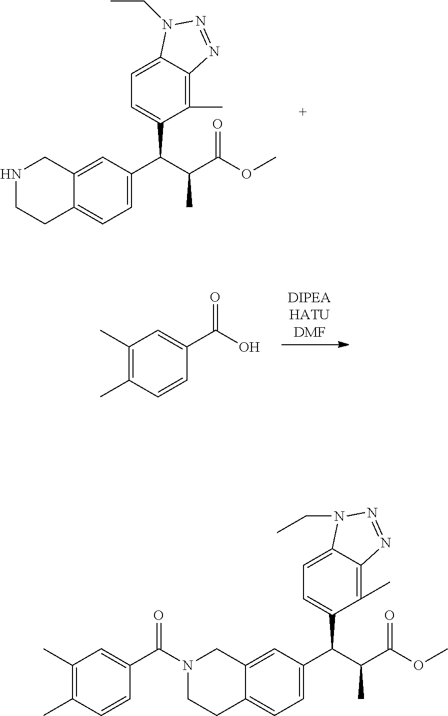

methyl (2S,3S)-3-(1-ethyl-4-methyl-benzotriazol-5-yl)-2-methyl-3-(1,2,3,4-tetrahydroisoquinolin-7-yl)propanoate (32.40 mg, 75.53 umol, Hydrochloride), 3,4-dimethylbenzoic acid (17.01 mg, 113.30 umol), DIPEA (29.29 mg, 226.59 umol, 39.58 uL) and HATU (43.19 mg, 113.30 umol) in DMF (2.00 mL) was stirred at rt for overnight. After dilution with EtOAc and washing with water, brine, the dried concentrated residue was purified with prep HPLC to give methyl (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate (17.00 mg, 30.78 umol, 40.76% yield, 95% purity). LCMS=1.77 min, m/z=525.3.

### 2. Preparation of (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoic acid

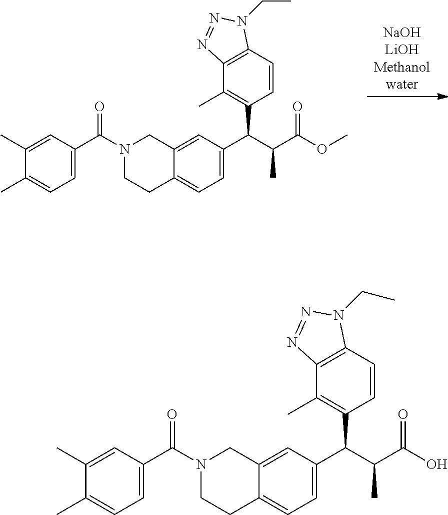

methyl (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate (17.00 mg, 32.40 umol) in methanol (749.98 uL) was added NaOH (2 M, 32.40 uL) and microwaved at 100° C. for 30 min. Only a small conversion. LiOH (1.55 mg, 64.80 umol) in water (150.08 uL) and THF (499.94 uL) was added and microwaved at 100° C. for 30 min. After neutralizing with 2M HCl, the crude was purified with prep HPLC to give (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoic acid (10.30 mg, 19.16 umol, 59.14% yield, 95% purity). LCMS: Rt=1.57 min, m/z=511.2. ¹H NMR (400 MHz, CHLOROFORM-d) δ 7.36-7.75 (m, 2H), 6.72-7.23 (m, 6H), 3.19-5.17 (m, 8H), 2.66-3.06 (m, 5H), 2.15-2.49 (m, 6H), 1.61 (br. s., 3H), 0.98-1.39 (m, 3H).

### 3. Preparation of methyl (2R,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate

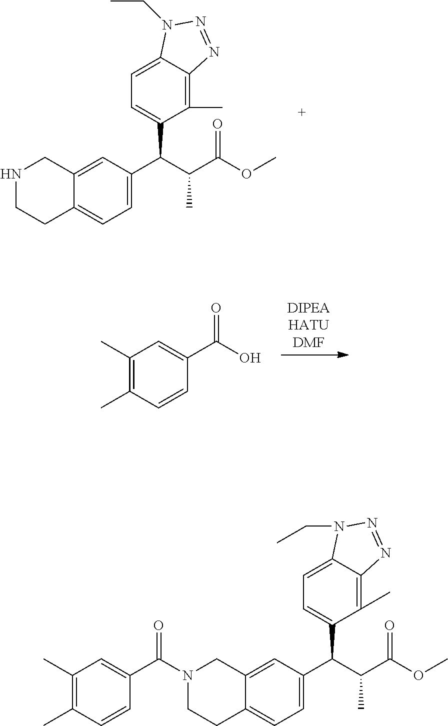

methyl (2R,3S)-3-(1-ethyl-4-methyl-benzotriazol-5-yl)-2-methyl-3-(1,2,3,4-tetrahydroisoquinolin-7-yl)propanoate (40.00 mg, 93.25 umol, Hydrochloride), 3,4-dimethylbenzoic acid (21.01 mg, 139.88 umol), HATU (53.33 mg, 139.88 umol) and DIPEA (36.15 mg, 279.75 umol, 48.85 uL) in DMF (2.00 mL) was stirred at rt for overnight. LCMS: Rt=1.75 min, m/z=525.2. After dilution with EtOAc and washing with water, the crude was purified with prep HPLC to give methyl (2R,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate (24.30 mg, 44.00 umol, 47.19% yield, 95% purity).

### 4. Preparation of (2R,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoic acid

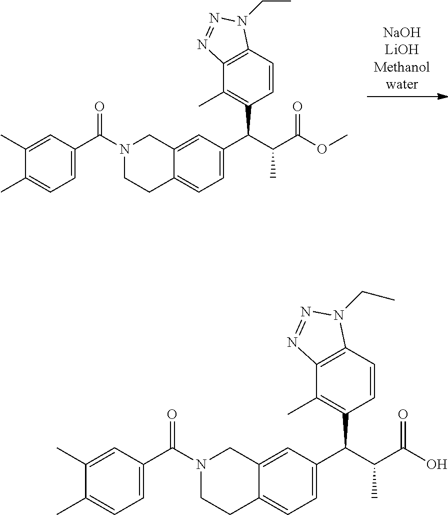

methyl (2R,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate (24.00 mg, 45.74 umol) in methanol (1.50 mL) was added NaOH (2 M, 45.74 uL) and microwaved at 100° C. for 30 min. No reaction. LiOH (2.19 mg, 91.48 umol) and water (199.98 uL), THF (500.07 uL) was added and microwaved again at 100° C. for 30 min. After neutralization with 2N HCl, the crude was purified with prep HPLC to give (2R,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoic acid (14.30 mg, 26.60 umol, 58.16% yield, 95% purity). LCMS: Rt=1.55 min min, m/z=511.2. ¹H NMR (400 MHz, CHLOROFORM-d) δ 7.39 (br. s., 1H), 6.29-7.23 (m, 7H), 3.15-5.17 (m, 8H), 2.68-3.03 (m, 5H), 2.16-2.48 (m, 6H), 1.62 (br. s., 3H), 0.97-1.37 (m, 3H).

### Example 106

### 3-(1-ethyl-4-methyl-benzotriazol-5-yl)-3-[2-(3,4,5-trimethylbenzoyl)-3,4-dihydro-1H-isoquinolin-7-yl]propanoic acid

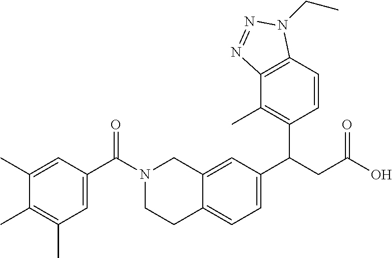 [SMILES: CCn1nnc2c(C)c(C(CC(=O)O)c3ccc4c(c3)CN(C(=O)c3cc(C)c(C)c(C)c3)CC4)ccc21]

### 1. Preparation of methyl 3-(1-ethyl-4-methyl-benzotriazol-5-yl)-3-[2-(3,4,5-trimethylbenzoyl)-3,4-dihydro-1H-isoquinolin-7-yl]propanoate

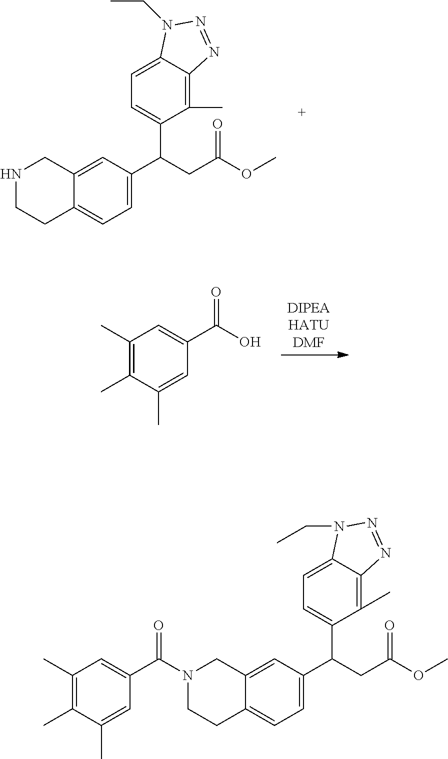

methyl 3-(1-ethyl-4-methyl-benzotriazol-5-yl)-3-(1,2,3,4-tetrahydroisoquinolin-7-yl)propanoate (200.00 mg, 528.44 umol), 3,4,5-trimethylbenzoic acid (104.12 mg, 634.13 umol), DIPEA (204.89 mg, 1.59 mmol, 276.88 uL), HATU (241.75 mg, 634.13 umol) in DMF (2.00 mL) was stirred at rt for overnight. The crude was diluted with EtOAc and washed with water 5 times and brine 1 time and dried over Na₂SO₄. After concentration, the residue was chromatographed on Si gel (HE/EA 0-100%) to give methyl 3-(1-ethyl-4-methyl-benzotriazol-5-yl)-3-[2-(3,4,5-trimethylbenzoyl)-3,4-dihydro-1H-isoquinolin-7-yl]propanoate (225.00 mg, 428.86 umol, 81.16% yield). LCMS: Rt=1.77 min, m/z=525.3.

### 2. Preparation of 3-(1-ethyl-4-methyl-benzotriazol-5-yl)-3-[2-(3,4,5-trimethylbenzoyl)-3,4-dihydro-1H-isoquinolin-7-yl]propanoic acid

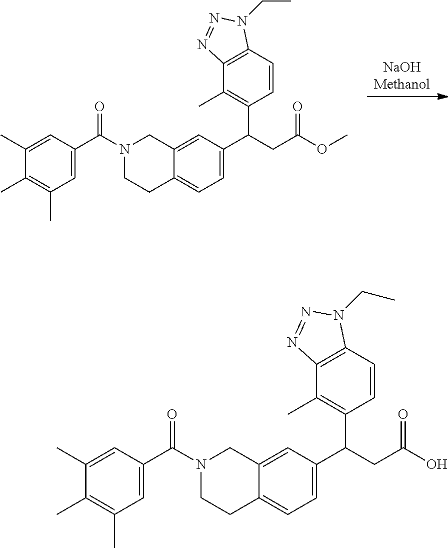

methyl 3-(1-ethyl-4-methyl-benzotriazol-5-yl)-3-[2-(3,4,5-trimethylbenzoyl)-3,4-dihydro-1H-isoquinolin-7-yl]propanoate (225.00 mg, 428.86 umol) in methanol (2.00 mL) was added NaOH (2 M, 428.86 uL) and microwaved at 100° C. for 30 min. After neutralization with 2M HCl, the crude was purified with prep HPLC to give 3-(1-ethyl-4-methyl-benzotriazol-5-yl)-3-[2-(3,4,5-trimethylbenzoyl)-3,4-dihydro-1H-isoquinolin-7-yl]propanoic acid (127.70 mg, 237.58 umol, 55.40% yield, 95% purity). LCMS: Rt=1.58 min, m/z=511.3. ¹H NMR (400 MHz, CHLOROFORM-d) δ 7.30-7.62 (m, 1H), 6.76-7.21 (m, 6H), 2.51-5.26 (m, 14H), 2.07-2.40 (m, 9H), 1.62 (t, J=6.90 Hz, 3H).

### Example 107

### (3S)-3-(1-ethyl-4-methyl-benzotriazol-5-yl)-3-[2-(3,4,5-trimethylbenzoyl)-3,4-dihydro-1H-isoquinolin-7-yl]propanoic acid

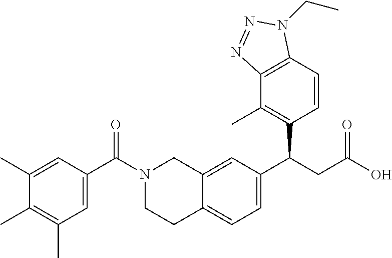 [SMILES: CCn1nnc2c(C)c([C@@H](CC(=O)O)c3ccc4c(c3)CN(C(=O)c3cc(C)c(C)c(C)c3)CC4)ccc21]

### 1. Preparation of methyl (3S)-3-(1-ethyl-4-methyl-benzotriazol-5-yl)-3-[2-(3,4,5-trimethylbenzoyl)-3,4-dihydro-1H-isoquinolin-7-yl]propanoate

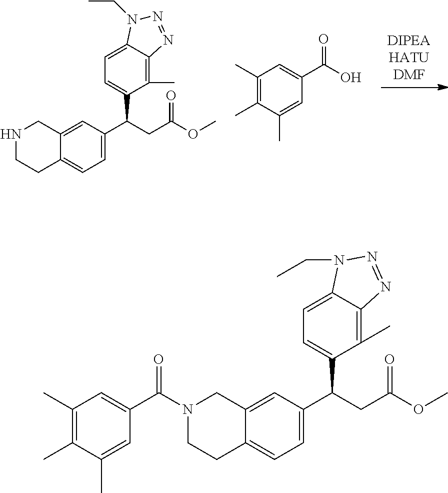

methyl (3S)-3-(1-ethyl-4-methyl-benzotriazol-5-yl)-3-(1,2,3,4-tetrahydroisoquinolin-7-yl)propanoate (500.00 mg, 1.21 mmol, Hydrochloride), 3,4,5-trimethylbenzoic acid (238.42 mg, 1.45 mmol), DIPEA (469.14 mg, 3.63 mmol, 633.97 uL), HATU (553.56 mg, 1.45 mmol) in DMF (3.00 mL) was stirred at rt for overnight. After dilution with EtOAc and washing with water and brine, the dried and concentrated residue was chromatographed on Si gel to give methyl (3S)-3-(1-ethyl-4-methyl-benzotriazol-5-yl)-3-[2-(3,4,5-trimethylbenzoyl)-3,4-dihydro-1H-isoquinolin-7-yl]propanoate (600.00 mg, 1.14 mmol, 94.51% yield). LCMS: Rt=1.78 min, m/z=525.3.

### 2. Preparation of (3S)-3-(1-ethyl-4-methyl-benzotriazol-5-yl)-3-[2-(3,4,5-trimethylbenzoyl)-3,4-dihydro-1H-isoquinolin-7-yl]propanoic acid

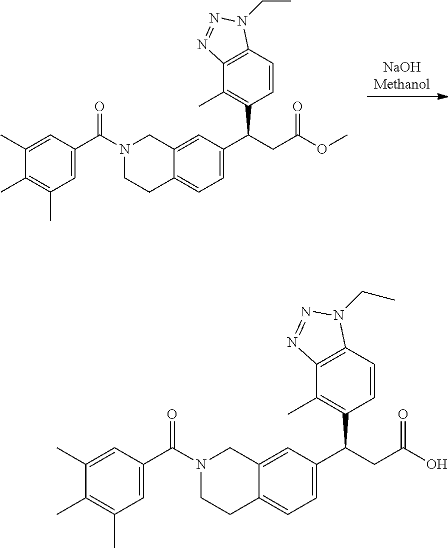

methyl (3S)-3-(1-ethyl-4-methyl-benzotriazol-5-yl)-3-[2-(3,4,5-trimethylbenzoyl)-3,4-dihydro-1H-isoquinolin-7-yl]propanoate (97.00 mg, 184.89 umol) in methanol (2.00 mL) was added NaOH (2 M, 184.89 uL) and microwaved at 100° C. for 30 min. After neutralization with 2M HCl, the crude was purified with prep HPLC to give (3S)-3-(1-ethyl-4-methyl-benzotriazol-5-yl)-3-[2-(3,4,5-trimethylbenzoyl)-3,4-dihydro-1H-isoquinolin-7-yl]propanoic acid (37.60 mg, 73.63 umol, 39.83% yield). LCMS: Rt=1.57 min, m/z=511.2. ¹H NMR (400 MHz, DMSO-d6) δ 7.38-7.79 (m, 2H), 6.73-7.32 (m, 5H), 4.80 (br. s., 1H), 2.92-4.71 (m, 8H), 2.76 (br. s., 5H), 2.24 (s, 6H), 2.14 (s, 3H), 1.46 (t, J=7.28 Hz, 3H).

### Examples 108 and 109

### (2R,3S)-3-(1-ethyl-4-methyl-benzotriazol-5-yl)-2-methyl-3-[2-(3,4,5-trimethylbenzoyl)-3,4-dihydro-1H-isoquinolin-7-yl]propanoic acid and (2S,3S)-3-(1-ethyl-4-methyl-benzotriazol-5-yl)-2-methyl-3-[2-(3,4,5-trimethylbenzoyl)-3,4-dihydro-1H-isoquinolin-7-yl]propanoic acid

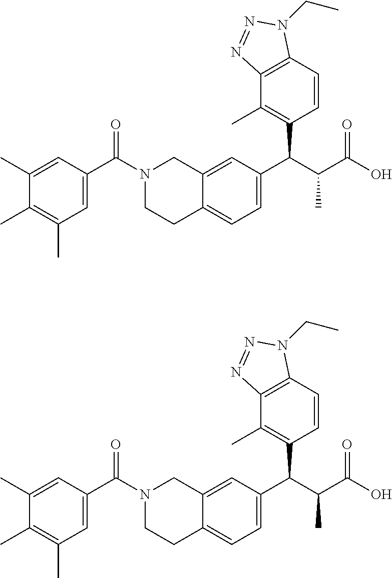 [SMILES: CCn1nnc2c(C)c([C@H](c3ccc4c(c3)CN(C(=O)c3cc(C)c(C)c(C)c3)CC4)[C@@H](C)C(=O)O)ccc21 ; CCn1nnc2c(C)c([C@H](c3ccc4c(c3)CN(C(=O)c3cc(C)c(C)c(C)c3)CC4)[C@H](C)C(=O)O)ccc21]

### 1. Preparation of methyl (3S)-3-(1-ethyl-4-methyl-benzotriazol-5-yl)-2-methyl-3-[2-(3,4,5-trimethylbenzoyl)-3,4-dihydro-1H-isoquinolin-7-yl]propanoate

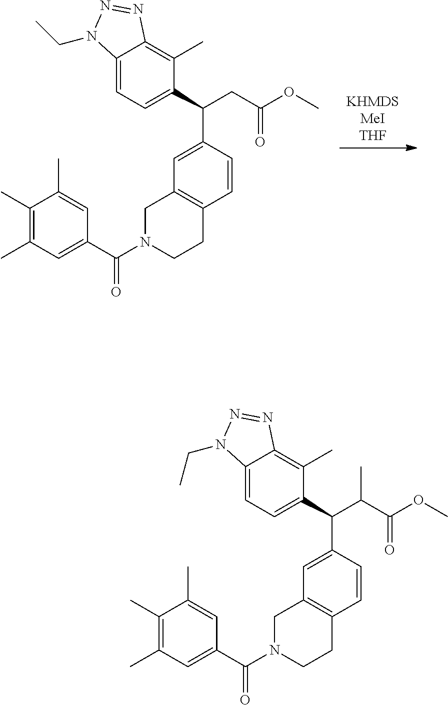

KHMDS (1 M, 386.54 uL) in THF (2.00 mL) was cooled to −78° C., methyl (3S)-3-(1-ethyl-4-methyl-benzotriazol-5-yl)-3-[2-(3,4,5-trimethylbenzoyl)-3,4-dihydro-1H-isoquinolin-7-yl]propanoate (169.00 mg, 322.12 umol) in THF (2.00 mL) was added dropwise, and warmed up to −20° C. for 2 h. MeI (67.73 mg, 483.18 umol, 66.40 uL) was added and stirred at rt for overnight. After quenching with water and extracting with EtOAc, the dried concentrated residue was chromatographed on Si gel (HE/EA 0-100%) to give methyl (3S)-3-(1-ethyl-4-methyl-benzotriazol-5-yl)-2-methyl-3-[2-(3,4,5-trimethylbenzoyl)-3,4-dihydro-1H-isoquinolin-7-yl]propanoate (163.50 mg, 303.52 umol, 94.23% yield). LCMS: Rt=1.84, 1.91 min, m/z=539.3.

### 2. Preparation of (2R,3S)-3-(1-ethyl-4-methyl-benzotriazol-5-yl)-2-methyl-3-[2-(3,4,5-trimethylbenzoyl)-3,4-dihydro-1H-isoquinolin-7-yl]propanoic acid and (2S,3S)-3-(1-ethyl-4-methyl-benzotriazol-5-yl)-2-methyl-3-[2-(3,4,5-trimethylbenzoyl)-3,4-dihydro-1H-isoquinolin-7-yl]propanoic acid

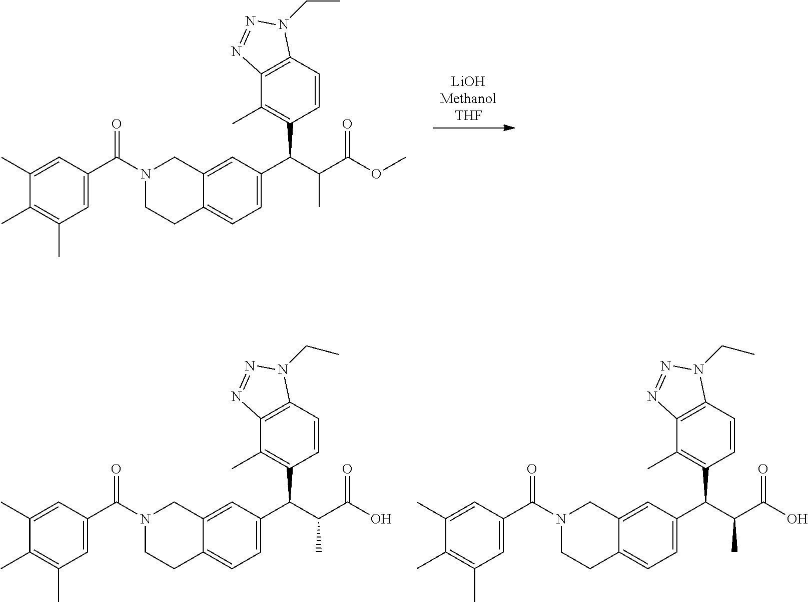

methyl (3S)-3-(1-ethyl-4-methyl-benzotriazol-5-yl)-2-methyl-3-[2-(3,4,5-trimethylbenzoyl)-3,4-dihydro-1H-isoquinolin-7-yl]propanoate (163.50 mg, 303.52 umol) in methanol (2.00 mL), THF (1000.00 uL), water (500.17 uL) was added LiOH (14.54 mg, 607.04 umol) and microwaved at 100° C. for 50 min. After neutralization with 2M HCl, the crude was purified with prep HPLC to give (2R,3S)-3-(1-ethyl-4-methyl-benzotriazol-5-yl)-2-methyl-3-[2-(3,4,5-trimethylbenzoyl)-3,4-dihydro-1H-isoquinolin-7-yl]propanoic acid (37.70 mg, 68.26 umol, 22.49% yield, 95% purity). as Peak 1: LCMS: Rt=1.59, 1.63 min, m/z=525.3. ¹H NMR (400 MHz, CHLOROFORM-d) δ 7.30-7.50 (m, 2H), 6.90-7.24 (m, 5H), 3.22-5.12 (m, 8H), 2.56-3.05 (m, 5H), 2.06-2.40 (m, 9H), 1.62 (t, J=6.78 Hz, 3H), 1.15 (br. s., 3H).

And (2S,3S)-3-(1-ethyl-4-methyl-benzotriazol-5-yl)-2-methyl-3-[2-(3,4,5-trimethylbenzoyl)-3,4-dihydro-1H-isoquinolin-7-yl]propanoic acid (36.00 mg, 65.19 umol, 21.48% yield, 95% purity) as Peak 2: LCMS: Rt=1.63 min, m/z 525.3. ¹H NMR (400 MHz, CHLOROFORM-d) δ 7.31-7.80 (m, 2H), 6.88-7.21 (m, 5H), 3.03-5.03 (m, 8H), 2.80 (br. s., 5H), 2.12-2.43 (m, 9H), 1.60 (t, J=7.03 Hz, 3H), 1.25 (br. s., 3H).

### Examples 110 and 111

### (3S)-3-[2-(3,4-dimethylbenzoyl)-3,4-dihydro-1H-isoquinolin-7-yl]-3-(1-ethyl-4-methyl-benzotriazol-5-yl)propanoic acid and (3R)-3-[2-(3,4-dimethylbenzoyl)-3,4-dihydro-1H-isoquinolin-7-yl]-3-(1-ethyl-4-methyl-benzotriazol-5-yl)propanoic acid

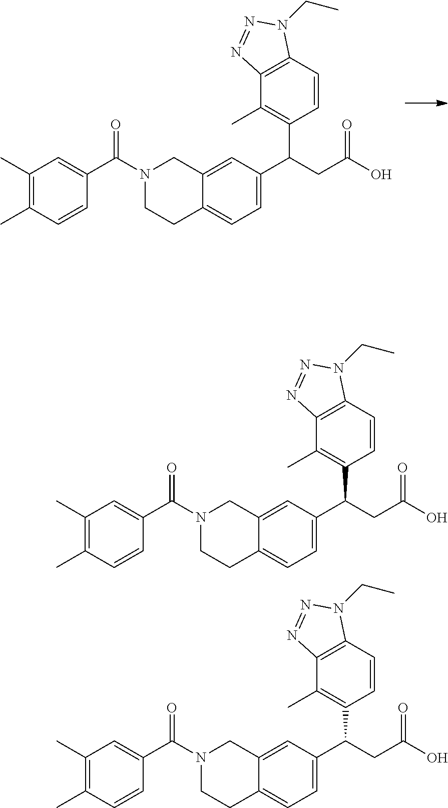

3-[2-(3,4-dimethylbenzoyl)-3,4-dihydro-1H-isoquinolin-7-yl]-3-(1-ethyl-4-methyl-benzotriazol-5-yl)propanoic acid (106.00 mg, 213.45 umol) was separated under SFC condition: (Column: 2.1×25.0 cm Chiralcel OX-H from Chiral Technologies (West Chester, Pa.); CO₂ Co-solvent: Ethanol with 0.25% Isopropylamine; Isocratic Method: 60% Co-solvent at 60 g/min; System pressure: 125 bar; Sample diluent: Ethanol.) to give (3S)-3-[2-(3,4-dimethylbenzoyl)-3,4-dihydro-1H-isoquinolin-7-yl]-3-(1-ethyl-4-methyl-benzotriazol-5-yl)propanoic acid (17.50 mg, 35.24 umol, 16.51% yield, 100% purity) as peak 2 and re-purified with TFA buffered ACN/water. LCMS: Rt=1.51 min, m/z=497.2. ¹H NMR (400 MHz, CHLOROFORM-d) δ 7.39 (br. s., 2H), 6.90-7.24 (m, 6H), 2.87-5.43 (m, 11H), 2.82 (br. s., 3H), 2.12-2.42 (m, 6H), 1.61 (br. s., 3H).

and Peak1 as (3R)-3-[2-(3,4-dimethylbenzoyl)-3,4-dihydro-1H-isoquinolin-7-yl]-3-(1-ethyl-4-methyl-benzotriazol-5-yl)propanoic acid (34.00 mg, 59.68 umol, 27.96% yield, 100% purity, N-ethylethanamine). LCMS: Rt=1.51 min, m/z=497.2. ¹H NMR (400 MHz, CHLOROFORM-d) δ 6.67-7.61 (m, 8H), 2.91-5.59 (m, 11H), 2.83 (br. s., 3H), 2.20-2.36 (m, 6H), 1.63 (t, J=6.90 Hz, 3H).

### Example 112

### 3-[2-(4-allylbenzoyl)-3,4-dihydro-1H-isoquinolin-7-yl]-3-(1-ethyl-4-methyl-benzotriazol-5-yl)propanoic acid

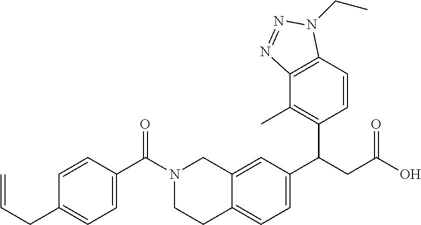 [SMILES: C=CCc1ccc(C(=O)N2CCc3ccc(C(CC(=O)O)c4ccc5c(nnn5CC)c4C)cc3C2)cc1]

3-[2-(4-allylbenzoyl)-3,4-dihydro-1H-isoquinolin-7-yl]-3-(1-ethyl-4-methyl-benzotriazol-5-yl)propanoic acid (176.90 mg, 330.42 umol, 81.84% yield, 95% purity) was made following the general procedure as Example 1. LCMS: Rt=1.57 min, m/z=509.2. ¹H NMR (400 MHz, CHLOROFORM-d) δ 7.29-7.56 (m, 6H), 6.90-7.25 (m, 3H), 5.71-6.56 (m, 2H), 2.51-5.30 (m, 15H), 1.92 (d, J=6.27 Hz, 2H), 1.62 (t, J=6.65 Hz, 3H).

### Example 113

### 3-(1-ethyl-4-methyl-1H-benzo[d][1,2,3]triazol-5-yl)-3-(2-(2,4,5-trimethylbenzoyl)-1,2,3,4-tetrahydroisoquinolin-7-yl)propanoic acid

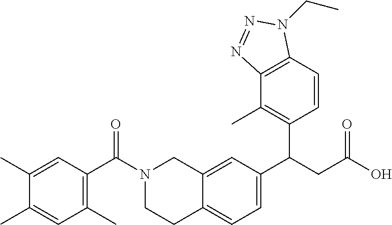 [SMILES: CCn1nnc2c(C)c(C(CC(=O)O)c3ccc4c(c3)CN(C(=O)c3cc(C)c(C)cc3C)CC4)ccc21]

3-(1-ethyl-4-methyl-1H-benzo[d][1,2,3]triazol-5-yl)-3-(2-(2,4,5-trimethylbenzoyl)-1,2,3,4-tetrahydroisoquinolin-7-yl)propanoic acid (147.00 mg, 273.49 umol, 73.21% yield, 95% purity) was made following the general procedure as Example 1. LCMS: Rt=1.55 min, m/z=511.3. ¹H NMR (400 MHz, CHLOROFORM-d) δ 7.29-7.52 (m, 2H), 6.65-7.19 (m, 5H), 4.84-5.11 (m, 1H), 4.55-4.77 (m, 2H), 2.58-4.45 (m, 11H), 1.95-2.38 (m, 9H), 1.49-1.78 (m, 3H).

### Example 114

### (3S)-3-(1-ethyl-4-methyl-benzotriazol-5-yl)-3-[2-(2,4,5-trimethylbenzoyl)-3,4-dihydro-1H-isoquinolin-7-yl]propanoic acid

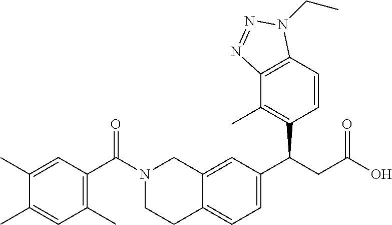 [SMILES: CCn1nnc2c(C)c([C@@H](CC(=O)O)c3ccc4c(c3)CN(C(=O)c3cc(C)c(C)cc3C)CC4)ccc21]

(3S)-3-(1-ethyl-4-methyl-benzotriazol-5-yl)-3-[2-(2,4,5-trimethylbenzoyl)-3,4-dihydro-1H-isoquinolin-7-yl]propanoic acid (68.70 mg, 127.81 umol, 66.85% yield, 95% purity) was made following the general procedure as Examples 107. LCMS: Rt=1.56 min, m/z=511.2. ¹H NMR (400 MHz, CHLOROFORM-d) δ 9.29 (br. s., 1H), 7.28-7.47 (m, 2H), 6.55-7.18 (m, 5H), 2.87-5.39 (m, 10H), 2.64-2.83 (m, 4H), 1.94-2.41 (m, 9H), 1.44-1.74 (m, 3H).

### Examples 115 and 116

### (3S)-3-[2-(2,6-diethylbenzoyl)-3,4-dihydro-1H-isoquinolin-7-yl]-3-(1-ethyl-4-methyl-benzotriazol-5-yl)propanoic acid and (3R)-3-[2-(2,6-diethylbenzoyl)-3,4-dihydro-1H-isoquinolin-7-yl]-3-(1-ethyl-4-methyl-benzotriazol-5-yl)propanoic acid

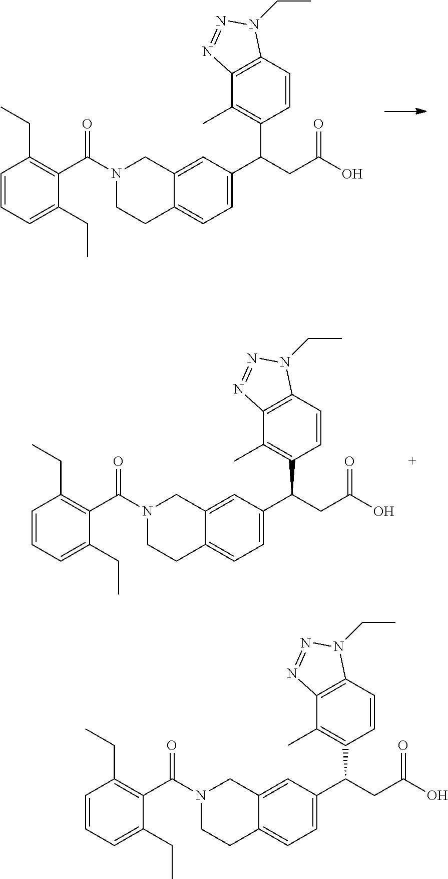 [SMILES: CCc1cccc(CC)c1C(=O)N1CCc2ccc(C(CC(=O)O)c3ccc4c(nnn4CC)c3C)cc2C1 ; CCc1cccc(CC)c1C(=O)N1CCc2ccc([C@H](CC(=O)O)c3ccc4c(nnn4CC)c3C)cc2C1 ; CCc1cccc(CC)c1C(=O)N1CCc2ccc([C@@H](CC(=O)O)c3ccc4c(nnn4CC)c3C)cc2C1]

3-[2-(2,6-diethylbenzoyl)-3,4-dihydro-1H-isoquinolin-7-yl]-3-(1-ethyl-4-methyl-benzotriazol-5-yl)propanoic acid (79.00 mg, 150.58 umol) was separated under SFC condition (Column: CHIRALPAK IC 30×250 mm, 5 um; Co-solvent: 30% Ethanol in 0.1% DEA in CO2 (flow rate: 100 mL/min), ABPR 120 bar, MBPR 40 psi) to give peak 1 (3S)-3-[2-(2,6-diethylbenzoyl)-3,4-dihydro-1H-isoquinolin-7-yl]-3-(1-ethyl-4-methyl-benzotriazol-5-yl)propanoic acid (24.00 mg, 38.14 umol, 50.66% yield, 95% purity, N-ethylethanamine). LCMS: Rt=1.49, 1.68 min, m/z 525.3. ¹H NMR (400 MHz, CHLOROFORM-d) δ 7.29-7.61 (m, 2H), 6.40-7.25 (m, 6H), 2.29-5.34 (m, 15H), 1.46-1.73 (m, 3H), 1.22 (br. s., 3H), 1.09 (d, J=4.77 Hz, 3H), 0.90 (br. s., 3H).

and peak 2 (3R)-3-[2-(2,6-diethylbenzoyl)-3,4-dihydro-1H-isoquinolin-7-yl]-3-(1-ethyl-4-methyl-benzotriazol-5-yl)propanoic acid (22.00 mg, 34.96 umol, 46.43% yield, 95% purity, N-ethylethanamine). LCMS: Rt=1.49, 1.68 min, m/z 525.3. ¹H NMR (400 MHz, CHLOROFORM-d) δ 7.41 (s, 2H), 6.41-7.19 (m, 6H), 2.20-5.24 (m, 15H), 1.45-1.77 (m, 3H), 1.14-1.34 (m, 3H), 1.03-1.12 (m, 3H), 0.91 (t, J=7.28 Hz, 3H).

### Examples 117 and 118

### (3S)-3-(1-ethyl-4-methyl-benzotriazol-5-yl)-3-[2-(2,3,5-trimethylbenzoyl)-3,4-dihydro-1H-isoquinolin-7-yl]propanoic acid and (3R)-3-(1-ethyl-4-methyl-benzotriazol-5-yl)-3-[2-(2,3,5-trimethylbenzoyl)-3,4-dihydro-1H-isoquinolin-7-yl]propanoic acid

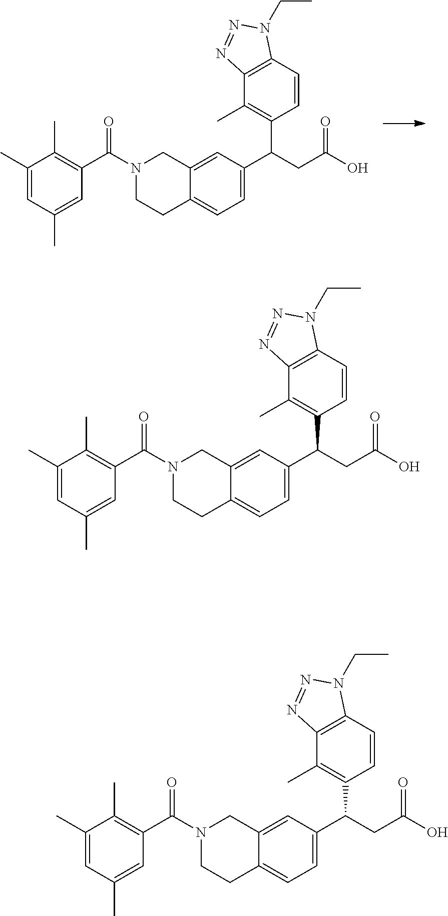 [SMILES: CCn1nnc2c(C)c(C(CC(=O)O)c3ccc4c(c3)CN(C(=O)c3cc(C)cc(C)c3C)CC4)ccc21 ; CCn1nnc2c(C)c([C@@H](CC(=O)O)c3ccc4c(c3)CN(C(=O)c3cc(C)cc(C)c3C)CC4)ccc21 ; CCn1nnc2c(C)c([C@H](CC(=O)O)c3ccc4c(c3)CN(C(=O)c3cc(C)cc(C)c3C)CC4)ccc21]

3-(1-ethyl-4-methyl-benzotriazol-5-yl)-3-[2-(2,3,5-trimethylbenzoyl)-3,4-dihydro-1H-isoquinolin-7-yl]propanoic acid (130.00 mg, 254.59 umol) was separated under the SFC condition (Column: CHIRALCEL OD-H 30×250 mm, 5 um; Co-solvent: 20% Methanol in 0.1% DEA in CO₂ (flow rate: 100 mL/min), ABPR 120 bar, MBPR 40 psi) to give Peak 1 (3S)-3-(1-ethyl-4-methyl-benzotriazol-5-yl)-3-[2-(2,3,5-trimethylbenzoyl)-3,4-dihydro-1H-isoquinolin-7-yl]propanoic acid (35.20 mg, 57.28 umol, 22.50% yield, 95% purity, N-ethylethanamine). LCMS: Rt=1.56 min, m/z=511.2. ¹H NMR (400 MHz, CHLOROFORM-d) δ 7.29-7.74 (m, 2H), 6.52-7.19 (m, 5H), 2.63-5.29 (m, 14H), 2.55 (d, J=7.28 Hz, 4H), 1.92-2.36 (m, 9H), 1.58 (d, J=8.78 Hz, 3H), 0.92 (br. s., 6H).

and peak 2 (3R)-3-(1-ethyl-4-methyl-benzotriazol-5-yl)-3-[2-(2,3,5-trimethylbenzoyl)-3,4-dihydro-1H-isoquinolin-7-yl]propanoic acid (32.40 mg, 52.73 umol, 20.71% yield, 95% purity, N-ethylethanamine). LCMS: RT=1.56 min, m/z=511.2. ¹H NMR (400 MHz, CHLOROFORM-d) δ 7.32 (br. s., 2H), 6.36-7.16 (m, 5H), 2.66-5.44 (m, 14H), 2.55 (br. s., 4H), 1.92-2.36 (m, 9H), 1.41-1.70 (m, 3H), 0.93 (t, J=6.65 Hz, 6H).

### Examples 119 and 120

### (S)-3-(2-(2,5-dimethylbenzoyl)-1,2,3,4-tetrahydroisoquinolin-7-yl)-3-(1-ethyl-4-methyl-1H-benzo[d][1,2,3]triazol-5-yl)propanoic acid and (R)-3-(2-(2,5-dimethylbenzoyl)-1,2,3,4-tetrahydroisoquinolin-7-yl)-3-(1-ethyl-4-methyl-1H-benzo[d][1,2,3]triazol-5-yl)propanoic acid

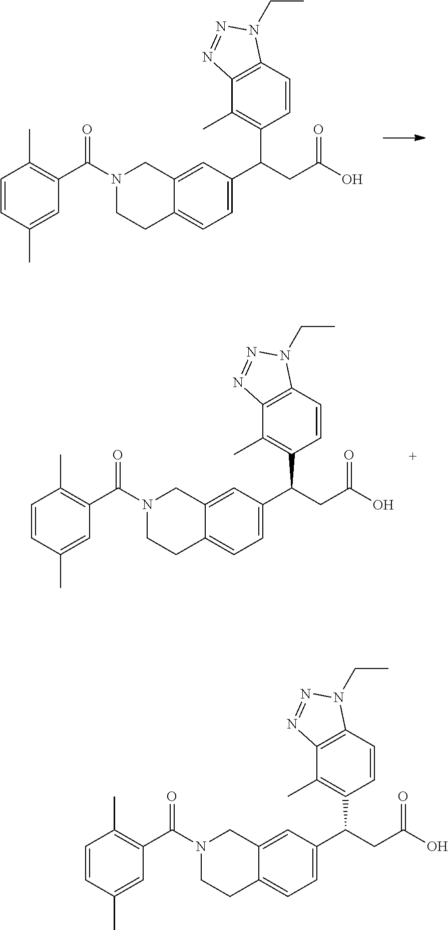 [SMILES: CCn1nnc2c(C)c(C(CC(=O)O)c3ccc4c(c3)CN(C(=O)c3cc(C)ccc3C)CC4)ccc21 ; CCn1nnc2c(C)c([C@@H](CC(=O)O)c3ccc4c(c3)CN(C(=O)c3cc(C)ccc3C)CC4)ccc21 ; CCn1nnc2c(C)c([C@H](CC(=O)O)c3ccc4c(c3)CN(C(=O)c3cc(C)ccc3C)CC4)ccc21]

3-(2-(2,5-dimethylbenzoyl)-1,2,3,4-tetrahydroisoquinolin-7-yl)-3-(1-ethyl-4-methyl-1H-benzo[d][1,2,3]triazol-5-yl)propanoic acid (16.40 mg, 33.02 umol) was separated under the SFC condition (Column: CHIRALCEL AD-H 30×250 mm, 5 um; Co-solvent: 40% Ethanol in 0.1% DEA in CO₂ (flow rate: 100 mL/min), ABPR 120 bar, MBPR 40 psi) to give peak 1 (S)-3-(2-(2,5-dimethylbenzoyl)-1,2,3,4-tetrahydroisoquinolin-7-yl)-3-(1-ethyl-4-methyl-1H-benzo[d][1,2,3]triazol-5-yl)propanoic acid (5.60 mg, 9.34 umol, 28.28% yield, 95% purity, N-ethylethanamine). LCMS: Rt=1.48 min, m/z=497.2. ¹H NMR (400 MHz, CHLOROFORM-d) δ 6.50-7.59 (m, 8H), 2.64-5.17 (m, 14H), 2.49 (br. s., 4H), 1.94-2.38 (m, 6H), 1.55 (d, J=6.78 Hz, 3H), 0.90 (t, J=7.03 Hz, 6H).

and peak 2 (R)-3-(2-(2,5-dimethylbenzoyl)-1,2,3,4-tetrahydroisoquinolin-7-yl)-3-(1-ethyl-4-methyl-1H-benzo[d][1,2,3]triazol-5-yl)propanoic acid (5.00 mg, 8.34 umol, 25.25% yield, 95% purity, N-ethylethanamine). LCMS: Rt=1.48 min, m/z=497.2. ¹H NMR (400 MHz, CHLOROFORM-d) δ 6.51-7.52 (m, 8H), 2.64-5.34 (m, 14H), 2.51 (br. s., 4H), 1.94-2.39 (m, 6H), 1.55 (d, J=6.27 Hz, 3H), 0.90 (t, J=7.03 Hz, 6H).

### Examples 121 and 122

### (S)-3-(1-ethyl-4-methyl-1H-benzo[d][1,2,3]triazol-5-yl)-3-(2-(2-fluoro-5-methylbenzoyl)-1,2,3,4-tetrahydroisoquinolin-7-yl)propanoic acid and (R)-3-(1-ethyl-4-methyl-1H-benzo[d][1,2,3]triazol-5-yl)-3-(2-(2-fluoro-5-methylbenzoyl)-1,2,3,4-tetrahydroisoquinolin-7-yl)propanoic acid

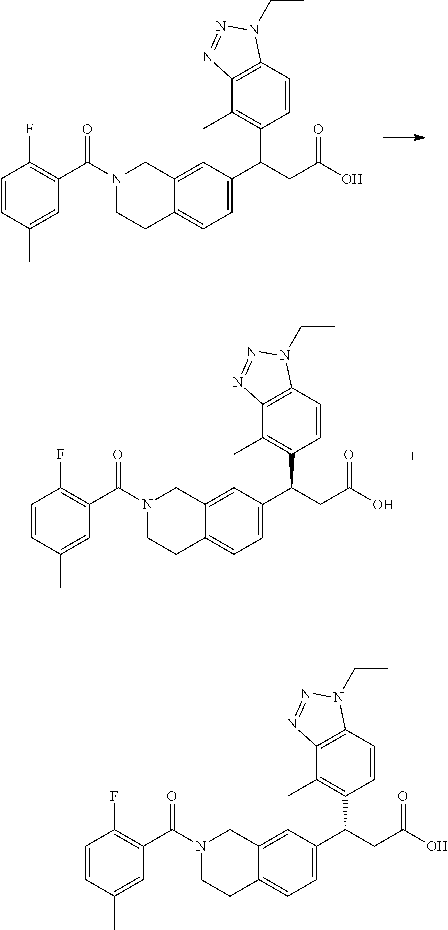 [SMILES: CCn1nnc2c(C)c(C(CC(=O)O)c3ccc4c(c3)CN(C(=O)c3cc(C)ccc3F)CC4)ccc21 ; CCn1nnc2c(C)c([C@@H](CC(=O)O)c3ccc4c(c3)CN(C(=O)c3cc(C)ccc3F)CC4)ccc21 ; CCn1nnc2c(C)c([C@H](CC(=O)O)c3ccc4c(c3)CN(C(=O)c3cc(C)ccc3F)CC4)ccc21]

3-(1-ethyl-4-methyl-1H-benzo[d][1,2,3]triazol-5-yl)-3-(2-(2-fluoro-5-methylbenzoyl)-1,2,3,4-tetrahydroisoquinolin-7-yl)propanoic acid (22.00 mg, 43.95 umol) was separated under the following SFC condition (Column: CHIRALCEL AD-H 30×250 mm, 5 um; Co-solvent: 40% Ethanol in 0.1% DEA in CO₂ (flow rate: 100 mL/min), ABPR 120 bar, MBPR 40 psi) to give peak 1 (S)-3-(1-ethyl-4-methyl-1H-benzo[d][1,2,3]triazol-5-yl)-3-(2-(2-fluoro-5-methylbenzoyl)-1,2,3,4-tetrahydroisoquinolin-7-yl)propanoic acid (7.20 mg, 11.92 umol, 27.13% yield, 95% purity, N-ethylethanamine). LCMS: Rt=1.45 min, m/z=501.2. ¹H NMR (400 MHz, CHLOROFORM-d) δ 6.62-7.64 (m, 8H), 2.63-5.13 (m, 14H), 2.48 (d, J=6.78 Hz, 4H), 2.23-2.37 (m, 3H), 1.55 (q, J=7.19 Hz, 3H), 0.90 (t, J=7.15 Hz, 6H).

and Peak 2 (R)-3-(1-ethyl-4-methyl-1H-benzo[d][1,2,3]triazol-5-yl)-3-(2-(2-fluoro-5-methylbenzoyl)-1,2,3,4-tetrahydroisoquinolin-7-yl)propanoic acid (6.50 mg, 10.76 umol, 24.49% yield, 95% purity, N-ethylethanamine). LCMS: Rt=1.45 min, m/z=501.2. ¹H NMR (400 MHz, CHLOROFORM-d) δ 6.67-7.64 (m, 8H), 2.67-5.07 (m, 14H), 2.49 (br. s., 4H), 2.31 (d, J=8.53 Hz, 3H), 1.55 (d, J=7.03 Hz, 3H), 0.91 (t, J=7.15 Hz, 6H).

### Examples 123 and 124

### (S)-3-(2-(3,4-dimethylbenzoyl)-1,2,3,4-tetrahydroisoquinolin-7-yl)-3-(1-ethyl-4-methyl-1H-benzo[d][1,2,3]triazol-5-yl)-N-hydroxy-N-methylpropanamide and methylamino (3S)-3-[2-(3,4-dimethylbenzoyl)-3,4-dihydro-1H-isoquinolin-7-yl]-3-(1-ethyl-4-methyl-benzotriazol-5-yl)propanoate

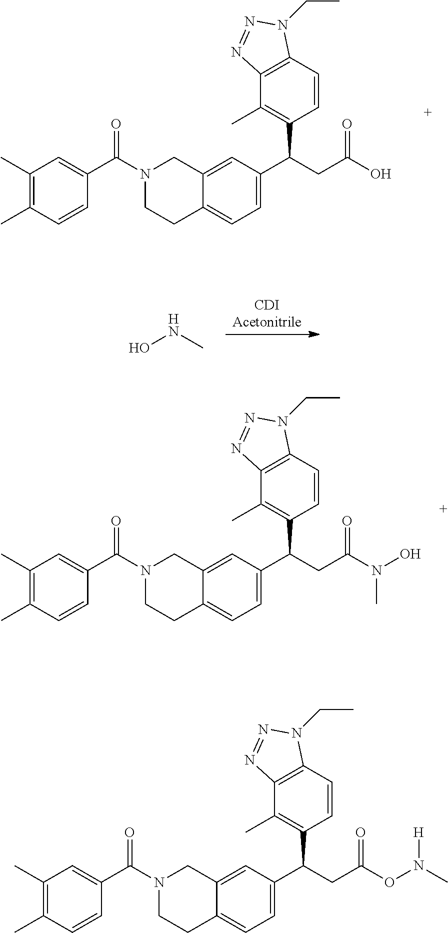

(3S)-3-[2-(3,4-dimethylbenzoyl)-3,4-dihydro-1H-isoquinolin-7-yl]-3-(1-ethyl-4-methyl-benzotriazol-5-yl)propanoic acid (100.00 mg, 201.37 umol) in Acetonitrile (2.00 mL) was added CDI (39.18 mg, 241.64 umol) and stirred at rt for 45 min, then N-Methylhydroxylamine (33.64 mg, 402.74 umol, Hydrochloride) was added and stirred overnight. The crude was purified with prep HPLC to give (3S)-3-[2-(3,4-dimethylbenzoyl)-3,4-dihydro-1H-isoquinolin-7-yl]-3-(1-ethyl-4-methyl-benzotriazol-5-yl)-N-hydroxy-N-methyl-propanamide (15.00 mg, 27.11 umol, 13.46% yield, 95% purity). LCMS: Rt=1.47 min, m/z=526.2. ¹H NMR (400 MHz, CHLOROFORM-d) δ 7.30-7.58 (m, 2H), 6.49-7.24 (m, 7H), 4.39-5.88 (m, 5H), 2.49-4.09 (m, 12H), 2.17-2.41 (m, 6H), 1.63 (br. s., 3H).

And side product methylamino (3S)-3-[2-(3,4-dimethylbenzoyl)-3,4-dihydro-1H-isoquinolin-7-yl]-3-(1-ethyl-4-methyl-benzotriazol-5-yl)propanoate (10.00 mg, 18.07 umol, 8.98% yield, 95% purity). LCMS: Rt=1.52 min, m/z=526.2. ¹H NMR (400 MHz, CHLOROFORM-d) δ 7.31-7.49 (m, 2H), 6.45-7.24 (m, 6H), 4.25-5.21 (m, 5H), 2.44-4.15 (m, 12H), 2.18-2.37 (m, 6H), 1.64 (br. s., 3H).

### Example 125

### (S)-4-(2-(3,4-dimethylbenzoyl)-1,2,3,4-tetrahydroisoquinolin-7-yl)-4-(1-ethyl-4-methyl-1H-benzo[d][1,2,3]triazol-5-yl)-1-hydroxybutan-2-one

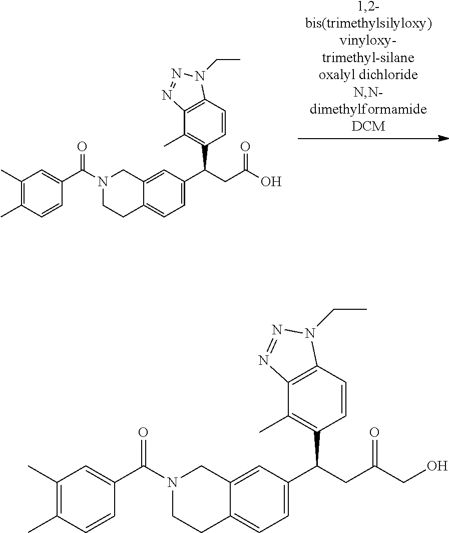

(3S)-3-[2-(3,4-dimethylbenzoyl)-3,4-dihydro-1H-isoquinolin-7-yl]-3-(1-ethyl-4-methyl-benzotriazol-5-yl)propanoic acid (85.00 mg, 171.16 umol) in DCM (2.00 mL) was added 2 drops of N,N-dimethylformamide (2.50 mg, 34.23 umol, 2.66 uL) and oxalyl dichloride (32.59 mg, 256.74 umol, 21.73 uL) and stirred at rt overnight. The reaction was concentrated to give acid chloride. 1,2-bis(trimethylsilyloxy)vinyloxy-trimethyl-silane (150.24 mg, 513.48 umol, 168.81 uL) was added and heated to 90° C. for 4 h. After cooling to rt and concentrated, dioxane (5 mL) and 1N HCl (2 mL aqueous) were added. The resulting mixture was heated at 95° C. for 30 minutes, and then quenched by pouring into saturated sodiumbicarbonate/ice-water (50 mL). The product was extracted with ethyl acetate (3×50 mL), and the extracts were dried over sodium sulfate and concentrated. Purification of the residue by silica gel chromatography eluting with diethyl etherhexanes (2:3) provided the title compound. LCMS: Rt=1.45 min, m/z=511.2. ¹H NMR (400 MHz, CHLOROFORM-d) δ 9.95 (s, 1H), 7.30-7.76 (m, 2H), 6.61-7.25 (m, 6H), 2.60-5.27 (m, 13H), 2.05-2.46 (m, 9H), 1.63 (t, J=7.03 Hz, 3H).

### Example 126

### (3S)-3-[2-(3,4-dimethylbenzoyl)-3,4-dihydro-1H-isoquinolin-7-yl]-3-(1-ethyl-4-methyl-benzotriazol-5-yl)propanehydrazide

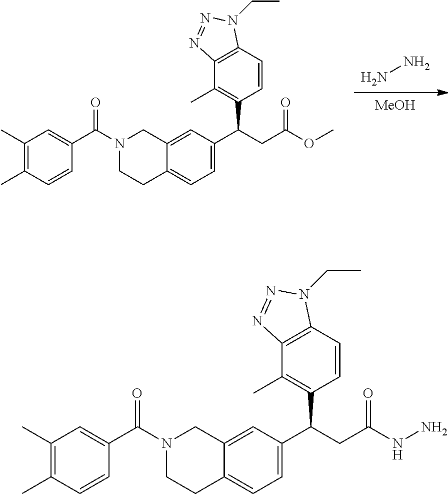

methyl (3S)-3-[2-(3,4-dimethylbenzoyl)-3,4-dihydro-1H-isoquinolin-7-yl]-3-(1-ethyl-4-methyl-benzotriazol-5-yl)propanoate (54.00 mg, 105.75 umol) in MeOH (2.00 mL) was added hydrazine (1 M, 211.50 uL) and refluxed overnight. The crude was purified with prep HPLC to give (3S)-3-[2-(3,4-dimethylbenzoyl)-3,4-dihydro-1H-isoquinolin-7-yl]-3-(1-ethyl-4-methyl-benzotriazol-5-yl)propanehydrazide (32.10 mg, 59.72 umol, 56.47% yield, 95% purity). LCMS: Rt=1.67 min, m/z=511.2. ¹H NMR (400 MHz, CHLOROFORM-d) δ 7.29-7.52 (m, 2H), 6.45-7.25 (m, 7H), 2.82 (br. s., 16H), 2.18-2.38 (m, 6H), 1.64 (br. s., 3H).

### Example 127

### methyl (3S)-3-[2-(3,4-dimethylbenzoyl)-3,4-dihydro-1H-isoquinolin-7-yl]-3-(1-ethyl-4-methyl-benzotriazol-5-yl)propanoate

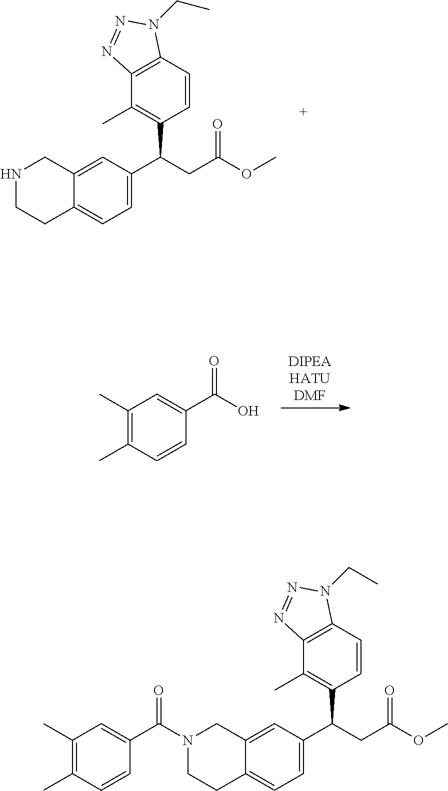

methyl (3S)-3-(1-ethyl-4-methyl-benzotriazol-5-yl)-3-(1,2,3,4-tetrahydroisoquinolin-7-yl)propanoate (999.15 mg, 2.41 mmol, Hydrochloride), 3,4-dimethylbenzoic acid (433.93 mg, 2.89 mmol), DIPEA (933.63 mg, 7.22 mmol, 1.26 mL), HATU (1.10 g, 2.89 mmol) in DMF (6.00 mL) was stirred overnight. After dilution with EtOAc and washing with water, brine, and drying over Na₂SO₄, the concentrated residue was chromatographed on Si gel (HE/EA 0-100%) to give methyl (3S)-3-[2-(3,4-dimethylbenzoyl)-3,4-dihydro-1H-isoquinolin-7-yl]-3-(1-ethyl-4-methyl-benzotriazol-5-yl)propanoate (820.00 mg, 1.61 mmol, 66.63% yield). LCMS: Rt=1.66 min, m/z=511.2.

### Example 128

### amino (3S)-3-[2-(3,4-dimethylbenzoyl)-3,4-dihydro-1H-isoquinolin-7-yl]-3-(1-ethyl-4-methyl-benzotriazol-5-yl)propanoate

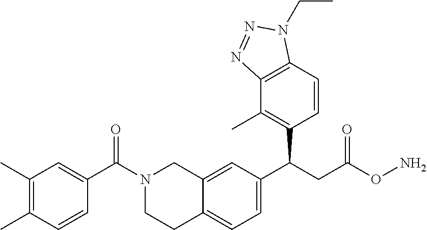 [SMILES: CCn1nnc2c(C)c([C@@H](CC(=O)ON)c3ccc4c(c3)CN(C(=O)c3ccc(C)c(C)c3)CC4)ccc21]

### 1. Preparation of (tert-butoxycarbonylamino) (3S)-3-[2-(3,4-dimethylbenzoyl)-3,4-dihydro-1H-isoquinolin-7-yl]-3-(1-ethyl-4-methyl-benzotriazol-5-yl)propanoate

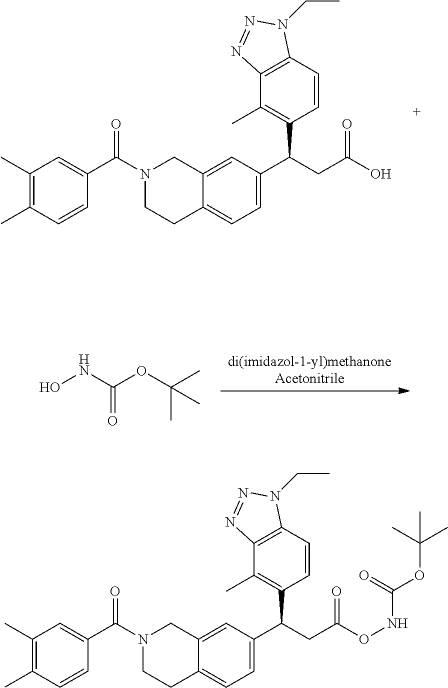

(3S)-3-[2-(3,4-dimethylbenzoyl)-3,4-dihydro-1H-isoquinolin-7-yl]-3-(1-ethyl-4-methyl-benzotriazol-5-yl)propanoic acid (181.00 mg, 364.48 umol) in Acetonitrile (2.00 mL) was added di(imidazol-1-yl)methanone (70.92 mg, 437.38 umol) and stirred at rt for 45 min, then tert-butyl N-hydroxycarbamate (97.06 mg, 728.96 umol) was added and stirred at rt for 3 h. LCMS: Rt=1.75 min, m/z=612.3. After concentration, the crude was chromatographed on Si gel (HE/EA 0-100%) to give (tert-butoxycarbonylamino) (3S)-3-[2-(3,4-dimethylbenzoyl)-3,4-dihydro-1H-isoquinolin-7-yl]-3-(1-ethyl-4-methyl-benzotriazol-5-yl)propanoate (220.00 mg, 359.64 umol, 98.67% yield).

### 2. Preparation of amino (3S)-3-[2-(3,4-dimethylbenzoyl)-3,4-dihydro-1H-isoquinolin-7-yl]-3-(1-ethyl-4-methyl-benzotriazol-5-yl)propanoate

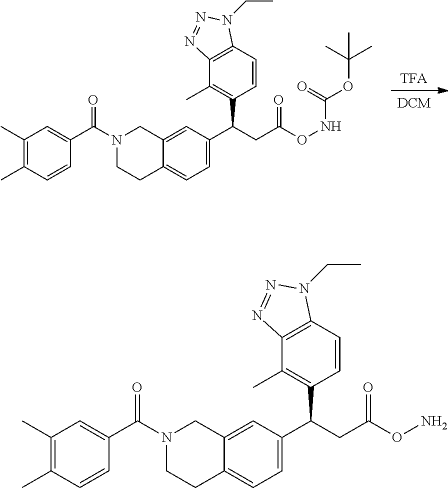

(tert-butoxycarbonylamino) (3S)-3-[2-(3,4-dimethylbenzoyl)-3,4-dihydro-1H-isoquinolin-7-yl]-3-(1-ethyl-4-methyl-benzotriazol-5-yl)propanoate (220.00 mg, 359.64 umol) in DCM (2.00 mL) was added TFA (1.49 g, 13.07 mmol, 1.00 mL) and stirred at rt for 3 h. After concentration, the crude was purified with prep HPLC to give amino (3S)-3-[2-(3,4-dimethylbenzoyl)-3,4-dihydro-1H-isoquinolin-7-yl]-3-(1-ethyl-4-methyl-benzotriazol-5-yl)propanoate (77.50 mg, 143.91 umol, 40.01% yield, 95% purity). LCMS: Rt=1.49 min, m/z=512.3. ¹H NMR (400 MHz, CHLOROFORM-d) δ 7.39 (br. s., 2H), 6.64-7.25 (m, 6H), 5.76 (br. s., 2H), 2.91-5.15 (m, 11H), 2.83 (br. s., 3H), 2.18-2.46 (m, 6H), 1.64 (br. s., 3H).

### Example 129

### (3S)-3-[2-(3,4-dimethylbenzoyl)-3,4-dihydro-1H-isoquinolin-7-yl]-3-(1-ethyl-4-methyl-benzotriazol-5-yl)propanehydroxamic acid

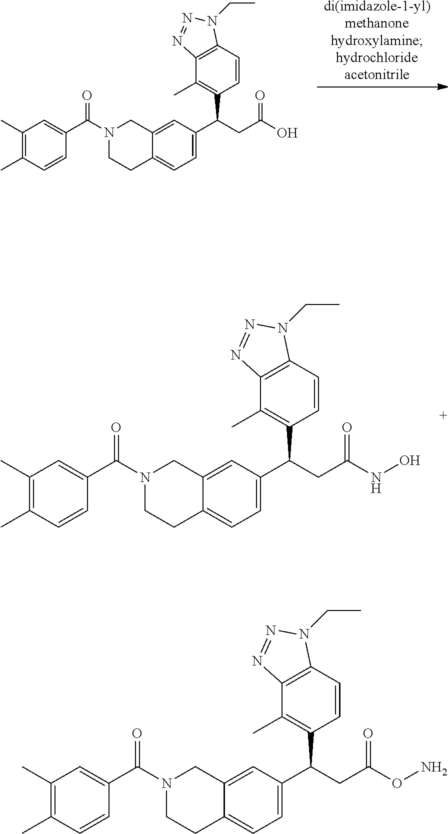 [SMILES: CCn1nnc2c(C)c([C@@H](CC(=O)O)c3ccc4c(c3)CN(C(=O)c3ccc(C)c(C)c3)CC4)ccc21 ; "di(imidazole-1-yl)methanonehydroxylamine;hydrochlorideacetonitrile" ; CCn1nnc2c(C)c([C@@H](CC(=O)NO)c3ccc4c(c3)CN(C(=O)c3ccc(C)c(C)c3)CC4)ccc21 ; CCn1nnc2c(C)c([C@@H](CC(=O)ON)c3ccc4c(c3)CN(C(=O)c3ccc(C)c(C)c3)CC4)ccc21]

(3S)-3-[2-(3,4-dimethylbenzoyl)-3,4-dihydro-1H-isoquinolin-7-yl]-3-(1-ethyl-4-methyl-benzotriazol-5-yl)propanoic acid (100.00 mg, 201.37 umol) in acetonitrile (2.00 mL) was added di(imidazol-1-yl)methanone (39.18 mg, 241.64 umol) and stirred for 45 min, then hydroxylamine;hydrochloride (27.99 mg, 402.74 umol) was added and stirred overnight. After quenched with MeOH, the crude was purified with prep HPLC to give (3S)-3-[2-(3,4-dimethylbenzoyl)-3,4-dihydro-1H-isoquinolin-7-yl]-3-(1-ethyl-4-methyl-benzotriazol-5-yl)propanehydroxamic acid (58.20 mg, 108.07 umol, 53.67% yield, 95% purity). LCMS: Rt=1.34 min, m/z=512.3. ¹H NMR (400 MHz, CHLOROFORM-d) δ 7.29-7.60 (m, 2H), 6.88-7.25 (m, 6H), 3.35-5.20 (m, 7H), 2.53-3.12 (m, 7H), 2.14-2.37 (m, 6H), 1.57 (t, J=6.65 Hz, 3H).

And peak 2 amino (3S)-3-[2-(3,4-dimethylbenzoyl)-3,4-dihydro-1H-isoquinolin-7-yl]-3-(1-ethyl-4-methyl-benzotriazol-5-yl)propanoate (8.60 mg, 15.97 umol, 7.93% yield, 95% purity). LCMS: Rt=1.50 min, m/z=512.2. ¹H NMR (400 MHz, CHLOROFORM-d) δ 7.40 (br. s., 2H), 6.86-7.25 (m, 6H), 2.90-5.32 (m, 13H), 2.82 (br. s., 3H), 2.17-2.44 (m, 6H), 1.64 (br. s., 3H).

### Example 130

### (3S)-3-[2-(3,4-dimethylbenzoyl)-3,4-dihydro-1H-isoquinolin-7-yl]-3-(1-ethyl-4-methyl-benzotriazol-5-yl)-N-methoxy-N-methyl-propanamide

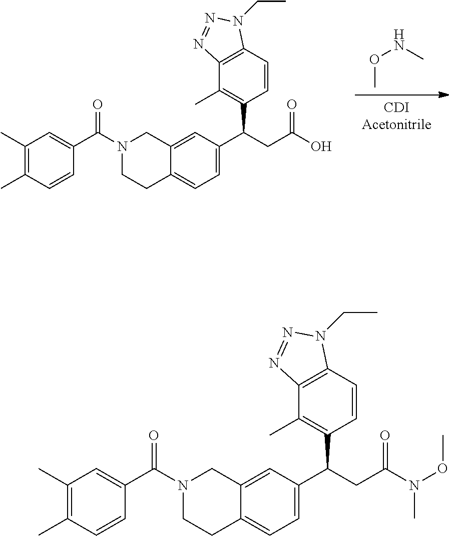

(3S)-3-[2-(3,4-dimethylbenzoyl)-3,4-dihydro-1H-isoquinolin-7-yl]-3-(1-ethyl-4-methyl-benzotriazol-5-yl)propanoic acid (100.00 mg, 201.37 umol) in Acetonitrile (2.00 mL) was added CDI (39.18 mg, 241.64 umol) and stirred at rt for 45 min, then N-methoxymethanamine (24.60 mg, 252.20 umol, Hydrochloride) was added and stirred for 3 h. The crude was purified with prep HPLC to give (3S)-3-[2-(3,4-dimethylbenzoyl)-3,4-dihydro-1H-isoquinolin-7-yl]-3-(1-ethyl-4-methyl-benzotriazol-5-yl)-N-methoxy-N-methyl-propanamide (74.00 mg, 130.26 umol, 64.69% yield, 95% purity). LCMS: Rt=1.58 min, m/z=540.3. ¹H NMR (400 MHz, CHLOROFORM-d) δ 7.30-7.51 (m, 2H), 6.56-7.24 (m, 6H), 4.30-5.43 (m, 5H), 2.51-4.16 (m, 15H), 2.07-2.43 (m, 6H), 1.63 (br. s., 3H).

### Example 131

### (3S)-3-[2-(3,4-dimethylbenzoyl)-3,4-dihydro-1H-isoquinolin-7-yl]-3-(1-ethyl-4-methyl-benzotriazol-5-yl)-N-methoxy-propanamide

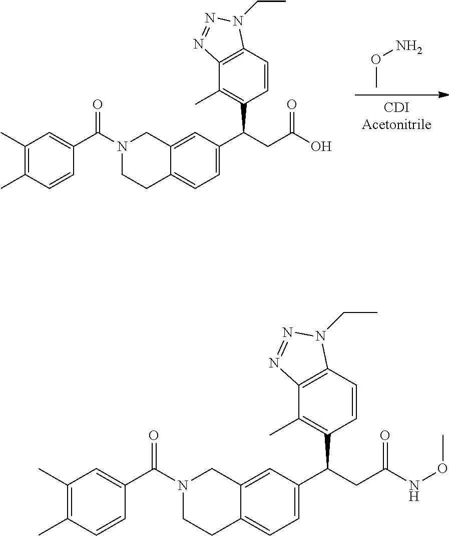

(3S)-3-[2-(3,4-dimethylbenzoyl)-3,4-dihydro-1H-isoquinolin-7-yl]-3-(1-ethyl-4-methyl-benzotriazol-5-yl)propanoic acid (100.00 mg, 201.37 umol) in Acetonitrile (2.00 mL) was added CDI (39.18 mg, 241.64 umol) and stirred at rt for 45 min, then O-methylhydroxylamine (18.95 mg, 226.89 umol, Hydrochloride) was added and stirred for 3 h. The crude was purified with prep HPLC to give (3S)-3-[2-(3,4-dimethylbenzoyl)-3,4-dihydro-1H-isoquinolin-7-yl]-3-(1-ethyl-4-methyl-benzotriazol-5-yl)-N-methoxy-propanamide (44.50 mg, 80.43 umol, 39.94% yield, 95% purity). LCMS: Rt=1.40 min, m/z 526.2. ¹H NMR (400 MHz, CHLOROFORM-d) δ 8.50 (brs, 1H), 7.33-7.60 (m, 2H), 6.59-7.25 (m, 6H), 4.35-5.29 (m, 5H), 2.46-4.17 (m, 12H), 2.17-2.39 (m, 6H), 1.64 (br. s., 3H).

### Example 132

### (3S)-3-[2-(3,4-dimethylbenzoyl)-3,4-dihydro-1H-isoquinolin-7-yl]-3-(1-ethyl-4-methyl-benzotriazol-5-yl)propanamide

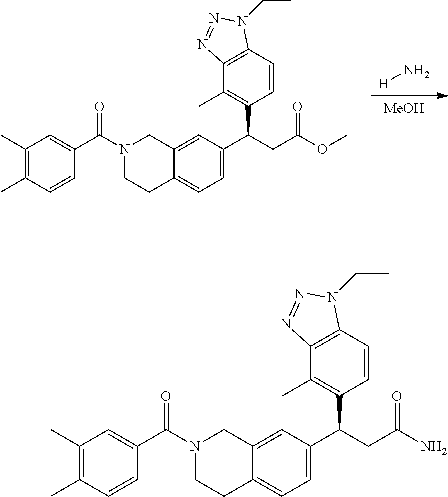

methyl (3S)-3-[2-(3,4-dimethylbenzoyl)-3,4-dihydro-1H-isoquinolin-7-yl]-3-(1-ethyl-4-methyl-benzotriazol-5-yl)propanoate (146.00 mg, 285.92 umol) in MeOH (2.00 mL) was added ammonia (7 M, 122.54 uL) and microwaved at 60° C. for 30 min. And stirred over the weekend (4 days). The crude was purified with prep HPLC to give (3S)-3-[2-(3,4-dimethylbenzoyl)-3,4-dihydro-1H-isoquinolin-7-yl]-3-(1-ethyl-4-methyl-benzotriazol-5-yl)propanamide (1.50 mg, 2.88 umol, 1.01% yield, 95% purity). LCMS: Rt=1.36 min, m/z=496.2. ¹H NMR (400 MHz, CHLOROFORM-d) δ 8.11 (s, 1H), 7.35 (br. s., 2H), 6.69-7.23 (m, 6H), 2.91-5.47 (m, 12H), 2.83 (br. s., 3H), 2.29 (d, J=8.53 Hz, 6H), 1.62 (t, J=7.28 Hz, 3H).

### Example 133

### amino (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate

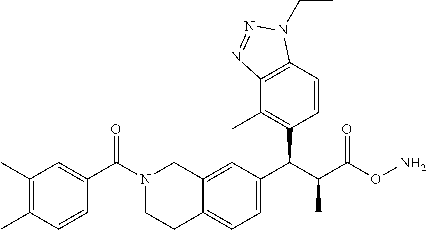 [SMILES: CCn1nnc2c(C)c([C@H](c3ccc4c(c3)CN(C(=O)c3ccc(C)c(C)c3)CC4)[C@H](C)C(=O)ON)ccc21]

### 1. Preparation of (tert-butoxycarbonylamino) (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate

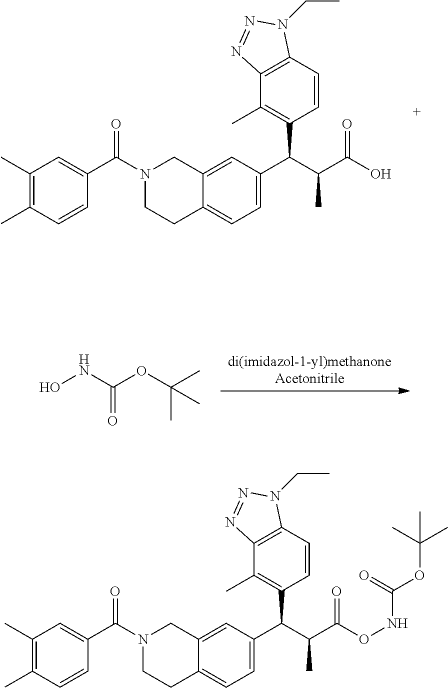

(2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoic acid (52.80 mg, 103.40 umol) in Acetonitrile (2.00 mL) was added di(imidazol-1-yl)methanone (20.12 mg, 124.08 umol) and stirred at rt for 45 min, tert-butyl N-hydroxycarbamate (27.54 mg, 206.80 umol) was added and stirred for 3 h. After concentration, the crude was chromatographed on si gel (HE/EA 0-100%) to give (tert-butoxycarbonylamino) (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate (28.50 mg, 45.54 umol, 44.05% yield). LCMS: Rt=1.82 min, m/z=626.3.

### 2. Preparation of amino (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate

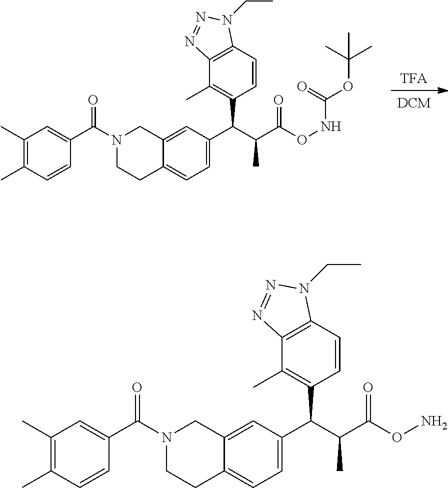

(tert-butoxycarbonylamino) (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate (28.50 mg, 45.54 umol) in DCM (2.00 mL) was added TFA (519.25 mg, 4.55 mmol, 348.49 uL) and stirred at rt for 3 h. The crude was purified with prep HPLC to give amino (2S,3S)-3-[2-(3,4-dimethylbenzoyl)-3,4-dihydro-1H-isoquinolin-7-yl]-3-(1-ethyl-4-methyl-benzotriazol-5-yl)-2-methyl-propanoate (4.00 mg, 7.23 umol, 15.87% yield, 95% purity). LCMS: Rt=1.53 min, m/z=526.2. ¹H NMR (400 MHz, CHLOROFORM-d) δ 7.39 (br. s., 2H), 6.81-7.24 (m, 6H), 2.88-5.15 (m, 10H), 2.83 (br. s., 3H), 2.15-2.45 (m, 6H), 1.49-1.73 (m, 3H), 1.26 (br. s., 3H).

### Examples 134 and 135

### (3R)-3-[7-(3,4-dimethylbenzoyl)-6,8-dihydro-5H-1,7-naphthyridin-2-yl]-3-(1-ethyl-4-methyl-benzotriazol-5-yl)propanoic acid and (3S)-3-[7-(3,4-dimethylbenzoyl)-6,8-dihydro-5H-1,7-naphthyridin-2-yl]-3-(1-ethyl-4-methyl-benzotriazol-5-yl)propanoic acid

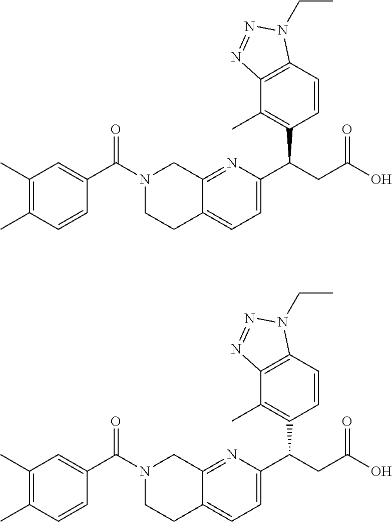 [SMILES: CCn1nnc2c(C)c([C@@H](CC(=O)O)c3ccc4c(n3)CN(C(=O)c3ccc(C)c(C)c3)CC4)ccc21 ; CCn1nnc2c(C)c([C@H](CC(=O)O)c3ccc4c(n3)CN(C(=O)c3ccc(C)c(C)c3)CC4)ccc21]

### 1. Synthesis of 1-ethyl-4-methyl-5-(4,4,5,5-tetramethyl-1,3,2-dioxaborolan-2-yl)-1H-benzo[d][1,2,3]triazole

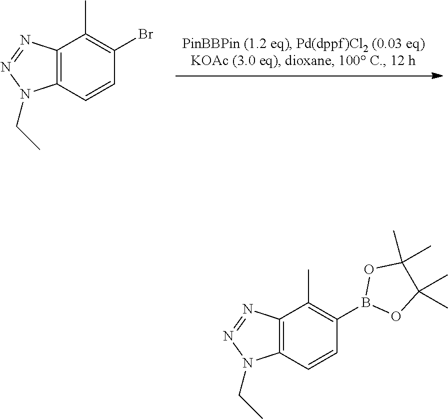 [SMILES: CCn1nnc2c(C)c(Br)ccc21 ; "PinBBPin (1.2 eq), Pd(dppf)Cl2 (0.03 eq)KOAc (3.0 eq), dioxane, 100° C., 12 h" ; CCn1nnc2c(C)c(B3OC(C)(C)C(C)(C)O3)ccc21]

To a mixture of 5-bromo-1-ethyl-4-methyl-1H-benzo[d][1,2,3]triazole (60 g, 251 mmol) and PinB-BPin (76.5 g, 301.2 mmol) in dioxane (500 mL), KOAc (73.8 g, 753 mmol) was added. Then Pd(dppf)Cl₂ (5.5 g, 7.53 mmol) was added quickly under N₂ atmosphere. The mixture was stirred at 100° C. for 12 h under N₂ atmosphere. After cooled down, the salts were filtered out, the resulting filtrate was concentrated and purified by silica gel column (PE) to give 1-ethyl-4-methyl-5-(4,4,5,5-tetramethyl-1,3,2-dioxaborolan-2-yl)-1H-benzo[d][1,2,3]triazole (58.5 g, yield: 81%) as a brown solid. ESI-MS (M+H)⁺: 288.1. ¹H NMR (400 MHz, CDCl₃) δ: 7.85 (d, J=8.4 Hz, 1H), 7.31-7.264 (m, 1H), 4.69-4.64 (m, 2H), 3.04 (s, 3H), 1.62-1.58 (m, 3H), 1.38 (s, 12H).

### 2. Synthesis of methyl (E)-3-(4,4,5,5-tetramethyl-1,3,2-dioxaborolan-2-yl)acrylate

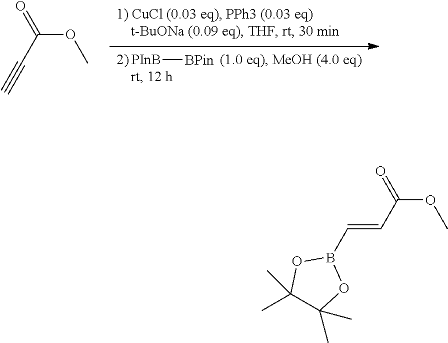

A mixture of CuCl (1.5 g, 15 mmol), PPh₃ (3.9 g, 15 mmol) and t-BuONa (4.3 g, 45 mmol) in THF (600 mL) was stirred at rt under N₂ atmosphere for 30 min before PinB-BPin (127 g, 500 mmol) was added. The mixture was stirred for 10 min and then methyl propiolate (42 g, 500 mmol) and MeOH (50 mL, 1.2 mol) were added. The mixture was stirred at rt for 12 h under N₂ atmosphere. After concentration, the residue was purified by silica gel column (PE) to give methyl (E)-3-(4,4,5,5-tetramethyl-1,3,2-dioxaborolan-2-yl)acrylate (42 g, yield: 40%) as a colorless oil. ESI-MS (M+H)⁺: 131.1. ¹H NMR (400 MHz, CDCl₃) δ: 6.80-6.75 (m, 1H), 6.65-6.60 (m, 1H), 3.76 (s, 3H), 1.28 (s, 12H).

### 3. Synthesis of ethyl (E)-3-(3-aminopyridin-4-yl)acrylate

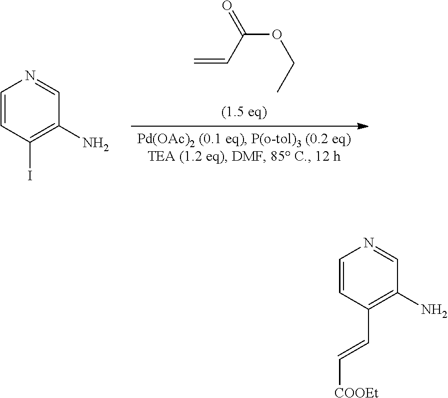

To a mixture of 4-iodopyridin-3-amine (40 g, 182 mmol) and ethyl acrylate (27 g, 272 mmol) in DMF (300 mL), DIPEA (28 g, 218 mmol) was added. Then Pd(OAc)₂ (4 g, 18 mmol) and P(o-tol)₃ (11 g, 36 mmol) were added quickly under N₂ atmosphere. The mixture was stirred at 85° C. for 12 h under N₂ atmosphere. After cooling down, the solvent was removed under reduced pressure. The residue was purified by silica gel column (DCM/MeOH=20/1) to give ethyl (E)-3-(3-aminopyridin-4-yl)acrylate (20 g, yield: 57%) as a yellow oil. ESI-MS (M+H)⁺: 193.1. ¹H NMR (400 MHz, CDCl₃) δ: 8.15 (s, 1H), 8.02-7.98 (m, 1H), 7.72 (d, J=16.4 Hz, 1H), 7.18 (d, J=5.2 Hz, 1H), 6.48 (d, J=16 Hz, 1H), 4.27 (q, J=7.2 Hz, 2H), 4.07 (s, 2H), 1.34 (t, J=7.2Hz, 3H).

### 4. Synthesis of 1,7-naphthyridin-2(1H)-one

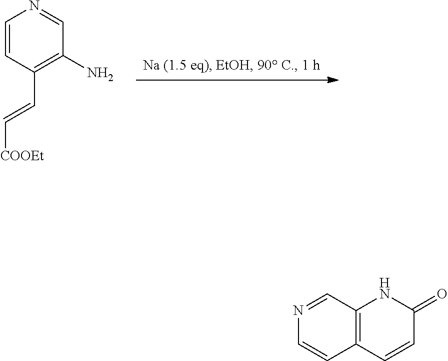 [SMILES: CCOC(=O)/C=C/c1ccncc1N ; "Na (1.5 eq), EtOH, 90° C., 1 h" ; O=c1ccc2ccncc2[nH]1]

Na (3.6 g, 156 mmol) was added to EtOH (300 mL) by portion carefully, and ethyl (E)-3-(3-aminopyridin-4-yl)acrylate (20 g, 104 mmol) was added after Na was consumed completely. The mixture was stirred at 90° C. for 1 h. After cooling down, the mixture was concentrated and purified by silica gel column (DCM/MeOH=10/1) to give 1,7-naphthyridin-2(1H)-one (10 g, yield: 66%) as a yellow solid. ESI-MS (M+H)⁺: 147.1. ¹H NMR (400 MHz, CDCl₃) δ: 8.76 (s, 1H), 8.46 (d, J=5.2 Hz, 1H), 7.78 (d, J=9.6 Hz, 1H), 7.44 (d, J=5.2 Hz, 1H), 6.90 (d, J=9.6 Hz, 1H).

### 5. Synthesis of 7-benzyl-2-oxo-1,2-dihydro-1,7-naphthyridin-7-ium Bromide

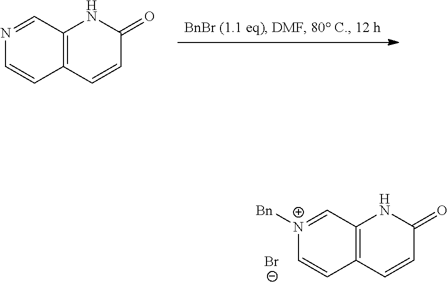 [SMILES: O=c1ccc2ccncc2[nH]1 ; "BnBr (1.1 eq), DMF, 80° C., 12 h" ; [Br-] ; O=c1ccc2cc[n+](Cc3ccccc3)cc2[nH]1]

To a mixture of 1,7-naphthyridin-2(1H)-one (10 g, 68 mmol) in DMF (100 mL), BnBr (12.8 g, 75 mmol) was added. The mixture was stirred at 80° C. for 12 h. After cooling down, the reaction mixture was diluted with DCM/PE (100 mL/200 mL). The precipitate was filtered and dried to give 7-benzyl-2-oxo-1,2-dihydro-1,7-naphthyridin-7-ium Bromide (13.1 g, yield: 61%) as a yellow solid. ESI-MS (M+H)⁺: 237.1.

### 6. Synthesis of 7-benzyl-5,6,7,8-tetrahydro-1,7-naphthyridin-2(1H)-one

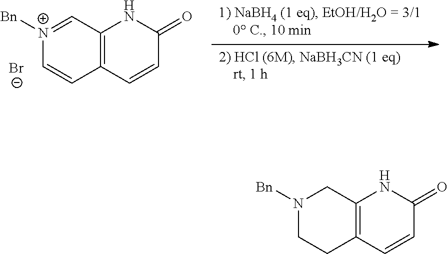

To a mixture of 7-benzyl-2-oxo-1,2-dihydro-1,7-naphthyridin-7-ium Bromide (20 g, 63.3 mmol) in EtOH/H₂O (300 mL/100 mL) was added NaBH₄ (2.4 g, 63.3 mmol) carefully at 0° C. The mixture was stirred at 0° C. for 10 min before HCl (40 mL, 6M) was added. Then NaBH₃CN (4 g, 63.3 mmol) was added. The mixture was stirred at rt for 1 h. The reaction mixture was basified with 2N NaOH to pH=10 and extracted with DCM (300 mL×3). The organic layer was washed with brine (200 mL×3), dried over sodium sulfate and concentrated under reduced pressure. The residue was purified by silica gel column (DCM/MeOH=50/1 to 20/1) to give 7-benzyl-5,6,7,8-tetrahydro-1,7-naphthyridin-2(1H)-one (10 g, yield: 66%) as a yellow solid. ESI-MS (M+H)⁺: 241.1. ¹H NMR (400 MHz, CDCl₃) δ: 13.14 (s, 1H), 7.37-7.21 (m, 6H), 6.40 (d, J=9.2 Hz, 1H), 3.69 (s, 2H), 3.63 (s, 2H), 2.65-2.62 (m, 2H), 2.56-2.53 (m, 2H).

### 7. Synthesis of 7-benzyl-5,6,7,8-tetrahydro-1,7-naphthyridin-2-yl trifluoromethanesulfonate

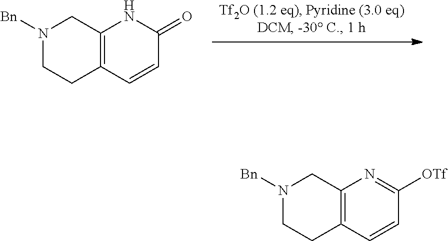 [SMILES: O=c1ccc2c([nH]1)CN(Cc1ccccc1)CC2 ; "Tf2O (1.2 eq), Pyridine (3.0 eq)DCM, -30° C., 1 h" ; O=S(=O)(Oc1ccc2c(n1)CN(Cc1ccccc1)CC2)C(F)(F)F]

To a mixture of 7-benzyl-5,6,7,8-tetrahydro-1,7-naphthyridin-2(1H)-one (60 g, 250 mmol) in DCM (600 mL) was added pyridine (59.2 g, 750 mmol) and Tf₂O (84.6 g, 300 mmol) at −30° C. The mixture was stirred at −30° C. for 1 h. After the reaction was completed, the reaction was diluted with DCM (200 mL), washed with water (200 mL), brine (200 mL), dried and concentrated under reduced pressure. The residue was purified by silica gel column (PE/EA=5/1) to give 7-benzyl-5,6,7,8-tetrahydro-1,7-naphthyridin-2-yl trifluoromethanesulfonate (91.4 g, yield: 98%) as yellow oil. ESI-MS (M+H)⁺: 373.1. ¹H NMR (400 MHz, CDCl₃) δ: 7.58 (d, J=8.0 Hz, 1H), 7.38-7.28 (m, 5H), 6.95 (d, J=8.0 Hz, 1H), 3.73 (s, 2H), 3.70 (s, 2H), 2.91-2.89 (m, 2H), 2.78-2.75 (m, 2H).

### 8. Synthesis of methyl (E)-3-(7-benzyl-5,6,7,8-tetrahydro-1,7-naphthyridin-2-yl)acrylate

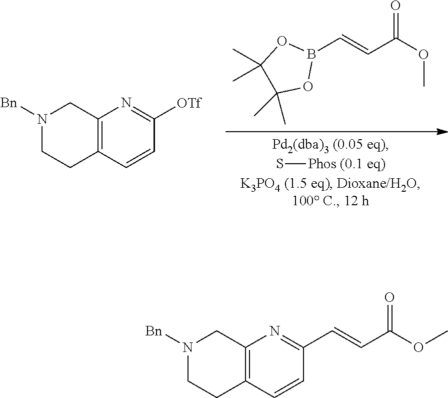

To a mixture of 7-benzyl-5,6,7,8-tetrahydro-1,7-naphthyridin-2-yl trifluoromethanesulfonate (50 g, 134.4 mmol) and methyl (E)-3-(4,4,5,5-tetramethyl-1,3,2-dioxaborolan-2-yl)acrylate (71.2 g, 336 mmol) in dioxane/H₂O (500 mL/100 mL) was added K₃PO₄ (42.7 g, 201.6 mmol). Then Pd₂(dba)₃ (6.1 g, 6.72 mmol) and S-Phos (5.5 g, 13.44 mmol) were added quickly under N₂ atmosphere. The mixture was stirred at 100° C. for 12 h under N₂ atmosphere. After cooled down, the mixture was concentrated and purified by silica gel column (PE/EA=8/1) to give methyl (E)-3-(7-benzyl-5,6,7,8-tetrahydro-1,7-naphthyridin-2-yl)acrylate (43.5 g, yield: 100%) as a yellow oil. ESI-MS (M+H)⁺: 309.1. ¹H NMR (400 MHz, CDCl₃) δ: 7.63 (d, J=16 Hz, 1H), 7.43-7.27 (m, 7H), 6.81 (d, J=16 Hz, 1H), 3.79 (s, 3H), 3.74 (s, 4H), 2.92-2.89 (m, 2H), 2.78-2.75 (m, 2H).

### 9. Synthesis of methyl (E)-3-(5,6,7,8-tetrahydro-1,7-naphthyridin-2-yl)acrylate

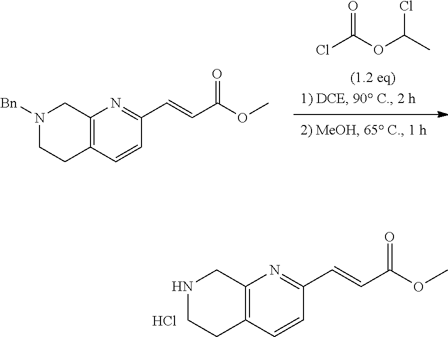

To a solution of methyl (E)-3-(7-benzyl-5,6,7,8-tetrahydro-1,7-naphthyridin-2-yl)acrylate (60.6 g, 196.7 mmol) in DCE (600 mL) was added 1-chloroethyl carbonochloridate (33.5 g, 236.1 mmol). The solution was stirred at 90° C. for 2 h. After cooled down, the solvent was removed under reduced pressure. The residue was dissolved in MeOH (500 mL) and stirred at 65° C. for 1 h. After concentration, the residue was washed with Et₂O (200 mL×2). The solid was concentrated to give methyl (E)-3-(5,6,7,8-tetrahydro-1,7-naphthyridin-2-yl)acrylate hydrochloride (27.6 g, yield: 55%) as a white solid. ESI-MS (M+H)⁺: 219.1. ¹H NMR (400 MHz, DMSO-d₆) δ: 9.71 (s, 2H), 7.77-7.66 (m, 3H), 6.88 (d, J=15.6 Hz, 1H), 4.33-4.26 (m, 2H), 3.75 (s, 3H), 3.45-3.36 (m, 2H), 2.51-2.49 (m, 2H).

### 10. Synthesis of tert-butyl (E)-2-(3-methoxy-3-oxoprop-1-en-1-yl)-5,8-dihydro-1,7-naphthyridine-7(6H)-carboxylate

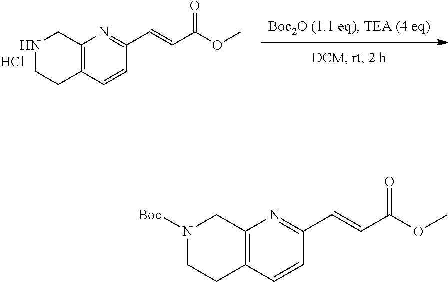 [SMILES: Cl ; COC(=O)/C=C/c1ccc2c(n1)CNCC2 ; COC(=O)/C=C/c1ccc2c(n1)CN(C(=O)OC(C)(C)C)CC2]

To a mixture of methyl (E)-3-(5,6,7,8-tetrahydro-1,7-naphthyridin-2-yl)acrylate hydrochloride (45.3 g, 178.3 mmol) in DCM (500 mL) was added TEA (72 g, 713.2 mmol) and Boc₂O (42.7 g, 196.1 mmol). The mixture was stirred at rt for 2 h. After diluted with water (500 mL), the mixture was extracted with DCM (500 mL×2). The organic layers were combined and concentrated under reduced pressure. The residue was purified by silica gel column (PE/EA=5:1) to give tert-butyl (E)-2-(3-methoxy-3-oxoprop-1-en-1-yl)-5,8-dihydro-1,7-naphthyridine-7(6H)-carboxylate (50.1 g, yield: 88%) as a white solid. ESI-MS (M+H)⁺: 319.1. ¹H NMR (400 MHz, CDCl₃) δ: 7.66 (d, J=16 Hz, 1H), 7.46 (d, J=7.6 Hz, 1H), 7.26 (s, 1H), 6.88 (d, J=16 Hz, 1H), 4.67 (s, 2H), 3.81 (s, 3H), 3.71-3.68 (m, 2H), 2.88-2.85 (m, 2H), 1.50 (s, 9H).

### 11. Synthesis of tert-butyl 2-(1-(1-ethyl-4-methyl-1H-benzo[d][1,2,3]triazol-5-yl)-3-methoxy-3-oxopropyl)-5,8-dihydro-1,7-naphthyridine-7(6H)-carboxylate

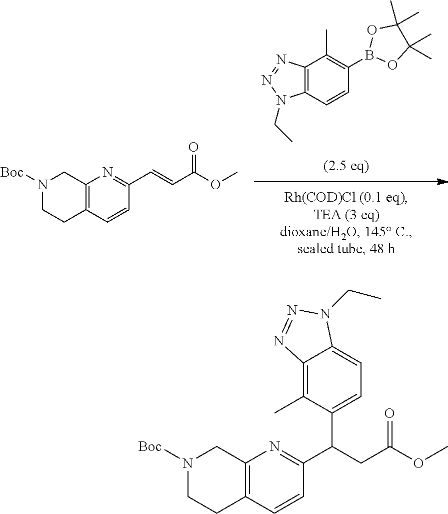 [SMILES: CCn1nnc2c(C)c(B3OC(C)(C)C(C)(C)O3)ccc21 ; COC(=O)/C=C/c1ccc2c(n1)CN(C(=O)OC(C)(C)C)CC2 ; CCn1nnc2c(C)c(C(CC(=O)OC)c3ccc4c(n3)CN(C(=O)OC(C)(C)C)CC4)ccc21]

A mixture of tert-butyl (E)-2-(3-methoxy-3-oxoprop-1-en-1-yl)-5,8-dihydro-1,7-naphthyridine-7(6H)-carboxylate (4 g, 12.6 mmol), 1-ethyl-4-methyl-5-(4,4,5,5-tetramethyl-1,3,2-dioxaborolan-2-yl)-1H-benzo[d][1,2,3]triazole (9 g, 31.5 mmol) and TEA (3.8 g, 37.8 mmol) in dioxane/H₂O (100 mL/20 mL) was degassed for 10 min before [Rh(COD)Cl]₂ (620 mg, 1.2 mmol) was added, and then the mixture was degassed for another 10 min. The mixture was stirred at 145° C. for 48 h in a sealed tube. After cooled down, the solvent was removed under reduced pressure. The residue was diluted with water (100 mL) and extracted with DCM (100 mL×3). The combined organic layers were concentrated and purified by silica gel column (PE/EA=10/1 to 2/1) to give tert-butyl 2-(1-(1-ethyl-4-methyl-1H-benzo[d][1,2,3]triazol-5-yl)-3-methoxy-3-oxopropyl)-5,8-dihydro-1,7-naphthyridine-7(6H)-carboxylate (2.1 g, yield: 35%) as a yellow solid. ESI-MS (M+H)⁺: 480.2. ¹H NMR (400 MHz, CDCl₃) δ: 7.38 (d, J=8.4 Hz, 1H), 7.28-7.24 (m, 2H), 6.82 (d, J=8.0 Hz, 1H), 5.09-5.06 (m, 1H), 4.65-4.60 (m, 4H), 3.67 (s, 2H), 3.61 (s, 3H), 3.54-3.48 (m, 1H), 2.93-2.91 (m, 4H), 2.76-7.74 (m, 2H), 1.60-1.57 (m, 3H), 1.51 (9H).

### 12. Synthesis of methyl 3-(1-ethyl-4-methyl-1H-benzo[d][1,2,3]triazol-5-yl)-3-(5,6,7,8-tetrahydro-1,7-naphthyridin-2-yl)propanoate hydrogen chloride

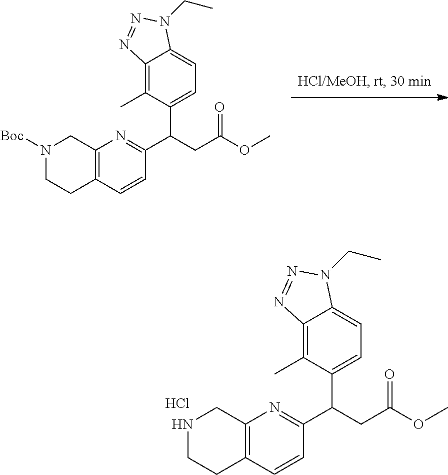 [SMILES: CCn1nnc2c(C)c(C(CC(=O)OC)c3ccc4c(n3)CN(C(=O)OC(C)(C)C)CC4)ccc21 ; Cl ; CCn1nnc2c(C)c(C(CC(=O)OC)c3ccc4c(n3)CNCC4)ccc21]

A solution of tert-butyl 2-(1-(1-ethyl-4-methyl-1H-benzo[d][1,2,3]triazol-5-yl)-3-methoxy-3-oxopropyl)-5,8-dihydro-1,7-naphthyridine-7(6H)-carboxylate (4.4 g, 9.2 mmol) in HCl/MeOH (40 mL, 2M) was stirred at rt for 30 min. The solvent was removed and the residue was concentrated to give methyl 3-(1-ethyl-4-methyl-1H-benzo[d][1,2,3]triazol-5-yl)-3-(5,6,7,8-tetrahydro-1,7-naphthyridin-2-yl)propanoate hydrogen chloride (4.8 g, yield: 100%) as a yellow solid. ESI-MS (M+H)⁺: 380.2.

### 13. Separation of methyl (S)-3-(1-ethyl-4-methyl-1H-benzo[d][1,2,3]triazol-5-yl)-3-(5,6,7,8-tetrahydro-1,7-naphthyridin-2-yl)propanoate and methyl (R)-3-(1-ethyl-4-methyl-1H-benzo[d][1,2,3]triazol-5-yl)-3-(5,6,7,8-tetrahydro-1,7-naphthyridin-2-yl)propanoate

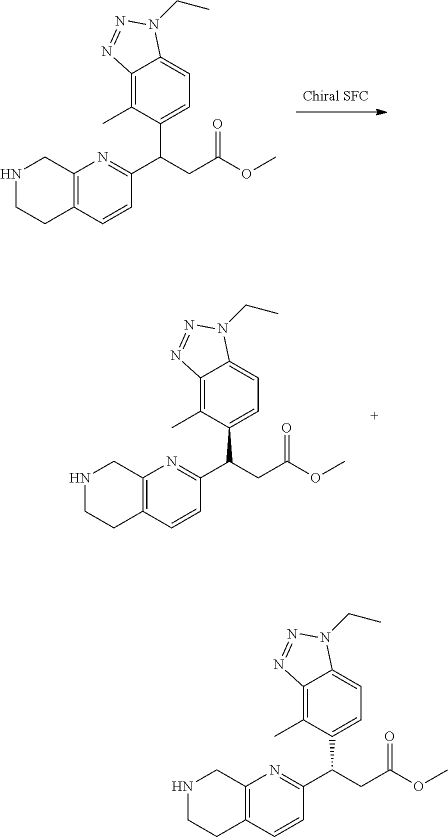

The recamic methyl 3-(1-ethyl-4-methyl-1H-benzo[d][1,2,3]triazol-5-yl)-3-(5,6,7,8-tetrahydro-1,7-naphthyridin-2-yl)propanoate was separated under the following SFC Conditions (Column: AY-H 100*4.6 mm, 5 um (Daicel); Column temperature: 40° C.; Mobile phase: CO₂/MeOH (0.2% Methanol Ammonia)=70/30; Flow rate: 80 g/min; Back pressure: 122 bar; Detection wavelength: 214 nm; Cycle time: 10.5 min) to give peak 1 as (S)-3-(1-ethyl-4-methyl-1H-benzo[d][1,2,3]triazol-5-yl)-3-(5,6,7,8-tetrahydro-1,7-naphthyridin-2-yl)propanoate. ¹H NMR (400 MHz, CD₃OD) δ: 8.01 (d, J=8.4 Hz, 1H), 7.74 (d, J=8.8 Hz, 1H), 7.64-7.60 (m, 2H), 5.31-5.27 (m, 1H), 4.84-4.78 (m, 2H), 4.67-4.57 (m, 2H), 3.62-3.52 (m, 6H), 3.26-3.20 (m, 3H), 2.90 (s, 3H), 1.63 (t, J=7.2 Hz, 3H). And Peak 2 as methyl (R)-3-(1-ethyl-4-methyl-1H-benzo[d][1,2,3]triazol-5-yl)-3-(5,6,7,8-tetrahydro-1,7-naphthyridin-2-yl)propanoate. ¹H NMR (400 MHz, CD₃OD) δ: 7.94-7.88 (m, 1H), 7.72-7.48 (m, 3H), 5.28-5.25 (m, 1H), 4.82-4.77 (m, 2H), 4.61-4.54 (m, 2H), 3.62-3.50 (m, 6H), 3.17-3.22 (m, 3H), 3.90 (s, 3H), 1.62 (t, J=7.2 Hz, 3H).

### 14. Preparation of (3S)-3-[7-(3,4-dimethylbenzoyl)-6,8-dihydro-5H-1,7-naphthyridin-2-yl]-3-(1-ethyl-4-methyl-benzotriazol-5-yl)propanoate

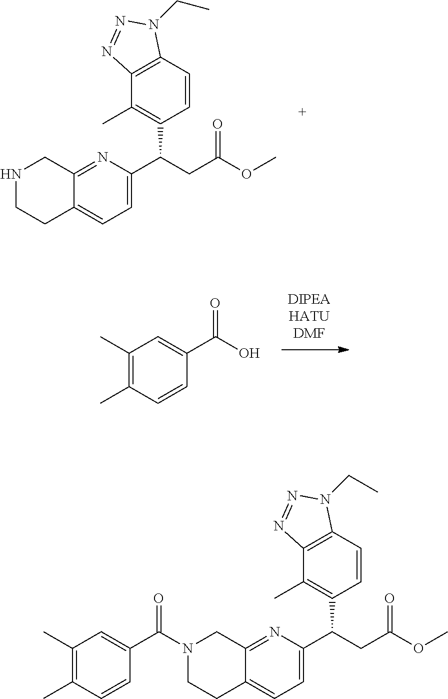

methyl (3S)-3-(1-ethyl-4-methyl-benzotriazol-5-yl)-3-(5,6,7,8-tetrahydro-1,7-naphthyridin-2-yl)propanoate (100.00 mg, 240.43 umol, Hydrochloride), 3,4-dimethylbenzoic acid (43.33 mg, 288.52 umol), DIPEA (93.22 mg, 721.29 umol, 125.97 uL) and HATU (109.99 mg, 288.52 umol) in DMF (2.00 mL) was stirred at rt for overnight. The crude was purified with prep HPLC to give methyl (3S)-3-[7-(3,4-dimethylbenzoyl)-6,8-dihydro-5H-1,7-naphthyridin-2-yl]-3-(1-ethyl-4-methyl-benzotriazol-5-yl)propanoate (114.00 mg, 182.51 umol, 75.91% yield, Trifluoroacetate). LCMS: RT=1.47 min, m/z=512.2.

### 15. Preparation of methyl (3R)-3-[7-(3,4-dimethylbenzoyl)-6,8-dihydro-5H-1,7-naphthyridin-2-yl]-3-(1-ethyl-4-methyl-benzotriazol-5-yl)propanoate

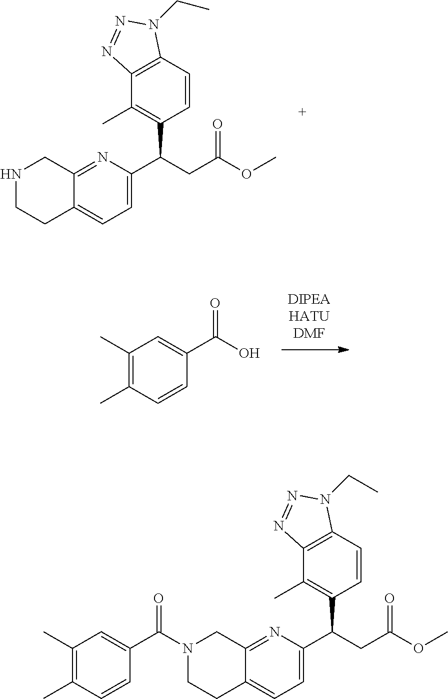

methyl (3R)-3-(1-ethyl-4-methyl-benzotriazol-5-yl)-3-(5,6,7,8-tetrahydro-1,7-naphthyridin-2-yl)propanoate (100.00 mg, 240.43 umol, Hydrochloride), 3,4-dimethylbenzoic acid (43.33 mg, 288.52 umol), DIPEA (93.22 mg, 721.29 umol, 125.97 uL) and HATU (109.99 mg, 288.52 umol) in DMF (2.00 mL) was stirred at rt for overnight. The crude was purified with prep HPLC to give methyl (3R)-3-[7-(3,4-dimethylbenzoyl)-6,8-dihydro-5H-1,7-naphthyridin-2-yl]-3-(1-ethyl-4-methyl-benzotriazol-5-yl)propanoate (150.00 mg, 240.14 umol, 99.88% yield, Trifluoroacetate). LCMS: Rt=1.46 min, m/z 512.0.

### 16. Preparation of (3R)-3-[7-(3,4-dimethylbenzoyl)-6,8-dihydro-5H-1,7-naphthyridin-2-yl]-3-(1-ethyl-4-methyl-benzotriazol-5-yl)propanoic acid

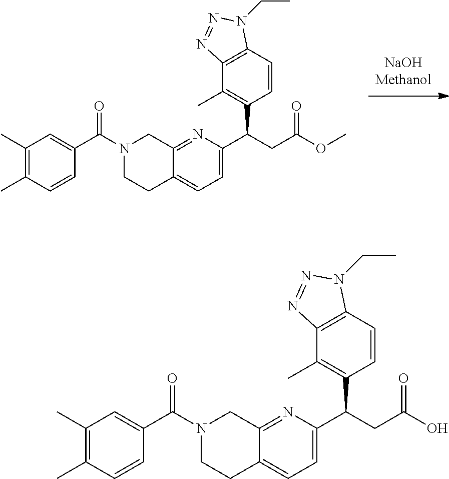

methyl (3R)-3-[7-(3,4-dimethylbenzoyl)-6,8-dihydro-5H-1,7-naphthyridin-2-yl]-3-(1-ethyl-4-methyl-benzotriazol-5-yl)propanoate (150.00 mg, 240.14 umol, Trifluoroacetate) in Methanol (2.00 mL) was added NaOH (2 M, 360.21 uL) and microwaved at 100° C. for 30 min. After neutralized with 2M HCl, the crude was purified with prep HPLC to give (3R)-3-[7-(3,4-dimethylbenzoyl)-6,8-dihydro-5H-1,7-naphthyridin-2-yl]-3-(1-ethyl-4-methyl-benzotriazol-5-yl)propanoic acid (80.40 mg, 125.09 umol, 52.09% yield, 95% purity, Trifluoroacetate). LCMS: Rt=1.27 min, m/z=498.2. ¹H NMR (400 MHz, CHLOROFORM-d) δ 9.30 (br. s., 2H), 7.61 (d, J=8.03 Hz, 1H), 7.34 (br. s., 2H), 6.97-7.25 (m, 4H), 5.14 (br. s., 1H), 4.97 (br. s., 2H), 4.64 (q, J=7.28 Hz, 2H), 3.43 (br. s., 3H), 3.04 (dd, J=5.15, 16.19 Hz, 1H), 2.91 (br. s., 2H), 2.79 (s, 3H), 2.30 (s, 3H), 2.28 (s, 3H), 1.59 (t, J=7.28 Hz, 3H).

### 17. Preparation of (3S)-3-[7-(3,4-dimethylbenzoyl)-6,8-dihydro-5H-1,7-naphthyridin-2-yl]-3-(1-ethyl-4-methyl-benzotriazol-5-yl)propanoic acid

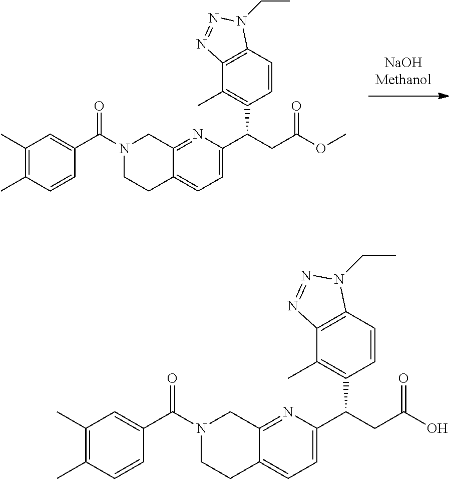

methyl (3S)-3-[7-(3,4-dimethylbenzoyl)-6,8-dihydro-5H-1,7-naphthyridin-2-yl]-3-(1-ethyl-4-methyl-benzotriazol-5-yl)propanoate (114.00 mg, 182.51 umol, Trifluoroacetate) in Methanol (2.00 mL) was added NaOH (2 M, 273.77 uL) and microwaved at 100° C. for 30 min. After neutralization with 2M HCl, the crude was purified with prep HPLC to give (3S)-3-[7-(3,4-dimethylbenzoyl)-6,8-dihydro-5H-1,7-naphthyridin-2-yl]-3-(1-ethyl-4-methyl-benzotriazol-5-yl)propanoic acid (63.20 mg, 120.66 umol, 66.11% yield, 95% purity). LCMS: Rt=1.24 min, m/z=498.2. ¹H NMR (400 MHz, CHLOROFORM-d) δ 9.30 (br. s., 2H), 7.61 (d, J=8.03 Hz, 1H), 7.34 (br. s., 2H), 6.97-7.25 (m, 4H), 5.14 (br. s., 1H), 4.97 (br. s., 2H), 4.64 (q, J=7.28 Hz, 2H), 3.43 (br. s., 3H), 3.04 (dd, J=5.15, 16.19 Hz, 1H), 2.91 (br. s., 2H), 2.79 (s, 3H), 2.30 (s, 3H), 2.28 (s, 3H), 1.59 (t, J=7.28 Hz, 3H).

### Example 136

### 3-(2-(3,4-dimethylbenzoyl)-1,2,3,4-tetrahydroisoquinolin-7-yl)-3-(1-ethyl-4-methyl-1H-benzo[d][1,2,3]triazol-5-yl)propanenitrile

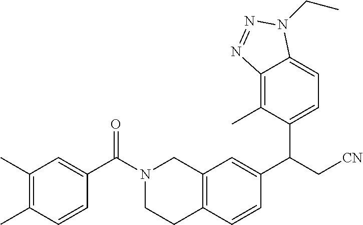 [SMILES: CCn1nnc2c(C)c(C(CC#N)c3ccc4c(c3)CN(C(=O)c3ccc(C)c(C)c3)CC4)ccc21]

### 1. Preparation of (E)-3-(1-ethyl-4-methyl-benzotriazol-5-yl)prop-2-enenitrile

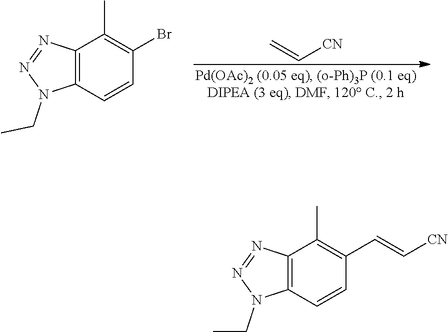

5-bromo-1-ethyl-4-methyl-benzotriazole (1.00 g, 4.16 mmol) prop-2-enenitrile (1.10 g, 20.80 mmol), Pd(OAc)₂ (93.40 mg, 416.00 umol), tris-o-tolylphosphane (253.24 mg, 832.00 umol) and DIPEA (1.61 g, 12.48 mmol, 2.18 mL) in DMF (8.00 mL) was microwaved at 120° C. for 2 h. After dilution with EtOAc and filtration through celite, the crude was washed with water and brine and dried through Na₂SO₄. The concentrated residue was chromatographed on Si gel (HE/EA 0-100%) to give (E)-3-(1-ethyl-4-methyl-benzotriazol-5-yl)prop-2-enenitrile (149.00 mg, 702.00 umol, 16.88% yield). LCMS: Rt=1.19 min, m/z=213.1.

### 2. 3-(2-(3,4-dimethylbenzoyl)-1,2,3,4-tetrahydroisoquinolin-7-yl)-3-(1-ethyl-4-methyl-1H-benzo[d][1,2,3]triazol-5-yl)propanenitrile

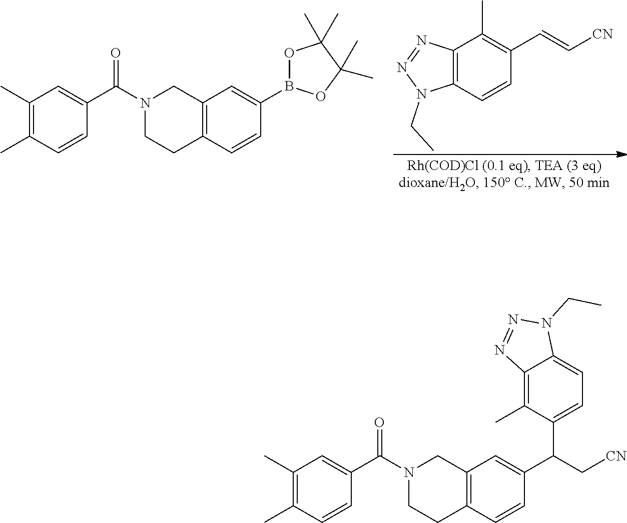

(3,4-dimethylphenyl)(7-(4,4,5,5-tetramethyl-1,3,2-dioxaborolan-2-yl)-3,4-dihydroisoquinolin-2(1H)-yl)methanone (403.75 mg, 1.03 mmol), (E)-3-(1-ethyl-4-methyl-1H-benzo[d][1,2,3]triazol-5-yl)acrylonitrile (146.00 mg, 687.87 umol), N,N-diethylethanamine (208.82 mg, 2.06 mmol, 287.24 uL) and chlororhodium;(1Z,5Z)-cycloocta-1,5-diene (33.92 mg, 68.79 umol) in Dioxane (1.50 mL) and water (500.00 uL) was microwaved at 150° C. for 50 min. After dilution with EtOAc and filtration through velite, the organic layer was washed with brine and dried over Na₂SO₄. The concentrated residue was chromatographed on Si gel (HE/EA 0-100%) to give 3-(2-(3,4-dimethylbenzoyl)-1,2,3,4-tetrahydroisoquinolin-7-yl)-3-(1-ethyl-4-methyl-1H-benzo[d][1,2,3]triazol-5-yl)propanenitrile (100.80 mg, 200.50 umol, 29.15% yield, 95% purity). LCMS: Rt=1.61 min, m/z=478.2. ¹H NMR (400 MHz, CHLOROFORM-d) δ 7.38 (br. s., 1H), 6.56-7.24 (m, 7H), 2.96-4.99 (m, 11H), 2.82 (s, 3H), 2.29 (d, J=6.78 Hz, 6H), 1.63 (t, J=7.28 Hz, 3H).

### Example 137

### 3-[2-(3,4-dimethylbenzoyl)-3,4-dihydro-1H-isoquinolin-7-yl]-3-(1-ethyl-4-methyl-benzotriazol-5-yl)-2-methyl-propanenitrile

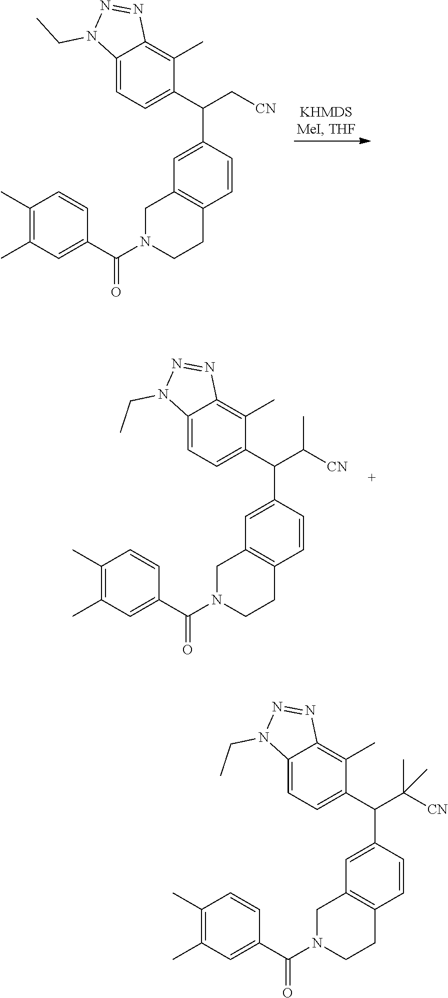 [SMILES: CCn1nnc2c(C)c(C(CC#N)c3ccc4c(c3)CN(C(=O)c3ccc(C)c(C)c3)CC4)ccc21 ; CCn1nnc2c(C)c(C(c3ccc4c(c3)CN(C(=O)c3ccc(C)c(C)c3)CC4)C(C)C#N)ccc21 ; CCn1nnc2c(C)c(C(c3ccc4c(c3)CN(C(=O)c3ccc(C)c(C)c3)CC4)C(C)(C)C#N)ccc21]

KHMDS (1 M, 258.79 uL) in THF (2.00 mL) was cooled to −78° C., and 3-[2-(3,4-dimethylbenzoyl)-3,4-dihydro-1H-isoquinolin-7-yl]-3-(1-ethyl-4-methyl-benzotriazol-5-yl)propanenitrile (103.00 mg, 215.66 umol) was added and stirred for 30 min, and warmed to −30° C. for 1 h. MeI (90.69 mg, 646.98 umol, 88.92 uL) was added and stirred to rt overnight. After quenched with water and extracted with EtOAc, the organic was dried over Na₂SO₄ and concentrated. The residue was purified with prep HPLC to give 3-[2-(3,4-dimethylbenzoyl)-3,4-dihydro-1H-isoquinolin-7-yl]-3-(1-ethyl-4-methyl-benzotriazol-5-yl)-2-methyl-propanenitrile (18.40 mg, 35.56 umol, 16.49% yield, 95% purity). LCMS: Rt=1.68 min, m/z 492.2. ¹H NMR (400 MHz, CHLOROFORM-d) δ 7.31-7.65 (m, 1H), 6.75-7.25 (m, 7H), 2.61-5.91 (m, 10H), 2.16-2.43 (m, 9H), 1.65 (br. s., 6H).

And 3-[2-(3,4-dimethylbenzoyl)-3,4-dihydro-1H-isoquinolin-7-yl]-3-(1-ethyl-4-methyl-benzotriazol-5-yl)-2,2-dimethyl-propanenitrile (6.00 mg, 11.27 umol, 5.23% yield, 95% purity). LCMS: Rt=1.77 min, m/z=506.2. ¹H NMR (400 MHz, CHLOROFORM-d) δ 7.34-7.54 (m, 2H), 6.81-7.24 (m, 6H), 2.44-5.10 (m, 9H), 2.29 (d, J=8.28 Hz, 9H), 1.23-1.88 (m, 9H).

### Example 138

### 3-[2-(3,4-dimethylbenzoyl)-3,4-dihydro-1H-isoquinolin-7-yl]-3-(6-methyl-1,3-benzodioxol-5-yl)propanoic acid

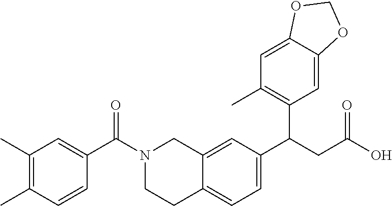 [SMILES: Cc1ccc(C(=O)N2CCc3ccc(C(CC(=O)O)c4cc5c(cc4C)OCO5)cc3C2)cc1C]

### 1. Preparation of methyl (E)-3-(6-methyl-1,3-benzodioxol-5-yl)prop-2-enoate

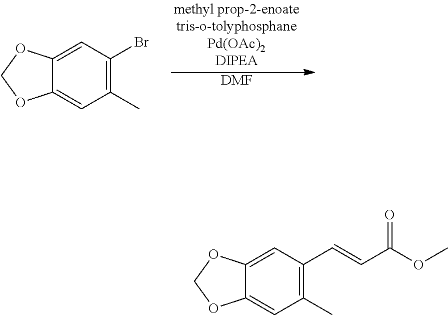

5-bromo-6-methyl-1,3-benzodioxole (774.00 mg, 3.60 mmol), methyl prop-2-enoate (1.55 g, 18.00 mmol, 1.61 mL), tris-o-tolylphosphane (219.11 mg, 720.00 umol), Pd(OAc)₂ (80.81 mg, 360.00 umol) and DIPEA (1.40 g, 10.80 mmol, 1.89 mL) in DMF (6.00 mL) was microwaved at 120° C. for 2 h. After filtration through celite and diluted with EtOAc and washing with water, brine and dried over Na₂SO₄, the concentrated residue was chromatographed on Si gel (HE/EA 0-100%) to give product methyl (E)-3-(6-methyl-1,3-benzodioxol-5-yl)prop-2-enoate (282.00 mg, 1.28 mmol, 35.57% yield). LCMS: Rt=1.48 min, m/z=221.2. ¹H NMR (400 MHz, CHLOROFORM-d) δ 7.92 (d, J=15.81 Hz, 1H), 7.04 (s, 1H), 6.68 (s, 1H), 6.22 (d, J=15.81 Hz, 1H), 5.97 (s, 2H), 3.81 (s, 3H), 2.38 (s, 3H).

### 2. Preparation of methyl 3-[2-(3,4-dimethylbenzoyl)-3,4-dihydro-1H-isoquinolin-7-yl]-3-(6-methyl-1,3-benzodioxol-5-yl)propanoate

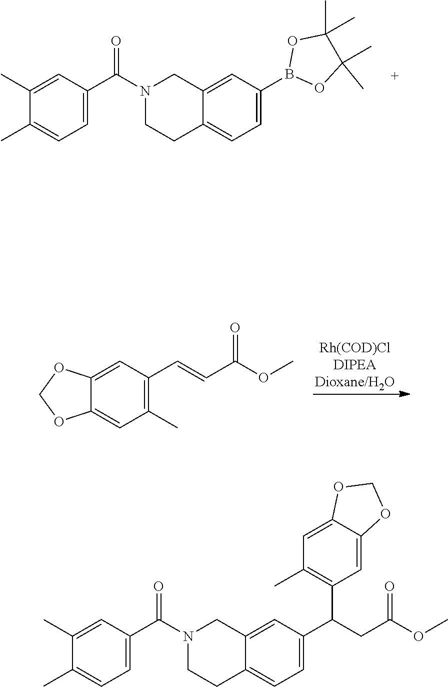

(3,4-dimethylphenyl)-[7-(4,4,5,5-tetramethyl-1,3,2-dioxaborolan-2-yl)-3,4-dihydro-1H-isoquinolin-2-yl]methanone (498.42 mg, 1.27 mmol), methyl (E)-3-(6-methyl-1,3-benzodioxol-5-yl)prop-2-enoate (187.00 mg, 849.15 umol), N,N-diethylethanamine (257.78 mg, 2.55 mmol, 353.12 uL), chlororhodium;(1Z,5Z)-cycloocta-1,5-diene (41.87 mg, 84.92 umol) in Dioxane (1.50 mL) and water (500.00 uL) was microwaved at 150° C. for 50 min. After dilution with EtOAc and filtration through celite, the organic phase was washed with brine and dried over Na₂SO₄. The concentrated residue was chromatographed on Si gel (HE/EA 0-100%) to give methyl 3-[2-(3,4-dimethylbenzoyl)-3,4-dihydro-1H-isoquinolin-7-yl]-3-(6-methyl-1,3-benzodioxol-5-yl)propanoate (293.00 mg, 603.41 umol, 71.06% yield). LCMS: Rt=1.86 min, m/z=486.2.

### 3. Preparation of 3-[2-(3,4-dimethylbenzoyl)-3,4-dihydro-1H-isoquinolin-7-yl]-3-(6-methyl-1,3-benzodioxol-5-yl)propanoic acid

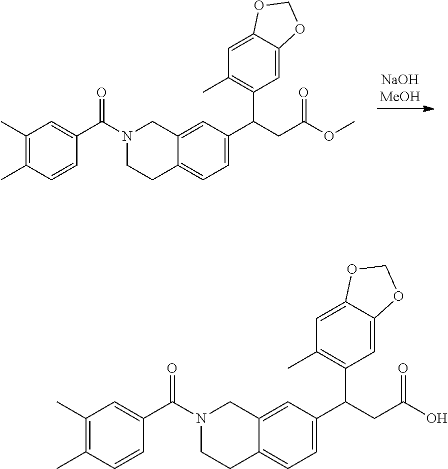

methyl 3-[2-(3,4-dimethylbenzoyl)-3,4-dihydro-1H-isoquinolin-7-yl]-3-(6-methyl-1,3-benzodioxol-5-yl)propanoate (103.00 mg, 212.12 umol) in Methanol (2.00 mL) was added NaOH (2 M, 212.12 uL) and microwaved at 100° C. for 30 min. After neutralization with 2M HCl, the crude was purified with prep HPLC to give 3-[2-(3,4-dimethylbenzoyl)-3,4-dihydro-1H-isoquinolin-7-yl]-3-(6-methyl-1,3-benzodioxol-5-yl)propanoic acid (48.80 mg, 98.32 umol, 46.35% yield, 95% purity). LCMS: Rt=1.64 min, m/z=472.2. ¹H NMR (400 MHz, CHLOROFORM-d) δ 6.29-7.24 (m, 8H), 5.91 (br. s., 2H), 2.60-5.14 (m, 9H), 2.12-2.43 (m, 9H).

### Examples 139 and 140

### (3S)-3-[2-(3,4-dimethylbenzoyl)-3,4-dihydro-1H-isoquinolin-7-yl]-3-(6-methyl-1,3-benzodioxol-5-yl)propanoic acid and (3R)-3-[2-(3,4-dimethylbenzoyl)-3,4-dihydro-1H-isoquinolin-7-yl]-3-(6-methyl-1,3-benzodioxol-5-yl)propanoic acid

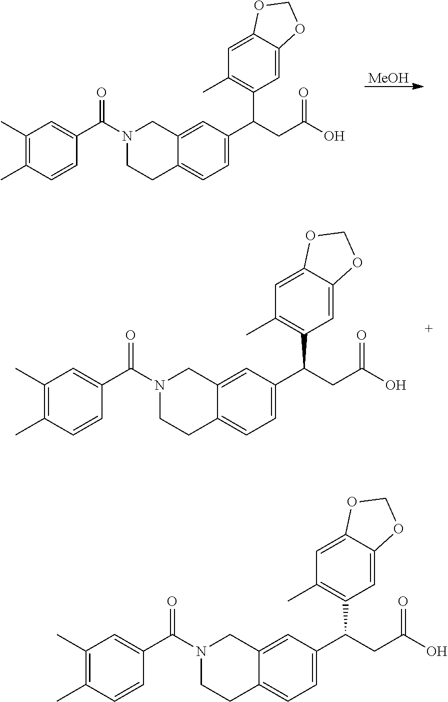 [SMILES: "MeOH" ; Cc1ccc(C(=O)N2CCc3ccc(C(CC(=O)O)c4cc5c(cc4C)OCO5)cc3C2)cc1C ; Cc1ccc(C(=O)N2CCc3ccc([C@H](CC(=O)O)c4cc5c(cc4C)OCO5)cc3C2)cc1C ; Cc1ccc(C(=O)N2CCc3ccc([C@@H](CC(=O)O)c4cc5c(cc4C)OCO5)cc3C2)cc1C]

3-[2-(3,4-dimethylbenzoyl)-3,4-dihydro-1H-isoquinolin-7-yl]-3-(6-methyl-1,3-benzodioxol-5-yl)propanoic acid (43.00 mg, 91.19 umol) was separated under the following SFC condition (Column: CHIRALPAK IA 30×250 mm, 5 um; Method: 50% Methanol w/0.1% DEA in CO₂ (flow rate: 100 mL/min), ABPR 120 bar, MBPR 40 psi) to give (3S)-3-[2-(3,4-dimethylbenzoyl)-3,4-dihydro-1H-isoquinolin-7-yl]-3-(6-methyl-1,3-benzodioxol-5-yl)propanoic acid (15.00 mg, 27.54 umol, 30.20% yield, 100% purity, N-ethylethanamine). LCMS: Rt=1.64 min, m/z=472.2. ¹H NMR (400 MHz, CHLOROFORM-d) δ 6.37-7.25 (m, 8H), 5.86 (s, 2H), 2.41-5.06 (m, 13H), 2.30 (s, 6H), 2.20 (br. s., 3H), 1.08 (s, 6H).

And (3R)-3-[2-(3,4-dimethylbenzoyl)-3,4-dihydro-1H-isoquinolin-7-yl]-3-(6-methyl-1,3-benzodioxol-5-yl)propanoic acid (13.00 mg, 23.87 umol, 26.18% yield, 100% purity, N-ethylethanamine). LCMS: Rt=1.64 min, m/z=472.2. ¹H NMR (400 MHz, CHLOROFORM-d) δ 6.37-7.25 (m, 8H), 5.86 (s, 2H), 2.41-5.06 (m, 13H), 2.30 (s, 6H), 2.20 (br. s., 3H), 1.08 (s, 6H).

### Example 141

### 3-(2-(3,4-dimethylbenzoyl)-1,2,3,4-tetrahydroisoquinolin-7-yl)-3-(7-methyl-2,3-dihydrobenzo[b][1,4]dioxin-6-yl)propanoic acid

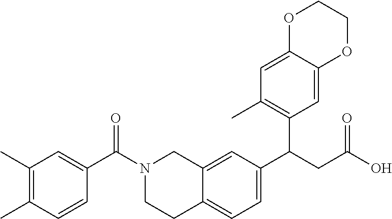 [SMILES: Cc1ccc(C(=O)N2CCc3ccc(C(CC(=O)O)c4cc5c(cc4C)OCCO5)cc3C2)cc1C]

3-(2-(3,4-dimethylbenzoyl)-1,2,3,4-tetrahydroisoquinolin-7-yl)-3-(7-methyl-2,3-dihydrobenzo[b][1,4]dioxin-6-yl)propanoic acid was prepared following the procedure as Example 138, and was purified with prep HPLC to give 3-[2-(3,4-dimethylbenzoyl)-3,4-dihydro-1H-isoquinolin-7-yl]-3-(7-methyl-2,3-dihydro-1,4-benzodioxin-6-yl)propanoic acid (50.80 mg, 99.39 umol, 51.05% yield, 95% purity). LCMS: Rt=1.61 min, m/z=486.2. ¹H NMR (400 MHz, CHLOROFORM-d) δ 7.12-7.25 (m, 3H), 6.44-7.11 (m, 5H), 4.36-5.13 (m, 1H), 4.23 (br. s., 4H), 2.55-3.89 (m, 8H), 2.31 (s, 6H), 2.18 (br. s., 3H).

### Example 142

### (3S)-3-[2-(3,4-dimethylbenzoyl)-3,4-dihydro-1H-isoquinolin-7-yl]-3-(7-methyl-2,3-dihydro-1,4-benzodioxin-6-yl)propanoic acid

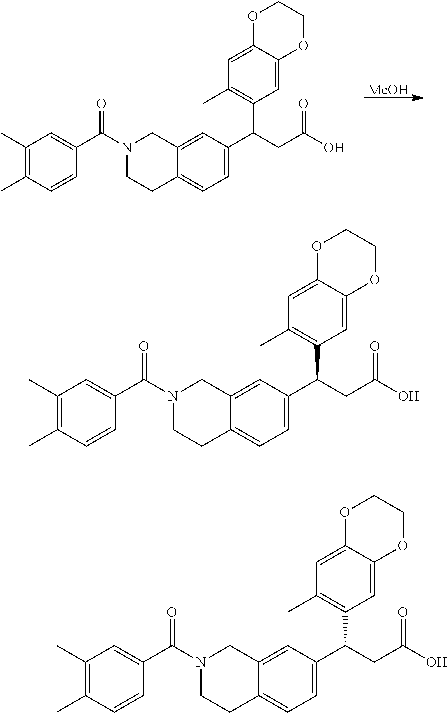 [SMILES: "MeOH" ; Cc1ccc(C(=O)N2CCc3ccc(C(CC(=O)O)c4cc5c(cc4C)OCCO5)cc3C2)cc1C ; Cc1ccc(C(=O)N2CCc3ccc([C@H](CC(=O)O)c4cc5c(cc4C)OCCO5)cc3C2)cc1C ; Cc1ccc(C(=O)N2CCc3ccc([C@@H](CC(=O)O)c4cc5c(cc4C)OCCO5)cc3C2)cc1C]

3-[2-(3,4-dimethylbenzoyl)-3,4-dihydro-1H-isoquinolin-7-yl]-3-(7-methyl-2,3-dihydro-1,4-benzodioxin-6-yl)propanoic acid (42.00 mg, 86.50 umol) was separated under the following SFC condition (Column: CHIRALPAK OX-H 30×250 mm, 5 um; Method: 45% Ethanol w/0.1% DEA in CO₂ (flow rate: 100 mL/min), ABPR 120 bar, MBPR 40 psi) to give (3S)-3-[2-(3,4-dimethylbenzoyl)-3,4-dihydro-1H-isoquinolin-7-yl]-3-(7-methyl-2,3-dihydro-1,4-benzodioxin-6-yl)propanoic acid (15.60 mg, 26.53 umol, 30.67% yield, 95% purity, N-ethylethanamine). LCMS: Rt=1.62 min, m/z=486.2. ¹H NMR (400 MHz, CHLOROFORM-d) δ 6.34-7.23 (m, 9H), 2.68-5.09 (m, 14H), 2.56 (br. s., 4H), 2.29 (d, J=6.02 Hz, 6H), 2.15 (br. s., 3H), 0.92-1.17 (m, 6H).

and (3R)-3-[2-(3,4-dimethylbenzoyl)-3,4-dihydro-1H-isoquinolin-7-yl]-3-(7-methyl-2,3-dihydro-1,4-benzodioxin-6-yl)propanoic acid (15.40 mg, 26.19 umol, 30.27% yield, 95% purity, N-ethylethanamine). LCMS: Rt=1.62 min, m/z=486.2. ¹H NMR (400 MHz, CHLOROFORM-d) δ 6.34-7.23 (m, 9H), 2.68-5.09 (m, 14H), 2.56 (br. s., 4H), 2.29 (d, J=6.02 Hz, 6H), 2.15 (br. s., 3H), 0.92-1.17 (m, 6H).

### Example 143

### 3-(2-(3,4-dimethylbenzoyl)-1,2,3,4-tetrahydroisoquinolin-7-yl)-3-(4-methylbenzo[d][1,3]dioxol-5-yl)propanoic acid

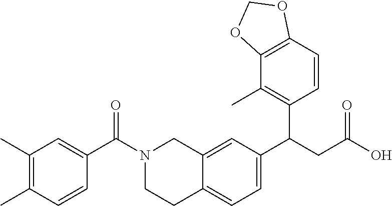 [SMILES: Cc1ccc(C(=O)N2CCc3ccc(C(CC(=O)O)c4ccc5c(c4C)OCO5)cc3C2)cc1C]

3-(2-(3,4-dimethylbenzoyl)-1,2,3,4-tetrahydroisoquinolin-7-yl)-3-(4-methylbenzo[d][1,3]dioxol-5-yl)propanoic acid was prepared following the procedure as Example 138, and was purified with prep HPLC to give 3-[2-(3,4-dimethylbenzoyl)-3,4-dihydro-1H-isoquinolin-7-yl]-3-(4-methyl-1,3-benzodioxol-5-yl)propanoic acid (28.20 mg, 56.81 umol, 45.98% yield, 95% purity). LCMS: Rt=1.67 min, m/z=472.2. ¹H NMR (400 MHz, CHLOROFORM-d) δ 6.53-7.25 (m, 8H), 5.92 (d, J=5.52 Hz, 2H), 2.63-5.07 (m, 9H), 2.30 (d, J=9.04 Hz, 6H), 2.13 (br. s., 3H).

### Examples 144 and 145

### (3S)-3-[2-(3,4-dimethylbenzoyl)-3,4-dihydro-1H-isoquinolin-7-yl]-3-(4-methyl-1,3-benzodioxol-5-yl)propanoic acid and (3R)-3-[2-(3,4-dimethylbenzoyl)-3,4-dihydro-1H-isoquinolin-7-yl]-3-(4-methyl-1,3-benzodioxol-5-yl)propanoic acid

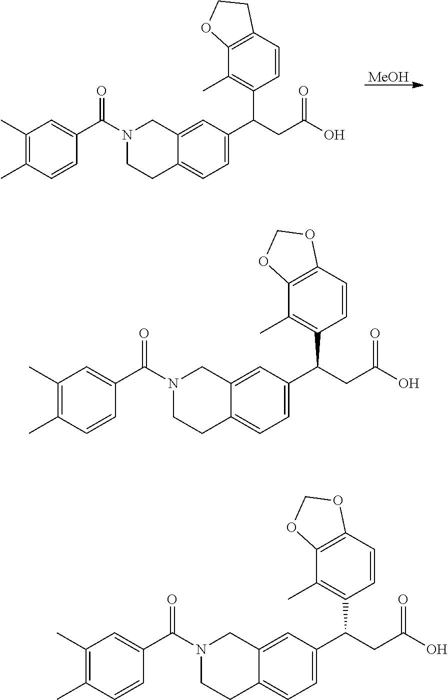 [SMILES: "MeOH" ; Cc1ccc(C(=O)N2CCc3ccc(C(CC(=O)O)c4ccc5c(c4C)OCC5)cc3C2)cc1C ; Cc1ccc(C(=O)N2CCc3ccc([C@H](CC(=O)O)c4ccc5c(c4C)OCO5)cc3C2)cc1C ; Cc1ccc(C(=O)N2CCc3ccc([C@@H](CC(=O)O)c4ccc5c(c4C)OCO5)cc3C2)cc1C]

3-[2-(3,4-dimethylbenzoyl)-3,4-dihydro-1H-isoquinolin-7-yl]-3-(4-methyl-1,3-benzodioxol-5-yl)propanoic acid (24.00 mg, 50.90 umol) was separated with the following SFC condition (Column: CHIRALPAK OX-H 30×250 mm, 5 um; Method: 40% Methanol with 0.1% DEA in CO2 (flow rate: 100 mL/min), ABPR 120 bar, MBPR 40 psi) to give (3S)-3-[2-(3,4-dimethylbenzoyl)-3,4-dihydro-1H-isoquinolin-7-yl]-3-(4-methyl-1,3-benzodioxol-5-yl)propanoic acid (5.00 mg, 8.72 umol, 17.13% yield, 95% purity, N-ethylethanamine). LCMS: Rt=1.65 min, m/z=472.2. ¹H NMR (400 MHz, CHLOROFORM-d) δ 6.45-7.25 (m, 8H), 5.86 (br. s., 2H), 3.36-4.93 (m, 5H), 2.79 (br. s., 4H), 2.57 (d, J=6.53 Hz, 4H), 2.28 (d, J=7.78 Hz, 6H), 2.10 (br. s., 3H), 1.05 (t, J=7.15 Hz, 6H).

And (3R)-3-[2-(3,4-dimethylbenzoyl)-3,4-dihydro-1H-isoquinolin-7-yl]-3-(4-methyl-1,3-benzodioxol-5-yl)propanoic acid (5.20 mg, 9.07 umol, 17.82% yield, 95% purity, N-ethylethanamine). LCMS: Rt=1.65 min, m/z=472.2. ¹H NMR (400 MHz, CHLOROFORM-d) δ 6.45-7.25 (m, 8H), 5.86 (br. s., 2H), 3.36-4.93 (m, 5H), 2.79 br. s., 4H), 2.57 (d, J=6.53 Hz, 4H), 2.28 (d, J=7.78 Hz, 6H), 2.10 (br. s., 3H), 1.05 (t, J=7.15 Hz, 6H).

### Example 146

### 3-(2-(3,4-dimethylbenzoyl)-1,2,3,4-tetrahydroisoquinolin-7-yl)-3-(7-methylchroman-6-yl)propanoic acid

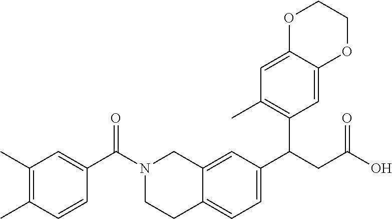 [SMILES: Cc1ccc(C(=O)N2CCc3ccc(C(CC(=O)O)c4cc5c(cc4C)OCCO5)cc3C2)cc1C]

3-(2-(3,4-dimethylbenzoyl)-1,2,3,4-tetrahydroisoquinolin-7-yl)-3-(7-methylchroman-6-yl)propanoic acid was prepared following the procedure as Example 138, and was purified with prep HPLC to give 3-(2-(3,4-dimethylbenzoyl)-1,2,3,4-tetrahydroisoquinolin-7-yl)-3-(7-methylchroman-6-yl)propanoic acid (137 mg, yield 40%). LCMS: Rt=1.69 min, m/z=484.2. ¹H NMR (400 MHz, CHLOROFORM-d) δ 8.18 (br. s., 1H), 6.44-7.25 (m, 8H), 2.60-5.08 (m, 13H), 2.31 (s, 6H), 2.20 (br. s., 3H), 2.00 (d, J=9.79 Hz, 2H).

### Example 147

### 3-[2-(3,4-dimethylbenzoyl)-3,4-dihydro-1H-isoquinolin-7-yl]-3-(2-methyl-3,4-dihydro-1H-isoquinolin-6-yl)propanoic acid

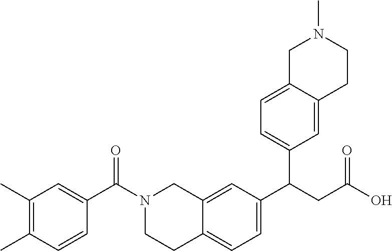 [SMILES: Cc1ccc(C(=O)N2CCc3ccc(C(CC(=O)O)c4ccc5c(c4)CCN(C)C5)cc3C2)cc1C]

3-[2-(3,4-dimethylbenzoyl)-3,4-dihydro-1H-isoquinolin-7-yl]-3-(2-methyl-3,4-dihydro-1H-isoquinolin-6-yl)propanoic acid was prepared following the procedure as Example 138, and was purified with prep HPLC to give 3-[2-(3,4-dimethylbenzoyl)-3,4-dihydro-1H-isoquinolin-7-yl]-3-(2-methyl-3,4-dihydro-1H-isoquinolin-6-yl)propanoic acid (8.20 mg, 13.08 umol, 20.49% yield, 95% purity, Trifluoroacetate). LCMS: Rt=1.11 min, m/z=483.2. ¹H NMR (400 MHz, CHLOROFORM-d) δ 6.74-7.25 (m, 9H), 2.70-4.96 (m, 19H), 2.29 (d, J=8.28 Hz, 6H).

### Example 148

### 3-[2-(3,4-dimethylbenzoyl)-3,4-dihydro-1H-isoquinolin-7-yl]-3-(6-fluoro-1-methyl-benzimidazol-5-yl)propanoic acid

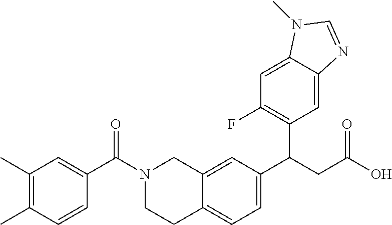 [SMILES: Cc1ccc(C(=O)N2CCc3ccc(C(CC(=O)O)c4cc5ncn(C)c5cc4F)cc3C2)cc1C]

3-[2-(3,4-dimethylbenzoyl)-3,4-dihydro-1H-isoquinolin-7-yl]-3-(6-fluoro-1-methyl-benzimidazol-5-yl)propanoic acid was prepared following the procedure as Example 138, and was purified with prep HPLC to give 3-[2-(3,4-dimethylbenzoyl)-3,4-dihydro-1H-isoquinolin-7-yl]-3-(6-fluoro-1-methyl-benzimidazol-5-yl)propanoic acid (38.00 mg, 74.35 umol, 41.13% yield, 95% purity). LCMS: Rt=1.13 min, m/z=486.2. ¹H NMR (400 MHz, DMSO-d6) δ 9.20 (br. s., 1H), 7.66-8.04 (m, 2H), 6.93-7.38 (m, 6H), 4.39-4.88 (m, 3H), 3.93 (s, 3 H), 3.30-3.90 (br. s., 3H), 3.11 (br. s., 2H), 2.76 (br. s., 2H), 2.24 (d, J=6.78 Hz, 6H).

### Example 149

### (3S)-3-[2-(3,4-dimethylbenzoyl)-3,4-dihydro-1H-isoquinolin-7-yl]-3-(6-fluoro-1-methyl-benzimidazol-5-yl)propanoic acid and

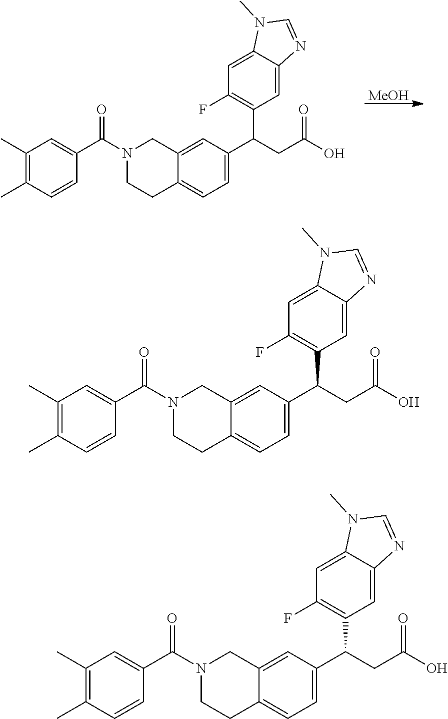 [SMILES: "MeOH" ; Cc1ccc(C(=O)N2CCc3ccc(C(CC(=O)O)c4cc5ncn(C)c5cc4F)cc3C2)cc1C ; Cc1ccc(C(=O)N2CCc3ccc([C@H](CC(=O)O)c4cc5ncn(C)c5cc4F)cc3C2)cc1C ; Cc1ccc(C(=O)N2CCc3ccc([C@@H](CC(=O)O)c4cc5ncn(C)c5cc4F)cc3C2)cc1C]

3-[2-(3,4-dimethylbenzoyl)-3,4-dihydro-1H-isoquinolin-7-yl]-3-(6-fluoro-1-methyl-benzimidazol-5-yl)propanoic acid (33.00 mg, 67.96 umol) was separated under the following SFC condition (Column: CHIRALPAK IA 30×250 mm, 5 um; Method: 50% Methanol w/0.1% DEA in CO₂ (flow rate: 100 mL/min), ABPR 120 bar, MBPR 40 psi) to give peak1 (3S)-3-[2-(3,4-dimethylbenzoyl)-3,4-dihydro-1H-isoquinolin-7-yl]-3-(6-fluoro-1-methyl-benzimidazol-5-yl)propanoic acid (9.20 mg, 15.64 umol, 23.02% yield, 95% purity, N-ethylethanamine). LCMS: Rt=1.12 min, m/z=486.2; ¹H NMR (400 MHz, CHLOROFORM-d) δ 7.61-7.93 (m, 2H), 6.82-7.24 (m, 6H), 3.84-5.16 (m, 3H), 3.75 (s, 3H), 2.71-3.65 (m, 6H), 2.57 (br. s., 4H), 2.28 (d, J=6.78 Hz, 6H), 1.01 (d, J=14.31 Hz, 6H).

and peak2 (3R)-3-[2-(3,4-dimethylbenzoyl)-3,4-dihydro-1H-isoquinolin-7-yl]-3-(6-fluoro-1-methyl-benzimidazol-5-yl)propanoic acid (9.30 mg, 15.81 umol, 23.27% yield, 95% purity, N-ethylethanamine). LCMS: Rt=1.12 min, m/z=486.2. ¹H NMR (400 MHz, CHLOROFORM-d) δ 7.61-7.93 (m, 2H), 6.82-7.24 (m, 6H), 3.84-5.16 (m, 3H), 3.75 (s, 3H), 2.71-3.65 (m, 6H), 2.57 (br. s., 4H), 2.28 (d, J=6.78 Hz, 6H), 1.01 (d, J=14.31 Hz, 6H).

### Example 150

### 3-[2-(3,4-dimethylbenzoyl)-3,4-dihydro-1H-isoquinolin-7-yl]-3-(7-methylquinoxalin-6-yl)propanoic acid

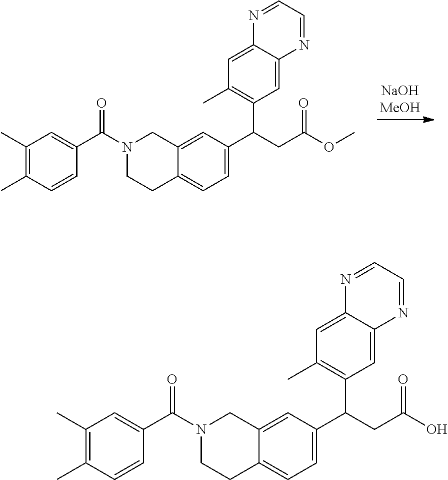

3-[2-(3,4-dimethylbenzoyl)-3,4-dihydro-1H-isoquinolin-7-yl]-3-(7-methylquinoxalin-6-yl)propanoic acid was prepared following the procedure as Example 138, and was purified with prep HPLC to give 3-[2-(3,4-dimethylbenzoyl)-3,4-dihydro-1H-isoquinolin-7-yl]-3-(7-methylquinoxalin-6-yl)propanoic acid (169.00 mg, 352.40 umol, 54.87% yield). LCMS: Rt=1.42 min, m/z=480.2. ¹H NMR (400 MHz, CHLOROFORM-d) δ 8.86 (br. s., 2H), 8.17 (br. s., 1H), 7.90 (br. s., 1H), 6.44-7.25 (m, 6H), 4.26-5.08 (m, 3H), 2.63-4.13 (m, 6H), 2.47 (br. s., 3H), 2.29 (s, 6H).

### Example 151

### (S)—N-(2-(2-(3,4-dimethylbenzoyl)-1,2,3,4-tetrahydroisoquinolin-7-yl)-2-(1-ethyl-4-methyl-1H-benzo[d][1,2,3]triazol-5-yl)ethyl)methanesulfonamide

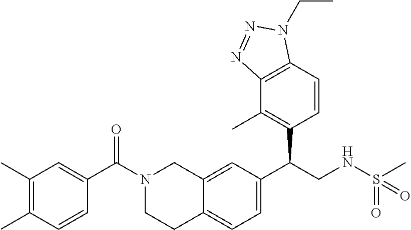 [SMILES: CCn1nnc2c(C)c([C@@H](CNS(C)(=O)=O)c3ccc4c(c3)CN(C(=O)c3ccc(C)c(C)c3)CC4)ccc21]

### 1. Preparation of [7-[(1S)-2-amino-1-(1-ethyl-4-methyl-benzotriazol-5-yl)ethyl]-3,4-dihydro-1H-isoquinolin-2-yl]-(3,4-dimethylphenyl)methanone

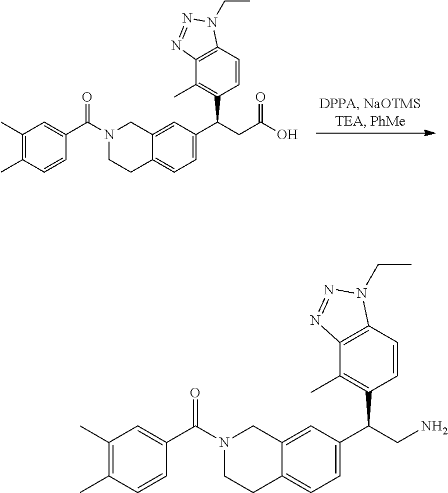 [SMILES: CCn1nnc2c(C)c([C@@H](CC(=O)O)c3ccc4c(c3)CN(C(=O)c3ccc(C)c(C)c3)CC4)ccc21 ; CCn1nnc2c(C)c([C@@H](CN)c3ccc4c(c3)CN(C(=O)c3ccc(C)c(C)c3)CC4)ccc21]

(3S)-3-[2-(3,4-dimethylbenzoyl)-3,4-dihydro-1H-isoquinolin-7-yl]-3-(1-ethyl-4-methyl-benzotriazol-5-yl)propanoic acid (212.90 mg, 428.72 umol) in Toluene (4.00 mL) was added DPPA (117.98 mg, 428.72 umol, 92.17 uL) and TEA (52.06 mg, 514.46 umol, 71.32 uL) and refluxed for 3 h. After cooled to 0° C., NaOTMS (1 M, 857.44 uL) was added and stirred for 2 h. The crude was acidified with citric acid and purified with Prep HPLC to give [7-[(1S)-2-amino-1-(1-ethyl-4-methyl-benzotriazol-5-yl)ethyl]-3,4-dihydro-1H-isoquinolin-2-yl]-(3,4-dimethylphenyl)methanone (166 mg, yield 78%). LCMS: Rt=1.15 min, m/z=468.2. ¹H NMR (400 MHz, CHLOROFORM-d) δ 6.70-7.23 (m, 8H), 2.69-4.91 (m, 11H), 2.61 (br. s., 3H), 2.09-2.38 (m, 6H), 1.56 (br. s., 3H).

### 2. Preparation of N-[(2S)-2-[2-(3,4-dimethylbenzoyl)-3,4-dihydro-1H-isoquinolin-7-yl]-2-(1-ethyl-4-methyl-benzotriazol-5-yl)ethyl]methanesulfonamide

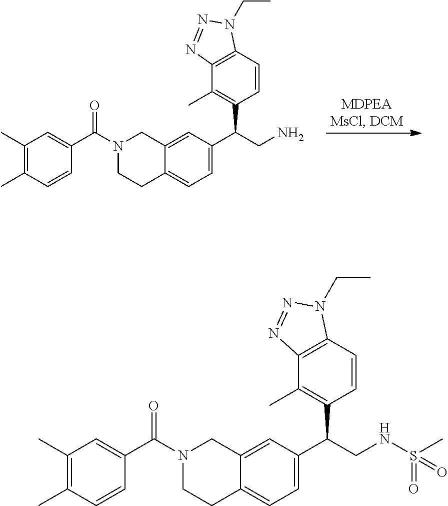 [SMILES: CCn1nnc2c(C)c([C@@H](CN)c3ccc4c(c3)CN(C(=O)c3ccc(C)c(C)c3)CC4)ccc21 ; CCn1nnc2c(C)c([C@@H](CNS(C)(=O)=O)c3ccc4c(c3)CN(C(=O)c3ccc(C)c(C)c3)CC4)ccc21]

[7-[(1S)-2-amino-1-(1-ethyl-4-methyl-benzotriazol-5-yl)ethyl]-3,4-dihydro-1H-isoquinolin-2-yl]-(3,4-dimethylphenyl)methanone (30.00 mg, 64.16 umol) in DCM (2.00 mL) was added DIPEA (24.88 mg, 192.48 umol, 33.62 uL) and methanesulfonyl chloride (11.02 mg, 96.24 umol, 7.45 uL) and stirred at rt for overnight. The crude was purified on Si gel (HE/EA 0-100%) to give N-[(2S)-2-[2-(3,4-dimethylbenzoyl)-3,4-dihydro-1H-isoquinolin-7-yl]-2-(1-ethyl-4-methyl-benzotriazol-5-yl)ethyl]methanesulfonamide (29.20 mg, 50.83 umol, 79.23% yield, 95% purity). LCMS: Rt=1.51 min, m/z=546.2. ¹H NMR (400 MHz, CHLOROFORM-d) δ 7.37 (br. s., 2H), 6.83-7.24 (m, 6H), 4.19-5.06 (m, 6H), 3.36-4.09 (m, 4H), 2.66-3.13 (m, 7H), 2.29 (d, J=7.03 Hz, 6H), 1.62 (d, J=14.31 Hz, 3H).

### Example 152

### N-[(2S)-2-[2-(3,4-dimethylbenzoyl)-3,4-dihydro-1H-isoquinolin-7-yl]-2-(1-ethyl-4-methyl-benzotriazol-5-yl)ethyl]formamide

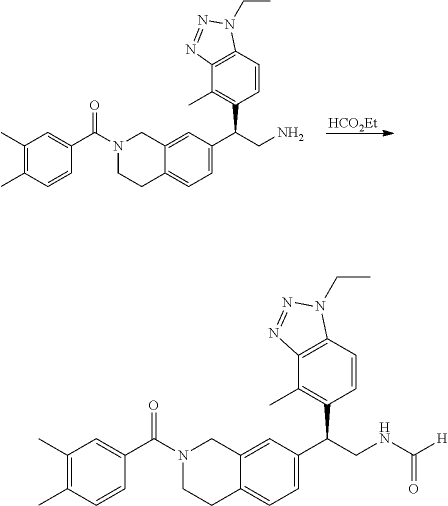 [SMILES: CCn1nnc2c(C)c([C@@H](CN)c3ccc4c(c3)CN(C(=O)c3ccc(C)c(C)c3)CC4)ccc21 ; CCn1nnc2c(C)c([C@@H](CNC=O)c3ccc4c(c3)CN(C(=O)c3ccc(C)c(C)c3)CC4)ccc21 ; "HCO2Et"]

[7-[(1S)-2-amino-1-(1-ethyl-4-methyl-benzotriazol-5-yl)ethyl]-3,4-dihydro-1H-isoquinolin-2-yl]-(3,4-dimethylphenyl)methanone (66.00 mg, 141.15 umol) in ethyl formate (1.84 g, 24.84 mmol, 2.00 mL) was refluxed overnight. After concentration, the crude was chromatographed on Si gel (DCM/MeOH 0-100%) to give N-[(2S)-2-[2-(3,4-dimethylbenzoyl)-3,4-dihydro-1H-isoquinolin-7-yl]-2-(1-ethyl-4-methyl-benzotriazol-5-yl)ethyl]formamide (24.70 mg, 47.35 umol, 33.54% yield, 95% purity). LCMS: RT=1.40 min, m/z=496.2. ¹H NMR (400 MHz, CHLOROFORM-d) δ 8.07 (d, J=15.81 Hz, 1H), 7.37 (br. s., 2H), 6.83-7.25 (m, 6H), 3.53-4.98 (m, 9H), 2.79 (br. s., 5H), 2.29 (d, J=8.03 Hz, 6H), 1.61 (t, J=7.28 Hz, 3H).

### Examples 153 and 154

### (3S)-3-(10-benzoyl-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-2-C-1) and (3R)-3-(10-benzoyl-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-2-C-2)

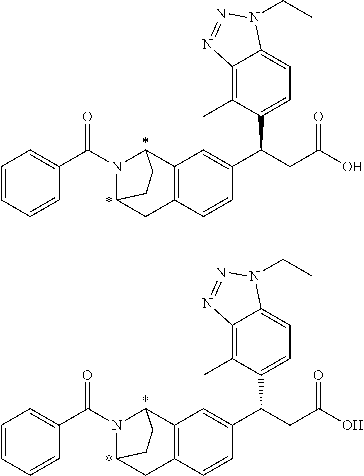 [SMILES: CCn1nnc2c(C)c([C@@H](CC(=O)O)c3ccc4c(c3)C3CCC(C4)N3C(=O)c3ccccc3)ccc21 ; CCn1nnc2c(C)c([C@H](CC(=O)O)c3ccc4c(c3)C3CCC(C4)N3C(=O)c3ccccc3)ccc21]

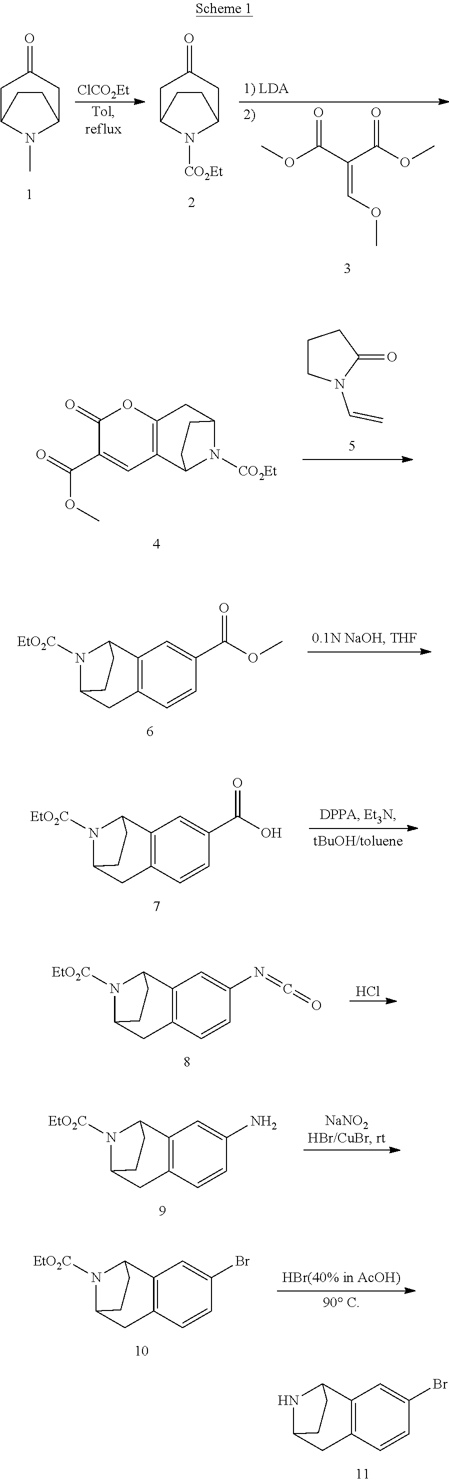

### 1. Preparation of ethyl 3-oxo-8-azabicyclo[3.2.1]octane-8-carboxylate

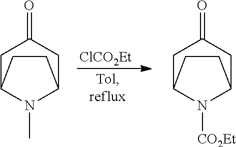

To a solution of compound 8-methyl-8-azabicyclo[3.2.1]octan-3-one (75 g, 0.54 mol, 1.0 eq) in toluene (750 mL) was added compound ethyl carbonochloridate (117 g, 103.5 mL, 1.08 mol, 1.0 eq) by dropwise at 23° C., and then K₂CO₃ (745 mg, 5.4 mmol, 0.01 eq). The resulting mixture was heated to reflux and stirred for 3 h. The reaction mixture was concentrated under reduced pressure. The residue was diluted with EtOAc (800 mL), washed with water (300 mL×3). The organic layer was dried over Na₂SO₄, filtered, concentrated and purified by column chromatography on silica gel (PE:EA=40:1-30:1-20:1-10:1) to supply ethyl 3-oxo-8-azabicyclo[3.2.1]octane-8-carboxylate (95 g, 89.6% yield) as pale-yellow liquid. ¹HNMR (400 MHz, CDCl₃) δ: 4.54 (br s, 2H), 4.19 (q, J=7.0 Hz, 2H), 2.66 (br s, 2H), 2.34 (d, J=16.0 Hz, 2H), 2.14-2.04 (m, 2H), 1.71-1.62 (m, 2H),1.29 (t, J=7.0 Hz, 3H).

### 2. Preparation of 10-ethyl 3-methyl 2-oxo-2,5,6,7,8,9-hexahydro-5,8-epiminocyclohepta[b]pyran-3,10-dicarboxylate

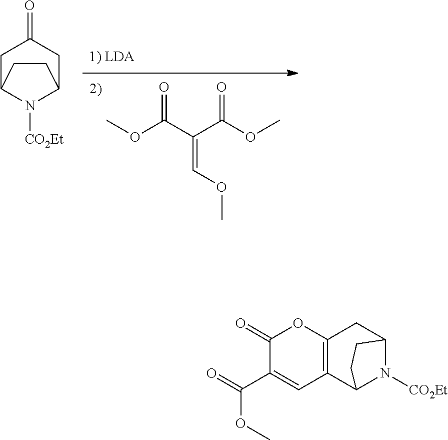

To the solution of compound LDA (0.285 L, 0.571 mol, 1.23 eq, 2 M in THF/hexane) in 1.4 L of THF was added ethyl 3-oxo-8-azabicyclo[3.2.1]octane-8-carboxylate (85 g, 0.464 mol, 1.0 eq) in THF (0.2 L) by dropwise at −70° C. After addition, the mixture was stirred at −70° C.˜-10° C. for 1.5 h, dimethyl 2-(methoxymethylene)malonate (89.7 g, 0.516 mol, 1.11 eq) in THF (0.25 L) was added at −70° C. After addition, the mixture was warmed to room temperature slowly and stirred for 16 h. The mixture was poured into 1.5 L of saturated aqueous NH₄Cl; adjust Ph to ˜5 by adding 2N HCl aq. The organic layer was separated and the aqueous phase was extracted with EtOAc (500 mL×5). The combined organic layers were washed with brine (500 mL), dried over Na₂SO₄, filtered, concentrated and purified by column chromatography on silica gel (PE:EA=15:1-12:1-8:1-4:1-2:1) to supply 10-ethyl 3-methyl 2-oxo-2,5,6,7,8,9-hexahydro-5,8-epiminocyclohepta[b]pyran-3,10-dicarboxylate (51 g, 35.9% yield) as pale-yellow solid. ¹HNMR: (400 MHz, CDCl₃) δ: 8.08 (s, 1H), 4.84 (br s, 1H), 4.61 (br s, 1H), 4.16 (q, J=7.0 Hz, 2H), 3.91 (s, 3H), 3.24 (br s, 1H), 2.45-2.30 (m, 2H), 2.19 (m, 1H), 1.97 (m, 1H),1.73 (m, 1H), 1.61-1.56 (m, 1H), 1.26 (t, J=7.0 Hz, 3H).

### 3. Preparation of 10-ethyl 3-methyl 6,7,8,9-tetrahydro-5H-5,8-epiminobenzo[7]annulene-3,10-dicarboxylate

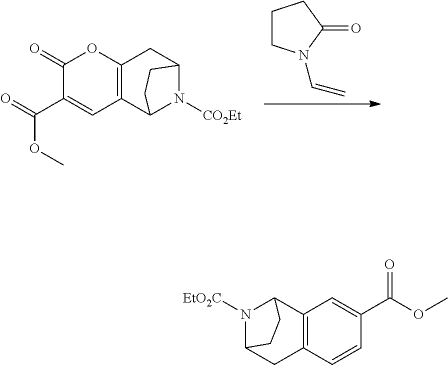

To a suspension of 10-ethyl 3-methyl 2-oxo-2,5,6,7,8,9-hexahydro-5,8-epiminocyclohepta[b]pyran-3,10-dicarboxylate (65.97 g, 215 mmol, 1.0 eq) in 400 mL of mesitylene was added 1-vinylpyrrolidin-2-one (47.7 g, 430 mmol, 2.0 eq, 45.87 mL). The mixture was stirred at 180° C. for 7 h and then at 130° C. for 16 h. It turned into brown solution. TLC (PE:EA=2:1) showed one new spot. The mixture was concentrated under reduced pressure and the residue was diluted with EtOAc (500 mL), washed with 5% HCl in brine (200 mL×3). The organic layer was dried over Na₂SO₄, filtered, concentrated and purified by column chromatography on silica gel (PE:EA=50:1˜10:1) to supply 10-ethyl 3-methyl 6,7,8,9-tetrahydro-5H-5,8-epiminobenzo[7]annulene-3,10-dicarboxylate (42.9 g, 69.2% yield) as white solid. ¹HNMR: (400 MHz, CDCl₃) δ: 7.85-7.69 (m, 2H), 7.14 (m, 1H), 5.03 (br s, 1H), 4.60 (br s, 1H), 4.16-3.97 (m, 2H), 3.92-3.84 (s, 3H), 3.41 (m, 1H), 2.63 (m, 1H), 2.30-2.11(m, 2H), 1.91-1.80 (m, 1H), 1.67 (m, 1H), 1.21 (br s, 3H).

### 4. Preparation of 10-(ethoxycarbonyl)-6,7,8,9-tetrahydro-5H-5,8-epiminobenzo[7]annulene-3-carboxylic acid

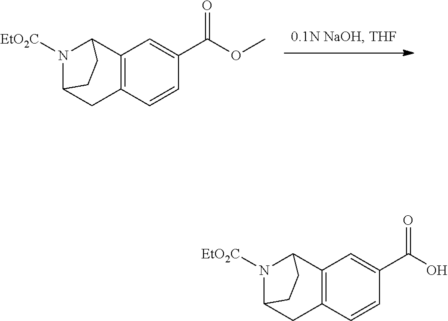 [SMILES: CCOC(=O)N1C2CCC1c1cc(C(=O)OC)ccc1C2 ; CCOC(=O)N1C2CCC1c1cc(C(=O)O)ccc1C2]

To a suspension of 10-ethyl 3-methyl 6,7,8,9-tetrahydro-5H-5,8-epiminobenzo[7]annulene-3,10-dicarboxylate (42.9 g, 148 mmol, 1.0 eq) in 500 mL of THF was added NaOH (296 mL, 296 mmol, 2.0 eq, 1M in water). The suspension was stirred at 18˜20° C. for 2 days. It turned into solution. The mixture was concentrated under reduced pressure to remove THF. The residue was diluted with water (1 L), adjusted pH to 4˜5 by adding 1M HCl aq. then extracted with EtOAc (500 mL×3). The combined organic layers were dried over Na₂SO₄, filtered, concentrated to supply 10-(ethoxycarbonyl)-6,7,8,9-tetrahydro-5H-5,8-epiminobenzo[7]annulene-3-carboxylic acid (39 g, 95.8% yield) as white solid. ¹HNMR: (400 MHz, CDCl₃) δ: 7.93-7.86 (m, 1H), 7.83 (br s, 1H), 7.18 (m, 1H), 5.07 (br s, 1H), 4.65 (br s, 1H), 4.20-4.02 (m, 2H), 3.45 (br s, 1H), 2.66 (d, J=17.2 Hz, 1H),2.34-2.17 (m, 2H), 1.91 (m, 1H), 1.76-1.61 (m, 1H), 1.32-1.14 (m, 3H).

### 5. Preparation of ethyl 3-isocyanato-6,7,8,9-tetrahydro-5H-5,8-epiminobenzo[7]annulene-10-carboxylate

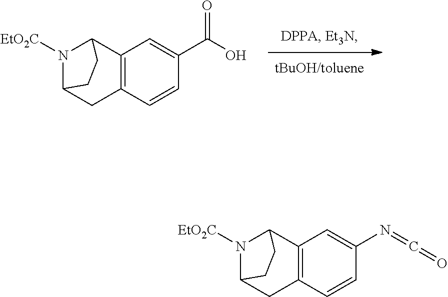 [SMILES: CCOC(=O)N1C2CCC1c1cc(C(=O)O)ccc1C2 ; CCOC(=O)N1C2CCC1c1cc(N=C=O)ccc1C2]

To the solution of 10-(ethoxycarbonyl)-6,7,8,9-tetrahydro-5H-5,8-epiminobenzo[7]annulene-3-carboxylic acid (39 g, 142 mmol, 1.0 eq) and TEA (39.8 mL, 284 mmol, 2.0 eq) in 400 mL of t-BuOH was added DPPA (46.86 g, 170 mmol, 1.2 eq) by dropwise at 10-13° C. After addition, the resulting mixture was stirred at 30° C. for 18 h. TLC (PE:EA=3:1) showed the new spot. The mixture was concentrated directly and purified by by column chromatography on silica gel (PE:EA=25:1-15:1-10:1) to supply ethyl 3-isocyanato-6,7,8,9-tetrahydro-5H-5,8-epiminobenzo[7]annulene-10-carboxylate (37.5 g, 97% yield) as white solid. ¹HNMR: (400 MHz, CDCl₃) δ: 7.81 (dd, J=1.8, 8.2 Hz, 1H), 7.75 (br s, 1H), 7.17 (d, J=8.0 Hz, 1H), 5.04 (br s, 1H), 4.63 (br s, 1H), 4.18-3.98 (m, 2H), 3.43 (br s, 1H), 2.65 (d, J=17.6 Hz, 1H), 2.33-2.16 (m, 2H), 1.88 (m, 1H), 1.73-1.60 (m, 1H), 1.22 (br s, 3H).

### 6. Preparation of ethyl 3-amino-6,7,8,9-tetrahydro-5H-5,8-epiminobenzo[7]annulene-10-carboxylate

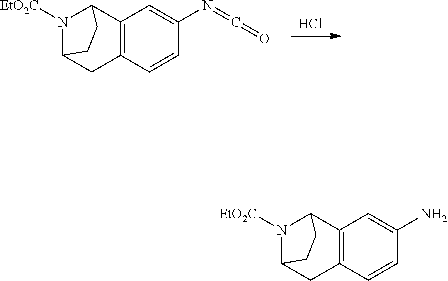 [SMILES: CCOC(=O)N1C2CCC1c1cc(N=C=O)ccc1C2 ; Cl ; CCOC(=O)N1C2CCC1c1cc(N)ccc1C2]

ethyl 3-isocyanato-6,7,8,9-tetrahydro-5H-5,8-epiminobenzo[7]annulene-10-carboxylate (37.5 g, 138 mmol, 1.0 eq) in 300 mL of dioxane was added into 390 mL of HCl (4M in water) at 80° C. by dropwise. After addition, the resulting mixture was stirred at 80° C. for 18 h. The mixture was concentrated under reduced pressure to remove dioxane. The residue was diluted with water (500 mL), washed with MTBE (200 mL×3). The aqueous phase was adjust pH to ˜10 by adding 15% NaOH aq, extracted with EtOAc (300 mL×3), the combined organic layers were washed with brine (200 mL), dried over Na₂SO₄, filtered and concentrated to supply ethyl 3-amino-6,7,8,9-tetrahydro-5H-5,8-epiminobenzo[7]annulene-10-carboxylate (23.35 g, 68.9% yield) as pale-yellow solid. ¹HNMR: (400 MHz, CDCl₃) δ: 6.86 (br d, J=7.9 Hz, 1H), 6.51 (dd, J=2.364, 8.0 Hz, 1H), 6.42 (br s, 1H), 4.84 (br s, 1H), 4.59 (br s, 1H), 4.19-3.96 (m, 2H), 3.55 (br s, 2H), 3.29 (br s, 1H), 2.46 (d, J=16.1 Hz, 1H), 2.26-2.09 (m, 2H), 1.92-1.81 (m, 1H), 1.73-1.55 (m, 1H), 1.29-1.15 (m, 3H).

### 7. Preparation of ethyl 3-bromo-6,7,8,9-tetrahydro-5H-5,8-epiminobenzo[7]annulene-10-carboxylate

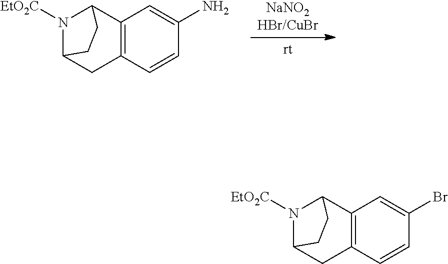 [SMILES: CCOC(=O)N1C2CCC1c1cc(N)ccc1C2 ; "NaNO2HBr/CuBr" ; "rt" ; CCOC(=O)N1C2CCC1c1cc(Br)ccc1C2]

To the solution of ethyl 3-amino-6,7,8,9-tetrahydro-5H-5,8-epiminobenzo[7]annulene-10-carboxylate (23.35 g, 94.92 mmol, 1.0 eq) in HBr (115 mL, 40% in HOAc), water (115 mL) and acetonitrile (40 mL) was added NaNO₂ (7.2 g, 104.40 mmol, 1.1 eq) under ice-bath. The resulting mixture was stirred at for 2 h at 0˜5° C. Compound CuBr (20.50 g, 142.38 mmol, 1.5 eq) in HBr (115 mL, 40% in HOAc) was added by dropwise and the mixture was stirred at 12˜18° C. for 18 h. TLC (PE:EA=3:1) showed one new spot was observed. The mixture was diluted with water (800 mL), extracted with EtOAc (300 mL×3), the combined organic layers were washed with 5% NaOH aq. (200 mL×2), brine (200 mL), dried over Na₂SO₄, filtered, concentrated and purified by column chromatography on silica gel (PE:EA=30:1-20:1-15:1) to supply ethyl 3-bromo-6,7,8,9-tetrahydro-5H-5,8-epiminobenzo[7]annulene-10-carboxylate (18.0 g, 61.2% yield) as colorless gum. ¹HNMR: (400 MHz, CDCl₃) δ: 7.18-7.31 (m, 2H), 6.95 (br d, J=7.9 Hz, 1H), 4.91 (br s, 1H), 4.60 (br s, 1H), 3.97-4.22 (m, 2H), 3.32 (br s, 1H), 2.52 (d, J=17.1 Hz, 1H), 2.13-2.30 (m, 2H), 1.88 (br t, J=9.4 Hz, 1H), 1.64 (br s, 1H), 1.30-1.15 (m, 3H).

### 8. Preparation of 3-bromo-6,7,8,9-tetrahydro-5H-5,8-epiminobenzo[7]annulene

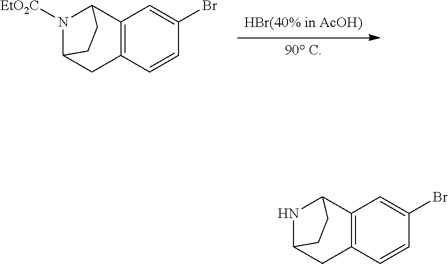 [SMILES: CCOC(=O)N1C2CCC1c1cc(Br)ccc1C2 ; "90° C." ; "HBr(40% in AcOH)" ; Brc1ccc2c(c1)C1CCC(C2)N1]

Ethyl 3-bromo-6,7,8,9-tetrahydro-5H-5,8-epiminobenzo[7]annulene-10-carboxylate (18.0 g, 58.06 mmol, 1.0 eq) in toluene (200 mL) was added into HBr (220 mL, 40% in HOAc). The resulting mixture was stirred at 100° C. for 18 h. The mixture was cooled and became into two phases. TLC (PE:EA=3:1) showed the above layer contained starting material. The two layers were separated, the above layer was concentrated, the residue was dissolved into 150 mL of HBr (40% in HOAc) and stirred at 100° C. for 18 h. TLC (PE:EA=3:1) showed starting material was consumed completely. The reaction mixture combined with the separated layer were concentrated, the residue was diluted with water (300 mL), adjusted pH to 10 by adding 1M NaOH aq, extracted with EtOAc (150 mL×3), the combined organic layers were washed with brine (100 mL), dried over Na₂SO₄, filtered, concentrated. After standing for 18 h at 5˜24° C., the solid formed was collected and washed with MTBE (10 mL) to supply 3-bromo-6,7,8,9-tetrahydro-5H-5,8-epiminobenzo[7]annulene (6.1 g, 44.53% yield) as grey solid. LCMS: (M+H:237.7).

### 9. Preparation of ((5S,8R)-3-bromo-6,7,8,9-tetrahydro-5H-5,8-epiminobenzo[7]annulen-10-yl)(phenyl)methanone and ((5R,8S)-3-bromo-6,7,8,9-tetrahydro-5H-5,8-epiminobenzo[7]annulen-10-yl)(phenyl)methanone

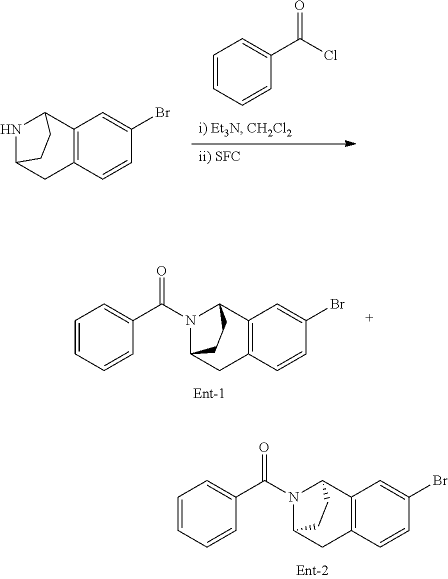

To a mixture of 3-bromo-6,7,8,9-tetrahydro-5H-5,8-epiminobenzo[7]annulene (2.0 g, 8.44 mmol, 1.0 eq) in DCM (30 mL) was added TEA (1.7 g, 2.37 mL, 16.88 mol, 2.0 eq) and benzoyl chloride (1.77 g, 12.66 mmol, 1.5 eq). The resulting mixture was stirred at 13˜18° C. for 18 h. TLC (PE:EA=3:1) showed one new spot was observed. The reaction mixture was concentrated, the residue was diluted with EtOAc (30 mL), washed with brine (20 mL×2), dried over Na₂SO₄, filtered, concentrated and purified by column chromatography on silica gel (PE:EA=20:1-15:1-10:1) to supply (3-bromo-6,7,8,9-tetrahydro-5H-5,8-epiminobenzo[7]annulen-10-yl)(phenyl)methanone (1.1 g, 38.1% yield) as pale-yellow solid which was separated by SFC (Mobile phase: supercritical CO₂/EtOH (0.1% NH₃H₂O); Column: AD 250*30 mm*10 um; Detection wavelength: 220 nm) to give enantiomer 1 as Ent-1 (Rt=2.018 min, 440 mg, 13.9% yield) as white solid and enantiomer 2 as Ent-2 (Rt=2.289 min, 450 mg, 15.6% yield) as white solid. Ent-1: LCMS: (M+H: 342.1). ¹HNMR: (400 MHz, CDCl₃) δ: 7.31-7.50 (m, 6H), 9.68-7.03 (m, 2H), 5.18-5.51 (m, 1H), 4.37-4.74 (m, 1H), 3.18-3.49 (m, 1H), 2.56-2.70 (m, 2H), 2.16-2.25 (m, 2H), 1.91-1.96 (m, 1H), 1.72-1.76 (m, 1H). Ent-2: LCMS: (M+H: 342.1); ¹HNMR: (400 MHz, CDCl₃) δ: 7.31-7.51 (m, 6H), 9.68-7.03 (m, 2H), 5.18-5.51 (m, 1H), 4.37-4.74 (m, 1H), 3.18-3.49 (m, 1H), 2.56-2.70 (m, 2H), 2.16-2.25 (m, 2H), 1.91-1.96 (m, 1H), 1.72-1.76 (m, 1H). The absolute configuration of Ent-1 and Ent-2 were arbitrarily assigned.

### 10. Preparation of phenyl(3-(4,4,5,5-tetramethyl-1,3,2-dioxaborolan-2-yl)-6,7,8,9-tetrahydro-5H-5,8-epiminobenzo[7]annulen-10-yl)methanone (Ent-2-A)

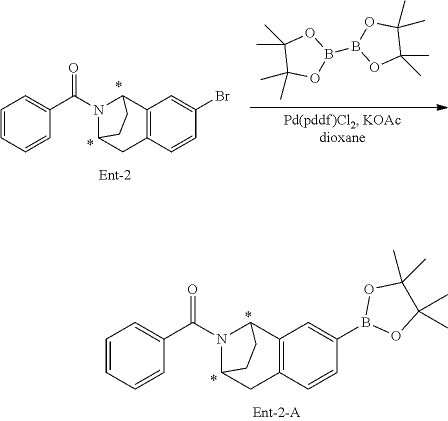

The above isomer Ent-2 (450 mg, 1.32 mmol, 1.0 eq), PinB-BPin (501 mg, 1.97 mmol, 1.5 eq), KOAc (259 mg, 2.64 mmol, 2.0 eq) and Pd(dppf)Cl₂ (96 mg, 0.13 mmol, 0.1 eq) were added into 15 mL of dioxane. The mixture was stirred at 100° C. for 3 h under N₂ atmosphere. TLC (PE:EA=3:1) showed only one spot. The mixture was diluted with EtOAc (30 mL), filtered through a pad of celite. The filtrate was concentrated and purified by prep-TLC (PE:EA=2:1) to supply the corresponding phenyl(3-(4,4,5,5-tetramethyl-1,3,2-dioxaborolan-2-yl)-6,7,8,9-tetrahydro-5H-5,8-epiminobenzo[7]annulen-10-yl)methanone (Ent-2-A) (480 mg, 76.6% yield) as white solid. LCMS: (M+H: 342.1). ¹HNMR: (400 MHz, CDCl₃) δ: 7.64-7.66 (m, 1H), 7.31-7.51 (m, 6H), 7.13-7.17 (m, 1H), 5.18-5.61 (m, 1H), 4.36-4.85 (m, 2H), 2.64-2.76 (m, 1H), 2.16-2.29 (m, 2H), 1.96-2.16 (m, 1H), 1.73-1.74 (m, 1H), 1.25-1.35 (m, 12H).

### 11. Preparation of ethyl (3S)-3-(10-benzoyl-6,7,8,9-tetrahydro-5H-5,8-epiminobenzo[7]annulen-3-yl)-3-(1-ethyl-4-methyl-1H-benzo[d][1,2,3]triazol-5-yl)propanoate (Ent-2-B-1) and ethyl (3R)-3-(10-benzoyl-6,7,8,9-tetrahydro-5H-5,8-epiminobenzo[7]annulen-3-yl)-3-(1-ethyl-4-methyl-1H-benzo[d][1,2,3]triazol-5-yl)propanoate (Ent-2-B-2)

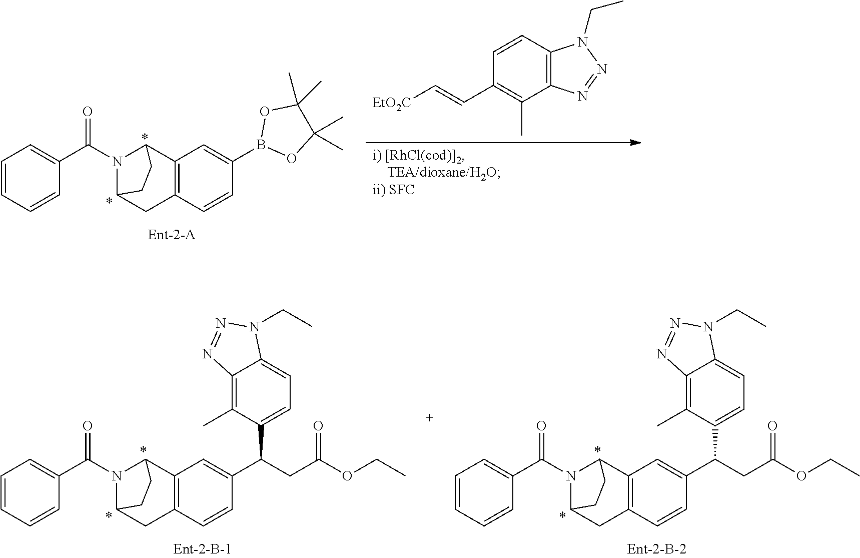

The above compound Ent-2-A (300 mg, 0.77 mmol, 1.0 eq, purity: 85%), ethyl (E)-3-(1-ethyl-4-methyl-1H-benzo[d][1,2,3]triazol-5-yl)acrylate (240 mg, 0.93 mmol, 1.2 eq), TEA (233 mg, 2.3 mmol, 3.0 eq) and compound [RhCl(cod)]₂ (19 mg, 0.04 mmol, 0.05 eq) were added into 12 mL of dioxane/water (v:v=5:1). The reaction mixture was stirred at 130° C. for 18 h under nitrogen atmosphere. The mixture was diluted with EA (20 mL), filtered through a pad of celite. The filtrate was concentrated and the residue was diluted with EA (50 mL), washed with water (20 mL×2), concentrated and purified by prep-TLC (PE:EA=1:1) for three times to supply The racemic compound ethyl 3-(10-benzoyl-6,7,8,9-tetrahydro-5H-5,8-epiminobenzo[7]annulen-3-yl)-3-(1-ethyl-4-methyl-1H-benzo[d][1,2,3]triazol-5-yl)propanoate (Ent-2-B, 110 mg) as pale-yellow solid which was separated by SFC (Mobile phase: supercritical CO₂/MeOH (0.1% NH₃H₂O); Column: AD 250*30 mm*10 um; Detection wavelength: 220 nm) to give enantiomer Ent-2-B-1 (35 mg, 8.7% yield) as white solid and enantiomer Ent-2-B-2 (43 mg, 10.7% yield) as white solid. Ent-2-B-1: LCMS: (M+H: 523.2), Ent-2-B-2: LCMS (M+H: 523.2).

### 12. Preparation of (3S)-3-(10-benzoyl-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-2-C-1)

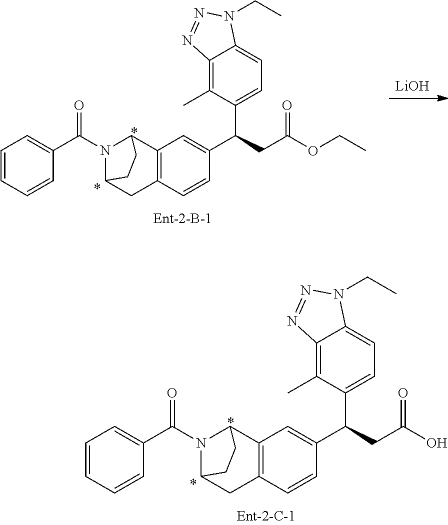 [SMILES: CCOC(=O)C[C@@H](c1ccc2c(c1)C1CCC(C2)N1C(=O)c1ccccc1)c1ccc2c(nnn2CC)c1C ; "LiOH" ; CCn1nnc2c(C)c([C@@H](CC(=O)O)c3ccc4c(c3)C3CCC(C4)N3C(=O)c3ccccc3)ccc21]

The above compound Ent-2-B-1 (35 mg, 0.067 mmol, 1.0 eq) and LiOH.H₂O (28 mg, 0.68 mmol, 10 eq) were added into 6 mL of EtOH/H₂O (V:V=2:1). The mixture was stirred at 7˜17° C. for 18 h. The mixture was concentrated under reduced pressure to remove EtOH, diluted with water (25 mL), and washed with MTBE (10 mL). The aqueous phase was adjusted pH to 1˜2 by adding 1M HCl aq, extracted with EtOAc (10 mL×3). The combined organic layers were washed with water (10 mL), dried over anhydrous Na₂SO₄, filtered, concentrated under reduced pressure to supply crude compound Ent-2-C-1 (30 mg, 90.9% yield) as pale-yellow solid. LCMS: (M+Na: 495.2). ¹HNMR: (400 MHz, CDCl₃) δ7.27-7.48 (m, 7H), 6.31-7.25(m, 3H), 4.93-5.78 (m, 1H), 4.91-4.93 (m, 1H), 4.62-4.67 (m, 2H), 4.62-4.31 (m, 1H), 3.06-3.48 (m, 3H), 2.80-2.83 (m, 3H), 2.63-2.67 (m, 1H), 2.05-2.20 (m, 2H), 1.84-1.99(m, 1H), 1.68-1.84 (m, 1H), 1.60 (t,J=7.2 Hz, 3H).

### 13. Preparation of (3R)-3-(10-benzoyl-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-2-C-2)

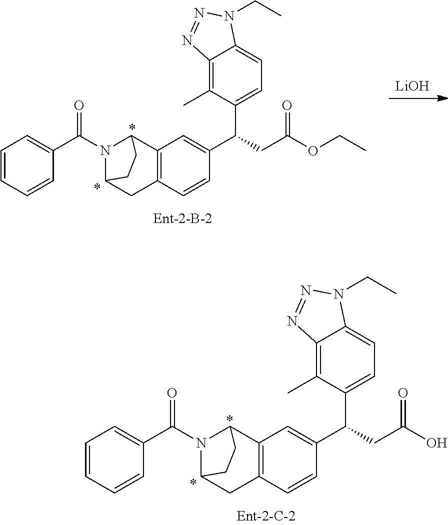 [SMILES: CCOC(=O)C[C@H](c1ccc2c(c1)C1CCC(C2)N1C(=O)c1ccccc1)c1ccc2c(nnn2CC)c1C ; "LiOH" ; CCn1nnc2c(C)c([C@H](CC(=O)O)c3ccc4c(c3)C3CCC(C4)N3C(=O)c3ccccc3)ccc21]

The above compound Ent-2-B-2 (43 mg, 0.082 mmol, 1.0 eq) and LiOH.H₂O (34 mg, 0.82 mmol, 10 eq) were added into 6 mL of EtOH/H₂O (V:V=2:1). The mixture was stirred at 7˜17° C. for 18 h. The mixture was concentrated under reduced pressure to remove EtOH, diluted with water (15 mL), adjusted pH to 1˜2 by adding 1M HCl aq, extracted with EtOAc (10 mL×3). The combined organic layers were washed with water (10 mL), dried over anhydrous Na₂SO₄, filtered, concentrated under reduced pressure to supply crude compound Ent-2-C-2 (18 mg, 93.8% yield) as pale-yellow solid. LCMS: (M+H: 495.2). ¹HNMR: (400 MHz, CDCl₃) δ: 7.22-7.47 (m, 7H), 6.94-7.08(m, 2H), 6.42-6.94 (m, 1H), 5.14-5.72 (m, 1H), 4.94-4.97 (m, 1H), 4.61-4.66 (m, 2H), 4.61-4.30 (m, 1H), 3.06-3.43 (m, 3H), 2.83 (s, 3H), 2.63-2.67 (m, 1H), 1.99-2.14 (m, 2H), 1.84-1.89(m, 1H), 1.62-1.84 (m, 1H), 1.58 (t, J=6.8 Hz,3H).

### Examples 155 and 156

### (3S)-3-(10-(3,4-dimethylbenzoyl)-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-2-D-1) and (3R)-3-(10-(3,4-dimethylbenzoyl)-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-2-D-2)

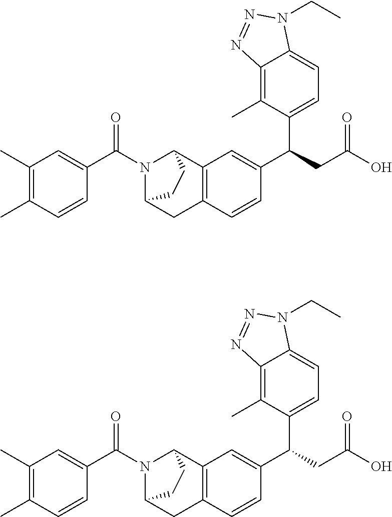 [SMILES: CCn1nnc2c(C)c([C@@H](CC(=O)O)c3ccc4c(c3)[C@H]3CC[C@@H](C4)N3C(=O)c3ccc(C)c(C)c3)ccc21 ; CCn1nnc2c(C)c([C@H](CC(=O)O)c3ccc4c(c3)[C@H]3CC[C@@H](C4)N3C(=O)c3ccc(C)c(C)c3)ccc21]

### 1. Preparation of compound 13-1 and 13-2

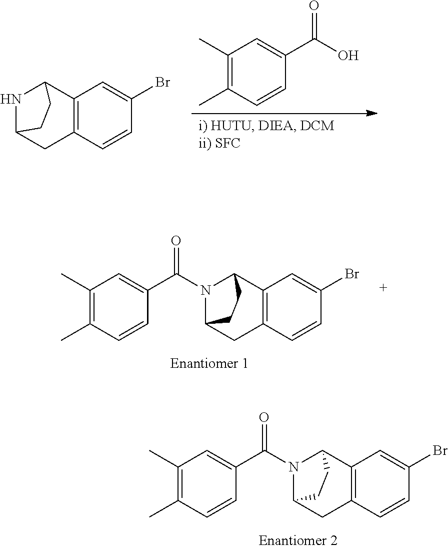

3,4-Dimethylbenzoic acid (760 mg, 5.1 mmol, 1.2 eq,), HATU (1.94 g, 5.1 mmol, 1.2 eq) and DIEA (1.08 g, 8.4 mmol, 2.0 eq) were added into 20 mL of DCM and stirred at 10˜20° C. for 30 min. It turned into solution for suspension. 3-Bromo-6,7,8,9-tetrahydro-5H-5,8-epiminobenzo[7]annulene (1.0 g, 4.2 mmol, 1.0 eq) was added and the resulting mixture was stirred at 10˜20° C. for 18 h. The mixture was concentrated and the residue was diluted with water (40 mL), extracted with EtOAc (25 mL×3). The combined organic layers were dried over Na₂SO₄, filtered, concentrated and purified by flash column chromatography (PE:EA=20:1-10:1) to supply (3-bromo-6,7,8,9-tetrahydro-5H-5,8-epiminobenzo[7]annulen-10-yl)(3,4-dimethylphenyl)methanone (1.1 g) as white solid which was separated by SFC. (Mobile phase: supercritical CO₂/EtOH (0.1% NH₃H₂O); Column: C2 250*30 mm*10 um; Detection wavelength: 220 nm) to give enantiomer 1 (Rt=5.543 min, 500 mg, 32.2% yield) as pale-yellow solid and enantiomer 2 (Rt=6.363 min, 460 mg, 29.6% yield) as pale-yellow solid.

### 2. Preparation of (3S)-3-(10-(3,4-dimethylbenzoyl)-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-2-D-1) and (3R)-3-(10-(3,4-dimethylbenzoyl)-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-2-D-2)

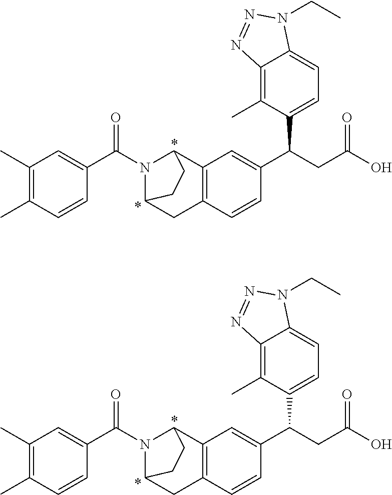 [SMILES: CCn1nnc2c(C)c([C@@H](CC(=O)O)c3ccc4c(c3)C3CCC(C4)N3C(=O)c3ccc(C)c(C)c3)ccc21 ; CCn1nnc2c(C)c([C@H](CC(=O)O)c3ccc4c(c3)C3CCC(C4)N3C(=O)c3ccc(C)c(C)c3)ccc21]

Starting from the above enantiomer 2 and following the procedures of Example 152, Ent-2-D-1 and Ent-2-D-2 were made. Compound Ent-2-D-1 (11 mg, 58.5% yield) as white solid. ¹HNMR: (400 MHz, CDCl₃) δ: 7.20-7.37 (m, 2H), 6.70-7.15(m, 6H), 5.15-5.52 (m, 1H),4.94-4.95 (m, 1H),4.63-4.70 (m, 2H), 4.37-4.70 (m, 1H),3.23-3.44 (m, 3H), 2.85 (s, 3H), 2.60-2.67 (m, 1H), 2.09-2.28 (m, 8H), 1.80-1.82 (m, 1H), 1.62-1.67 (m, 1H), 1.60 (t, J=7.2 Hz, 3H). LCMS: (M+H: 523.2). Compound Ent-2-D-2 (17 mg, 74.2% yield) as white solid. ¹HNMR: (400 MHz, CDCl₃) δ: 7.31-7.34 (m, 2H), 6.76-7.09 (m, 6H), 5.15-5.47 (m, 1H),4.95-4.97 (m, 1H),4.62-4.69 (m, 2H), 4.36-4.70 (m, 1H), 3.23-3.47 (m, 3H), 2.81 (s, 3H), 2.60-2.68 (m, 1H), 2.09-2.28 (m, 8H), 1.82-1.84 (m, 1H), 1.65-1.67 (m, 1H), 1.61 (t, J=6.8 Hz, 3H). LCMS: (M+H: 523.2).

### Examples 157, 158 and 159

### (S)-3-((5S,8R)-10-(2,5-dimethylbenzoyl)-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-1-E-1), (R)-3-((5S,8R)-10-(2,5-dimethylbenzoyl)-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-1-E-2) and (S)-3-((5R,8S)-10-(2,5-dimethylbenzoyl)-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-2-E-3)

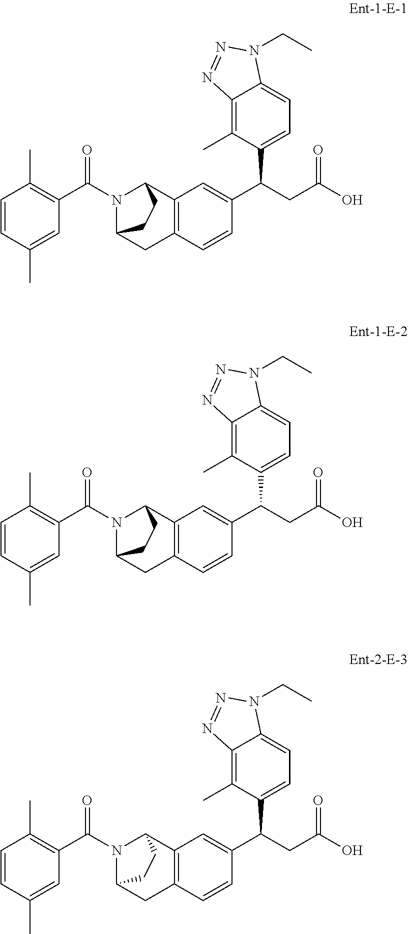 [SMILES: CCn1nnc2c(C)c([C@@H](CC(=O)O)c3ccc4c(c3)[C@@H]3CC[C@H](C4)N3C(=O)c3cc(C)ccc3C)ccc21 ; CCn1nnc2c(C)c([C@H](CC(=O)O)c3ccc4c(c3)[C@@H]3CC[C@H](C4)N3C(=O)c3cc(C)ccc3C)ccc21 ; CCn1nnc2c(C)c([C@@H](CC(=O)O)c3ccc4c(c3)[C@H]3CC[C@@H](C4)N3C(=O)c3cc(C)ccc3C)ccc21]

### 1. Preparation of ((5S,8R)-3-bromo-6,7,8,9-tetrahydro-5H-5,8-epiminobenzo[7]annulen-10-yl)(2,5-dimethylphenyl)methanone and ((5R,8S)-3-bromo-6,7,8,9-tetrahydro-5H-5,8-epiminobenzo[7]annulen-10-yl)(2,5-dimethylphenyl)methanone

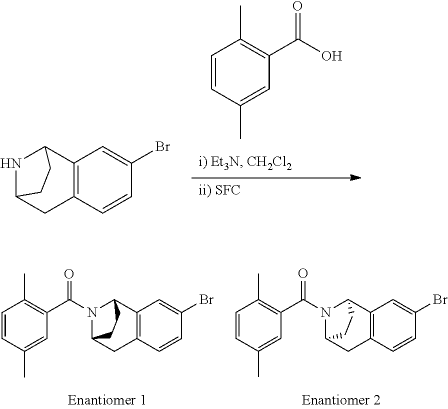

To a mixture of 3-bromo-6,7,8,9-tetrahydro-5H-5,8-epiminobenzo[7]annulene (1.0 g, 0.0042 mol, 1.0 eq) and 2,5-dimethylbenzoic acid (0.945 g, 0.0063 mol, 1.5 eq) and HATU (2.394 g, 0.0063 mol, 1.5 eq) in DCM (30 mL) was added TEA (1.7 g, 0.0168 mol, 4.0 eq). The mixture was stirred at 10˜15° C. for 15 h. TLC (PE:EA=3:1) showed the starting material was almost consumed and a new spot was observed. The mixture was concentrated to give the residue, which was diluted with H₂0 (10 mL) and extracted with DCM (10 mL×3). The combined organic layer was dried over Na₂SO₄ and filtered. The filtrate was concentrated to give the residue, which was purified by prep-TLC (PE:EA=2:1) to supply (3-bromo-6,7,8,9-tetrahydro-5H-5,8-epiminobenzo[7]annulen-10-yl)(2,5-dimethylphenyl)methanone (800 mg, 51% yield) as solid. ¹HNMR (400 MHz, CDCl₃) δ=6.87-7.36 (m, 6H), 5.10-5.10 (m, 1H), 4.03-4.40 (m, 1H), 3.04-3.58 (m, 1H), 2.51-2.66 (m, 1H), 2.21-2.36 (m, 6H), 2.10-2.19 (m, 1H), 1.96-2.06 (m, 1H), 1.90 (mt, 1H), 1.64-1.79 (m, 1H).

The racemate was separated by SFC (Column: Lux Cellulose-2 150×4.6 mm I.D., 3 um, Mobile phase: A: CO2 B: Ethanol (0.05% DEA), Gradient: from 5% to 40% of B in 5.5 min and hold 40% for 3 min, then 5% of B for 1.5 min, Flow rate: 2.5 mL/min Column temperature: 40 C; Detection wavelength: 220 nm) to give enantiomer 1 (Rt=4.587 min, 300 mg, 37.5% yield, ee %: 96.7%) as white solid and enantiomer 2 (Rt=4.972 min, 300 mg, 37.5% yield, ee %: 99.3%) as white solid.

### 2. (S)-3-((5S,8R)-10-(2,5-dimethylbenzoyl)-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-1-E-1) and (R)-3-((5S,8R)-10-(2,5-dimethylbenzoyl)-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-1-E-2)

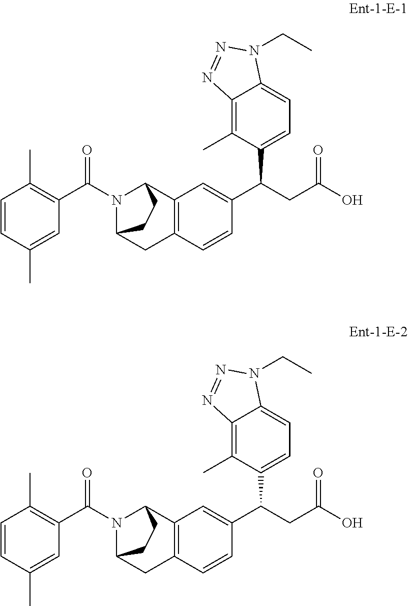 [SMILES: CCn1nnc2c(C)c([C@@H](CC(=O)O)c3ccc4c(c3)[C@@H]3CC[C@H](C4)N3C(=O)c3cc(C)ccc3C)ccc21 ; CCn1nnc2c(C)c([C@H](CC(=O)O)c3ccc4c(c3)[C@@H]3CC[C@H](C4)N3C(=O)c3cc(C)ccc3C)ccc21]

Starting from the above enantiomer 1 and following the procedures of Example 152, Ent-1-E-1 and Ent-1-E-2 were made. Ent-2-E-1 (15 mg, yield: 16%) as a white solid. ¹HNMR: (400 MHz, CDCl₃) δ=7.39-7.58 (m, 1H), 7.34 (s, 1H), 6.47-7.23 (m, 6H), 5.12-5.57 (m, 1H), 4.84-5.03 (m, 1H), 4.70 (s, 2H), 4.00-4.31 (m, 1H), 2.96-3.54 (m, 3H), 2.92-2.80 (m, 3H), 2.51-2.71 (m, 1H), 2.14-2.42 (m, 6H), 1.68-2.11 (m, 4H), 1.65 (m, 3H). LCMS: (M+H: 523.3). Ent-2-E-2 (57 mg, yield: 83%) as a white solid. ¹HNMR: 19395-56-1C (400 MHz, CDCl₃) δ=7.39-7.58 (m, 1H), 7.34 (s, 1H), 6.47-7.23 (m, 6H), 5.12-5.57 (m, 1H), 4.84-5.03 (m, 1H), 4.70 (s, 2H), 4.00-4.31 (m, 1H), 2.96-3.54 (m, 3H), 2.92-2.80 (m, 3H), 2.51-2.71 (m, 1H), 2.14-2.42 (m, 6H), 1.68-2.11 (m, 4H), 1.65 (m, 3H). LCMS: (M+H: 523.3).

### 3. (S)-3-((5R,8S)-10-(2,5-dimethylbenzoyl)-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-2-E-3) and (R)-3-((5R,8S)-10-(2,5-dimethylbenzoyl)-6,7,8,9-tetrahydro-5H-5,8-epiminobenzo[7]annulen-3-yl)-3-(1-ethyl-4-methyl-1H-benzo[d][1,2,3]triazol-5-yl)propanoic acid (Ent-2-E-4)

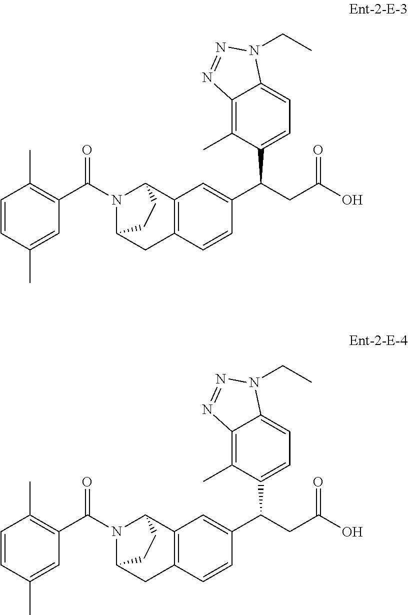 [SMILES: CCn1nnc2c(C)c([C@@H](CC(=O)O)c3ccc4c(c3)[C@H]3CC[C@@H](C4)N3C(=O)c3cc(C)ccc3C)ccc21 ; CCn1nnc2c(C)c([C@H](CC(=O)O)c3ccc4c(c3)[C@H]3CC[C@@H](C4)N3C(=O)c3cc(C)ccc3C)ccc21]

Starting from the above enantiomer 2 and following the procedures of Example 152, Ent-2-E-3 and Ent-2-E-4 were made. Ent-2-E-3 (54 mg, yield: 71%) as a white solid. ¹HNMR (400 MHz, CDCl₃) δ=7.39-7.58 (m, 1H), 7.34 (s, 1H), 6.47-7.23 (m, 6H), 5.12-5.57 (m, 1H), 4.84-5.03 (m, 1H), 4.70 (s, 2H), 4.00-4.31 (m, 1H), 2.96-3.54 (m, 3H), 2.92-2.80 (m, 3H), 2.51-2.71 (m, 1H), 2.14-2.42 (m, 6H), 1.68-2.11 (m, 4H), 1.65 (m, 3H). LCMS: (M+H: 523.3). Ent-2-E-4 (57 mg, yield: 83%) as a white solid. ¹HNMR(400 MHz, CDCl₃) δ=7.39-7.58 (m, 1H), 7.34 (s, 1H), 6.47-7.23 (m, 6H), 5.12-5.57 (m, 1H), 4.84-5.03 (m, 1H), 4.70 (s, 2H), 4.00-4.31 (m, 1H), 2.96-3.54 (m, 3H), 2.92-2.80 (m, 3H), 2.51-2.71 (m, 1H), 2.14-2.42 (m, 6H), 1.68-2.11 (m, 4H), 1.65 (m, 3H). LCMS: (M+H: 523.3).

### Examples 160, 161, 162

### (S)-3-(1-ethyl-4-methyl-1H-benzo[d][1,2,3]triazol-5-yl)-3-((5R,8S)-10-(2-fluoro-5-methylbenzoyl)-6,7,8,9-tetrahydro-5H-5,8-epiminobenzo[7]annulen-3-yl)propanoic acid (Ent-2-F-1), (R)-3-(1-ethyl-4-methyl-1H-benzo[d][1,2,3]triazol-5-yl)-3-((5R,8S)-10-(2-fluoro-5-methylbenzoyl)-6,7,8,9-tetrahydro-5H-5,8-epiminobenzo[7]annulen-3-yl)propanoic acid (Ent-2-F-2) and (S)-3-(1-ethyl-4-methyl-1H-benzo[d][1,2,3]triazol-5-yl)-3-((5S,8R)-10-(2-fluoro-5-methylbenzoyl)-6,7,8,9-tetrahydro-5H-5,8-epiminobenzo[7]annulen-3-yl)propanoic acid (Ent-1-F-3)

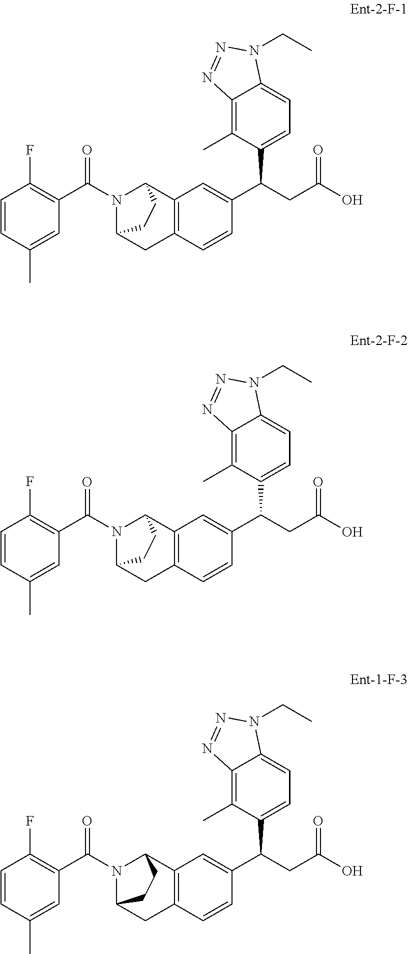 [SMILES: CCn1nnc2c(C)c([C@@H](CC(=O)O)c3ccc4c(c3)[C@H]3CC[C@@H](C4)N3C(=O)c3cc(C)ccc3F)ccc21 ; CCn1nnc2c(C)c([C@H](CC(=O)O)c3ccc4c(c3)[C@H]3CC[C@@H](C4)N3C(=O)c3cc(C)ccc3F)ccc21 ; CCn1nnc2c(C)c([C@@H](CC(=O)O)c3ccc4c(c3)[C@@H]3CC[C@H](C4)N3C(=O)c3cc(C)ccc3F)ccc21]

### 1. Preparation of ((5S,8R)-3-bromo-6,7,8,9-tetrahydro-5H-5,8-epiminobenzo[7]annulen-10-yl)(2-fluoro-5-methylphenyl)methanone and ((5R,8S)-3-bromo-6,7,8,9-tetrahydro-5H-5,8-epiminobenzo[7]annulen-10-yl)(2-fluoro-5-methylphenyl)methanone

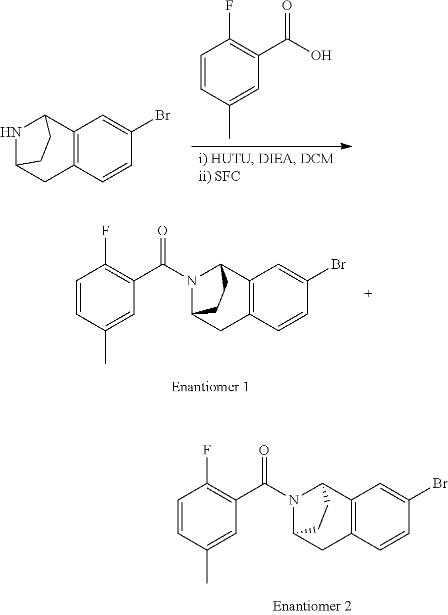

2-Fluoro-5-methylbenzoic acid (780 mg, 5.1 mmol, 1.2 eq), HATU (1.94 g, 5.1 mmol, 1.2 eq) and DIEA (1.08 g, 8.4 mmol, 2.0 eq) were added into 20 mL of DCM and stirred at 10˜20° C. for 40 min. It turned into red solution for suspension 3-bromo-6,7,8,9-tetrahydro-5H-5,8-epiminobenzo[7]annulene (1.0 g, 4.2 mmol, 1.0 eq) was added and the resulting mixture was stirred at 10˜20° C. for 18 h. TLC (PE:EA=2:1) showed one new spot was observed. The mixture was concentrated and the residue was diluted with water (40 mL), extracted with EtOAc (25 mL×3). The combined organic layers were dried over Na₂SO₄, filtered, concentrated and purified by flash column chromatography (PE:EA=20:1-10:1) to supply impure (3-bromo-6,7,8,9-tetrahydro-5H-5,8-epiminobenzo[7]annulen-10-yl)(2-fluoro-5-methylphenyl)methanone (1.5 g) as pale-yellow gum which was purified by prep-HPLC (condition: water (0.025% FA)-CAN, column: Agela ASB 150*25 mm*5 um; Detection wavelength: 220 nm) and then separated by SFC (Mobile phase: supercritical CO₂/EtOH (0.1% NH₃H₂O); Column: C2 250*30 mm*10 um; Detection wavelength: 220 nm) to give enantiomer 1 (Rt=4.530 min, 354 mg, 22.5% yield) as pale-yellow solid and enantiomer 2 (Rt=4.835 min, 385 mg, 24.5% yield) as pale-yellow solid. Enantiomer 1, LCMS: (M+H: 376.0). Enantiomer 2, LCMS: (M+H: 376.0).

### 2. Preparation of (S)-3-(1-ethyl-4-methyl-1H-benzo[d][1,2,3]triazol-5-yl)-3-((5R,8S)-10-(2-fluoro-5-methylbenzoyl)-6,7,8,9-tetrahydro-5H-5,8-epiminobenzo[7]annulen-3-yl)propanoic acid (Ent-2-F-1) and (R)-3-(1-ethyl-4-methyl-1H-benzo[d][1,2,3]triazol-5-yl)-3-((5R,8S)-10-(2-fluoro-5-methylbenzoyl)-6,7,8,9-tetrahydro-5H-5,8-epiminobenzo[7]annulen-3-yl)propanoic acid (Ent-2-F-2)

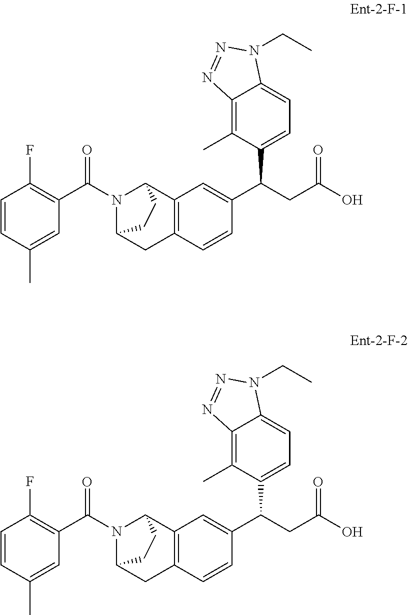 [SMILES: CCn1nnc2c(C)c([C@@H](CC(=O)O)c3ccc4c(c3)[C@H]3CC[C@@H](C4)N3C(=O)c3cc(C)ccc3F)ccc21 ; CCn1nnc2c(C)c([C@H](CC(=O)O)c3ccc4c(c3)[C@H]3CC[C@@H](C4)N3C(=O)c3cc(C)ccc3F)ccc21]

Starting from the above enantiomer 2 and following the procedures of Example 152, Ent-2-F-1 and Ent-2-F-2 were made. Ent-2-F-1 (6 mg, 42.3% yield) as white solid. ¹HNMR (400 MHz, CDCl₃) δ: 7.13-7.42 (m, 3H), 6.61-7.07 (m, 5H), 5.13-5.58 (m, 1H), 4.86-4.98 (m, 1H), 4.65-4.71 (m, 2H), 4.17-4.50 (m, 1H), 2.94-3.52 (m, 3H), 2.74 (d, J=17.2 Hz, 3H), 2.53-2.68 (m, 1H), 2.17-2.31 (m, 5H), 1.79-1.95 (m, 1H), 1.67-1.74 (m, 1H), 1.61-1.64 (m, 3H). LCMS: (M+H: 527.2). Ent-2-F-2 (10 mg, 70% yield) as white solid. ¹HNMR: (400 MHz, CDCl₃) δ: 7.27-7.35 (m, 2H), 6.66-7.18(m, 6H), 5.10-5.48 (m, 1H), 4.88-4.96 (m, 1H), 4.58-4.73 (m, 2H), 4.15-4.47 (m, 1H), 3.06-3.51 (m, 3H), 2.82 (d, J=20.8 Hz, 3H), 2.53-2.67 (m, 1H), 2.15-2.30 (m, 5H), 1.86-1.88 (m, 1H), 1.66-1.75 (m, 1H), 1.58-1.63 (m, 3H). LCMS: (M+H: 527.2).

### 3. Preparation of (S)-3-(1-ethyl-4-methyl-1H-benzo[d][1,2,3]triazol-5-yl)-3-((5S,8R)-10-(2-fluoro-5-methylbenzoyl)-6,7,8,9-tetrahydro-5H-5,8-epiminobenzo[7]annulen-3-yl)propanoic acid (Ent-1-F-3) and (R)-3-(1-ethyl-4-methyl-1H-benzo[d][1,2,3]triazol-5-yl)-3-((5S,8R)-10-(2-fluoro-5-methylbenzoyl)-6,7,8,9-tetrahydro-5H-5,8-epiminobenzo[7]annulen-3-yl)propanoic acid (Ent-1-F-4)

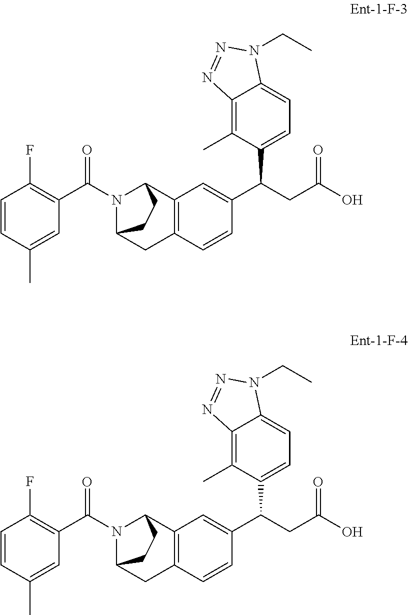 [SMILES: CCn1nnc2c(C)c([C@@H](CC(=O)O)c3ccc4c(c3)[C@@H]3CC[C@H](C4)N3C(=O)c3cc(C)ccc3F)ccc21 ; CCn1nnc2c(C)c([C@H](CC(=O)O)c3ccc4c(c3)[C@@H]3CC[C@H](C4)N3C(=O)c3cc(C)ccc3F)ccc21]

Starting from the above enantiomer 1 and following the procedures of Example 152, Ent-1-F-3 and Ent-1-F-4 were made. Ent-1-F-3 (42 mg, 89.4% yield) as white solid. ¹HNMR: (400 MHz, CDCl₃) δ: 7.25-7.38 (m, 2H), 6.66-7.21(m, 6H), 5.10-5.47 (m, 1H), 4.86-4.97 (m, 1H), 4.61-4.68 (m, 2H), 4.15-4.46 (m, 1H), 2.98-3.50 (m, 3H), 2.84 (d, J=20.8 Hz, 3H), 2.53-2.66 (m, 1H), 2.05-2.30 (m, 5H), 1.83-1.90 (m, 1H), 1.66-1.75 (m, 1H), 1.58-1.61 m, 3H). LCMS: (M+H: 527.2). Ent-1-F-4 (31 mg, 82% yield) as white solid. ¹HNMR (400 MHz, CDCl₃) δ: 7.23-7.39 (m, 2H), 6.64-7.17 (m, 6H), 5.09-5.54 (m, 1H), 4.87-4.98 (m, 1H), 4.61-4.68 (m, 2H), 4.16-4.47 (m, 1H), 3.01-3.52 (m, 3H), 2.80 (d, J=18.4 Hz, 3H), 2.51-2.67 (m, 1H), 2.19-2.31 (m, 5H), 1.79-1.93 (m, 1H), 1.62-1.74 (m, 1H), 1.58-1.61 (m, 3H). LCMS: (M+H: 527.2).

### Example 163

### Cellular Assay

The assay was performed by DiscoverX Corporation, 42501 Albrae Street, Suite 100, Fremont, Calif. 94538. The PathHunter® Nuclear Translocation assay detects translocation of a target protein to, or from, the nucleus. In this system, ProLink™ (PK), a small enzyme fragment, is fused to the protein of interest and EA is localized in the nucleus. Activation of the signaling pathway induces the target protein to either transit into the nucleus, thus forcing complementation of the PK and EA fragments, or out of the nucleus, hindering complementation of the fragments.

EC₅₀ determinations were performed in duplicate at 10 concentrations with 3-fold serial dilutions at a 30 μM top concentration or an otherwise specified top concentration.

Cell handling. PathHunter Pathway cell lines were expanded from freezer stocks according to standard procedures. 5000 cells were seeded in Cell Plating Reagent 0 (containing 1% FBS) to a total volume of 20 uL into white walled, 384-well microplates and incubated for the overnight prior to testing.

Agonist format: For Agonist determination, cells were incubated with sample to induce response. Sample stocks were serially diluted in DMSO to generate 100× sample. Intermediate dilution of sample stocks was performed to generate 5× sample in assay buffer (Cell Plating Reagent 0 containing 1% FBS). 5 μL of 5× sample was added to cells and incubated at room temperature for 6 hours. Vehicle concentration was 1%.

Signal detection: Assay signal was generated through a single addition of 25 μL (100% v/v) of PathHunter Flash Detection reagent, followed by a one hour incubation at room temperature. Microplates were read following signal generation with a PerkinElmer Envision™ instrument for chemiluminescent signal detection.

Data analysis: Compound activity was analyzed using CBIS data analysis suite (Chemlnnovation, Calif.). For agonist mode assays, percentage activity was calculated using the following formula: % Activity=100%×(mean RLU of test sample−mean RLU of vehicle control)/(mean MAX RLU control ligand−mean RLU of vehicle control). For EC50 determination, data was normalized to the maximal and minimal response observed in the presence of the control ligand and vehicle respectively. CDDO methyl ester was used as a control compound.

The compounds described herein were tested for in the above nuclear translocation assay. The results are provided below, wherein the compound number corresponds to the numbers set forth in the examples above, a “+” represents an EC₅₀ of greater than 10 μM, a “++” represents an EC₅₀ of less than or equal to 10 μM, a “+++” represents an EC₅₀ of less than or equal to 1 μM and a “++++” represents an EC₅₀ of less than or equal to 0.1 μM.

| | EC₅₀ (NRF2 | |
|---|---|---|
| COMPOUNDS | TRANSLOCATION) | |
| 133 | xxxx | (<0.1 μM) |
| 9, 11, 13, 26, 27, 28, 32, 47-Ent1, 51, 53, 56, | xxx | (<1 μM) |
| 57-Ent2, 66-Ent1, 68, 69, 70-Ent1, 70-Ent2, | | |
| 78, 80, 83, 84, 85, 97, 99, 100, 102, 103, 104, | | |
| 105, 106, 107, 109, 110, 111, 112, 113, 114, | | |
| 116, 117, 123, 128, 134, 160 and 162 | | |
| 1, 2, 4, 5, 6, 7, 8, 10, 12-Ent1, 12-Ent2, 25, | xx | (<10 μM) |
| 29, 30, 31-Ent1, 31-Ent2, 34, 35, 36, 37, 39, | | |
| 40, 41-Ent2, 43, 47-Ent2, 48-Ent1, 49-Ent1, | | |
| 49-Ent2, 54, 55-Isomer1, 55-Isomer2, 57- | | |
| Ent1, 58-Ent1, 66-Ent2, 73, 79, 86, 87, 88, | | |
| 92, 93, 94, 95, 96, 101, 108, 115, 118, 119, | | |
| 120, 121, 122, 125, 126, 127, 129, 132, 135, | | |
| 136, 138, 140, 141, 154, 155, 156 and 159, | | |
| 3, 14, 15, 16, 17, 18, 19, 20, 21, 22, 23, 24, | x | (>10 μM) |
| 33, 38, 41-Ent1, 42, 44, 45, 46, 48-Ent2, 50, | | |
| 52-Ent1, 52-Ent2, 58-Ent2, 59, 60, 61, 64- | | |
| Ent1, 64-Ent2, 65-Ent1, 65-Ent2, 67, 71, 72- | | |
| isomer 374, 75, 76, 77, 81, 82, 89, 90, 91, 98, | | |
| 137, 139, 142, 143, 144, 145, 146, 147, 148, | | |
| 149, 151, 152, 153, 157, 158 and 161 | | |

### Example 164

### Testing Nrf2 Activator Compounds in Cultured Human Astrocytes

### Cells

Human astrocytes from ScienCell (cat #1820) were grown in astrocyte medium per supplier's instructions. Cells cultured for no more than two passages were plated in 96-well plates at 40,000 cells per well for gene transcription experiments and 20,000 cell per well for glutathione and cytoprotection assays.

### Gene Expression

Primary cultures of human spinal cord astrocytes were treated with compound for 20 hours. The cells were then rinsed in PBS, lysed, and processed for RNA using Ambion Taqman™ Cells-to-CT kit. The resulting cDNA was stored at −20° C. until analysis by real-time polymerase chain reaction (RT-PCR). The cDNA mixture from Cells-to-CT was diluted 5× before loading into PCR. This yields results similar to using 6 ng of purified cDNA. RT-PCR was performed on Life Technologies QuantStudio platform using OpenArray technique according to manufacturer's protocol using the following Taqman primers:

| Target | Taqman assay | |
|---|---|---|
| GCLC | Hs00155249_m1 | glutamate-cysteine ligase, catalytic subunit |
| GCLM | Hs00157694_m1 | glutamate-cysteine ligase, modifier subunit |
| OSGIN1 | Hs00203539_m1 | oxidative stress induced growth inhibitor 1 |
| TBP | Hs00427620_m1 | TATA box binding protein [Homo sapiens |
| | | (human)] |
| PRDX1 | Hs00602020_mH | peroxiredoxin 6 |
| SRXN1 | Hs00607800_m1 | sulfiredoxin 1 |
| TXNRD1 | Hs00917067_m1 | thioredoxin reductase 1 |
| ACTB | Hs01060665_g1 | actin, beta [Homo sapiens(human)] |
| HMOX1 | Hs01110250_m1 | heme oxygenase 1 [Homo |
| | | sapiens(human)] |
| UBC | Hs01871556_s1 | ubiquitin C [Homo sapiens (human)] |
| NQO1 | Hs02512143_s1 | NAD(P)H dehydrogenase, quinone 1 |
| GAPDH | Hs02758991_g1 | glyceraldehyde-3-phosphate |
| | | dehydrogenase [Homo sapiens (human)] |

The comparative CT method was used to calculate fold changes using ThermoFisher Cloud software for PCR analysis. Samples were compared to vehicle control.

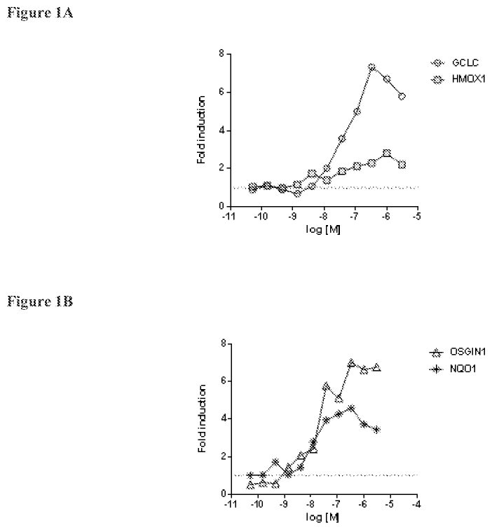

As shown in FIGS. 1A to 1D, Compound 47-Ent1 induces transcription of Nrf2 target genes, including GCLC, HMOX1, OSGIN1 and NQO1.

### Glutathione Assay

Intracellular glutathione was measured after a 20-hr exposure to test compounds by a two-step process. First, cells were lysed and luciferin quantitatively generated from substrate, catalyzed by glutathione-S-transferase in the presence of analyte glutathione. Then luciferin was assayed using stabilized luciferase to produce a luminescent signal proportional to the concentration of glutathione (Promega GSH-Glo, cat #V6912).

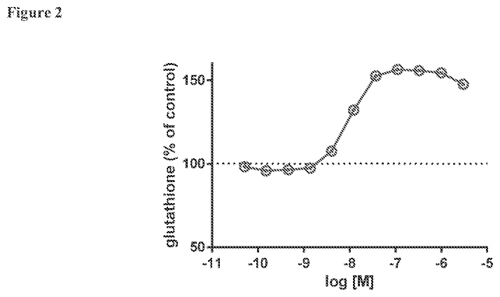

As shown in FIG. 2, Compound 47-Ent1 increases intracellular glutathione.

### Cytoprotection

Astrocytes were treated for 20 hrs as above, then the medium was removed and replaced with serum- and supplement-free growth medium with and without 25 μM sodium arsenite. After 22 hrs., cells were washed with PBS, fixed with 4% paraformaldehyde/4% sucrose in PBS, stained with 4′,6-Diamidino-2-phenylindole dihydrochloride (DAPI) and counted by quantitative fluorescence microscopy.

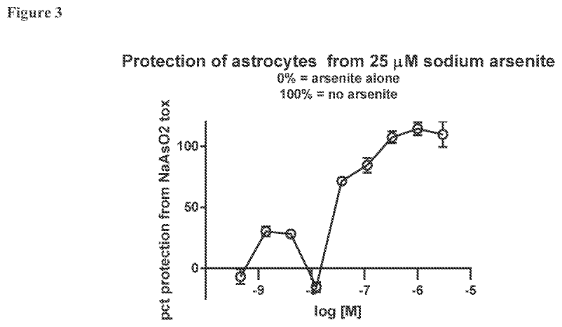

As shown in FIG. 3, Compound 47-Ent1 protects cells from oxidative stress-induced cell death caused by 25 μM sodium arsenite.

### Example 165

### Method for Testing Nrf2 Activator Compounds in Mice

### Animals

Female 6-10 week-old wild type C57BL/6 mice were maintained on a 12-hour light/dark cycle and given access to food and water ad libitum. All procedures involving animals were performed in accordance with standards established in the Guide for the Care and Use of Laboratory Animals as adopted by the U.S. National Institutes of Health. All animal protocols were approved by the Biogen Institutional Animal Care and Use Committee, which is accredited by the Association for Assessment and Accreditation of Laboratory Animal Care International.

### Study Design:

Compound 47-Ent1 (10 or 50 mg/kg) was dosed in a single, oral dose in a vehicle of 2% hydroxypropyl methyl cellulose/1% Tween to C57BL/6 (wt) mice. Brain and kidney were collected at 2 or 6 hours after dosing for RNA analysis of Nrf2 target genes.

### Tissue RNA Extraction

For RNA preparation, frozen tissues were placed in 2 mL RNAse-free 96-well blocks with 1.5 ml QIAzol Lysis Reagent (QIAgen) and a 3.2 mm stainless steel bead. Tissues were disrupted for four cycles of 45 seconds in a Mini-Beadbeater (BioSpec Products, Bartlesville, Okla.). RNA was extracted in chloroform and the aqueous phase was mixed with an equal volume of 70% ethanol. Extracted RNA was applied to RNeasy 96 plates and purified by the spin method according to the manufacturer's protocol (RNeasy 96 Universal Tissue Protocol, QIAgen, Hilden Germany).

Quantitative Real-Time PCR (qRT-PCR)

qRT-PCR was performed from total mRNA isolated from tissues and reverse-transcribed into cDNA according to manufacturer protocols (Life Technologies, Carlsbad, Calif.). 20× Taqman target gene mouse primer/probe sets (see table below) were mixed with cDNA and 2× Taqman Universal Master Mix to a final volume of 20 uL. All final reactions contained 100 ng of cDNA, 900 nM of each primer, and 250 nM TaqMan® probes and were cycled on a QuantStudio™ 12K Flex system (Life Technologies). All samples were measured in triplicate using beta actin as a normalizing gene. Final analysis was performed using the comparative CT method to calculate fold changes and samples were normalized relative to wild type vehicle control at each time point.

Taqman Primer/Probe Assays (Life Technologies)

| Gene | Taqman Assay ID |
|---|---|
| HMOX1 | Mm00516005_m1 |
| OSGIN1 | Mm00660947_m1 |
| NQO1 | Mm01253561_m1 |
| AKR1B8 | Mm00484314_m1 |
| ACTB | Mm02619580_g1 |
| CBR3 | Mm00557339_m1 |

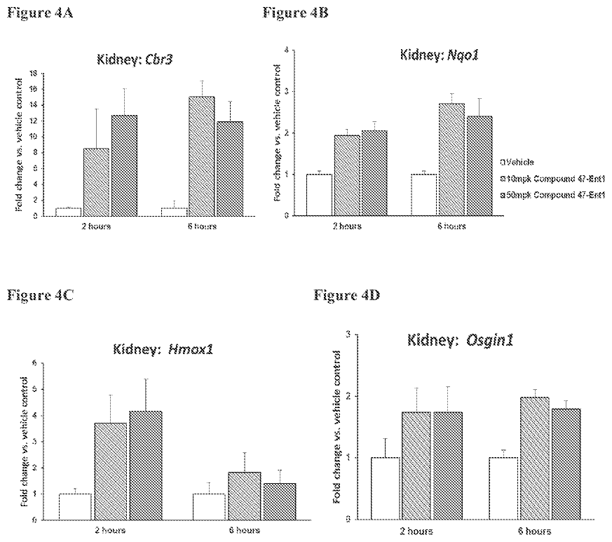

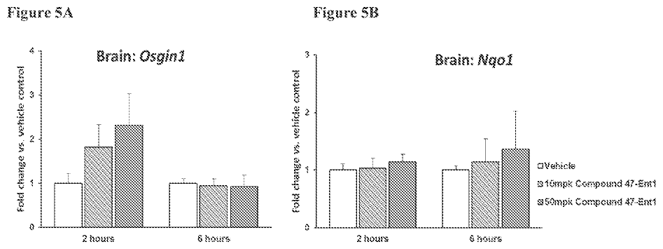

As shown in FIGS. 4A to 4D, Compound 47-Ent1 at doses of 10 and 50 mg/kg increases the expression of Cbr3, Nqo1, and Osgin1 in kidney at 2 hours and 6 hours after dosing, and Hmox1 at 2 hours. As shown in FIGS. 5A and 5B, Compound 47-Ent1 at does of 10 and 50 mg/kg increases the expression of Osgin1 in brain at 2 hours after dosing

Other embodiments are within the scope of the following claims.

What is claimed is:

1. A compound represented by Formula A: 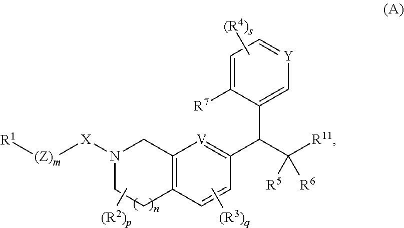
or a pharmaceutically acceptable salt thereof, wherein
V is CH or N;
R¹ is a 3 to 12-membered carbocyclyl, a 3 to 12-membered heterocyclyl, or —N(R^{1a})₂, wherein the 3 to 12-membered carbocyclyl and 3 to 12-membered heterocyclyl are each optionally substituted with one or more R¹⁵;
X is —C(O)— or —S(O)₂—;
R² is halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{2a}, —C(S)R^{2a}, —C(O)OR^{2a}, —C(S)SR^{2a}, —C(O)SR^{2a}, —C(S)OR^{2a}, —SC(O)R^{2a}, —OC(S)R^{2a}, —SC(S)R^{2a}, —C(O)N(R^{2a})₂, —OR^{2a}, —SR^{2a}, —N(R^{2a})₂, —N(R^{2a})OR^{2a}, —N(R^{2a})S(O)₂R^{2a}, —N(R^{2a})C(O)R^{2a}, —N(R^{2a})N(R^{2a})₂, —N(R^{2a})C(O)OR^{2a}, —N(R^{2a})C(O)N(R^{2a})₂, —S(O)₂R^{2a}, —S(O)R^{2a}, —S(O)N(R^{2a})₂, —S(O)₂N(R^{2a})₂, —N⁺(R^{2a})₃, —S⁺(R^{2a})₂, or —Si(R^{2a})₃ or two R² groups, attached to non-adjacent ring carbon atoms and taken together with the two non-adjacent ring carbon atoms, form a non-saturated heterocyclic, bridged bicyclyl, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R²⁵, and wherein the non-saturated heterocyclic, bridged bicyclyl is optionally substituted with one or more R⁹;
R³ is halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{3a}, —C(S)R^{3a}, —C(O)OR^{3a}, —C(S)SR^{3a}, —C(O)SR^{3a}, —C(S)OR^{3a}, —SC(O)R^{3a}, —OC(S)R^{3a}, —SC(S)R^{3a}, —C(O)N(R^{3a})₂, —OR^{3a}, —SR^{3a}, —N(R^{3a})₂, —N(R^{3a})OR^{3a}, —N(R^{3a})S(O)₂R^{3a}, —N(R^{3a})C(O)R^{3a}, —N(R^{3a})N(R^{3a})₂, —N(R^{3a})C(O)OR^{3a}, —N(R^{3a})C(O)N(R^{3a})₂, —S(O)₂R^{3a}, —S(O)R^{3a}, —S(O)N(R^{3a})₂, —S(O)₂N(R^{3a})₂, —N⁺(R^{3a})₃, —S⁺(R^{3a})₂, or —Si(R^{3a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R³⁵;
R⁴ is halo, —NO₂, —CN, —N₃, C₁₋₁₂alkyl, C₂₋₁₂alkenyl, C₂₋₁₂alkynyl, —C(O)R^{4a}, —C(S)R^{4a}, —C(O)OR^{4a}, —C(S)SR^{4a}, —C(O)SR^{4a}, —C(S)OR^{4a}, —SC(O)R^{4a}, —OC(S)R^{4a}, —SC(S)R^{4a}, —C(O)N(R^{4a})₂, —OR^{4a}, —SR^{4a}, —N(R^{4a})₂, —N(R^{4a})OR^{4a}, —N(R^{4a})S(O)₂R^{4a}, —N(R^{4a})C(O)R^{4a}, —N(R^{4a})N(R^{4a})₂, —N(R^{4a})C(O)OR^{4a}, —N(R^{4a})C(O)N(R^{4a})₂, —S(O)₂R^{4a}, —S(O)R^{4a}, —S(O)N(R^{4a})₂, —S(O)₂N(R^{4a})₂, —N⁺(R^{4a})₃, —S⁺(R^{4a})₂, or —Si(R^{4a})₃; or two R⁴ groups, attached to adjacent ring carbon atoms and taken together with the two adjacent ring carbon atoms, form triazolyl, 2,5-dihydrofuranyl, 2,3-dihydro-1,4-dioxinyl, 3,4-dihydro-2,4-pyranyl, 1,2,3,6-tetrahydropyridinyl, 1H-imidazolyl or pyrazinyl, wherein the C₁₋₁₂alkyl, C₂₋₁₂alkenyl, and C₂₋₁₂alkynyl are each optionally substituted with one or more R⁴⁵, and wherein the triazolyl, 2,5-dihydrofuranyl, 2,3-dihydro-1,4-dioxinyl, 3,4-dihydro-2,4-pyranyl, 1,2,3,6-tetrahydropyridinyl, 1H-imidazolyl and pyrazinyl are each optionally substituted with one or more R⁹;
R⁵ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{5a}, —C(S)R^{5a}, —C(O)OR^{5a}, —C(S)SR^{5a}, —C(O)SR^{5a}, —C(S)OR^{5a}, —SC(O)R^{5a}, —OC(S)R^{5a}, —SC(S)R^{5a}, —C(O)N(R^{5a})₂, —OR^{5a}, —SR^{5a}, —N(R^{5a})₂, —N(R^{5a})OR^{5a}, —N(R^{5a})S(O)₂R^{5a}, —N(R^{5a})C(O)R^{5a}, —N(R^{5a})N(R^{5a})₂, —N(R^{5a})C(O)OR^{5a}, —N(R^{5a})C(O)N(R^{5a})₂, —S(O)₂R^{5a}, —S(O)R^{5a}, —S(O)N(R^{5a})₂, —S(O)₂N(R^{5a})₂, —N⁺(R^{5a})₃, —S⁺(R^{5a})₂, or —Si(R^{5a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁵⁵;
R⁶, in each occurrence, is independently H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{6a}, —C(S)R^{6a}, —C(O)OR^{6a}, —C(S)SR^{6a}, —C(O)SR^{6a}, —C(S)OR^{6a}, —SC(O)R^{6a}, —OC(S)R^{6a}, —SC(S)R^{6a}, —C(O)N(R^{6a})₂, —OR^{6a}, —SR^{6a}, —N(R^{6a})₂, —N(R^{6a})OR^{6a}, —N(R^{6a})S(O)₂R^{6a}, —N(R^{6a})C(O)R^{6a}, —N(R^{6a})N(R^{6a})₂, —N(R^{6a})C(O)OR^{6a}, —N(R^{6a})C(O)N(R^{6a})₂, —S(O)₂R^{6a}, —S(O)R^{6a}, —S(O)N(R^{6a})₂, —S(O)₂N(R^{6a})₂, —N⁺(R^{6a})₃, —S⁺(R^{6a})_{2,} or —Si(R^{6a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁶⁵;
R⁷ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{7a}, —C(S)R^{7a}, —C(O)OR^{7a}, —C(S)SR^{7a}, —C(O)SR^{7a}, —C(S)OR^{7a}, —SC(O)R^{7a}, —OC(S)R^{7a}, —SC(S)R^{7a}, —C(O)N(R^{7a})₂, —OR^{7a}, —SR^{7a}, —N(R^{7a})₂, —N(R^{7a})OR^{7a}, —N(R^{7a})S(O)₂R^{7a}, —N(R^{7a})C(O)R^{7a}, —N(R^{7a})N(R^{7a})₂, —N(R^{7a})C(O)OR^{7a}, —N(R^{7a})C(O)N(R^{7a})₂, —S(O)₂R^{7a}, —S(O)R^{7a}, —S(O)N(R^{7a})₂, —S(O)₂N(R^{7a})₂, —N⁺(R^{7a})₃, —S⁺(R^{7a})_{2,} or —Si(R^{7a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁷⁵;
Y is N or CR⁸, wherein R⁸ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{8a}, —C(S)R^{8a}, —C(O)OR^{8a}, —C(S)SR^{8a}, —C(O)SR^{8a}, —C(S)OR^{8a}, —SC(O)R^{8a}, —OC(S)R^{8a}, —SC(S)R^{8a}, —C(O)N(R^{8a})₂, —OR^{8a}, —SR^{8a}, —N(R^{8a})₂, —N(R^{8a})OR^{8a}, —N(R^{8a})S(O)₂R^{8a}, —N(R^{8a})C(O)R^{8a}, —N(R^{8a})N(R^{8a})₂, —N(R^{8a})C(O)OR^{8a}, —N(R^{8a})C(O)N(R^{8a})₂, —S(O)₂R^{8a}, —S(O)R^{8a}, —S(O)N(R^{8a})₂, —S(O)₂N(R^{8a})₂, —N⁺(R^{8a})₃, —S⁺(R^{8a})_{2,} or —Si(R^{8a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁸⁵;
R⁹ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{9a}, —C(S)R^{9a}, —C(O)OR^{9a}, —C(S)SR^{9a}, —C(O)SR^{9a}, —C(S)OR^{9a}, —SC(O)R^{9a}, —OC(S)R^{9a}, —SC(S)R^{9a}, —C(O)N(R^{9a})₂, —OR^{9a}, —SR^{9a}, —N(R^{9a})₂, —N(R^{9a})OR^{9a}, —N(R^{9a})S(O)₂R^{9a}, —N(R^{9a})C(O)R^{9a}, —N(R^{9a})N(R^{9a})₂, —N(R^{9a})C(O)OR^{9a}, —N(R^{9a})C(O)N(R^{9a})₂, —S(O)₂R^{9a}, —S(O)R^{9a}, —S(O)N(R^{9a})₂, —S(O)₂N(R^{9a})₂, —N⁺(R^{9a})₃, —S⁺(R^{9a})_{2,} or —Si(R^{9a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁹⁵;
R¹¹ is —C(O)R^{11a}, —CN, —NHCOH and —NHS(O)₂CH₃, wherein R^{11a} is selected from the group consisting of —OR¹¹⁵, —N(OH)R¹¹⁵, —CH₂OH, —NHNH₂, —N(R¹¹⁵)OR¹¹⁵, —NHR¹¹⁵ and —ONHR¹¹⁵; and wherein R¹¹⁵, in each occurrence, is independently H or C₁₋₄alkyl;
Z is C(R¹⁰)₂, wherein R¹⁰ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{10a}, —C(S)R^{10a}, —C(O)OR^{10a}, —C(S)SR^{10a}, —C(O)SR^{10a}, —C(S)OR^{10a}, —SC(O)R^{10a}, —OC(S)R^{10a}, —SC(S)R^{10a}, —C(O)N(R^{10a})₂, —OR^{10a}, —SR^{10a}, —N(R^{10a})₂, —N(R^{10a})OR^{10a}, —N(R^{10a})S(O)₂R^{10a}, —N(R^{10a})C(O)R^{10a}, —N(R^{10a})N(R^{10a})₂, —N(R^{10a})C(O)OR^{10a}, —N(R^{10a})C(O)N(R^{10a})₂, —S(O)₂R^{10a}, —S(O)R^{10a}, —S(O)N(R^{10a})₂, —S(O)₂N(R^{10a})₂, —N⁺(R^{10a})₃, —S⁺(R^{10a})₂, or —Si(R^{10a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R¹⁰⁵
R^{1a}, R^{2a}, R^{3a}, R^{4a}, R^{5a}, R^{6a}, R^{7a}, R^{8a}, R^{9a}, and R^{10a}, in each occurrence, are independently H, C₁₋₁₂alkyl, C₂₋₁₂alkenyl, C₂₋₁₂alkynyl, C₁₋₁₂alkoxy, C₁₋₁₂acyl, —Si(C₁₋₁₂alkyl)₃, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, C₁₋₁₂acyl, 3 to 12-membered carbocyclyl, and 3 to 12-membered heterocyclyl are each optionally substituted with one or more R¹⁷;
R¹⁵, R²⁵, R³⁵, R⁴⁵, R⁵⁵, R⁶⁵, R⁷⁵, R⁸⁵, R⁹⁵, and R¹⁰⁵, in each occurrence, are independently halo, —OH, —CN, C₁₋₁₂alkyl, C₂₋₁₂alkenyl, C₂₋₁₂alkynyl, C₁₋₁₂alkoxy, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, 3 to 12-membered carbocyclyl, and 3 to 12-membered heterocyclyl are each optionally substituted with one or more R¹⁹; and
R¹⁷ and R¹⁹, in each occurrence, are independently halo, —OH, —CN, C₁₋₁₂alkyl, C₂₋₁₂alkenyl, C₂₋₁₂alkynyl, C₁₋₁₂alkoxy, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, 3 to 12-membered carbocyclyl and 3 to 12-membered heterocyclyl are each optionally substituted with one or more groups independently selected from halo, —OH, and C₁₋₄alkoxy;
m is 0 or 1,
n is 1 or 2;
p is 0 or an integer from 1 to 8;
q is 0 or an integer from 1 to 3; and
s is an integer from 1 to 3.

2. A compound represented by Formula I: 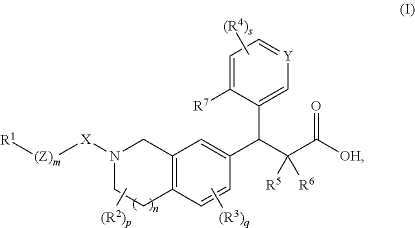
or a pharmaceutically acceptable salt thereof, wherein
R¹ is a 3 to 12-membered carbocyclyl, a 3 to 12-membered heterocyclyl, or —N(R^{1a})₂, wherein the 3 to 12-membered carbocyclyl and 3 to 12-membered heterocyclyl are each optionally substituted with one or more R¹⁵;
X is —C(O)— or —S(O)₂—;
R² is halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{2a}, —C(S)R^{2a}, —C(O)OR^{2a}, —C(S)SR^{2a}, —C(O)SR^{2a}, —C(S)OR^{2a}, —SC(O)R^{2a}, —OC(S)R^{2a}, —SC(S)R^{2a}, —C(O)N(R^{2a})₂, —OR^{2a}, —SR^{2a}, —N(R^{2a})₂, —N(R^{2a})OR^{2a}, —N(R^{2a})S(O)₂R^{2a}, —N(R^{2a})C(O)R^{2a}, —N(R^{2a})N(R^{2a})₂, —N(R^{2a})C(O)OR^{2a}, —N(R^{2a})C(O)N(R²)₂, —S(O)₂R^{2a}, —S(O)R^{2a}, —S(O)N(R^{2a})₂, —S(O)₂N(R^{2a})₂, —N⁺(R^{2a})₃, —S⁺(R^{2a})₂, or —Si(R^{2a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R²⁵;
R³ is halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{3a}, —C(S)R^{3a}, —C(O)OR^{3a}, —C(S)SR^{3a}, —C(O)SR^{3a}, —C(S)OR^{3a}, —SC(O)R^{3a}, —OC(S)R^{3a}, —SC(S)R^{3a}, —C(O)N(R^{3a})₂, —OR^{3a}, —SR^{3a}, —N(R^{3a})₂, —N(R^{3a})OR^{3a}, —N(R^{3a})S(O)₂R^{3a}, —N(R^{3a})C(O)R^{3a}, —N(R^{3a})N(R^{3a})₂, —N(R^{3a})C(O)OR^{3a}, —N(R^{3a})C(O)N(R^{3a})₂, —S(O)₂R^{3a}, —S(O)R^{3a}, —S(O)N(R^{3a})₂, —S(O)₂N(R^{3a})₂, —N⁺(R^{3a})₃, —S⁺(R^{3a})₂, or —Si(R^{3a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R³⁵;
R⁴ is halo, —NO₂, —CN, —N₃, C₁₋₁₂alkyl, C₂₋₁₂alkenyl, C₂₋₁₂alkynyl, —C(O)R^{4a}, —C(S)R^{4a}, —C(O)OR^{4a}, —C(S)SR^{4a}, —C(O)SR^{4a}, —C(S)OR^{4a}, —SC(O)R^{4a}, —OC(S)R^{4a}, —SC(S)R^{4a}, —C(O)N(R^{4a})₂, —OR^{4a}, —SR^{4a}, —N(R^{4a})₂, —N(R^{4a})OR^{4a}, —N(R^{4a})S(O)₂R^{4a}, —N(R^{4a})C(O)R^{4a}, —N(R^{4a})N(R^{4a})₂, —N(R^{4a})C(O)OR^{4a}, —N(R^{4a})C(O)N(R^{4a})₂, —S(O)₂R^{4a}, —S(O)R^{4a}, —S(O)N(R^{4a})₂, —S(O)₂N(R^{4a})₂, —N⁺(R^{4a})₃, —S⁺(R^{4a})₂, or —Si(R^{4a})₃; or two R⁴ groups, attached to adjacent ring carbon atoms and taken together with the two adjacent ring carbon atoms, form triazolyl, wherein the C₁₋₁₂alkyl, C₂₋₁₂alkenyl, and C₂₋₁₂alkynyl are each optionally substituted with one or more R⁴⁵, and wherein the triazolyl is optionally substituted with one or more R⁹;
R⁵ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{5a}, —C(S)R^{5a}, —C(O)OR^{5a}, —C(S)SR^{5a}, —C(O)SR^{5a}, —C(S)OR^{5a}, —SC(O)R^{5a}, —OC(S)R^{5a}, —SC(S)R^{5a}, —C(O)N(R^{5a})₂, —OR^{5a}, —SR^{5a}, —N(R^{5a})₂, —N(R^{5a})OR^{5a}, —N(R^{5a})S(O)₂R^{5a}, —N(R^{5a})C(O)R^{5a}, —N(R^{5a})N(R^{5a})₂, —N(R^{5a})C(O)OR^{5a}, —N(R^{5a})C(O)N(R^{5a})₂, —S(O)₂R^{5a}, —S(O)R^{5a}, —S(O)N(R^{5a})₂, —S(O)₂N(R^{5a})₂, —N⁺(R^{5a})₃, —S⁺(R^{5a})₂, or —Si(R^{5a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁵⁵;
R⁶, in each occurrence, is independently H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{6a}, —C(S)R^{6a}, —C(O)OR^{6a}, —C(S)SR^{6a}, —C(O)SR^{6a}, —C(S)OR^{6a}, —SC(O)R^{6a}, —OC(S)R^{6a}, —SC(S)R^{6a}, —C(O)N(R^{6a})₂, —OR^{6a}, —SR^{6a}, —N(R^{6a})₂, —N(R^{6a})OR^{6a}, —N(R^{6a})S(O)₂R^{6a}, —N(R^{6a})C(O)R^{6a}, —N(R^{6a})N(R^{6a})₂, —N(R^{6a})C(O)OR^{6a}, —N(R^{6a})C(O)N(R^{6a})₂, —S(O)₂R^{6a}, —S(O)R^{6a}, —S(O)N(R^{6a})₂, —S(O)₂N(R^{6a})₂, —N⁺(R^{6a})₃, —S⁺(R^{6a})_{2,} or —Si(R^{6a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁶⁵;
R⁷ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{7a}, —C(S)R^{7a}, —C(O)OR^{7a}, —C(S)SR^{7a}, —C(O)SR^{7a}, —C(S)OR^{7a}, —SC(O)R^{7a}, —OC(S)R^{7a}, —SC(S)R^{7a}, —C(O)N(R^{7a})₂, —OR^{7a}, —SR^{7a}, —N(R^{7a})₂, —N(R^{7a})OR^{7a}, —N(R^{7a})S(O)₂R^{7a}, —N(R^{7a})C(O)R^{7a}, —N(R^{7a})N(R^{7a})₂, —N(R^{7a})C(O)OR^{7a}, —N(R^{7a})C(O)N(R^{7a})₂, —S(O)₂R^{7a}, —S(O)R^{7a}, —S(O)N(R^{7a})₂, —S(O)₂N(R^{7a})₂, —N⁺(R^{7a})₃, —S⁺(R^{7a})_{2,} or —Si(R^{7a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁷⁵;
Y is N or CR⁸, wherein R⁸ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{8a}, —C(S)R^{8a}, —C(O)OR^{8a}, —C(S)SR^{8a}, —C(O)SR^{8a}, —C(S)OR^{8a}, —SC(O)R^{8a}, —OC(S)R^{8a}, —SC(S)R^{8a}, —C(O)N(R^{8a})₂, —OR^{8a}, —SR^{8a}, —N(R^{8a})₂, —N(R^{8a})OR^{8a}, —N(R^{8a})S(O)₂R^{8a}, —N(R^{8a})C(O)R^{8a}, —N(R^{8a})N(R^{8a})₂, —N(R^{8a})C(O)OR^{8a}, —N(R^{8a})C(O)N(R^{8a})₂, —S(O)₂R^{8a}, —S(O)R^{8a}, —S(O)N(R^{8a})₂, —S(O)₂N(R^{8a})₂, —N⁺(R^{8a})₃, —S⁺(R^{8a})_{2,} or —Si(R^{8a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁸⁵;
R⁹ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{9a}, —C(S)R^{9a}, —C(O)OR^{9a}, —C(S)SR^{9a}, —C(O)SR^{9a}, —C(S)OR^{9a}, —SC(O)R^{9a}, —OC(S)R^{9a}, —SC(S)R^{9a}, —C(O)N(R^{9a})₂, —OR^{9a}, —SR^{9a}, —N(R^{9a})₂, —N(R^{9a})OR^{9a}, —N(R^{9a})S(O)₂R^{9a}, —N(R^{9a})C(O)R^{9a}, —N(R^{9a})N(R^{9a})₂, —N(R^{9a})C(O)OR^{9a}, —N(R^{9a})C(O)N(R^{9a})₂, —S(O)₂R^{9a}, —S(O)R^{9a}, —S(O)N(R^{9a})₂, —S(O)₂N(R^{9a})₂, —N⁺(R^{9a})₃, —S⁺(R^{9a})_{2,} or —Si(R^{9a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R⁹⁵;
Z is C(R¹⁰)₂, wherein R¹⁰ is H, halo, —NO₂, —CN, —N₃, C₁₋₁₂alkyl, C₂₋₁₂alkenyl, C₂₋₁₂alkynyl, a 3 to 12-membered carbocyclyl, a 3 to 12-membered heterocyclyl, —C(O)R^{10a}, —C(S)R^{10a}, —C(O)OR^{10a}, —C(S)SR^{10a}, —C(O)SR^{10a}, —C(S)OR^{10a}, —SC(O)R^{10a}, —OC(S)R^{10a}, —SC(S)R^{10a}, —C(O)N(R^{10a})₂, —OR^{10a}, —SR^{10a}, —N(R^{10a})₂, —N(R^{10a})OR^{10a}, —N(R^{10a})S(O)₂R^{10a}, —N(R^{10a})C(O)R^{10a}, —N(R^{10a})N(R^{10a})₂, —N(R^{10a})C(O)OR^{10a}, —N(R^{10a})C(O)N(R^{10a})₂, —S(O)₂R^{10a}, —S(O)R^{10a}, —S(O)N(R^{10a})₂, —S(O)₂N(R^{10a})₂, —N⁺(R^{10a})₃, —S⁺(R^{10a})₂, or —Si(R^{10a})₃, wherein the C₁₋₁₂alkyl, C₂₋₁₂alkenyl, C₂₋₁₂alkynyl, 3 to 12-membered carbocyclyl, and 3 to 12-membered heterocyclyl are each optionally substituted with one or more R¹⁰⁵
R^{1a}, R^{2a}, R^{3a}, R^{4a}, R^{5a}, R^{6a}, R^{7a}, R^{8a}, R^{9a}, and R^{10a}, in each occurrence, are independently H, C₁₋₁₂alkyl, C₂₋₁₂alkenyl, C₂₋₁₂alkynyl, C₁₋₁₂alkoxy, C₁₋₁₂acyl, —Si(C₁₋₁₂alkyl)₃, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, C₁₋₁₂acyl, 3 to 12-membered carbocyclyl, and 3 to 12-membered heterocyclyl are each optionally substituted with one or more R¹⁷;
R¹⁵, R²⁵, R³⁵, R⁴⁵, R⁵⁵, R⁶⁵, R⁷⁵, R⁸⁵, R⁹⁵, and R¹⁰⁵, in each occurrence, are independently halo, —OH, —CN, C₁₋₁₂alkyl, C₂₋₁₂alkenyl, C₂₋₁₂alkynyl, C₁₋₁₂alkoxy, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, 3 to 12-membered carbocyclyl, and 3 to 12-membered heterocyclyl are each optionally substituted with one or more R¹⁹; and
R¹⁷ and R¹⁹, in each occurrence, are independently halo, —OH, —CN, C₁₋₁₂alkyl, C₂₋₁₂alkenyl, C₂₋₁₂alkynyl, C₁₋₁₂alkoxy, a 3 to 12-membered carbocyclyl, or a 3 to 12-membered heterocyclyl, wherein the C₁₋₁₂alkyl, C₂₋₁₂alkenyl, C₂₋₁₂alkynyl, C₁₋₁₂alkoxy, 3 to 12-membered carbocyclyl and 3 to 12-membered heterocyclyl are each optionally substituted with one or more groups independently selected from halo, —OH, and C₁₋₄alkoxy;
m is 0 or 1,
n is 1 or 2;
p is 0 or an integer from 1 to 8;
q is 0 or an integer from 1 to 3; and
s is an integer from 1 to 3.

3. The compound of claim 1 or 2, or a pharmaceutically acceptable salt thereof, wherein p and q are each independently 0 or 1.

4. The compound of any one of claims 1-3, wherein the compound is represented by Formula II: 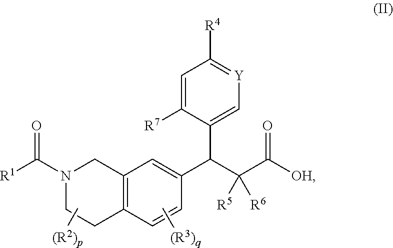
or a pharmaceutically acceptable salt thereof.

5. The compound of claim 4, wherein the compound is represented by Formula IIA or IIB: 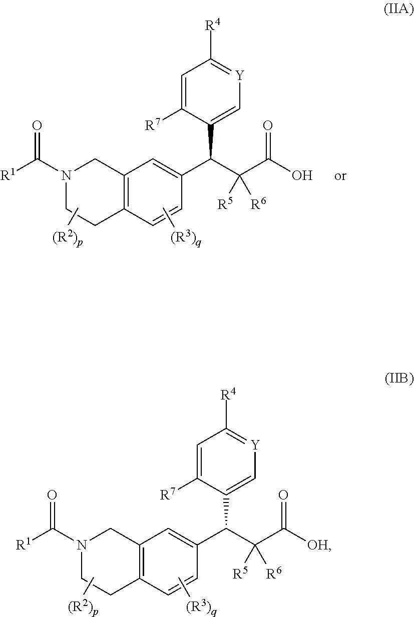
or a pharmaceutically acceptable salt thereof.

6. The compound of any one of claims 1-5, or a pharmaceutically acceptable salt thereof, wherein
R⁴ is —CN, —C(O)N(R^{4a})₂, or —OR^{4a}; and
R^{4a}, in each occurrence, is independently H or C₁₋₆alkyl, wherein the C₁₋₆alkyl is optionally substituted with one to six groups independently selected from halo, —CN, —OH, C₁₋₄alkyl, and C₁₋₄alkoxy.

7. The compound of claim 6, or a pharmaceutically acceptable salt thereof, wherein R⁴ is —CN, —C(O)N(R^{4a})₂, or —OR^{4a}, wherein R^{4a}, in each occurrence, is independently H or C₁₋₄alkyl.

8. The compound of any one of claims 1-3, wherein the compound is represented by Formula III: 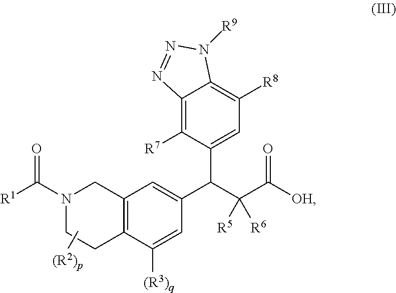
or a pharmaceutically acceptable salt thereof.

9. The compound of claim 8, wherein the compound is represented by Formula IIIA or IIIB: 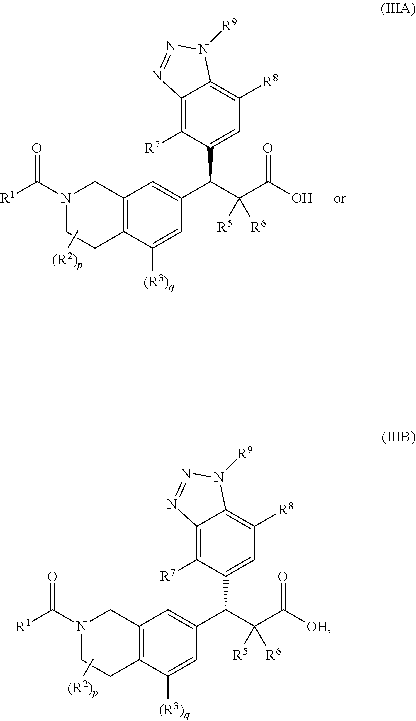
or a pharmaceutically acceptable salt thereof.

10. The compound of any one of claims 1-3, wherein the compound is represented by Formula IV: 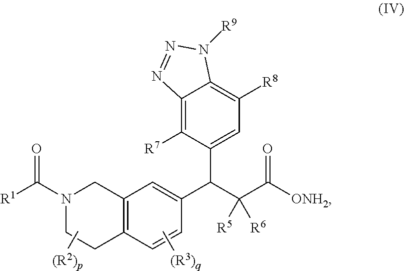
or a pharmaceutically acceptable salt thereof.

11. The compound of any one of claims 1-3 and 8-10, or a pharmaceutically acceptable salt thereof, wherein
R⁹ is H, halo, —CN, —OR^{9a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R⁹⁵;
R^{9a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷;
R⁹⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to six groups independently selected from halo, —OH, and C₁₋₄alkoxy; and
R¹⁷, in each occurrence, as an optional substituent of R^{9a}, is independently selected from halo, —CN, —OH, C₁₋₆alkyl, and C₁₋₆alkoxy, wherein the C₁₋₆alkyl and C₁₋₆alkoxy are each optionally substituted with one to six halo.

12. The compound of claim 11, or a pharmaceutically acceptable salt thereof, wherein R⁹ is H, halo, —OH, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy.

13. The compound of claim 12, or a pharmaceutically acceptable salt thereof, wherein R⁹ is H or C₁₋₄alkyl.

14. The compound of any one of claims 1-13, or a pharmaceutically acceptable salt thereof, wherein
R¹ is a 6 to 11-membered carbocyclyl, a 5 to 10-membered heterocyclyl, or —N(R^{1a})₂, wherein the 6 to 11-membered carbocyclyl and 5 to 10-membered heterocyclyl are each optionally substituted with one to eight R¹⁵;
R^{1a}, in each occurrence, is independently selected from H, C₁₋₁₂alkyl, C₂₋₁₂alkenyl, C₂₋₁₂alkynyl, and a 6 to 10-membered aromatic carbocyclyl, wherein the C₁₋₁₂alkyl, C₂₋₁₂alkenyl, C₂₋₁₂alkynyl, and 6 to 10-membered carbocyclyl are each optionally substituted with one to six R¹⁷;
R¹⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to six groups independently selected from halo, —OH, and C₁₋₄alkoxy; and
R¹⁷, in each occurrence, as an optional substituent of R^{1a}, is independently selected from halo, —CN, —OH, C₁₋₆alkyl, and C₁₋₆alkoxy, wherein the C₁₋₆alkyl and C₁₋₆alkoxy are each optionally substituted with one to six halo.

15. The compound of claim 14, or a pharmaceutically acceptable salt thereof, wherein
R¹ is benzofuran-2-yl, oxazolyl, pyrazolo [1,5-a]pyridine-2-yl, cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, thiazolyl or —N(R^{1a})₂, wherein the benzofuran-2-yl, oxazolyl, pyrazolo [1,5-a]pyridine-2-yl, cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, and thiazolyl are each optionally substituted with one to six groups independently selected from halo, —CN, —OH, C₁₋₄alkyl, and C₁₋₄alkoxy, wherein C₁₋₄alkyl and C₁₋₄alkoxy are optionally substituted with one to six halo.

16. The compound of claim 14, or a pharmaceutically acceptable salt thereof, wherein
R¹ is cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, thiazolyl or —N(R^{1a})₂, wherein the cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, and thiazolyl are each optionally substituted with one to six groups independently selected from halo, —CN, —OH, C₁₋₄alkyl, and C₁₋₄alkoxy, wherein C₁₋₄alkyl and C₁₋₄alkoxy are optionally substituted with one to six halo; and
R^{1a}, in each occurrence, is independently C₁₋₄alkyl or phenyl.

17. The compound of any one of claims 1-16, or a pharmaceutically acceptable salt thereof, wherein
R² is halo, —CN, —OR^{2a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R²⁵;
R^{2a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷;
R²⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to six groups independently selected from halo, —OH, and C₁₋₄alkoxy; and
R¹⁷, in each occurrence, as an optional substituent of R^{2a}, is independently selected from halo, —CN, —OH, C₁₋₆alkyl, and C₁₋₆alkoxy, wherein the C₁₋₆alkyl and C₁₋₆alkoxy are each optionally substituted with one to six halo.

18. The compound of claim 17, or a pharmaceutically acceptable salt thereof, wherein R² is halo, —OH, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy.

19. The compound of claim 18, or a pharmaceutically acceptable salt thereof, wherein R² is C₁₋₄alkyl.

20. The compound of any one of claims 1-19, or a pharmaceutically acceptable salt thereof, wherein
R³ is halo, —NO₂, —CN, —OR^{3a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R³⁵;
R^{3a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷;
R³⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to six groups independently selected from halo, —OH, and C₁₋₄alkoxy; and
R¹⁷, in each occurrence, as an optional substituent of R^{3a}, is independently selected from halo, —CN, —OH, C₁₋₆alkyl, and C₁₋₆alkoxy, wherein the C₁₋₆alkyl and C₁₋₆alkoxy are each optionally substituted with one to six halo.

21. The compound of claim 20, or a pharmaceutically acceptable salt thereof, wherein R³ is halo, —OH, —NO₂, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy.

22. The compound of claim 21, or a pharmaceutically acceptable salt thereof, wherein R³ is C₁₋₄alkyl or —NO₂.

23. The compound of any one of claims 1-22, or a pharmaceutically acceptable salt thereof, wherein
R⁵ is H, halo, —CN, —OR^{5a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R⁵⁵;
R^{5a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷;
R⁵⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to eight groups independently selected from halo, —OH, and C₁₋₄alkoxy; and
R¹⁷, in each occurrence, as an optional substituent of R^{5a}, is independently selected from halo, —CN, —OH, C₁₋₆alkyl, and C₁₋₆alkoxy, wherein the C₁₋₆alkyl and C₁₋₆alkoxy are each optionally substituted with one to six halo.

24. The compound of claim 23, or a pharmaceutically acceptable salt thereof, wherein R⁵ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy.

25. The compound of claim 24, or a pharmaceutically acceptable salt thereof, wherein R⁵ is H or C₁₋₄alkyl.

26. The compound of any one of claims 1-25 or a pharmaceutically acceptable salt thereof, wherein
R⁶ is H, halo, —CN, —OR^{6a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R⁶⁵;
R^{6a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷;
R⁶⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to eight groups independently selected from halo, —OH, and C₁₋₄alkoxy; and
R¹⁷, in each occurrence, as an optional substituent of R^{6a}, is independently selected from halo, —CN, —OH, C₁₋₆alkyl, and C₁₋₆alkoxy, wherein the C₁₋₆alkyl and C₁₋₆alkoxy are each optionally substituted with one to six halo.

27. The compound of claim 26, or a pharmaceutically acceptable salt thereof, wherein R⁶ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy.

28. The compound of claim 27, or a pharmaceutically acceptable salt thereof, wherein R⁶ is H or C₁₋₄alkyl.

29. The compound of any one of claims 1-28, or a pharmaceutically acceptable salt thereof, wherein
R⁷ is H, halo, —CN, —OR^{7a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R⁷⁵;
R^{7a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷;
R⁷⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to eight groups independently selected from halo, —OH, and C₁₋₄alkoxy; and
R¹⁷, in each occurrence, as an optional substituent of R^{7a}, is independently selected from halo, —CN, —OH, C₁₋₆alkyl, and C₁₋₆alkoxy, wherein the C₁₋₆alkyl and C₁₋₆alkoxy are each optionally substituted with one to six halo.

30. The compound of claim 29, or a pharmaceutically acceptable salt thereof, wherein R⁷ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy.

31. The compound of claim 30, or a pharmaceutically acceptable salt thereof, wherein R⁷ is H or C₁₋₄alkyl.

32. The compound of any one of claims 1-31, or a pharmaceutically acceptable salt thereof, wherein
R⁸ is H, halo, —CN, —OR^{8a}, C₁₋₁₂alkyl, C₂₋₁₂alkenyl, or C₂₋₁₂alkynyl, wherein the C₁₋₁₂alkyl, C₂₋₁₂alkenyl, and C₂₋₁₂alkynyl are each optionally substituted with one to eight R⁸⁵;
R^{8a} is selected from H, C₁₋₁₂alkyl, C₂₋₁₂alkenyl, and C₂₋₁₂alkynyl, wherein the C₁₋₁₂alkyl, C₂₋₁₂alkenyl, and C₂₋₁₂alkynyl are each optionally substituted with one to six R¹⁷;
R⁸⁵, in each occurrence, is independently selected from halo, —OH, —CN, C₁₋₁₂alkyl, C₂₋₁₂alkenyl, C₂₋₁₂alkynyl, and C₁₋₁₂alkoxy, wherein the C₁₋₁₂alkyl, C₂₋₁₂alkenyl, C₂₋₁₂alkynyl, and C₁₋₁₂alkoxy are each optionally substituted with one to six groups independently selected from halo, —OH, and C₁₋₄alkoxy; and
R¹⁷, in each occurrence, as an optional substituent of R^{8a}, is independently selected from halo, —CN, —OH, C₁₋₆alkyl, and C₁₋₆alkoxy, wherein the C₁₋₆alkyl and C₁₋₆alkoxy are each optionally substituted with one to six halo.

33. The compound of claim 32, or a pharmaceutically acceptable salt thereof, wherein R⁸ is H, halo, —OH, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy.

34. The compound of claim 33, or a pharmaceutically acceptable salt thereof, wherein R⁸ is H or C₁₋₄alkyl.

35. The compound of claim 1, 2, 8, 9 or 10, or a pharmaceutically acceptable salt thereof, wherein
R¹ is a 6 to 11-membered carbocyclyl, a 5 to 10-membered heterocyclyl, or —N(R^{1a})₂, wherein the 6 to 11-membered carbocyclyl and 5 to 10-membered heterocyclyl are each optionally substituted with one to six groups selected from halo, —CN, —OH, C₁₋₆alkyl, and C₁₋₆alkoxy, wherein the C₁₋₆alkyl and C₁₋₆alkoxy, in each occurrence, are optionally substituted with one to six halo; and wherein R^{1a}, in each occurrence, is independently selected from C₁₋₆alkyl and a 6 to 10-membered aromatic carbocyclyl, wherein the C₁₋₆alkyl and 6 to 10-membered carbocyclyl are each optionally substituted with one to six groups selected from halo, —CN, —OH, C₁₋₄alkyl, and C₁₋₄alkoxy;
X is —C(O)—;
R² is halo, —OH, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy;
R³ is halo, —OH, —NO₂, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy;
two R⁴ groups, attached to adjacent ring carbon atoms and taken together with the two adjacent ring carbon atoms, form triazolyl, wherein the triazolyl is optionally substituted with R⁹;
R⁵ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy;
R⁶ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy;
R⁷ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy;
Y is CR⁸;
R⁸ is H, halo, —OH, —CN, C₁₋₆alkyl, or C₁₋₆alkoxy, wherein the C₁₋₆alkyl and C₁₋₆alkoxy are each optionally substituted by one to six groups independently selected from halo, —CN, —OH, C₁₋₄alkyl, and C₁₋₄alkoxy;
R⁹ is H, halo, —OH, —CN, C₁₋₆alkyl, or C₁₋₆alkoxy, wherein the C₁₋₆alkyl and C₁₋₆alkoxy are each optionally substituted with one to six groups independently selected from halo, —CN, —OH, C₁₋₄alkyl, and C₁₋₄alkoxy;
m is 0;
n is 1;
p is 0 or 1;
q is 0 or 1; and
s is 2.

36. The compound of claim 35, or a pharmaceutically acceptable salt thereof, wherein
R¹ is cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, thiazolyl or —N(R^{1a})₂, wherein the cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, and thiazolyl are each optionally substituted with one to six groups independently selected from halo, —CN, —OH, C₁₋₄alkyl, and C₁₋₄alkoxy, wherein C₁₋₄alkyl, and C₁₋₄alkoxy are optionally substituted with one to six halo; and wherein R^{1a}, in each occurrence, is independently C₁₋₄alkyl or phenyl;
X is —C(O)—;
R² is C₁₋₄alkyl, wherein the C₁₋₄alkyl is optionally substituted with one to three groups independently selected from halo, —OH, and C₁₋₄alkoxy;
R³ is —NO₂ or C₁₋₄alkyl, wherein the C₁₋₄alkyl is optionally substituted with one to three groups independently selected from halo, —OH, and C₁₋₄alkoxy;
two R⁴ groups, attached to adjacent ring carbon atoms and taken together with the two adjacent ring carbon atoms, form triazolyl, wherein the triazolyl is optionally substituted with R⁹;
R⁵ is H or C₁₋₄alkyl, wherein the C₁₋₄alkyl is optionally substituted by one to three groups independently selected from halo, —OH, and C₁₋₄alkoxy;
R⁶ is H or C₁₋₄alkyl, wherein the C₁₋₄alkyl is optionally substituted by one to three groups independently selected from halo, —OH, and C₁₋₄alkoxy;
R⁷ is H or C₁₋₄alkyl, wherein the C₁₋₄alkyl is optionally substituted by one to three groups independently selected from halo, —OH, and C₁₋₄alkoxy;
Y is CR⁸;
R⁸ is H or C₁₋₄alkyl, wherein the C₁₋₄alkyl is optionally substituted by one to three groups independently selected from halo, —OH, and C₁₋₄alkoxy;
R⁹ is H or C₁₋₄alkyl;
m is 0;
n is 1;
p is 0 or 1;
q is 0 or 1; and
s is 2.

37. The compound of claim 36, or a pharmaceutically acceptable salt thereof, wherein
R¹ is cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, thiazolyl, or —N(R^{1a})₂, wherein the cyclohexyl, naphthalyl, phenyl, bicyclo[2.2.1]heptyl, decahydro-2,7-methanonaphthyl, morpholinyl, piperidinyl, benzimidazolyl, imidazolyl, indolyl, pyridyl, pyrimidyl, pyrazinyl, pyrazolyl, isoxazolyl, quinolinyl, and thiazolyl are each optionally substituted with one to six groups selected from halo, —CN, —OH, methyl, isopropyl, t-butyl, and methoxy, wherein the methyl, isopropyl, t-butyl, and methoxy, in each occurrence, are optionally substituted with one to three halo, and wherein one R^{1a} is C₁₋₄alkyl and the other is phenyl;
X is —C(O)—;
R² is C₁₋₄alkyl;
R³ is C₁₋₄alkyl or —NO₂;
two R⁴ groups, attached to adjacent ring carbon atoms and taken together with the two adjacent ring carbon atoms, form triazolyl, wherein the triazolyl is optionally substituted with R⁹;
R⁵ is H or C₁₋₄alkyl;
R⁶ is H or C₁₋₄alkyl;
R⁷ is H or C₁₋₄alkyl;
Y is CR⁸;
R⁸ is H;
R⁹ is C₁₋₄alkyl;
m is 0;
n is 1;
p is 0 or 1;
q is 0 or 1; and
s is 2.

38. The compound of claim 37, or a pharmaceutically acceptable salt thereof, wherein
R¹ is phenyl, wherein the phenyl is optionally substituted with one to four groups selected from methyl and fluoro;
X is —C(O)—;
R² is methyl;
R³ is methyl;
two R⁴ groups, attached to adjacent ring carbon atoms and taken together with the two adjacent ring carbon atoms, form triazolyl, wherein the triazolyl is optionally substituted with methyl or ethyl;
R⁵ is H or methyl;
R⁶ is H or methyl;
R⁷ is H or methyl;
Y is CR⁸;
R⁸ is H;
m is 0;
n is 1;
p is 0 or 1;
q is 0 or 1; and
s is 2.

39. The compound of claim 1, selected from the group consisting of:
3-(1-ethyl-4-methyl-benzotriazol-5-yl)-3-[2-(4-methylbenzoyl)-3,4-dihydro-1H-isoquinolin-7-yl]propanoic acid;
3-(1-ethyl-4-methyl-benzotriazol-5-yl)-3-[2-(4-methoxybenzoyl)-3,4-dihydro-1H-isoquinolin-7-yl]propanoic acid;
3-(1-ethyl-4-methyl-benzotriazol-5-yl)-3-[2-(4-hydroxybenzoyl)-3,4-dihydro-1H-isoquinolin-7-yl]propanoic acid;
3-[2-(4-chlorobenzoyl)-3,4-dihydro-1H-isoquinolin-7-yl]-3-(1-ethyl-4-methyl-benzotriazol-5-yl)propanoic acid;
3-[2-(4-tert-butylbenzoyl)-3,4-dihydro-1H-isoquinolin-7-yl]-3-(1-ethyl-4-methyl-benzotriazol-5-yl)propanoic acid;
3-[2-(cyclohexanecarbonyl)-3,4-dihydro-1H-isoquinolin-7-yl]-3-(1-ethyl-4-methyl-benzotriazol-5-yl)propanoic acid;
3-[2-(2-chlorobenzoyl)-3,4-dihydro-1H-isoquinolin-7-yl]-3-(1-ethyl-4-methyl-benzotriazol-5-yl)propanoic acid;
3-[2-(2,4-dichlorobenzoyl)-3,4-dihydro-1H-isoquinolin-7-yl]-3-(1-ethyl-4-methyl-benzotriazol-5-yl)propanoic acid;
3-[2-(2,5-dichlorobenzoyl)-3,4-dihydro-1H-isoquinolin-7-yl]-3-(1-ethyl-4-methyl-benzotriazol-5-yl)propanoic acid;
3-[2-(3-chlorobenzoyl)-3,4-dihydro-1H-isoquinolin-7-yl]-3-(1-ethyl-4-methyl-benzotriazol-5-yl)propanoic acid;
3-[2-(3,4-dichlorobenzoyl)-3,4-dihydro-1H-isoquinolin-7-yl]-3-(1-ethyl-4-methyl-benzotriazol-5-yl)propanoic acid;
(3S)-3-[2-(3,4-dichlorobenzoyl)-3,4-dihydro-1H-isoquinolin-7-yl]-3-(1-ethyl-4-methyl-benzotriazol-5-yl)propanoic acid;
(3R)-3-[2-(3,4-dichlorobenzoyl)-3,4-dihydro-1H-isoquinolin-7-yl]-3-(1-ethyl-4-methyl-benzotriazol-5-yl)propanoic acid;
3-[2-(3,5-dichlorobenzoyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-(pyrimidine-2-carbonyl)-3,4-dihydro-1H-isoquinolin-7-yl]propanoic acid;
3-(1-ethyl-4-methyl-benzotriazol-5-yl)-3-[2-(pyrazine-2-carbonyl)-3,4-dihydro-1H-isoquinolin-7-yl]propanoic acid;
3-(1-ethyl-4-methyl-benzotriazol-5-yl)-3-[2-(pyrimidine-4-carbonyl)-3,4-dihydro-1H-isoquinolin-7-yl]propanoic acid;
3-(1-ethyl-4-methyl-benzotriazol-5-yl)-3-[2-(pyridine-2-carbonyl)-3,4-dihydro-1H-isoquinolin-7-yl]propanoic acid;
3-(1-ethyl-4-methyl-benzotriazol-5-yl)-3-[2-(pyridine-3-carbonyl)-3,4-dihydro-1H-isoquinolin-7-yl]propanoic acid;
3-(1-ethyl-4-methyl-benzotriazol-5-yl)-3-[2-(pyridine-4-carbonyl)-3,4-dihydro-1H-isoquinolin-7-yl]propanoic acid;
3-(1-ethyl-4-methyl-benzotriazol-5-yl)-3-[2-(1-methylpyrazole-4-carbonyl)-3,4-dihydro-1H-isoquinolin-7-yl]propanoic acid;
3-(1-ethyl-4-methyl-benzotriazol-5-yl)-3-[2-(1-methylpyrazole-3-carbonyl)-3,4-dihydro-1H-isoquinolin-7-yl]propanoic acid;
3-(1-ethyl-4-methyl-benzotriazol-5-yl)-3-[2-(1-methylimidazole-4-carbonyl)-3,4-dihydro-1H-isoquinolin-7-yl]propanoic acid;
3-(1-ethyl-4-methyl-benzotriazol-5-yl)-3-[2-(isoxazole-3-carbonyl)-3,4-dihydro-1H-isoquinolin-7-yl]propanoic acid;
3-(1-ethyl-4-methyl-benzotriazol-5-yl)-3-[2-(thiazole-4-carbonyl)-3,4-dihydro-1H-isoquinolin-7-yl]propanoic acid;
3-(1-ethyl-4-methyl-benzotriazol-5-yl)-3-[2-(3-methylbenzoyl)-3,4-dihydro-1H-isoquinolin-7-yl]propanoic acid;
3-[2-(3,4-dimethylbenzoyl)-3,4-dihydro-1H-isoquinolin-7-yl]-3-(1-ethyl-4-methyl-benzotriazol-5-yl)propanoic acid;
3-[2-(2,3-dimethylbenzoyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-(2,3,5-trimethylbenzoyl)-3,4-dihydro-1H-isoquinolin-7-yl]propanoic acid;
3-[2-(3,5-diethylbenzoyl)-3,4-dihydro-1H-isoquinolin-7-yl]-3-(1-ethyl-4-methyl-benzotriazol-5-yl)propanoic acid;
3-[2-(3,5-dimethylbenzoyl)-3,4-dihydro-1H-isoquinolin-7-yl]-3-(1-ethyl-4-methyl-benzotriazol-5-yl)propanoic acid;
(3S)-3-[2-(3,5-dimethylbenzoyl)-3,4-dihydro-1H-isoquinolin-7-yl]-3-(1-ethyl-4-methyl-benzotriazol-5-yl)propanoic acid;
(3R)-3-[2-(3,5-dimethylbenzoyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-(2,3,5,6-tetramethylbenzoyl)-3,4-dihydro-1H-isoquinolin-7-yl]propanoic acid;
3-[2-(2,6-dimethylbenzoyl)-3,4-dihydro-1H-isoquinolin-7-yl]-3-(1-ethyl-4-methyl-benzotriazol-5-yl)propanoic acid;
3-[2-(2,6-diethylbenzoyl)-3,4-dihydro-1H-isoquinolin-7-yl]-3-(1-ethyl-4-methyl-benzotriazol-5-yl)propanoic acid;
3-[2-(2,6-difluorobenzoyl)-3,4-dihydro-1H-isoquinolin-7-yl]-3-(1-ethyl-4-methyl-benzotriazol-5-yl)propanoic acid;
3-[2-(adamantane-1-carbonyl)-3,4-dihydro-1H-isoquinolin-7-yl]-3-(1-ethyl-4-methyl-benzotriazol-5-yl)propanoic acid;
3-[2-(bicyclo[2.2.2]octane-4-carbonyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-[4-(trifluoromethyl)cyclohexanecarbonyl]-3,4-dihydro-1H-isoquinolin-7-yl]propanoic acid;
3-(1-ethyl-4-methyl-benzotriazol-5-yl)-3-[2-[4-(trifluoromethyl)cyclohexanecarbonyl]-3,4-dihydro-1H-isoquinolin-7-yl]propanoic acid;
3-(1-ethyl-4-methyl-benzotriazol-5-yl)-3-[2-(2-phenylacetyl)-3,4-dihydro-1H-isoquinolin-7-yl]propanoic acid;
(3S)-3-(1-ethyl-4-methyl-benzotriazol-5-yl)-3-[2-(2-phenylacetyl)-3,4-dihydro-1H-isoquinolin-7-yl]propanoic acid;
(3R)-3-(1-ethyl-4-methyl-benzotriazol-5-yl)-3-[2-(2-phenylacetyl)-3,4-dihydro-1H-isoquinolin-7-yl]propanoic acid;
3-(1-ethyl-4-methyl-benzotriazol-5-yl)-3-[2-[(2S)-2-methoxy-2-phenyl-acetyl]-3,4-dihydro-1H-isoquinolin-7-yl]propanoic acid;
3-(1-ethyl-4-methyl-benzotriazol-5-yl)-3-[2-(piperidine-1-carbonyl)-3,4-dihydro-1H-isoquinolin-7-yl]propanoic acid;
3-(1-ethyl-4-methyl-benzotriazol-5-yl)-3-[2-[4-(trifluoromethyl)piperidine-1-carbonyl]-3,4-dihydro-1H-isoquinolin-7-yl]propanoic acid;
3-(1-ethyl-4-methyl-benzotriazol-5-yl)-3-[2-[methyl(phenyl)carbamoyl]-3,4-dihydro-1H-isoquinolin-7-yl]propanoic acid;
3-(1-ethyl-4-methyl-benzotriazol-5-yl)-3-[2-(2-isopropylmorpholine-4-carbonyl)-3,4-dihydro-1H-isoquinolin-7-yl]propanoic acid;
(3S)-3-(1-ethyl-4-methyl-benzotriazol-5-yl)-3-[2-(2,3,5,6-tetramethylbenzoyl)-3,4-dihydro-1H-isoquinolin-7-yl]propanoic acid;
(3R)-3-(1-ethyl-4-methyl-benzotriazol-5-yl)-3-[2-(2,3,5,6-tetramethylbenzoyl)-3,4-dihydro-1H-isoquinolin-7-yl]propanoic acid;
(3S)-3-(1-ethyl-4-methyl-benzotriazol-5-yl)-3-[2-(2-methylbenzoyl)-3,4-dihydro-1H-isoquinolin-7-yl]propanoic acid;
(3R)-3-(1-ethyl-4-methyl-benzotriazol-5-yl)-3-[2-(2-methylbenzoyl)-3,4-dihydro-1H-isoquinolin-7-yl]propanoic acid;
(3S)-3-(2-benzoyl-3,4-dihydro-1H-isoquinolin-7-yl)-3-(1-ethyl-4-methyl-benzotriazol-5-yl)propanoic acid;
(3R)-3-(2-benzoyl-3,4-dihydro-1H-isoquinolin-7-yl)-3-(1-ethyl-4-methyl-benzotriazol-5-yl)propanoic acid;
3-[2-(benzenesulfonyl)-3,4-dihydro-1H-isoquinolin-7-yl]-3-(1-methylbenzotriazol-5-yl)propanoic acid;
3-(2-benzoyl-3,4-dihydro-1H-isoquinolin-7-yl)-3-(1-methylbenzotriazol-5-yl)propanoic acid;
(3S)-3-(2-benzoyl-3,4-dihydro-1H-isoquinolin-7-yl)-3-(1-methylbenzotriazol-5-yl)propanoic acid;
(3R)-3-(2-benzoyl-3,4-dihydro-1H-isoquinolin-7-yl)-3-(1-methylbenzotriazol-5-yl)propanoic acid;
(3S)-3-(1-ethyl-4-methyl-benzotriazol-5-yl)-2-methyl-3-[2-(2,3,5,6-tetramethylbenzoyl)-3,4-dihydro-1H-isoquinolin-7-yl]propanoic acid;
(3S)-3-(1-ethyl-4-methyl-benzotriazol-5-yl)-2,2-dimethyl-3-[2-(2,3,5,6-tetramethylbenzoyl)-3,4-dihydro-1H-isoquinolin-7-yl]propanoic acid;
(2S)-2-[(S)-(1-ethyl-4-methyl-benzotriazol-5-yl)-[2-(2,3,5,6-tetramethylbenzoyl)-3,4-dihydro-1H-isoquinolin-7-yl]methyl]butanoic acid;
(2R)-2-[(S)-(1-ethyl-4-methyl-benzotriazol-5-yl)-[2-(2,3,5,6-tetramethylbenzoyl)-3,4-dihydro-1H-isoquinolin-7-yl]methyl]butanoic acid;
3-(6-methoxy-4-methyl-3-pyridyl)-3-[2-(2,3,5,6-tetramethylbenzoyl)-3,4-dihydro-1H-isoquinolin-7-yl]propanoic acid;
(3S)-3-(6-methoxy-4-methyl-3-pyridyl)-3-[2-(2,3,5,6-tetramethylbenzoyl)-3,4-dihydro-1H-isoquinolin-7-yl]propanoic acid;
(3R)-3-(6-methoxy-4-methyl-3-pyridyl)-3-[2-(2,3,5,6-tetramethylbenzoyl)-3,4-dihydro-1H-isoquinolin-7-yl]propanoic acid;
3-(4-cyano-2-methyl-phenyl)-3-[2-(2,3,5,6-tetramethylbenzoyl)-3,4-dihydro-1H-isoquinolin-7-yl]propanoic acid;
3-(4-carbamoyl-2-methyl-phenyl)-3-[2-(2,3,5,6-tetramethylbenzoyl)-3,4-dihydro-1H-isoquinolin-7-yl]propanoic acid;
3-(2-benzoyl-3,4-dihydro-1H-isoquinolin-7-yl)-3-(4-cyano-2-methyl-phenyl)propanoic acid;
3-[(1S)-2-benzoyl-1-methyl-3,4-dihydro-1H-isoquinolin-7-yl]-3-(1-ethyl-4-methyl-benzotriazol-5-yl)propanoic acid;
3-(2-benzoyl-1,3,4,5-tetrahydro-2-benzazepin-8-yl)-3-(1-ethyl-4-methyl-benzotriazol-5-yl)propanoic acid;
(3S)-3-[(4S)-2-benzoyl-4-methyl-3,4-dihydro-1H-isoquinolin-7-yl]-3-(1-ethyl-4-methyl-benzotriazol-5-yl)propanoic acid; N-ethylethanamine
(3R)-3-[(4S)-2-benzoyl-4-methyl-3,4-dihydro-1H-isoquinolin-7-yl]-3-(1-ethyl-4-methyl-benzotriazol-5-yl)propanoic acid;
(3S)-3-[(4R)-2-benzoyl-4-methyl-3,4-dihydro-1H-isoquinolin-7-yl]-3-(1-ethyl-4-methyl-benzotriazol-5-yl)propanoic acid;
(3R)-3-[(4R)-2-benzoyl-4-methyl-3,4-dihydro-1H-isoquinolin-7-yl]-3-(1-ethyl-4-methyl-benzotriazol-5-yl)propanoic acid;
(3S)-3-(2-benzoyl-5-methyl-3,4-dihydro-1H-isoquinolin-7-yl)-3-(1-ethyl-4-methyl-benzotriazol-5-yl)propanoic acid; N-ethylethanamine
(3R)-3-(2-benzoyl-5-methyl-3,4-dihydro-1H-isoquinolin-7-yl)-3-(1-ethyl-4-methyl-benzotriazol-5-yl)propanoic acid; N-ethylethanamine
3-(2-benzoyl-5-nitro-3,4-dihydro-1H-isoquinolin-7-yl)-3-(1-ethyl-4-methyl-benzotriazol-5-yl)propanoic acid;
(3S)-3-(1-ethyl-4-methyl-benzotriazol-5-yl)-3-[5-methyl-2-(2,3,5,6-tetramethylbenzoyl)-3,4-dihydro-1H-isoquinolin-7-yl]propanoic acid;
(3R)-3-(1-ethyl-4-methyl-benzotriazol-5-yl)-3-[5-methyl-2-(2,3,5,6-tetramethylbenzoyl)-3,4-dihydro-1H-isoquinolin-7-yl]propanoic acid;
(3S)-3-(1-ethyl-4-methyl-benzotriazol-5-yl)-3-[2-(naphthalene-2-carbonyl)-3,4-dihydro-1H-isoquinolin-7-yl]propanoic acid;
(3R)-3-(1-ethyl-4-methyl-benzotriazol-5-yl)-3-[2-(naphthalene-2-carbonyl)-3,4-dihydro-1H-isoquinolin-7-yl]propanoic acid;
3-(2-benzoyl-3-methyl-3,4-dihydro-1H-isoquinolin-7-yl)-3-(1-ethyl-4-methyl-benzotriazol-5-yl)propanoic acid;
3-(2-benzoyl-3-methyl-1,2,3,4-tetrahydroisoquinolin-7-yl)-3-(1-ethyl-4-methyl-1H-benzo[d][1,2,3]triazol-5-yl)propanoic acid;
3-(1-ethyl-4-methyl-benzotriazol-5-yl)-3-[2-(isoquinoline-3-carbonyl)-3,4-dihydro-1H-isoquinolin-7-yl]propanoic acid;
3-(1-ethyl-4-methyl-benzotriazol-5-yl)-3-[2-(2-methylthiazole-4-carbonyl)-3,4-dihydro-1H-isoquinolin-7-yl]propanoic acid;
3-[2-(1H-benzimidazole-2-carbonyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-(6-methylpyridine-2-carbonyl)-3,4-dihydro-1H-isoquinolin-7-yl]propanoic acid;
3-(1-ethyl-4-methyl-benzotriazol-5-yl)-3-[2-(2-methylpyridine-3-carbonyl)-3,4-dihydro-1H-isoquinolin-7-yl]propanoic acid;
3-[2-(2,5-dimethylbenzoyl)-3,4-dihydro-1H-isoquinolin-7-yl]-3-(1-ethyl-4-methyl-benzotriazol-5-yl)propanoic acid;
3-(1-ethyl-4-methyl-benzotriazol-5-yl)-3-[2-[2-(trifluoromethyl)benzoyl]-3,4-dihydro-1H-isoquinolin-7-yl]propanoic acid;
3-(1-ethyl-4-methyl-benzotriazol-5-yl)-3-[2-(2-fluoro-5-methyl-benzoyl)-3,4-dihydro-1H-isoquinolin-7-yl]propanoic acid;
3-(1-ethyl-4-methyl-benzotriazol-5-yl)-3-[2-(1H-indole-5-carbonyl)-3,4-dihydro-1H-isoquinolin-7-yl]propanoic acid; and
3-[2-(3-cyanobenzoyl)-3,4-dihydro-1H-isoquinolin-7-yl]-3-(1-ethyl-4-methyl-benzotriazol-5-yl)propanoic acid,
and a pharmaceutically acceptable salt thereof.

40. A pharmaceutical composition comprising at least one compound of any one of claims 1-39, or a pharmaceutically acceptable salt thereof, and at least one pharmaceutically acceptable excipient.

41. A method of activating Nrf2 in a subject in need thereof, comprising administering to the subject a therapeutically effective amount of at least one compound according to any one of claims 1-39, or a pharmaceutically acceptable salt thereof.

42. A method of treating a disease caused by oxidative stress in a subject, the method comprising administering to the subject a therapeutically effective amount of a compound according to any one of claims 1-39, or a pharmaceutically acceptable salt thereof.

43. A method of treating a disorder in a subject, wherein the disorder is selected from the group consisting of a neurodegenerative disease, inflammation/an inflammatory disease, an autoimmune disease, an ischemic fibrotic disease, a cancer, premature aging, a cardiovascular disease, a liver disease, a hemoglobinopathy, thalassemia (e.g., beta-thalassemia), and a metabolic disorder, the method comprising administering to the subject a therapeutically effective amount of a compound according to any one of claims 1-39, or a pharmaceutically acceptable salt thereof.

44. A method for treating a neurodegenerative disorder in a subject, wherein the disorder is selected from the group consisting of Alzheimer's disease, Parkinson's disease, Huntington disease, amyotrophic lateral sclerosis, diffuse Lewy body disease, chorea-acanthocytosis, primary lateral sclerosis, multiple sclerosis, frontotemporal dementia, Friedreich's ataxia, and epilepsy, the method comprising administering to the subject a therapeutically effective amount of a compound according to any one of claims 1-39, or a pharmaceutically acceptable salt thereof.

45. The method of claim 44, wherein the disorder is Parkinson's disease or amyotrophic lateral sclerosis.

46. A method for treating sickle cell disease or thalassemia (e.g., beta-thalassemia) in a subject, the method comprising administering to the subject a therapeutically effective amount of a compound according to any one of claims 1-39, or a pharmaceutically acceptable salt thereof.

47. A method for treating sickle cell disease in a subject, the method comprising administering to the subject a therapeutically effective amount of a compound according to any one of claims 1-39, or a pharmaceutically acceptable salt thereof.
